(19) United States
(12) Patent Application Publication
(10) Pub. No.: US 2026/0190856 A1
(43) Pub. Date: Jul. 2, 2026

(54) ORGANIC ELECTROLUMINESCENT COMPOUND AND ORGANIC ELECTROLUMINESCENT DEVICE COMPRISING THE SAME

(71) Applicant: DuPont Specialty Materials Korea Ltd., Chungcheongnam-do (KR)

(72) Inventors: Eun-Joung CHOI, Gyeonggi-do (KR); So-Mi PARK, Gyeonggi-do (KR); Seung-Hyun YOON, Gyeonggi-do (KR); HaeYeon KIM, Gyeonggi-do (KR)

(21) Appl. No.: 19/417,658

(22) Filed: Dec. 12, 2025

(30) Foreign Application Priority Data
Dec. 27, 2024 (KR) 10-2024-0198785
Aug. 18, 2025 (KR) 10-2025-0114035
Dec. 1, 2025 (KR) 10-2025-0187009

(51) Int. Cl. H10K 85/60; C07D 213/38; C07D 401/14; C07D 471/04; C09K 11/06; H10K 50/11

(52) U.S. Cl. CPC H10K 85/654; C07D 213/38; C07D 401/14; C07D 471/04; C09K 11/06; H10K 85/615; H10K 85/6572; C09K 2211/1007; C09K 2211/1011; C09K 2211/1018; H10K 50/11

(57) ABSTRACT

The present disclosure relates to an organic electroluminescent compound represented by Formula 1, and an organic electroluminescent device comprising the same. It is possible to provide an organic electroluminescent device having low driving voltage and/or high current efficiency and/or improved lifespan characteristics compared with conventional organic electroluminescent devices.

### TECHNICAL FIELD

The present disclosure relates to an organic electroluminescent compound and an organic electroluminescent device comprising the same.

### BACKGROUND ART

The TPD/Alq3 bilayer small molecule organic electroluminescent device (OLED) with green emission, which is constituted with a light-emitting layer and a charge transport layer, was first developed by Tang et al. of Eastman Kodak in 1987.

Thereafter, studies on OLEDs have been rapidly progressed, and OLEDs have been commercialized. At present, an organic electroluminescent device mainly uses phosphorescent materials having excellent luminous efficiency in panel realization.

Accordingly, for prolonged use and high resolution of display, an OLED having high luminous efficiency is necessary.

Korean Patent Application Laid-Open No. 2023-0151982 discloses compounds comprising a phenanthroline derivative. However, this reference does not specifically disclose specific compounds to which the present disclosure is directed. There is a continuing need to develop organic electroluminescent compounds having improved performance, such as improved driving voltage and/or luminous efficiency and/or lifespan characteristics, compared to previously disclosed compounds.

### DISCLOSURE OF INVENTION

### Technical Problem

The object of the present disclosure is to provide an organic electroluminescent compound with a novel structure suitable for application to an organic electroluminescent device. Another object of the present disclosure is to provide an organic electroluminescent device with low driving voltage and/or high current efficiency and/or improved lifespan characteristics.

### Solution to Problem

As a result of intensive studies to solve the technical problems above, the present inventors found that the aforementioned object can be achieved by an organic electroluminescent compound represented by the following Formula 1 and an organic electroluminescent device comprising the same, thereby completing the present invention.

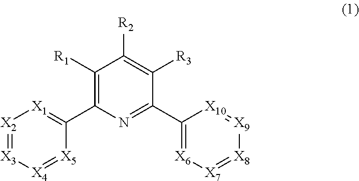

In Formula 1,
X₁ to X₁₀ each independently represent CR₄ or N;
R₁ to R₄ each independently represent hydrogen, deuterium, a halogen, a cyano, a substituted or unsubstituted (C₁-C₃₀)alkyl, a substituted or unsubstituted (C₂-C₃₀)alkenyl, a substituted or unsubstituted (C₃-C₃₀)cycloalkyl, a substituted or unsubstituted (C₃-C₃₀)cycloalkenyl, a substituted or unsubstituted (3- to 7-membered)heterocycloalkyl, or -(L)ₐ-HAr;
at least one of R₁ to R₄ is -(L)ₐ-HAr;
when R₂ is -(L)ₐ-HAr, each R₄ independently represents hydrogen, deuterium, or a cyano group;
each L independently represents a single bond, a substituted or unsubstituted (C₆-C₃₀)arylene, a substituted or unsubstituted (C₂-C₃₀)alkenylene, a substituted or unsubstituted (C₂-C₃₀)alkynylene, or a substituted or unsubstituted (3- to 30-membered)heteroarylene;
a is an integer of 1 or 2, and when a is 2, L may be the same as or different from the other; and
HAr represents a substituted or unsubstituted (3- to 30-membered)heteroaryl comprising at least one nitrogen atom;
provided that where R₂ is -(L)ₐ-HAr and HAr is a substituted or unsubstituted phenanthrolinyl, L is linked to any one of positions 3 to 8 of the phenanthrolinyl when L is not a substituted or unsubstituted (C₂-C₃₀)alkenylene or a substituted or unsubstituted (C₂-C₃₀)alkynylene.

### Advantageous Effects of Invention

The organic electroluminescent compound according to the present disclosure exhibits performance suitable for use in an organic electroluminescent device. By using the organic electroluminescent compound according to the present disclosure in the N-type charge generation layer, an organic electroluminescent device with low driving voltage and/or high current efficiency and/or improved lifespan characteristics as compared with conventional organic electroluminescent devices can be provided. Additionally, it is possible to produce a display device or lighting device using the same.

### MODE FOR INVENTION

Hereinafter, the present disclosure will be described in detail. However, the following description is intended to explain the present disclosure, and is not meant in any way to restrict the scope of the present disclosure.

The “organic electroluminescent compound” in the present disclosure refers to a compound that may be used in an organic electroluminescent device, and may be included in any layer constituting an organic electroluminescent device, as necessary.

Herein, the “(C₁-C₃₀)alkyl” is meant to be a linear or branched alkyl having 1 to 30 carbon atoms constituting the chain, in which the number of carbon atoms is preferably 1 to 20, and more preferably 1 to 10. Specific examples of the alkyl include methyl, ethyl, n-propyl, isopropyl, n-butyl, isobutyl, tert-butyl, sec-butyl, etc. Herein, the “(C₃-C₃₀)cycloalkyl” is meant to be a mono- or polycyclic hydrocarbon having 3 to 30 ring skeleton carbon atoms, in which the number of carbon atoms is preferably 3 to 20, and more preferably 3 to 7. Examples of the cycloalkyl include cyclopropyl, cyclobutyl, cyclopentyl, cyclohexyl, cyclopentylmethyl, cyclohexylmethyl, norbornyl, adamantyl, etc. Herein, the “(3- to 7-membered)heterocycloalkyl” is meant to be a saturated or partially unsaturated mono- or polycyclic ring hydrocarbon substituent having 3 to 7 ring skeleton atoms, preferably having 5 to 7 carbon atoms, and including at least one heteroatom selected from the group consisting of B, N, O, S, Si, P, and Se. The heterocycloalkyl includes, for example, tetrahydrofuran, pyrrolidine, thiolane, tetrahydropyran, etc.

Herein, the “(C₂-C₃₀)alkenyl(ene)” or “(C₂-C₃₀)alkynyl(ene)” is meant to be a linear or branched alkenyl group or alkenylene group, or alkynyl group or alkynylene group having 2 to 30 carbon atoms constituting the chain, in which the number of carbon atoms is preferably 2 to 20, and more preferably 2 to 10. The alkenyl group refers to a substituent including at least one double bond, and the alkynyl group refers to a substituent including at least one triple bond, which may be present at the terminal or internal position. Examples of the alkenyl include ethenyl, propenyl, butenyl, pentenyl, etc., and examples of the alkynyl include ethynyl, propynyl, butynyl, pentynyl, etc., which may have a linear or branched structure.

Herein, the “(C₆-C₃₀)aryl” or “(C₆-C₃₀)arylene” is meant to be a monocyclic or fused ring-type radical derived from an aromatic hydrocarbon having 6 to 30, preferably 6 to 20, and more preferably 6 to 15 ring skeleton carbon atoms, which may be partially saturated. The aryl may include a spiro structure. Examples of the aryl may include phenyl, biphenyl, terphenyl, quaterphenyl, naphthyl, binaphthyl, phenylnaphthyl, naphthylphenyl, fluorenyl, phenylfluorenyl, dimethylfluorenyl, diphenylfluorenyl, benzofluorenyl, diphenylbenzofluorenyl, dibenzofluorenyl, phenanthrenyl, benzophenanthrenyl, phenylphenanthrenyl, anthracenyl, benzanthracenyl, indenyl, triphenylenyl, pyrenyl, tetracenyl, perylenyl, chrysenyl, benzochrysenyl, naphthacenyl, fluoranthenyl, benzofluoranthenyl, tolyl, xylyl, mesityl, cumenyl, spiro[fluorene-fluoren]yl, spiro[fluorene-benzofluoren]yl, azulenyl, tetramethyl-dihydrophenanthrenyl, etc. More specifically, examples of the aryl may include o-tolyl, m-tolyl, p-tolyl, 2,3-xylyl, 3,4-xylyl, 2,5-xylyl, mesityl, o-cumenyl, m-cumenyl, p-cumenyl, p-t-butylphenyl, p-(2-phenylpropyl)phenyl, 4′-methylbiphenyl, 4″-t-butyl-p-terphenyl-4-yl, o-biphenyl, m-biphenyl, p-biphenyl, o-terphenyl, m-terphenyl-4-yl, m-terphenyl-3-yl, m-terphenyl-2-yl, p-terphenyl-4-yl, p-terphenyl-3-yl, p-terphenyl-2-yl, m-quaterphenyl, 1-naphthyl, 2-naphthyl, 1-fluorenyl, 2-fluorenyl, 3-fluorenyl, 4-fluorenyl, 9-fluorenyl, 9,9-dimethyl-1-fluorenyl, 9,9-dimethyl-2-fluorenyl, 9,9-dimethyl-3-fluorenyl, 9,9-dimethyl-4-fluorenyl, 9,9-diphenyl-1-fluorenyl, 9,9-diphenyl-2-fluorenyl, 9,9-diphenyl-3-fluorenyl, 9,9-diphenyl-4-fluorenyl, 1-anthryl, 2-anthryl, 9-anthryl, 1-phenanthryl, 2-phenanthryl, 3-phenanthryl, 4-phenanthryl, 9-phenanthryl, 1-chrysenyl, 2-chrysenyl, 3-chrysenyl, 4-chrysenyl, 5-chrysenyl, 6-chrysenyl, benzo[c]phenanthryl, benzo[g]chrysenyl, 1-triphenylenyl, 2-triphenylenyl, 3-triphenylenyl, 4-triphenylenyl, 3-fluoranthenyl, 4-fluoranthenyl, 8-fluoranthenyl, 9-fluoranthenyl, benzofluoranthenyl, 11,11-dimethyl-1-benzo[a]fluorenyl, 11,11-dimethyl-2-benzo[a]fluorenyl, 11,11-dimethyl-3-benzo[a]fluorenyl, 11,11-dimethyl-4-benzo[a]fluorenyl, 11,11-dimethyl-5-benzo[a]fluorenyl, 11,11-dimethyl-6-benzo[a]fluorenyl, 11,11-dimethyl-7-benzo[a]fluorenyl, 11,11-dimethyl-8-benzo[a]fluorenyl, 11,11-dimethyl-9-benzo[a]fluorenyl, 11,11-dimethyl-10-benzo[a]fluorenyl, 11,11-dimethyl-1-benzo[b]fluorenyl, 11,11-dimethyl-2-benzo[b]fluorenyl, 11,11-dimethyl-3-benzo[b]fluorenyl, 11,11-dimethyl-4-benzo[b]fluorenyl, 11,11-dimethyl-5-benzo[b]fluorenyl, 11,11-dimethyl-6-benzo[b]fluorenyl, 11,11-dimethyl-7-benzo[b]fluorenyl, 11,11-dimethyl-8-benzo[b]fluorenyl, 11,11-dimethyl-9-benzo[b]fluorenyl, 11,11-dimethyl-10-benzo[b]fluorenyl, 11,11-dimethyl-1-benzo[c]fluorenyl, 11,11-dimethyl-2-benzo[c]fluorenyl, 11,11-dimethyl-3-benzo[c]fluorenyl, 11,11-dimethyl-4-benzo[c]fluorenyl, 11,11-dimethyl-5-benzo[c]fluorenyl, 11,11-dimethyl-6-benzo[c]fluorenyl, 11,11-dimethyl-7-benzo[c]fluorenyl, 11,11-dimethyl-8-benzo[c]fluorenyl, 11,11-dimethyl-9-benzo[c]fluorenyl, 11,11-dimethyl-10-benzo[c]fluorenyl, 11,11-diphenyl-1-benzo[a]fluorenyl, 11,11-diphenyl-2-benzo[a]fluorenyl, 11,11-diphenyl-3-benzo[a]fluorenyl, 11,11-diphenyl-4-benzo[a]fluorenyl, 11,11-diphenyl-5-benzo[a]fluorenyl, 11,11-diphenyl-6-benzo[a]fluorenyl, 11,11-diphenyl-7-benzo[a]fluorenyl, 11,11-diphenyl-8-benzo[a]fluorenyl, 11,11-diphenyl-9-benzo[a]fluorenyl, 11,11-diphenyl-10-benzo[a]fluorenyl, 11,11-diphenyl-1-benzo[b]fluorenyl, 11,11-diphenyl-2-benzo[b]fluorenyl, 11,11-diphenyl-3-benzo[b]fluorenyl, 11,11-diphenyl-4-benzo[b]fluorenyl, 11,11-diphenyl-5-benzo[b]fluorenyl, 11,11-diphenyl-6-benzo[b]fluorenyl, 11,11-diphenyl-7-benzo[b]fluorenyl, 11,11-diphenyl-8-benzo[b]fluorenyl, 11,11-diphenyl-9-benzo[b]fluorenyl, 11,11-diphenyl-10-benzo[b]fluorenyl, 11,11-diphenyl-1-benzo[c]fluorenyl, 11,11-diphenyl-2-benzo[c]fluorenyl, 11,11-diphenyl-3-benzo[c]fluorenyl, 11,11-diphenyl-4-benzo[c]fluorenyl, 11,11-diphenyl-5-benzo[c]fluorenyl, 11,11-diphenyl-6-benzo[c]fluorenyl, 11,11-diphenyl-7-benzo[c]fluorenyl, 11,11-diphenyl-8-benzo[c]fluorenyl, 11,11-diphenyl-9-benzo[c]fluorenyl, 11,11-diphenyl-10-benzo[c]fluorenyl, 9,9,10,10-tetramethyl-9,10-dihydro-1-phenanthrenyl, 9,9,10,10-tetramethyl-9,10-dihydro-2-phenanthrenyl, 9,9,10,10-tetramethyl-9,10-dihydro-3-phenanthrenyl, 9,9,10,10-tetramethyl-9,10-dihydro-4-phenanthrenyl, etc.

Herein, the “(3- to 30-membered)heteroaryl” or “(3- to 30-membered)heteroarylene” is meant to be an aryl group or arylene group having 3 to 30 ring skeleton atoms, and including at least one heteroatom selected from the group consisting of B, N, O, S, Si, P, and Se, in which the number of ring skeleton atoms is preferably 3 to 30, and more preferably 5 to 20. Herein, the number of the heteroatoms is preferably 1 to 4. The heteroaryl or heteroarylene may be a monocyclic ring-type or a fused ring-type condensed with at least one benzene ring, and may be partially saturated. In addition, the heteroaryl or heteroarylene may be one formed by linking at least one heteroaryl or aryl group to a heteroaryl group via a single bond(s), and may comprise a spiro structure. Examples of the heteroaryl may be a monocyclic ring-type heteroaryl such as furyl, thiophenyl, pyrrolyl, imidazolyl, pyrazolyl, thiazolyl, thiadiazolyl, isothiazolyl, isoxazolyl, oxazolyl, oxadiazolyl, triazinyl, tetrazinyl, triazolyl, tetrazolyl, furazanyl, pyridyl, pyrazinyl, pyrimidinyl, pyridazinyl, etc., and a fused ring-type heteroaryl such as benzofuranyl, benzothiophenyl, isobenzofuranyl, dibenzofuranyl, dibenzothiophenyl, benzofuroquinolinyl, benzofuroquinazolinyl, benzofuronaphthyridinyl, benzofuropyrimidinyl, naphthofuropyrimidinyl, benzothienoquinolinyl, benzothienoquinazolinyl, benzothienonaphthyridinyl, benzothienopyrimidinyl, naphthothienopyrimidinyl, pyrimidoindolyl, benzopyrimidoindolyl, benzofuropyrazinyl, naphthofuropyrazinyl, benzothienopyrazinyl, naphthothienopyrazinyl, pyrazinoindolyl, benzopyrazinoindolyl, benzoimidazolyl, benzothiazolyl, benzoisothiazolyl, benzoisoxazolyl, benzooxazolyl, imidazopyridinyl, isoindolyl, indolyl, benzoindolyl, indazolyl, benzothiadiazolyl, quinolyl, isoquinolyl, cinnolinyl, quinazolinyl, quinoxalinyl, carbazolyl, azacarbazolyl, benzocarbazolyl, dibenzocarbazolyl, phenoxazinyl, phenanthridinyl, benzodioxolyl, indolizidinyl, acridinyl, silafluorenyl, germafluorenyl, benzotriazolyl, phenazinyl, imidazopyridinyl, chromenoquinazolinyl, thiochromenoquinazolinyl, dimethylbenzopyrimidinyl, indolocarbazolyl, indenocarbazolyl, etc. More specifically, the heteroaryl may include 1-pyrrolyl, 2-pyrrolyl, 3-pyrrolyl, 2-pyridinyl, 3-pyridinyl, 4-pyridinyl, 2-pyrimidinyl, 4-pyrimidinyl, 5-pyrimidinyl, 6-pyrimidinyl, 1,2,3-triazin-4-yl, 1,2,4-triazin-3-yl, 1,3,5-triazin-2-yl, 1-imidazolyl, 2-imidazolyl, 1-pyrazolyl, 1-indolizidinyl, 2-indolizidinyl, 3-indolizidinyl, 5-indolizidinyl, 6-indolizidinyl, 7-indolizidinyl, 8-indolizidinyl, 2-imidazopyridinyl, 3-imidazopyridinyl, 5-imidazopyridinyl, 6-imidazopyridinyl, 7-imidazopyridinyl, 8-imidazopyridinyl, 1-indolyl, 2-indolyl, 3-indolyl, 4-indolyl, 5-indolyl, 6-indolyl, 7-indolyl, 1-isoindolyl, 2-isoindolyl, 3-isoindolyl, 4-isoindolyl, 5-isoindolyl, 6-isoindolyl, 7-isoindolyl, 2-furyl, 3-furyl, 2-benzofuranyl, 3-benzofuranyl, 4-benzofuranyl, 5-benzofuranyl, 6-benzofuranyl, 7-benzofuranyl, 1-isobenzofuranyl, 3-isobenzofuranyl, 4-isobenzofuranyl, 5-isobenzofuranyl, 6-isobenzofuranyl, 7-isobenzofuranyl, 2-quinolyl, 3-quinolyl, 4-quinolyl, 5-quinolyl, 6-quinolyl, 7-quinolyl, 8-quinolyl, 1-isoquinolyl, 3-isoquinolyl, 4-isoquinolyl, 5-isoquinolyl, 6-isoquinolyl, 7-isoquinolyl, 8-isoquinolyl, 2-quinoxalinyl, 5-quinoxalinyl, 6-quinoxalinyl, 1-carbazolyl, 2-carbazolyl, 3-carbazolyl, 4-carbazolyl, 9-carbazolyl, azacarbazol-1-yl, azacarbazol-2-yl, azacarbazol-3-yl, azacarbazol-4-yl, azacarbazol-5-yl, azacarbazol-6-yl, azacarbazol-7-yl, azacarbazol-8-yl, azacarbazol-9-yl, 1-phenanthridinyl, 2-phenanthridinyl, 3-phenanthridinyl, 4-phenanthridinyl, 6-phenanthridinyl, 7-phenanthridinyl, 8-phenanthridinyl, 9-phenanthridinyl, 10-phenanthridinyl, 1-acridinyl, 2-acridinyl, 3-acridinyl, 4-acridinyl, 9-acridinyl, 2-oxazolyl, 4-oxazolyl, 5-oxazolyl, 2-oxadiazolyl, 5-oxadiazolyl, 3-furazanyl, 2-thienyl, 3-thienyl, 2-methylpyrrol-1-yl, 2-methylpyrrol-3-yl, 2-methylpyrrol-4-yl, 2-methylpyrrol-5-yl, 3-methylpyrrol-1-yl, 3-methylpyrrol-2-yl, 3-methylpyrrol-4-yl, 3-methylpyrrol-5-yl, 2-t-butylpyrrol-4-yl, 3-(2-phenylpropyl)pyrrol-1-yl, 2-methyl-1-indolyl, 4-methyl-1-indolyl, 2-methyl-3-indolyl, 4-methyl-3-indolyl, 2-t-butyl-1-indolyl, 4-t-butyl-1-indolyl, 2-t-butyl-3-indolyl, 4-t-butyl-3-indolyl, 1-dibenzofuranyl, 2-dibenzofuranyl, 3-dibenzofuranyl, 4-dibenzofuranyl, 1-dibenzothiophenyl, 2-dibenzothiophenyl, 3-dibenzothiophenyl, 4-dibenzothiophenyl, 1-naphtho[1,2-b]benzofuranyl, 2-naphtho[1,2-b]benzofuranyl, 3-naphtho[1,2-b]benzofuranyl, 4-naphtho[1,2-b]benzofuranyl, 5-naphtho[1,2-b]benzofuranyl, 6-naphtho[1,2-b]benzofuranyl, 7-naphtho[1,2-b]benzofuranyl, 8-naphtho[1,2-b]benzofuranyl, 9-naphtho[1,2-b]benzofuranyl, 10-naphtho[1,2-b]benzofuranyl, 1-naphtho[2,3-b]benzofuranyl, 2-naphtho[2,3-b]benzofuranyl, 3-naphtho[2,3-b]benzofuranyl, 4-naphtho[2,3-b]benzofuranyl, 5-naphtho[2,3-b]benzofuranyl, 6-naphtho[2,3-b]benzofuranyl, 7-naphtho[2,3-b]benzofuranyl, 8-naphtho[2,3-b]benzofuranyl, 9-naphtho[2,3-b]benzofuranyl, 10-naphtho[2,3-b]benzofuranyl, 1-naphtho[2,1-b]benzofuranyl, 2-naphtho[2,1-b]benzofuranyl, 3-naphtho[2,1-b]benzofuranyl, 4-naphtho[2,1-b]benzofuranyl, 5-naphtho[2,1-b]benzofuranyl, 6-naphtho[2,1-b]benzofuranyl, 7-naphtho[2,1-b]benzofuranyl, 8-naphtho[2,1-b]benzofuranyl, 9-naphtho[2,1-b]benzofuranyl, 10-naphtho[2,1-b]benzofuranyl, 1-naphtho[1,2-b]benzothiophenyl, 2-naphtho[1,2-b]benzothiophenyl, 3-naphtho[1,2-b]benzothiophenyl, 4-naphtho[1,2-b]benzothiophenyl, 5-naphtho[1,2-b]benzothiophenyl, 6-naphtho[1,2-b]benzothiophenyl, 7-naphtho[1,2-b]benzothiophenyl, 8-naphtho[1,2-b]benzothiophenyl, 9-naphtho[1,2-b]benzothiophenyl, 10-naphtho[1,2-b]benzothiophenyl, 1-naphtho[2,3-b]benzothiophenyl, 2-naphtho[2,3-b]benzothiophenyl, 3-naphtho[2,3-b]benzothiophenyl, 4-naphtho[2,3-b]benzothiophenyl, 5-naphtho[2,3-b]benzothiophenyl, 1-naphtho[2,1-b]benzothiophenyl, 2-naphtho[2,1-b]benzothiophenyl, 3-naphtho[2,1-b]benzothiophenyl, 4-naphtho[2,1-b]benzothiophenyl, 5-naphtho[2,1-b]benzothiophenyl, 6-naphtho[2,1-b]benzothiophenyl, 7-naphtho[2,1-b]benzothiophenyl, 8-naphtho[2,1-b]benzothiophenyl, 9-naphtho[2,1-b]benzothiophenyl, 10-naphtho[2,1-b]benzothiophenyl, 2-benzofuro[3,2-d]pyrimidinyl, 6-benzofuro[3,2-d]pyrimidinyl, 7-benzofuro[3,2-d]pyrimidinyl, 8-benzofuro[3,2-d]pyrimidinyl, 9-benzofuro[3,2-d]pyrimidinyl, 2-benzothio[3,2-d]pyrimidinyl, 6-benzothio[3,2-d]pyrimidinyl, 7-benzothio[3,2-d]pyrimidinyl, 8-benzothio[3,2-d]pyrimidinyl, 9-benzothio[3,2-d]pyrimidinyl, 2-benzofuro[3,2-d]pyrazinyl, 6-benzofuro[3,2-d]pyrazinyl, 7-benzofuro[3,2-d]pyrazinyl, 8-benzofuro[3,2-d]pyrazinyl, 9-benzofuro[3,2-d]pyrazinyl, 2-benzothio[3,2-d]pyrazinyl, 6-benzothio[3,2-d]pyrazinyl, 7-benzothio[3,2-d]pyrazinyl, 8-benzothio[3,2-d]pyrazinyl, 9-benzothio[3,2-d]pyrazinyl, 1-silafluorenyl, 2-silafluorenyl, 3-silafluorenyl, 4-silafluorenyl, 1-germafluorenyl, 2-germafluorenyl, 3-germafluorenyl, 4-germafluorenyl, 1-dibenzoselenophenyl, 2-dibenzoselenophenyl, 3-dibenzoselenophenyl, 4-dibenzoselenophenyl, etc. In addition, “heteroaryl(ene)” can be classified into a heteroaryl(ene) with electronic properties and a heteroaryl(ene) with hole properties. The heteroaryl(ene) with electronic properties is a substituent that is relatively rich in electrons in the parent nucleus, which may be, for example, a substituted or unsubstituted pyridinyl, a substituted or unsubstituted pyrimidinyl, a substituted or unsubstituted triazinyl, a substituted or unsubstituted quinazolinyl, a substituted or unsubstituted quinoxalinyl, a substituted or unsubstituted quinolyl, etc. The heteroaryl(ene) with hole properties is a substituent that is relatively electron-deficient in the parent nucleus, which may be, for example, a substituted or unsubstituted carbazolyl, a substituted or unsubstituted dibenzofuranyl, a substituted or unsubstituted dibenzothiophenyl, etc.

Herein, “a fused ring group of a (C₃-C₃₀)aliphatic ring(s) and a (C₆-C₃₀)aromatic ring(s)” is meant to be a functional group of a ring formed by fusing at least one aliphatic ring having 3 to 30, preferably 3 to 25, and more preferably 3 to 18 ring skeleton carbon atoms, and at least one aromatic ring having 6 to 30, preferably 6 to 25, and more preferably 6 to 18 ring skeleton carbon atoms. For example, the fused ring group may include a fused ring group of at least one benzene and at least one cyclohexane, or a fused ring group of at least one naphthalene and at least one cyclopentane, etc. Herein, the carbon atoms in the fused ring group of a (C₃-C₃₀)aliphatic ring(s) and a (C₆-C₃₀)aromatic ring(s) may be replaced with at least one heteroatom selected from B, N, O, S, Si, P and Se. The “halogen” in the present disclosure includes F, Cl, Br, and I.

In addition, “ortho-” (“o-”), “meta-” (“m-”), and “para-” (“p-”) are prefixes which each represent the relative positions of substituents. The prefix “ortho-” indicates that two substituents are adjacent to each other, and for example, when two substituents in a benzene derivative occupy positions 1 and 2, this is called an “ortho-” configuration. The prefix “meta-” indicates that two substituents are at positions 1 and 3, and for example, when two substituents in a benzene derivative occupy positions 1 and 3, this is called a “meta-” configuration. The prefix “para-” indicates that two substituents are at positions 1 and 4, and for example, when two substituents in a benzene derivative occupy positions 1 and 4, this is called a “para-” configuration.

Herein, “substituted” in the expression “substituted or unsubstituted” means that a hydrogen atom in a certain functional group is replaced with another atom or another functional group, i.e., a substituent. The substituent also includes those in which two or more substituents are linked. Unless otherwise specified, the substituent may replace hydrogen at a position where the substituent can be substituted without limitation, and when two or more hydrogen atoms in a certain functional group are each replaced with a substituent, each substituent may be the same as or different from one another. The maximum number of substituents that can be substituted for a certain functional group may be the total number of valences that can be substituted for each atom forming the functional group. In the formulas of the present disclosure, the substituted alkyl, the substituted alkenyl(ene), the substituted alkynyl(ene), the substituted aryl(ene), the substituted heteroaryl(ene), the substituted cycloalkyl, the substituted cycloalkenyl, and the substituted heterocycloalkyl may each independently be substituted with at least one selected from the group consisting of deuterium, a halogen, a cyano, carboxyl, nitro, hydroxyl, (C₁-C₃₀)alkyl, halo(C₁-C₃₀)alkyl, (C₂-C₃₀)alkenyl, (C₂-C₃₀)alkynyl, (C₁-C₃₀)alkoxy, (C₁-C₃₀)alkylthio, (C₃-C₃₀)cycloalkyl, (C₃-C₃₀)cycloalkenyl, (3- to 7-membered)heterocycloalkyl, (C₆-C₃₀)aryloxy, (C₆-C₃₀)arylthio, (3- to 30-membered)heteroaryl, (C₆-C₃₀)aryl, tri(C₁-C₃₀)alkylsilyl, tri(C₆-C₃₀)arylsilyl, di(C₁-C₃₀)alkyl(C₆-C₃₀)arylsilyl, (C₁-C₃₀)alkyldi(C₆-C₃₀)arylsilyl, a fused ring group of a (C₃-C₃₀)aliphatic ring(s) and a (C₆-C₃₀)aromatic ring(s), amino, mono- or di(C₁-C₃₀)alkylamino, mono- or di(C₆-C₃₀)arylamino, (C₁-C₃₀)alkyl(C₆-C₃₀)arylamino, mono- or di(3- to 30-membered)heteroarylamino, (C₁-C₃₀)alkyl(3- to 30-membered)heteroarylamino, (C₆-C₃₀)aryl(3- to 30-membered)heteroarylamino, (C₁-C₃₀)alkylcarbonyl, (C₁-C₃₀)alkoxycarbonyl, (C₆-C₃₀)arylcarbonyl, (C₆-C₃₀)arylphosphinyl, di(C₆-C₃₀)arylboronyl, di(C₁-C₃₀)alkylboronyl, (C₁-C₃₀)alkyl(C₆-C₃₀)arylboronyl, (C₆-C₃₀)ar(C₁-C₃₀)alkyl, (C₁-C₃₀)alkyl(C₆-C₃₀)aryl, and a combination thereof. According to one embodiment of the present disclosure, the group may consist of deuterium, (C₁-C₂₀)alkyl, (C₆-C₂₅)aryl, (3- to 25-membered)heteroaryl, and a combination thereof. According to another embodiment of the present disclosure, the group may consist of deuterium, (C₁-C₁₀)alkyl, (C₆-C₁)aryl, (6- to 14-membered)heteroaryl, and a combination thereof. For example, the group may consist of deuterium, methyl, phenyl, naphthyl, biphenyl, pyridyl, pyridyl substituted with pyridyl, etc., which may be further substituted with deuterium.

Herein, if a substituent is not indicated in the formula or compound structure, it may mean that all possible positions for the substituent are hydrogen or deuterium. That is, in the case of deuterium, it is an isotope of hydrogen, and some hydrogen atoms may be the isotope deuterium, and in this case, the content of deuterium may be 0% to 100%. In cases where a substituent is not indicated in the formula or compound structure in the present disclosure, if deuterium is not explicitly excluded, such as 0% deuterium, 100% hydrogen, and all substituents being hydrogen, hydrogen and deuterium may be used together in a compound. Deuterium is one of the isotopes of hydrogen and an element with a deuteron, consisting of one proton and one neutron, as its nucleus. It can be represented as hydrogen-2, whose element symbol can also be written as D or ²H. The isotopes are atoms with the same atomic number (Z) but different mass numbers (A), and can also be interpreted as elements with the same number of protons but different a number of neutrons.

Herein, “a combination thereof” refers to a combination of one or more elements from the corresponding list to form a known or chemically stable arrangement that can be envisioned by one skilled in the art from the corresponding list. For example, alkyl and deuterium can be combined to form a partially or fully deuterated alkyl group; halogen and aryl can be combined to form a halogenated alkyl substituent; and halogen, alkyl, and aryl can be combined to form a halogenated arylalkyl. For example, a preferred combination of substituents comprises up to 50 atoms excluding hydrogen or deuterium, up to 40 atoms excluding hydrogen or deuterium, up to 30 atoms excluding hydrogen or deuterium, or in many cases, a preferred combination of substituents may comprise up to 20 atoms excluding hydrogen or deuterium.

In formulas of the present disclosure, when there are multiple substituents represented by the same symbol, each substituent represented by the same symbol may be the same as or different from one another.

The compound represented by Formula 1 is described in more detail as follows.

In Formula 1, X₁ to X₁₀ each independently represent CR₄ or N. According to one embodiment of the present disclosure, at least one of X₁ to X₁₀ is N. For example, X₁ may be N, or X₅ may be N. According to another embodiment of the present disclosure, two or more of X₁ to X₁₀ are N. According to yet another embodiment of the present disclosure, any one of X₁ to X₅ is N, and any one of X₆ to X₁₀ is N. For example, X₁ or X₅ is N, and X₆ or X₁₀ is N.

In Formula 1, R₁ to R₄ each independently represent hydrogen, deuterium, a halogen, a cyano, a substituted or unsubstituted (C₁-C₃₀)alkyl, a substituted or unsubstituted (C₂-C₃₀)alkenyl, a substituted or unsubstituted (C₃-C₃₀)cycloalkyl, a substituted or unsubstituted (C₃-C₃₀)cycloalkenyl, a substituted or unsubstituted (3- to 7-membered)heterocycloalkyl, or -(L)ₐ-HAr; at least one of R₁ to R₄ is -(L)ₐ-HAr; and when R₂ is -(L)ₐ-HAr, each R₄ independently represents hydrogen, deuterium, or a cyano group. According to one embodiment of the present disclosure, any one of R₁ to R₄ is -(L)ₐ-HAr, and the remaining are each independently hydrogen, deuterium, or cyano. According to another embodiment of the present disclosure, any one of R₁ to R₄ is -(L)ₐ-HAr, and the remaining are each independently hydrogen or deuterium. For example, any one of R₂ and R₄ is -(L)ₐ-HAr.

The each L independently represents a single bond, a substituted or unsubstituted (C₆-C₃₀)arylene, a substituted or unsubstituted (C₂-C₃₀)alkenylene, a substituted or unsubstituted (C₂-C₃₀)alkynylene, or a substituted or unsubstituted (3- to 30-membered)heteroarylene. According to one embodiment of the present disclosure, L is a single bond, a substituted or unsubstituted (C₆-C₁₈)arylene, a substituted or unsubstituted (C₂-C₂₀)alkenylene, a substituted or unsubstituted (C₂-C₂₀)alkynylene, or a substituted or unsubstituted (6- to 14-membered)heteroarylene. According to another embodiment of the present disclosure, L is a single bond, a (C₆-C₁₃)arylene unsubstituted or substituted with deuterium or (C₁-C₃₀)alkyl, a substituted or unsubstituted (C₂-C₁₀)alkynylene, or a (6- to 13-membered)heteroarylene unsubstituted or substituted with deuterium or (C₆-C₃₀)aryl. For example, L may be a single bond, ethynylene, phenylene, a naphthylene, biphenylene, phenanthrenylene, dimethylfluorenylene, dibenzofuranylene, dibenzothiophenylene, quinolylene, pyridylene, etc., which may be substituted with at least one selected from the group consisting of deuterium, phenyl, pyridyl, and a combination thereof.

The a is an integer of 1 or 2, and when a is 2, L may be the same as or different from the other.

The HAr represents a substituted or unsubstituted (3- to 30-membered)heteroaryl comprising at least one nitrogen atom. According to one embodiment of the present disclosure, HAr is a substituted or unsubstituted (5- to 20-membered)heteroaryl comprising at least one nitrogen atom. According to another embodiment of the present disclosure, HAr is a substituted or unsubstituted (6- to 15-membered)heteroaryl comprising one or two nitrogen atoms. Herein, the heteroaryl may be substituted with at least one selected from the group consisting of deuterium, (C₁-C₃₀)alkyl, (C₆-C₃₀)aryl, (3- to 30-membered)heteroaryl, and a combination thereof. For example, HAr may be pyridyl, terpyridyl, quaterpyridyl, triazinyl, quinoxalinyl, quinazolinyl, pyrimidinyl, pyrazinyl, phenanthrolinyl, phenanthrooxazolyl, phenanthrothiazolyl, naphthooxazolyl, naphthothiazolyl, etc., which may be substituted with at least one selected from the group consisting of deuterium, methyl, pyridyl, pyridyl substituted with pyridyl, phenyl, naphthyl, biphenyl, and a combination thereof.

In Formula 1, where R₂ is -(L)ₐ-HAr and HAr is a substituted or unsubstituted phenanthrolinyl, L is linked to any one of positions 3 to 8 of the phenanthrolinyl when L is not a substituted or unsubstituted (C₂-C₃₀)alkenylene or a substituted or unsubstituted (C₂-C₃₀)alkynylene.

The HAr may be represented by any one of the following Formulas 1-1 to 1-5.

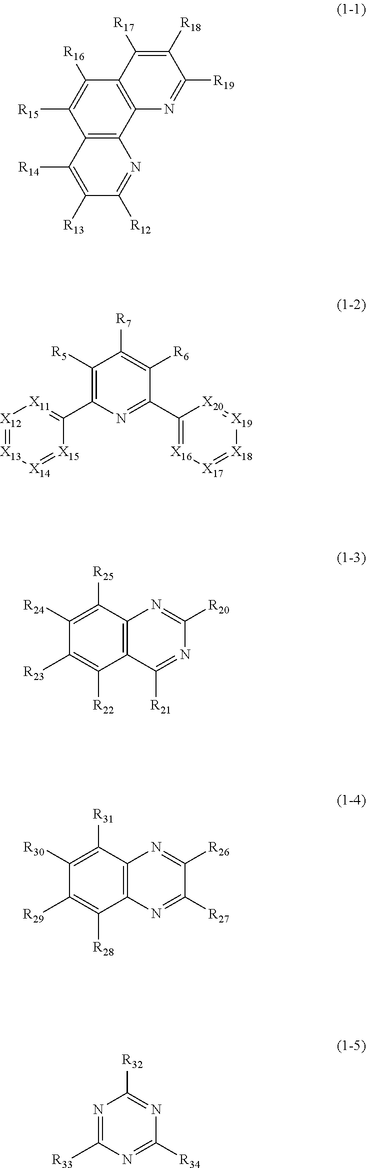

In Formulas 1-1 to 1-5,
X₁₁ to X₂₀ each independently represent CR₈ or N;
R₁₂ and R₁₉ each independently represent hydrogen, deuterium, a halogen, a cyano, a substituted or unsubstituted (C₁-C₃₀)alkyl, a substituted or unsubstituted (C₃-C₃₀)cycloalkyl, a substituted or unsubstituted (C₆-C₃₀)aryl, or a substituted or unsubstituted (3- to 30-membered)heteroaryl;
R₅ to R₈, R₁₃ to R₁₈, and R₂₀ to R₃₄ each independently represent hydrogen, deuterium, a halogen, a cyano, a substituted or unsubstituted (C₁-C₃₀)alkyl, a substituted or unsubstituted (C₃-C₃₀)cycloalkyl, a substituted or unsubstituted (C₆-C₃₀)aryl, a substituted or unsubstituted (3- to 30-membered)heteroaryl, or L;
any one of R₅ to R₈ is L, any one of R₁₃ to R₁₈ is L, any one of R₂₀ to R₂₅ is L, any one of R₂₆ to R₃₁ is L, and any one of R₃₂ to R₃₄ is L; and
L is as defined in Formula 1.

According to one embodiment of the present disclosure, at least one of X₁₁ to X₂₀ is N. According to another embodiment of the present disclosure, two or more of X₁₁ to X₂₀ are N. According to yet another embodiment of the present disclosure, any one of X₁₁ to X₁₅ is N, and any one of X₁₆ to X₂₀ is N. For example, X₁₁ or X₁₅ is N, and X₁₆ or X₂₀ is N.

According to one embodiment of the present disclosure, R₁₂ and R₁₉ are each independently hydrogen, deuterium, a substituted or unsubstituted (C₁-C₁₀)alkyl, or a substituted or unsubstituted (C₆-C₂₅)aryl. According to another embodiment of the present disclosure, R₁₂ and R₁₉ are each independently hydrogen, deuterium, a (C₁-C₆)alkyl unsubstituted or substituted with deuterium, or a (C₆-C₁₈)aryl unsubstituted or substituted with deuterium. For example, R₁₂ and R₁₉ may each independently be hydrogen, deuterium, methyl, phenyl, pyridyl, etc., which may be further substituted with deuterium.

According to one embodiment of the present disclosure, R₅ to R₈, R₁₃ to R₁₈, and R₂₀ to R₃₄ are each independently hydrogen, deuterium, a substituted or unsubstituted (C₆-C₁₈)aryl, or L. According to one embodiment of the present disclosure, R₅ to R₈, R₁₃ to R₁₈, and R₂₀ to R₃₄ are each independently hydrogen, deuterium, a (C₆-C₁₂)aryl unsubstituted or substituted with deuterium, or L. For example, R₅ to R₈, R₁₃ to R₁₈, and R₂₀ to R₃₄ may each independently be L, or hydrogen, deuterium, phenyl, naphthyl, biphenyl, etc., which may be substituted with deuterium.

According to one embodiment of the present disclosure, any one of R₇ and R₈ is L, and any one of R₂₀ and R₂₃ to R₂₅ is L.

The L is as defined in Formula 1.

The -(L)ₐ-HAr may be represented by any one of the following Formulas 1-6 to 1-25.

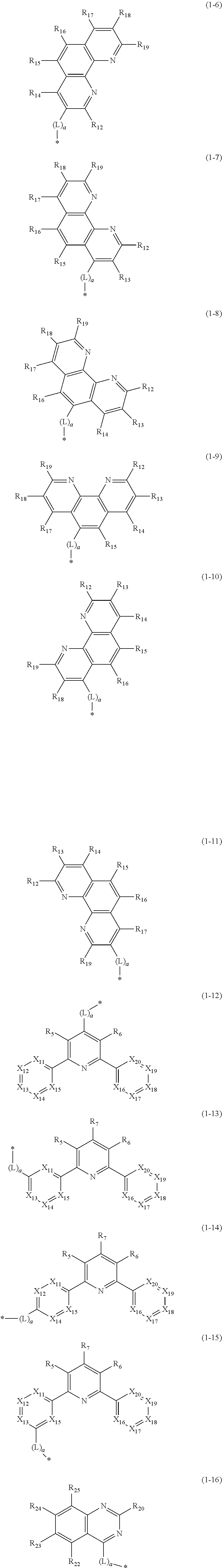

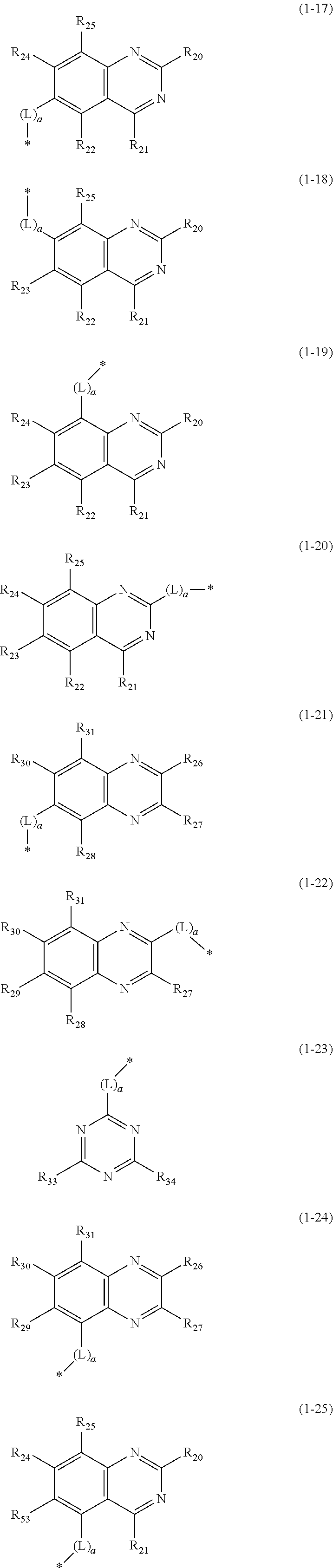

In Formulas 1-6 to 1-25,
X₁₁ to X₂₀ each independently represent CR₈ or N;
R₅ to R₈ and R₁₂ to R₃₄ each independently represent hydrogen, deuterium, a halogen, a cyano, a substituted or unsubstituted (C₁-C₃₀)alkyl, a substituted or unsubstituted (C₃-C₃₀)cycloalkyl, a substituted or unsubstituted (C₆-C₃₀)aryl, or a substituted or unsubstituted (3- to 30-membered)heteroaryl; and
L and a are as defined in Formula 1.

According to one embodiment of the present disclosure, R₅ to R₈ and R₁₂ to R₃₄ are each independently hydrogen, deuterium, a substituted or unsubstituted (C₁-C₁₀)alkyl, or a substituted or unsubstituted (C₆-C₂₅)aryl. According to another embodiment of the present disclosure, R₅ to R₈ and R₁₂ to R₃₄ are each independently hydrogen, deuterium, a (C₁-C₆)alkyl unsubstituted or substituted with deuterium, or a (C₆-C₁₈)aryl unsubstituted or substituted deuterium. For example, R₅ to R₈ and R₁₂ to R₃₄ are each independently hydrogen, deuterium, methyl, phenyl, naphthyl, biphenyl, pyridyl, etc., which may be substituted with deuterium.

The compound represented by Formula 1 may be at least one selected from the following compounds, but is not limited thereto.

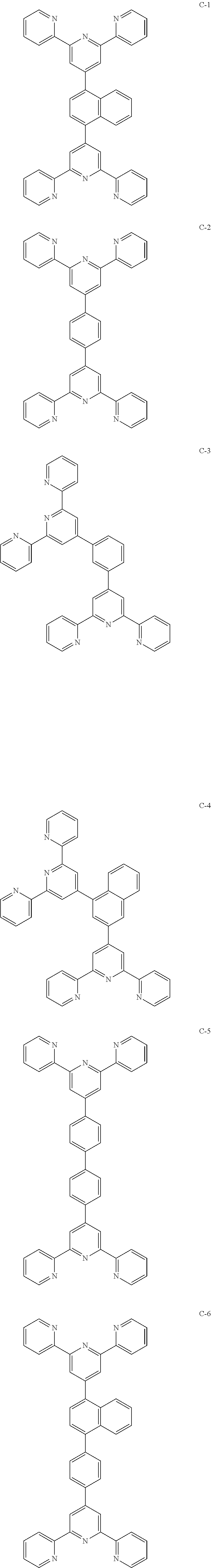 [SMILES: c1ccc(-c2cc(-c3ccc(-c4cc(-c5ccccn5)nc(-c5ccccn5)c4)c4ccccc34)cc(-c3ccccn3)n2)nc1 ; c1ccc(-c2cc(-c3ccc(-c4cc(-c5ccccn5)nc(-c5ccccn5)c4)cc3)cc(-c3ccccn3)n2)nc1 ; c1ccc(-c2cc(-c3cccc(-c4cc(-c5ccccn5)nc(-c5ccccn5)c4)c3)cc(-c3ccccn3)n2)nc1 ; c1ccc(-c2cc(-c3cc(-c4cc(-c5ccccn5)nc(-c5ccccn5)c4)c4ccccc4c3)cc(-c3ccccn3)n2)nc1 ; c1ccc(-c2cc(-c3ccc(-c4ccc(-c5cc(-c6ccccn6)nc(-c6ccccn6)c5)cc4)cc3)cc(-c3ccccn3)n2)nc1 ; c1ccc(-c2cc(-c3ccc(-c4ccc(-c5cc(-c6ccccn6)nc(-c6ccccn6)c5)c5ccccc45)cc3)cc(-c3ccccn3)n2)nc1]

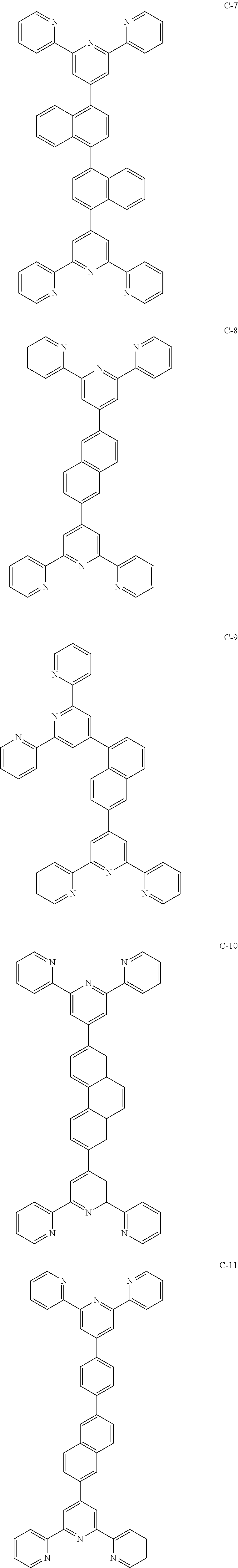

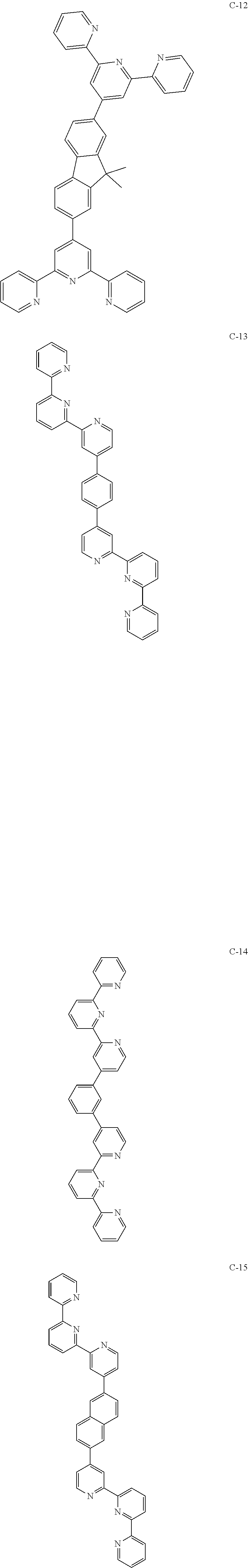 [SMILES: CC1(C)c2cc(-c3cc(-c4ccccn4)nc(-c4ccccn4)c3)ccc2-c2ccc(-c3cc(-c4ccccn4)nc(-c4ccccn4)c3)cc21 ; c1ccc(-c2cccc(-c3cc(-c4ccc(-c5ccnc(-c6cccc(-c7ccccn7)n6)c5)cc4)ccn3)n2)nc1 ; c1ccc(-c2cccc(-c3cc(-c4cccc(-c5ccnc(-c6cccc(-c7ccccn7)n6)c5)c4)ccn3)n2)nc1 ; c1ccc(-c2cccc(-c3cc(-c4ccc5cc(-c6ccnc(-c7cccc(-c8ccccn8)n7)c6)ccc5c4)ccn3)n2)nc1]

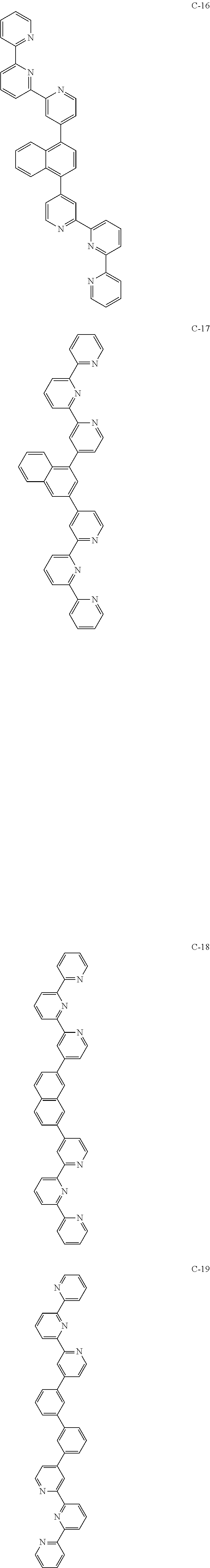 [SMILES: c1ccc(-c2cccc(-c3cc(-c4ccc(-c5ccnc(-c6cccc(-c7ccccn7)n6)c5)c5ccccc45)ccn3)n2)nc1 ; c1ccc(-c2cccc(-c3cc(-c4cc(-c5ccnc(-c6cccc(-c7ccccn7)n6)c5)c5ccccc5c4)ccn3)n2)nc1 ; c1ccc(-c2cccc(-c3cc(-c4ccc5ccc(-c6ccnc(-c7cccc(-c8ccccn8)n7)c6)cc5c4)ccn3)n2)nc1 ; c1ccc(-c2cccc(-c3cc(-c4cccc(-c5cccc(-c6ccnc(-c7cccc(-c8ccccn8)n7)c6)c5)c4)ccn3)n2)nc1]

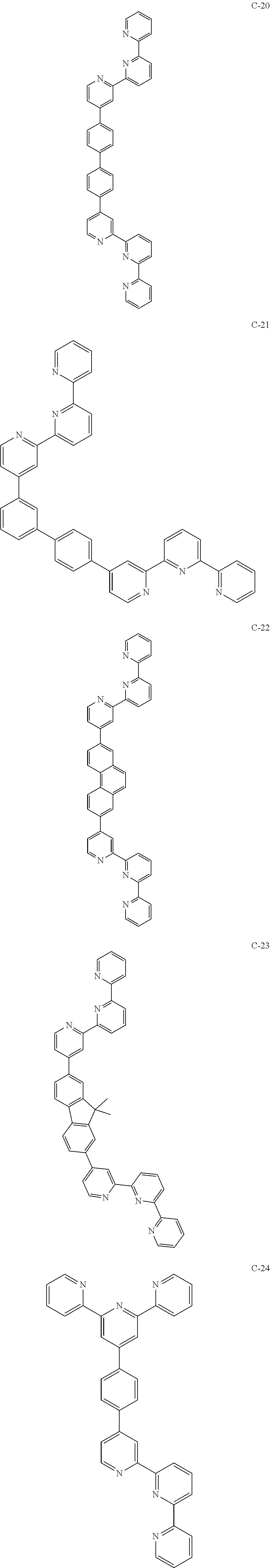 [SMILES: c1ccc(-c2cccc(-c3cc(-c4ccc(-c5ccc(-c6ccnc(-c7cccc(-c8ccccn8)n7)c6)cc5)cc4)ccn3)n2)nc1 ; c1ccc(-c2cccc(-c3cc(-c4ccc(-c5cccc(-c6ccnc(-c7cccc(-c8ccccn8)n7)c6)c5)cc4)ccn3)n2)nc1 ; c1ccc(-c2cccc(-c3cc(-c4ccc5c(ccc6cc(-c7ccnc(-c8cccc(-c9ccccn9)n8)c7)ccc65)c4)ccn3)n2)nc1 ; CC1(C)c2cc(-c3ccnc(-c4cccc(-c5ccccn5)n4)c3)ccc2-c2ccc(-c3ccnc(-c4cccc(-c5ccccn5)n4)c3)cc21 ; c1ccc(-c2cccc(-c3cc(-c4ccc(-c5cc(-c6ccccn6)nc(-c6ccccn6)c5)cc4)ccn3)n2)nc1]

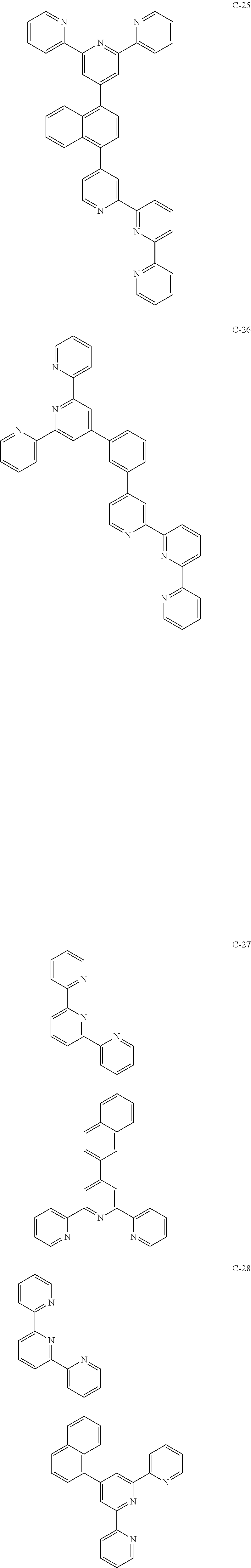 [SMILES: c1ccc(-c2cccc(-c3cc(-c4ccc(-c5cc(-c6ccccn6)nc(-c6ccccn6)c5)c5ccccc45)ccn3)n2)nc1 ; c1ccc(-c2cccc(-c3cc(-c4cccc(-c5cc(-c6ccccn6)nc(-c6ccccn6)c5)c4)ccn3)n2)nc1 ; c1ccc(-c2cccc(-c3cc(-c4ccc5cc(-c6cc(-c7ccccn7)nc(-c7ccccn7)c6)ccc5c4)ccn3)n2)nc1 ; c1ccc(-c2cccc(-c3cc(-c4ccc5c(-c6cc(-c7ccccn7)nc(-c7ccccn7)c6)cccc5c4)ccn3)n2)nc1]

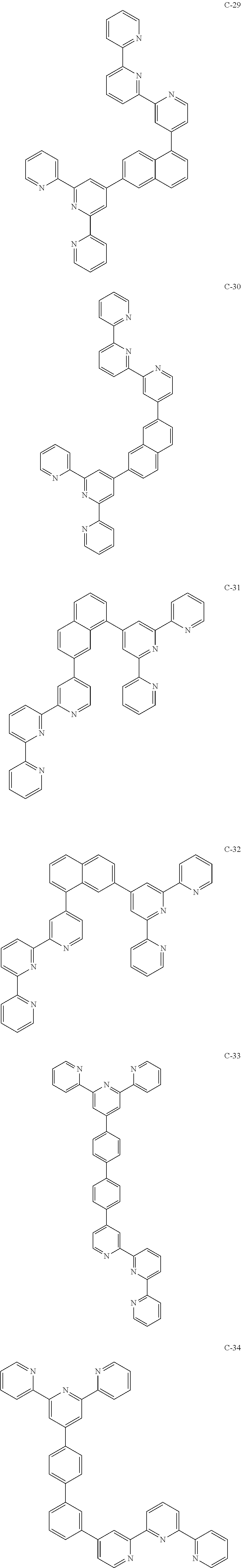

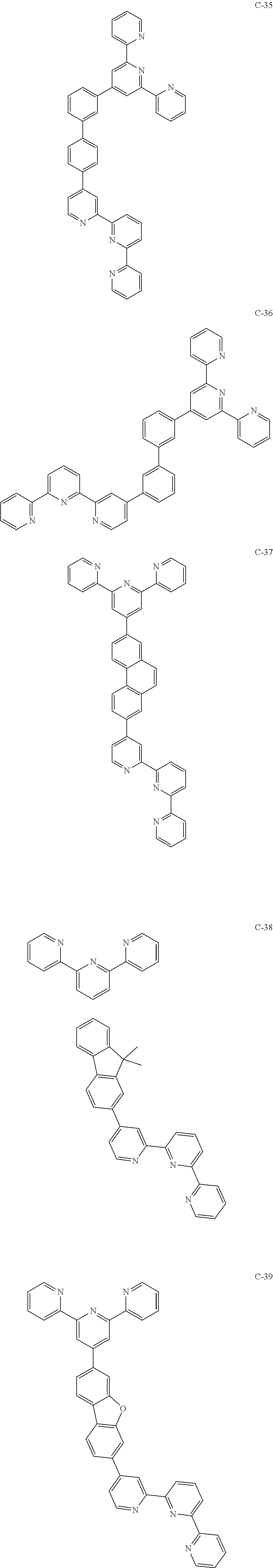

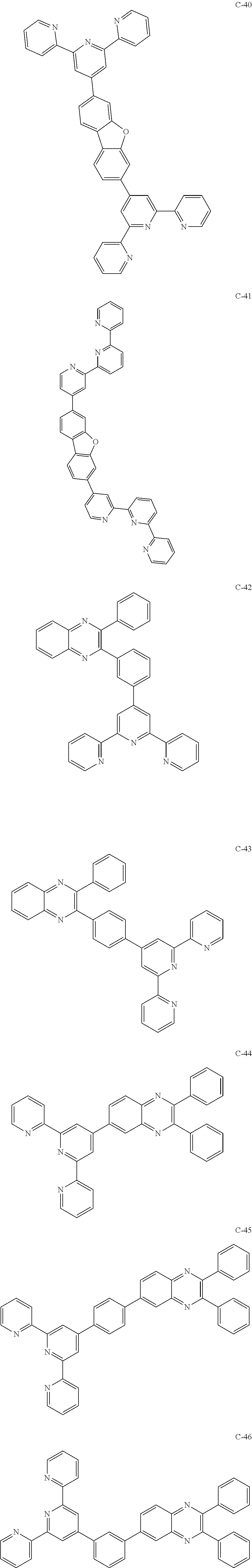

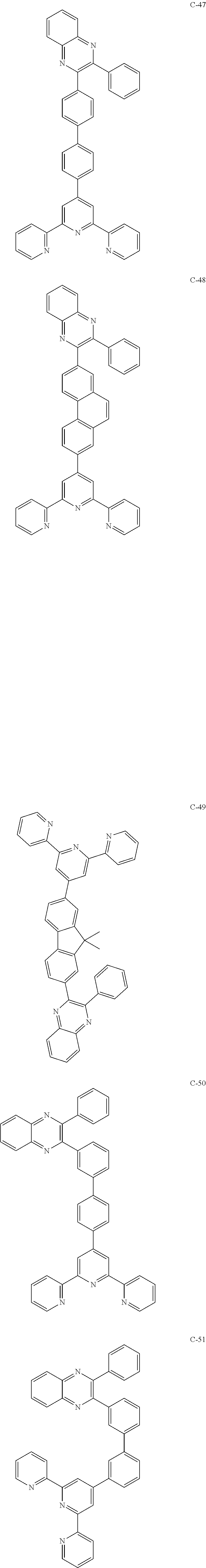 [SMILES: c1ccc(-c2nc3ccccc3nc2-c2ccc(-c3ccc(-c4cc(-c5ccccn5)nc(-c5ccccn5)c4)cc3)cc2)cc1 ; c1ccc(-c2nc3ccccc3nc2-c2ccc3c(ccc4cc(-c5cc(-c6ccccn6)nc(-c6ccccn6)c5)ccc43)c2)cc1 ; CC1(C)c2cc(-c3cc(-c4ccccn4)nc(-c4ccccn4)c3)ccc2-c2ccc(-c3nc4ccccc4nc3-c3ccccc3)cc21 ; c1ccc(-c2nc3ccccc3nc2-c2cccc(-c3ccc(-c4cc(-c5ccccn5)nc(-c5ccccn5)c4)cc3)c2)cc1 ; c1ccc(-c2nc3ccccc3nc2-c2cccc(-c3cccc(-c4cc(-c5ccccn5)nc(-c5ccccn5)c4)c3)c2)cc1]

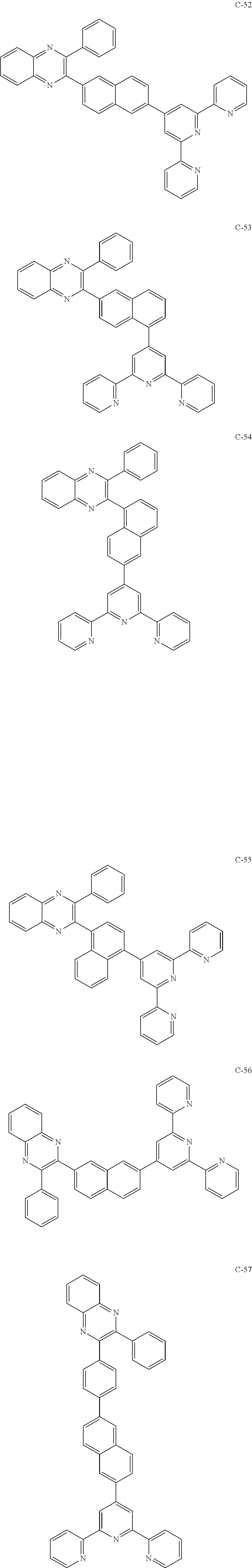 [SMILES: c1ccc(-c2nc3ccccc3nc2-c2ccc3cc(-c4cc(-c5ccccn5)nc(-c5ccccn5)c4)ccc3c2)cc1 ; c1ccc(-c2nc3ccccc3nc2-c2ccc3c(-c4cc(-c5ccccn5)nc(-c5ccccn5)c4)cccc3c2)cc1 ; c1ccc(-c2nc3ccccc3nc2-c2cccc3cc(-c4cc(-c5ccccn5)nc(-c5ccccn5)c4)ccc23)cc1 ; c1ccc(-c2nc3ccccc3nc2-c2ccc(-c3cc(-c4ccccn4)nc(-c4ccccn4)c3)c3ccccc23)cc1 ; c1ccc(-c2nc3ccccc3nc2-c2ccc3ccc(-c4cc(-c5ccccn5)nc(-c5ccccn5)c4)cc3c2)cc1 ; c1ccc(-c2nc3ccccc3nc2-c2ccc(-c3ccc4cc(-c5cc(-c6ccccn6)nc(-c6ccccn6)c5)ccc4c3)cc2)cc1]

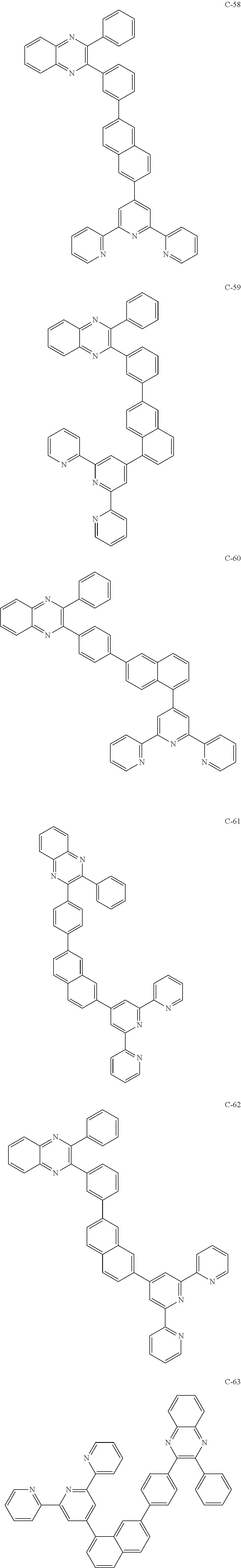

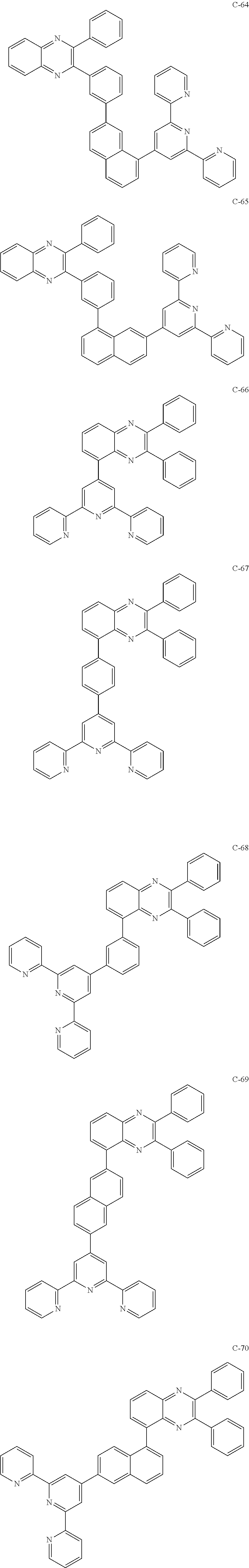

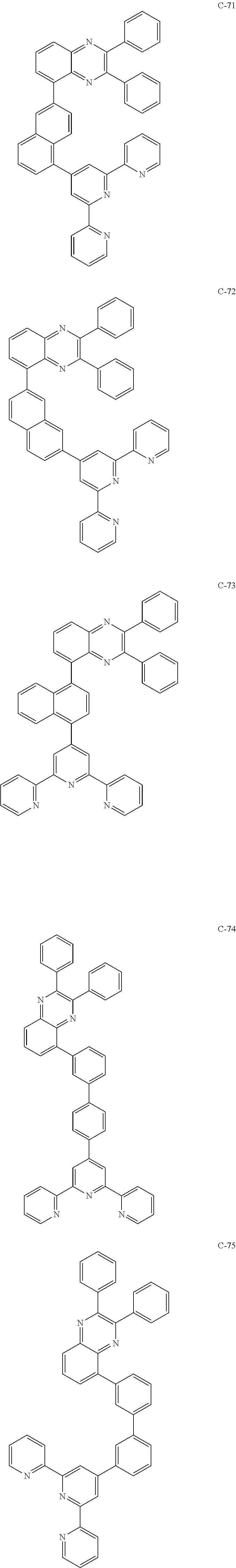 [SMILES: c1ccc(-c2nc3cccc(-c4ccc5c(-c6cc(-c7ccccn7)nc(-c7ccccn7)c6)cccc5c4)c3nc2-c2ccccc2)cc1 ; c1ccc(-c2nc3cccc(-c4ccc5ccc(-c6cc(-c7ccccn7)nc(-c7ccccn7)c6)cc5c4)c3nc2-c2ccccc2)cc1 ; c1ccc(-c2nc3cccc(-c4ccc(-c5cc(-c6ccccn6)nc(-c6ccccn6)c5)c5ccccc45)c3nc2-c2ccccc2)cc1 ; c1ccc(-c2nc3cccc(-c4cccc(-c5ccc(-c6cc(-c7ccccn7)nc(-c7ccccn7)c6)cc5)c4)c3nc2-c2ccccc2)cc1 ; c1ccc(-c2nc3cccc(-c4cccc(-c5cccc(-c6cc(-c7ccccn7)nc(-c7ccccn7)c6)c5)c4)c3nc2-c2ccccc2)cc1]

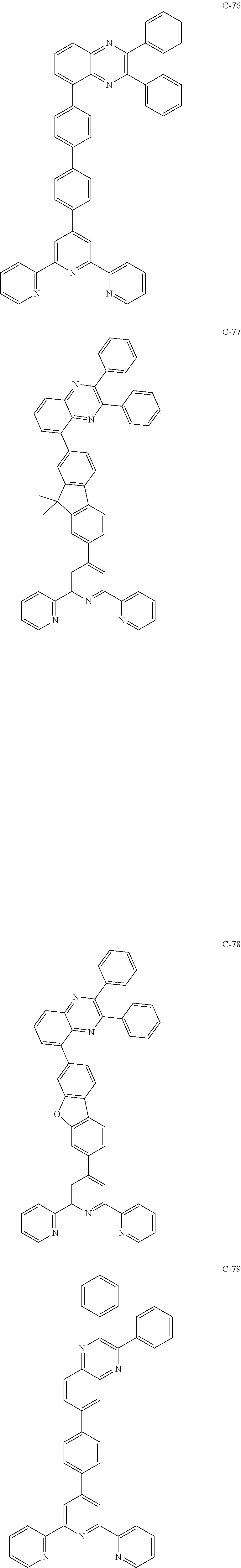 [SMILES: c1ccc(-c2nc3cccc(-c4ccc(-c5ccc(-c6cc(-c7ccccn7)nc(-c7ccccn7)c6)cc5)cc4)c3nc2-c2ccccc2)cc1 ; CC1(C)c2cc(-c3cc(-c4ccccn4)nc(-c4ccccn4)c3)ccc2-c2ccc(-c3cccc4nc(-c5ccccc5)c(-c5ccccc5)nc34)cc21 ; c1ccc(-c2nc3cccc(-c4ccc5c(c4)oc4cc(-c6cc(-c7ccccn7)nc(-c7ccccn7)c6)ccc45)c3nc2-c2ccccc2)cc1 ; c1ccc(-c2nc3ccc(-c4ccc(-c5cc(-c6ccccn6)nc(-c6ccccn6)c5)cc4)cc3nc2-c2ccccc2)cc1]

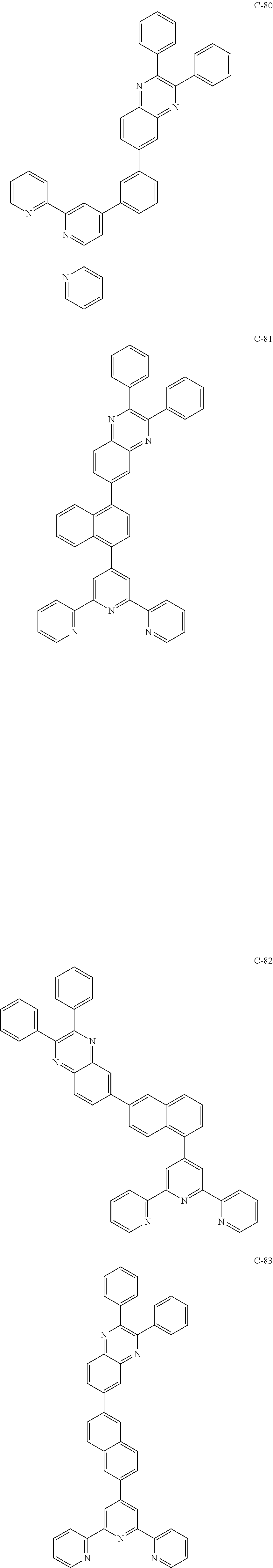 [SMILES: c1ccc(-c2nc3ccc(-c4cccc(-c5cc(-c6ccccn6)nc(-c6ccccn6)c5)c4)cc3nc2-c2ccccc2)cc1 ; c1ccc(-c2nc3ccc(-c4ccc(-c5cc(-c6ccccn6)nc(-c6ccccn6)c5)c5ccccc45)cc3nc2-c2ccccc2)cc1 ; c1ccc(-c2nc3ccc(-c4ccc5c(-c6cc(-c7ccccn7)nc(-c7ccccn7)c6)cccc5c4)cc3nc2-c2ccccc2)cc1 ; c1ccc(-c2nc3ccc(-c4ccc5cc(-c6cc(-c7ccccn7)nc(-c7ccccn7)c6)ccc5c4)cc3nc2-c2ccccc2)cc1]

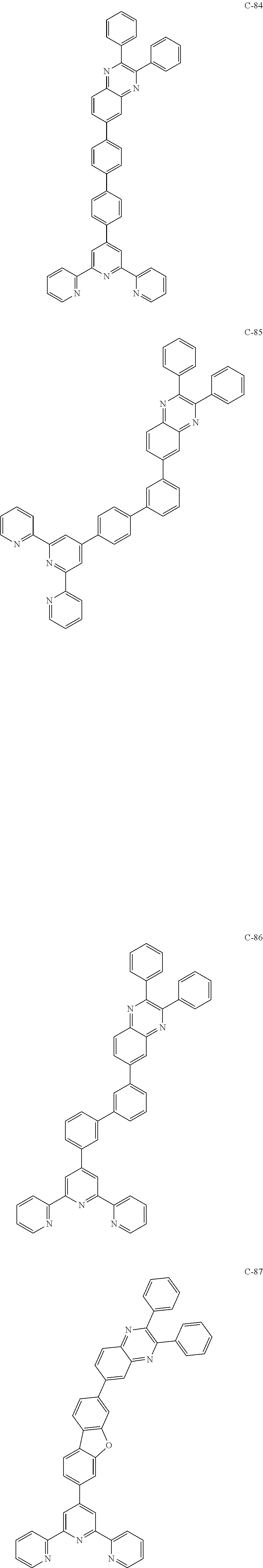 [SMILES: c1ccc(-c2nc3ccc(-c4ccc(-c5ccc(-c6cc(-c7ccccn7)nc(-c7ccccn7)c6)cc5)cc4)cc3nc2-c2ccccc2)cc1 ; c1ccc(-c2nc3ccc(-c4cccc(-c5ccc(-c6cc(-c7ccccn7)nc(-c7ccccn7)c6)cc5)c4)cc3nc2-c2ccccc2)cc1 ; c1ccc(-c2nc3ccc(-c4cccc(-c5cccc(-c6cc(-c7ccccn7)nc(-c7ccccn7)c6)c5)c4)cc3nc2-c2ccccc2)cc1 ; c1ccc(-c2nc3ccc(-c4ccc5c(c4)oc4cc(-c6cc(-c7ccccn7)nc(-c7ccccn7)c6)ccc45)cc3nc2-c2ccccc2)cc1]

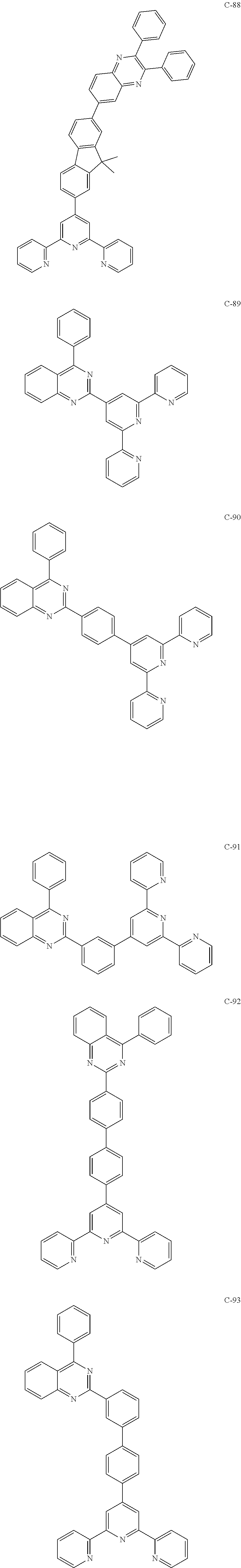 [SMILES: CC1(C)c2cc(-c3cc(-c4ccccn4)nc(-c4ccccn4)c3)ccc2-c2ccc(-c3ccc4nc(-c5ccccc5)c(-c5ccccc5)nc4c3)cc21 ; c1ccc(-c2nc(-c3cc(-c4ccccn4)nc(-c4ccccn4)c3)nc3ccccc23)cc1 ; c1ccc(-c2nc(-c3ccc(-c4cc(-c5ccccn5)nc(-c5ccccn5)c4)cc3)nc3ccccc23)cc1 ; c1ccc(-c2nc(-c3cccc(-c4cc(-c5ccccn5)nc(-c5ccccn5)c4)c3)nc3ccccc23)cc1 ; c1ccc(-c2nc(-c3ccc(-c4ccc(-c5cc(-c6ccccn6)nc(-c6ccccn6)c5)cc4)cc3)nc3ccccc23)cc1 ; c1ccc(-c2nc(-c3cccc(-c4ccc(-c5cc(-c6ccccn6)nc(-c6ccccn6)c5)cc4)c3)nc3ccccc23)cc1]

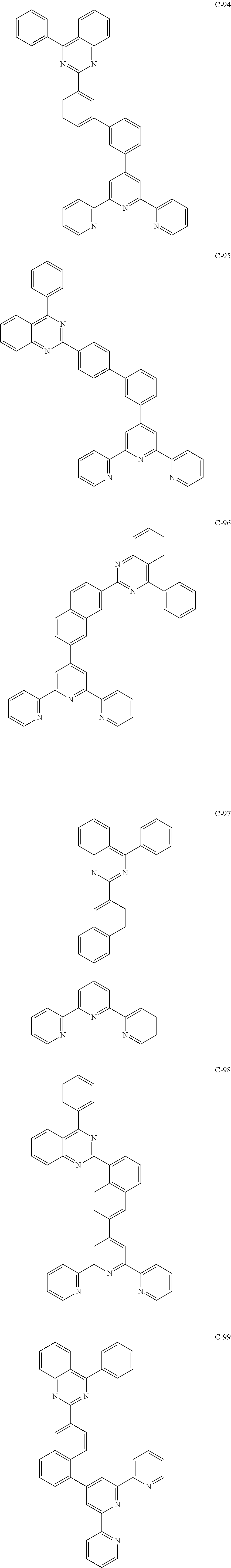 [SMILES: c1ccc(-c2nc(-c3cccc(-c4cccc(-c5cc(-c6ccccn6)nc(-c6ccccn6)c5)c4)c3)nc3ccccc23)cc1 ; c1ccc(-c2nc(-c3ccc(-c4cccc(-c5cc(-c6ccccn6)nc(-c6ccccn6)c5)c4)cc3)nc3ccccc23)cc1 ; c1ccc(-c2nc(-c3ccc4ccc(-c5cc(-c6ccccn6)nc(-c6ccccn6)c5)cc4c3)nc3ccccc23)cc1 ; c1ccc(-c2nc(-c3ccc4cc(-c5cc(-c6ccccn6)nc(-c6ccccn6)c5)ccc4c3)nc3ccccc23)cc1 ; c1ccc(-c2nc(-c3cccc4cc(-c5cc(-c6ccccn6)nc(-c6ccccn6)c5)ccc34)nc3ccccc23)cc1 ; c1ccc(-c2nc(-c3ccc4c(-c5cc(-c6ccccn6)nc(-c6ccccn6)c5)cccc4c3)nc3ccccc23)cc1]

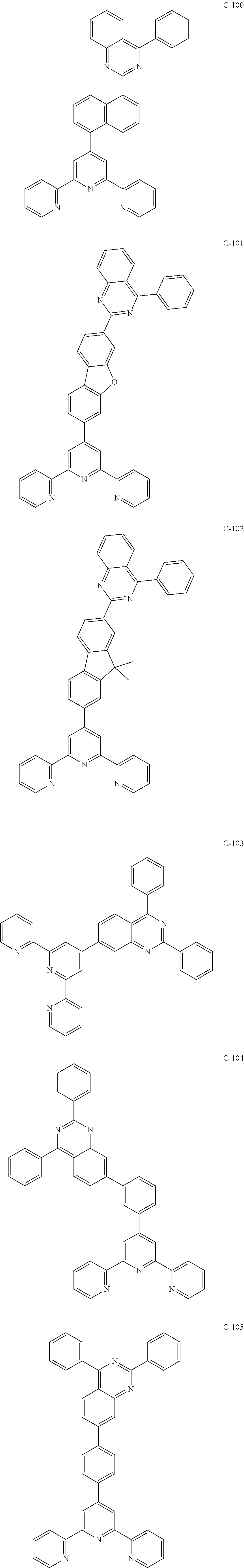 [SMILES: c1ccc(-c2nc(-c3cccc4c(-c5cc(-c6ccccn6)nc(-c6ccccn6)c5)cccc34)nc3ccccc23)cc1 ; c1ccc(-c2nc(-c3ccc4c(c3)oc3cc(-c5cc(-c6ccccn6)nc(-c6ccccn6)c5)ccc34)nc3ccccc23)cc1 ; CC1(C)c2cc(-c3cc(-c4ccccn4)nc(-c4ccccn4)c3)ccc2-c2ccc(-c3nc(-c4ccccc4)c4ccccc4n3)cc21 ; c1ccc(-c2nc(-c3ccccc3)c3ccc(-c4cc(-c5ccccn5)nc(-c5ccccn5)c4)cc3n2)cc1 ; c1ccc(-c2nc(-c3ccccc3)c3ccc(-c4cccc(-c5cc(-c6ccccn6)nc(-c6ccccn6)c5)c4)cc3n2)cc1 ; c1ccc(-c2nc(-c3ccccc3)c3ccc(-c4ccc(-c5cc(-c6ccccn6)nc(-c6ccccn6)c5)cc4)cc3n2)cc1]

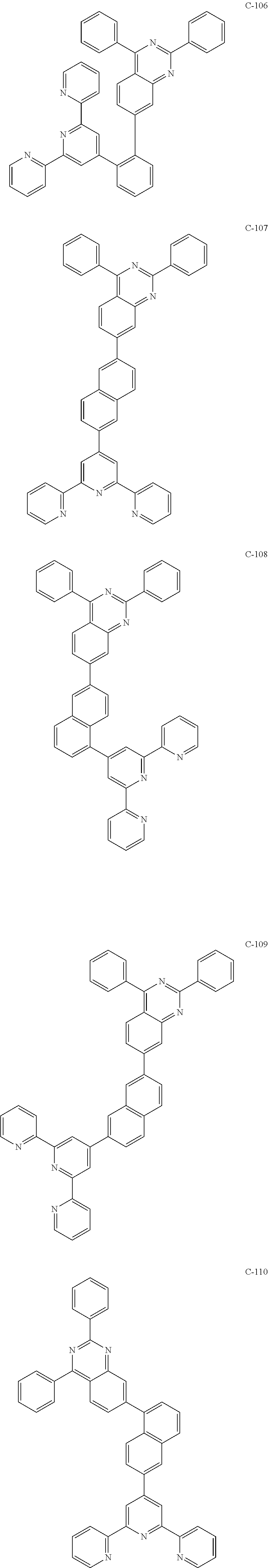 [SMILES: c1ccc(-c2nc(-c3ccccc3)c3ccc(-c4ccccc4-c4cc(-c5ccccn5)nc(-c5ccccn5)c4)cc3n2)cc1 ; c1ccc(-c2nc(-c3ccccc3)c3ccc(-c4ccc5cc(-c6cc(-c7ccccn7)nc(-c7ccccn7)c6)ccc5c4)cc3n2)cc1 ; c1ccc(-c2nc(-c3ccccc3)c3ccc(-c4ccc5c(-c6cc(-c7ccccn7)nc(-c7ccccn7)c6)cccc5c4)cc3n2)cc1 ; c1ccc(-c2nc(-c3ccccc3)c3ccc(-c4ccc5ccc(-c6cc(-c7ccccn7)nc(-c7ccccn7)c6)cc5c4)cc3n2)cc1 ; c1ccc(-c2nc(-c3ccccc3)c3ccc(-c4cccc5cc(-c6cc(-c7ccccn7)nc(-c7ccccn7)c6)ccc45)cc3n2)cc1]

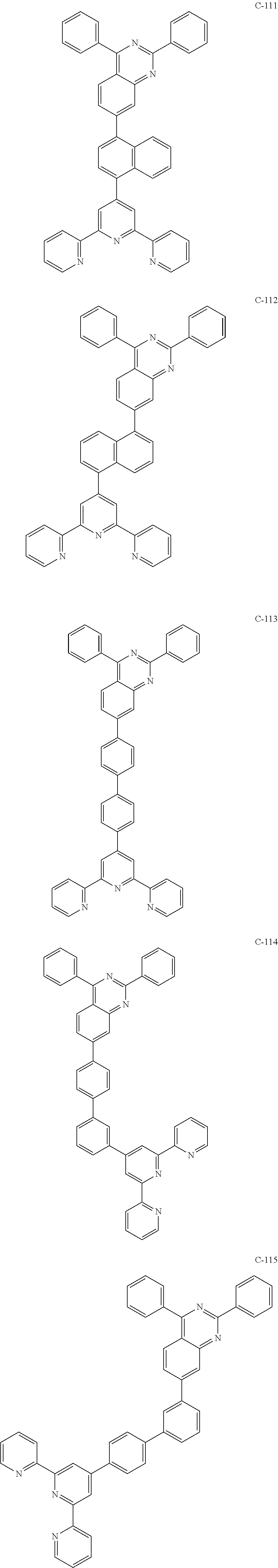 [SMILES: c1ccc(-c2nc(-c3ccccc3)c3ccc(-c4ccc(-c5cc(-c6ccccn6)nc(-c6ccccn6)c5)c5ccccc45)cc3n2)cc1 ; c1ccc(-c2nc(-c3ccccc3)c3ccc(-c4cccc5c(-c6cc(-c7ccccn7)nc(-c7ccccn7)c6)cccc45)cc3n2)cc1 ; c1ccc(-c2nc(-c3ccccc3)c3ccc(-c4ccc(-c5ccc(-c6cc(-c7ccccn7)nc(-c7ccccn7)c6)cc5)cc4)cc3n2)cc1 ; c1ccc(-c2nc(-c3ccccc3)c3ccc(-c4ccc(-c5cccc(-c6cc(-c7ccccn7)nc(-c7ccccn7)c6)c5)cc4)cc3n2)cc1 ; c1ccc(-c2nc(-c3ccccc3)c3ccc(-c4cccc(-c5ccc(-c6cc(-c7ccccn7)nc(-c7ccccn7)c6)cc5)c4)cc3n2)cc1]

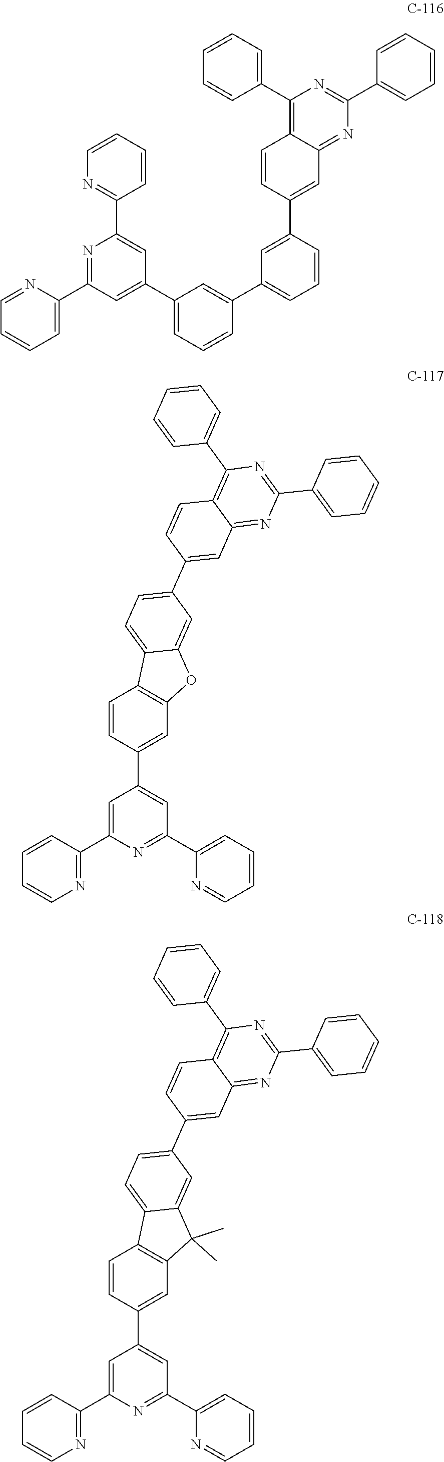

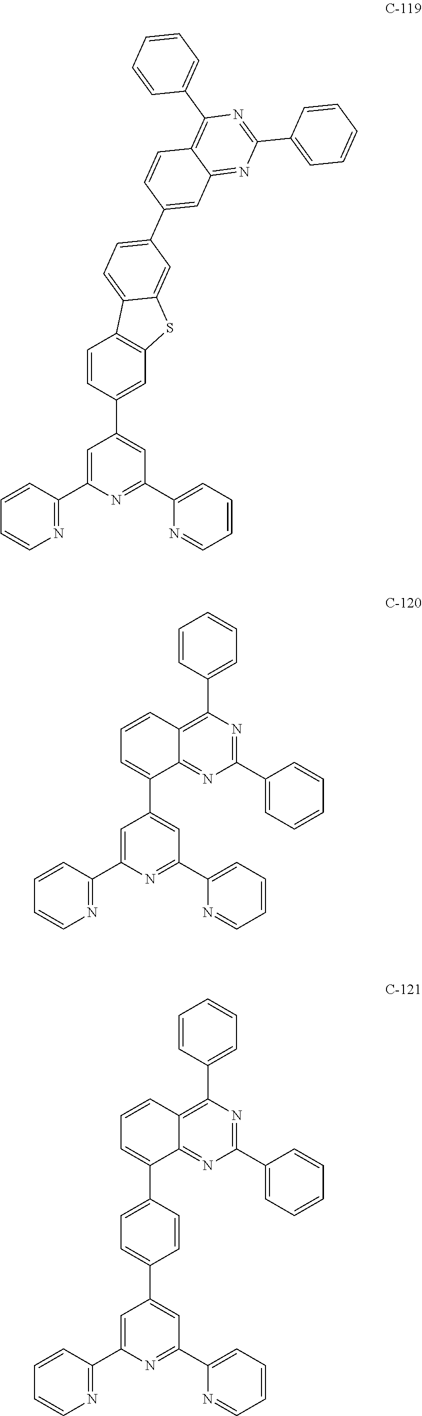 [SMILES: c1ccc(-c2nc(-c3ccccc3)c3ccc(-c4ccc5c(c4)sc4cc(-c6cc(-c7ccccn7)nc(-c7ccccn7)c6)ccc45)cc3n2)cc1 ; c1ccc(-c2nc(-c3ccccc3)c3cccc(-c4cc(-c5ccccn5)nc(-c5ccccn5)c4)c3n2)cc1 ; c1ccc(-c2nc(-c3ccccc3)c3cccc(-c4ccc(-c5cc(-c6ccccn6)nc(-c6ccccn6)c5)cc4)c3n2)cc1]

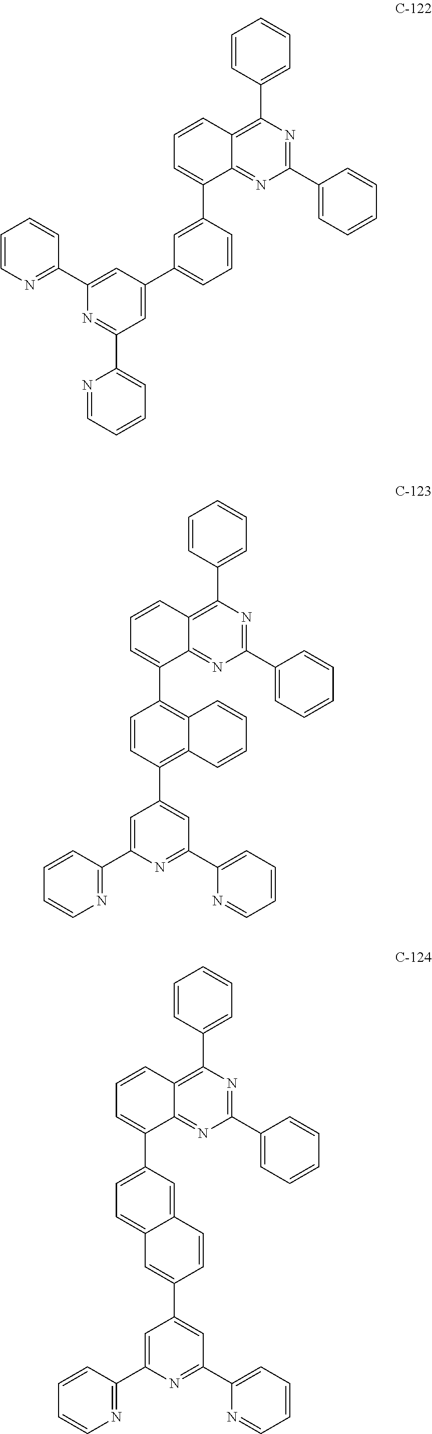 [SMILES: c1ccc(-c2nc(-c3ccccc3)c3cccc(-c4cccc(-c5cc(-c6ccccn6)nc(-c6ccccn6)c5)c4)c3n2)cc1 ; c1ccc(-c2nc(-c3ccccc3)c3cccc(-c4ccc(-c5cc(-c6ccccn6)nc(-c6ccccn6)c5)c5ccccc45)c3n2)cc1 ; c1ccc(-c2nc(-c3ccccc3)c3cccc(-c4ccc5cc(-c6cc(-c7ccccn7)nc(-c7ccccn7)c6)ccc5c4)c3n2)cc1]

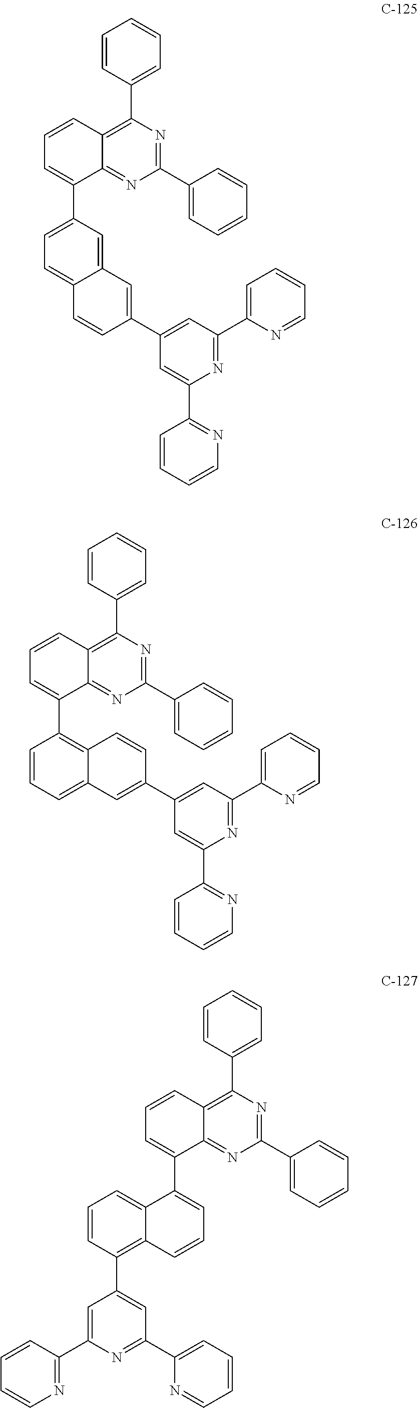 [SMILES: c1ccc(-c2nc(-c3ccccc3)c3cccc(-c4ccc5ccc(-c6cc(-c7ccccn7)nc(-c7ccccn7)c6)cc5c4)c3n2)cc1 ; c1ccc(-c2nc(-c3ccccc3)c3cccc(-c4cccc5cc(-c6cc(-c7ccccn7)nc(-c7ccccn7)c6)ccc45)c3n2)cc1 ; c1ccc(-c2nc(-c3ccccc3)c3cccc(-c4cccc5c(-c6cc(-c7ccccn7)nc(-c7ccccn7)c6)cccc45)c3n2)cc1]

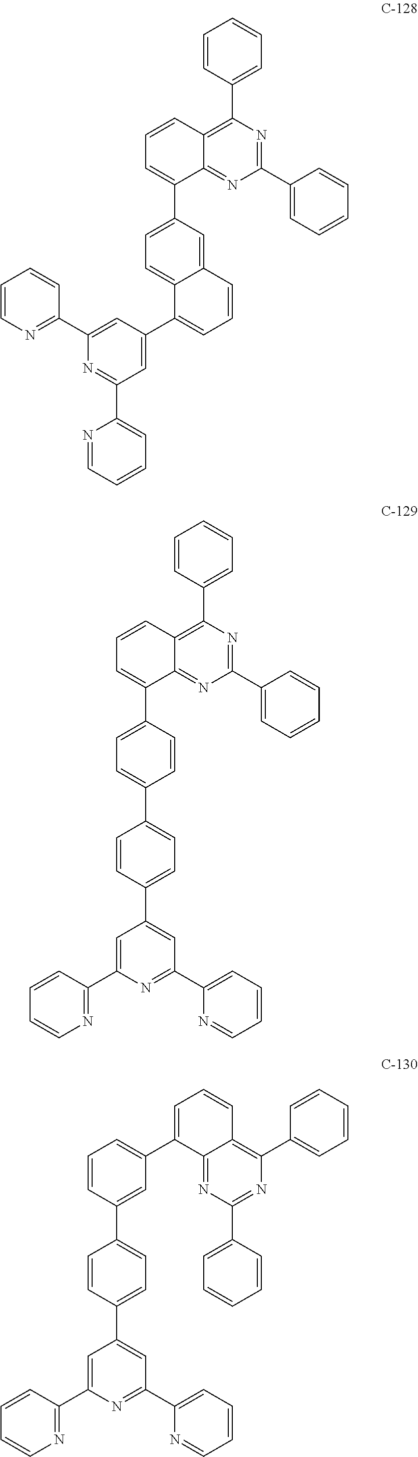 [SMILES: c1ccc(-c2nc(-c3ccccc3)c3cccc(-c4ccc5c(-c6cc(-c7ccccn7)nc(-c7ccccn7)c6)cccc5c4)c3n2)cc1 ; c1ccc(-c2nc(-c3ccccc3)c3cccc(-c4ccc(-c5ccc(-c6cc(-c7ccccn7)nc(-c7ccccn7)c6)cc5)cc4)c3n2)cc1 ; c1ccc(-c2nc(-c3ccccc3)c3cccc(-c4cccc(-c5ccc(-c6cc(-c7ccccn7)nc(-c7ccccn7)c6)cc5)c4)c3n2)cc1]

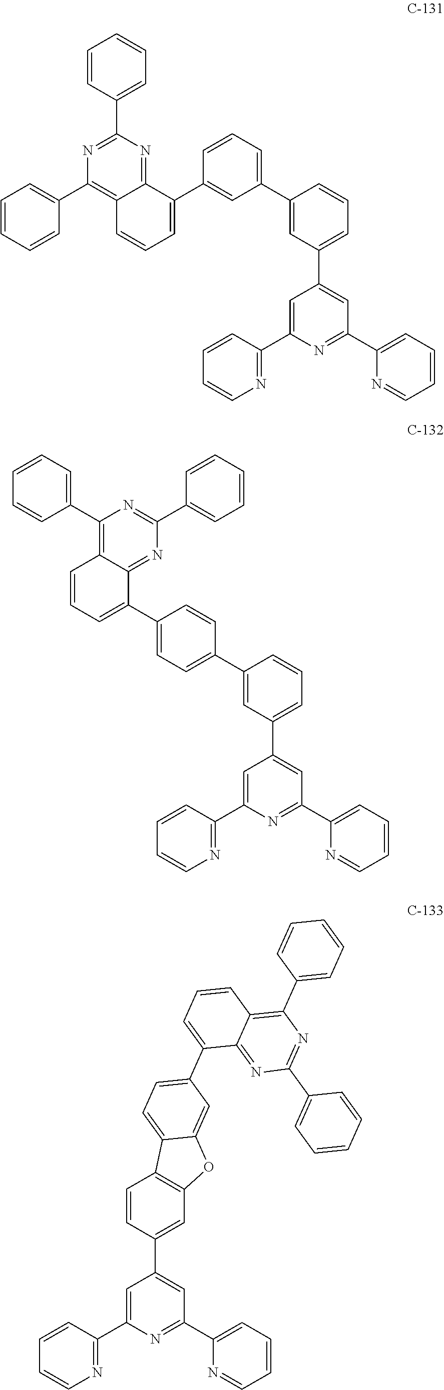 [SMILES: c1ccc(-c2nc(-c3ccccc3)c3cccc(-c4cccc(-c5cccc(-c6cc(-c7ccccn7)nc(-c7ccccn7)c6)c5)c4)c3n2)cc1 ; c1ccc(-c2nc(-c3ccccc3)c3cccc(-c4ccc(-c5cccc(-c6cc(-c7ccccn7)nc(-c7ccccn7)c6)c5)cc4)c3n2)cc1 ; c1ccc(-c2nc(-c3ccccc3)c3cccc(-c4ccc5c(c4)oc4cc(-c6cc(-c7ccccn7)nc(-c7ccccn7)c6)ccc45)c3n2)cc1]

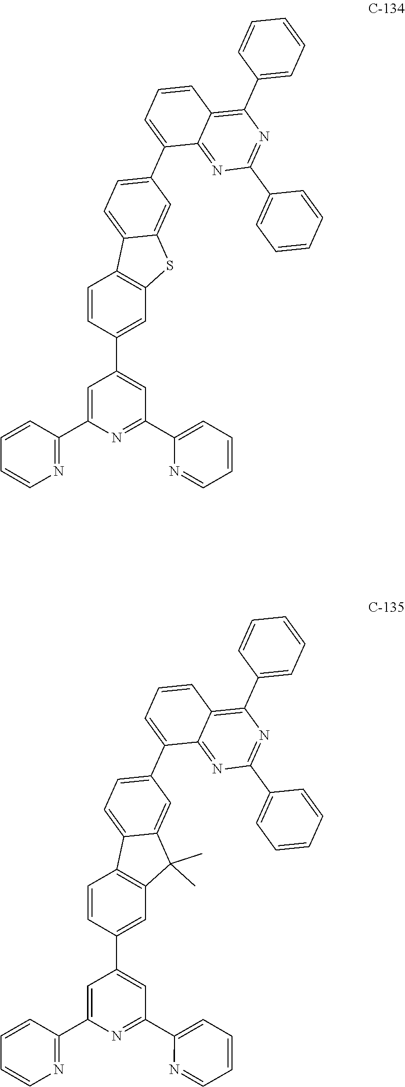 [SMILES: c1ccc(-c2nc(-c3ccccc3)c3cccc(-c4ccc5c(c4)sc4cc(-c6cc(-c7ccccn7)nc(-c7ccccn7)c6)ccc45)c3n2)cc1 ; CC1(C)c2cc(-c3cc(-c4ccccn4)nc(-c4ccccn4)c3)ccc2-c2ccc(-c3cccc4c(-c5ccccc5)nc(-c5ccccc5)nc34)cc21]

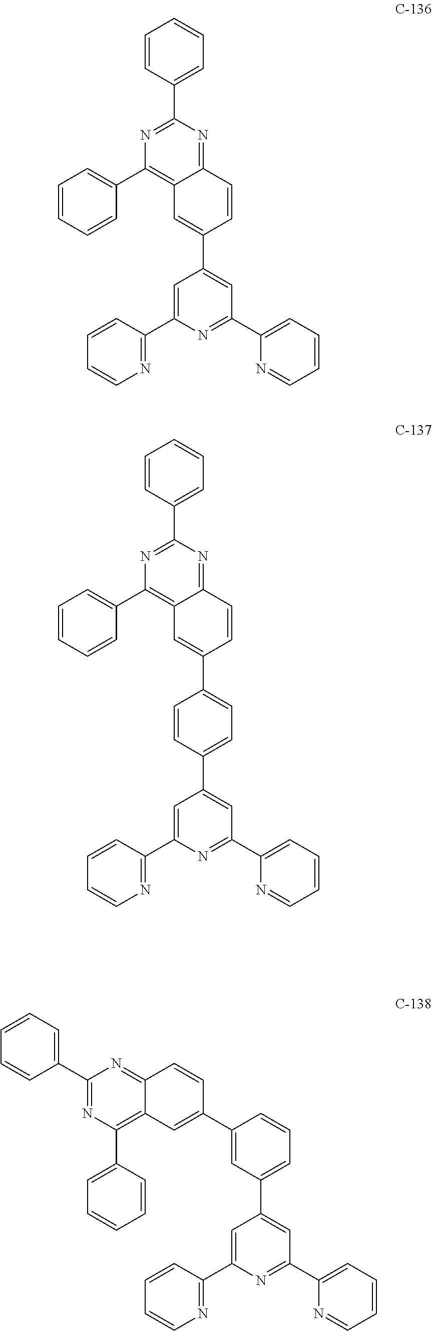 [SMILES: c1ccc(-c2nc(-c3ccccc3)c3cc(-c4cc(-c5ccccn5)nc(-c5ccccn5)c4)ccc3n2)cc1 ; c1ccc(-c2nc(-c3ccccc3)c3cc(-c4ccc(-c5cc(-c6ccccn6)nc(-c6ccccn6)c5)cc4)ccc3n2)cc1 ; c1ccc(-c2nc(-c3ccccc3)c3cc(-c4cccc(-c5cc(-c6ccccn6)nc(-c6ccccn6)c5)c4)ccc3n2)cc1]

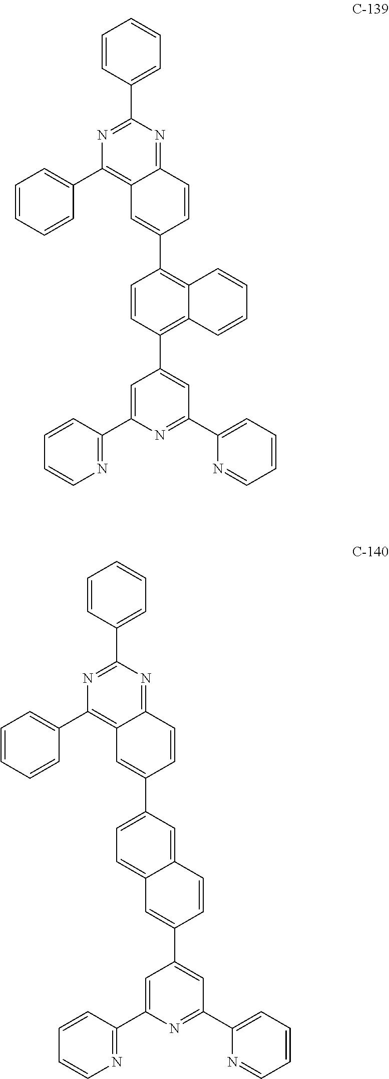 [SMILES: c1ccc(-c2nc(-c3ccccc3)c3cc(-c4ccc(-c5cc(-c6ccccn6)nc(-c6ccccn6)c5)c5ccccc45)ccc3n2)cc1 ; c1ccc(-c2nc(-c3ccccc3)c3cc(-c4ccc5cc(-c6cc(-c7ccccn7)nc(-c7ccccn7)c6)ccc5c4)ccc3n2)cc1]

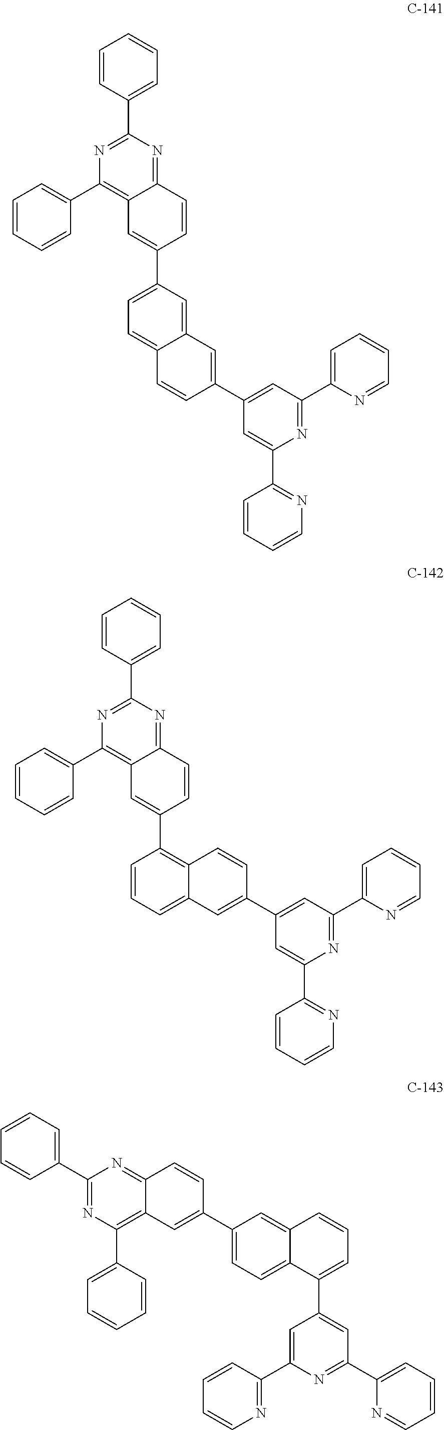 [SMILES: c1ccc(-c2nc(-c3ccccc3)c3cc(-c4ccc5ccc(-c6cc(-c7ccccn7)nc(-c7ccccn7)c6)cc5c4)ccc3n2)cc1 ; c1ccc(-c2nc(-c3ccccc3)c3cc(-c4cccc5cc(-c6cc(-c7ccccn7)nc(-c7ccccn7)c6)ccc45)ccc3n2)cc1 ; c1ccc(-c2nc(-c3ccccc3)c3cc(-c4ccc5c(-c6cc(-c7ccccn7)nc(-c7ccccn7)c6)cccc5c4)ccc3n2)cc1]

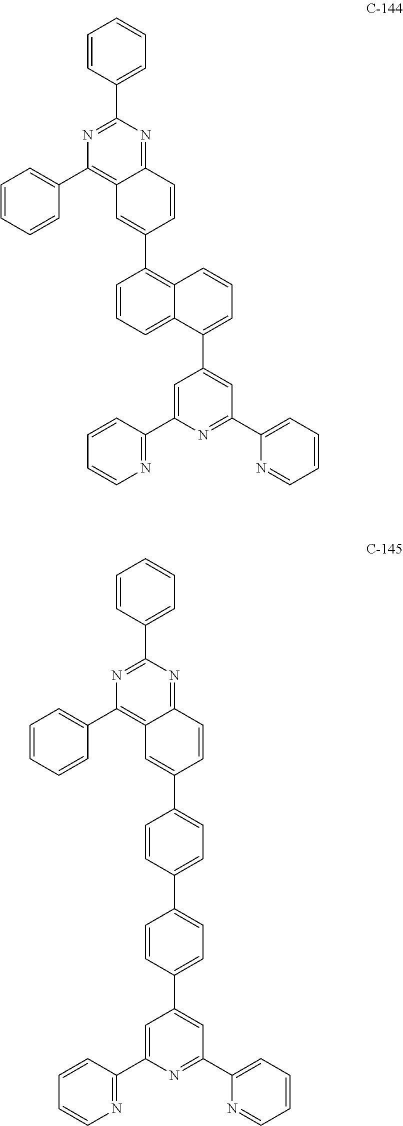 [SMILES: c1ccc(-c2nc(-c3ccccc3)c3cc(-c4cccc5c(-c6cc(-c7ccccn7)nc(-c7ccccn7)c6)cccc45)ccc3n2)cc1 ; c1ccc(-c2nc(-c3ccccc3)c3cc(-c4ccc(-c5ccc(-c6cc(-c7ccccn7)nc(-c7ccccn7)c6)cc5)cc4)ccc3n2)cc1]

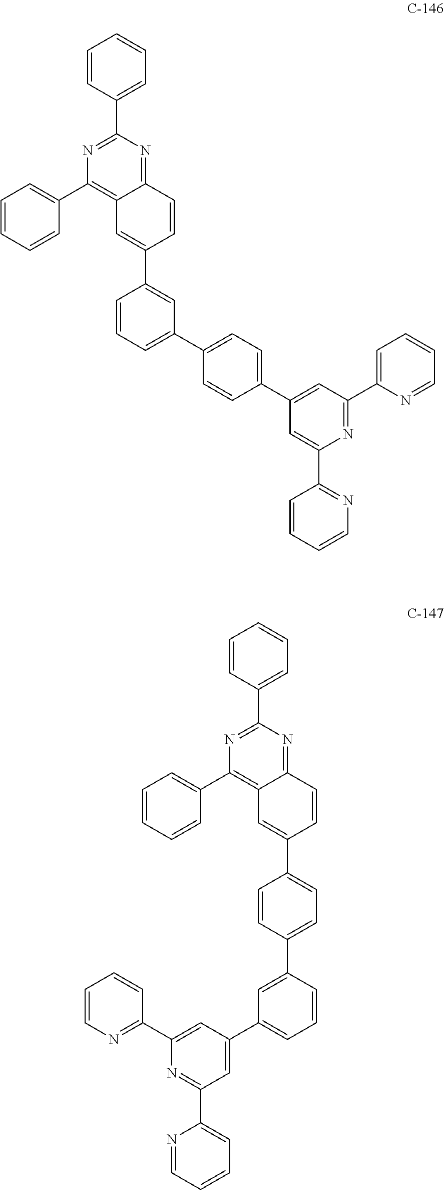 [SMILES: c1ccc(-c2nc(-c3ccccc3)c3cc(-c4cccc(-c5ccc(-c6cc(-c7ccccn7)nc(-c7ccccn7)c6)cc5)c4)ccc3n2)cc1 ; c1ccc(-c2nc(-c3ccccc3)c3cc(-c4ccc(-c5cccc(-c6cc(-c7ccccn7)nc(-c7ccccn7)c6)c5)cc4)ccc3n2)cc1]

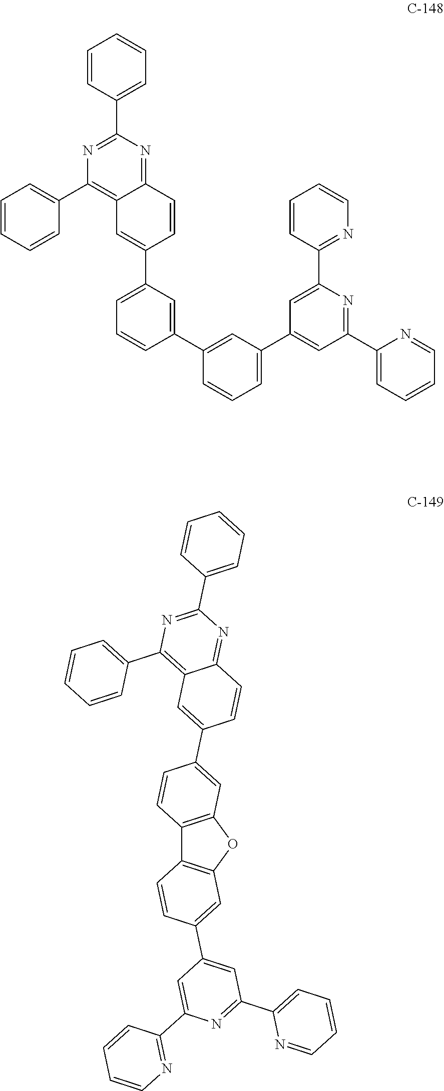 [SMILES: c1ccc(-c2nc(-c3ccccc3)c3cc(-c4cccc(-c5cccc(-c6cc(-c7ccccn7)nc(-c7ccccn7)c6)c5)c4)ccc3n2)cc1 ; c1ccc(-c2nc(-c3ccccc3)c3cc(-c4ccc5c(c4)oc4cc(-c6cc(-c7ccccn7)nc(-c7ccccn7)c6)ccc45)ccc3n2)cc1]

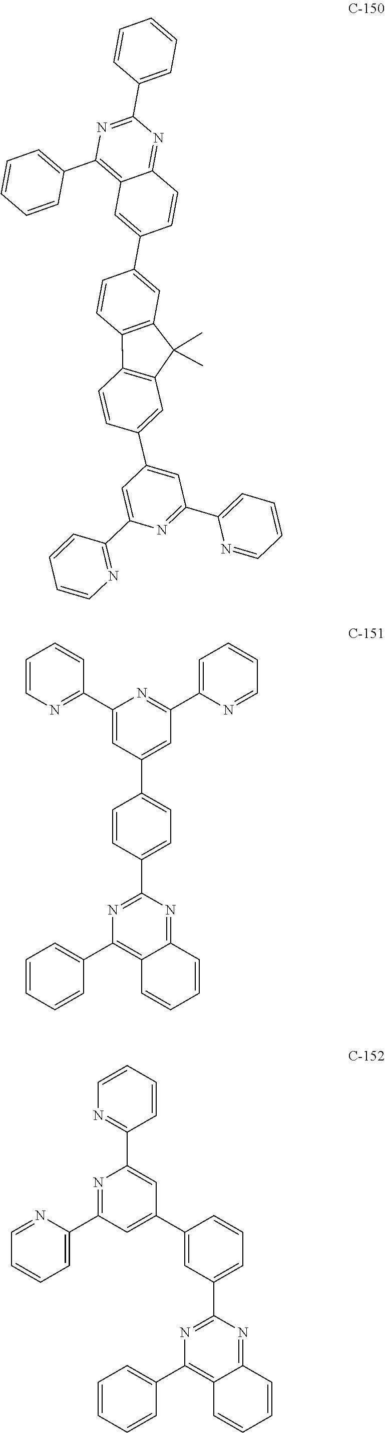 [SMILES: CC1(C)c2cc(-c3cc(-c4ccccn4)nc(-c4ccccn4)c3)ccc2-c2ccc(-c3ccc4nc(-c5ccccc5)nc(-c5ccccc5)c4c3)cc21 ; c1ccc(-c2nc(-c3ccc(-c4cc(-c5ccccn5)nc(-c5ccccn5)c4)cc3)nc3ccccc23)cc1 ; c1ccc(-c2nc(-c3cccc(-c4cc(-c5ccccn5)nc(-c5ccccn5)c4)c3)nc3ccccc23)cc1]

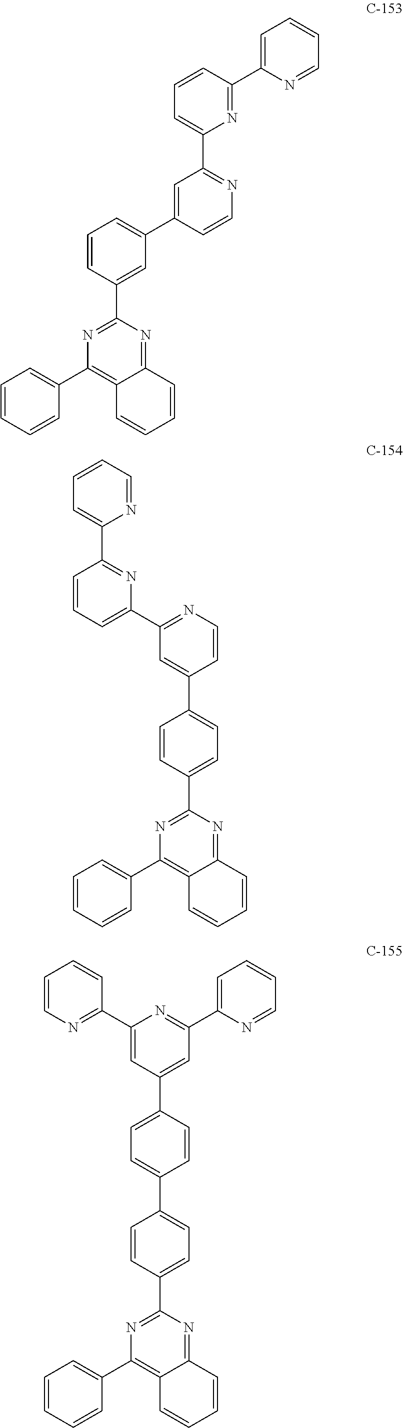

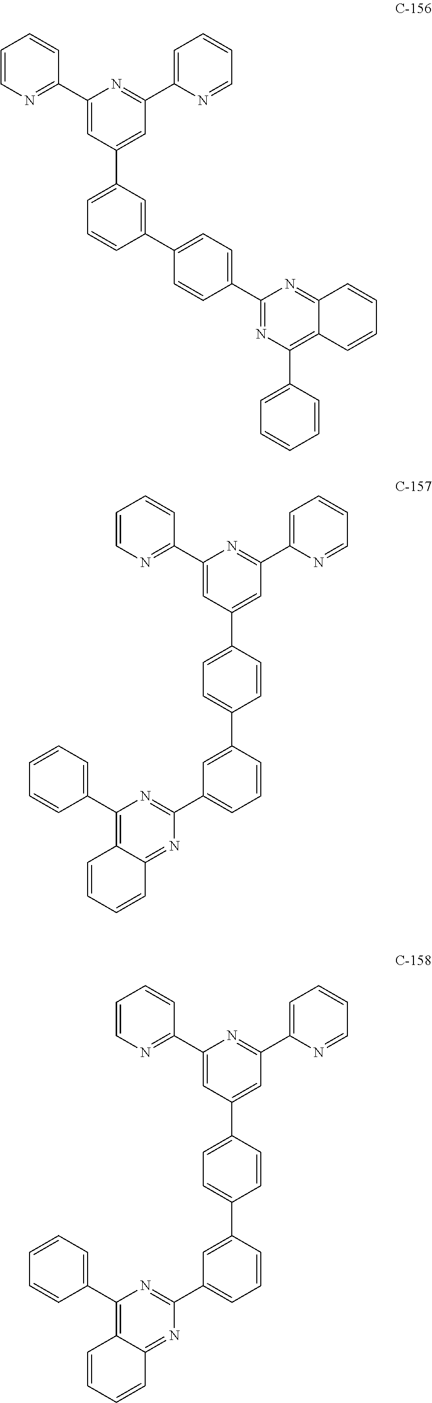 [SMILES: c1ccc(-c2nc(-c3ccc(-c4cccc(-c5cc(-c6ccccn6)nc(-c6ccccn6)c5)c4)cc3)nc3ccccc23)cc1 ; c1ccc(-c2nc(-c3cccc(-c4ccc(-c5cc(-c6ccccn6)nc(-c6ccccn6)c5)cc4)c3)nc3ccccc23)cc1 ; c1ccc(-c2nc(-c3cccc(-c4ccc(-c5cc(-c6ccccn6)nc(-c6ccccn6)c5)cc4)c3)nc3ccccc23)cc1]

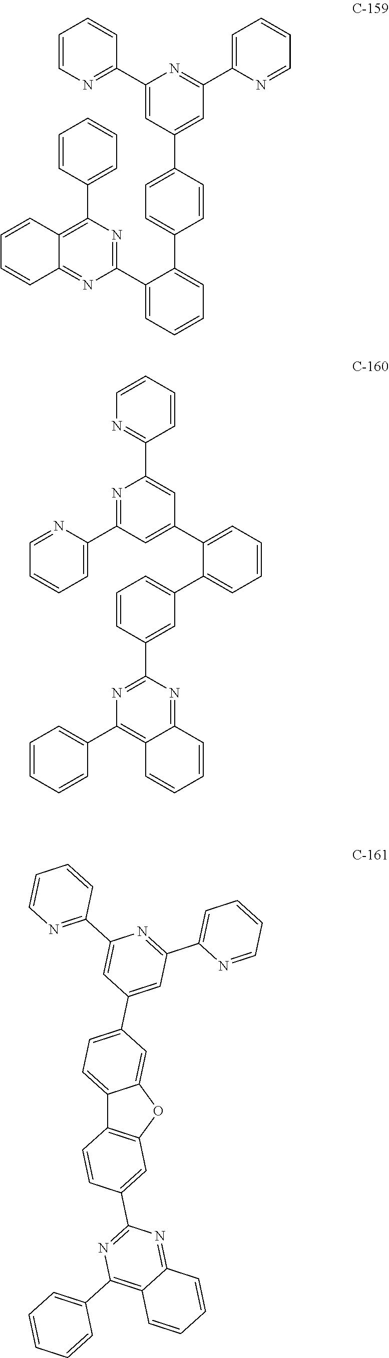 [SMILES: c1ccc(-c2nc(-c3ccccc3-c3ccc(-c4cc(-c5ccccn5)nc(-c5ccccn5)c4)cc3)nc3ccccc23)cc1 ; c1ccc(-c2nc(-c3cccc(-c4ccccc4-c4cc(-c5ccccn5)nc(-c5ccccn5)c4)c3)nc3ccccc23)cc1 ; c1ccc(-c2nc(-c3ccc4c(c3)oc3cc(-c5cc(-c6ccccn6)nc(-c6ccccn6)c5)ccc34)nc3ccccc23)cc1]

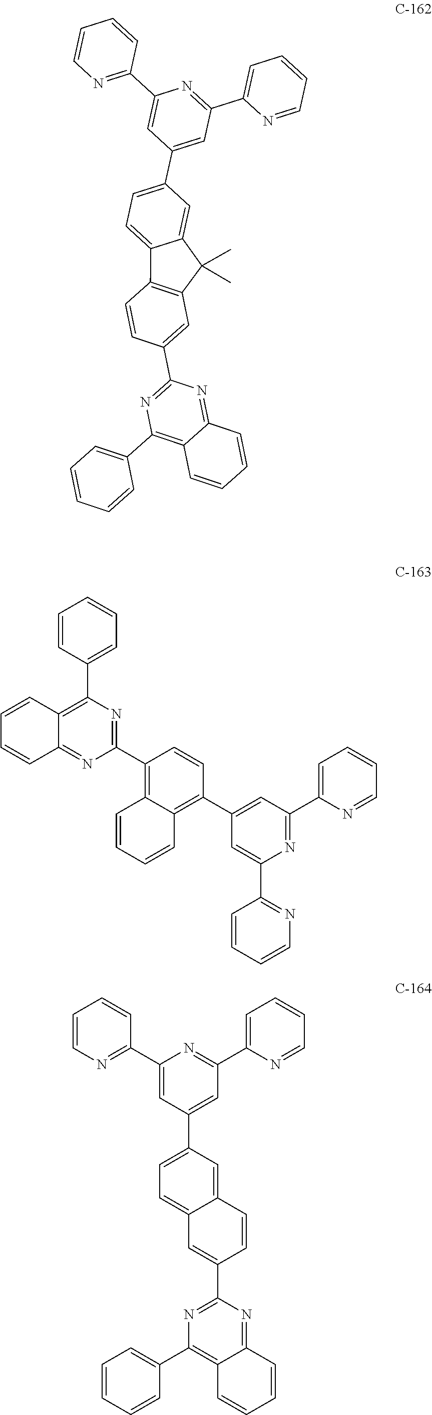

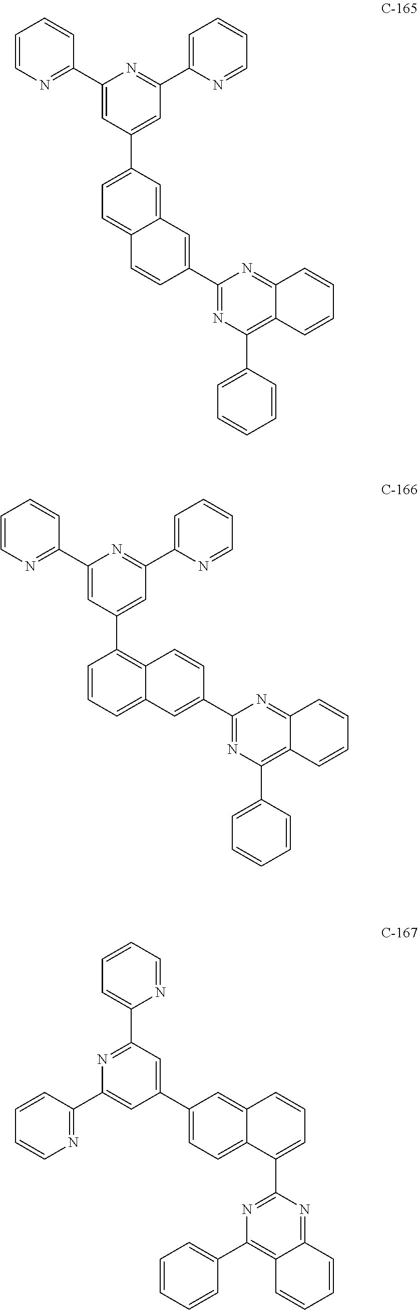 [SMILES: c1ccc(-c2nc(-c3ccc4ccc(-c5cc(-c6ccccn6)nc(-c6ccccn6)c5)cc4c3)nc3ccccc23)cc1 ; c1ccc(-c2nc(-c3ccc4c(-c5cc(-c6ccccn6)nc(-c6ccccn6)c5)cccc4c3)nc3ccccc23)cc1 ; c1ccc(-c2nc(-c3cccc4cc(-c5cc(-c6ccccn6)nc(-c6ccccn6)c5)ccc34)nc3ccccc23)cc1]

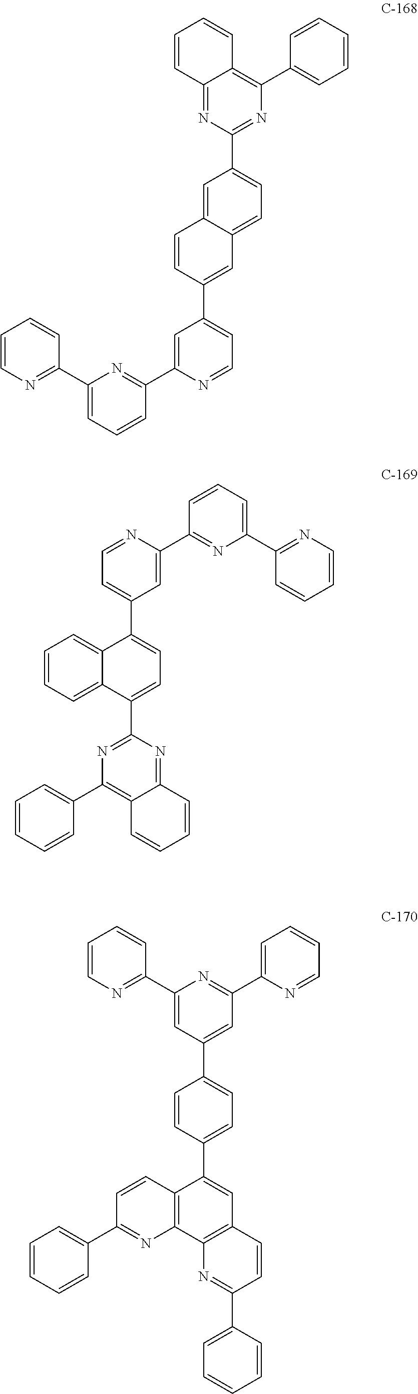 [SMILES: c1ccc(-c2nc(-c3ccc4cc(-c5ccnc(-c6cccc(-c7ccccn7)n6)c5)ccc4c3)nc3ccccc23)cc1 ; c1ccc(-c2nc(-c3ccc(-c4ccnc(-c5cccc(-c6ccccn6)n5)c4)c4ccccc34)nc3ccccc23)cc1 ; c1ccc(-c2ccc3cc(-c4ccc(-c5cc(-c6ccccn6)nc(-c6ccccn6)c5)cc4)c4ccc(-c5ccccc5)nc4c3n2)cc1]

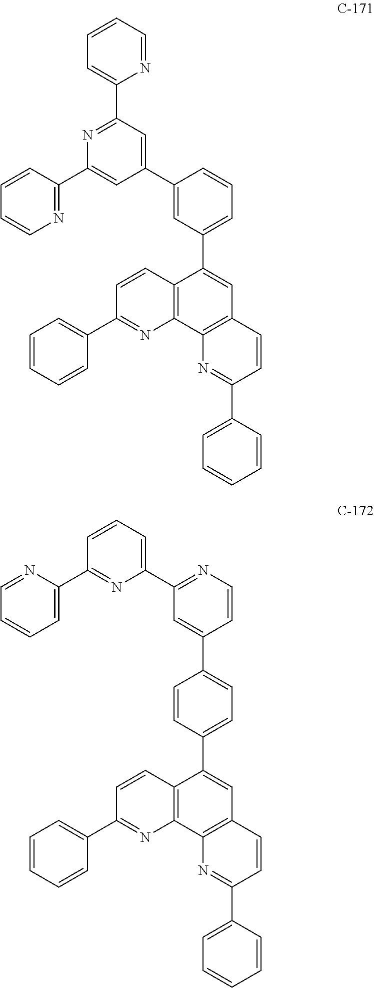 [SMILES: c1ccc(-c2ccc3cc(-c4cccc(-c5cc(-c6ccccn6)nc(-c6ccccn6)c5)c4)c4ccc(-c5ccccc5)nc4c3n2)cc1 ; c1ccc(-c2ccc3cc(-c4ccc(-c5ccnc(-c6cccc(-c7ccccn7)n6)c5)cc4)c4ccc(-c5ccccc5)nc4c3n2)cc1]

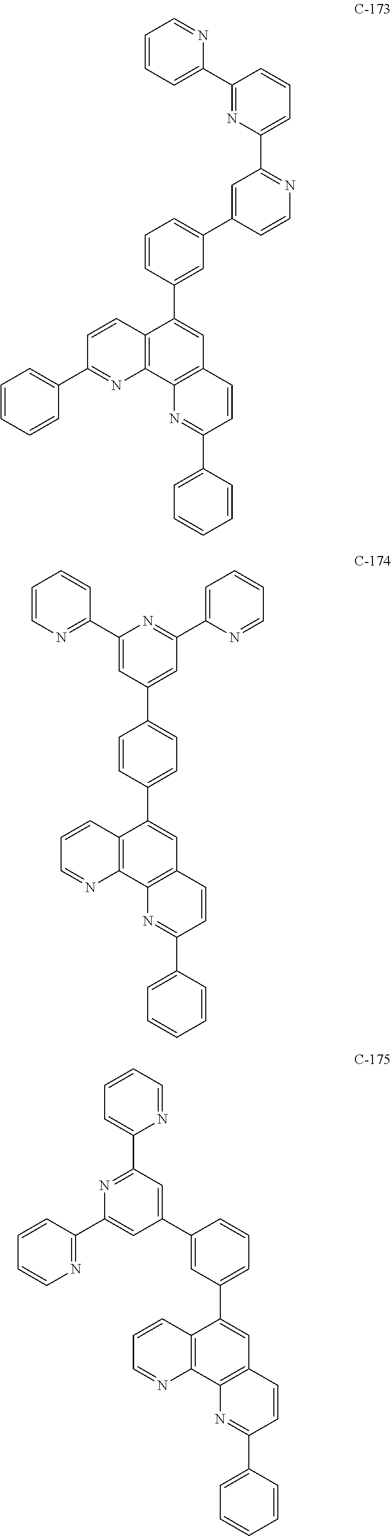 [SMILES: c1ccc(-c2ccc3cc(-c4cccc(-c5ccnc(-c6cccc(-c7ccccn7)n6)c5)c4)c4ccc(-c5ccccc5)nc4c3n2)cc1 ; c1ccc(-c2ccc3cc(-c4ccc(-c5cc(-c6ccccn6)nc(-c6ccccn6)c5)cc4)c4cccnc4c3n2)cc1 ; c1ccc(-c2ccc3cc(-c4cccc(-c5cc(-c6ccccn6)nc(-c6ccccn6)c5)c4)c4cccnc4c3n2)cc1]

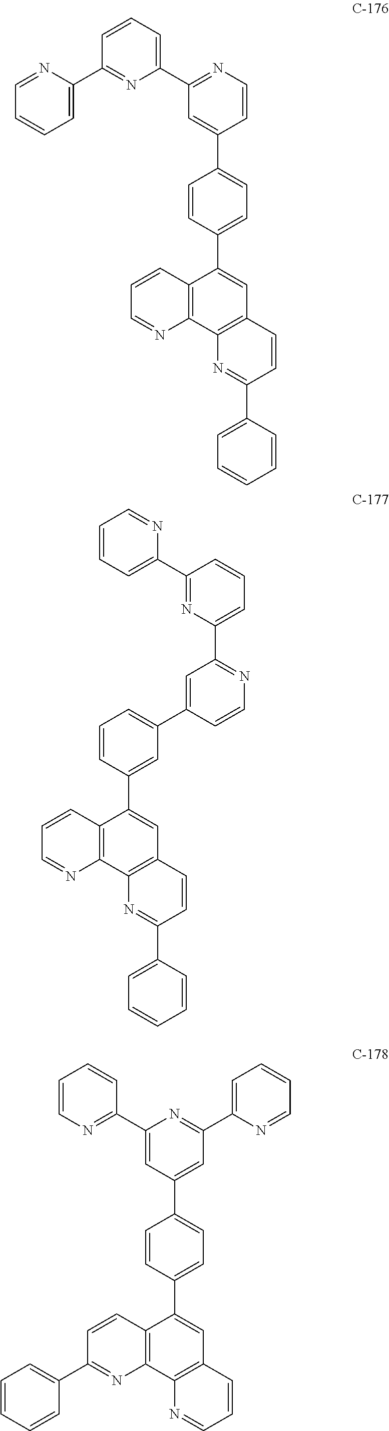 [SMILES: c1ccc(-c2ccc3cc(-c4ccc(-c5ccnc(-c6cccc(-c7ccccn7)n6)c5)cc4)c4cccnc4c3n2)cc1 ; c1ccc(-c2ccc3cc(-c4cccc(-c5ccnc(-c6cccc(-c7ccccn7)n6)c5)c4)c4cccnc4c3n2)cc1 ; c1ccc(-c2ccc3c(-c4ccc(-c5cc(-c6ccccn6)nc(-c6ccccn6)c5)cc4)cc4cccnc4c3n2)cc1]

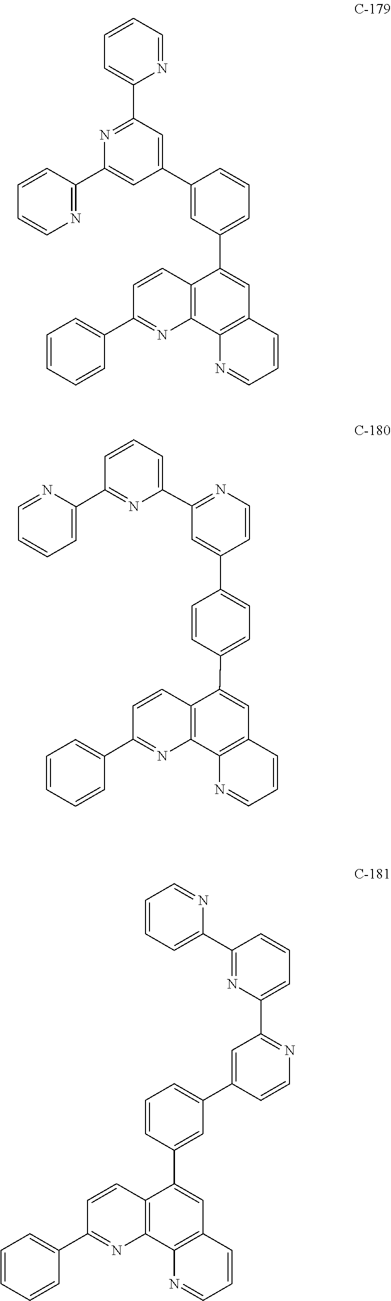 [SMILES: c1ccc(-c2ccc3c(-c4cccc(-c5cc(-c6ccccn6)nc(-c6ccccn6)c5)c4)cc4cccnc4c3n2)cc1 ; c1ccc(-c2ccc3c(-c4ccc(-c5ccnc(-c6cccc(-c7ccccn7)n6)c5)cc4)cc4cccnc4c3n2)cc1 ; c1ccc(-c2ccc3c(-c4cccc(-c5ccnc(-c6cccc(-c7ccccn7)n6)c5)c4)cc4cccnc4c3n2)cc1]

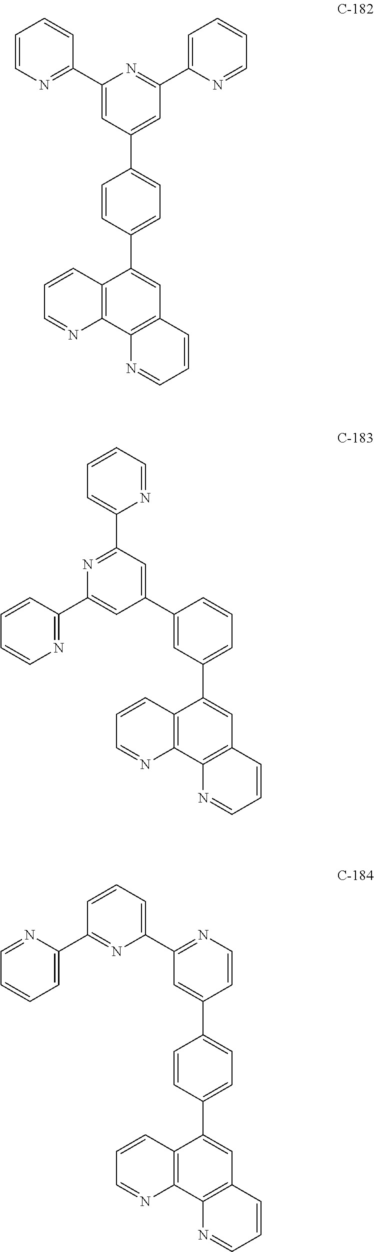 [SMILES: c1ccc(-c2cc(-c3ccc(-c4cc5cccnc5c5ncccc45)cc3)cc(-c3ccccn3)n2)nc1 ; c1ccc(-c2cc(-c3cccc(-c4cc5cccnc5c5ncccc45)c3)cc(-c3ccccn3)n2)nc1 ; c1ccc(-c2cccc(-c3cc(-c4ccc(-c5cc6cccnc6c6ncccc56)cc4)ccn3)n2)nc1]

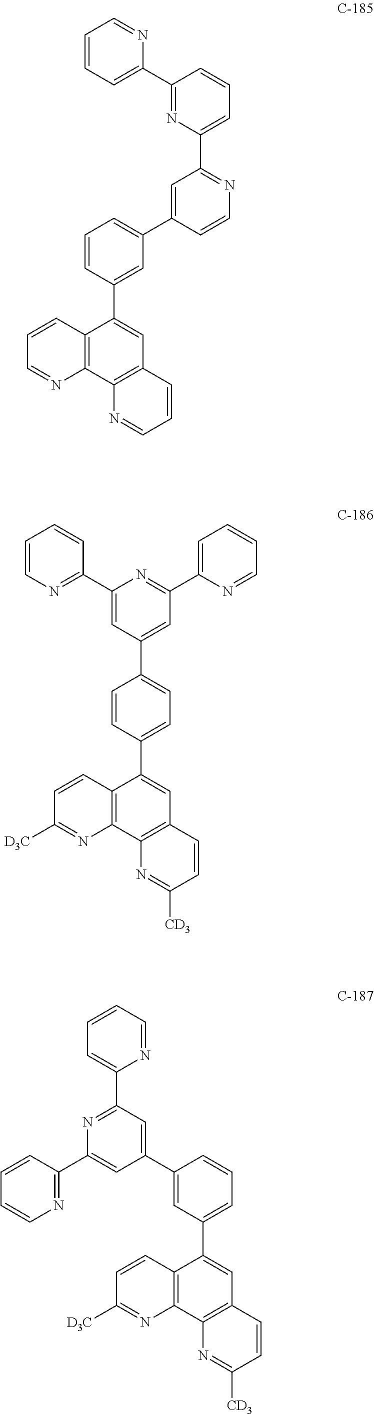 [SMILES: c1ccc(-c2cccc(-c3cc(-c4cccc(-c5cc6cccnc6c6ncccc56)c4)ccn3)n2)nc1 ; [2H]C([2H])([2H])c1ccc2cc(-c3ccc(-c4cc(-c5ccccn5)nc(-c5ccccn5)c4)cc3)c3ccc(C([2H])([2H])[2H])nc3c2n1 ; [2H]C([2H])([2H])c1ccc2cc(-c3cccc(-c4cc(-c5ccccn5)nc(-c5ccccn5)c4)c3)c3ccc(C([2H])([2H])[2H])nc3c2n1]

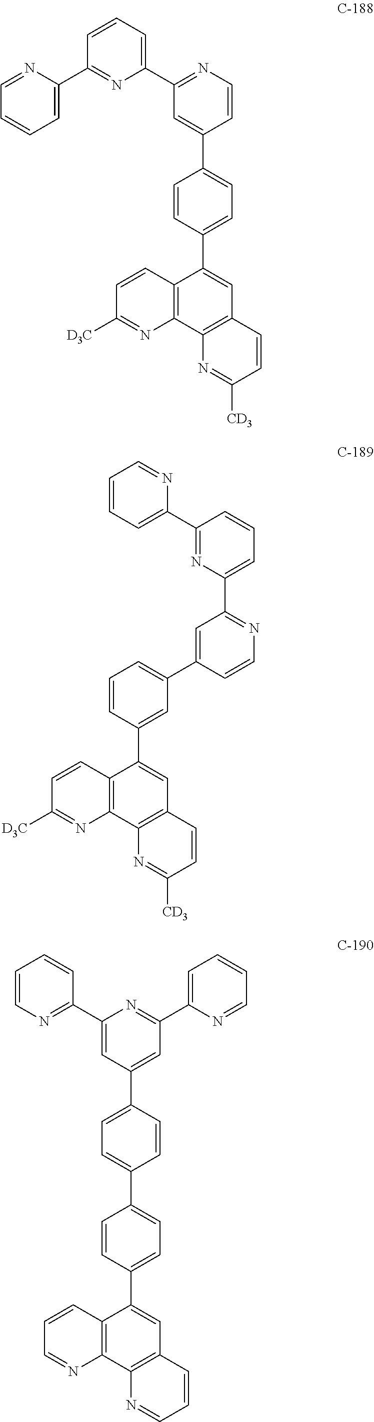 [SMILES: [2H]C([2H])([2H])c1ccc2cc(-c3ccc(-c4ccnc(-c5cccc(-c6ccccn6)n5)c4)cc3)c3ccc(C([2H])([2H])[2H])nc3c2n1 ; [2H]C([2H])([2H])c1ccc2cc(-c3cccc(-c4ccnc(-c5cccc(-c6ccccn6)n5)c4)c3)c3ccc(C([2H])([2H])[2H])nc3c2n1 ; c1ccc(-c2cc(-c3ccc(-c4ccc(-c5cc6cccnc6c6ncccc56)cc4)cc3)cc(-c3ccccn3)n2)nc1]

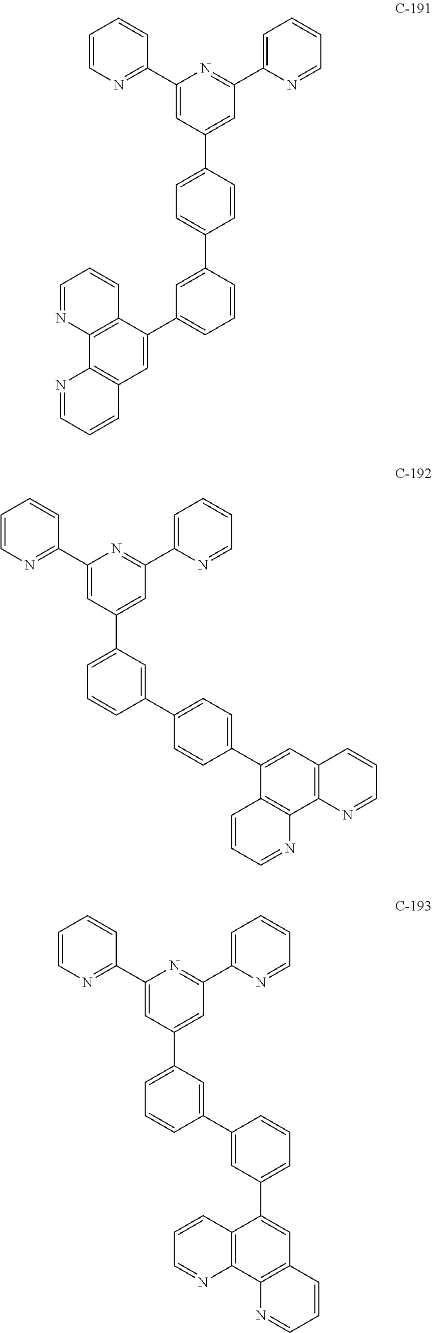 [SMILES: c1ccc(-c2cc(-c3ccc(-c4cccc(-c5cc6cccnc6c6ncccc56)c4)cc3)cc(-c3ccccn3)n2)nc1 ; c1ccc(-c2cc(-c3cccc(-c4ccc(-c5cc6cccnc6c6ncccc56)cc4)c3)cc(-c3ccccn3)n2)nc1 ; c1ccc(-c2cc(-c3cccc(-c4cccc(-c5cc6cccnc6c6ncccc56)c4)c3)cc(-c3ccccn3)n2)nc1]

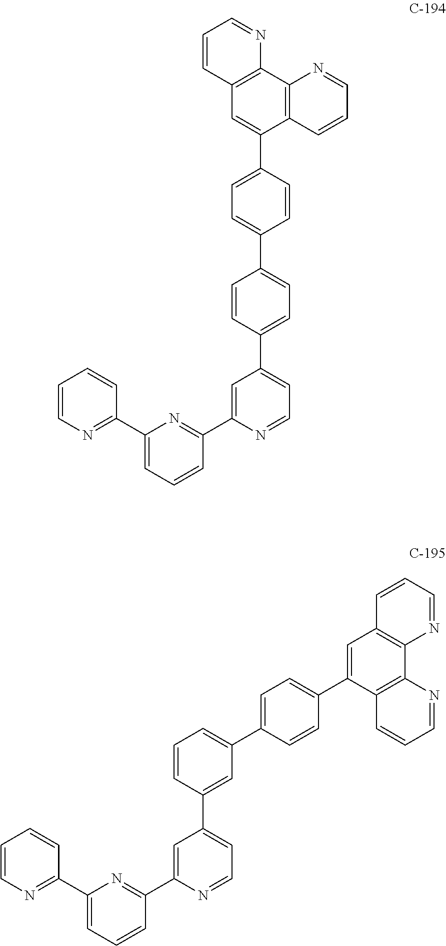 [SMILES: c1ccc(-c2cccc(-c3cc(-c4ccc(-c5ccc(-c6cc7cccnc7c7ncccc67)cc5)cc4)ccn3)n2)nc1 ; c1ccc(-c2cccc(-c3cc(-c4cccc(-c5ccc(-c6cc7cccnc7c7ncccc67)cc5)c4)ccn3)n2)nc1]

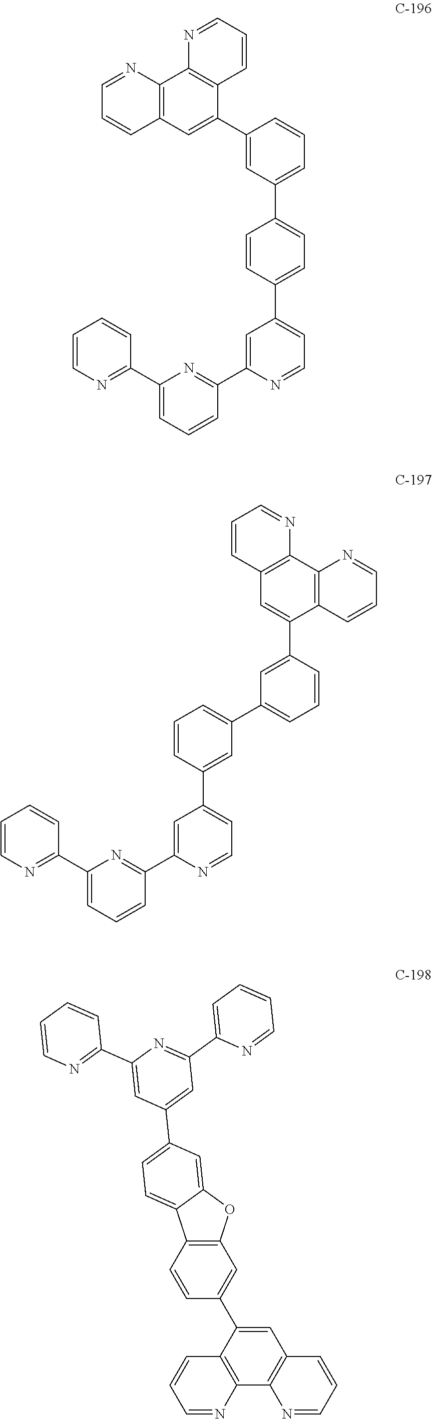 [SMILES: c1ccc(-c2cccc(-c3cc(-c4ccc(-c5cccc(-c6cc7cccnc7c7ncccc67)c5)cc4)ccn3)n2)nc1 ; c1ccc(-c2cccc(-c3cc(-c4cccc(-c5cccc(-c6cc7cccnc7c7ncccc67)c5)c4)ccn3)n2)nc1 ; c1ccc(-c2cc(-c3ccc4c(c3)oc3cc(-c5cc6cccnc6c6ncccc56)ccc34)cc(-c3ccccn3)n2)nc1]

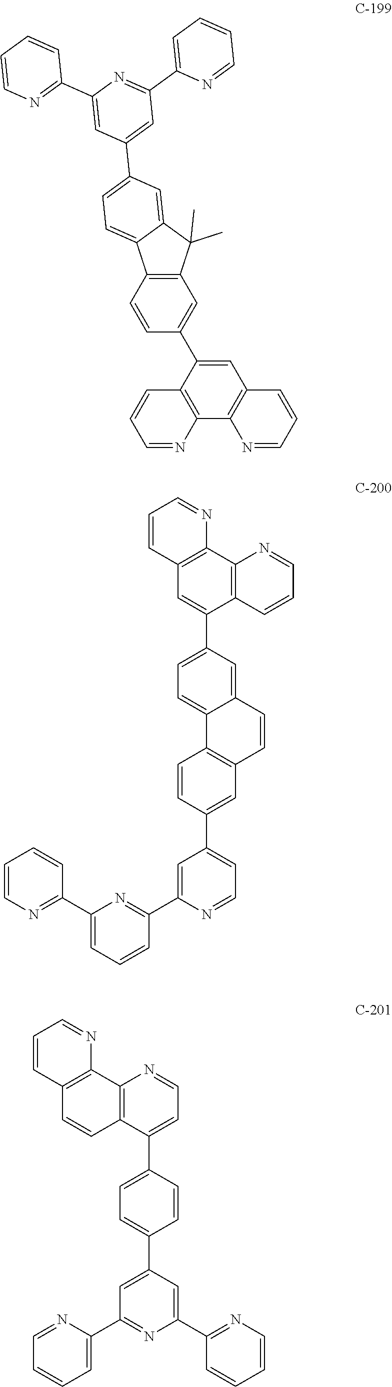 [SMILES: CC1(C)c2cc(-c3cc(-c4ccccn4)nc(-c4ccccn4)c3)ccc2-c2ccc(-c3cc4cccnc4c4ncccc34)cc21 ; c1ccc(-c2cccc(-c3cc(-c4ccc5c(ccc6cc(-c7cc8cccnc8c8ncccc78)ccc65)c4)ccn3)n2)nc1 ; c1ccc(-c2cc(-c3ccc(-c4ccnc5c4ccc4cccnc45)cc3)cc(-c3ccccn3)n2)nc1]

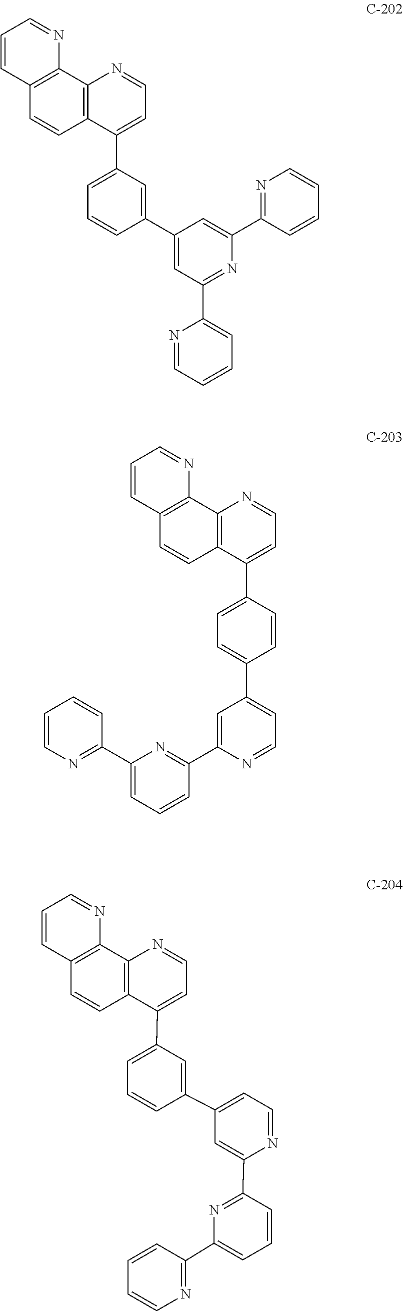 [SMILES: c1ccc(-c2cc(-c3cccc(-c4ccnc5c4ccc4cccnc45)c3)cc(-c3ccccn3)n2)nc1 ; c1ccc(-c2cccc(-c3cc(-c4ccc(-c5ccnc6c5ccc5cccnc56)cc4)ccn3)n2)nc1 ; c1ccc(-c2cccc(-c3cc(-c4cccc(-c5ccnc6c5ccc5cccnc56)c4)ccn3)n2)nc1]

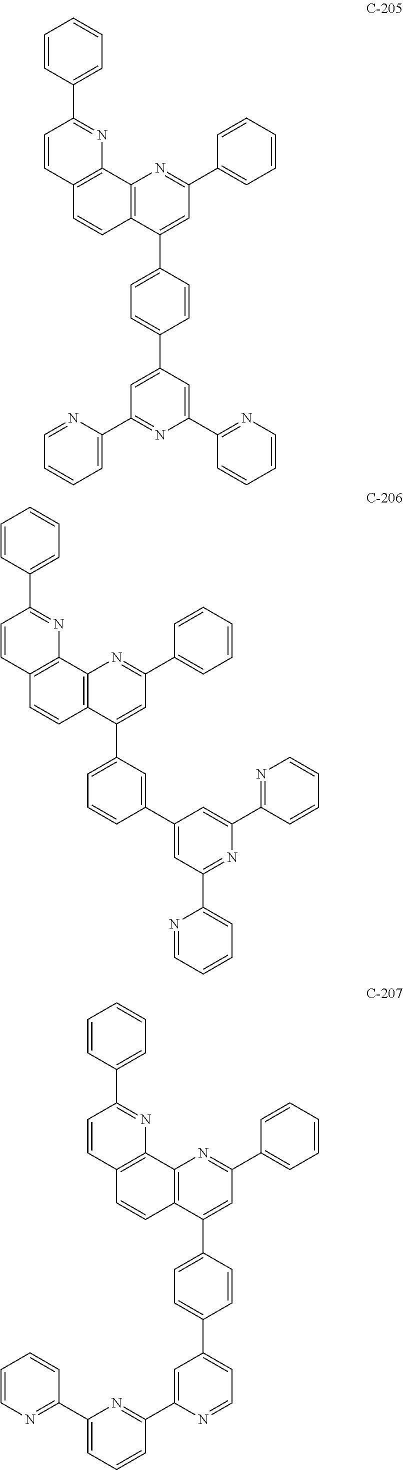

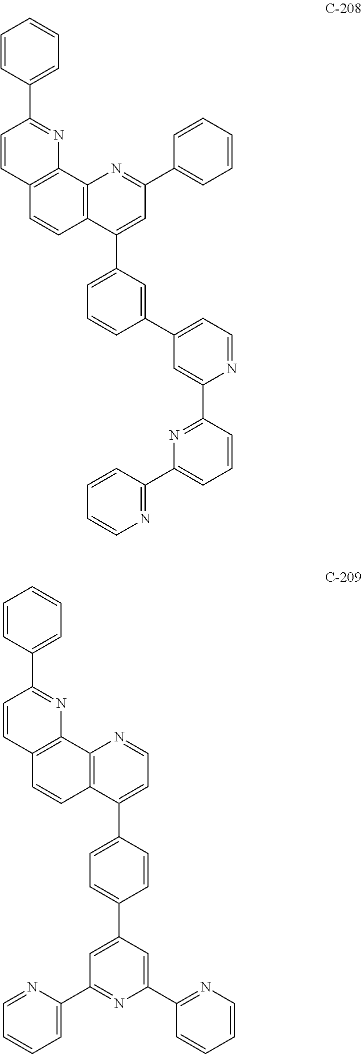 [SMILES: c1ccc(-c2ccc3ccc4c(-c5cccc(-c6ccnc(-c7cccc(-c8ccccn8)n7)c6)c5)cc(-c5ccccc5)nc4c3n2)cc1 ; c1ccc(-c2ccc3ccc4c(-c5ccc(-c6cc(-c7ccccn7)nc(-c7ccccn7)c6)cc5)ccnc4c3n2)cc1]

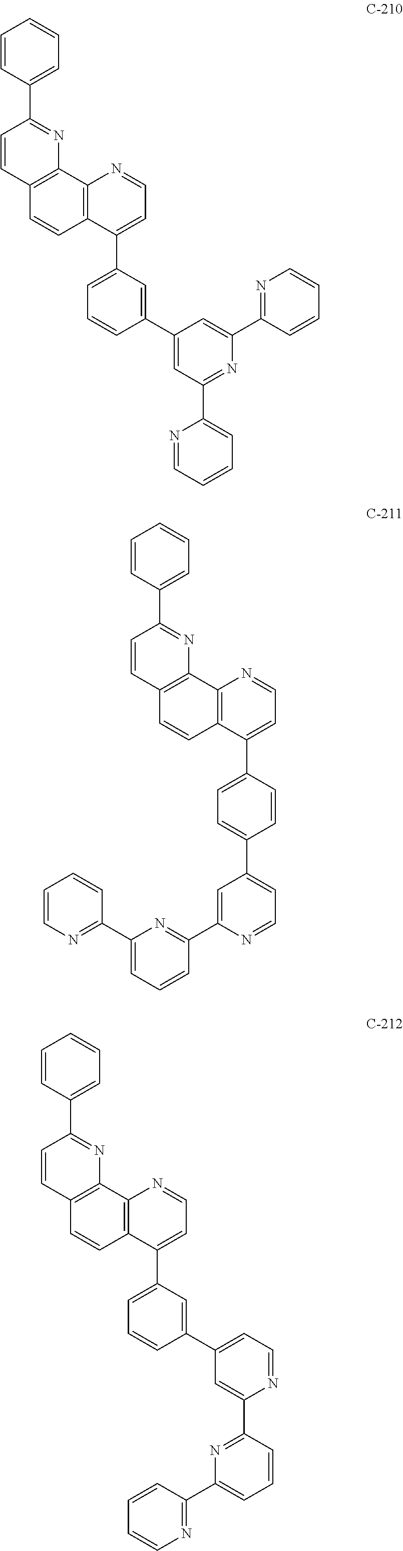 [SMILES: c1ccc(-c2ccc3ccc4c(-c5cccc(-c6cc(-c7ccccn7)nc(-c7ccccn7)c6)c5)ccnc4c3n2)cc1 ; c1ccc(-c2ccc3ccc4c(-c5ccc(-c6ccnc(-c7cccc(-c8ccccn8)n7)c6)cc5)ccnc4c3n2)cc1 ; c1ccc(-c2ccc3ccc4c(-c5cccc(-c6ccnc(-c7cccc(-c8ccccn8)n7)c6)c5)ccnc4c3n2)cc1]

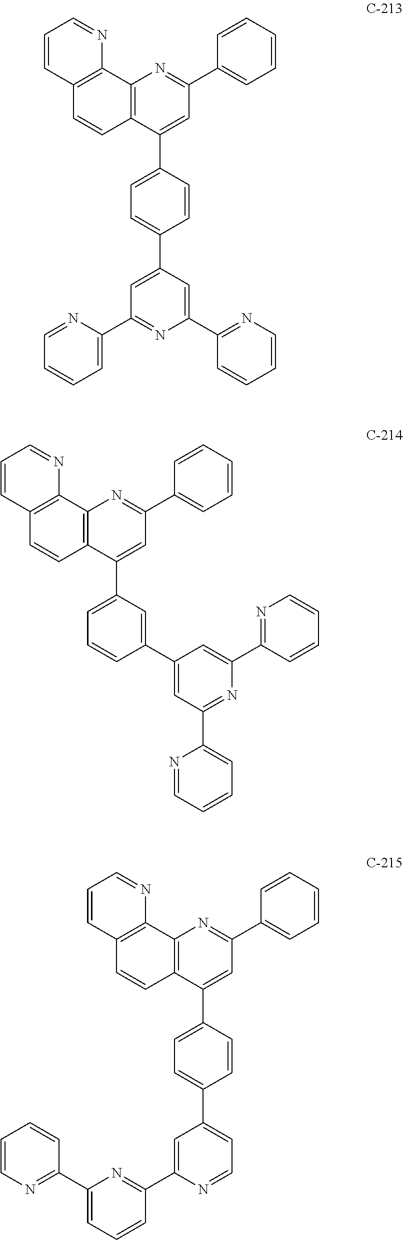 [SMILES: c1ccc(-c2cc(-c3ccc(-c4cc(-c5ccccn5)nc(-c5ccccn5)c4)cc3)c3ccc4cccnc4c3n2)cc1 ; c1ccc(-c2cc(-c3cccc(-c4cc(-c5ccccn5)nc(-c5ccccn5)c4)c3)c3ccc4cccnc4c3n2)cc1 ; c1ccc(-c2cc(-c3ccc(-c4ccnc(-c5cccc(-c6ccccn6)n5)c4)cc3)c3ccc4cccnc4c3n2)cc1]

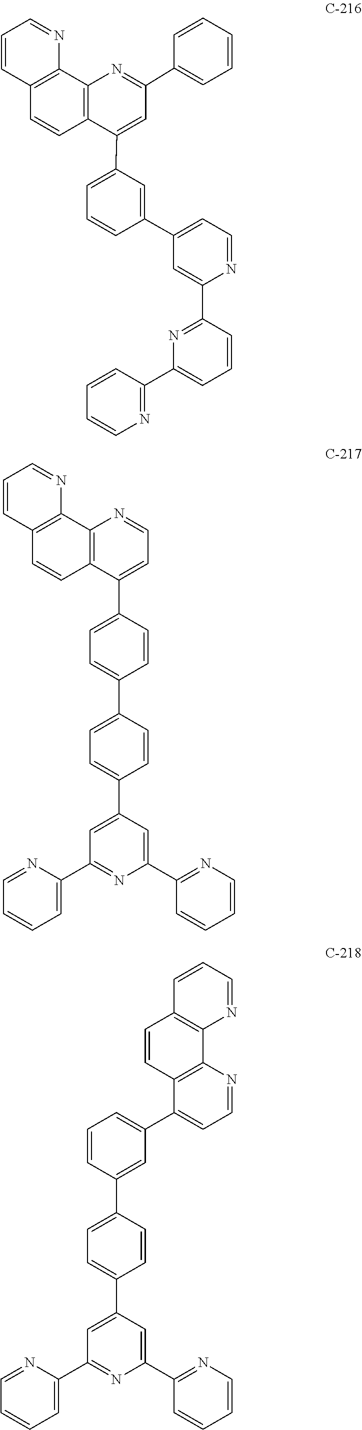

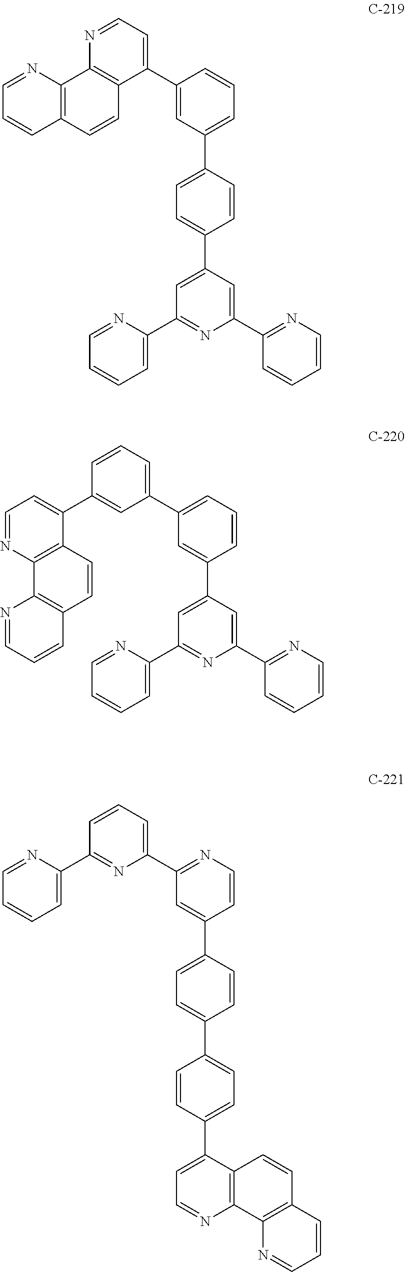 [SMILES: c1ccc(-c2cc(-c3ccc(-c4cccc(-c5ccnc6c5ccc5cccnc56)c4)cc3)cc(-c3ccccn3)n2)nc1 ; c1ccc(-c2cc(-c3cccc(-c4cccc(-c5ccnc6c5ccc5cccnc56)c4)c3)cc(-c3ccccn3)n2)nc1 ; c1ccc(-c2cccc(-c3cc(-c4ccc(-c5ccc(-c6ccnc7c6ccc6cccnc67)cc5)cc4)ccn3)n2)nc1]

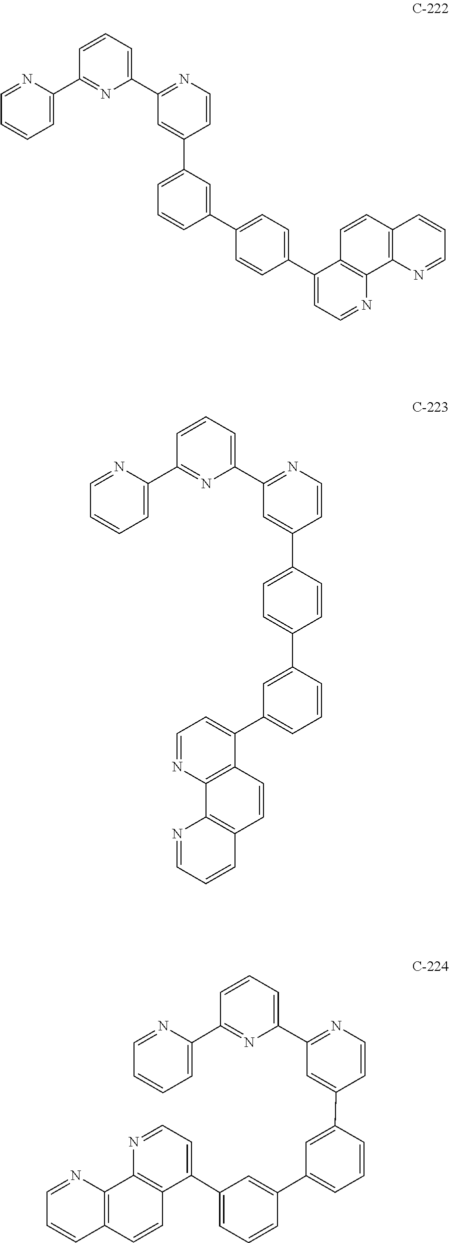 [SMILES: c1ccc(-c2cccc(-c3cc(-c4cccc(-c5ccc(-c6ccnc7c6ccc6cccnc67)cc5)c4)ccn3)n2)nc1 ; c1ccc(-c2cccc(-c3cc(-c4ccc(-c5cccc(-c6ccnc7c6ccc6cccnc67)c5)cc4)ccn3)n2)nc1 ; c1ccc(-c2cccc(-c3cc(-c4cccc(-c5cccc(-c6ccnc7c6ccc6cccnc67)c5)c4)ccn3)n2)nc1]

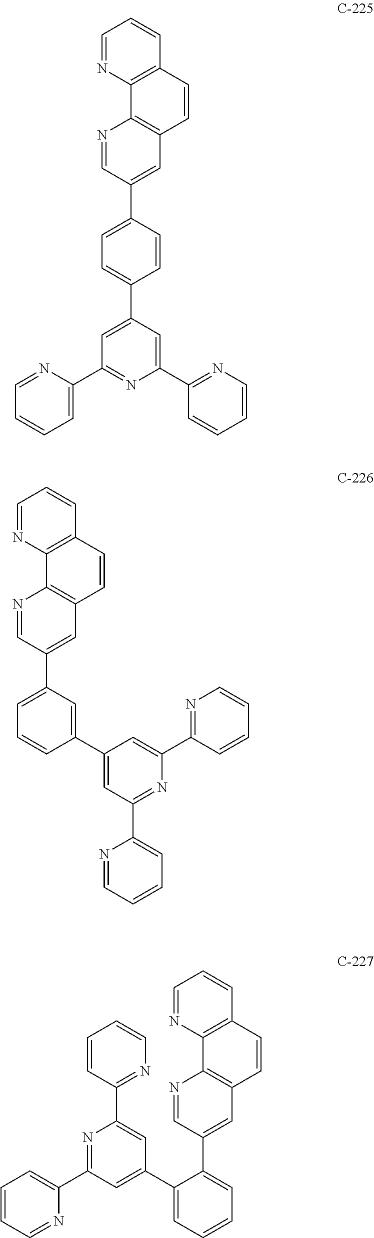 [SMILES: c1ccc(-c2cc(-c3ccc(-c4cnc5c(ccc6cccnc65)c4)cc3)cc(-c3ccccn3)n2)nc1 ; c1ccc(-c2cc(-c3cccc(-c4cnc5c(ccc6cccnc65)c4)c3)cc(-c3ccccn3)n2)nc1 ; c1ccc(-c2cc(-c3ccccc3-c3cnc4c(ccc5cccnc54)c3)cc(-c3ccccn3)n2)nc1]

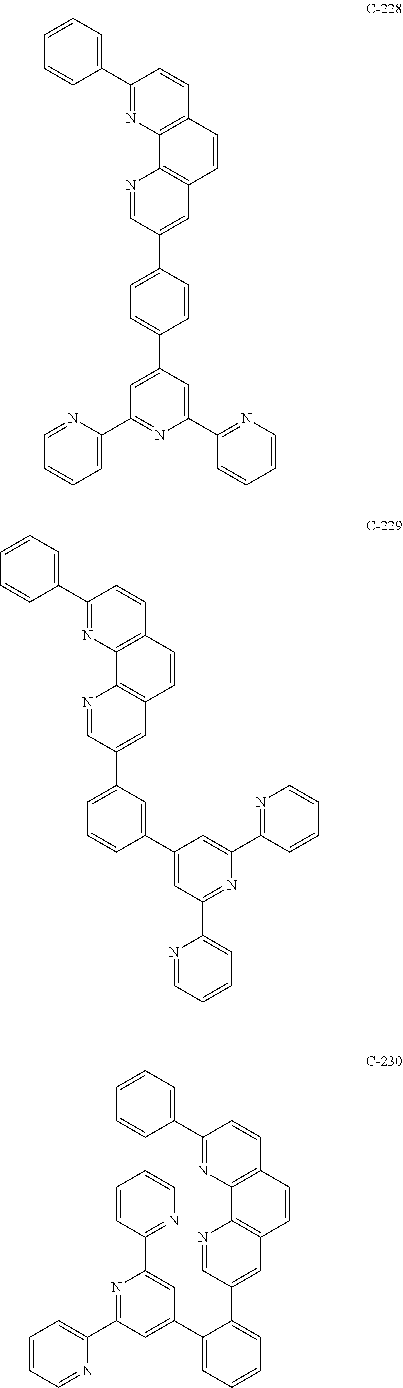 [SMILES: c1ccc(-c2ccc3ccc4cc(-c5ccc(-c6cc(-c7ccccn7)nc(-c7ccccn7)c6)cc5)cnc4c3n2)cc1 ; c1ccc(-c2ccc3ccc4cc(-c5cccc(-c6cc(-c7ccccn7)nc(-c7ccccn7)c6)c5)cnc4c3n2)cc1 ; c1ccc(-c2ccc3ccc4cc(-c5ccccc5-c5cc(-c6ccccn6)nc(-c6ccccn6)c5)cnc4c3n2)cc1]

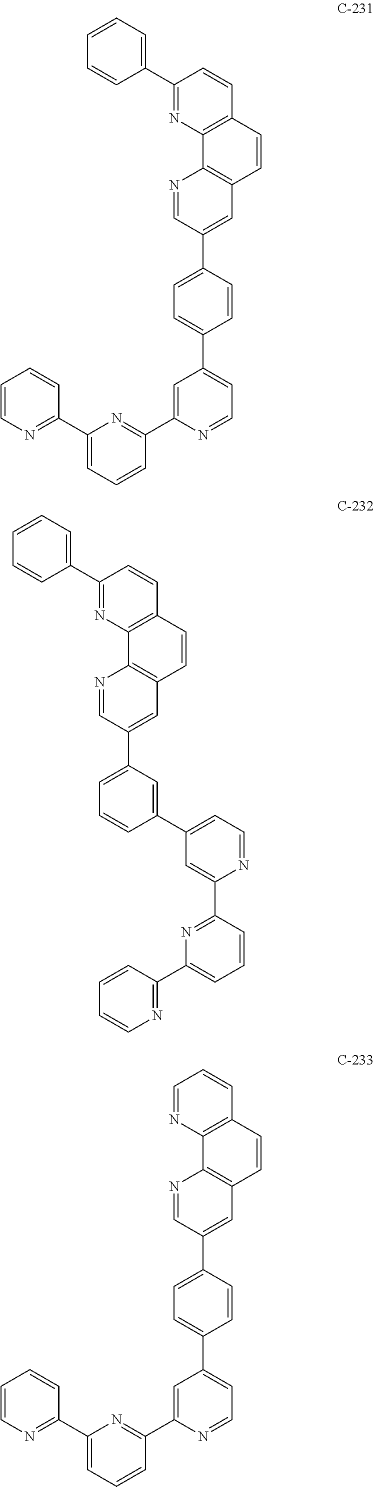 [SMILES: c1ccc(-c2ccc3ccc4cc(-c5ccc(-c6ccnc(-c7cccc(-c8ccccn8)n7)c6)cc5)cnc4c3n2)cc1 ; c1ccc(-c2ccc3ccc4cc(-c5cccc(-c6ccnc(-c7cccc(-c8ccccn8)n7)c6)c5)cnc4c3n2)cc1 ; c1ccc(-c2cccc(-c3cc(-c4ccc(-c5cnc6c(ccc7cccnc76)c5)cc4)ccn3)n2)nc1]

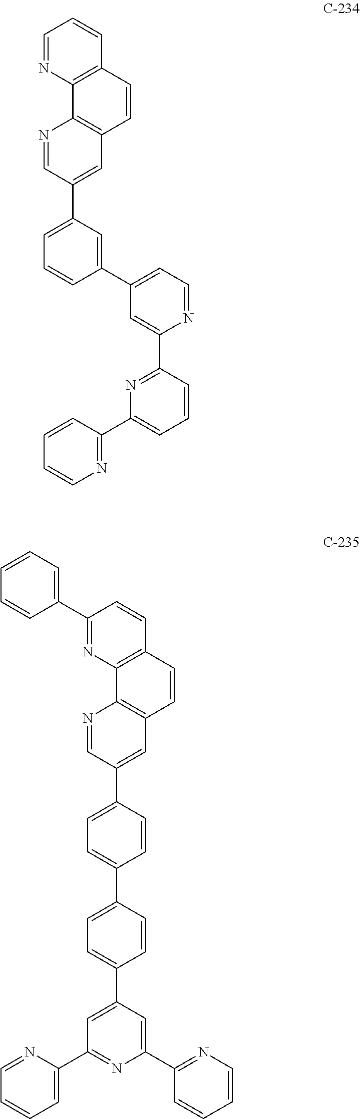 [SMILES: c1ccc(-c2cccc(-c3cc(-c4cccc(-c5cnc6c(ccc7cccnc76)c5)c4)ccn3)n2)nc1 ; c1ccc(-c2ccc3ccc4cc(-c5ccc(-c6ccc(-c7cc(-c8ccccn8)nc(-c8ccccn8)c7)cc6)cc5)cnc4c3n2)cc1]

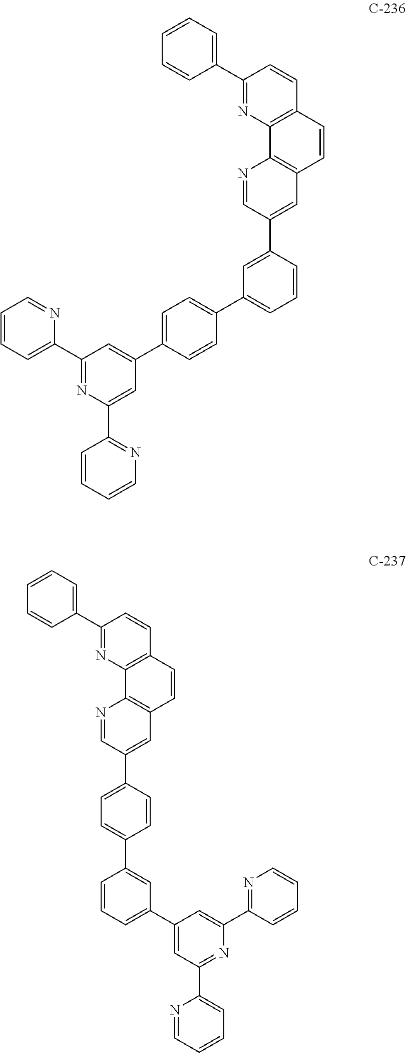 [SMILES: c1ccc(-c2ccc3ccc4cc(-c5cccc(-c6ccc(-c7cc(-c8ccccn8)nc(-c8ccccn8)c7)cc6)c5)cnc4c3n2)cc1 ; c1ccc(-c2ccc3ccc4cc(-c5ccc(-c6cccc(-c7cc(-c8ccccn8)nc(-c8ccccn8)c7)c6)cc5)cnc4c3n2)cc1]

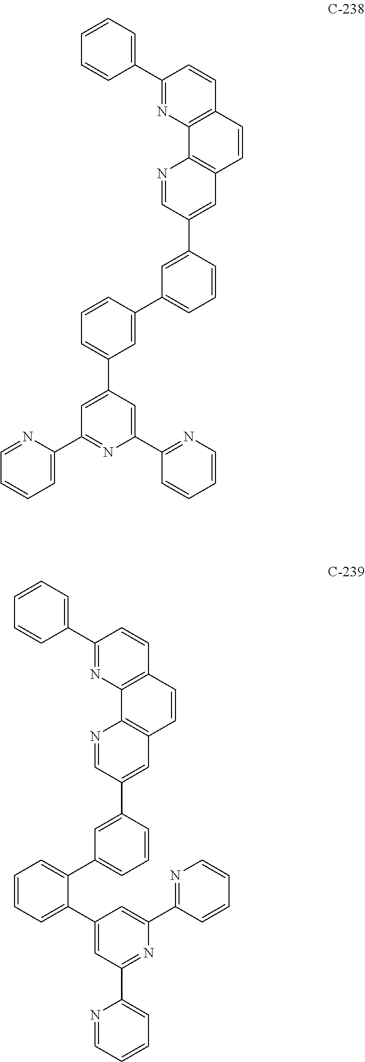 [SMILES: c1ccc(-c2ccc3ccc4cc(-c5cccc(-c6cccc(-c7cc(-c8ccccn8)nc(-c8ccccn8)c7)c6)c5)cnc4c3n2)cc1 ; c1ccc(-c2ccc3ccc4cc(-c5cccc(-c6ccccc6-c6cc(-c7ccccn7)nc(-c7ccccn7)c6)c5)cnc4c3n2)cc1]

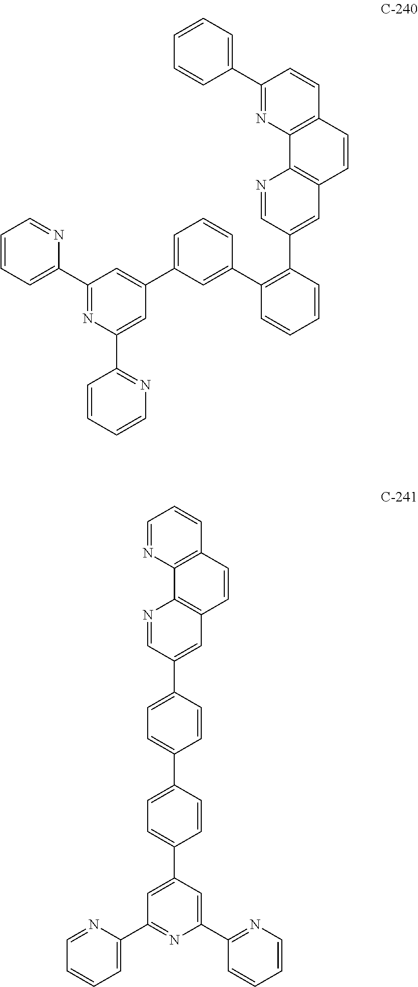 [SMILES: c1ccc(-c2ccc3ccc4cc(-c5ccccc5-c5cccc(-c6cc(-c7ccccn7)nc(-c7ccccn7)c6)c5)cnc4c3n2)cc1 ; c1ccc(-c2cc(-c3ccc(-c4ccc(-c5cnc6c(ccc7cccnc76)c5)cc4)cc3)cc(-c3ccccn3)n2)nc1]

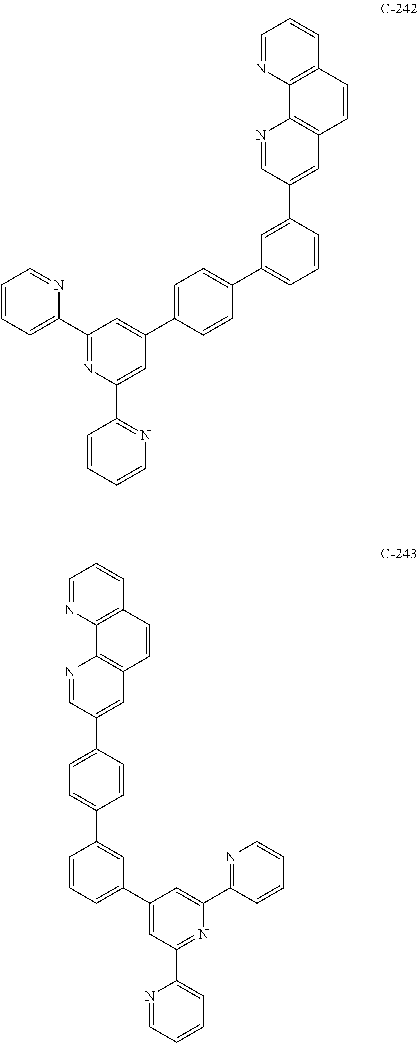 [SMILES: c1ccc(-c2cc(-c3ccc(-c4cccc(-c5cnc6c(ccc7cccnc76)c5)c4)cc3)cc(-c3ccccn3)n2)nc1 ; c1ccc(-c2cc(-c3cccc(-c4ccc(-c5cnc6c(ccc7cccnc76)c5)cc4)c3)cc(-c3ccccn3)n2)nc1]

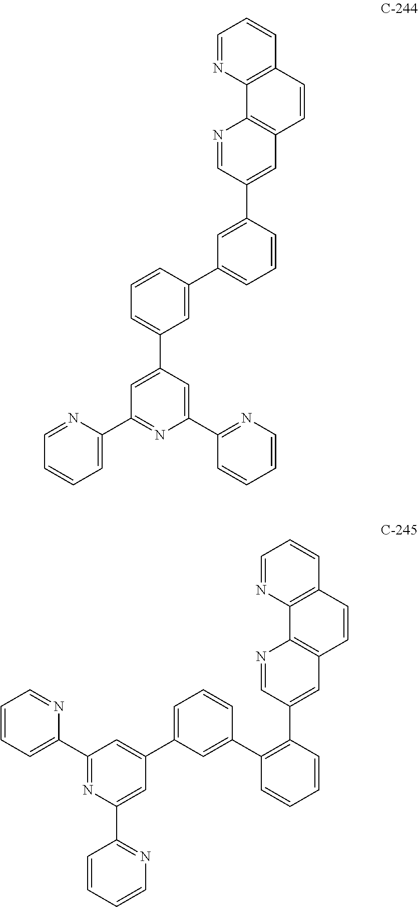 [SMILES: c1ccc(-c2cc(-c3cccc(-c4cccc(-c5cnc6c(ccc7cccnc76)c5)c4)c3)cc(-c3ccccn3)n2)nc1 ; c1ccc(-c2cc(-c3cccc(-c4ccccc4-c4cnc5c(ccc6cccnc65)c4)c3)cc(-c3ccccn3)n2)nc1]

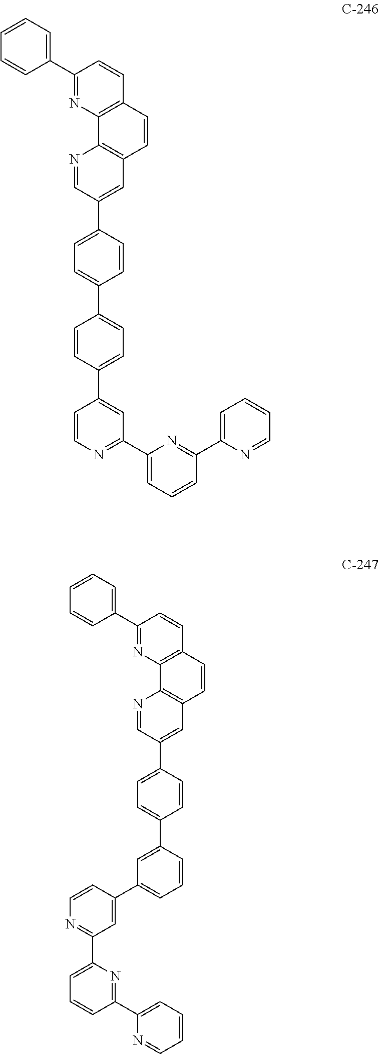 [SMILES: c1ccc(-c2ccc3ccc4cc(-c5ccc(-c6ccc(-c7ccnc(-c8cccc(-c9ccccn9)n8)c7)cc6)cc5)cnc4c3n2)cc1 ; c1ccc(-c2ccc3ccc4cc(-c5ccc(-c6cccc(-c7ccnc(-c8cccc(-c9ccccn9)n8)c7)c6)cc5)cnc4c3n2)cc1]

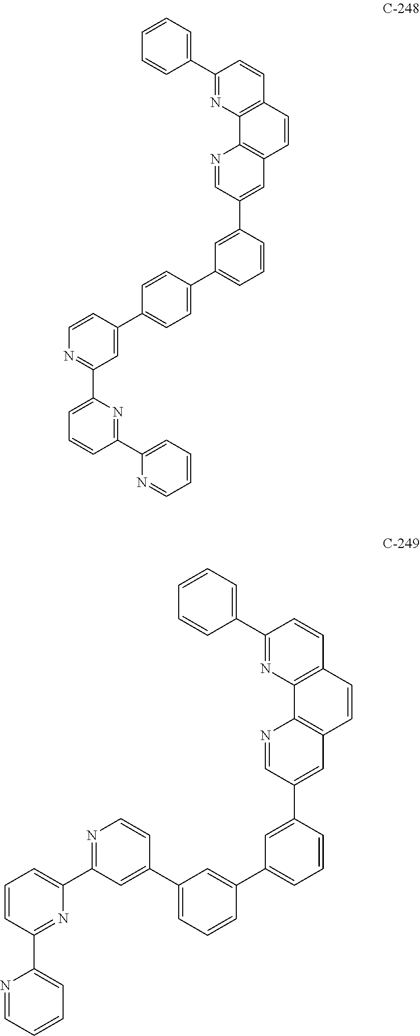 [SMILES: c1ccc(-c2ccc3ccc4cc(-c5cccc(-c6ccc(-c7ccnc(-c8cccc(-c9ccccn9)n8)c7)cc6)c5)cnc4c3n2)cc1 ; c1ccc(-c2ccc3ccc4cc(-c5cccc(-c6cccc(-c7ccnc(-c8cccc(-c9ccccn9)n8)c7)c6)c5)cnc4c3n2)cc1]

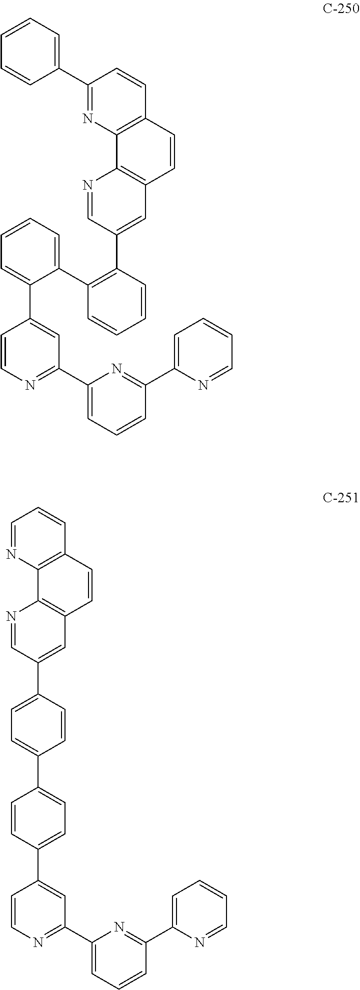 [SMILES: c1ccc(-c2ccc3ccc4cc(-c5ccccc5-c5ccccc5-c5ccnc(-c6cccc(-c7ccccn7)n6)c5)cnc4c3n2)cc1 ; c1ccc(-c2cccc(-c3cc(-c4ccc(-c5ccc(-c6cnc7c(ccc8cccnc87)c6)cc5)cc4)ccn3)n2)nc1]

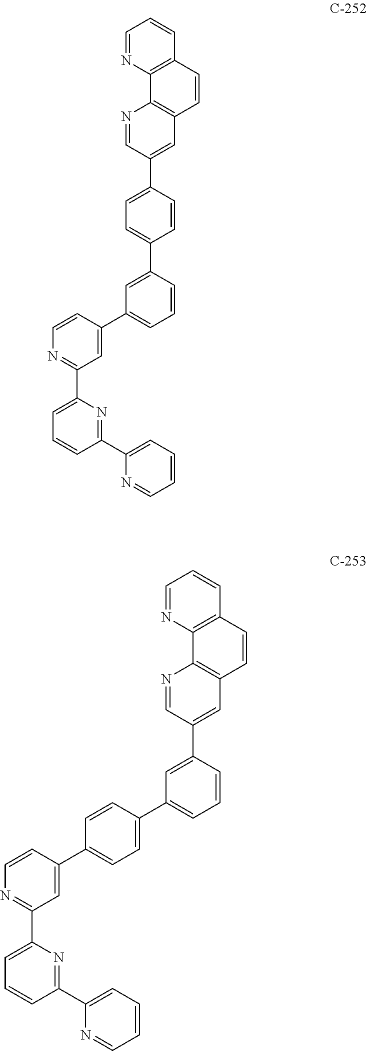 [SMILES: c1ccc(-c2cccc(-c3cc(-c4cccc(-c5ccc(-c6cnc7c(ccc8cccnc87)c6)cc5)c4)ccn3)n2)nc1 ; c1ccc(-c2cccc(-c3cc(-c4ccc(-c5cccc(-c6cnc7c(ccc8cccnc87)c6)c5)cc4)ccn3)n2)nc1]

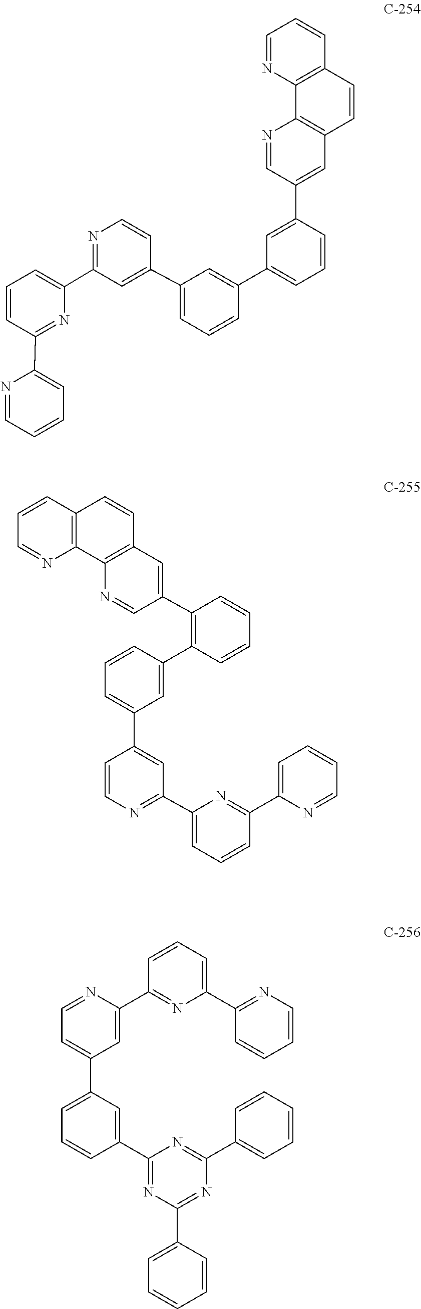 [SMILES: c1ccc(-c2cccc(-c3cc(-c4cccc(-c5cccc(-c6cnc7c(ccc8cccnc87)c6)c5)c4)ccn3)n2)nc1 ; c1ccc(-c2cccc(-c3cc(-c4cccc(-c5ccccc5-c5cnc6c(ccc7cccnc76)c5)c4)ccn3)n2)nc1 ; c1ccc(-c2nc(-c3ccccc3)nc(-c3cccc(-c4ccnc(-c5cccc(-c6ccccn6)n5)c4)c3)n2)cc1]

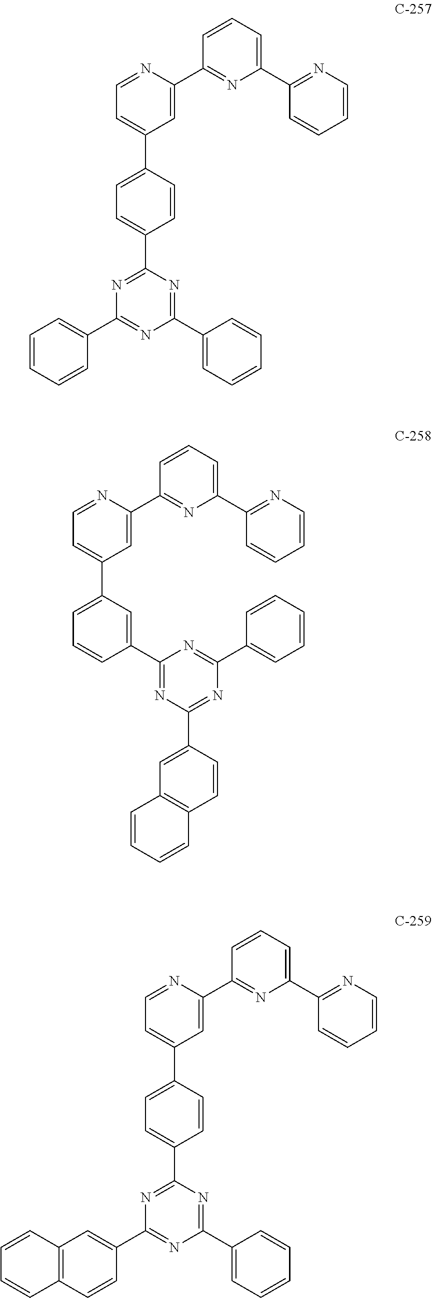 [SMILES: c1ccc(-c2nc(-c3ccccc3)nc(-c3ccc(-c4ccnc(-c5cccc(-c6ccccn6)n5)c4)cc3)n2)cc1 ; c1ccc(-c2nc(-c3cccc(-c4ccnc(-c5cccc(-c6ccccn6)n5)c4)c3)nc(-c3ccc4ccccc4c3)n2)cc1 ; c1ccc(-c2nc(-c3ccc(-c4ccnc(-c5cccc(-c6ccccn6)n5)c4)cc3)nc(-c3ccc4ccccc4c3)n2)cc1]

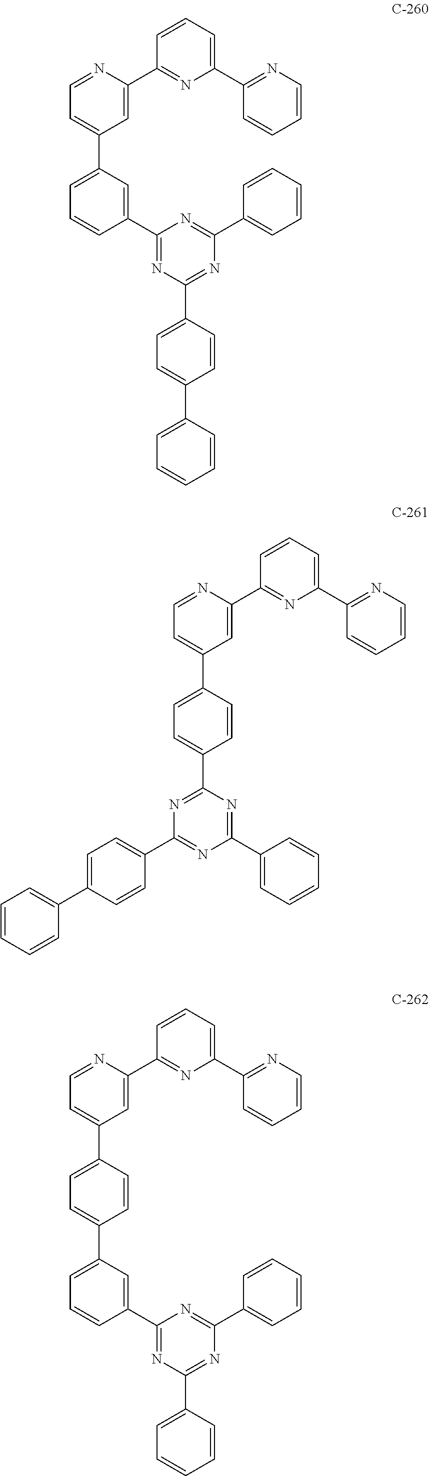 [SMILES: c1ccc(-c2ccc(-c3nc(-c4ccccc4)nc(-c4cccc(-c5ccnc(-c6cccc(-c7ccccn7)n6)c5)c4)n3)cc2)cc1 ; c1ccc(-c2ccc(-c3nc(-c4ccccc4)nc(-c4ccc(-c5ccnc(-c6cccc(-c7ccccn7)n6)c5)cc4)n3)cc2)cc1 ; c1ccc(-c2nc(-c3ccccc3)nc(-c3cccc(-c4ccc(-c5ccnc(-c6cccc(-c7ccccn7)n6)c5)cc4)c3)n2)cc1]

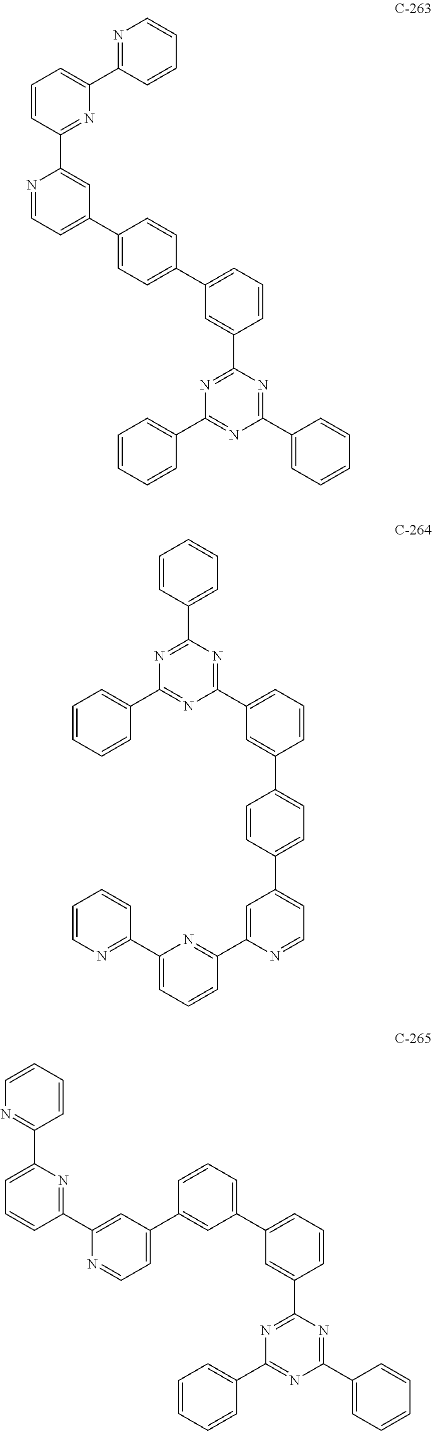 [SMILES: c1ccc(-c2nc(-c3ccccc3)nc(-c3cccc(-c4ccc(-c5ccnc(-c6cccc(-c7ccccn7)n6)c5)cc4)c3)n2)cc1 ; c1ccc(-c2nc(-c3ccccc3)nc(-c3cccc(-c4ccc(-c5ccnc(-c6cccc(-c7ccccn7)n6)c5)cc4)c3)n2)cc1 ; c1ccc(-c2nc(-c3ccccc3)nc(-c3cccc(-c4cccc(-c5ccnc(-c6cccc(-c7ccccn7)n6)c5)c4)c3)n2)cc1]

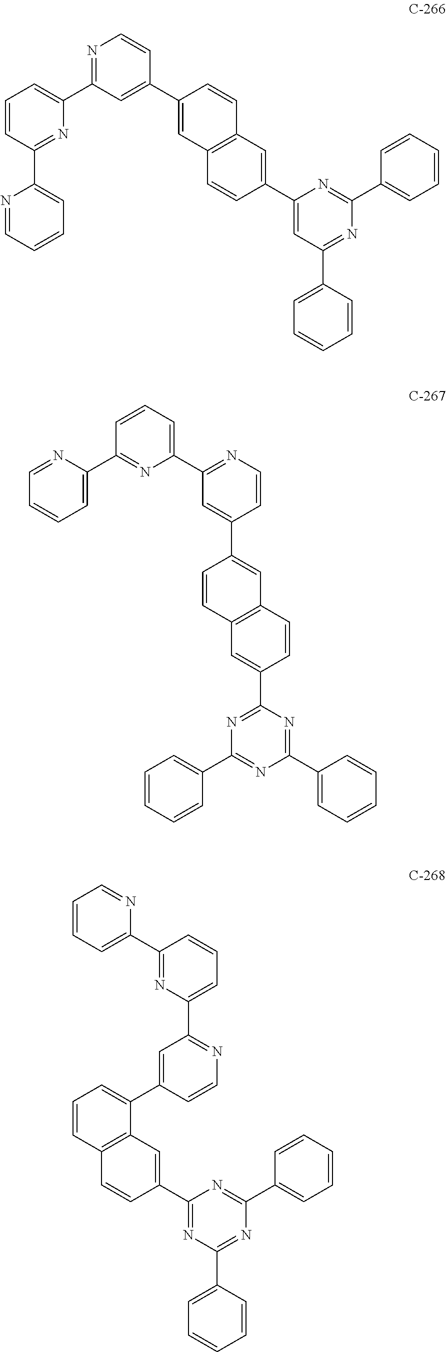

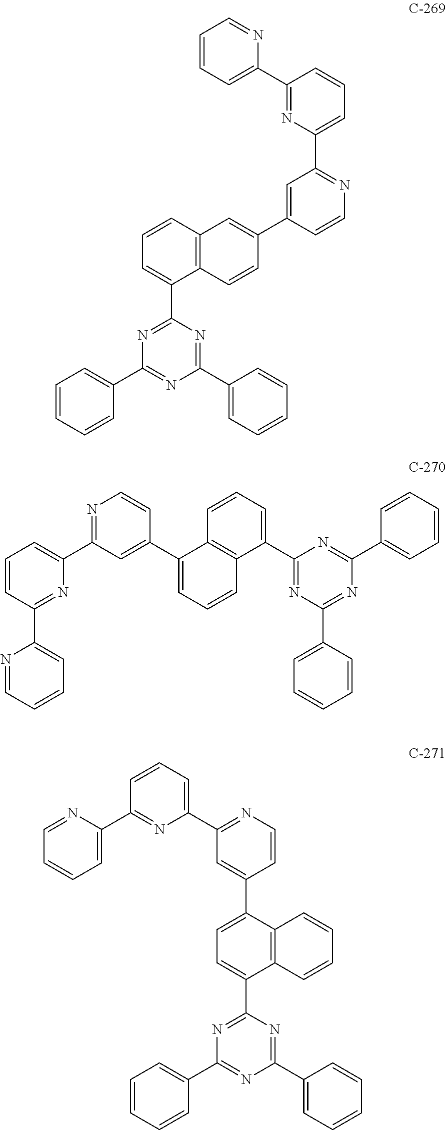 [SMILES: c1ccc(-c2nc(-c3ccccc3)nc(-c3cccc4cc(-c5ccnc(-c6cccc(-c7ccccn7)n6)c5)ccc34)n2)cc1 ; c1ccc(-c2nc(-c3ccccc3)nc(-c3cccc4c(-c5ccnc(-c6cccc(-c7ccccn7)n6)c5)cccc34)n2)cc1 ; c1ccc(-c2nc(-c3ccccc3)nc(-c3ccc(-c4ccnc(-c5cccc(-c6ccccn6)n5)c4)c4ccccc34)n2)cc1]

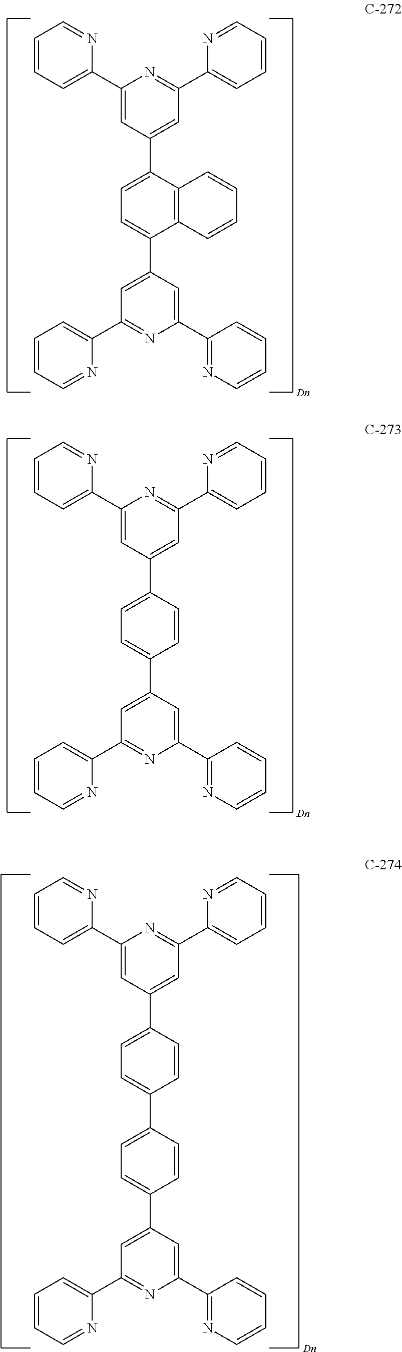

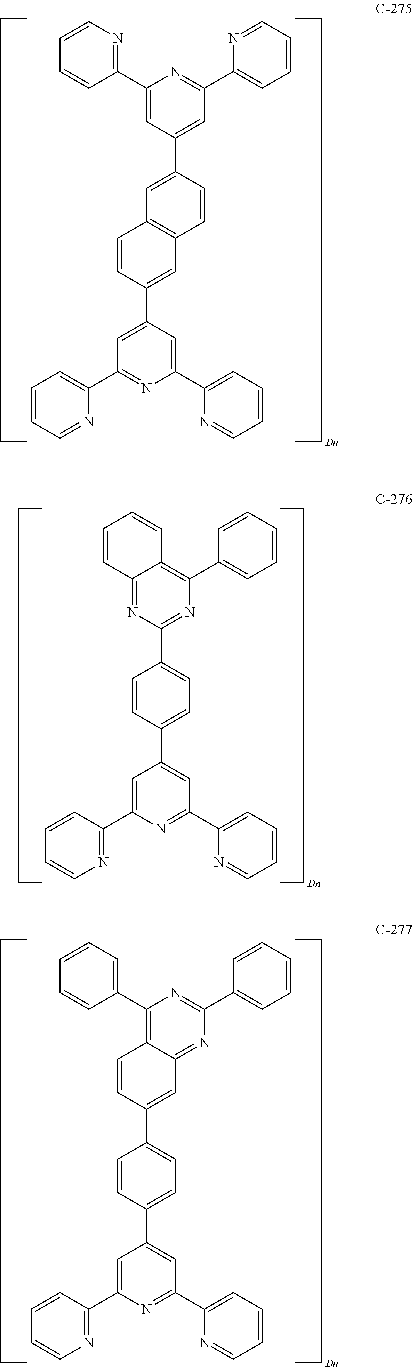

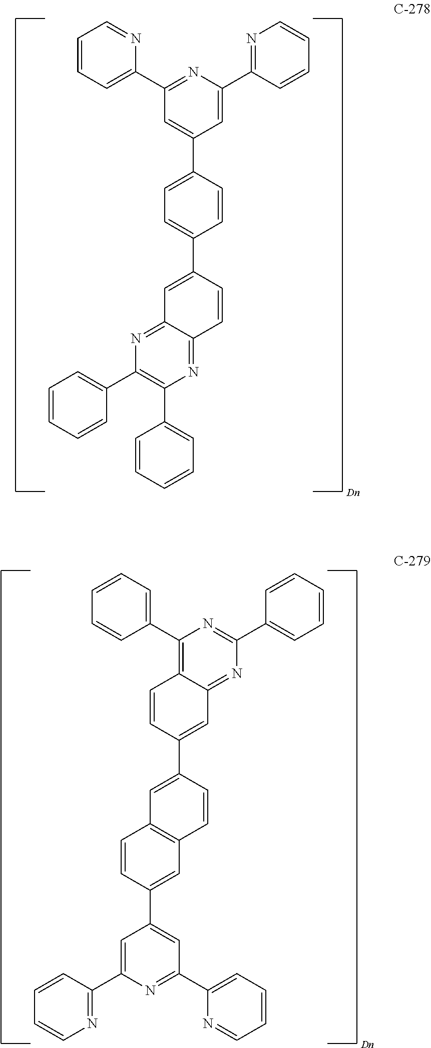

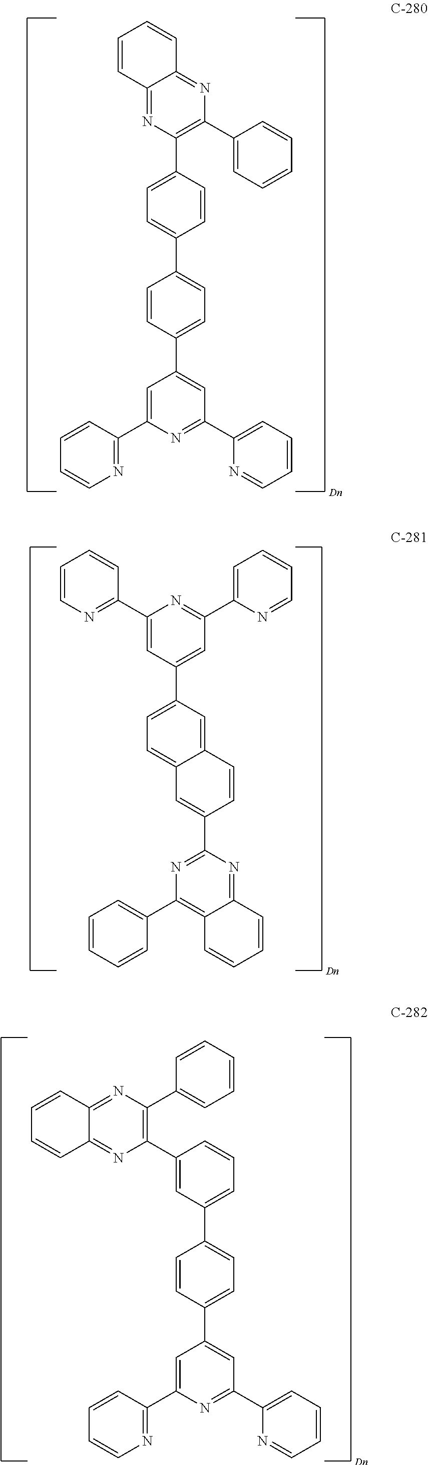

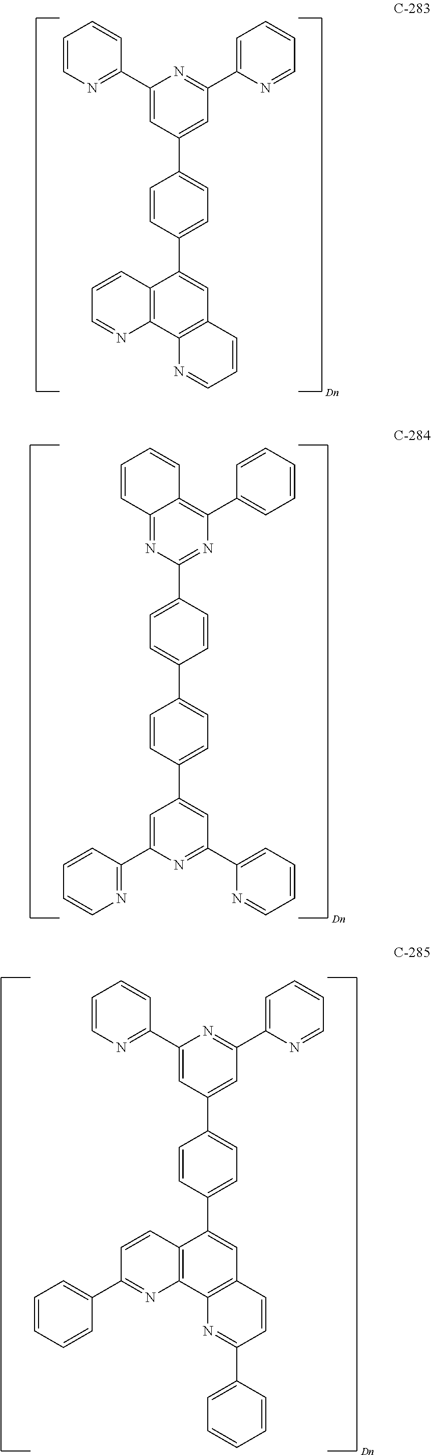

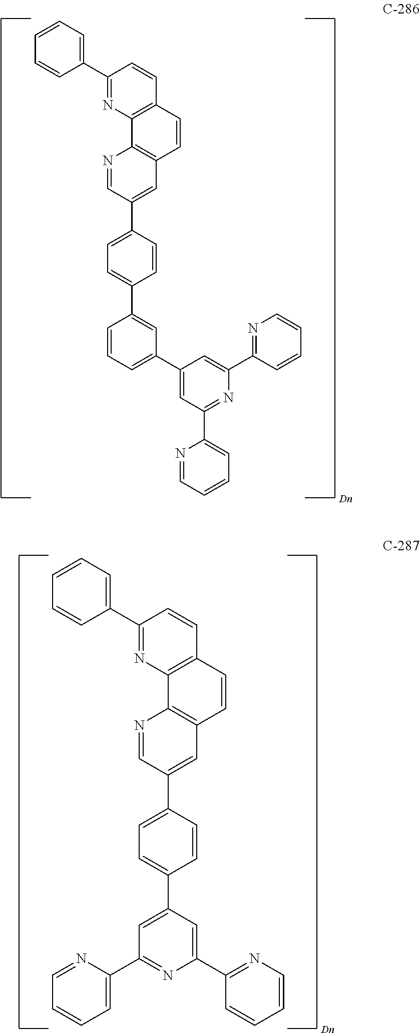

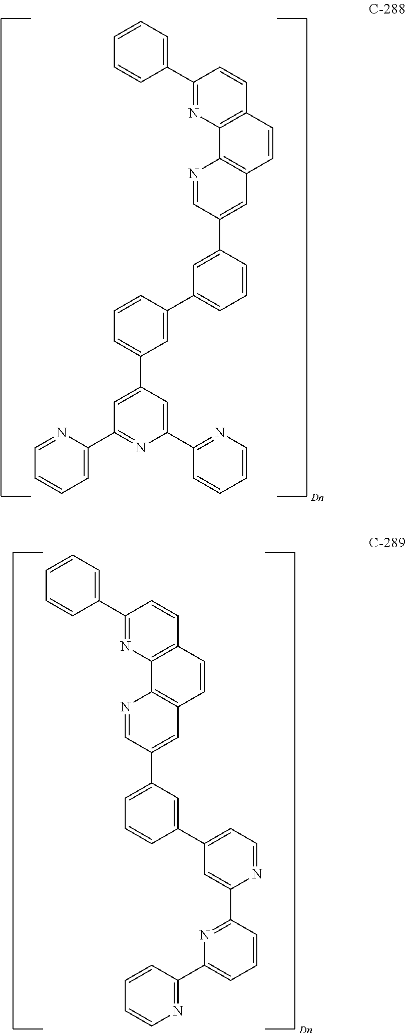

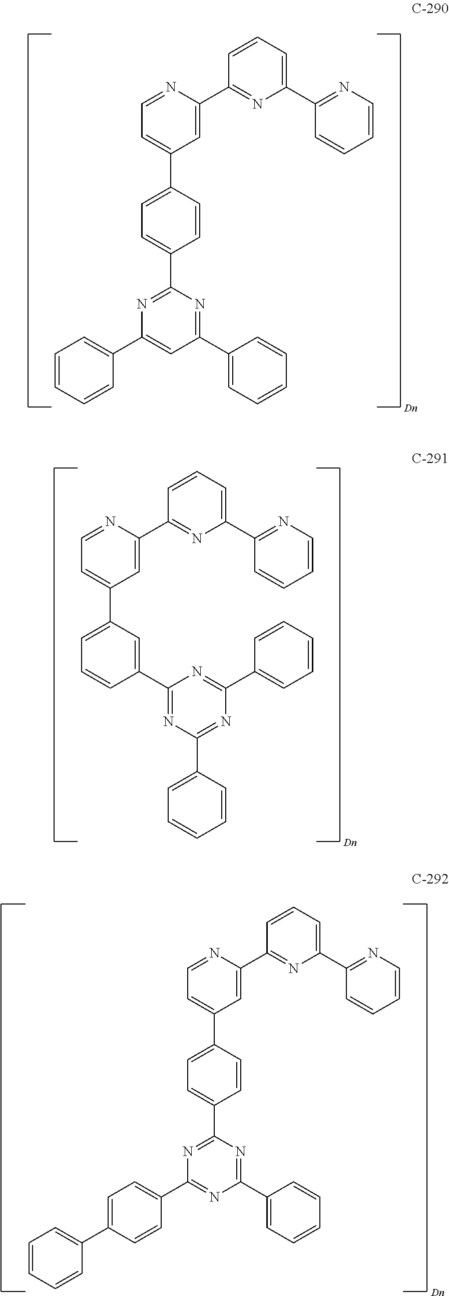

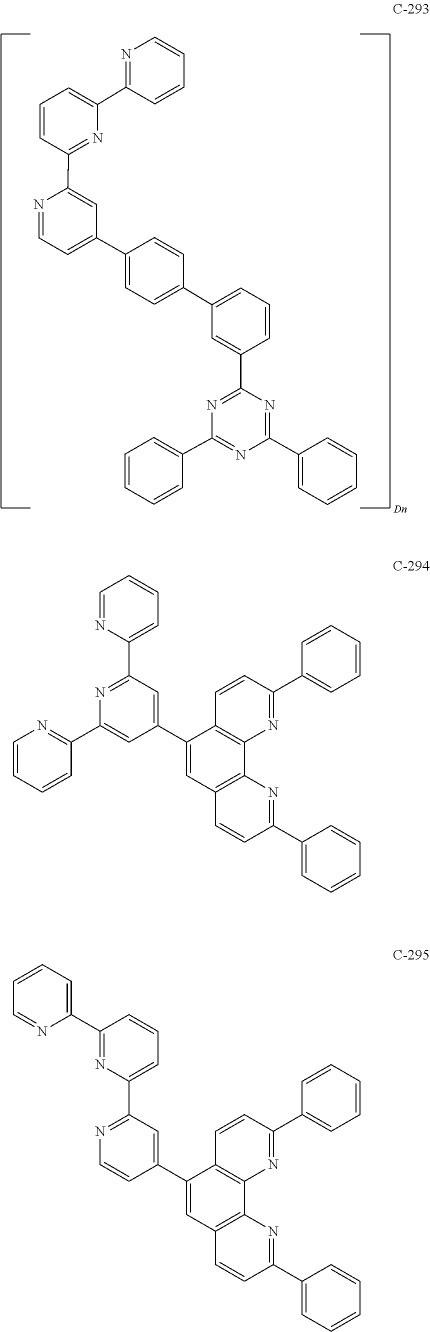

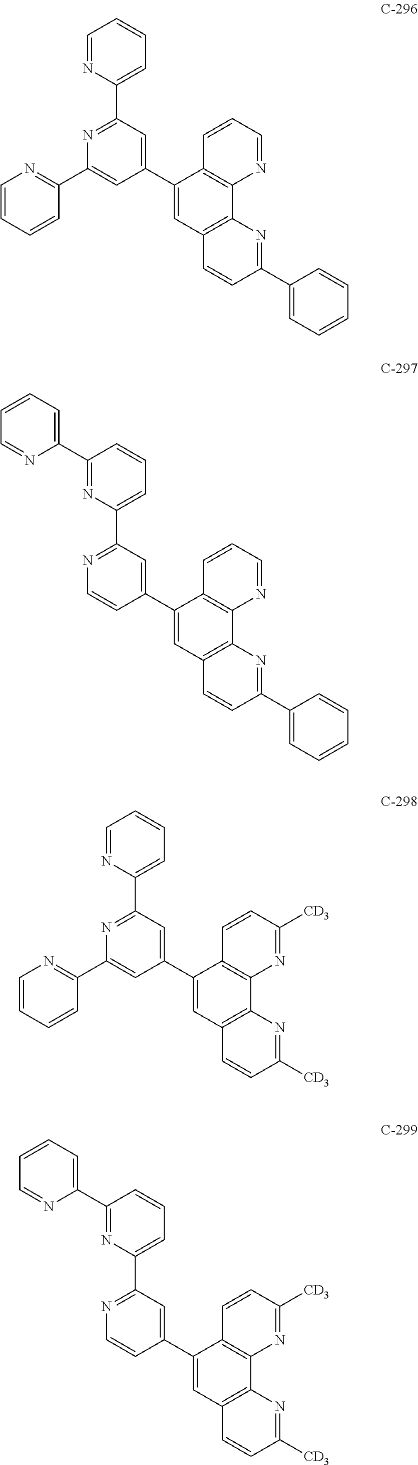 [SMILES: c1ccc(-c2ccc3cc(-c4cc(-c5ccccn5)nc(-c5ccccn5)c4)c4cccnc4c3n2)cc1 ; c1ccc(-c2ccc3cc(-c4ccnc(-c5cccc(-c6ccccn6)n5)c4)c4cccnc4c3n2)cc1 ; [2H]C([2H])([2H])c1ccc2cc(-c3cc(-c4ccccn4)nc(-c4ccccn4)c3)c3ccc(C([2H])([2H])[2H])nc3c2n1 ; [2H]C([2H])([2H])c1ccc2cc(-c3ccnc(-c4cccc(-c5ccccn5)n4)c3)c3ccc(C([2H])([2H])[2H])nc3c2n1]

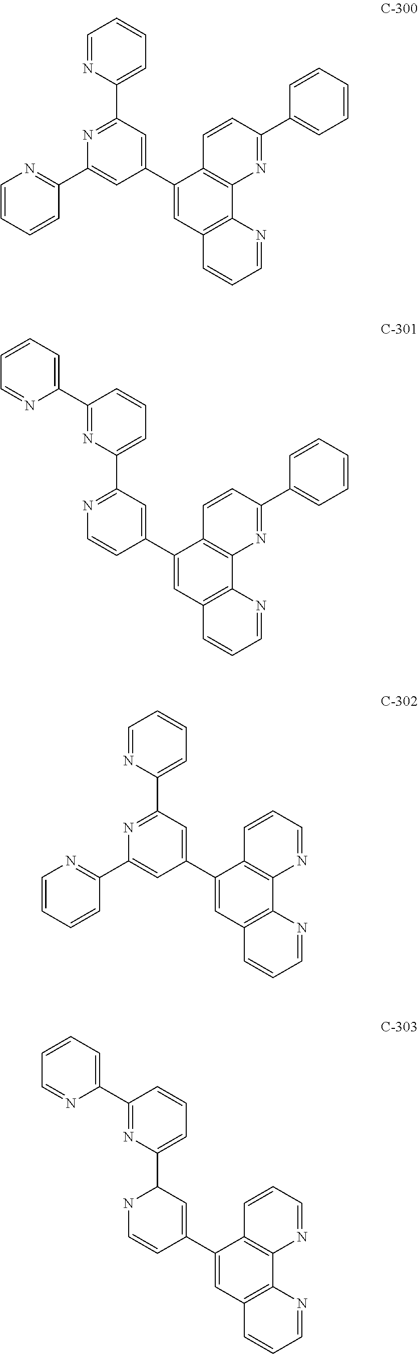

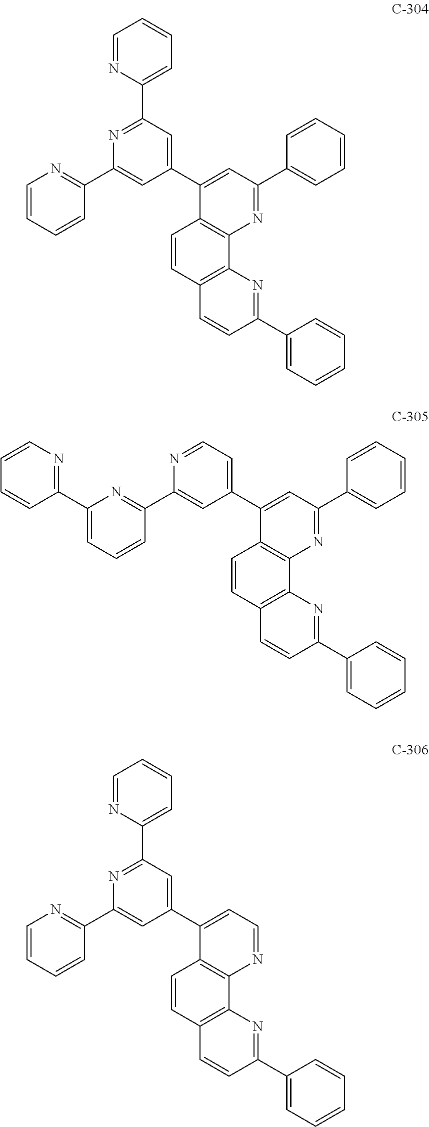

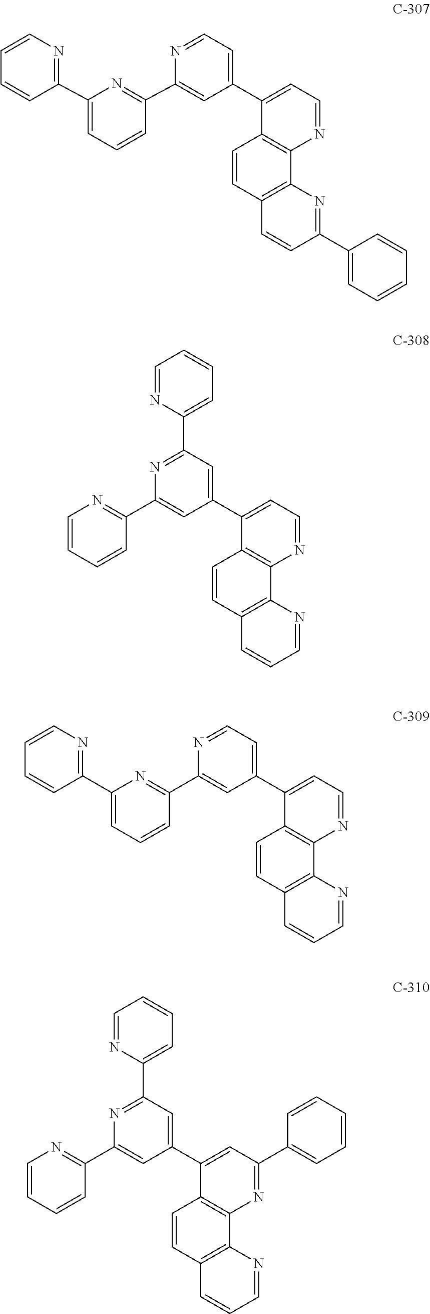

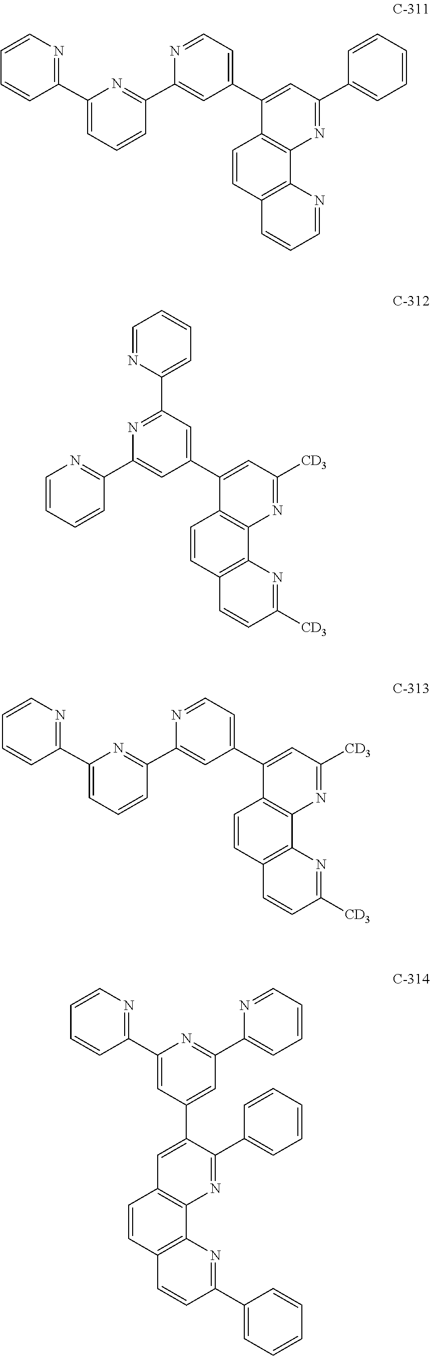 [SMILES: c1ccc(-c2cc(-c3ccnc(-c4cccc(-c5ccccn5)n4)c3)c3ccc4cccnc4c3n2)cc1 ; [2H]C([2H])([2H])c1ccc2ccc3c(-c4cc(-c5ccccn5)nc(-c5ccccn5)c4)cc(C([2H])([2H])[2H])nc3c2n1 ; [2H]C([2H])([2H])c1ccc2ccc3c(-c4ccnc(-c5cccc(-c6ccccn6)n5)c4)cc(C([2H])([2H])[2H])nc3c2n1 ; c1ccc(-c2ccc3ccc4cc(-c5cc(-c6ccccn6)nc(-c6ccccn6)c5)c(-c5ccccc5)nc4c3n2)cc1]

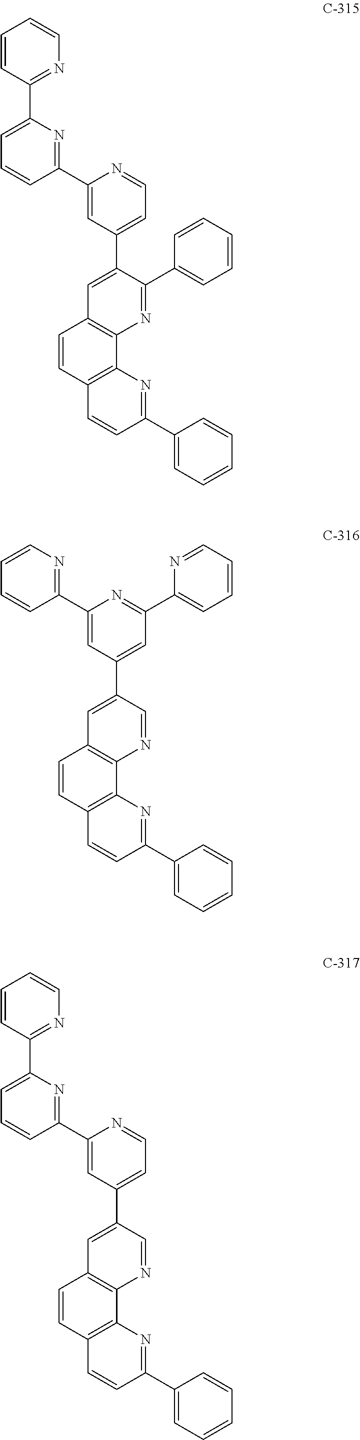 [SMILES: c1ccc(-c2ccc3ccc4cc(-c5ccnc(-c6cccc(-c7ccccn7)n6)c5)c(-c5ccccc5)nc4c3n2)cc1 ; c1ccc(-c2ccc3ccc4cc(-c5cc(-c6ccccn6)nc(-c6ccccn6)c5)cnc4c3n2)cc1 ; c1ccc(-c2ccc3ccc4cc(-c5ccnc(-c6cccc(-c7ccccn7)n6)c5)cnc4c3n2)cc1]

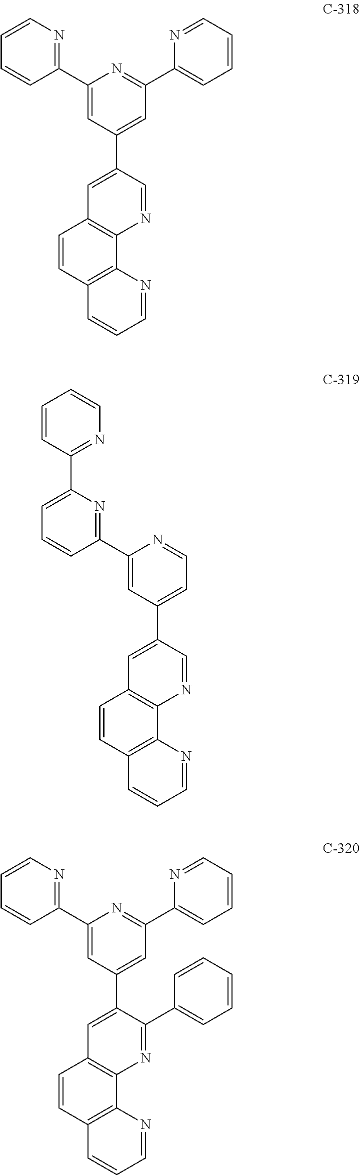 [SMILES: c1ccc(-c2cc(-c3cnc4c(ccc5cccnc54)c3)cc(-c3ccccn3)n2)nc1 ; c1ccc(-c2cccc(-c3cc(-c4cnc5c(ccc6cccnc65)c4)ccn3)n2)nc1 ; c1ccc(-c2nc3c(ccc4cccnc43)cc2-c2cc(-c3ccccn3)nc(-c3ccccn3)c2)cc1]

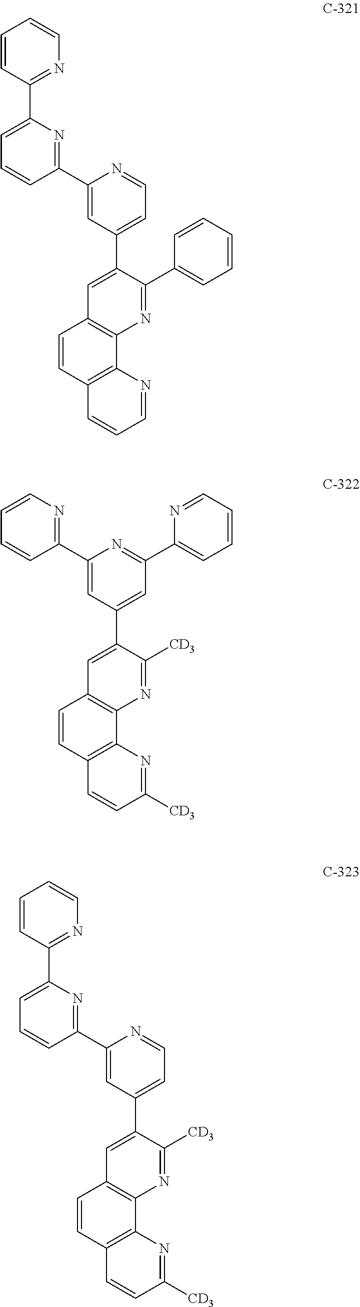

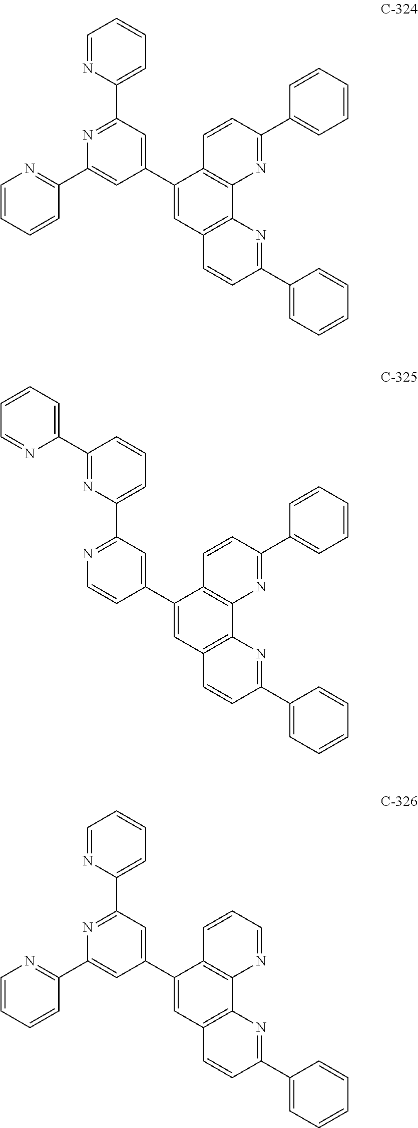 [SMILES: c1ccc(-c2ccc3cc(-c4cc(-c5ccccn5)nc(-c5ccccn5)c4)c4ccc(-c5ccccc5)nc4c3n2)cc1 ; c1ccc(-c2ccc3cc(-c4ccnc(-c5cccc(-c6ccccn6)n5)c4)c4ccc(-c5ccccc5)nc4c3n2)cc1 ; c1ccc(-c2ccc3cc(-c4cc(-c5ccccn5)nc(-c5ccccn5)c4)c4cccnc4c3n2)cc1]

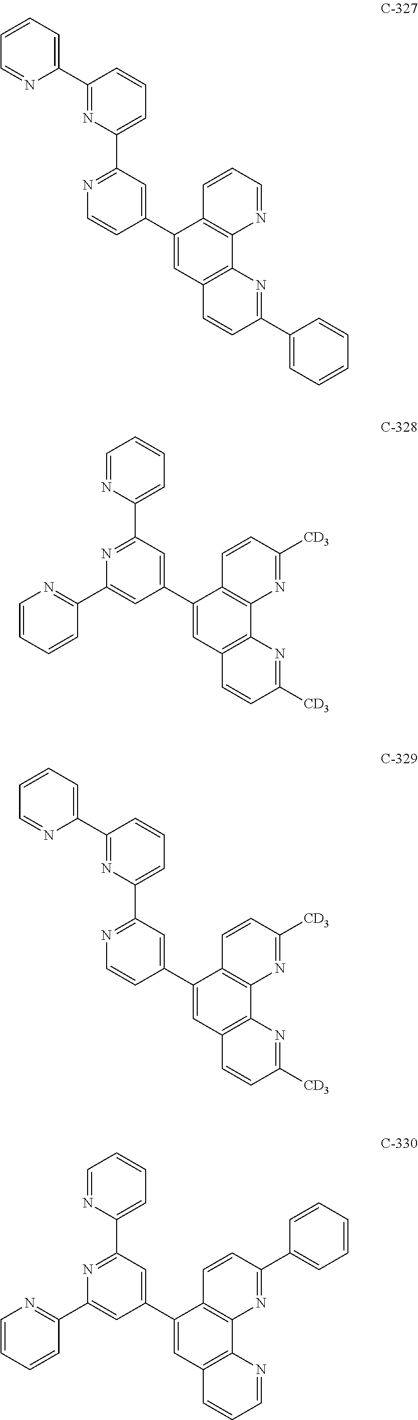 [SMILES: c1ccc(-c2ccc3cc(-c4ccnc(-c5cccc(-c6ccccn6)n5)c4)c4cccnc4c3n2)cc1 ; [2H]C([2H])([2H])c1ccc2cc(-c3cc(-c4ccccn4)nc(-c4ccccn4)c3)c3ccc(C([2H])([2H])[2H])nc3c2n1 ; [2H]C([2H])([2H])c1ccc2cc(-c3ccnc(-c4cccc(-c5ccccn5)n4)c3)c3ccc(C([2H])([2H])[2H])nc3c2n1 ; c1ccc(-c2ccc3c(-c4cc(-c5ccccn5)nc(-c5ccccn5)c4)cc4cccnc4c3n2)cc1]

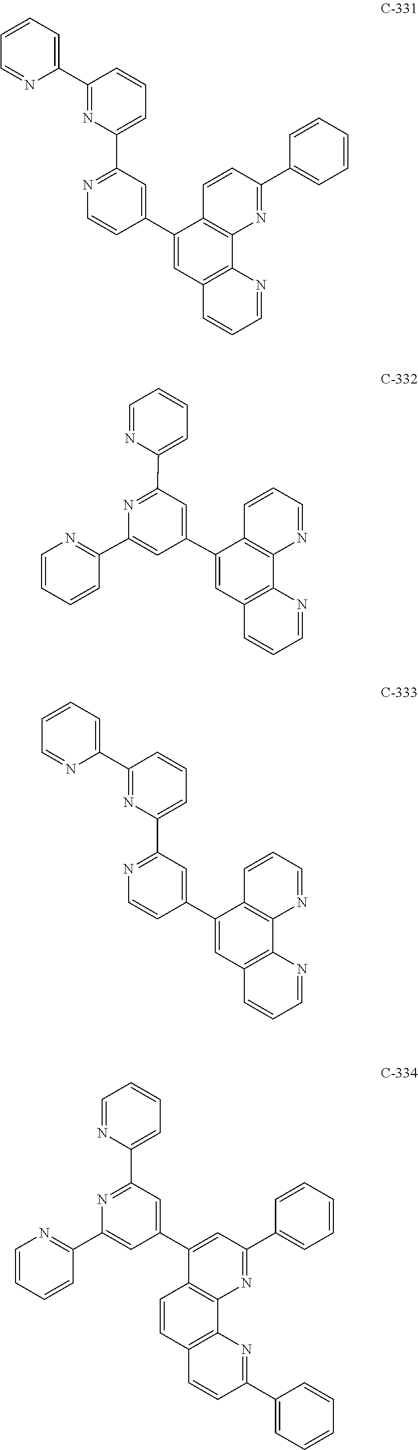

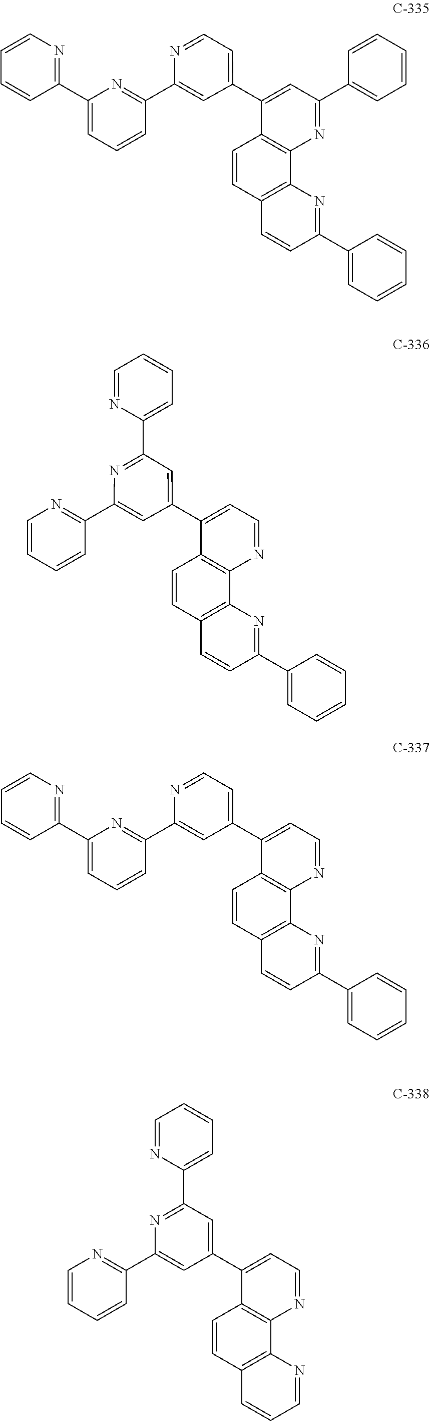

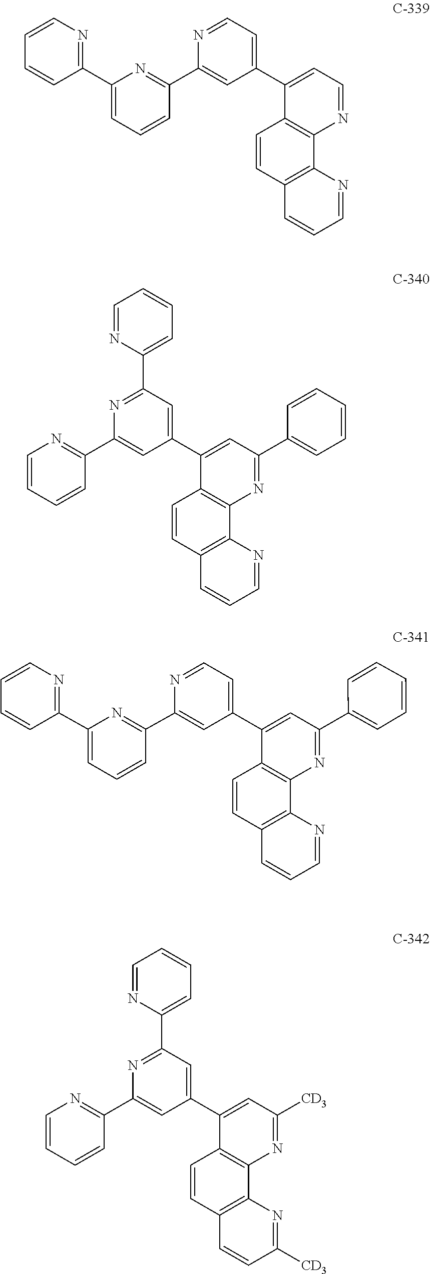

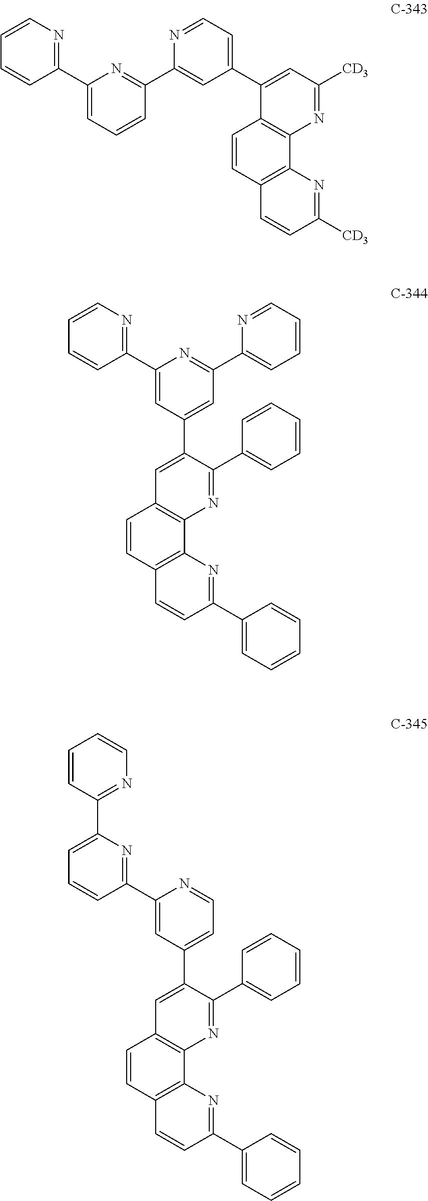 [SMILES: [2H]C([2H])([2H])c1ccc2ccc3c(-c4ccnc(-c5cccc(-c6ccccn6)n5)c4)cc(C([2H])([2H])[2H])nc3c2n1 ; c1ccc(-c2ccc3ccc4cc(-c5cc(-c6ccccn6)nc(-c6ccccn6)c5)c(-c5ccccc5)nc4c3n2)cc1 ; c1ccc(-c2ccc3ccc4cc(-c5ccnc(-c6cccc(-c7ccccn7)n6)c5)c(-c5ccccc5)nc4c3n2)cc1]

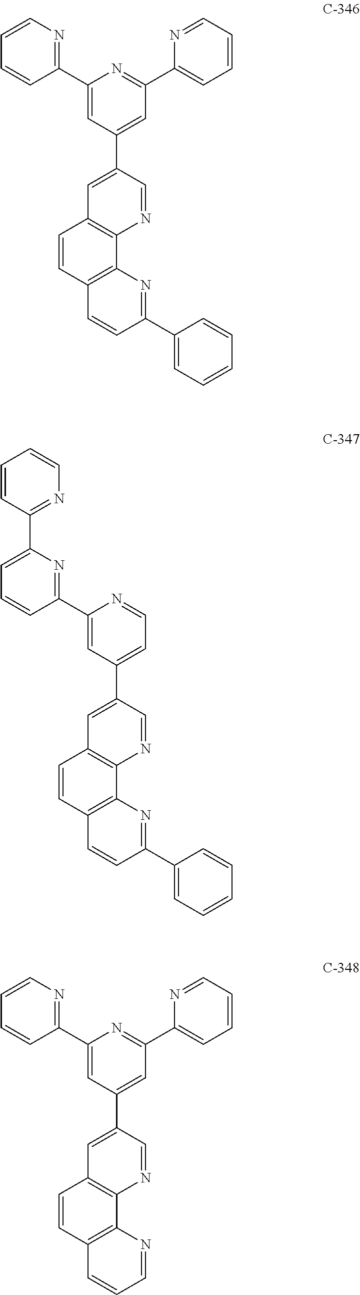 [SMILES: c1ccc(-c2ccc3ccc4cc(-c5cc(-c6ccccn6)nc(-c6ccccn6)c5)cnc4c3n2)cc1 ; c1ccc(-c2ccc3ccc4cc(-c5ccnc(-c6cccc(-c7ccccn7)n6)c5)cnc4c3n2)cc1 ; c1ccc(-c2cc(-c3cnc4c(ccc5cccnc54)c3)cc(-c3ccccn3)n2)nc1]

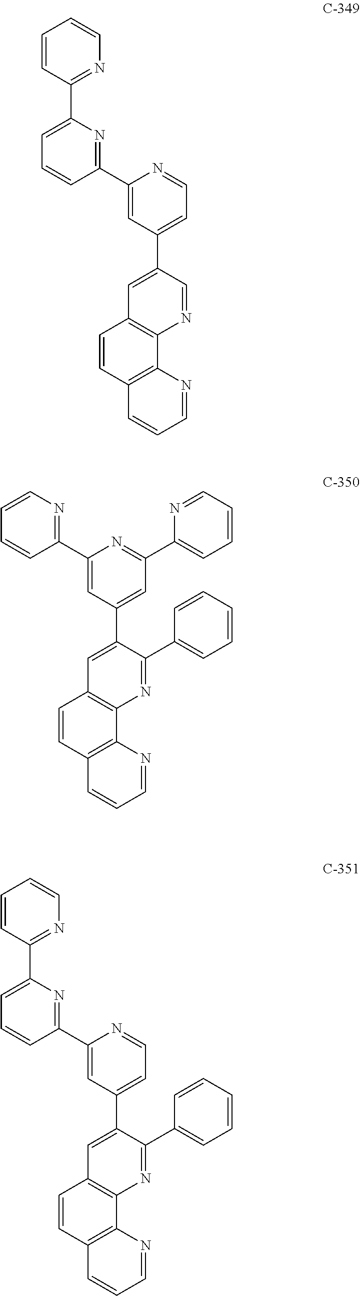 [SMILES: c1ccc(-c2cccc(-c3cc(-c4cnc5c(ccc6cccnc65)c4)ccn3)n2)nc1 ; c1ccc(-c2nc3c(ccc4cccnc43)cc2-c2cc(-c3ccccn3)nc(-c3ccccn3)c2)cc1 ; c1ccc(-c2nc3c(ccc4cccnc43)cc2-c2ccnc(-c3cccc(-c4ccccn4)n3)c2)cc1]

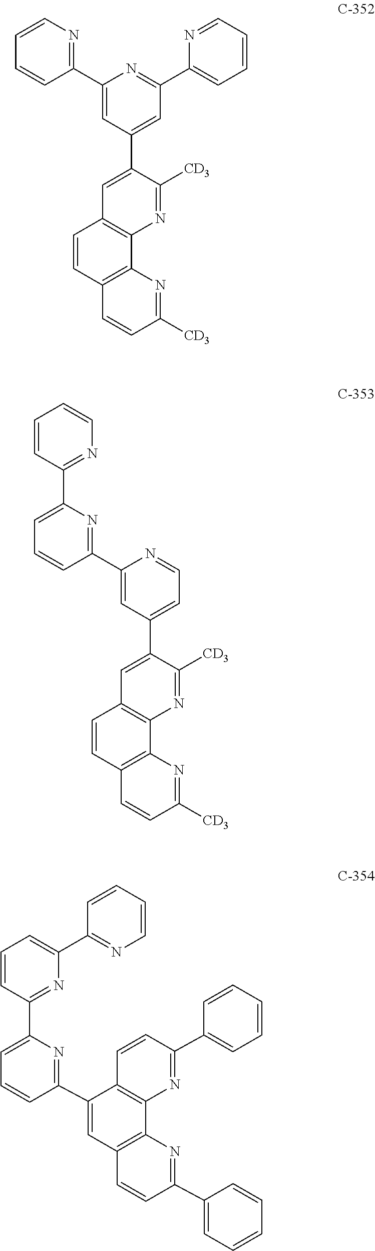 [SMILES: [2H]C([2H])([2H])c1ccc2ccc3cc(-c4cc(-c5ccccn5)nc(-c5ccccn5)c4)c(C([2H])([2H])[2H])nc3c2n1 ; [2H]C([2H])([2H])c1ccc2ccc3cc(-c4ccnc(-c5cccc(-c6ccccn6)n5)c4)c(C([2H])([2H])[2H])nc3c2n1 ; c1ccc(-c2ccc3cc(-c4cccc(-c5cccc(-c6ccccn6)n5)n4)c4ccc(-c5ccccc5)nc4c3n2)cc1]

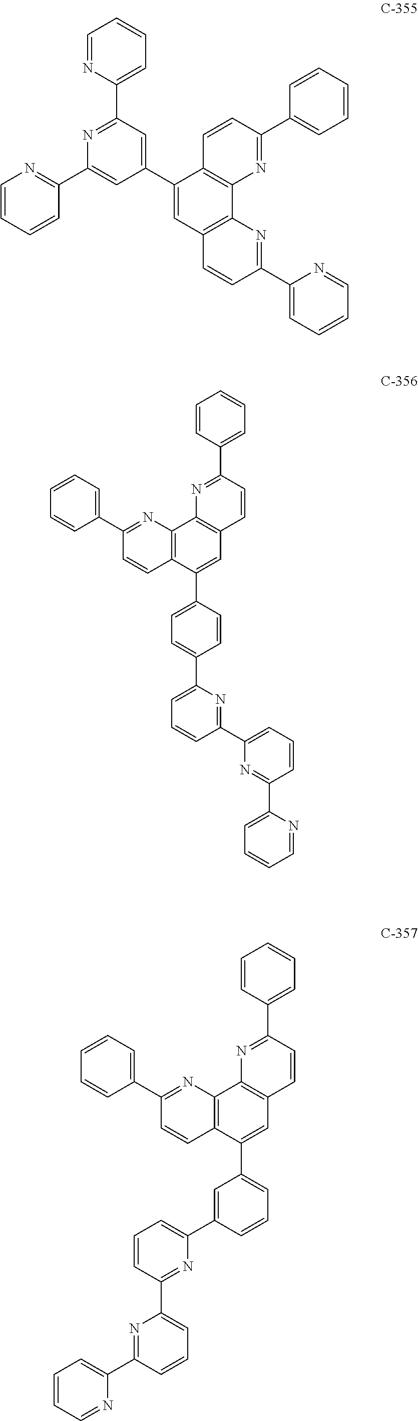 [SMILES: c1ccc(-c2ccc3c(-c4cc(-c5ccccn5)nc(-c5ccccn5)c4)cc4ccc(-c5ccccn5)nc4c3n2)cc1 ; c1ccc(-c2ccc3cc(-c4ccc(-c5cccc(-c6cccc(-c7ccccn7)n6)n5)cc4)c4ccc(-c5ccccc5)nc4c3n2)cc1 ; c1ccc(-c2ccc3cc(-c4cccc(-c5cccc(-c6cccc(-c7ccccn7)n6)n5)c4)c4ccc(-c5ccccc5)nc4c3n2)cc1]

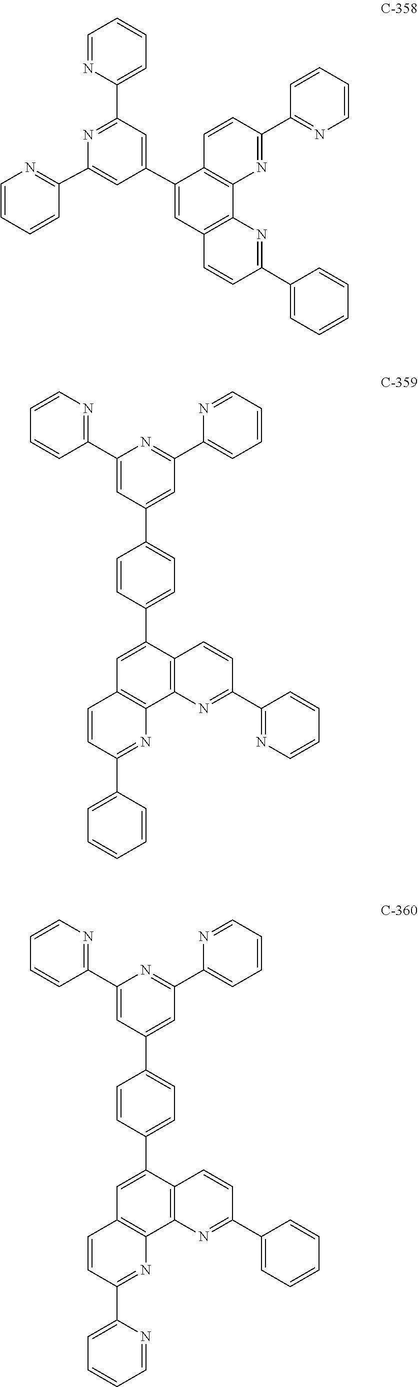 [SMILES: c1ccc(-c2ccc3cc(-c4cc(-c5ccccn5)nc(-c5ccccn5)c4)c4ccc(-c5ccccn5)nc4c3n2)cc1 ; c1ccc(-c2ccc3cc(-c4ccc(-c5cc(-c6ccccn6)nc(-c6ccccn6)c5)cc4)c4ccc(-c5ccccn5)nc4c3n2)cc1 ; c1ccc(-c2ccc3c(-c4ccc(-c5cc(-c6ccccn6)nc(-c6ccccn6)c5)cc4)cc4ccc(-c5ccccn5)nc4c3n2)cc1]

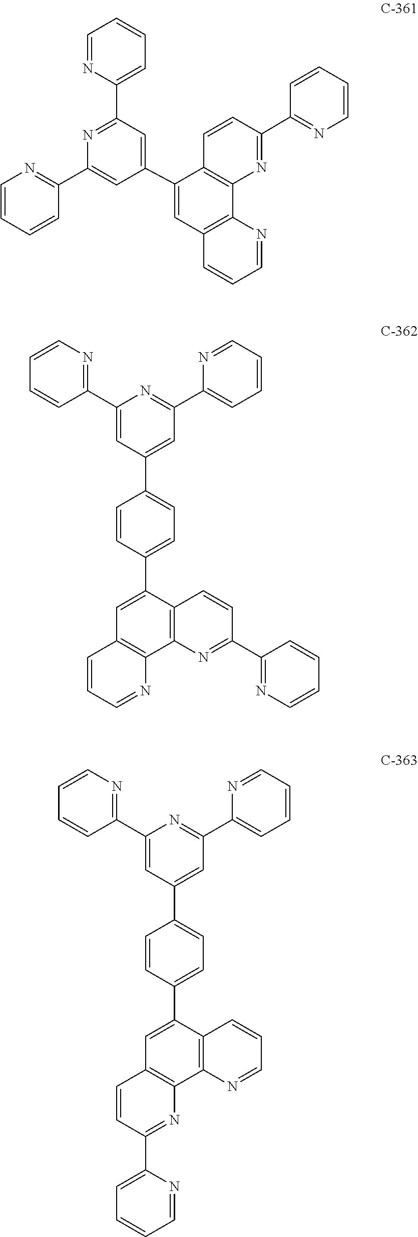 [SMILES: c1ccc(-c2cc(-c3cc4cccnc4c4nc(-c5ccccn5)ccc34)cc(-c3ccccn3)n2)nc1 ; c1ccc(-c2cc(-c3ccc(-c4cc5cccnc5c5nc(-c6ccccn6)ccc45)cc3)cc(-c3ccccn3)n2)nc1 ; c1ccc(-c2cc(-c3ccc(-c4cc5ccc(-c6ccccn6)nc5c5ncccc45)cc3)cc(-c3ccccn3)n2)nc1]

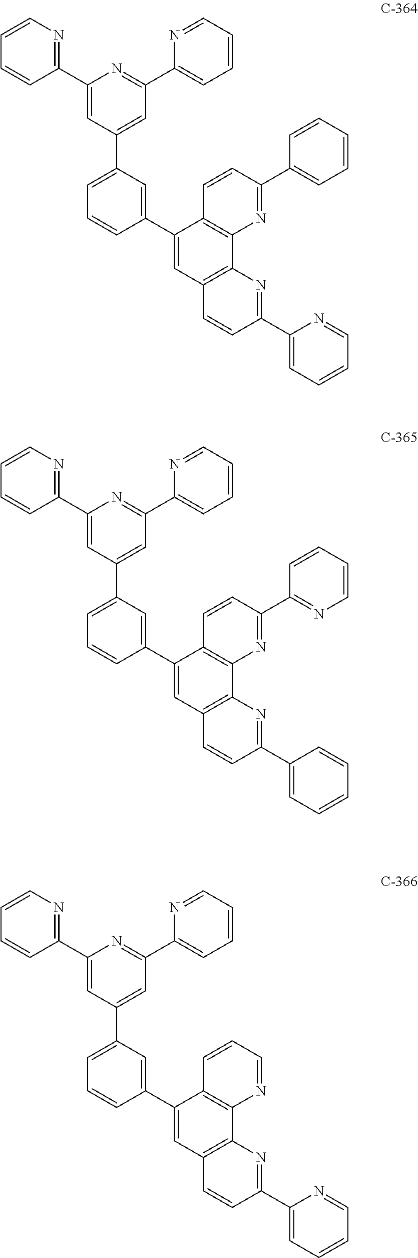 [SMILES: c1ccc(-c2ccc3c(-c4cccc(-c5cc(-c6ccccn6)nc(-c6ccccn6)c5)c4)cc4ccc(-c5ccccn5)nc4c3n2)cc1 ; c1ccc(-c2ccc3cc(-c4cccc(-c5cc(-c6ccccn6)nc(-c6ccccn6)c5)c4)c4ccc(-c5ccccn5)nc4c3n2)cc1 ; c1ccc(-c2cc(-c3cccc(-c4cc5ccc(-c6ccccn6)nc5c5ncccc45)c3)cc(-c3ccccn3)n2)nc1]

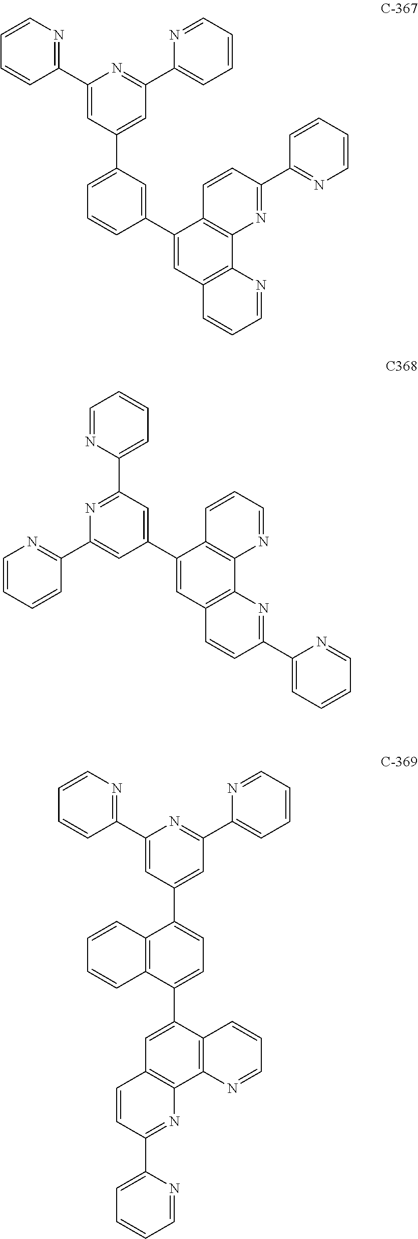 [SMILES: c1ccc(-c2cc(-c3cccc(-c4cc5cccnc5c5nc(-c6ccccn6)ccc45)c3)cc(-c3ccccn3)n2)nc1 ; c1ccc(-c2cc(-c3cc4ccc(-c5ccccn5)nc4c4ncccc34)cc(-c3ccccn3)n2)nc1 ; c1ccc(-c2cc(-c3ccc(-c4cc5ccc(-c6ccccn6)nc5c5ncccc45)c4ccccc34)cc(-c3ccccn3)n2)nc1]

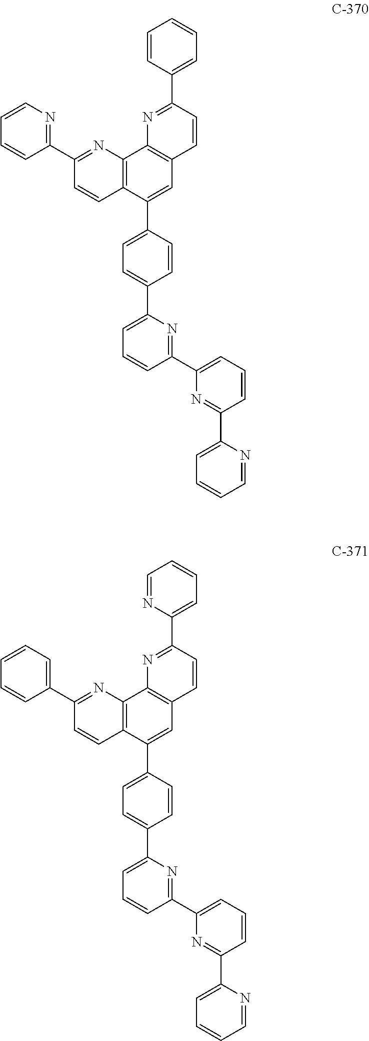 [SMILES: c1ccc(-c2ccc3cc(-c4ccc(-c5cccc(-c6cccc(-c7ccccn7)n6)n5)cc4)c4ccc(-c5ccccn5)nc4c3n2)cc1 ; c1ccc(-c2ccc3c(-c4ccc(-c5cccc(-c6cccc(-c7ccccn7)n6)n5)cc4)cc4ccc(-c5ccccn5)nc4c3n2)cc1]

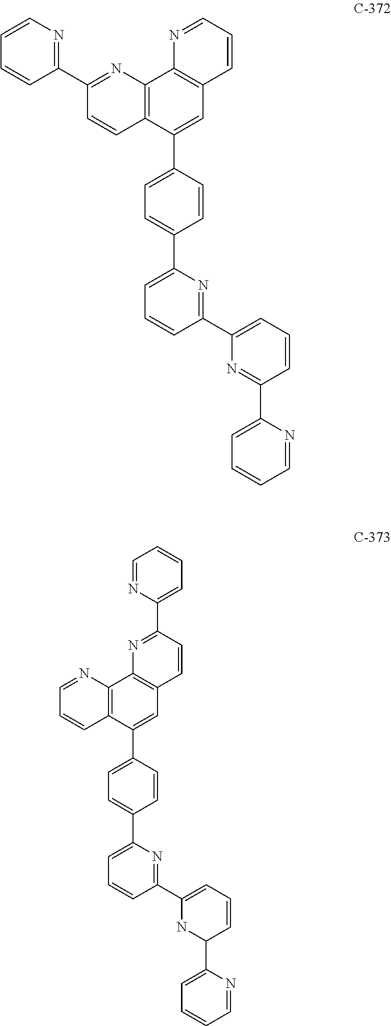

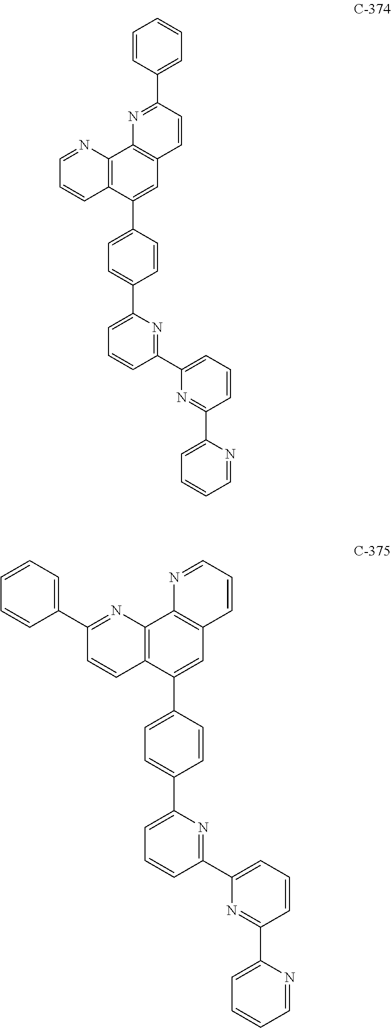 [SMILES: c1ccc(-c2ccc3cc(-c4ccc(-c5cccc(-c6cccc(-c7ccccn7)n6)n5)cc4)c4cccnc4c3n2)cc1 ; c1ccc(-c2ccc3c(-c4ccc(-c5cccc(-c6cccc(-c7ccccn7)n6)n5)cc4)cc4cccnc4c3n2)cc1]

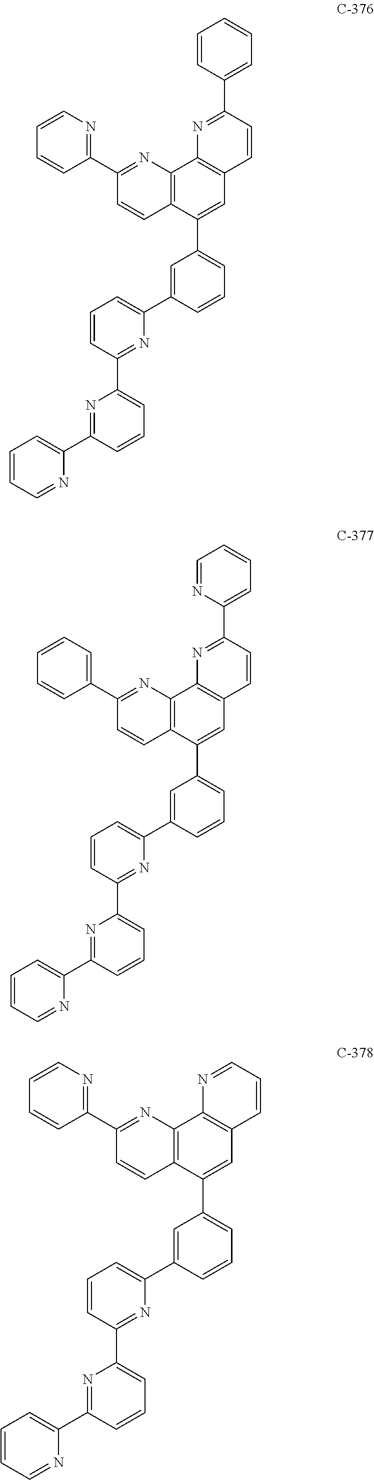 [SMILES: c1ccc(-c2ccc3cc(-c4cccc(-c5cccc(-c6cccc(-c7ccccn7)n6)n5)c4)c4ccc(-c5ccccn5)nc4c3n2)cc1 ; c1ccc(-c2ccc3c(-c4cccc(-c5cccc(-c6cccc(-c7ccccn7)n6)n5)c4)cc4ccc(-c5ccccn5)nc4c3n2)cc1 ; c1ccc(-c2cccc(-c3cccc(-c4cccc(-c5cc6cccnc6c6nc(-c7ccccn7)ccc56)c4)n3)n2)nc1]

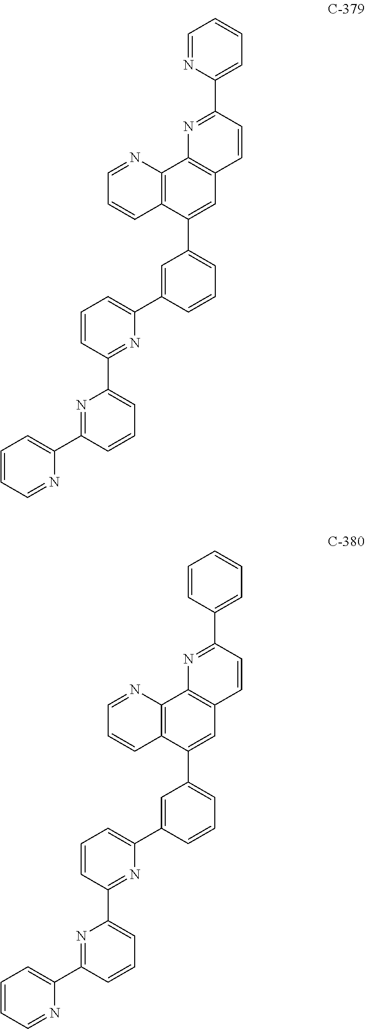 [SMILES: c1ccc(-c2cccc(-c3cccc(-c4cccc(-c5cc6ccc(-c7ccccn7)nc6c6ncccc56)c4)n3)n2)nc1 ; c1ccc(-c2ccc3cc(-c4cccc(-c5cccc(-c6cccc(-c7ccccn7)n6)n5)c4)c4cccnc4c3n2)cc1]

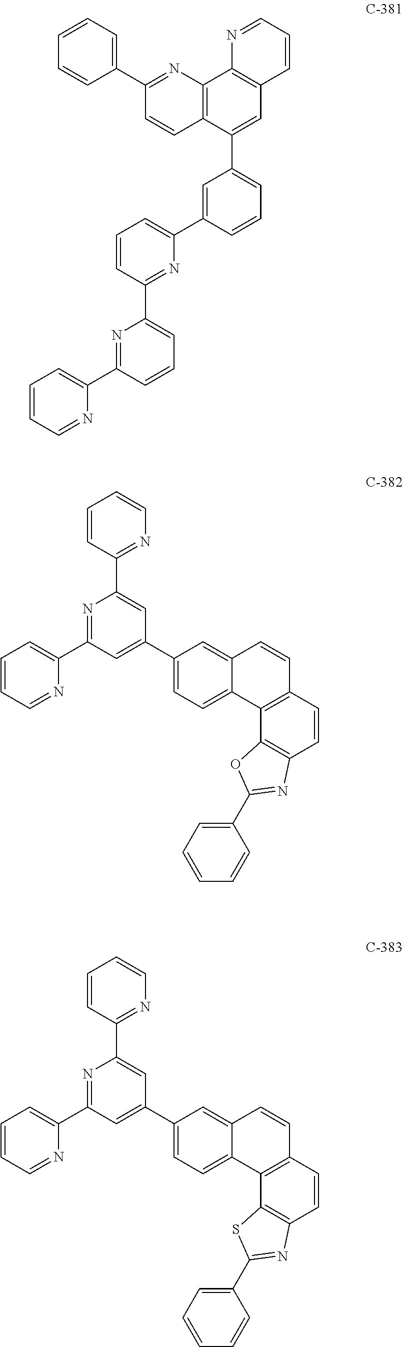 [SMILES: c1ccc(-c2ccc3c(-c4cccc(-c5cccc(-c6cccc(-c7ccccn7)n6)n5)c4)cc4cccnc4c3n2)cc1 ; c1ccc(-c2nc3ccc4ccc5cc(-c6cc(-c7ccccn7)nc(-c7ccccn7)c6)ccc5c4c3o2)cc1 ; c1ccc(-c2nc3ccc4ccc5cc(-c6cc(-c7ccccn7)nc(-c7ccccn7)c6)ccc5c4c3s2)cc1]

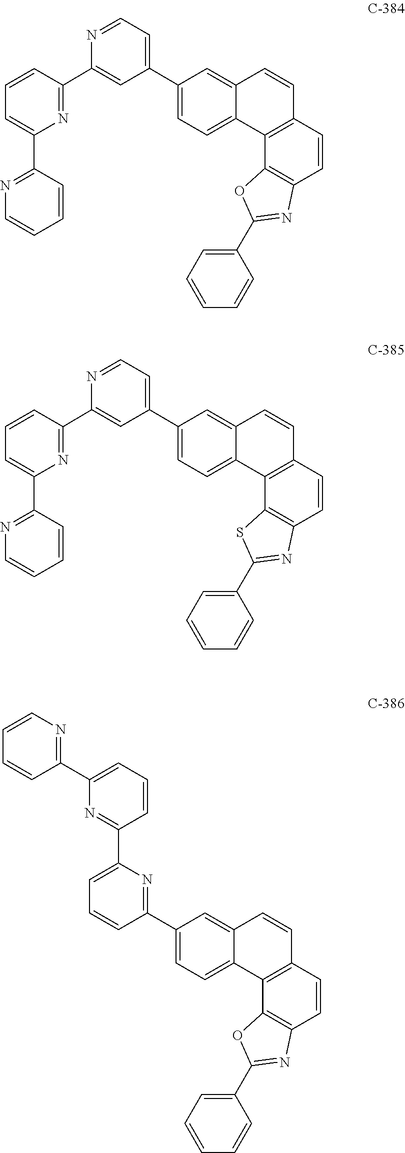 [SMILES: c1ccc(-c2nc3ccc4ccc5cc(-c6ccnc(-c7cccc(-c8ccccn8)n7)c6)ccc5c4c3o2)cc1 ; c1ccc(-c2nc3ccc4ccc5cc(-c6ccnc(-c7cccc(-c8ccccn8)n7)c6)ccc5c4c3s2)cc1 ; c1ccc(-c2nc3ccc4ccc5cc(-c6cccc(-c7cccc(-c8ccccn8)n7)n6)ccc5c4c3o2)cc1]

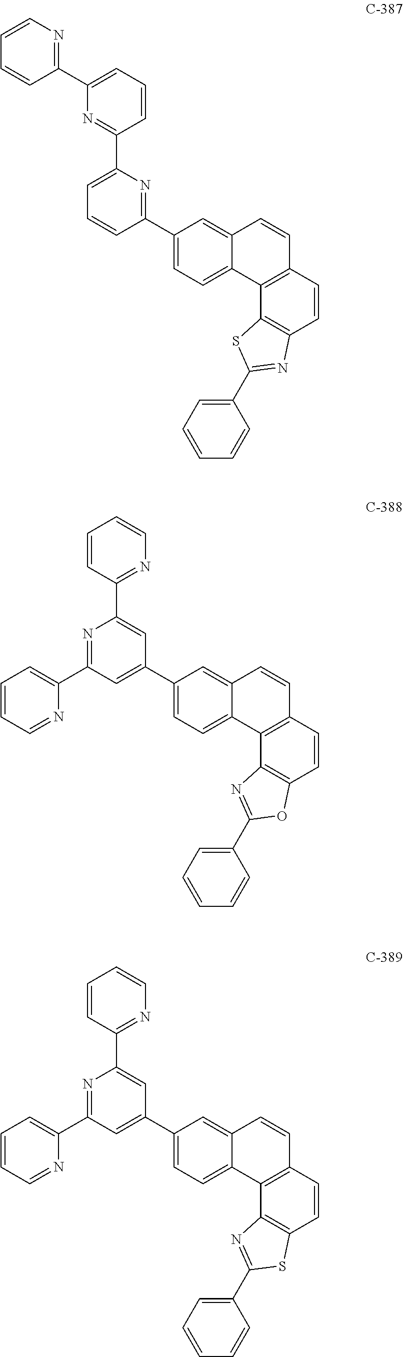 [SMILES: c1ccc(-c2nc3ccc4ccc5cc(-c6cccc(-c7cccc(-c8ccccn8)n7)n6)ccc5c4c3s2)cc1 ; c1ccc(-c2nc3c(ccc4ccc5cc(-c6cc(-c7ccccn7)nc(-c7ccccn7)c6)ccc5c43)o2)cc1 ; c1ccc(-c2nc3c(ccc4ccc5cc(-c6cc(-c7ccccn7)nc(-c7ccccn7)c6)ccc5c43)s2)cc1]

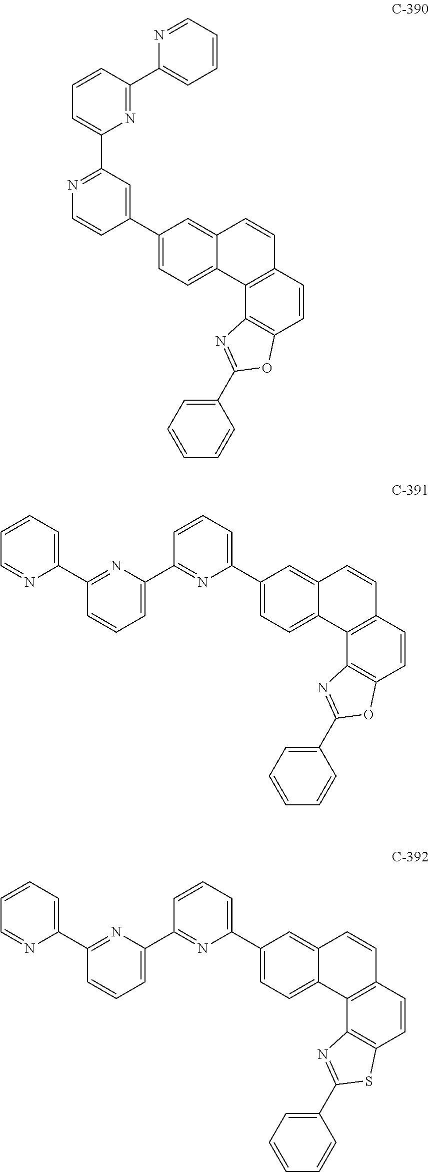 [SMILES: c1ccc(-c2nc3c(ccc4ccc5cc(-c6ccnc(-c7cccc(-c8ccccn8)n7)c6)ccc5c43)o2)cc1 ; c1ccc(-c2nc3c(ccc4ccc5cc(-c6cccc(-c7cccc(-c8ccccn8)n7)n6)ccc5c43)o2)cc1 ; c1ccc(-c2nc3c(ccc4ccc5cc(-c6cccc(-c7cccc(-c8ccccn8)n7)n6)ccc5c43)s2)cc1]

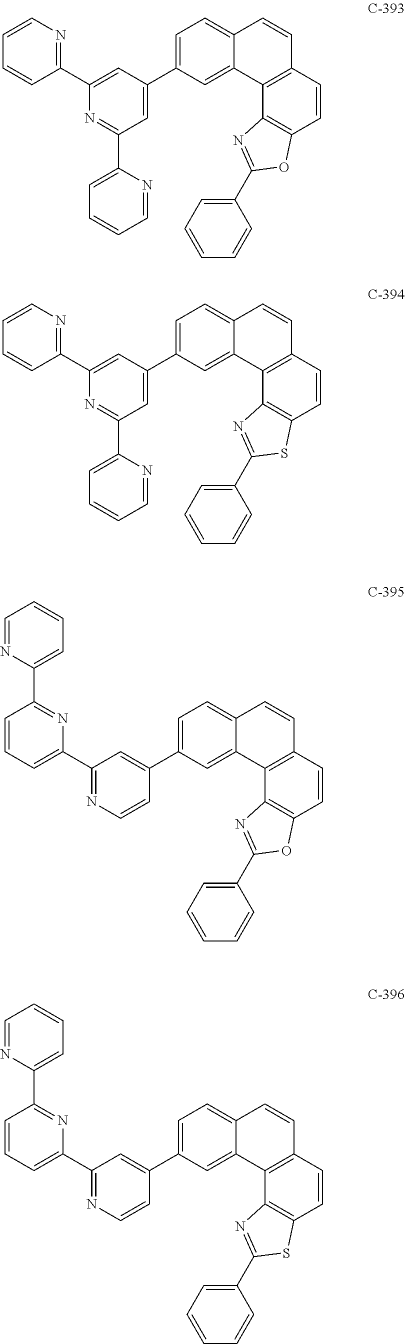 [SMILES: c1ccc(-c2nc3c(ccc4ccc5ccc(-c6cc(-c7ccccn7)nc(-c7ccccn7)c6)cc5c43)o2)cc1 ; c1ccc(-c2nc3c(ccc4ccc5ccc(-c6cc(-c7ccccn7)nc(-c7ccccn7)c6)cc5c43)s2)cc1 ; c1ccc(-c2nc3c(ccc4ccc5ccc(-c6ccnc(-c7cccc(-c8ccccn8)n7)c6)cc5c43)o2)cc1 ; c1ccc(-c2nc3c(ccc4ccc5ccc(-c6ccnc(-c7cccc(-c8ccccn8)n7)c6)cc5c43)s2)cc1]

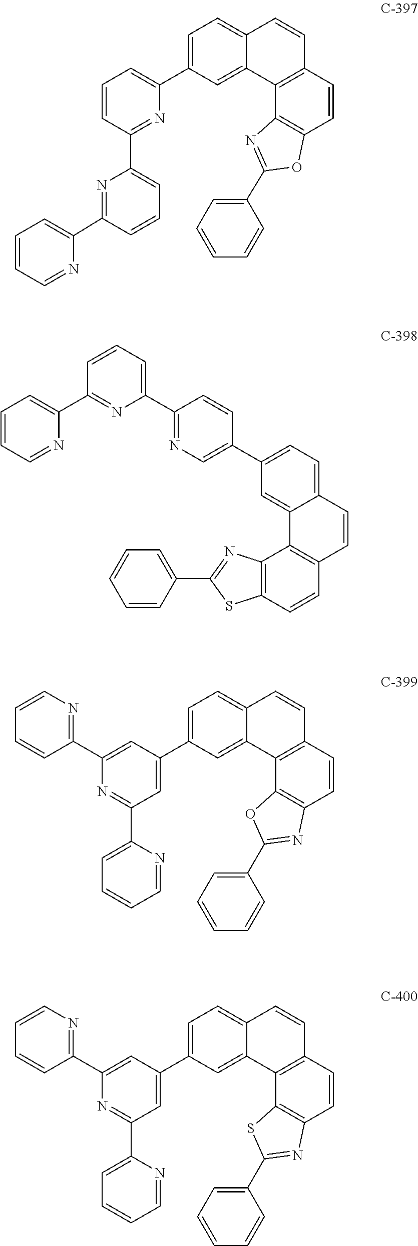 [SMILES: c1ccc(-c2nc3c(ccc4ccc5ccc(-c6cccc(-c7cccc(-c8ccccn8)n7)n6)cc5c43)o2)cc1 ; c1ccc(-c2nc3c(ccc4ccc5ccc(-c6ccc(-c7cccc(-c8ccccn8)n7)nc6)cc5c43)s2)cc1 ; c1ccc(-c2nc3ccc4ccc5ccc(-c6cc(-c7ccccn7)nc(-c7ccccn7)c6)cc5c4c3o2)cc1 ; c1ccc(-c2nc3ccc4ccc5ccc(-c6cc(-c7ccccn7)nc(-c7ccccn7)c6)cc5c4c3s2)cc1]

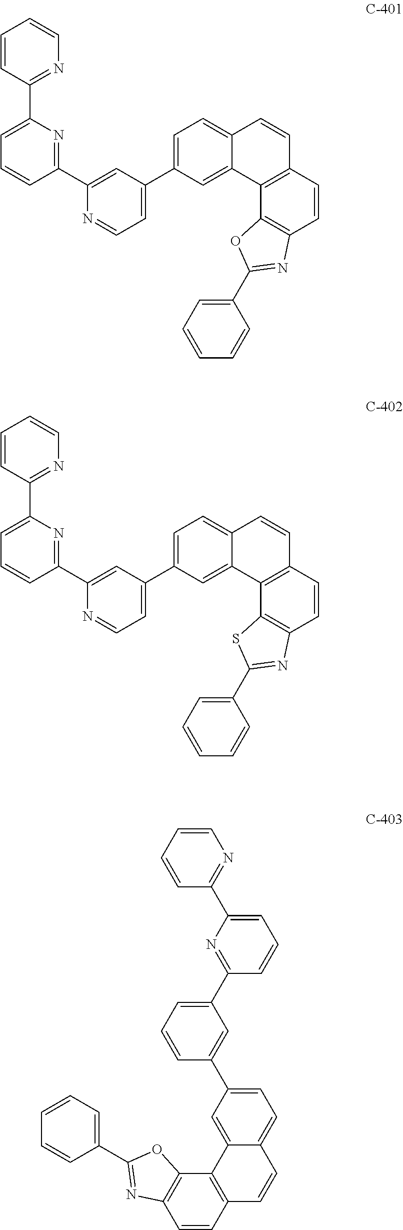 [SMILES: c1ccc(-c2nc3ccc4ccc5ccc(-c6ccnc(-c7cccc(-c8ccccn8)n7)c6)cc5c4c3o2)cc1 ; c1ccc(-c2nc3ccc4ccc5ccc(-c6ccnc(-c7cccc(-c8ccccn8)n7)c6)cc5c4c3s2)cc1 ; c1ccc(-c2nc3ccc4ccc5ccc(-c6cccc(-c7cccc(-c8ccccn8)n7)c6)cc5c4c3o2)cc1]

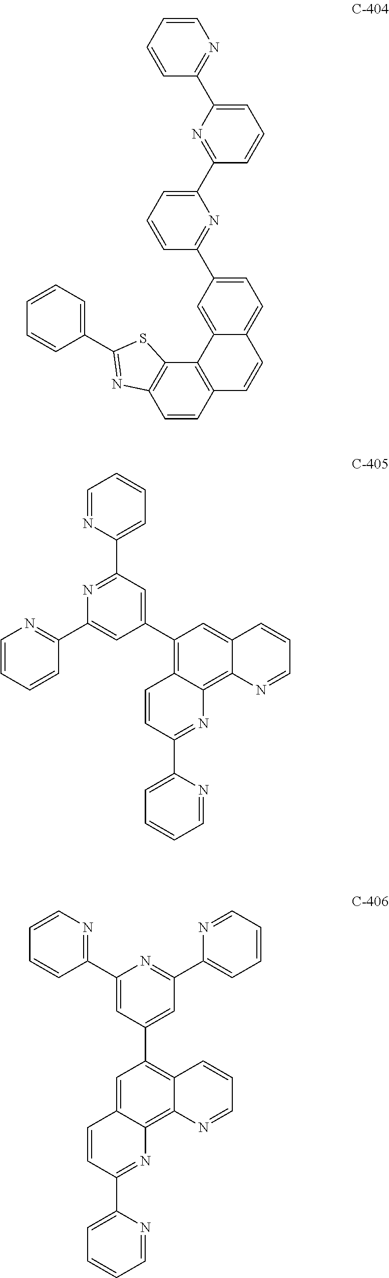 [SMILES: c1ccc(-c2nc3ccc4ccc5ccc(-c6cccc(-c7cccc(-c8ccccn8)n7)n6)cc5c4c3s2)cc1 ; c1ccc(-c2cc(-c3cc4cccnc4c4nc(-c5ccccn5)ccc34)cc(-c3ccccn3)n2)nc1 ; c1ccc(-c2cc(-c3cc4ccc(-c5ccccn5)nc4c4ncccc34)cc(-c3ccccn3)n2)nc1]

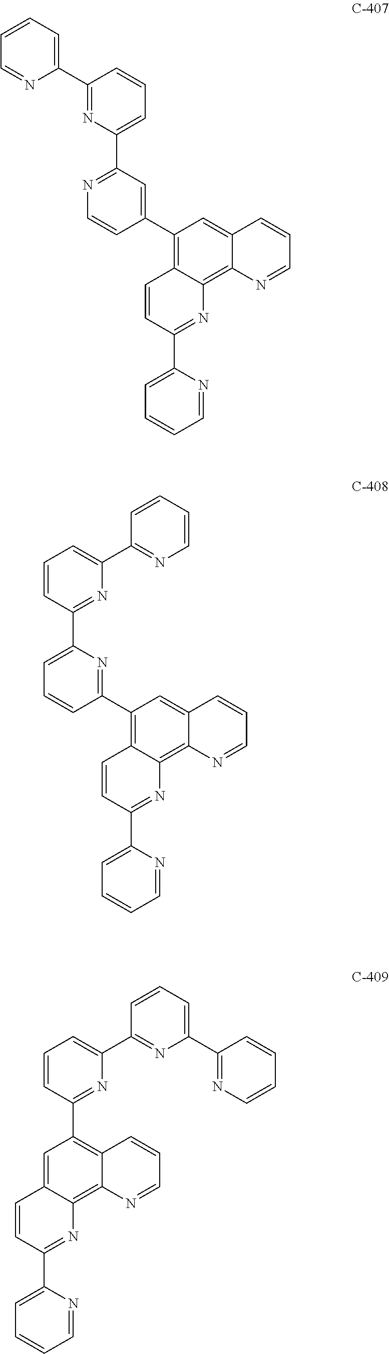 [SMILES: c1ccc(-c2cccc(-c3cc(-c4cc5cccnc5c5nc(-c6ccccn6)ccc45)ccn3)n2)nc1 ; c1ccc(-c2cccc(-c3cccc(-c4cc5cccnc5c5nc(-c6ccccn6)ccc45)n3)n2)nc1 ; c1ccc(-c2cccc(-c3cccc(-c4cc5ccc(-c6ccccn6)nc5c5ncccc45)n3)n2)nc1]

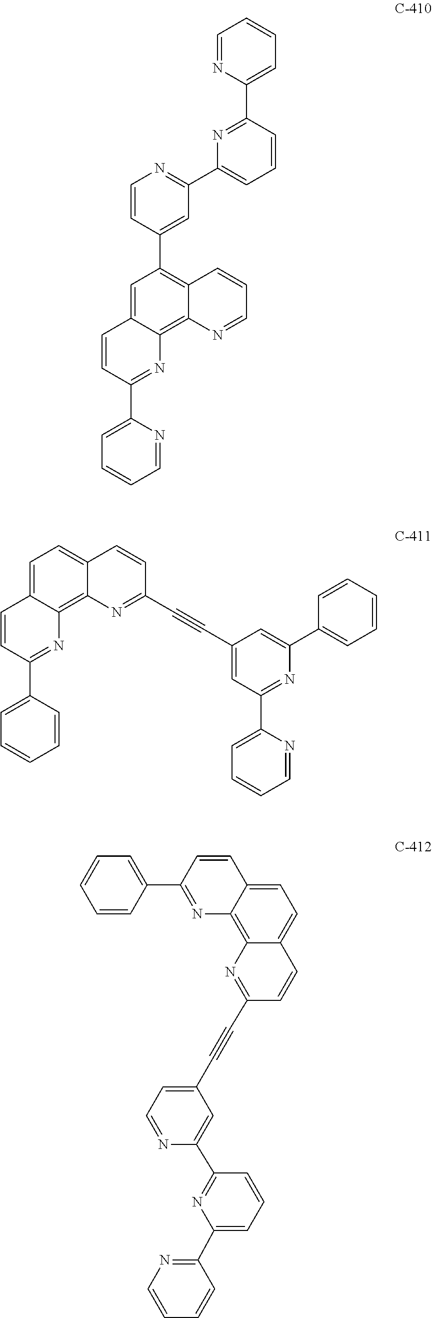 [SMILES: c1ccc(-c2cccc(-c3cc(-c4cc5ccc(-c6ccccn6)nc5c5ncccc45)ccn3)n2)nc1 ; C(#Cc1ccc2ccc3ccc(-c4ccccc4)nc3c2n1)c1cc(-c2ccccc2)nc(-c2ccccn2)c1 ; C(#Cc1ccc2ccc3ccc(-c4ccccc4)nc3c2n1)c1ccnc(-c2cccc(-c3ccccn3)n2)c1]

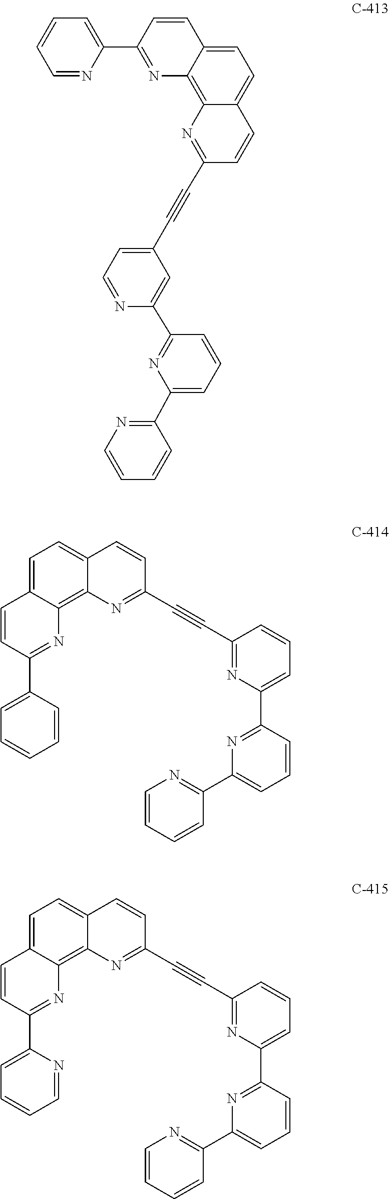 [SMILES: C(#Cc1ccc2ccc3ccc(-c4ccccn4)nc3c2n1)c1ccnc(-c2cccc(-c3ccccn3)n2)c1 ; C(#Cc1ccc2ccc3ccc(-c4ccccc4)nc3c2n1)c1cccc(-c2cccc(-c3ccccn3)n2)n1 ; C(#Cc1ccc2ccc3ccc(-c4ccccn4)nc3c2n1)c1cccc(-c2cccc(-c3ccccn3)n2)n1]

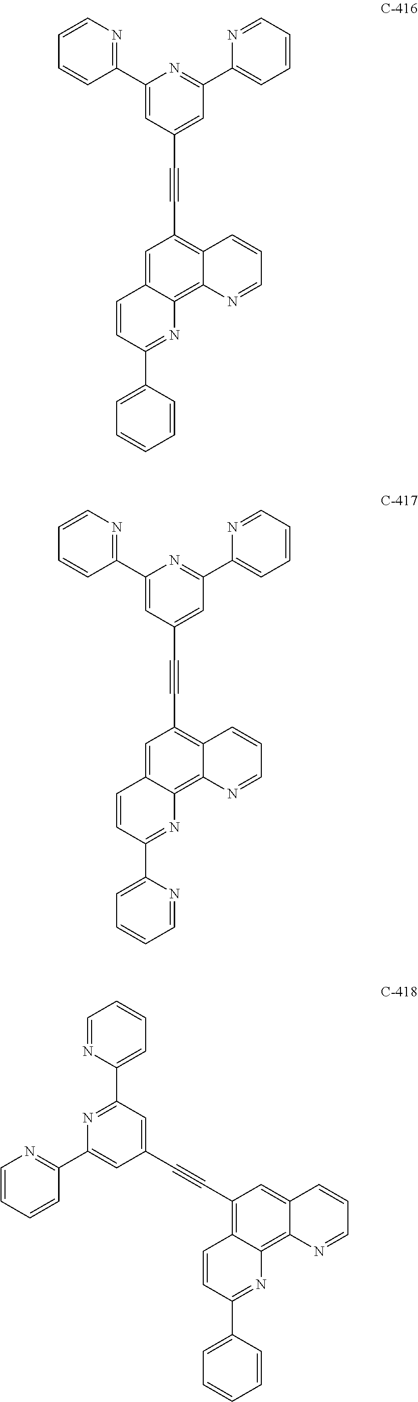 [SMILES: C(#Cc1cc2ccc(-c3ccccc3)nc2c2ncccc12)c1cc(-c2ccccn2)nc(-c2ccccn2)c1 ; C(#Cc1cc2ccc(-c3ccccn3)nc2c2ncccc12)c1cc(-c2ccccn2)nc(-c2ccccn2)c1 ; C(#Cc1cc2cccnc2c2nc(-c3ccccc3)ccc12)c1cc(-c2ccccn2)nc(-c2ccccn2)c1]

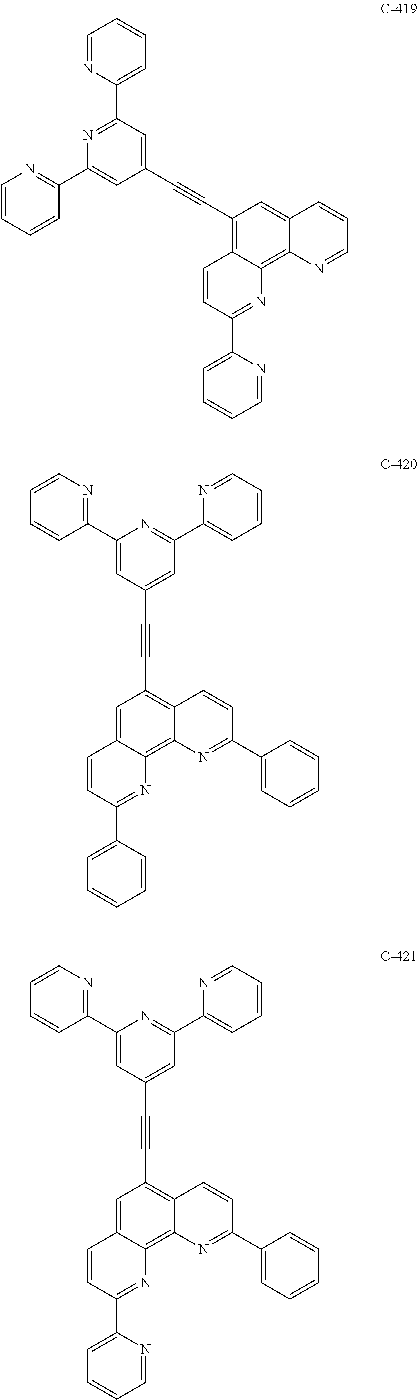 [SMILES: C(#Cc1cc2cccnc2c2nc(-c3ccccn3)ccc12)c1cc(-c2ccccn2)nc(-c2ccccn2)c1 ; C(#Cc1cc2ccc(-c3ccccc3)nc2c2nc(-c3ccccc3)ccc12)c1cc(-c2ccccn2)nc(-c2ccccn2)c1 ; C(#Cc1cc2ccc(-c3ccccn3)nc2c2nc(-c3ccccc3)ccc12)c1cc(-c2ccccn2)nc(-c2ccccn2)c1]

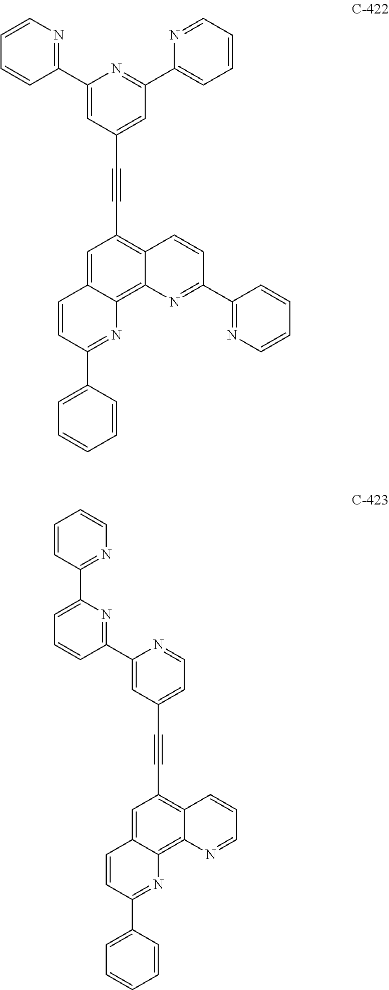 [SMILES: C(#Cc1cc2ccc(-c3ccccc3)nc2c2nc(-c3ccccn3)ccc12)c1cc(-c2ccccn2)nc(-c2ccccn2)c1 ; C(#Cc1cc2ccc(-c3ccccc3)nc2c2ncccc12)c1ccnc(-c2cccc(-c3ccccn3)n2)c1]

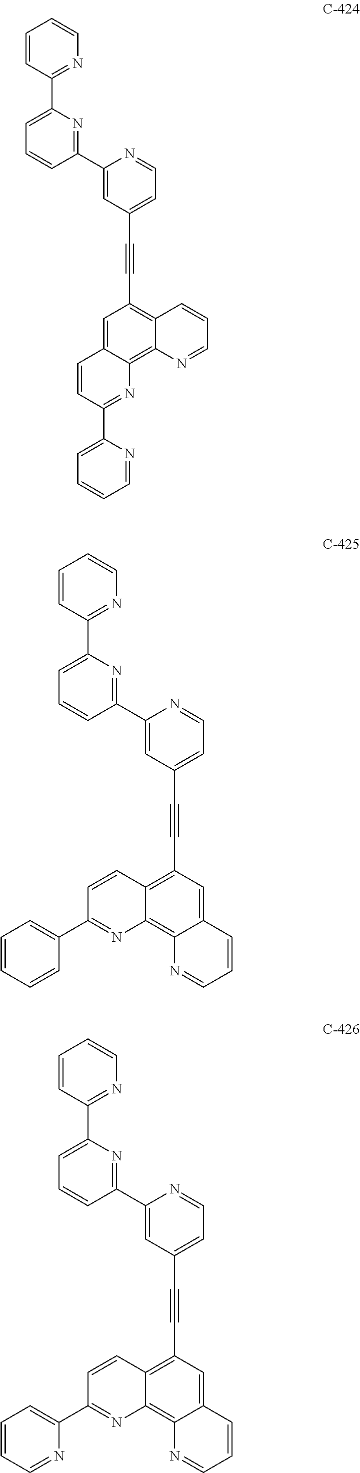 [SMILES: C(#Cc1cc2ccc(-c3ccccn3)nc2c2ncccc12)c1ccnc(-c2cccc(-c3ccccn3)n2)c1 ; C(#Cc1cc2cccnc2c2nc(-c3ccccc3)ccc12)c1ccnc(-c2cccc(-c3ccccn3)n2)c1 ; C(#Cc1cc2cccnc2c2nc(-c3ccccn3)ccc12)c1ccnc(-c2cccc(-c3ccccn3)n2)c1]

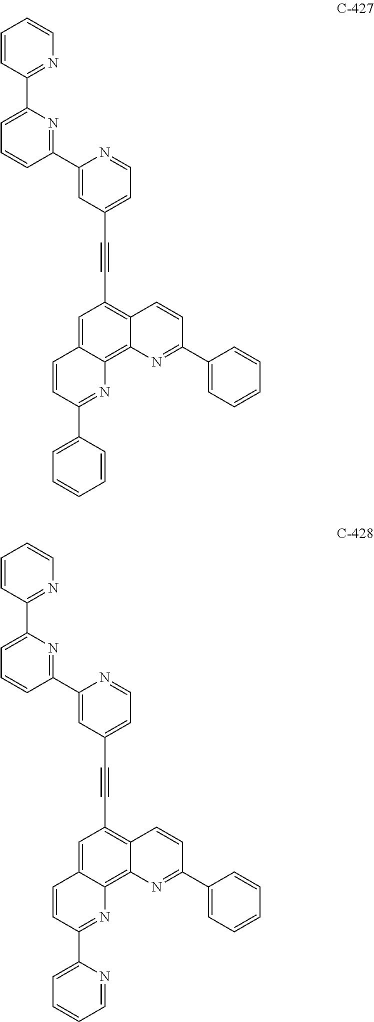 [SMILES: C(#Cc1cc2ccc(-c3ccccc3)nc2c2nc(-c3ccccc3)ccc12)c1ccnc(-c2cccc(-c3ccccn3)n2)c1 ; C(#Cc1cc2ccc(-c3ccccn3)nc2c2nc(-c3ccccc3)ccc12)c1ccnc(-c2cccc(-c3ccccn3)n2)c1]

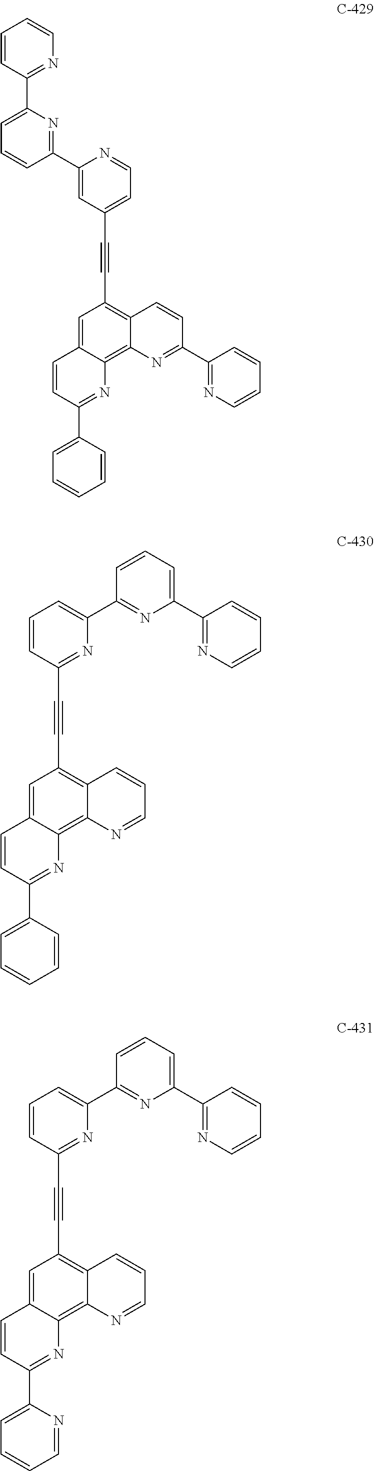 [SMILES: C(#Cc1cc2ccc(-c3ccccc3)nc2c2nc(-c3ccccn3)ccc12)c1ccnc(-c2cccc(-c3ccccn3)n2)c1 ; C(#Cc1cc2ccc(-c3ccccc3)nc2c2ncccc12)c1cccc(-c2cccc(-c3ccccn3)n2)n1 ; C(#Cc1cc2ccc(-c3ccccn3)nc2c2ncccc12)c1cccc(-c2cccc(-c3ccccn3)n2)n1]

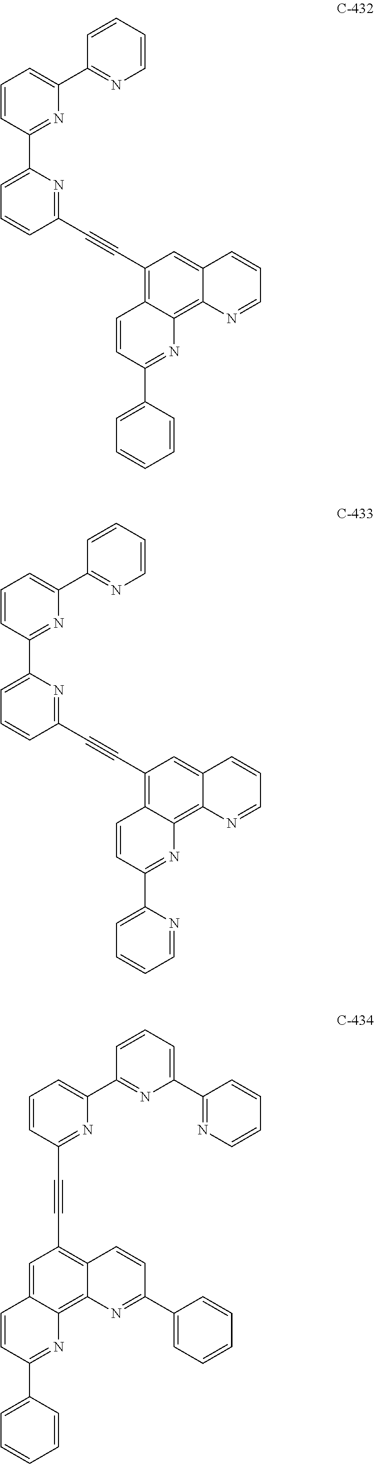 [SMILES: C(#Cc1cc2cccnc2c2nc(-c3ccccc3)ccc12)c1cccc(-c2cccc(-c3ccccn3)n2)n1 ; C(#Cc1cc2cccnc2c2nc(-c3ccccn3)ccc12)c1cccc(-c2cccc(-c3ccccn3)n2)n1 ; C(#Cc1cc2ccc(-c3ccccc3)nc2c2nc(-c3ccccc3)ccc12)c1cccc(-c2cccc(-c3ccccn3)n2)n1]

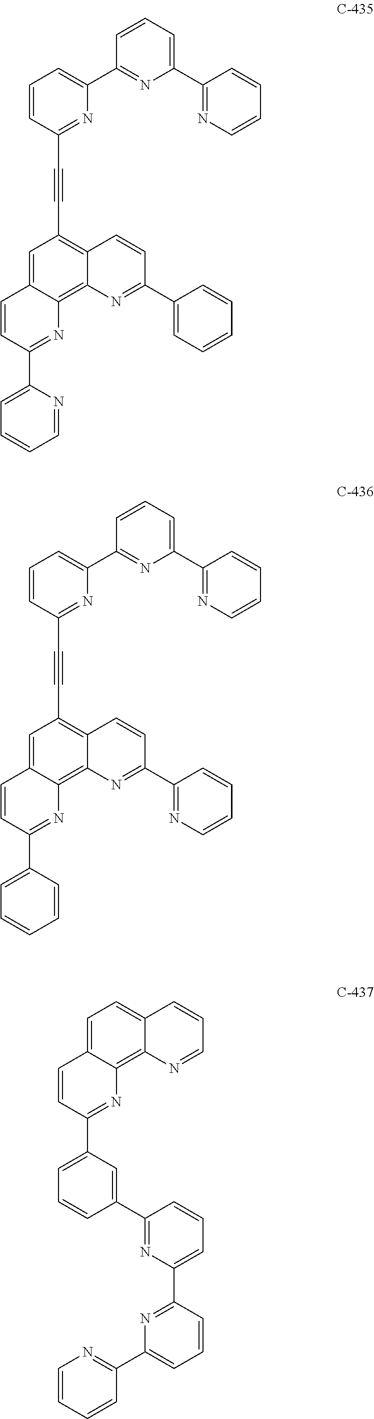 [SMILES: C(#Cc1cc2ccc(-c3ccccn3)nc2c2nc(-c3ccccc3)ccc12)c1cccc(-c2cccc(-c3ccccn3)n2)n1 ; C(#Cc1cc2ccc(-c3ccccc3)nc2c2nc(-c3ccccn3)ccc12)c1cccc(-c2cccc(-c3ccccn3)n2)n1 ; c1ccc(-c2cccc(-c3cccc(-c4cccc(-c5ccc6ccc7cccnc7c6n5)c4)n3)n2)nc1]

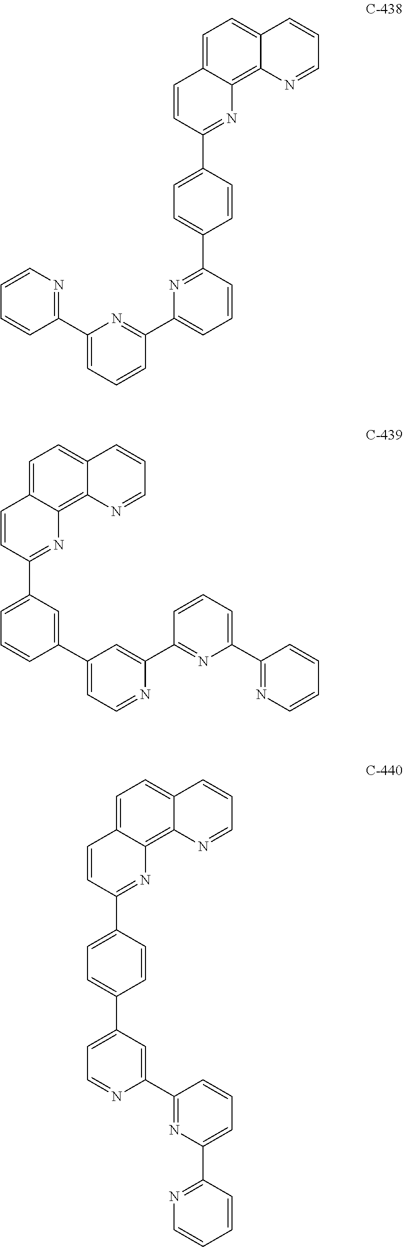 [SMILES: c1ccc(-c2cccc(-c3cccc(-c4ccc(-c5ccc6ccc7cccnc7c6n5)cc4)n3)n2)nc1 ; c1ccc(-c2cccc(-c3cc(-c4cccc(-c5ccc6ccc7cccnc7c6n5)c4)ccn3)n2)nc1 ; c1ccc(-c2cccc(-c3cc(-c4ccc(-c5ccc6ccc7cccnc7c6n5)cc4)ccn3)n2)nc1]

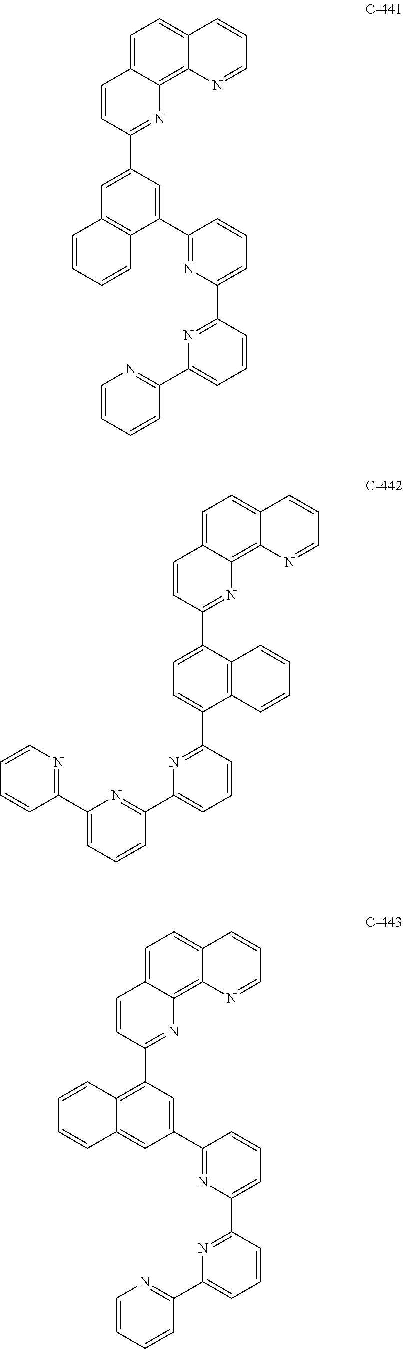 [SMILES: c1ccc(-c2cccc(-c3cccc(-c4cc(-c5ccc6ccc7cccnc7c6n5)cc5ccccc45)n3)n2)nc1 ; c1ccc(-c2cccc(-c3cccc(-c4ccc(-c5ccc6ccc7cccnc7c6n5)c5ccccc45)n3)n2)nc1 ; c1ccc(-c2cccc(-c3cccc(-c4cc(-c5ccc6ccc7cccnc7c6n5)c5ccccc5c4)n3)n2)nc1]

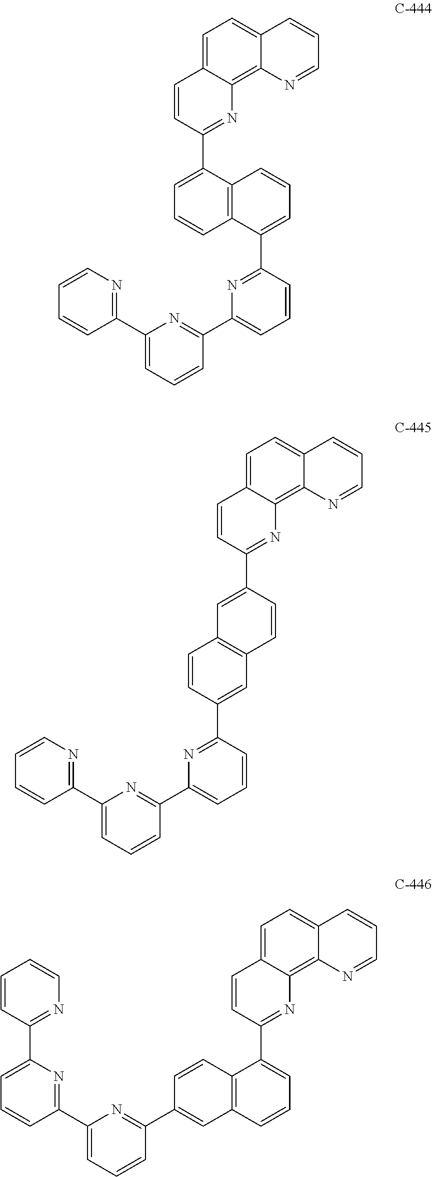 [SMILES: c1ccc(-c2cccc(-c3cccc(-c4cccc5c(-c6ccc7ccc8cccnc8c7n6)cccc45)n3)n2)nc1 ; c1ccc(-c2cccc(-c3cccc(-c4ccc5cc(-c6ccc7ccc8cccnc8c7n6)ccc5c4)n3)n2)nc1 ; c1ccc(-c2cccc(-c3cccc(-c4ccc5c(-c6ccc7ccc8cccnc8c7n6)cccc5c4)n3)n2)nc1]

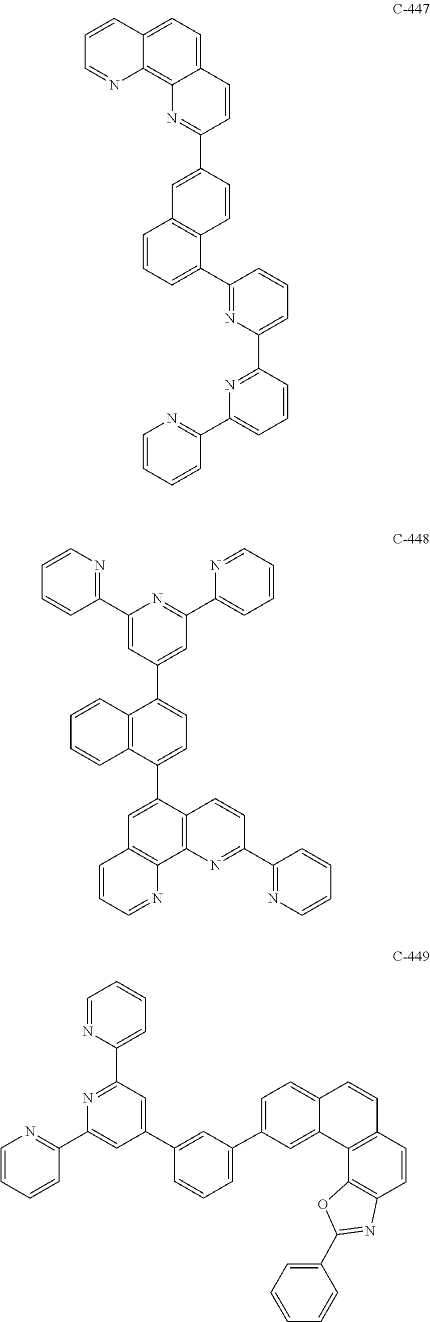

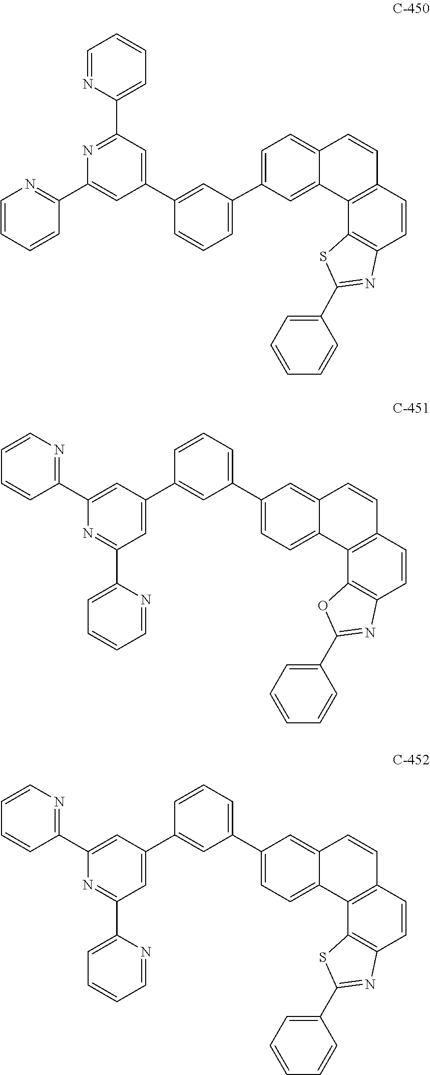

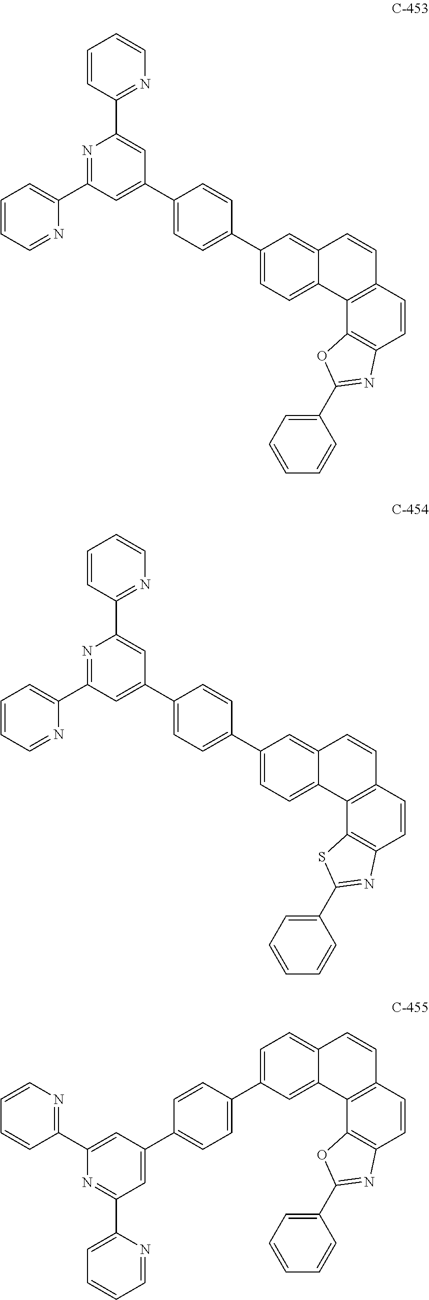 [SMILES: c1ccc(-c2nc3ccc4ccc5cc(-c6ccc(-c7cc(-c8ccccn8)nc(-c8ccccn8)c7)cc6)ccc5c4c3o2)cc1 ; c1ccc(-c2nc3ccc4ccc5cc(-c6ccc(-c7cc(-c8ccccn8)nc(-c8ccccn8)c7)cc6)ccc5c4c3s2)cc1 ; c1ccc(-c2nc3ccc4ccc5ccc(-c6ccc(-c7cc(-c8ccccn8)nc(-c8ccccn8)c7)cc6)cc5c4c3o2)cc1]

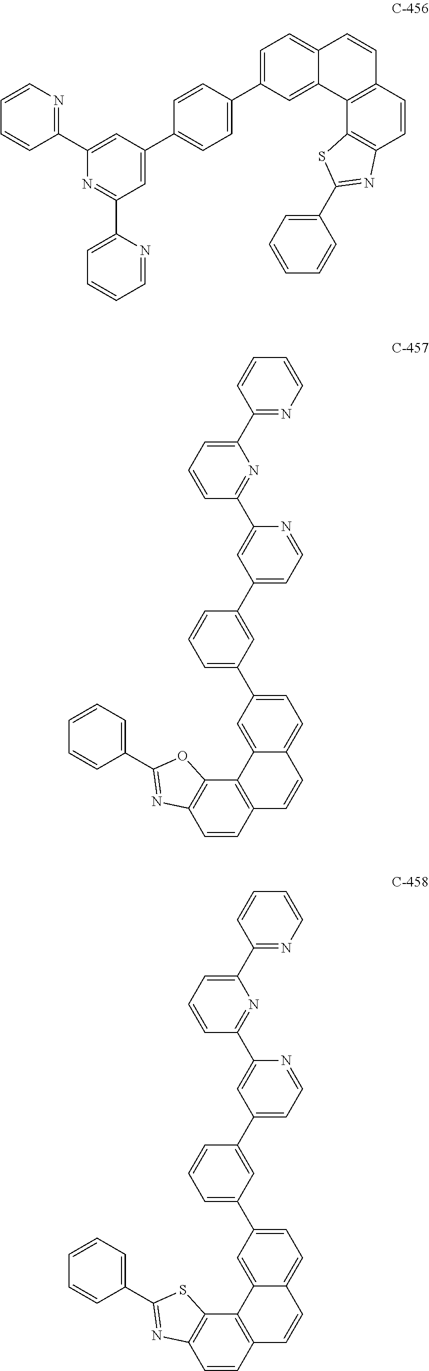 [SMILES: c1ccc(-c2nc3ccc4ccc5ccc(-c6ccc(-c7cc(-c8ccccn8)nc(-c8ccccn8)c7)cc6)cc5c4c3s2)cc1 ; c1ccc(-c2nc3ccc4ccc5ccc(-c6cccc(-c7ccnc(-c8cccc(-c9ccccn9)n8)c7)c6)cc5c4c3o2)cc1 ; c1ccc(-c2nc3ccc4ccc5ccc(-c6cccc(-c7ccnc(-c8cccc(-c9ccccn9)n8)c7)c6)cc5c4c3s2)cc1]

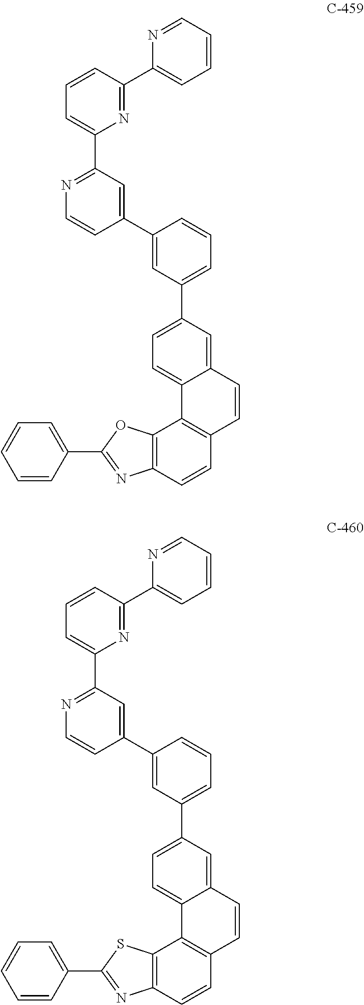 [SMILES: c1ccc(-c2nc3ccc4ccc5cc(-c6cccc(-c7ccnc(-c8cccc(-c9ccccn9)n8)c7)c6)ccc5c4c3o2)cc1 ; c1ccc(-c2nc3ccc4ccc5cc(-c6cccc(-c7ccnc(-c8cccc(-c9ccccn9)n8)c7)c6)ccc5c4c3s2)cc1]

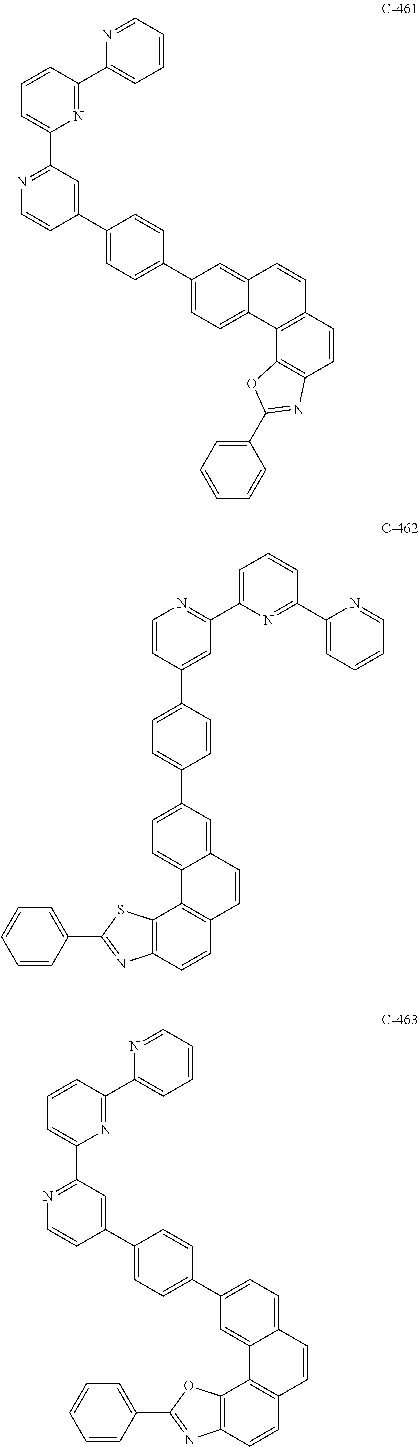 [SMILES: c1ccc(-c2nc3ccc4ccc5cc(-c6ccc(-c7ccnc(-c8cccc(-c9ccccn9)n8)c7)cc6)ccc5c4c3o2)cc1 ; c1ccc(-c2nc3ccc4ccc5cc(-c6ccc(-c7ccnc(-c8cccc(-c9ccccn9)n8)c7)cc6)ccc5c4c3s2)cc1 ; c1ccc(-c2nc3ccc4ccc5ccc(-c6ccc(-c7ccnc(-c8cccc(-c9ccccn9)n8)c7)cc6)cc5c4c3o2)cc1]

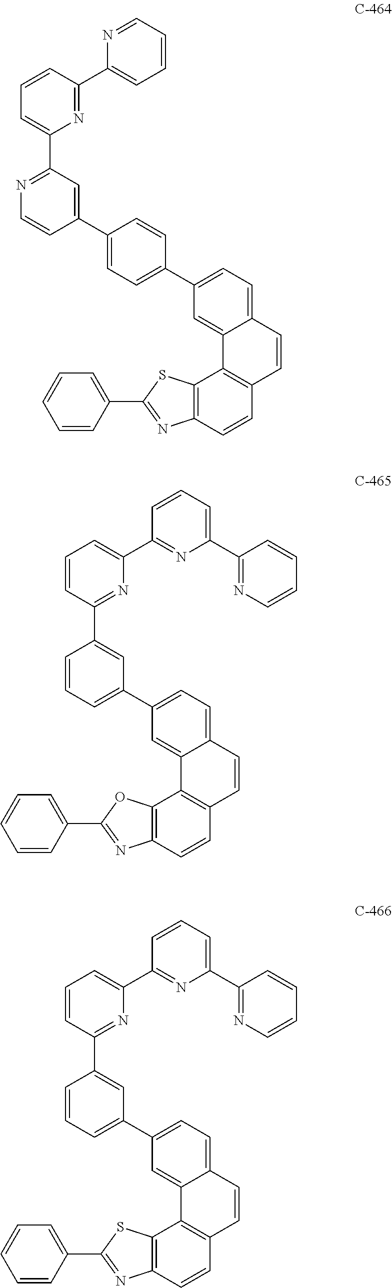 [SMILES: c1ccc(-c2nc3ccc4ccc5ccc(-c6ccc(-c7ccnc(-c8cccc(-c9ccccn9)n8)c7)cc6)cc5c4c3s2)cc1 ; c1ccc(-c2nc3ccc4ccc5ccc(-c6cccc(-c7cccc(-c8cccc(-c9ccccn9)n8)n7)c6)cc5c4c3o2)cc1 ; c1ccc(-c2nc3ccc4ccc5ccc(-c6cccc(-c7cccc(-c8cccc(-c9ccccn9)n8)n7)c6)cc5c4c3s2)cc1]

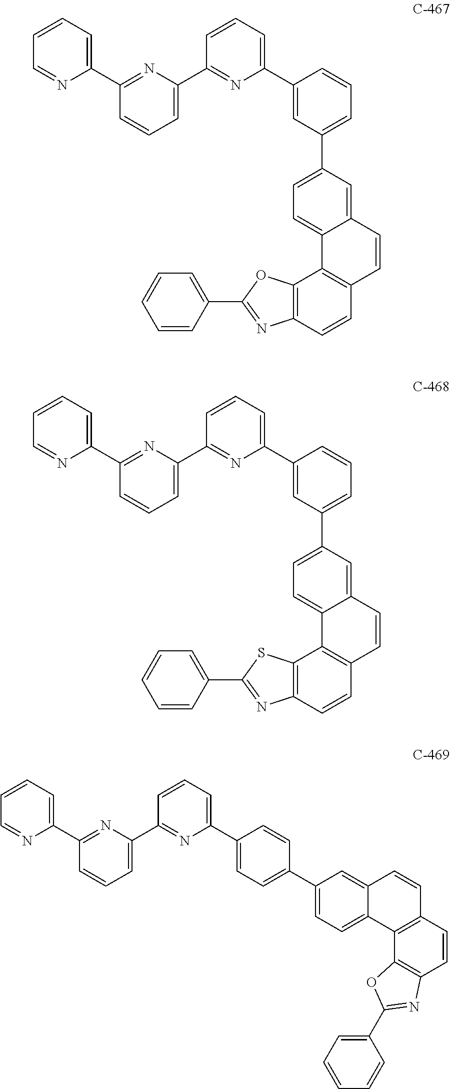 [SMILES: c1ccc(-c2nc3ccc4ccc5cc(-c6cccc(-c7cccc(-c8cccc(-c9ccccn9)n8)n7)c6)ccc5c4c3o2)cc1 ; c1ccc(-c2nc3ccc4ccc5cc(-c6cccc(-c7cccc(-c8cccc(-c9ccccn9)n8)n7)c6)ccc5c4c3s2)cc1 ; c1ccc(-c2nc3ccc4ccc5cc(-c6ccc(-c7cccc(-c8cccc(-c9ccccn9)n8)n7)cc6)ccc5c4c3o2)cc1]

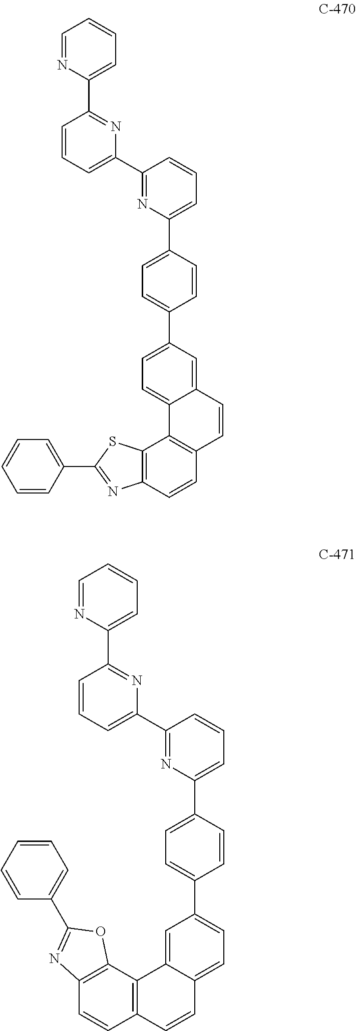 [SMILES: c1ccc(-c2nc3ccc4ccc5cc(-c6ccc(-c7cccc(-c8cccc(-c9ccccn9)n8)n7)cc6)ccc5c4c3s2)cc1 ; c1ccc(-c2nc3ccc4ccc5ccc(-c6ccc(-c7cccc(-c8cccc(-c9ccccn9)n8)n7)cc6)cc5c4c3o2)cc1]

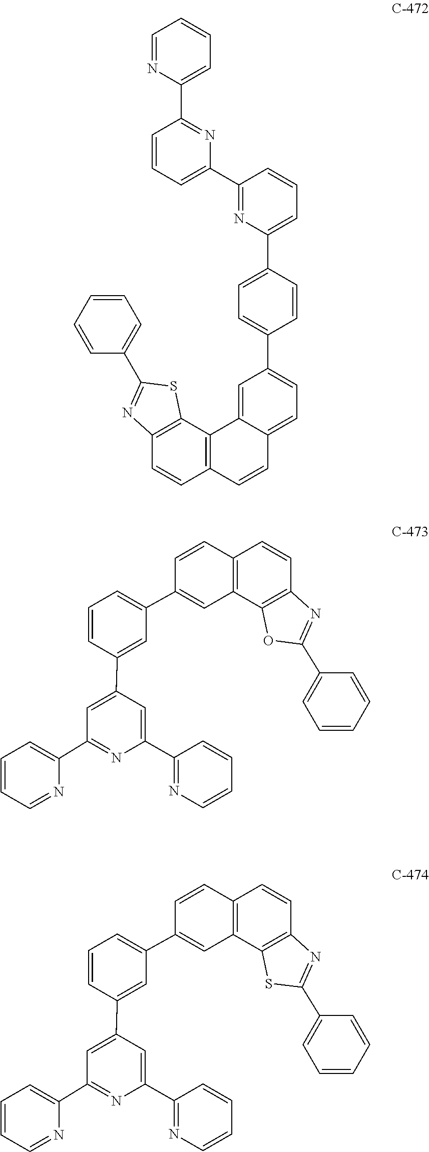 [SMILES: c1ccc(-c2nc3ccc4ccc5ccc(-c6ccc(-c7cccc(-c8cccc(-c9ccccn9)n8)n7)cc6)cc5c4c3s2)cc1 ; c1ccc(-c2nc3ccc4ccc(-c5cccc(-c6cc(-c7ccccn7)nc(-c7ccccn7)c6)c5)cc4c3o2)cc1 ; c1ccc(-c2nc3ccc4ccc(-c5cccc(-c6cc(-c7ccccn7)nc(-c7ccccn7)c6)c5)cc4c3s2)cc1]

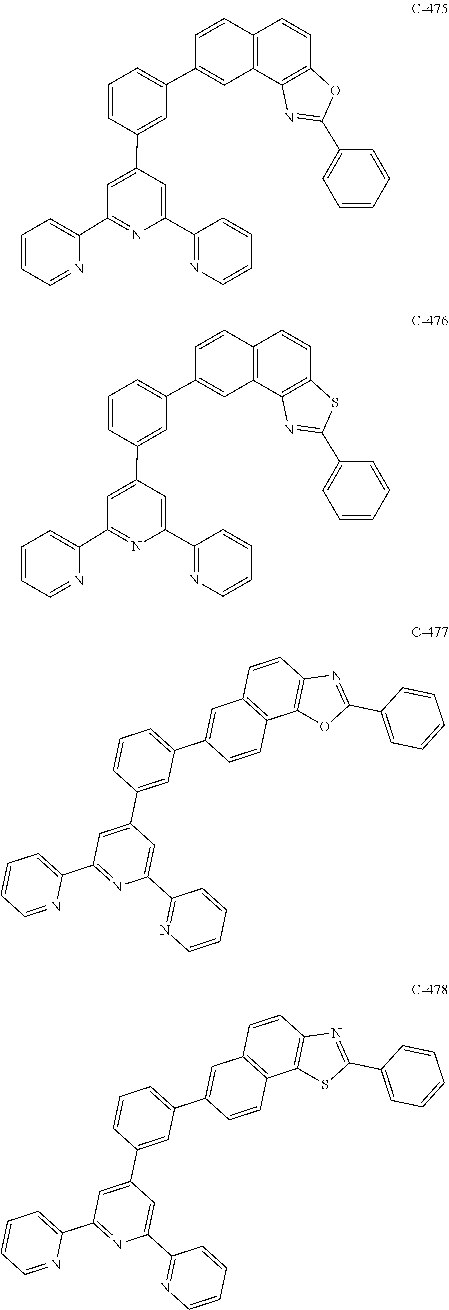

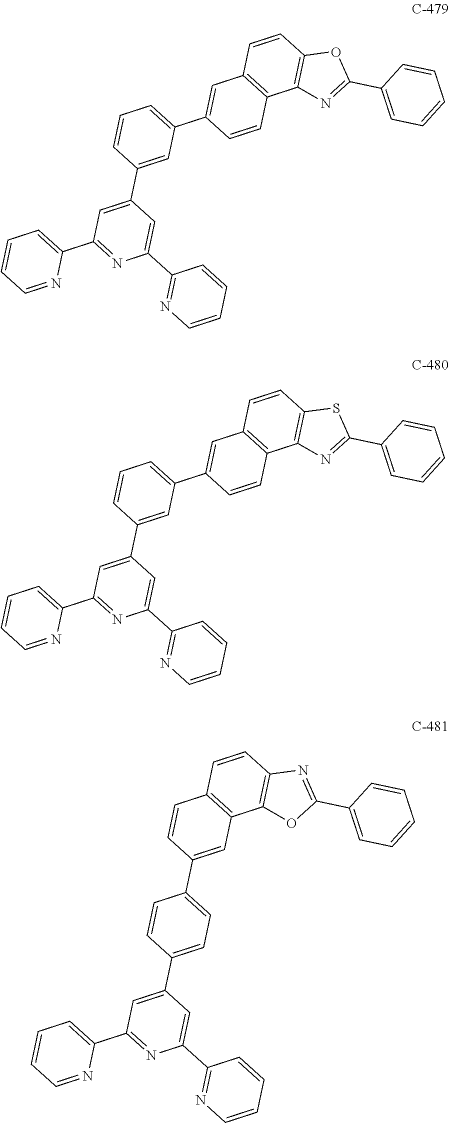 [SMILES: c1ccc(-c2nc3c(ccc4cc(-c5cccc(-c6cc(-c7ccccn7)nc(-c7ccccn7)c6)c5)ccc43)o2)cc1 ; c1ccc(-c2nc3c(ccc4cc(-c5cccc(-c6cc(-c7ccccn7)nc(-c7ccccn7)c6)c5)ccc43)s2)cc1 ; c1ccc(-c2nc3ccc4ccc(-c5ccc(-c6cc(-c7ccccn7)nc(-c7ccccn7)c6)cc5)cc4c3o2)cc1]

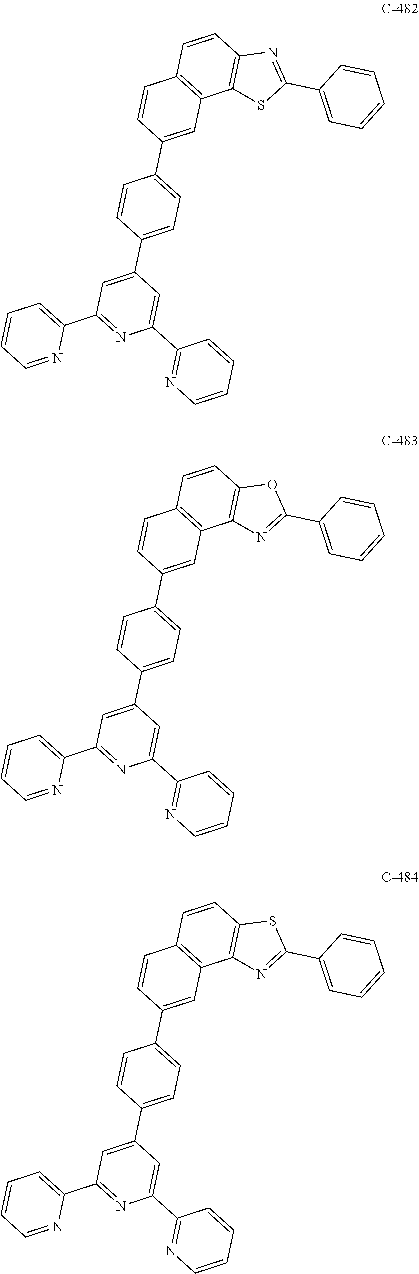 [SMILES: c1ccc(-c2nc3ccc4ccc(-c5ccc(-c6cc(-c7ccccn7)nc(-c7ccccn7)c6)cc5)cc4c3s2)cc1 ; c1ccc(-c2nc3c(ccc4ccc(-c5ccc(-c6cc(-c7ccccn7)nc(-c7ccccn7)c6)cc5)cc43)o2)cc1 ; c1ccc(-c2nc3c(ccc4ccc(-c5ccc(-c6cc(-c7ccccn7)nc(-c7ccccn7)c6)cc5)cc43)s2)cc1]

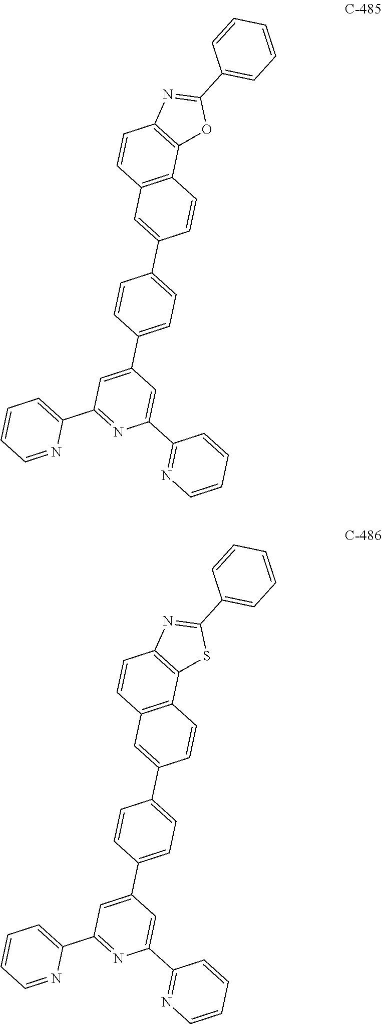 [SMILES: c1ccc(-c2nc3ccc4cc(-c5ccc(-c6cc(-c7ccccn7)nc(-c7ccccn7)c6)cc5)ccc4c3o2)cc1 ; c1ccc(-c2nc3ccc4cc(-c5ccc(-c6cc(-c7ccccn7)nc(-c7ccccn7)c6)cc5)ccc4c3s2)cc1]

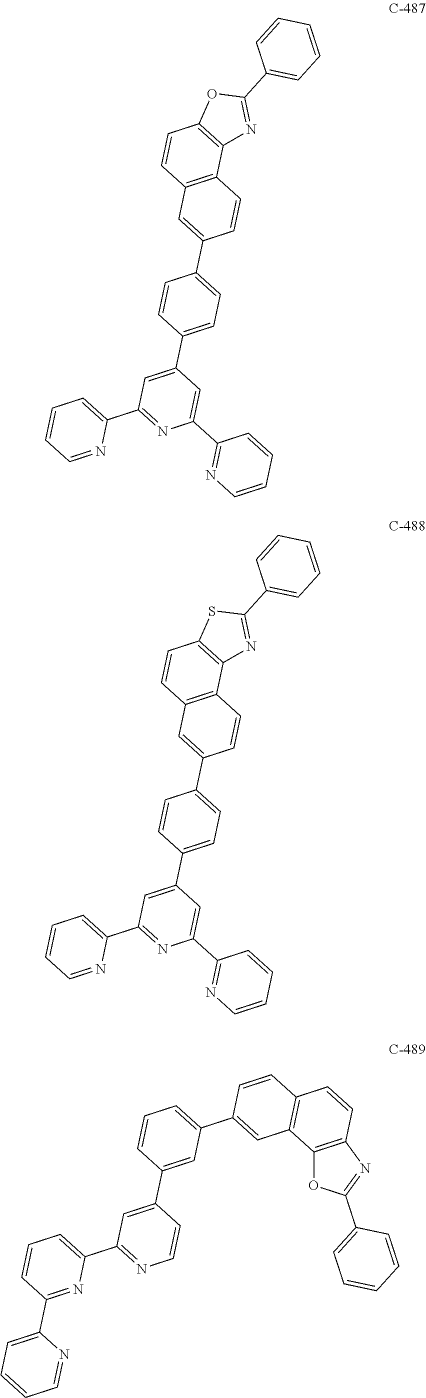 [SMILES: c1ccc(-c2nc3c(ccc4cc(-c5ccc(-c6cc(-c7ccccn7)nc(-c7ccccn7)c6)cc5)ccc43)o2)cc1 ; c1ccc(-c2nc3c(ccc4cc(-c5ccc(-c6cc(-c7ccccn7)nc(-c7ccccn7)c6)cc5)ccc43)s2)cc1 ; c1ccc(-c2nc3ccc4ccc(-c5cccc(-c6ccnc(-c7cccc(-c8ccccn8)n7)c6)c5)cc4c3o2)cc1]

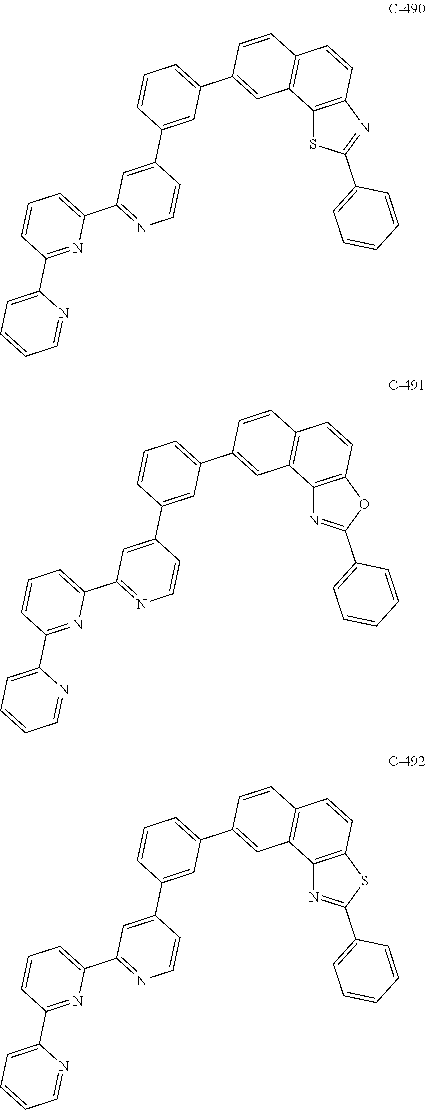 [SMILES: c1ccc(-c2nc3ccc4ccc(-c5cccc(-c6ccnc(-c7cccc(-c8ccccn8)n7)c6)c5)cc4c3s2)cc1 ; c1ccc(-c2nc3c(ccc4ccc(-c5cccc(-c6ccnc(-c7cccc(-c8ccccn8)n7)c6)c5)cc43)o2)cc1 ; c1ccc(-c2nc3c(ccc4ccc(-c5cccc(-c6ccnc(-c7cccc(-c8ccccn8)n7)c6)c5)cc43)s2)cc1]

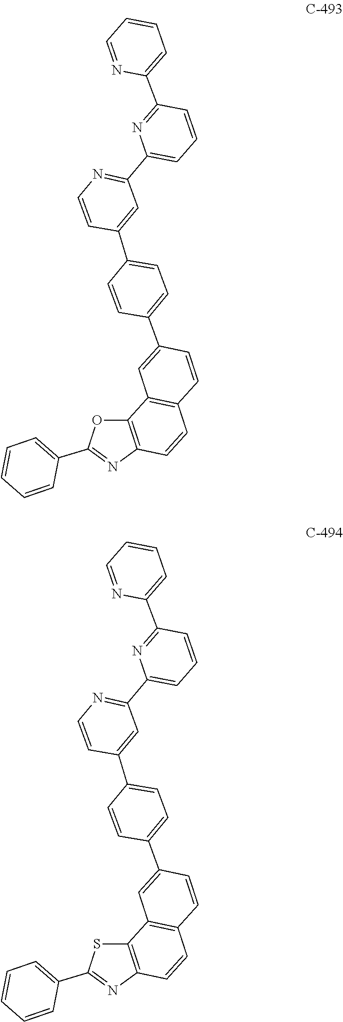 [SMILES: c1ccc(-c2nc3ccc4ccc(-c5ccc(-c6ccnc(-c7cccc(-c8ccccn8)n7)c6)cc5)cc4c3o2)cc1 ; c1ccc(-c2nc3ccc4ccc(-c5ccc(-c6ccnc(-c7cccc(-c8ccccn8)n7)c6)cc5)cc4c3s2)cc1]

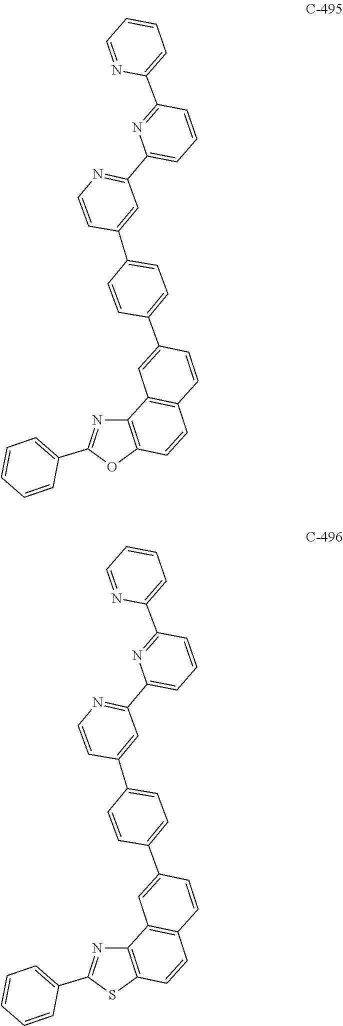 [SMILES: c1ccc(-c2nc3c(ccc4ccc(-c5ccc(-c6ccnc(-c7cccc(-c8ccccn8)n7)c6)cc5)cc43)o2)cc1 ; c1ccc(-c2nc3c(ccc4ccc(-c5ccc(-c6ccnc(-c7cccc(-c8ccccn8)n7)c6)cc5)cc43)s2)cc1]

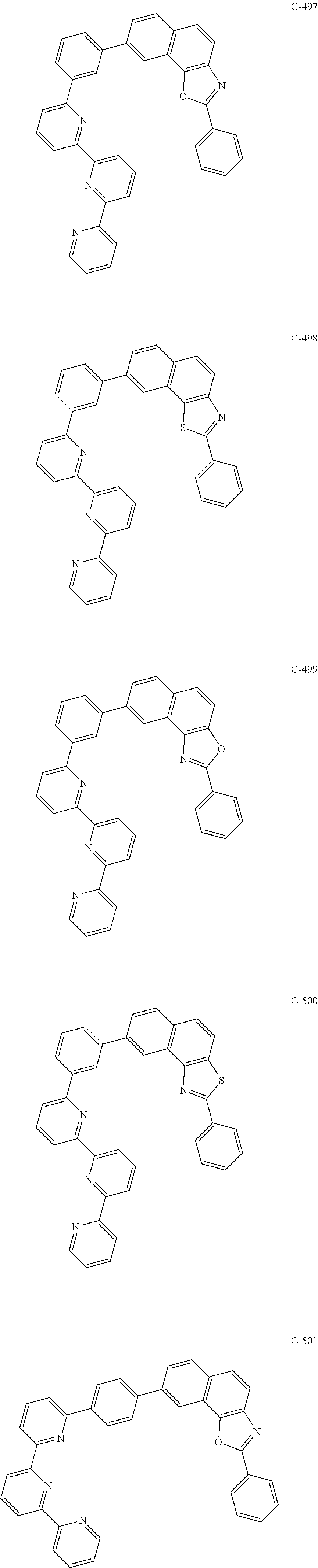 [SMILES: c1ccc(-c2nc3ccc4ccc(-c5cccc(-c6cccc(-c7cccc(-c8ccccn8)n7)n6)c5)cc4c3o2)cc1 ; c1ccc(-c2nc3ccc4ccc(-c5cccc(-c6cccc(-c7cccc(-c8ccccn8)n7)n6)c5)cc4c3s2)cc1 ; c1ccc(-c2nc3c(ccc4ccc(-c5cccc(-c6cccc(-c7cccc(-c8ccccn8)n7)n6)c5)cc43)o2)cc1 ; c1ccc(-c2nc3c(ccc4ccc(-c5cccc(-c6cccc(-c7cccc(-c8ccccn8)n7)n6)c5)cc43)s2)cc1 ; c1ccc(-c2nc3ccc4ccc(-c5ccc(-c6cccc(-c7cccc(-c8ccccn8)n7)n6)cc5)cc4c3o2)cc1]

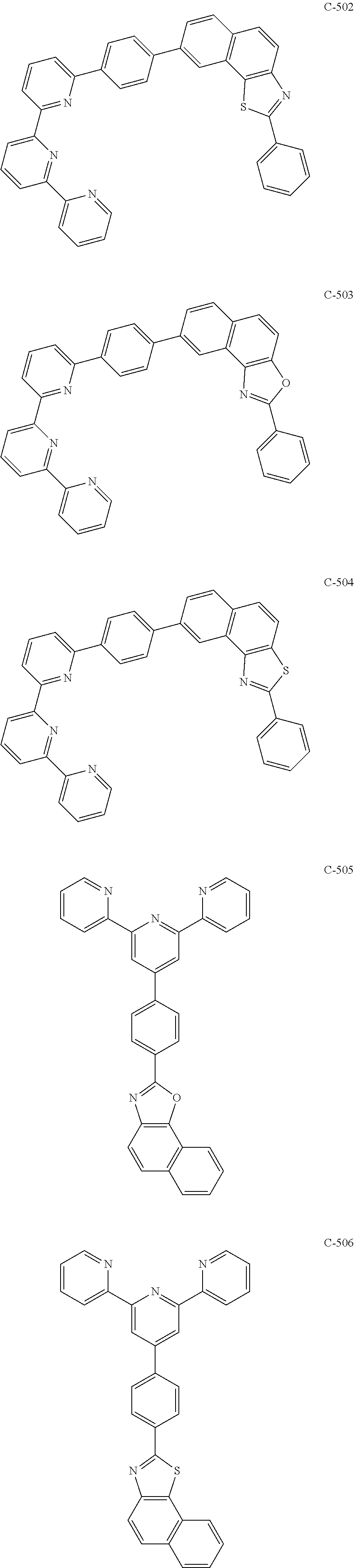 [SMILES: c1ccc(-c2nc3ccc4ccc(-c5ccc(-c6cccc(-c7cccc(-c8ccccn8)n7)n6)cc5)cc4c3s2)cc1 ; c1ccc(-c2nc3c(ccc4ccc(-c5ccc(-c6cccc(-c7cccc(-c8ccccn8)n7)n6)cc5)cc43)o2)cc1 ; c1ccc(-c2nc3c(ccc4ccc(-c5ccc(-c6cccc(-c7cccc(-c8ccccn8)n7)n6)cc5)cc43)s2)cc1 ; c1ccc(-c2cc(-c3ccc(-c4nc5ccc6ccccc6c5o4)cc3)cc(-c3ccccn3)n2)nc1 ; c1ccc(-c2cc(-c3ccc(-c4nc5ccc6ccccc6c5s4)cc3)cc(-c3ccccn3)n2)nc1]

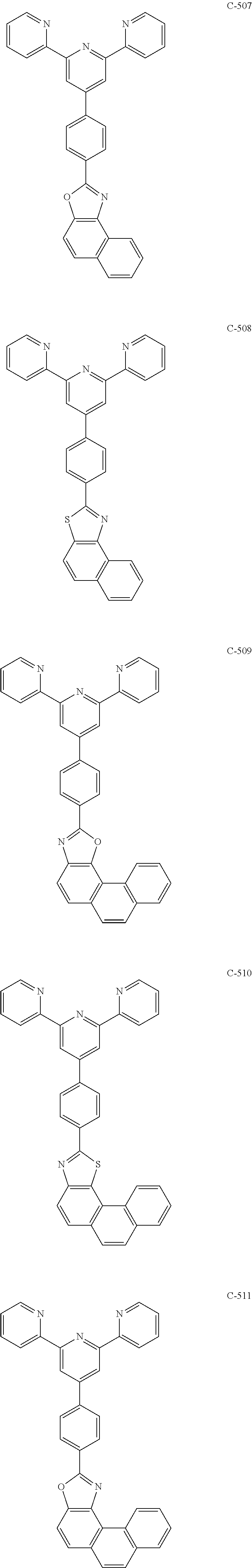 [SMILES: c1ccc(-c2cc(-c3ccc(-c4nc5c(ccc6ccccc65)o4)cc3)cc(-c3ccccn3)n2)nc1 ; c1ccc(-c2cc(-c3ccc(-c4nc5c(ccc6ccccc65)s4)cc3)cc(-c3ccccn3)n2)nc1 ; c1ccc(-c2cc(-c3ccc(-c4nc5ccc6ccc7ccccc7c6c5o4)cc3)cc(-c3ccccn3)n2)nc1 ; c1ccc(-c2cc(-c3ccc(-c4nc5ccc6ccc7ccccc7c6c5s4)cc3)cc(-c3ccccn3)n2)nc1 ; c1ccc(-c2cc(-c3ccc(-c4nc5c(ccc6ccc7ccccc7c65)o4)cc3)cc(-c3ccccn3)n2)nc1]

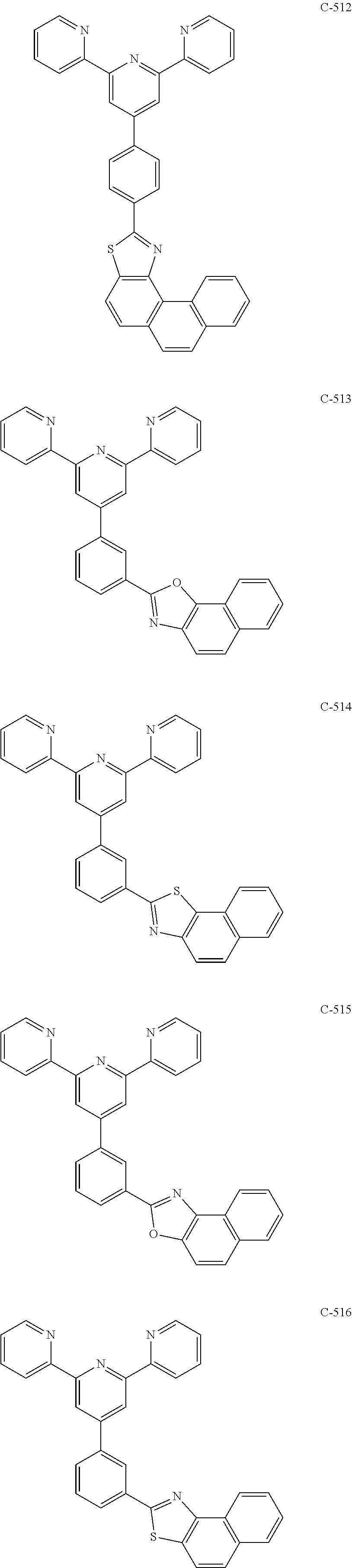 [SMILES: c1ccc(-c2cc(-c3ccc(-c4nc5c(ccc6ccc7ccccc7c65)s4)cc3)cc(-c3ccccn3)n2)nc1 ; c1ccc(-c2cc(-c3cccc(-c4nc5ccc6ccccc6c5o4)c3)cc(-c3ccccn3)n2)nc1 ; c1ccc(-c2cc(-c3cccc(-c4nc5ccc6ccccc6c5s4)c3)cc(-c3ccccn3)n2)nc1 ; c1ccc(-c2cc(-c3cccc(-c4nc5c(ccc6ccccc65)o4)c3)cc(-c3ccccn3)n2)nc1 ; c1ccc(-c2cc(-c3cccc(-c4nc5c(ccc6ccccc65)s4)c3)cc(-c3ccccn3)n2)nc1]

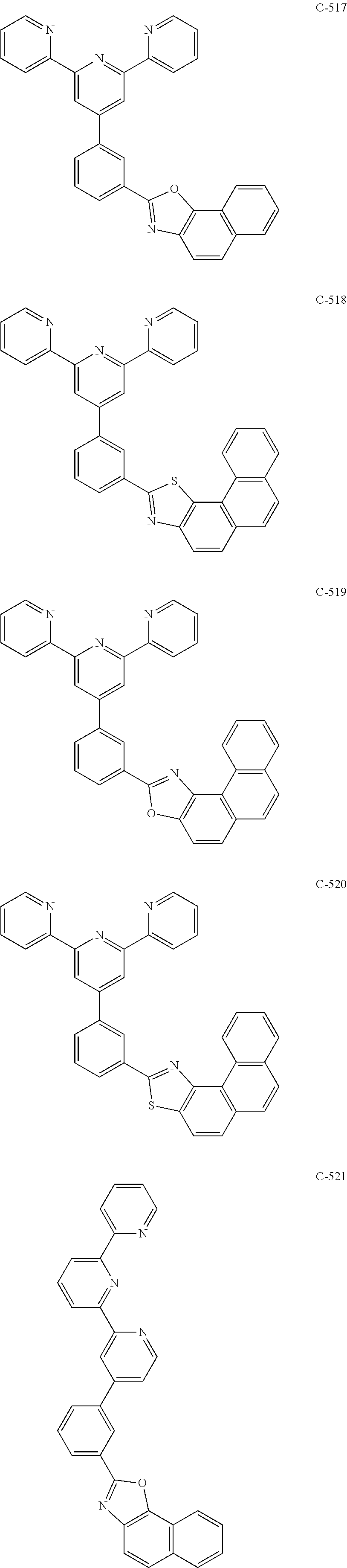 [SMILES: c1ccc(-c2cc(-c3cccc(-c4nc5ccc6ccccc6c5o4)c3)cc(-c3ccccn3)n2)nc1 ; c1ccc(-c2cc(-c3cccc(-c4nc5ccc6ccc7ccccc7c6c5s4)c3)cc(-c3ccccn3)n2)nc1 ; c1ccc(-c2cc(-c3cccc(-c4nc5c(ccc6ccc7ccccc7c65)o4)c3)cc(-c3ccccn3)n2)nc1 ; c1ccc(-c2cc(-c3cccc(-c4nc5c(ccc6ccc7ccccc7c65)s4)c3)cc(-c3ccccn3)n2)nc1 ; c1ccc(-c2cccc(-c3cc(-c4cccc(-c5nc6ccc7ccccc7c6o5)c4)ccn3)n2)nc1]

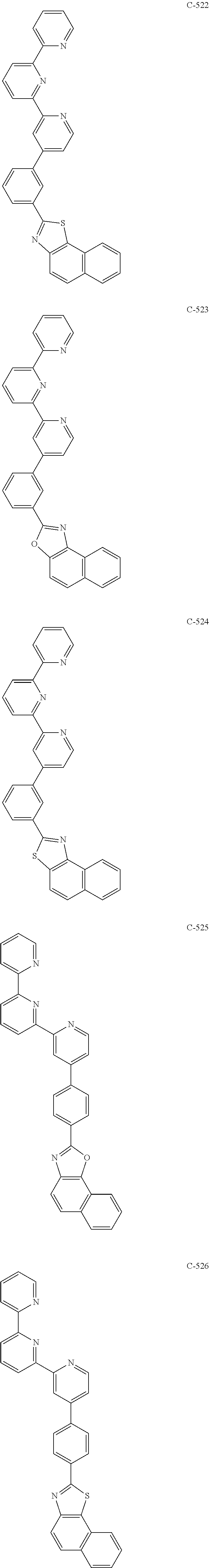 [SMILES: c1ccc(-c2cccc(-c3cc(-c4cccc(-c5nc6ccc7ccccc7c6s5)c4)ccn3)n2)nc1 ; c1ccc(-c2cccc(-c3cc(-c4cccc(-c5nc6c(ccc7ccccc76)o5)c4)ccn3)n2)nc1 ; c1ccc(-c2cccc(-c3cc(-c4cccc(-c5nc6c(ccc7ccccc76)s5)c4)ccn3)n2)nc1 ; c1ccc(-c2cccc(-c3cc(-c4ccc(-c5nc6ccc7ccccc7c6o5)cc4)ccn3)n2)nc1 ; c1ccc(-c2cccc(-c3cc(-c4ccc(-c5nc6ccc7ccccc7c6s5)cc4)ccn3)n2)nc1]

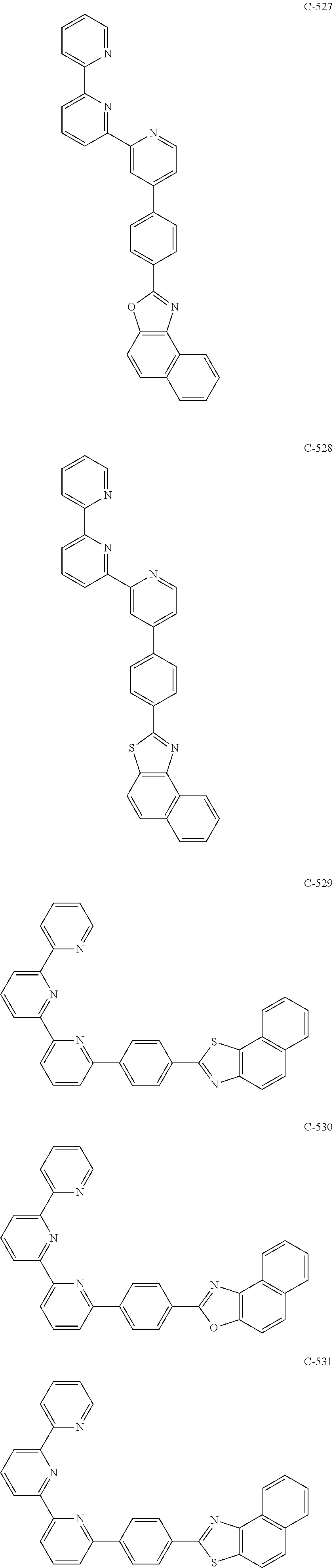 [SMILES: c1ccc(-c2cccc(-c3cc(-c4ccc(-c5nc6c(ccc7ccccc76)o5)cc4)ccn3)n2)nc1 ; c1ccc(-c2cccc(-c3cc(-c4ccc(-c5nc6c(ccc7ccccc76)s5)cc4)ccn3)n2)nc1 ; c1ccc(-c2cccc(-c3cccc(-c4ccc(-c5nc6ccc7ccccc7c6s5)cc4)n3)n2)nc1 ; c1ccc(-c2cccc(-c3cccc(-c4ccc(-c5nc6c(ccc7ccccc76)o5)cc4)n3)n2)nc1 ; c1ccc(-c2cccc(-c3cccc(-c4ccc(-c5nc6c(ccc7ccccc76)s5)cc4)n3)n2)nc1]

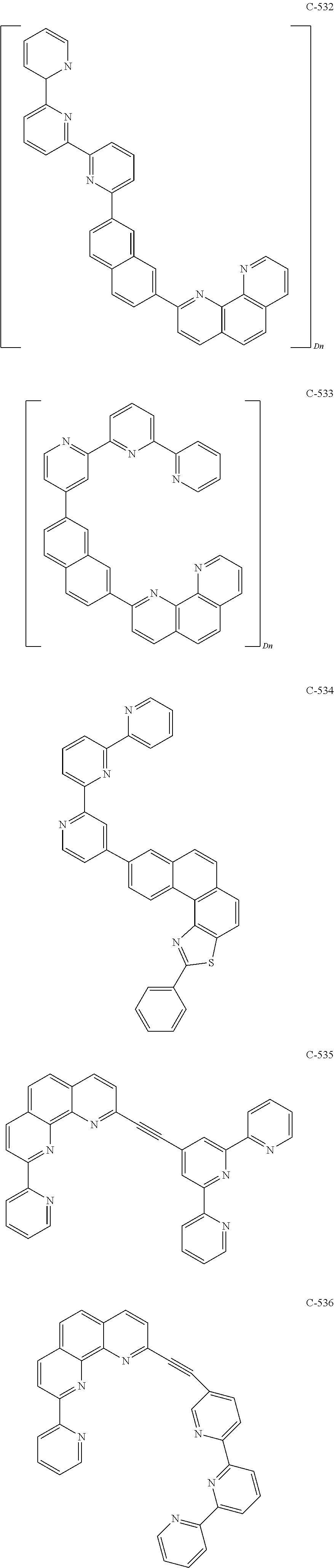

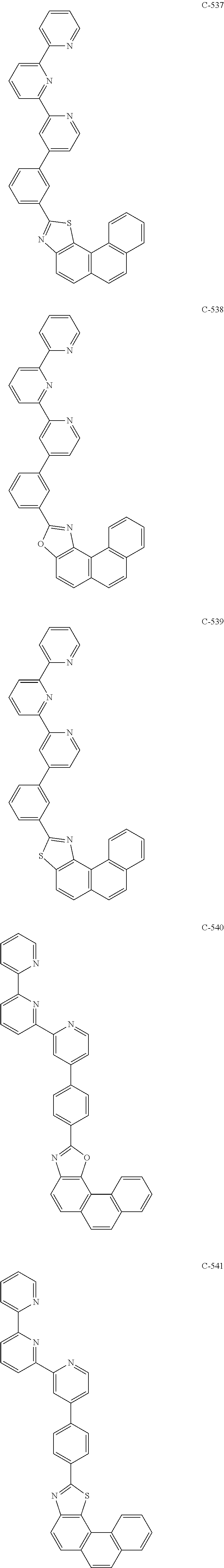 [SMILES: c1ccc(-c2cccc(-c3cc(-c4cccc(-c5nc6ccc7ccc8ccccc8c7c6s5)c4)ccn3)n2)nc1 ; c1ccc(-c2cccc(-c3cc(-c4cccc(-c5nc6c(ccc7ccc8ccccc8c76)o5)c4)ccn3)n2)nc1 ; c1ccc(-c2cccc(-c3cc(-c4cccc(-c5nc6c(ccc7ccc8ccccc8c76)s5)c4)ccn3)n2)nc1 ; c1ccc(-c2cccc(-c3cc(-c4ccc(-c5nc6ccc7ccc8ccccc8c7c6o5)cc4)ccn3)n2)nc1 ; c1ccc(-c2cccc(-c3cc(-c4ccc(-c5nc6ccc7ccc8ccccc8c7c6s5)cc4)ccn3)n2)nc1]

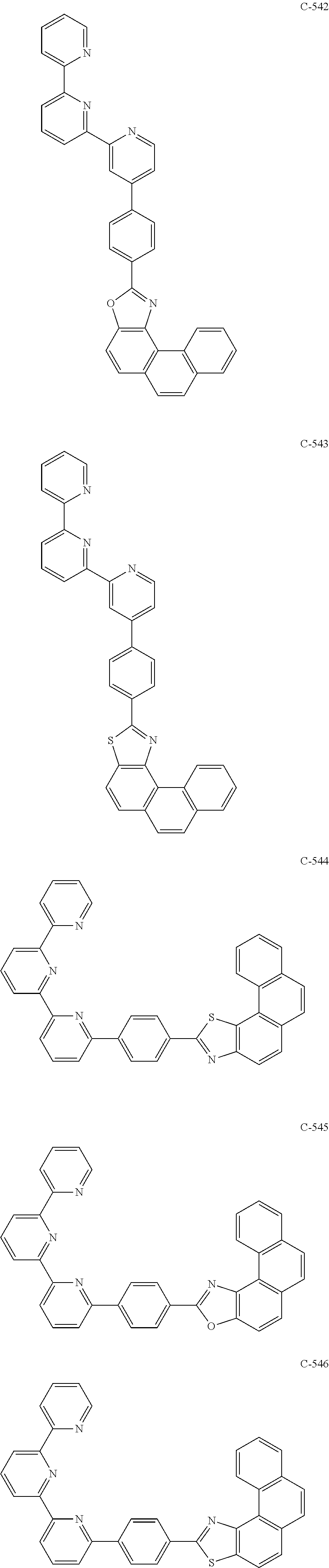 [SMILES: c1ccc(-c2cccc(-c3cc(-c4ccc(-c5nc6c(ccc7ccc8ccccc8c76)o5)cc4)ccn3)n2)nc1 ; c1ccc(-c2cccc(-c3cc(-c4ccc(-c5nc6c(ccc7ccc8ccccc8c76)s5)cc4)ccn3)n2)nc1 ; c1ccc(-c2cccc(-c3cccc(-c4ccc(-c5nc6ccc7ccc8ccccc8c7c6s5)cc4)n3)n2)nc1 ; c1ccc(-c2cccc(-c3cccc(-c4ccc(-c5nc6c(ccc7ccc8ccccc8c76)o5)cc4)n3)n2)nc1 ; c1ccc(-c2cccc(-c3cccc(-c4ccc(-c5nc6c(ccc7ccc8ccccc8c76)s5)cc4)n3)n2)nc1]

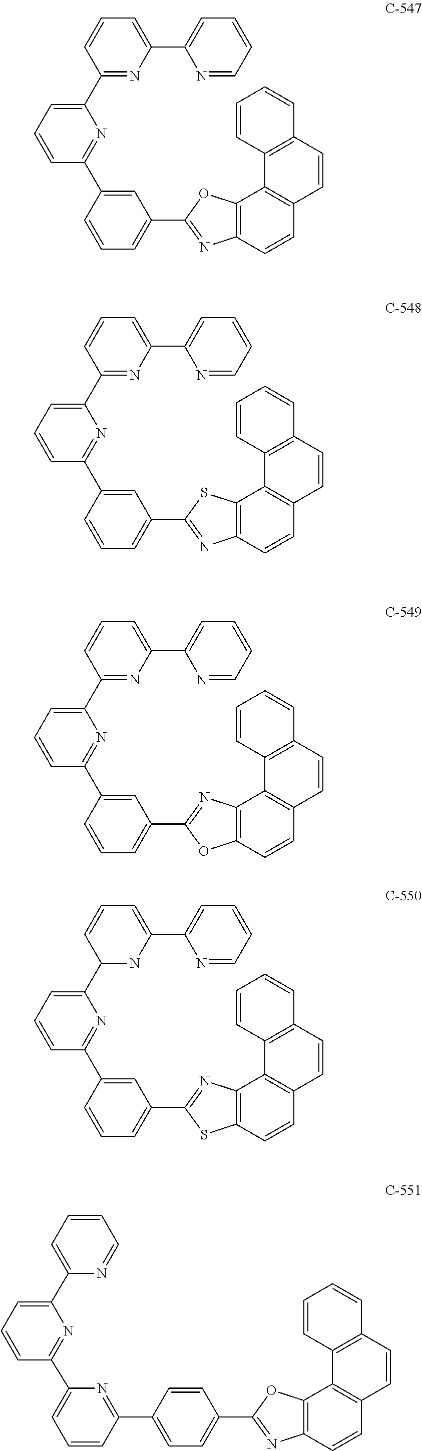

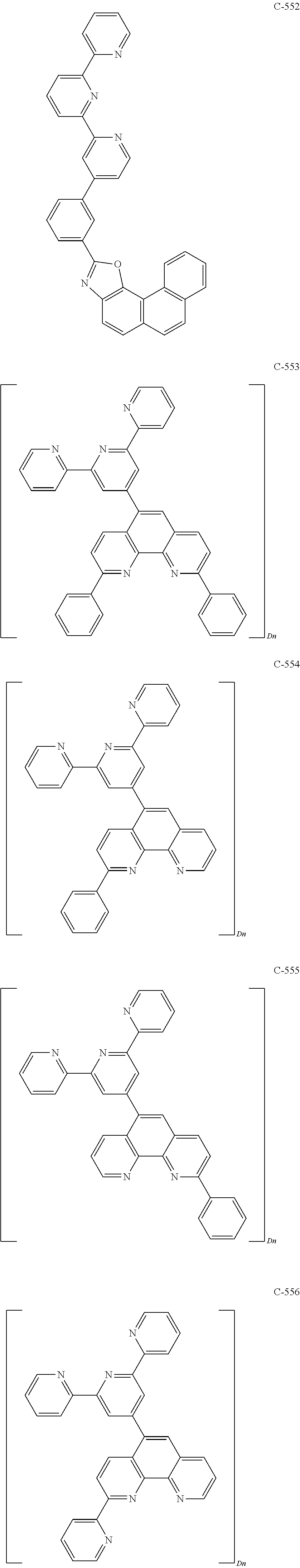

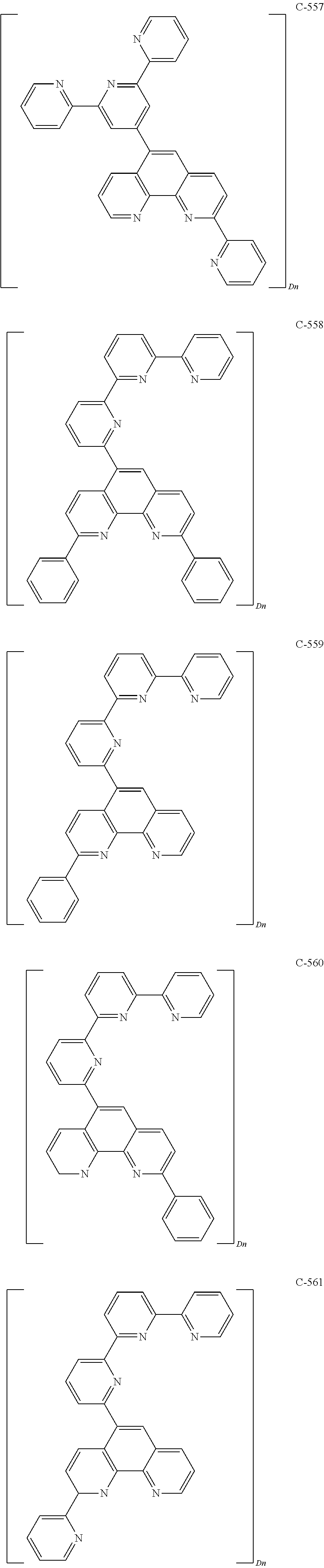

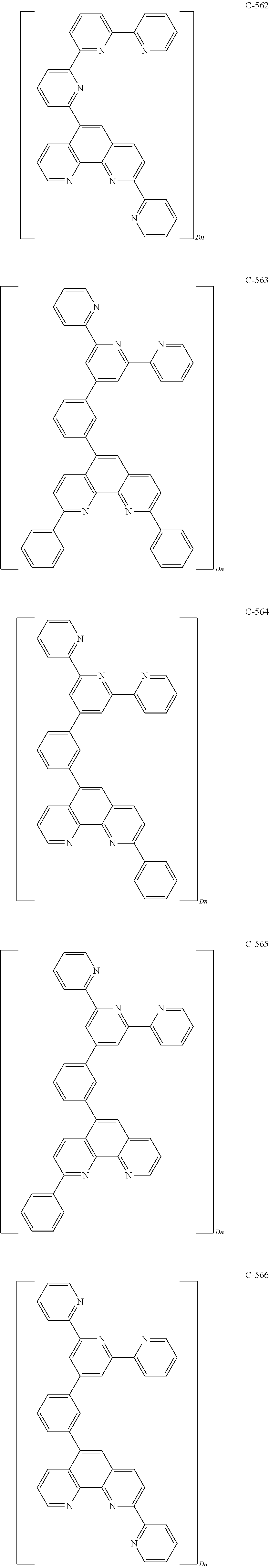

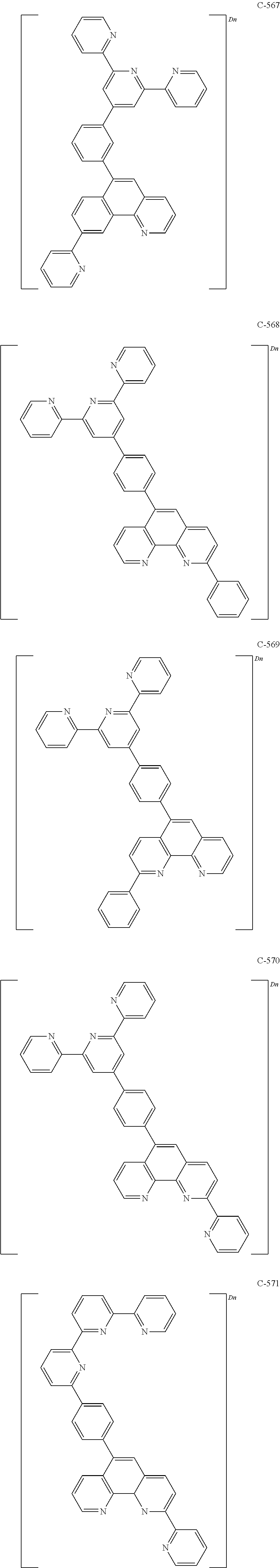

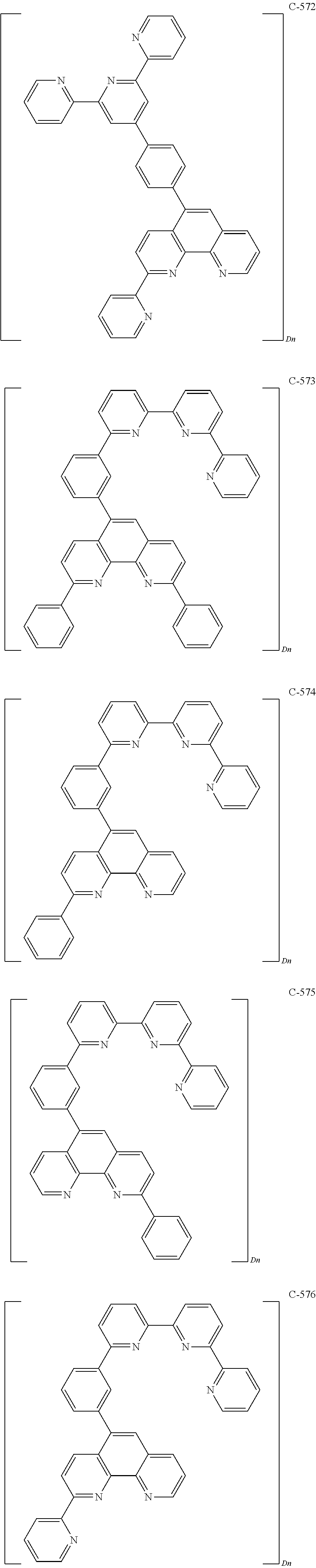

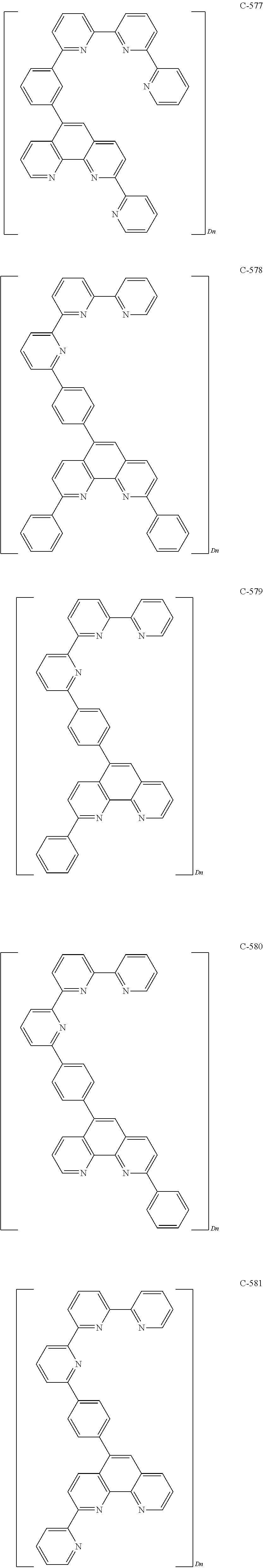

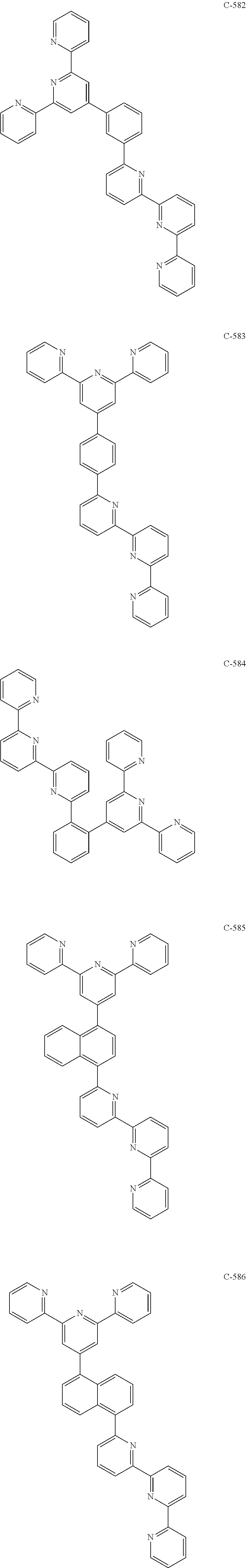 [SMILES: c1ccc(-c2cc(-c3cccc(-c4cccc(-c5cccc(-c6ccccn6)n5)n4)c3)cc(-c3ccccn3)n2)nc1 ; c1ccc(-c2cc(-c3ccc(-c4cccc(-c5cccc(-c6ccccn6)n5)n4)cc3)cc(-c3ccccn3)n2)nc1 ; c1ccc(-c2cc(-c3ccccc3-c3cccc(-c4cccc(-c5ccccn5)n4)n3)cc(-c3ccccn3)n2)nc1 ; c1ccc(-c2cc(-c3ccc(-c4cccc(-c5cccc(-c6ccccn6)n5)n4)c4ccccc34)cc(-c3ccccn3)n2)nc1 ; c1ccc(-c2cc(-c3cccc4c(-c5cccc(-c6cccc(-c7ccccn7)n6)n5)cccc34)cc(-c3ccccn3)n2)nc1]

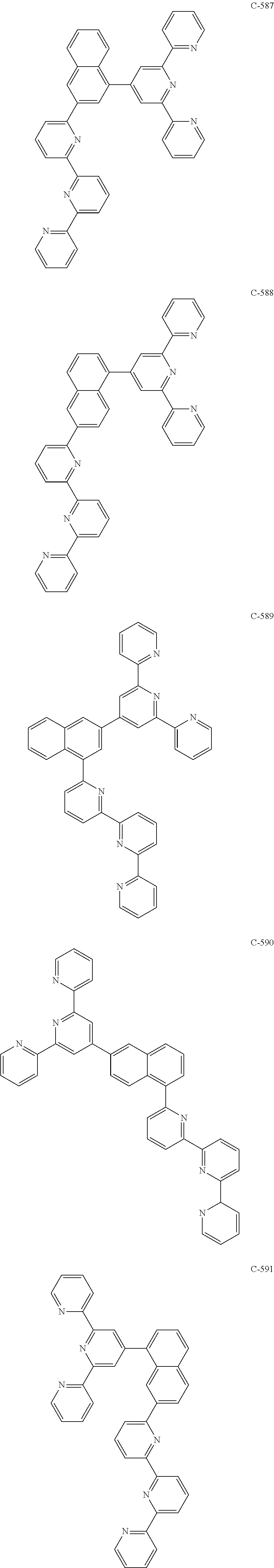

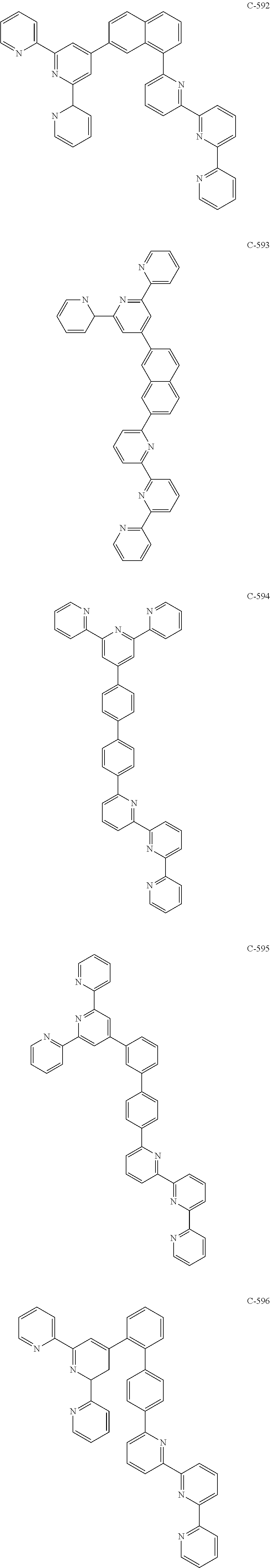

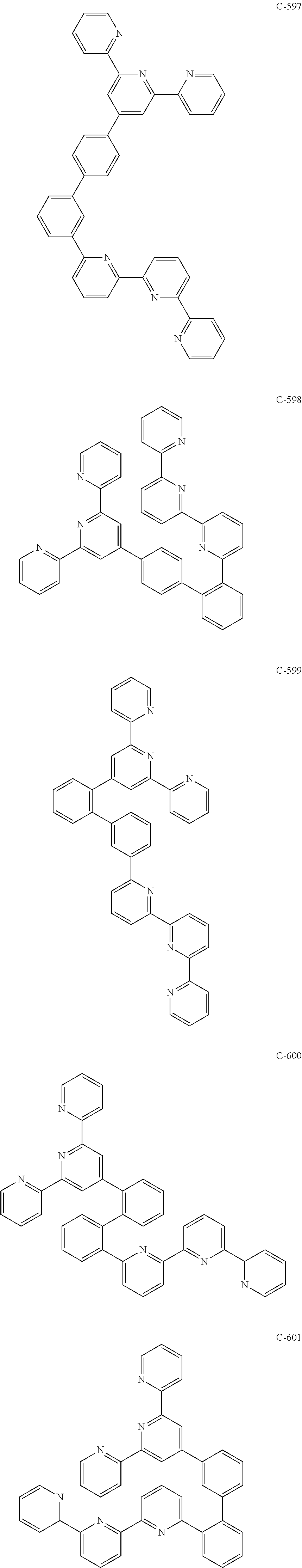

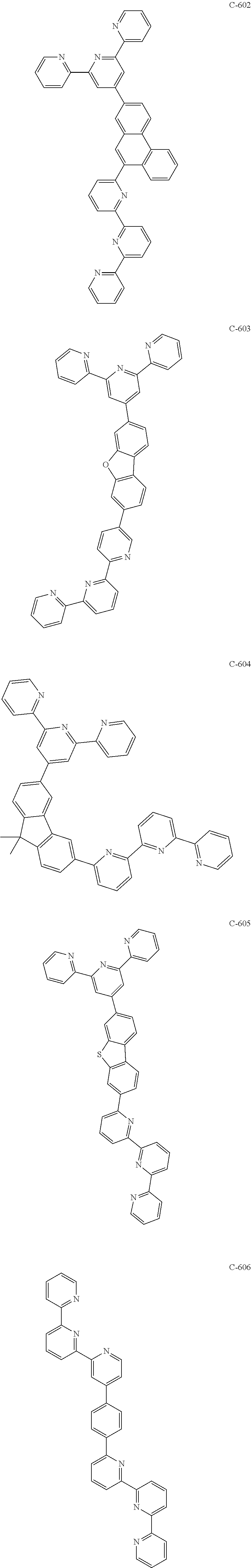 [SMILES: c1ccc(-c2cc(-c3ccc4c(c3)cc(-c3cccc(-c5cccc(-c6ccccn6)n5)n3)c3ccccc34)cc(-c3ccccn3)n2)nc1 ; c1ccc(-c2cccc(-c3ccc(-c4ccc5c(c4)oc4cc(-c6cc(-c7ccccn7)nc(-c7ccccn7)c6)ccc45)cn3)n2)nc1 ; CC1(C)c2ccc(-c3cc(-c4ccccn4)nc(-c4ccccn4)c3)cc2-c2cc(-c3cccc(-c4cccc(-c5ccccn5)n4)n3)ccc21 ; c1ccc(-c2cc(-c3ccc4c(c3)sc3cc(-c5cccc(-c6cccc(-c7ccccn7)n6)n5)ccc34)cc(-c3ccccn3)n2)nc1 ; c1ccc(-c2cccc(-c3cc(-c4ccc(-c5cccc(-c6cccc(-c7ccccn7)n6)n5)cc4)ccn3)n2)nc1]

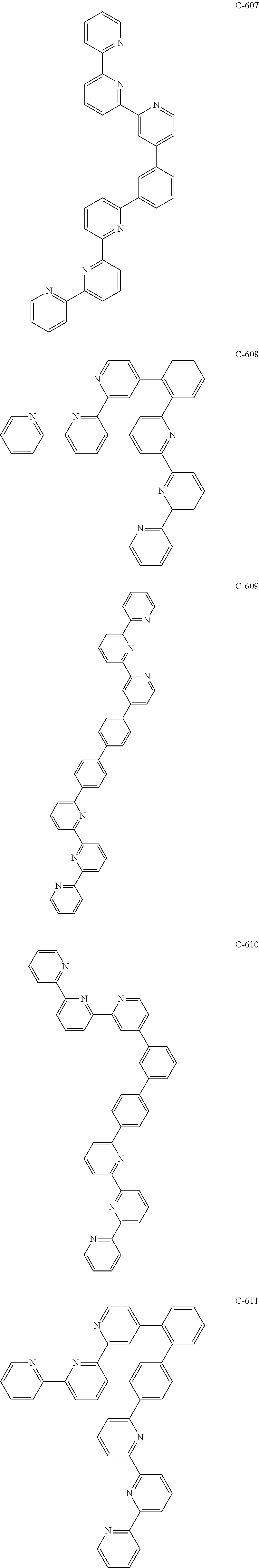

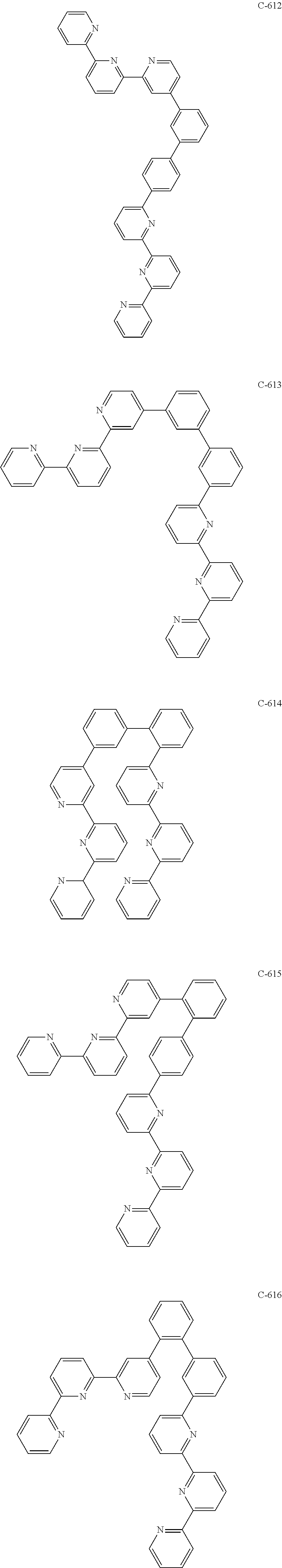

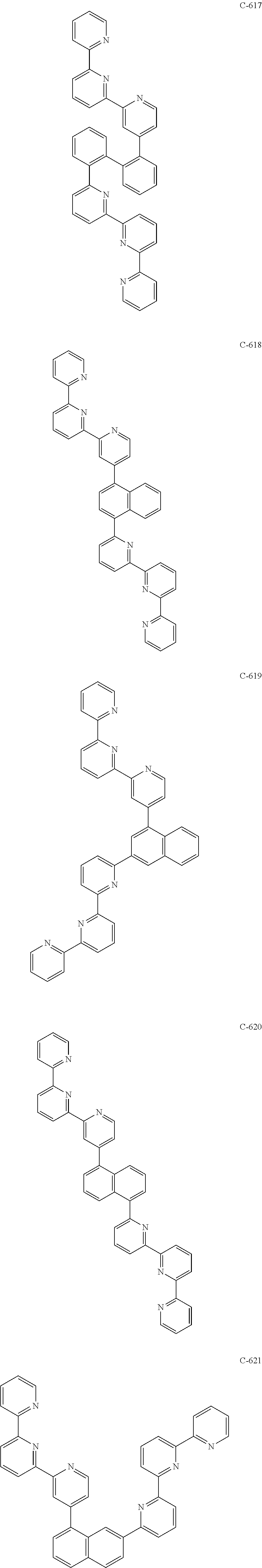 [SMILES: c1ccc(-c2cccc(-c3cc(-c4ccccc4-c4ccccc4-c4cccc(-c5cccc(-c6ccccn6)n5)n4)ccn3)n2)nc1 ; c1ccc(-c2cccc(-c3cc(-c4ccc(-c5cccc(-c6cccc(-c7ccccn7)n6)n5)c5ccccc45)ccn3)n2)nc1 ; c1ccc(-c2cccc(-c3cc(-c4cc(-c5cccc(-c6cccc(-c7ccccn7)n6)n5)cc5ccccc45)ccn3)n2)nc1 ; c1ccc(-c2cccc(-c3cc(-c4cccc5c(-c6cccc(-c7cccc(-c8ccccn8)n7)n6)cccc45)ccn3)n2)nc1 ; c1ccc(-c2cccc(-c3cc(-c4cccc5ccc(-c6cccc(-c7cccc(-c8ccccn8)n7)n6)cc45)ccn3)n2)nc1]

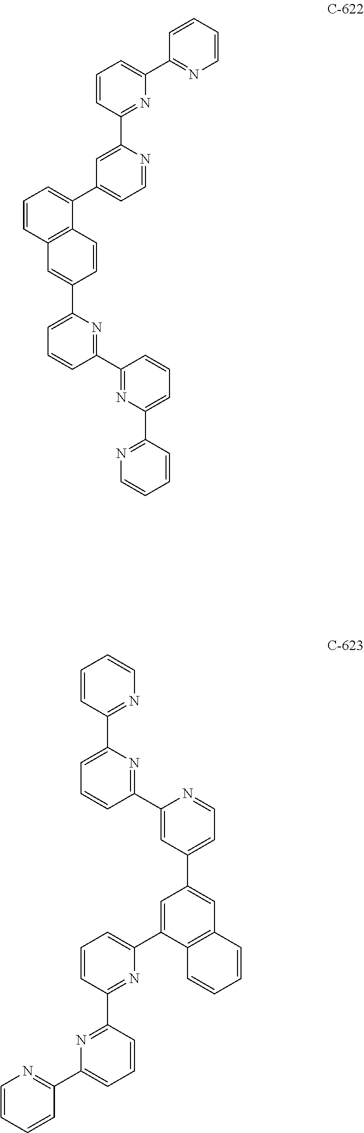 [SMILES: c1ccc(-c2cccc(-c3cc(-c4cccc5cc(-c6cccc(-c7cccc(-c8ccccn8)n7)n6)ccc45)ccn3)n2)nc1 ; c1ccc(-c2cccc(-c3cc(-c4cc(-c5cccc(-c6cccc(-c7ccccn7)n6)n5)c5ccccc5c4)ccn3)n2)nc1]

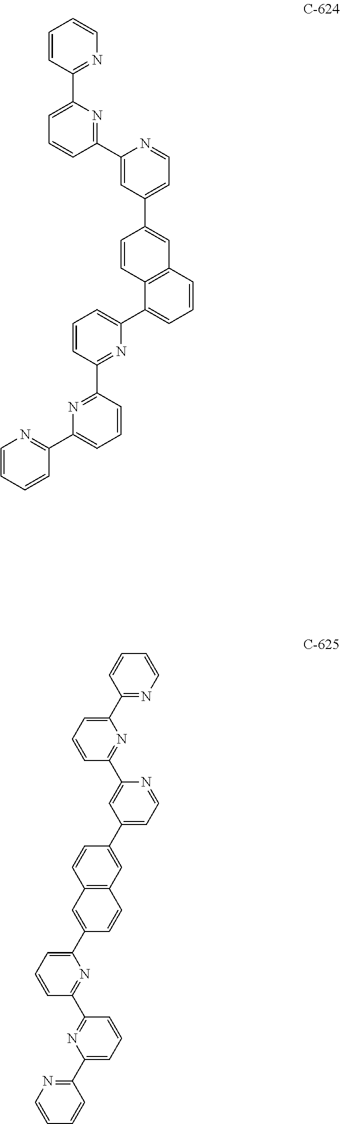 [SMILES: c1ccc(-c2cccc(-c3cc(-c4ccc5c(-c6cccc(-c7cccc(-c8ccccn8)n7)n6)cccc5c4)ccn3)n2)nc1 ; c1ccc(-c2cccc(-c3cc(-c4ccc5cc(-c6cccc(-c7cccc(-c8ccccn8)n7)n6)ccc5c4)ccn3)n2)nc1]

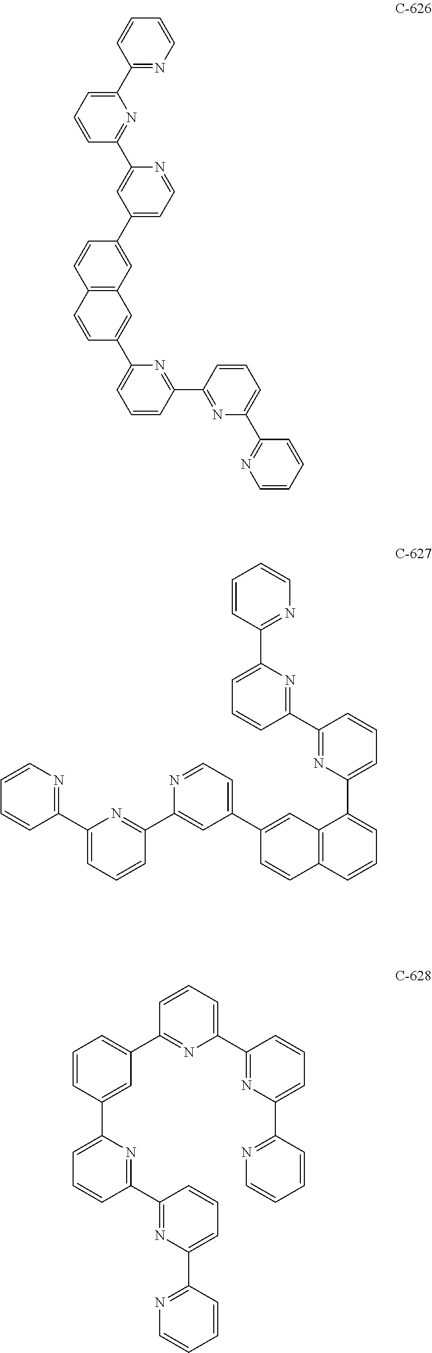 [SMILES: c1ccc(-c2cccc(-c3cc(-c4ccc5ccc(-c6cccc(-c7cccc(-c8ccccn8)n7)n6)cc5c4)ccn3)n2)nc1 ; c1ccc(-c2cccc(-c3cc(-c4ccc5cccc(-c6cccc(-c7cccc(-c8ccccn8)n7)n6)c5c4)ccn3)n2)nc1 ; c1ccc(-c2cccc(-c3cccc(-c4cccc(-c5cccc(-c6cccc(-c7ccccn7)n6)n5)c4)n3)n2)nc1]

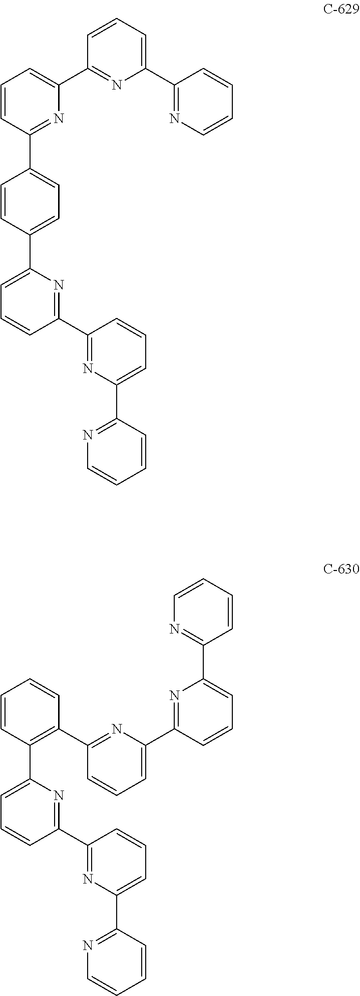 [SMILES: c1ccc(-c2cccc(-c3cccc(-c4ccc(-c5cccc(-c6cccc(-c7ccccn7)n6)n5)cc4)n3)n2)nc1 ; c1ccc(-c2cccc(-c3cccc(-c4ccccc4-c4cccc(-c5cccc(-c6ccccn6)n5)n4)n3)n2)nc1]

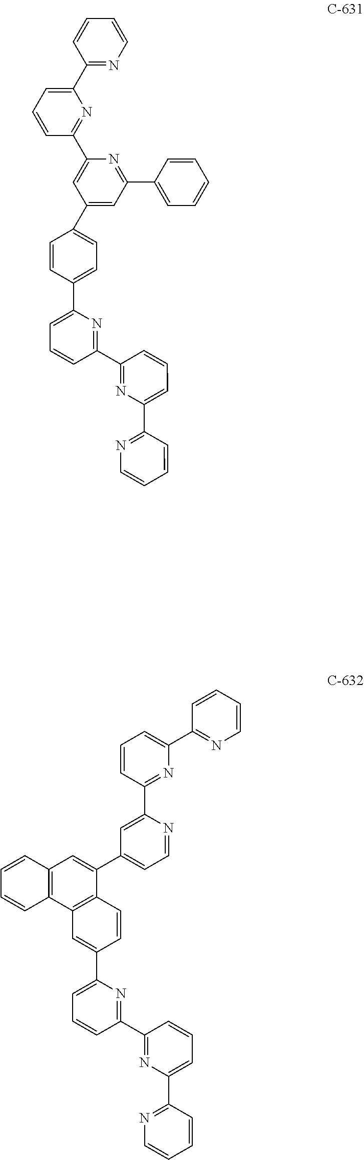 [SMILES: c1ccc(-c2cc(-c3ccc(-c4cccc(-c5cccc(-c6ccccn6)n5)n4)cc3)cc(-c3cccc(-c4ccccn4)n3)n2)cc1 ; c1ccc(-c2cccc(-c3cc(-c4cc5ccccc5c5cc(-c6cccc(-c7cccc(-c8ccccn8)n7)n6)ccc45)ccn3)n2)nc1]

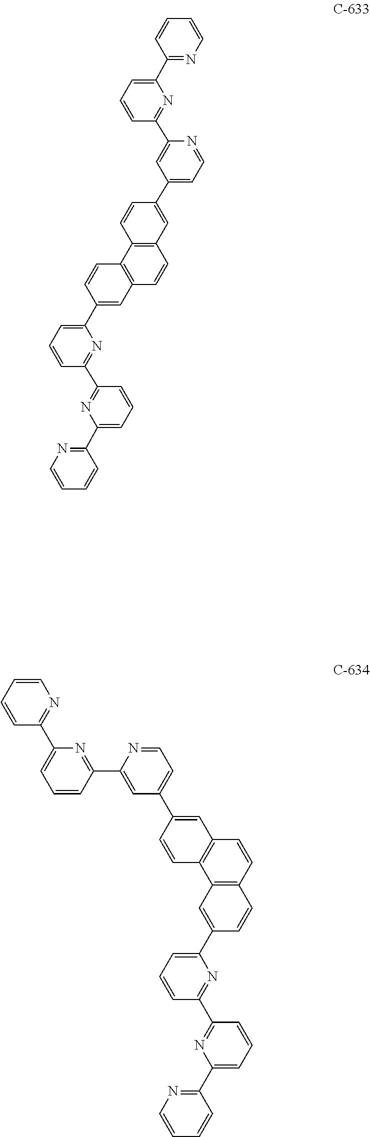 [SMILES: c1ccc(-c2cccc(-c3cc(-c4ccc5c(ccc6cc(-c7cccc(-c8cccc(-c9ccccn9)n8)n7)ccc65)c4)ccn3)n2)nc1 ; c1ccc(-c2cccc(-c3cc(-c4ccc5c(ccc6ccc(-c7cccc(-c8cccc(-c9ccccn9)n8)n7)cc65)c4)ccn3)n2)nc1]

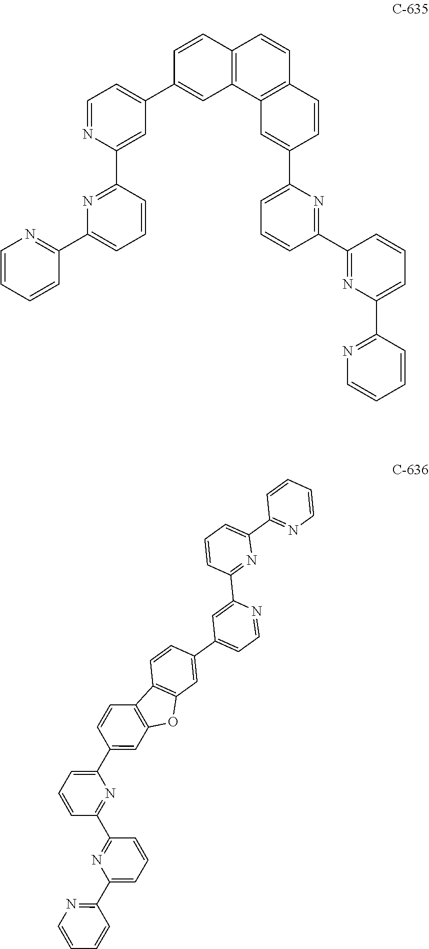 [SMILES: c1ccc(-c2cccc(-c3cc(-c4ccc5ccc6ccc(-c7cccc(-c8cccc(-c9ccccn9)n8)n7)cc6c5c4)ccn3)n2)nc1 ; c1ccc(-c2cccc(-c3cc(-c4ccc5c(c4)oc4cc(-c6cccc(-c7cccc(-c8ccccn8)n7)n6)ccc45)ccn3)n2)nc1]

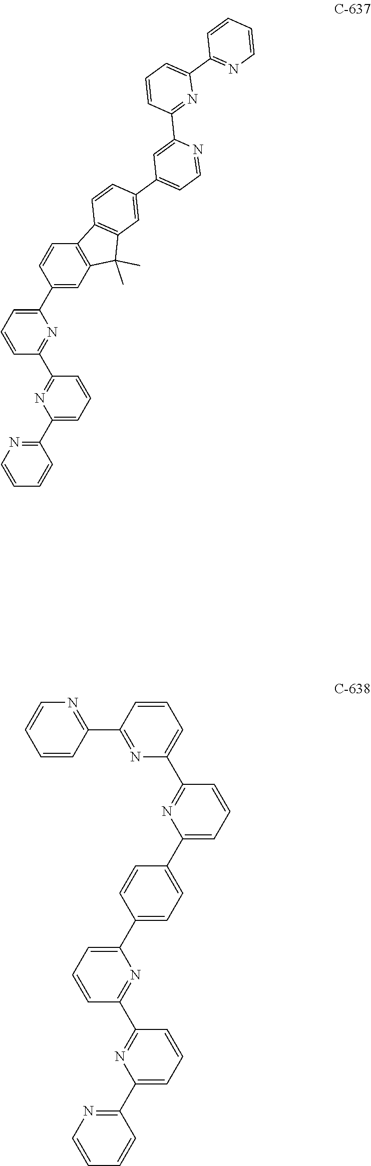 [SMILES: CC1(C)c2cc(-c3ccnc(-c4cccc(-c5ccccn5)n4)c3)ccc2-c2ccc(-c3cccc(-c4cccc(-c5ccccn5)n4)n3)cc21 ; c1ccc(-c2cccc(-c3cccc(-c4ccc(-c5cccc(-c6cccc(-c7ccccn7)n6)n5)cc4)n3)n2)nc1]

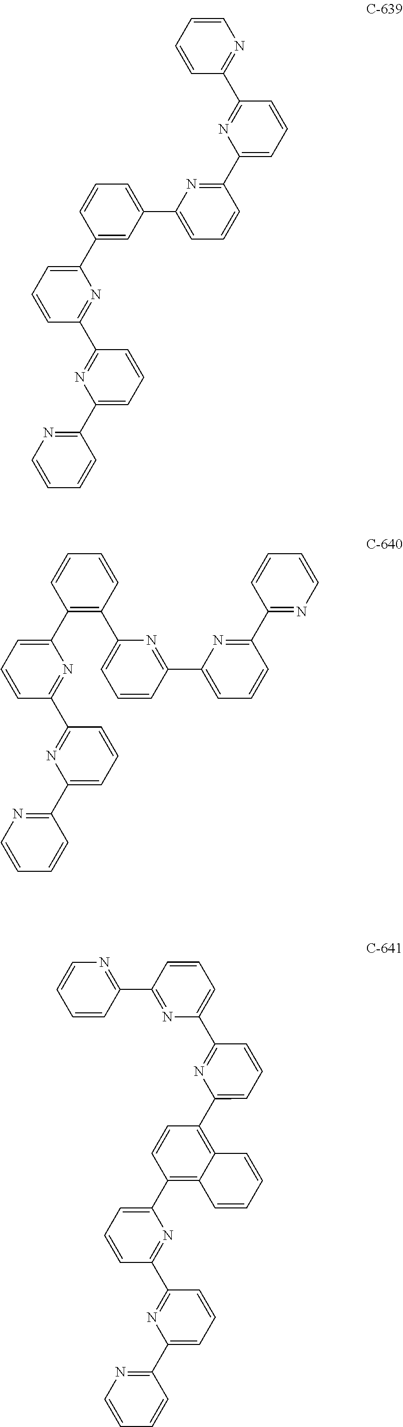 [SMILES: c1ccc(-c2cccc(-c3cccc(-c4cccc(-c5cccc(-c6cccc(-c7ccccn7)n6)n5)c4)n3)n2)nc1 ; c1ccc(-c2cccc(-c3cccc(-c4ccccc4-c4cccc(-c5cccc(-c6ccccn6)n5)n4)n3)n2)nc1 ; c1ccc(-c2cccc(-c3cccc(-c4ccc(-c5cccc(-c6cccc(-c7ccccn7)n6)n5)c5ccccc45)n3)n2)nc1]

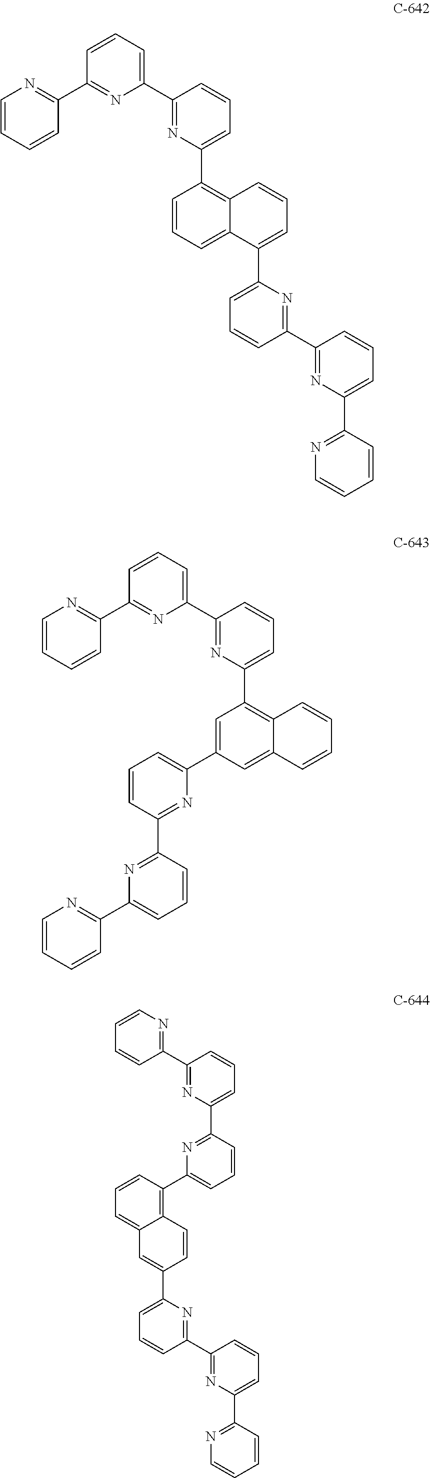 [SMILES: c1ccc(-c2cccc(-c3cccc(-c4cccc5c(-c6cccc(-c7cccc(-c8ccccn8)n7)n6)cccc45)n3)n2)nc1 ; c1ccc(-c2cccc(-c3cccc(-c4cc(-c5cccc(-c6cccc(-c7ccccn7)n6)n5)c5ccccc5c4)n3)n2)nc1 ; c1ccc(-c2cccc(-c3cccc(-c4ccc5c(-c6cccc(-c7cccc(-c8ccccn8)n7)n6)cccc5c4)n3)n2)nc1]

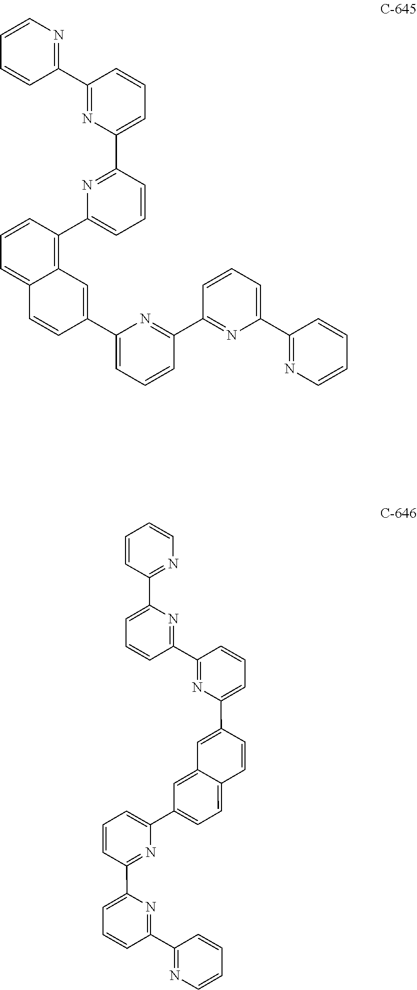 [SMILES: c1ccc(-c2cccc(-c3cccc(-c4ccc5cccc(-c6cccc(-c7cccc(-c8ccccn8)n7)n6)c5c4)n3)n2)nc1 ; c1ccc(-c2cccc(-c3cccc(-c4ccc5ccc(-c6cccc(-c7cccc(-c8ccccn8)n7)n6)cc5c4)n3)n2)nc1]

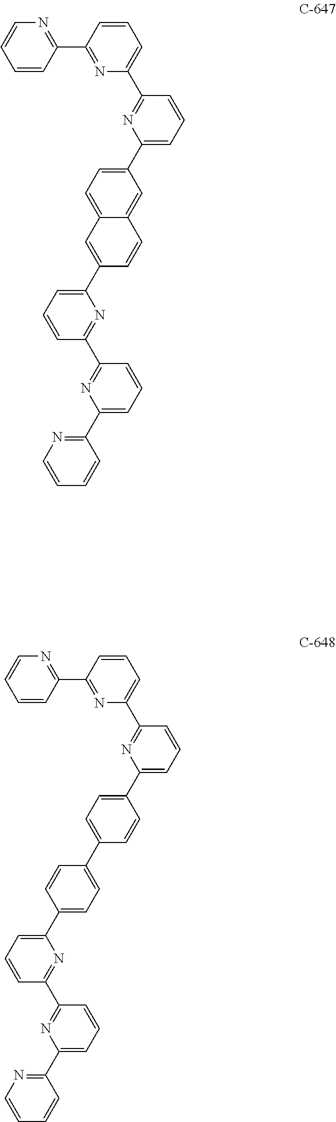 [SMILES: c1ccc(-c2cccc(-c3cccc(-c4ccc5cc(-c6cccc(-c7cccc(-c8ccccn8)n7)n6)ccc5c4)n3)n2)nc1 ; c1ccc(-c2cccc(-c3cccc(-c4ccc(-c5ccc(-c6cccc(-c7cccc(-c8ccccn8)n7)n6)cc5)cc4)n3)n2)nc1]

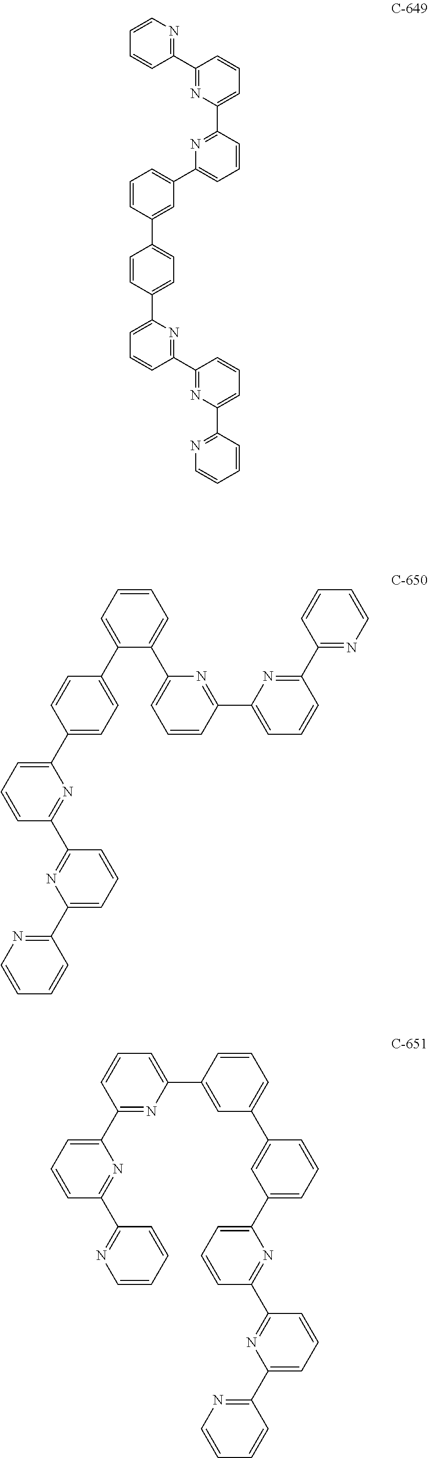 [SMILES: c1ccc(-c2cccc(-c3cccc(-c4ccc(-c5cccc(-c6cccc(-c7cccc(-c8ccccn8)n7)n6)c5)cc4)n3)n2)nc1 ; c1ccc(-c2cccc(-c3cccc(-c4ccc(-c5ccccc5-c5cccc(-c6cccc(-c7ccccn7)n6)n5)cc4)n3)n2)nc1 ; c1ccc(-c2cccc(-c3cccc(-c4cccc(-c5cccc(-c6cccc(-c7cccc(-c8ccccn8)n7)n6)c5)c4)n3)n2)nc1]

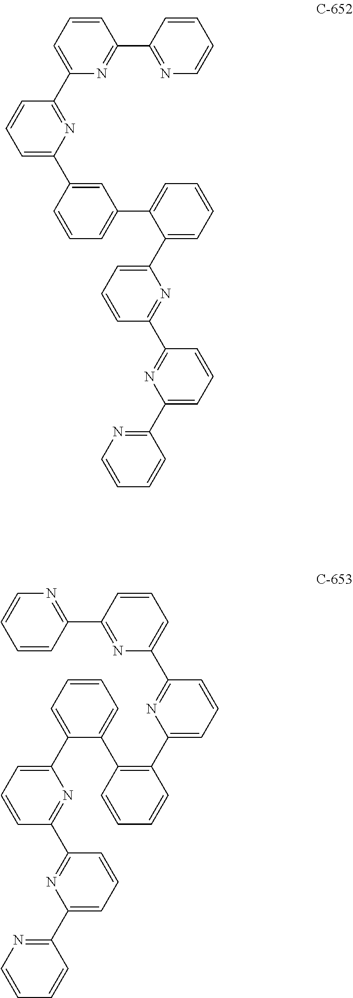 [SMILES: c1ccc(-c2cccc(-c3cccc(-c4cccc(-c5ccccc5-c5cccc(-c6cccc(-c7ccccn7)n6)n5)c4)n3)n2)nc1 ; c1ccc(-c2cccc(-c3cccc(-c4ccccc4-c4ccccc4-c4cccc(-c5cccc(-c6ccccn6)n5)n4)n3)n2)nc1]

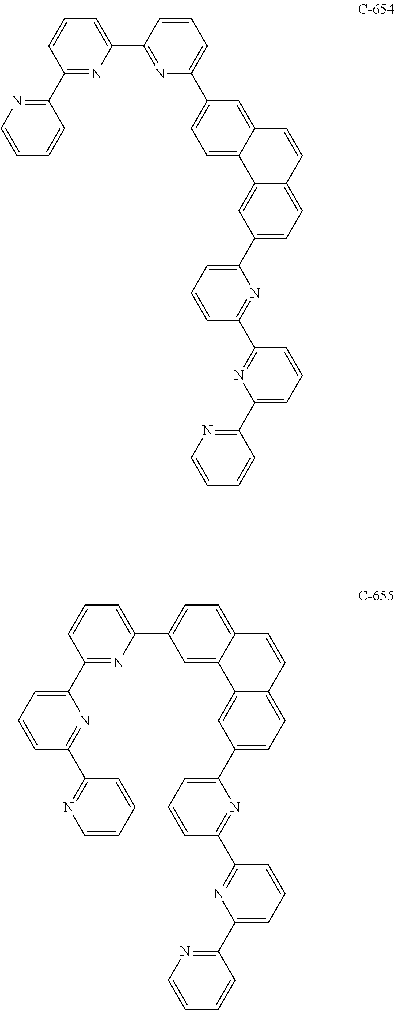 [SMILES: c1ccc(-c2cccc(-c3cccc(-c4ccc5c(ccc6ccc(-c7cccc(-c8cccc(-c9ccccn9)n8)n7)cc65)c4)n3)n2)nc1 ; c1ccc(-c2cccc(-c3cccc(-c4ccc5ccc6ccc(-c7cccc(-c8cccc(-c9ccccn9)n8)n7)cc6c5c4)n3)n2)nc1]

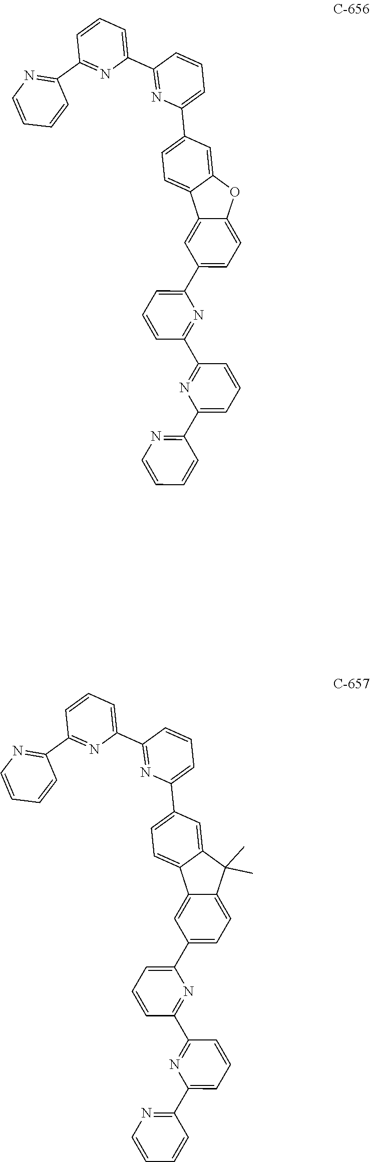 [SMILES: c1ccc(-c2cccc(-c3cccc(-c4ccc5c(c4)oc4ccc(-c6cccc(-c7cccc(-c8ccccn8)n7)n6)cc45)n3)n2)nc1 ; CC1(C)c2ccc(-c3cccc(-c4cccc(-c5ccccn5)n4)n3)cc2-c2ccc(-c3cccc(-c4cccc(-c5ccccn5)n4)n3)cc21]

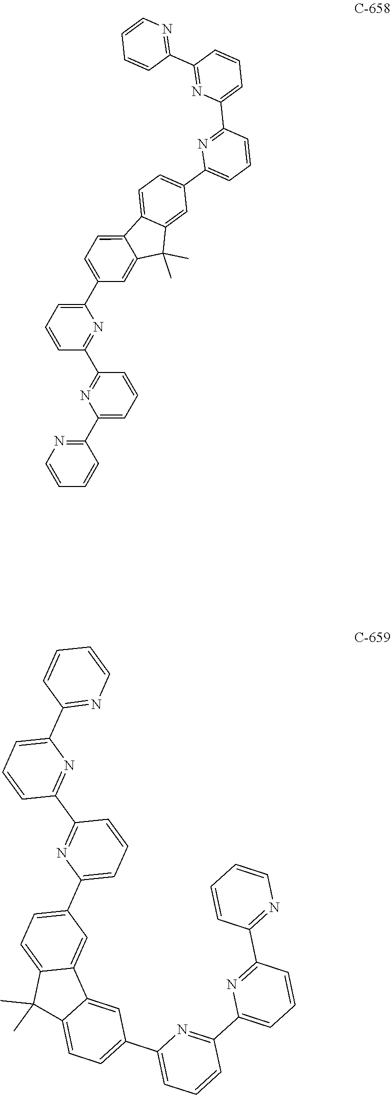 [SMILES: CC1(C)c2cc(-c3cccc(-c4cccc(-c5ccccn5)n4)n3)ccc2-c2ccc(-c3cccc(-c4cccc(-c5ccccn5)n4)n3)cc21 ; CC1(C)c2ccc(-c3cccc(-c4cccc(-c5ccccn5)n4)n3)cc2-c2cc(-c3cccc(-c4cccc(-c5ccccn5)n4)n3)ccc21]

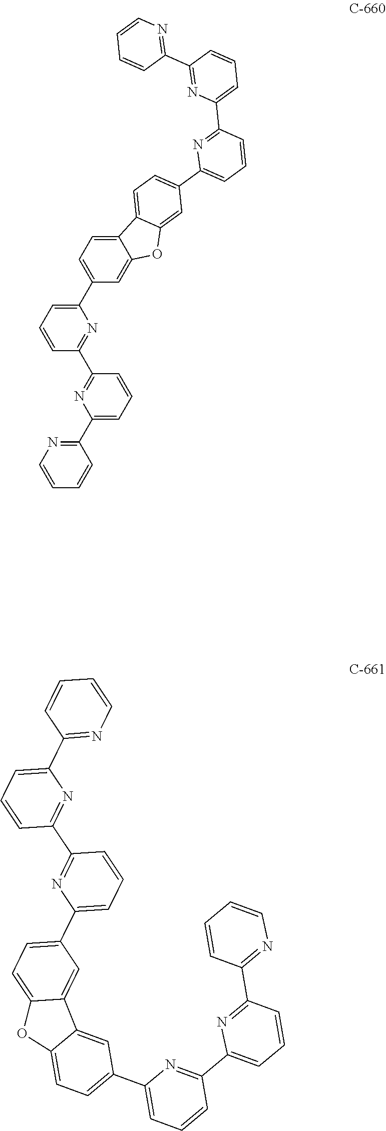 [SMILES: c1ccc(-c2cccc(-c3cccc(-c4ccc5c(c4)oc4cc(-c6cccc(-c7cccc(-c8ccccn8)n7)n6)ccc45)n3)n2)nc1 ; c1ccc(-c2cccc(-c3cccc(-c4ccc5oc6ccc(-c7cccc(-c8cccc(-c9ccccn9)n8)n7)cc6c5c4)n3)n2)nc1]

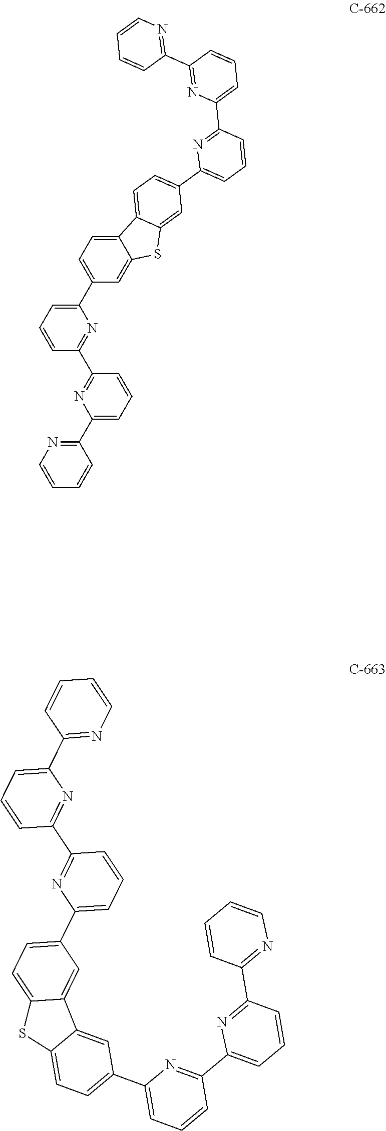 [SMILES: c1ccc(-c2cccc(-c3cccc(-c4ccc5c(c4)sc4cc(-c6cccc(-c7cccc(-c8ccccn8)n7)n6)ccc45)n3)n2)nc1 ; c1ccc(-c2cccc(-c3cccc(-c4ccc5sc6ccc(-c7cccc(-c8cccc(-c9ccccn9)n8)n7)cc6c5c4)n3)n2)nc1]

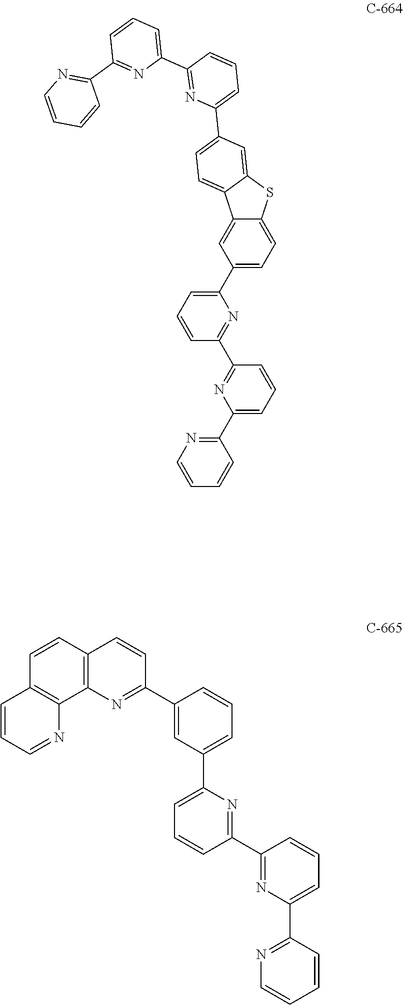 [SMILES: c1ccc(-c2cccc(-c3cccc(-c4ccc5c(c4)sc4ccc(-c6cccc(-c7cccc(-c8ccccn8)n7)n6)cc45)n3)n2)nc1 ; c1ccc(-c2cccc(-c3cccc(-c4cccc(-c5ccc6ccc7cccnc7c6n5)c4)n3)n2)nc1]

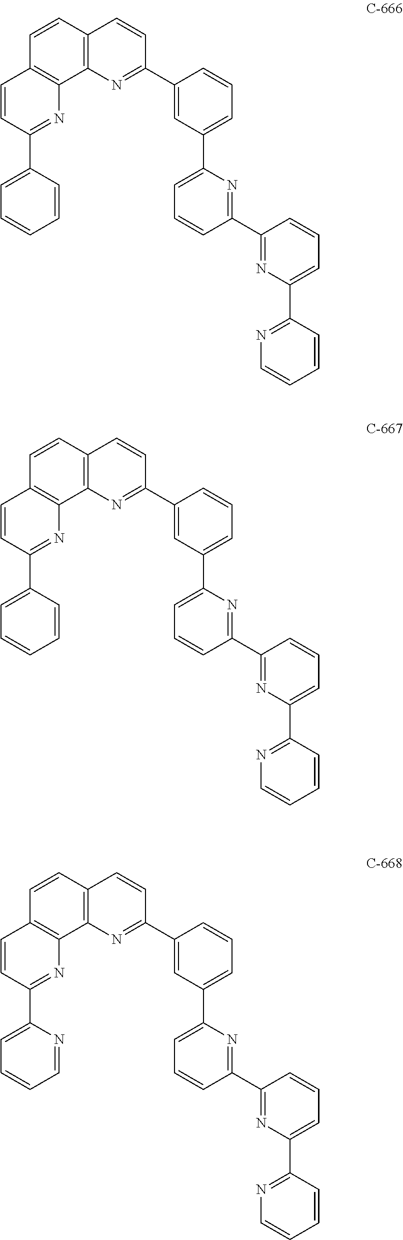 [SMILES: c1ccc(-c2ccc3ccc4ccc(-c5cccc(-c6cccc(-c7cccc(-c8ccccn8)n7)n6)c5)nc4c3n2)cc1 ; c1ccc(-c2ccc3ccc4ccc(-c5cccc(-c6cccc(-c7cccc(-c8ccccn8)n7)n6)c5)nc4c3n2)cc1 ; c1ccc(-c2cccc(-c3cccc(-c4cccc(-c5ccc6ccc7ccc(-c8ccccn8)nc7c6n5)c4)n3)n2)nc1]

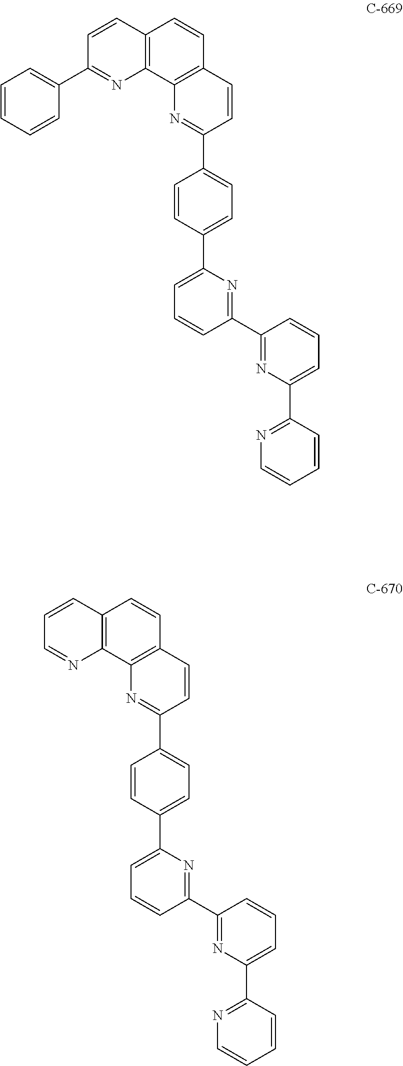 [SMILES: c1ccc(-c2ccc3ccc4ccc(-c5ccc(-c6cccc(-c7cccc(-c8ccccn8)n7)n6)cc5)nc4c3n2)cc1 ; c1ccc(-c2cccc(-c3cccc(-c4ccc(-c5ccc6ccc7cccnc7c6n5)cc4)n3)n2)nc1]

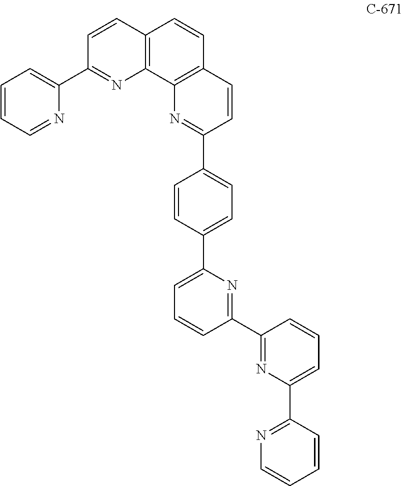 [SMILES: c1ccc(-c2cccc(-c3cccc(-c4ccc(-c5ccc6ccc7ccc(-c8ccccn8)nc7c6n5)cc4)n3)n2)nc1]

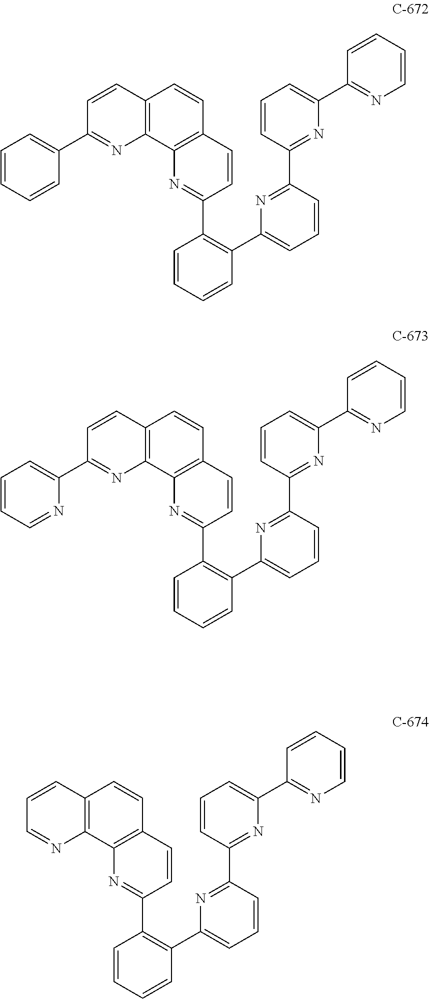 [SMILES: c1ccc(-c2ccc3ccc4ccc(-c5ccccc5-c5cccc(-c6cccc(-c7ccccn7)n6)n5)nc4c3n2)cc1 ; c1ccc(-c2cccc(-c3cccc(-c4ccccc4-c4ccc5ccc6ccc(-c7ccccn7)nc6c5n4)n3)n2)nc1 ; c1ccc(-c2cccc(-c3cccc(-c4ccccc4-c4ccc5ccc6cccnc6c5n4)n3)n2)nc1]

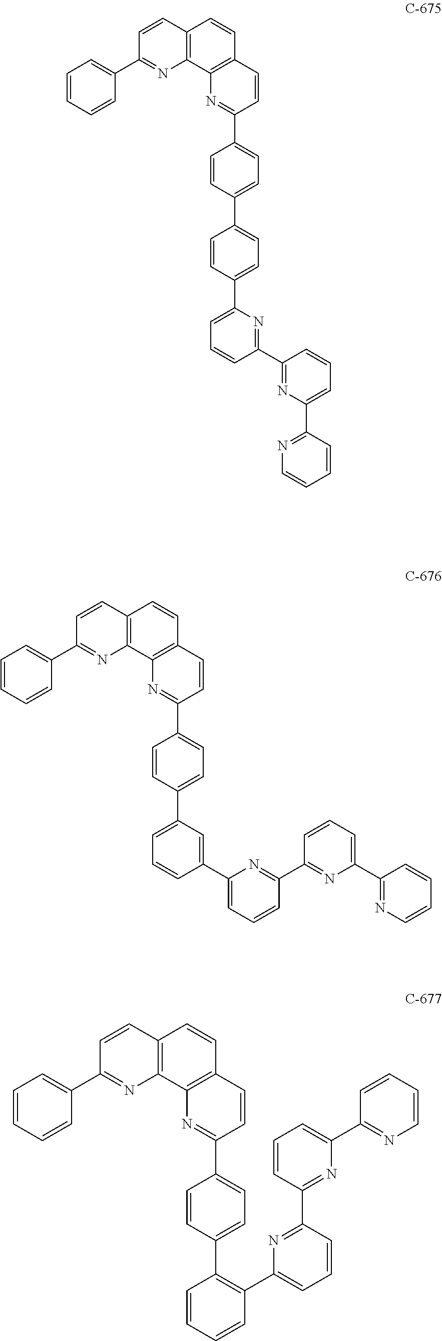 [SMILES: c1ccc(-c2ccc3ccc4ccc(-c5ccc(-c6ccc(-c7cccc(-c8cccc(-c9ccccn9)n8)n7)cc6)cc5)nc4c3n2)cc1 ; c1ccc(-c2ccc3ccc4ccc(-c5ccc(-c6cccc(-c7cccc(-c8cccc(-c9ccccn9)n8)n7)c6)cc5)nc4c3n2)cc1 ; c1ccc(-c2ccc3ccc4ccc(-c5ccc(-c6ccccc6-c6cccc(-c7cccc(-c8ccccn8)n7)n6)cc5)nc4c3n2)cc1]

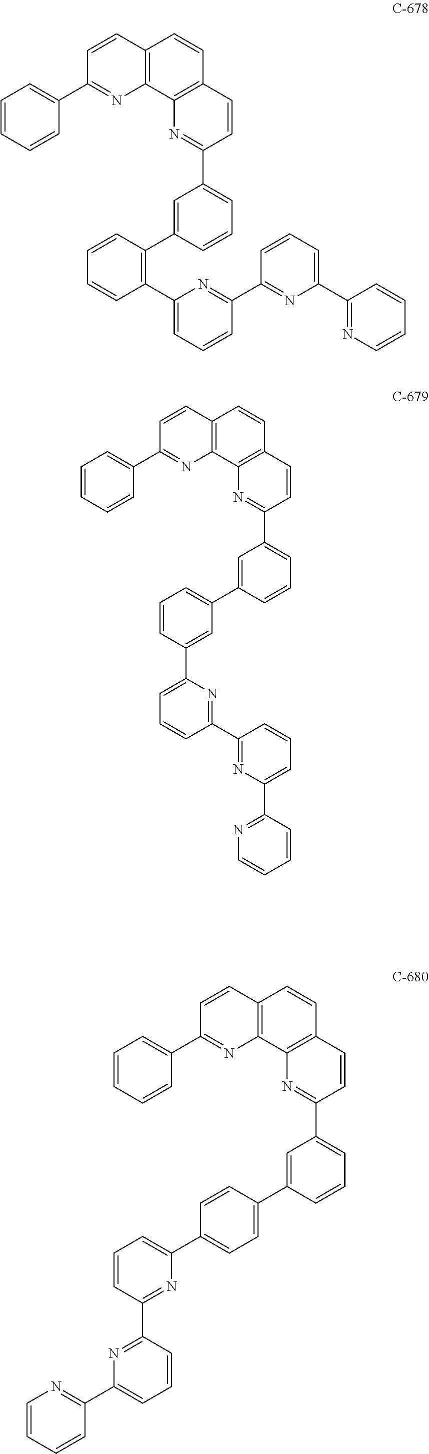 [SMILES: c1ccc(-c2ccc3ccc4ccc(-c5cccc(-c6ccccc6-c6cccc(-c7cccc(-c8ccccn8)n7)n6)c5)nc4c3n2)cc1 ; c1ccc(-c2ccc3ccc4ccc(-c5cccc(-c6cccc(-c7cccc(-c8cccc(-c9ccccn9)n8)n7)c6)c5)nc4c3n2)cc1 ; c1ccc(-c2ccc3ccc4ccc(-c5cccc(-c6ccc(-c7cccc(-c8cccc(-c9ccccn9)n8)n7)cc6)c5)nc4c3n2)cc1]

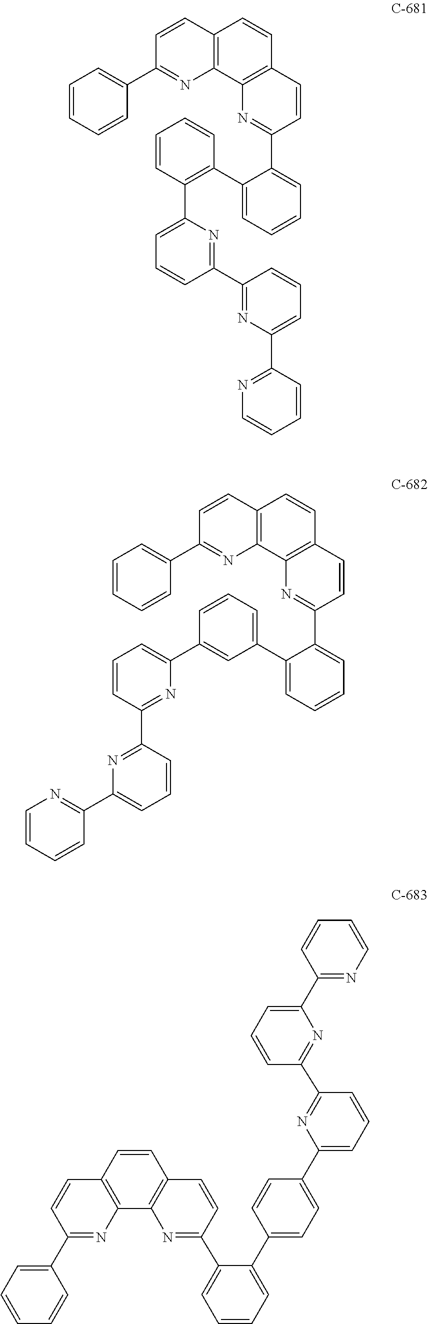 [SMILES: c1ccc(-c2ccc3ccc4ccc(-c5ccccc5-c5ccccc5-c5cccc(-c6cccc(-c7ccccn7)n6)n5)nc4c3n2)cc1 ; c1ccc(-c2ccc3ccc4ccc(-c5ccccc5-c5cccc(-c6cccc(-c7cccc(-c8ccccn8)n7)n6)c5)nc4c3n2)cc1 ; c1ccc(-c2ccc3ccc4ccc(-c5ccccc5-c5ccc(-c6cccc(-c7cccc(-c8ccccn8)n7)n6)cc5)nc4c3n2)cc1]

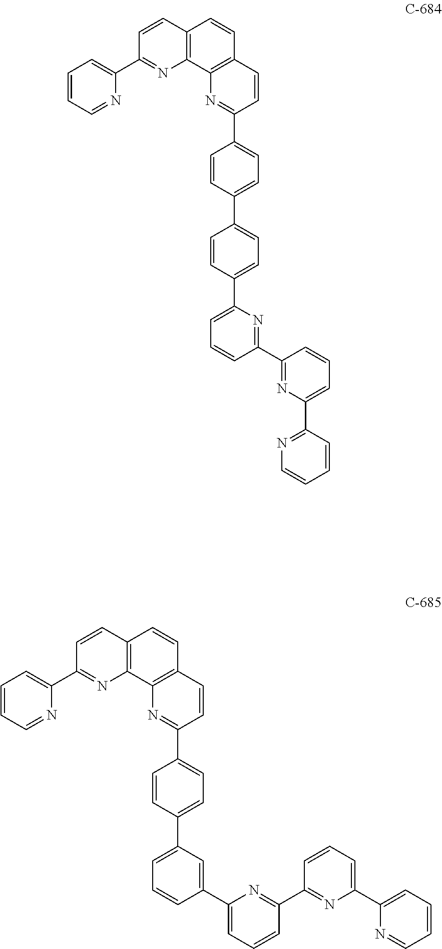 [SMILES: c1ccc(-c2cccc(-c3cccc(-c4ccc(-c5ccc(-c6ccc7ccc8ccc(-c9ccccn9)nc8c7n6)cc5)cc4)n3)n2)nc1 ; c1ccc(-c2cccc(-c3cccc(-c4cccc(-c5ccc(-c6ccc7ccc8ccc(-c9ccccn9)nc8c7n6)cc5)c4)n3)n2)nc1]

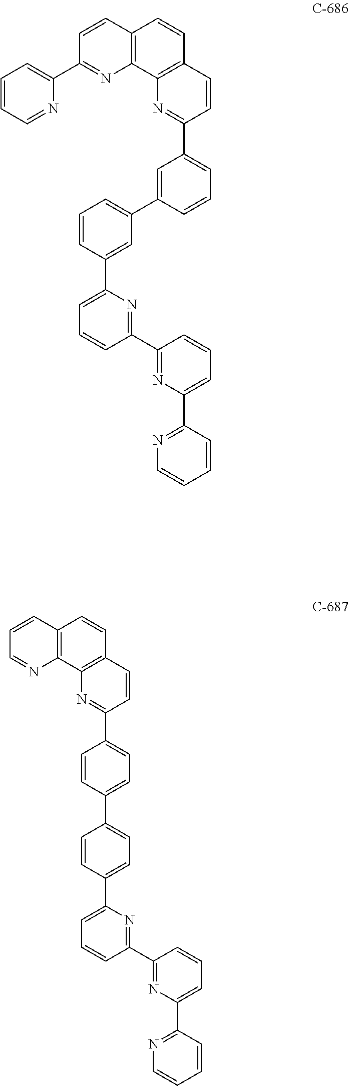 [SMILES: c1ccc(-c2cccc(-c3cccc(-c4cccc(-c5cccc(-c6ccc7ccc8ccc(-c9ccccn9)nc8c7n6)c5)c4)n3)n2)nc1 ; c1ccc(-c2cccc(-c3cccc(-c4ccc(-c5ccc(-c6ccc7ccc8cccnc8c7n6)cc5)cc4)n3)n2)nc1]

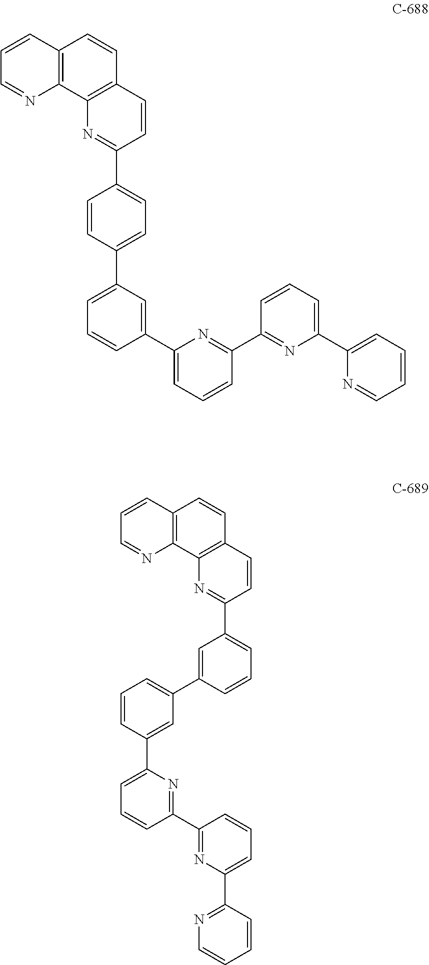 [SMILES: c1ccc(-c2cccc(-c3cccc(-c4cccc(-c5ccc(-c6ccc7ccc8cccnc8c7n6)cc5)c4)n3)n2)nc1 ; c1ccc(-c2cccc(-c3cccc(-c4cccc(-c5cccc(-c6ccc7ccc8cccnc8c7n6)c5)c4)n3)n2)nc1]

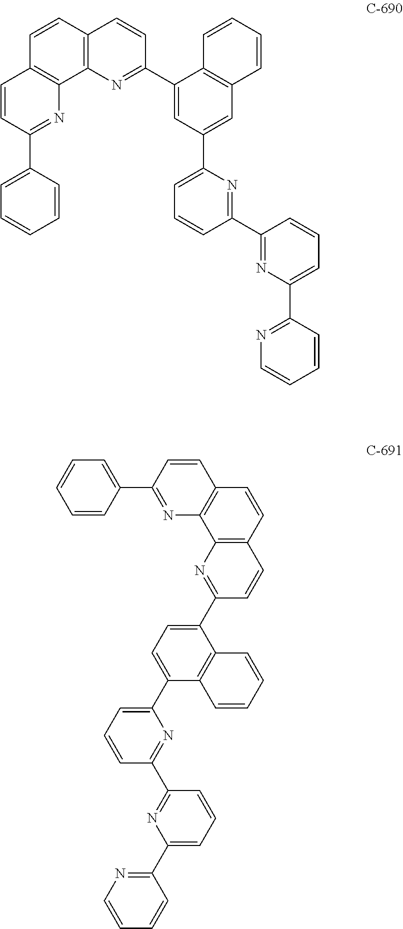 [SMILES: c1ccc(-c2ccc3ccc4ccc(-c5cc(-c6cccc(-c7cccc(-c8ccccn8)n7)n6)cc6ccccc56)nc4c3n2)cc1 ; c1ccc(-c2ccc3ccc4ccc(-c5ccc(-c6cccc(-c7cccc(-c8ccccn8)n7)n6)c6ccccc56)nc4c3n2)cc1]

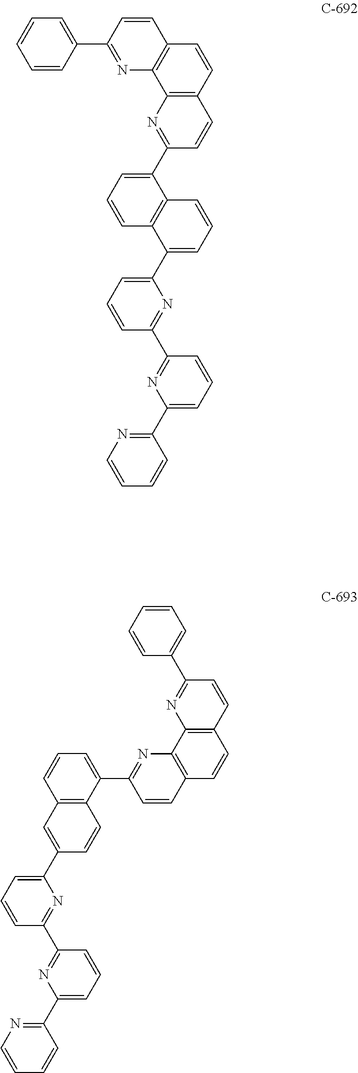 [SMILES: c1ccc(-c2ccc3ccc4ccc(-c5cccc6c(-c7cccc(-c8cccc(-c9ccccn9)n8)n7)cccc56)nc4c3n2)cc1 ; c1ccc(-c2ccc3ccc4ccc(-c5cccc6cc(-c7cccc(-c8cccc(-c9ccccn9)n8)n7)ccc56)nc4c3n2)cc1]

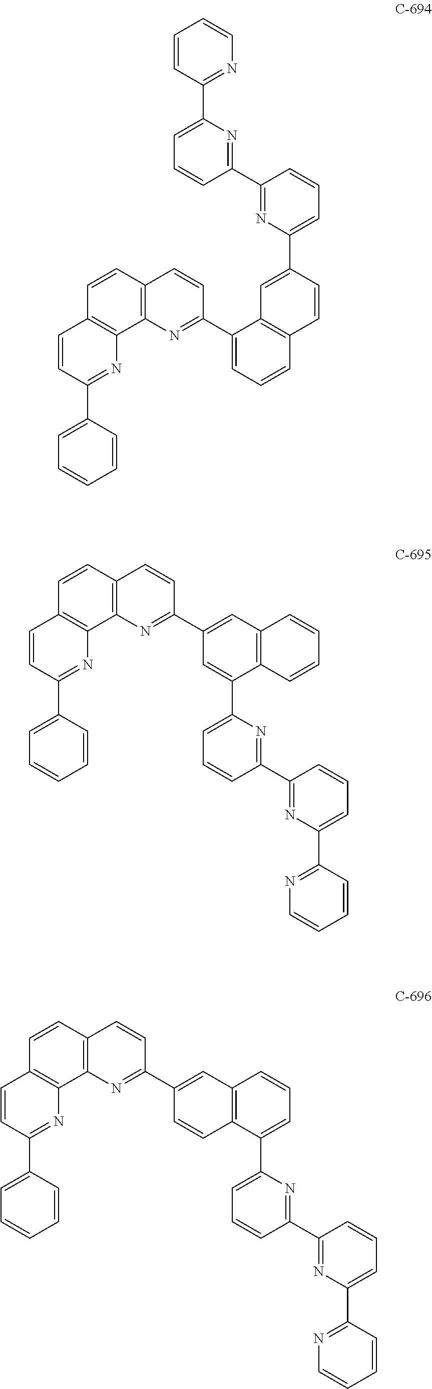 [SMILES: c1ccc(-c2ccc3ccc4ccc(-c5cccc6ccc(-c7cccc(-c8cccc(-c9ccccn9)n8)n7)cc56)nc4c3n2)cc1 ; c1ccc(-c2ccc3ccc4ccc(-c5cc(-c6cccc(-c7cccc(-c8ccccn8)n7)n6)c6ccccc6c5)nc4c3n2)cc1 ; c1ccc(-c2ccc3ccc4ccc(-c5ccc6c(-c7cccc(-c8cccc(-c9ccccn9)n8)n7)cccc6c5)nc4c3n2)cc1]

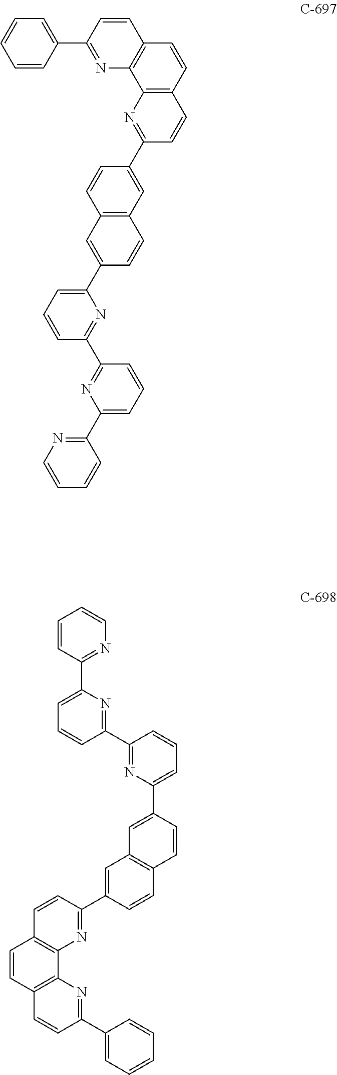 [SMILES: c1ccc(-c2ccc3ccc4ccc(-c5ccc6cc(-c7cccc(-c8cccc(-c9ccccn9)n8)n7)ccc6c5)nc4c3n2)cc1 ; c1ccc(-c2ccc3ccc4ccc(-c5ccc6ccc(-c7cccc(-c8cccc(-c9ccccn9)n8)n7)cc6c5)nc4c3n2)cc1]

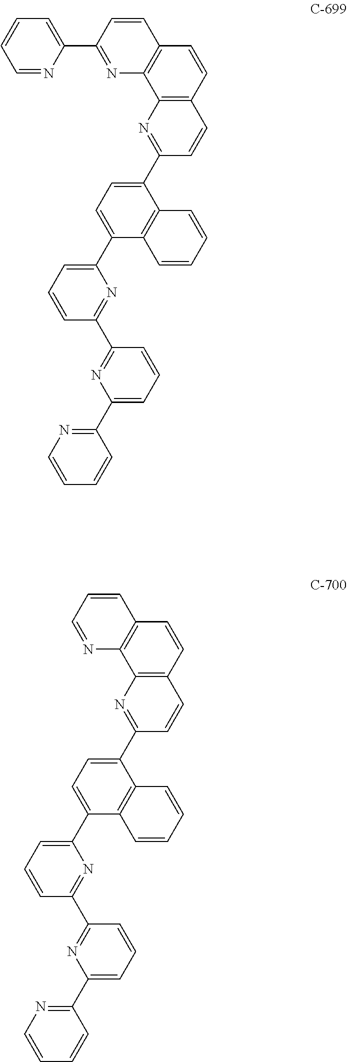 [SMILES: c1ccc(-c2cccc(-c3cccc(-c4ccc(-c5ccc6ccc7ccc(-c8ccccn8)nc7c6n5)c5ccccc45)n3)n2)nc1 ; c1ccc(-c2cccc(-c3cccc(-c4ccc(-c5ccc6ccc7cccnc7c6n5)c5ccccc45)n3)n2)nc1]

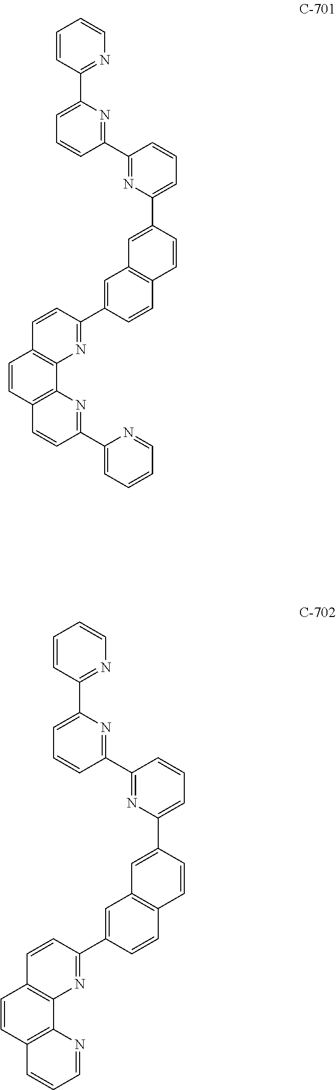 [SMILES: c1ccc(-c2cccc(-c3cccc(-c4ccc5ccc(-c6ccc7ccc8ccc(-c9ccccn9)nc8c7n6)cc5c4)n3)n2)nc1 ; c1ccc(-c2cccc(-c3cccc(-c4ccc5ccc(-c6ccc7ccc8cccnc8c7n6)cc5c4)n3)n2)nc1]

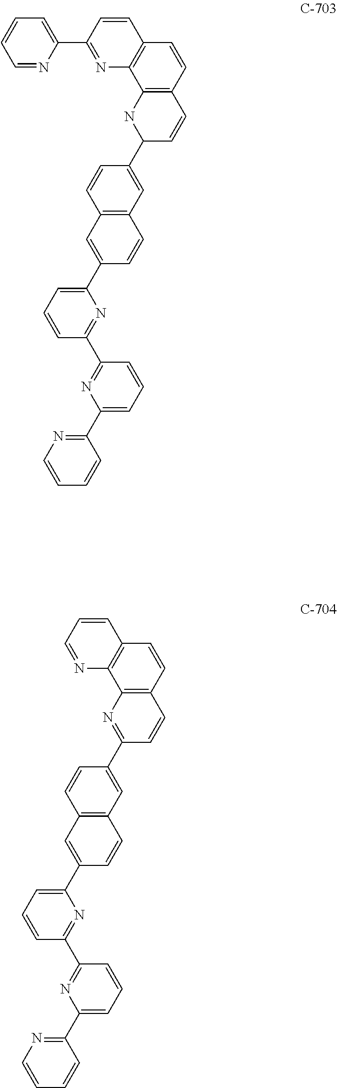

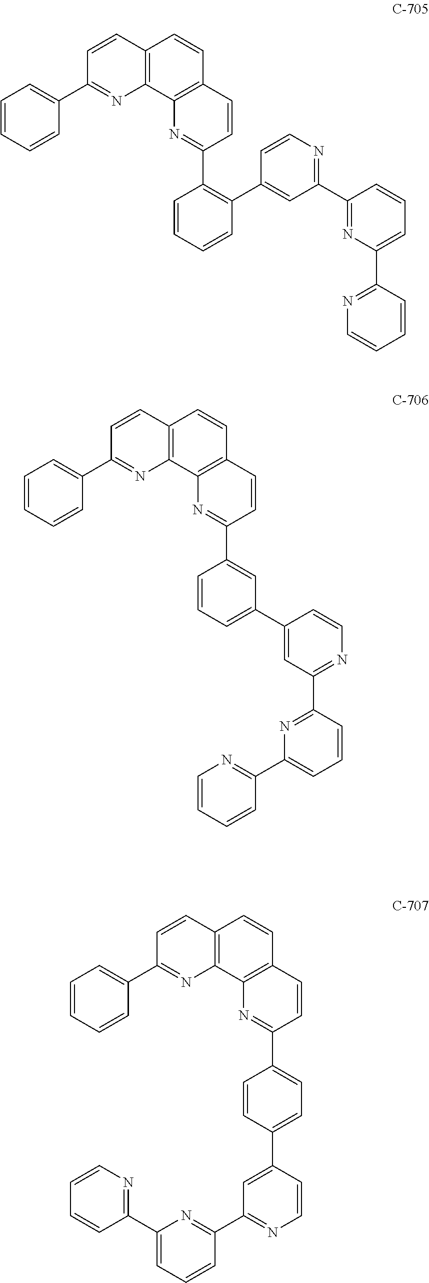 [SMILES: c1ccc(-c2ccc3ccc4ccc(-c5ccccc5-c5ccnc(-c6cccc(-c7ccccn7)n6)c5)nc4c3n2)cc1 ; c1ccc(-c2ccc3ccc4ccc(-c5cccc(-c6ccnc(-c7cccc(-c8ccccn8)n7)c6)c5)nc4c3n2)cc1 ; c1ccc(-c2ccc3ccc4ccc(-c5ccc(-c6ccnc(-c7cccc(-c8ccccn8)n7)c6)cc5)nc4c3n2)cc1]

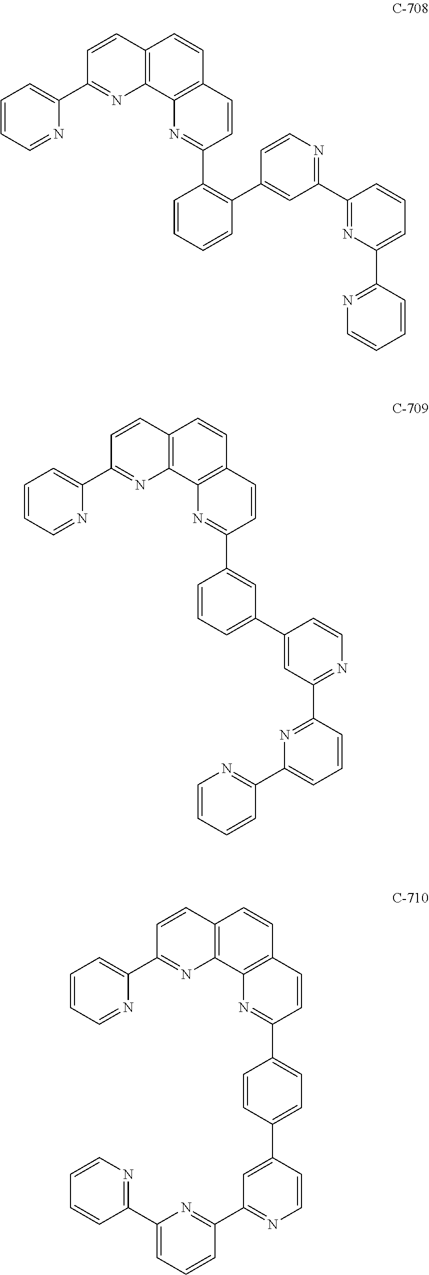 [SMILES: c1ccc(-c2cccc(-c3cc(-c4ccccc4-c4ccc5ccc6ccc(-c7ccccn7)nc6c5n4)ccn3)n2)nc1 ; c1ccc(-c2cccc(-c3cc(-c4cccc(-c5ccc6ccc7ccc(-c8ccccn8)nc7c6n5)c4)ccn3)n2)nc1 ; c1ccc(-c2cccc(-c3cc(-c4ccc(-c5ccc6ccc7ccc(-c8ccccn8)nc7c6n5)cc4)ccn3)n2)nc1]

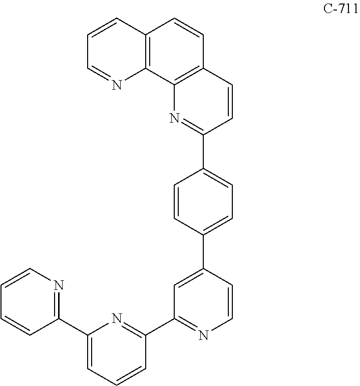 [SMILES: c1ccc(-c2cccc(-c3cc(-c4ccc(-c5ccc6ccc7cccnc7c6n5)cc4)ccn3)n2)nc1]

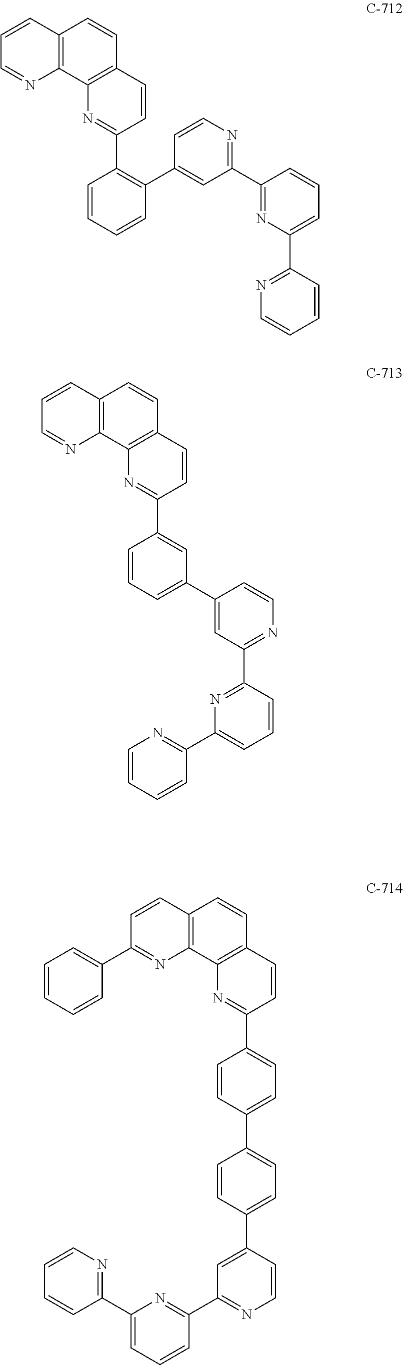 [SMILES: c1ccc(-c2cccc(-c3cc(-c4ccccc4-c4ccc5ccc6cccnc6c5n4)ccn3)n2)nc1 ; c1ccc(-c2cccc(-c3cc(-c4cccc(-c5ccc6ccc7cccnc7c6n5)c4)ccn3)n2)nc1 ; c1ccc(-c2ccc3ccc4ccc(-c5ccc(-c6ccc(-c7ccnc(-c8cccc(-c9ccccn9)n8)c7)cc6)cc5)nc4c3n2)cc1]

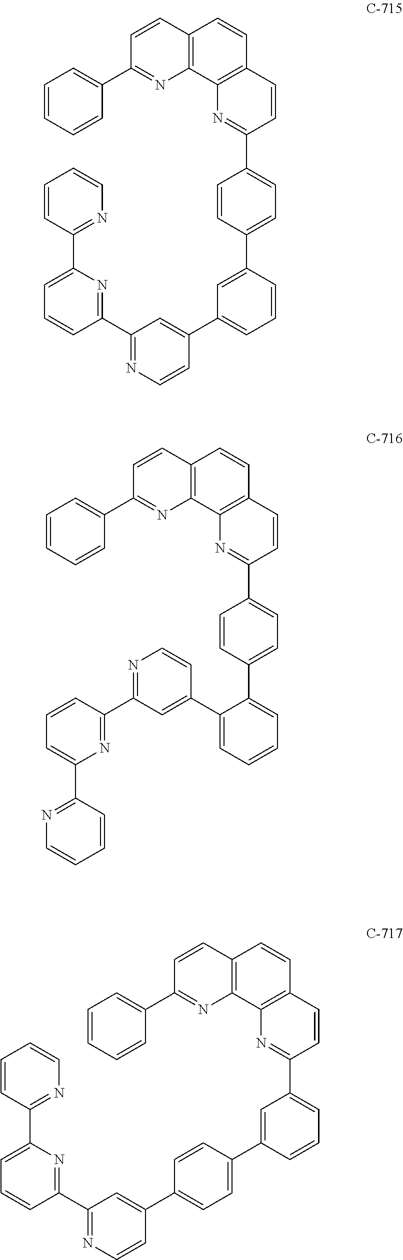 [SMILES: c1ccc(-c2ccc3ccc4ccc(-c5ccc(-c6cccc(-c7ccnc(-c8cccc(-c9ccccn9)n8)c7)c6)cc5)nc4c3n2)cc1 ; c1ccc(-c2ccc3ccc4ccc(-c5ccc(-c6ccccc6-c6ccnc(-c7cccc(-c8ccccn8)n7)c6)cc5)nc4c3n2)cc1 ; c1ccc(-c2ccc3ccc4ccc(-c5cccc(-c6ccc(-c7ccnc(-c8cccc(-c9ccccn9)n8)c7)cc6)c5)nc4c3n2)cc1]

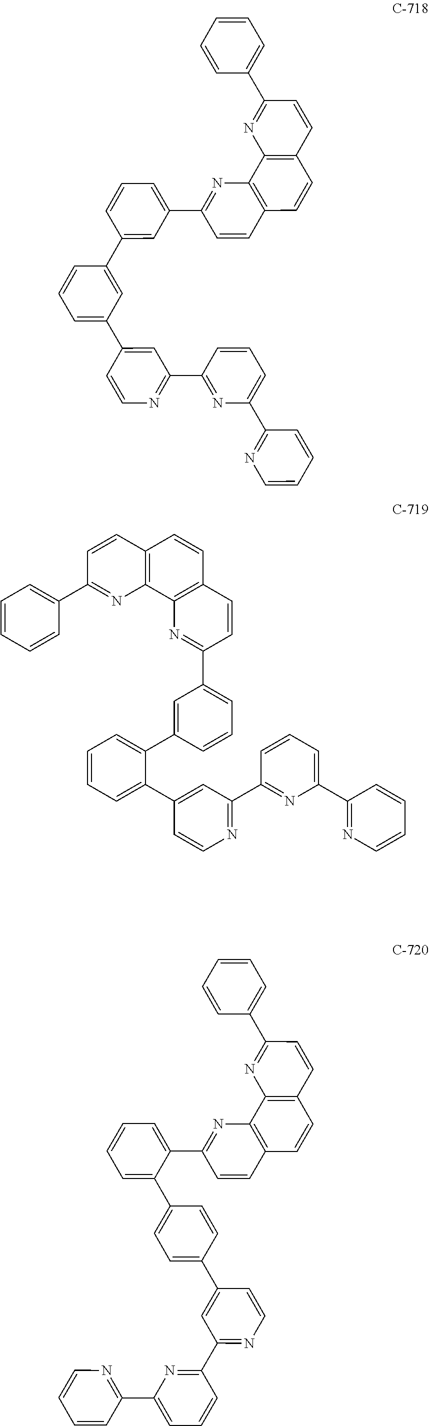 [SMILES: c1ccc(-c2ccc3ccc4ccc(-c5cccc(-c6cccc(-c7ccnc(-c8cccc(-c9ccccn9)n8)c7)c6)c5)nc4c3n2)cc1 ; c1ccc(-c2ccc3ccc4ccc(-c5cccc(-c6ccccc6-c6ccnc(-c7cccc(-c8ccccn8)n7)c6)c5)nc4c3n2)cc1 ; c1ccc(-c2ccc3ccc4ccc(-c5ccccc5-c5ccc(-c6ccnc(-c7cccc(-c8ccccn8)n7)c6)cc5)nc4c3n2)cc1]

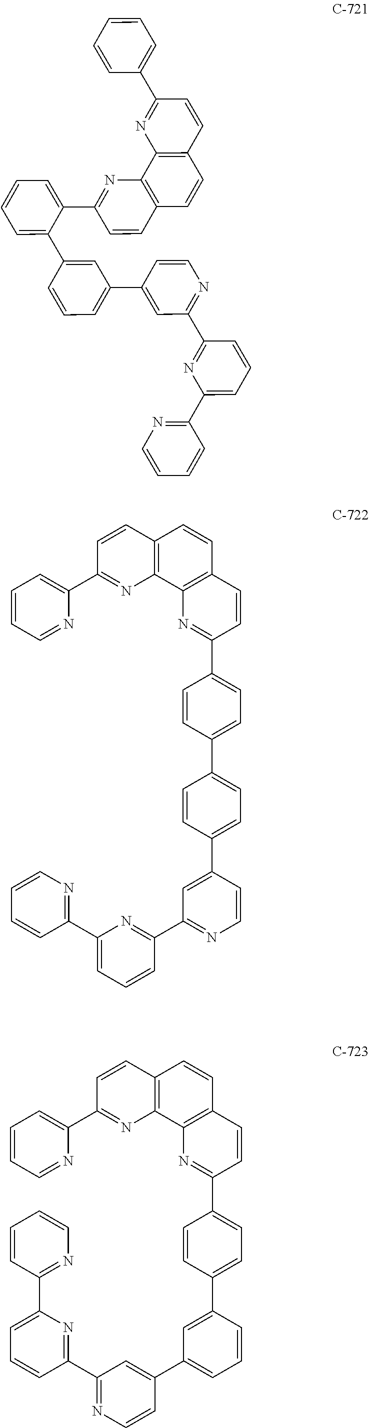 [SMILES: c1ccc(-c2ccc3ccc4ccc(-c5ccccc5-c5cccc(-c6ccnc(-c7cccc(-c8ccccn8)n7)c6)c5)nc4c3n2)cc1 ; c1ccc(-c2cccc(-c3cc(-c4ccc(-c5ccc(-c6ccc7ccc8ccc(-c9ccccn9)nc8c7n6)cc5)cc4)ccn3)n2)nc1 ; c1ccc(-c2cccc(-c3cc(-c4cccc(-c5ccc(-c6ccc7ccc8ccc(-c9ccccn9)nc8c7n6)cc5)c4)ccn3)n2)nc1]

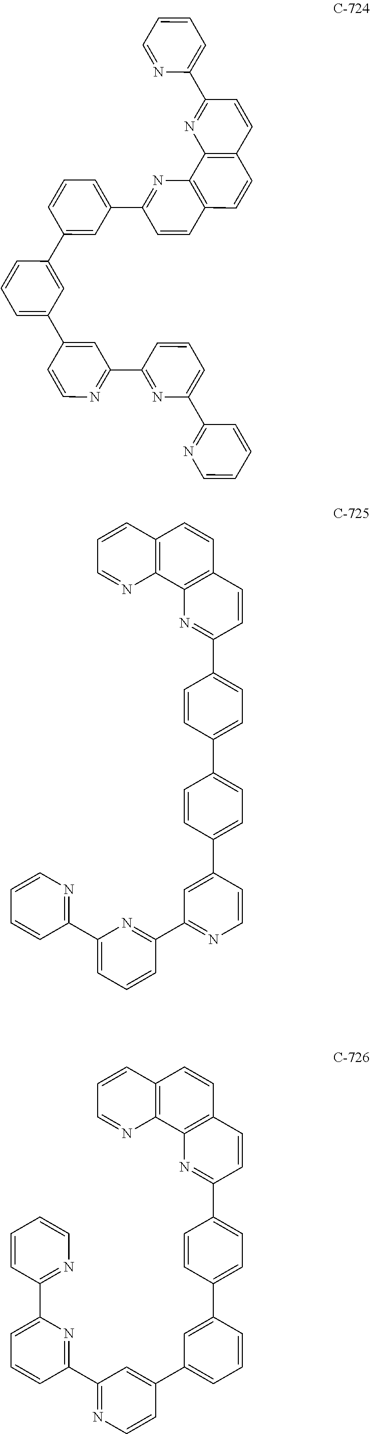 [SMILES: c1ccc(-c2cccc(-c3cc(-c4cccc(-c5cccc(-c6ccc7ccc8ccc(-c9ccccn9)nc8c7n6)c5)c4)ccn3)n2)nc1 ; c1ccc(-c2cccc(-c3cc(-c4ccc(-c5ccc(-c6ccc7ccc8cccnc8c7n6)cc5)cc4)ccn3)n2)nc1 ; c1ccc(-c2cccc(-c3cc(-c4cccc(-c5ccc(-c6ccc7ccc8cccnc8c7n6)cc5)c4)ccn3)n2)nc1]

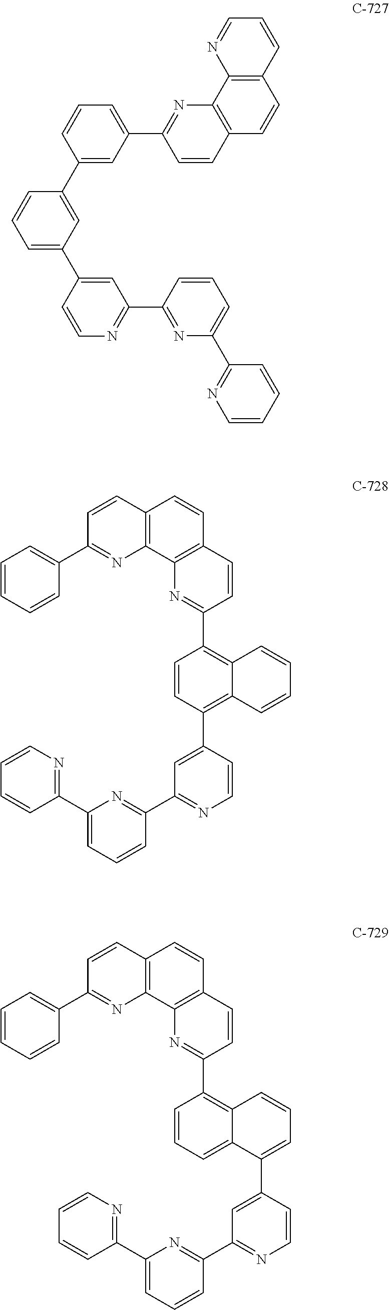 [SMILES: c1ccc(-c2cccc(-c3cc(-c4cccc(-c5cccc(-c6ccc7ccc8cccnc8c7n6)c5)c4)ccn3)n2)nc1 ; c1ccc(-c2ccc3ccc4ccc(-c5ccc(-c6ccnc(-c7cccc(-c8ccccn8)n7)c6)c6ccccc56)nc4c3n2)cc1 ; c1ccc(-c2ccc3ccc4ccc(-c5cccc6c(-c7ccnc(-c8cccc(-c9ccccn9)n8)c7)cccc56)nc4c3n2)cc1]

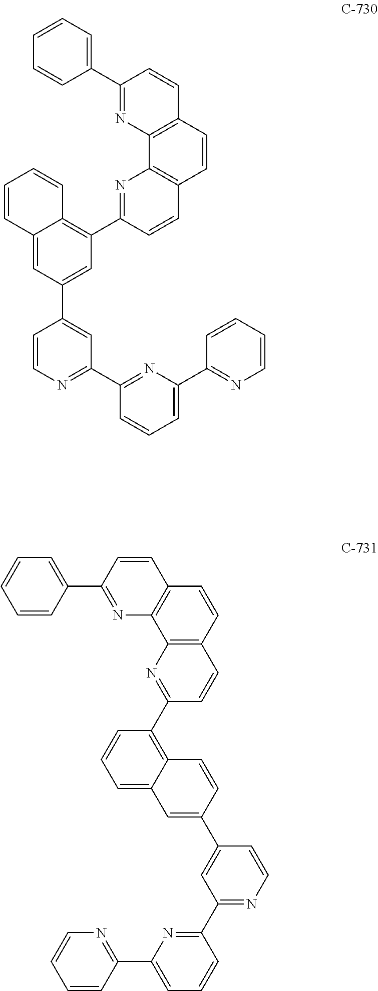 [SMILES: c1ccc(-c2ccc3ccc4ccc(-c5cc(-c6ccnc(-c7cccc(-c8ccccn8)n7)c6)cc6ccccc56)nc4c3n2)cc1 ; c1ccc(-c2ccc3ccc4ccc(-c5cccc6cc(-c7ccnc(-c8cccc(-c9ccccn9)n8)c7)ccc56)nc4c3n2)cc1]

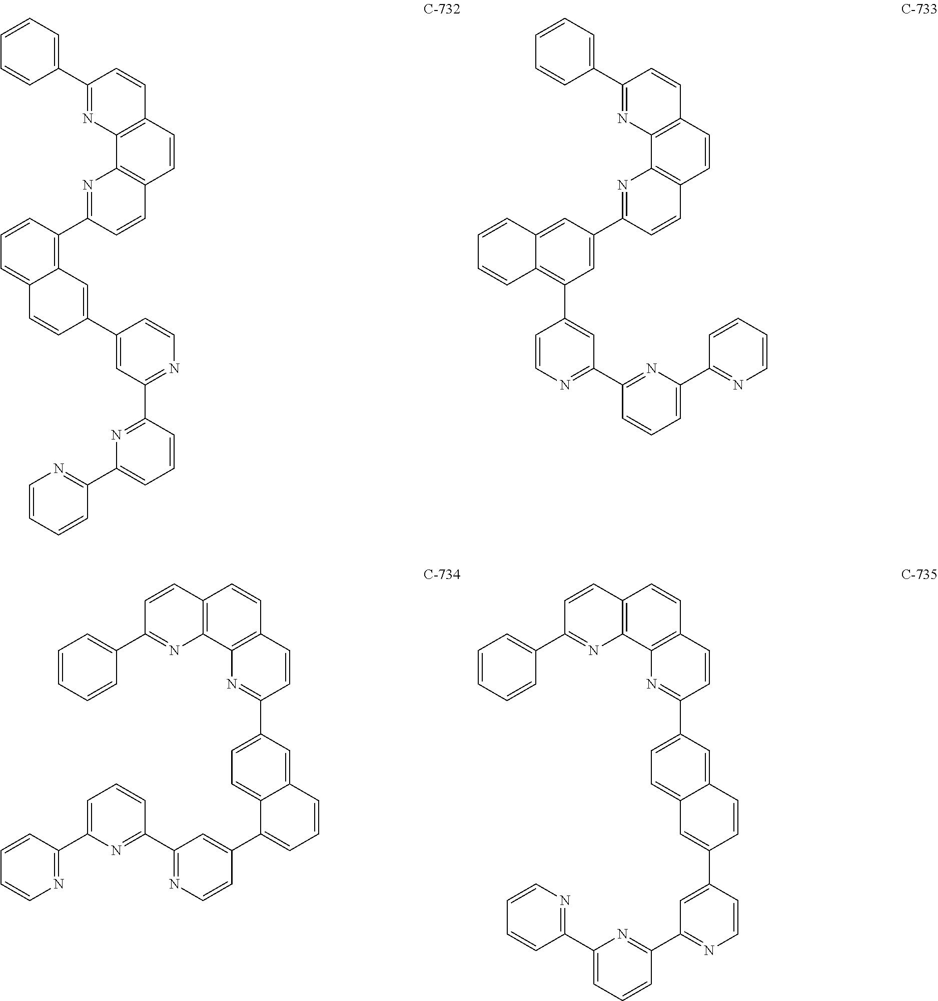 [SMILES: c1ccc(-c2ccc3ccc4ccc(-c5cccc6ccc(-c7ccnc(-c8cccc(-c9ccccn9)n8)c7)cc56)nc4c3n2)cc1 ; c1ccc(-c2ccc3ccc4ccc(-c5cc(-c6ccnc(-c7cccc(-c8ccccn8)n7)c6)c6ccccc6c5)nc4c3n2)cc1 ; c1ccc(-c2ccc3ccc4ccc(-c5ccc6c(-c7ccnc(-c8cccc(-c9ccccn9)n8)c7)cccc6c5)nc4c3n2)cc1 ; c1ccc(-c2ccc3ccc4ccc(-c5ccc6cc(-c7ccnc(-c8cccc(-c9ccccn9)n8)c7)ccc6c5)nc4c3n2)cc1]

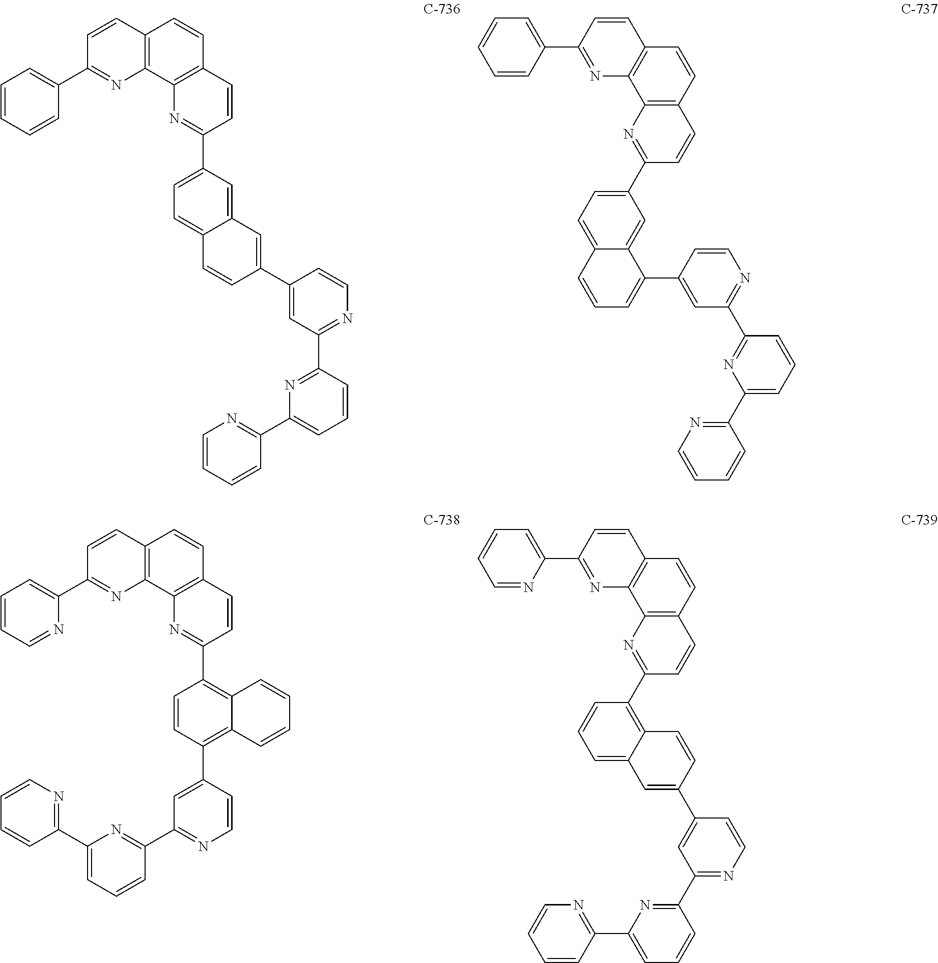 [SMILES: c1ccc(-c2ccc3ccc4ccc(-c5ccc6ccc(-c7ccnc(-c8cccc(-c9ccccn9)n8)c7)cc6c5)nc4c3n2)cc1 ; c1ccc(-c2ccc3ccc4ccc(-c5ccc6cccc(-c7ccnc(-c8cccc(-c9ccccn9)n8)c7)c6c5)nc4c3n2)cc1 ; c1ccc(-c2cccc(-c3cc(-c4ccc(-c5ccc6ccc7ccc(-c8ccccn8)nc7c6n5)c5ccccc45)ccn3)n2)nc1 ; c1ccc(-c2cccc(-c3cc(-c4ccc5c(-c6ccc7ccc8ccc(-c9ccccn9)nc8c7n6)cccc5c4)ccn3)n2)nc1]

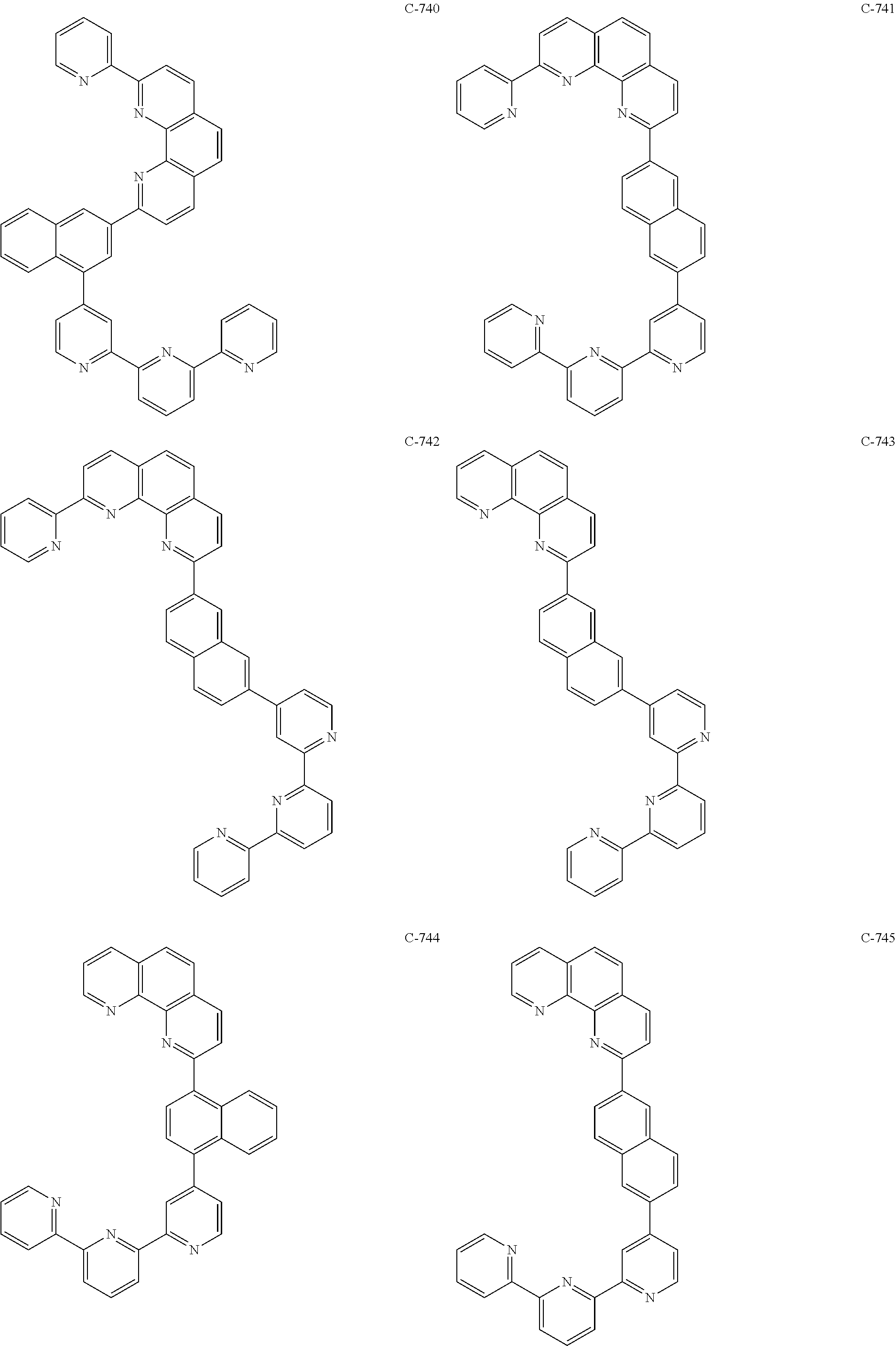

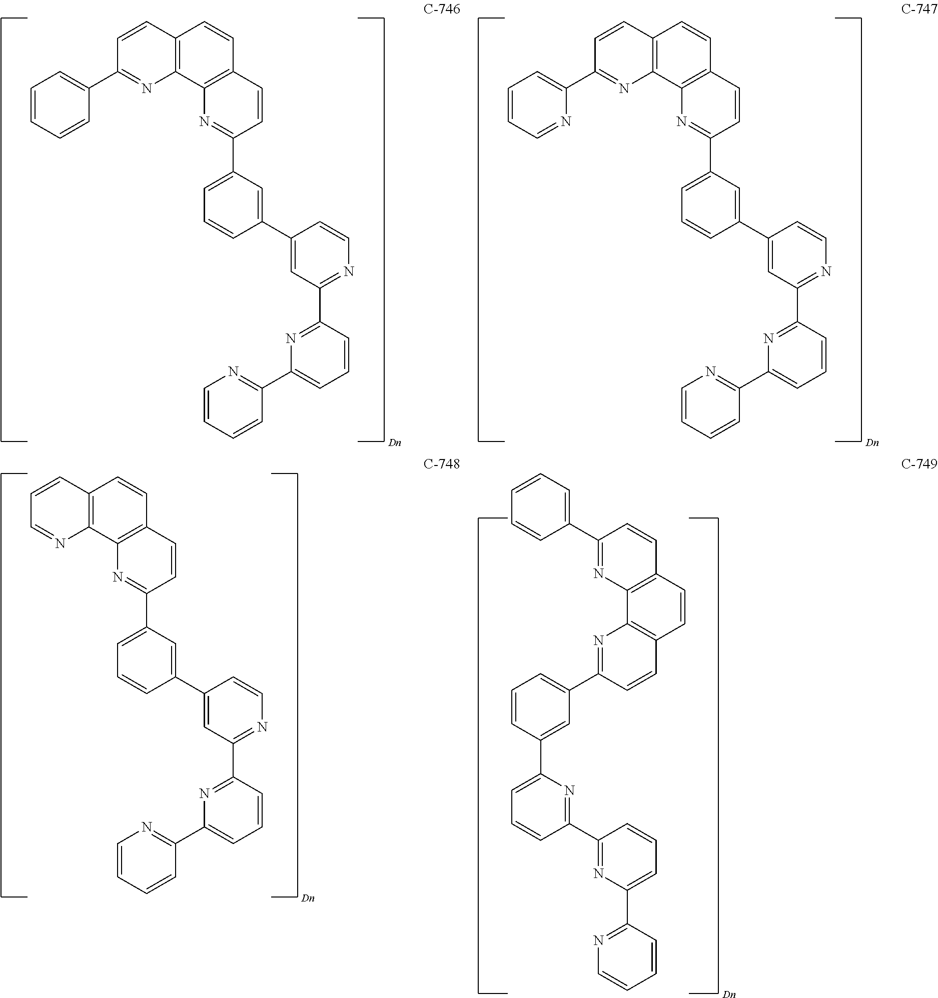

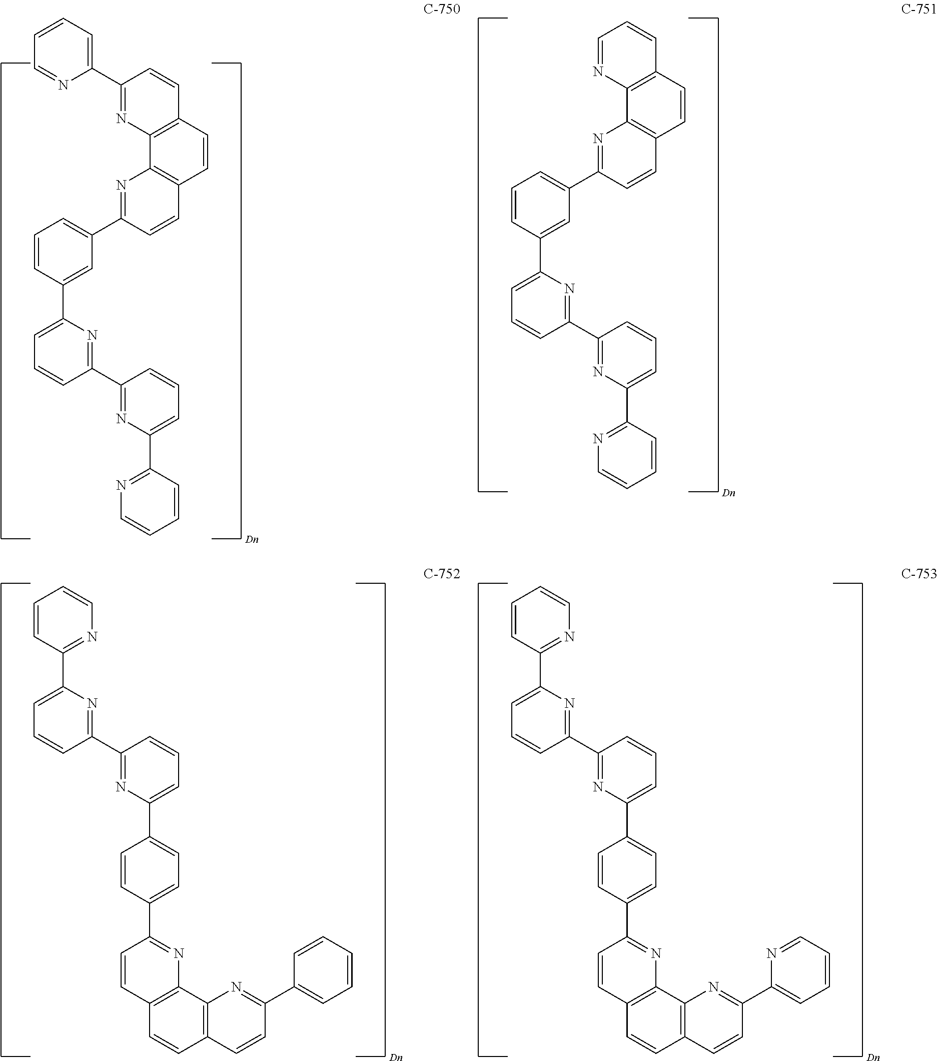

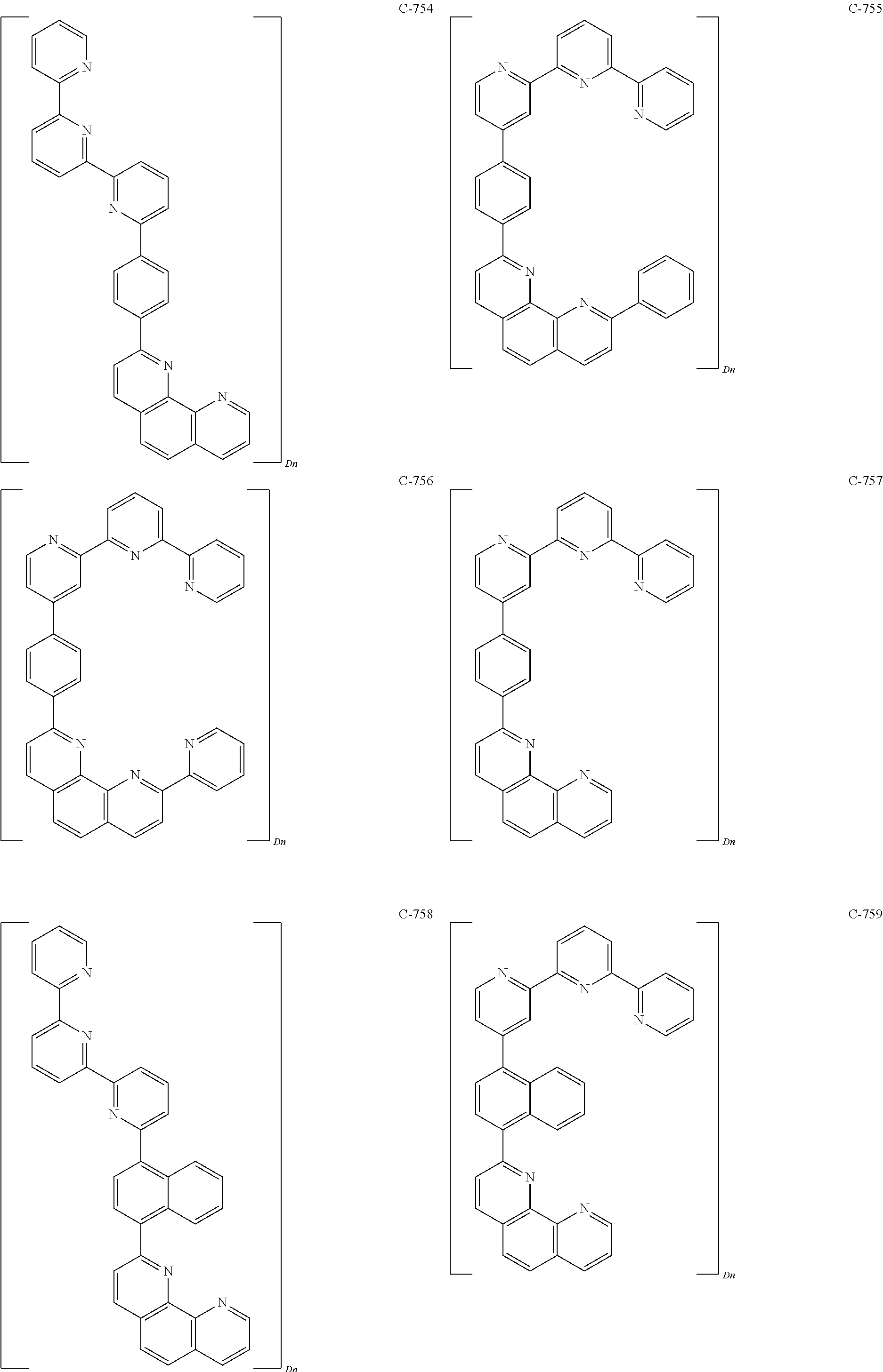

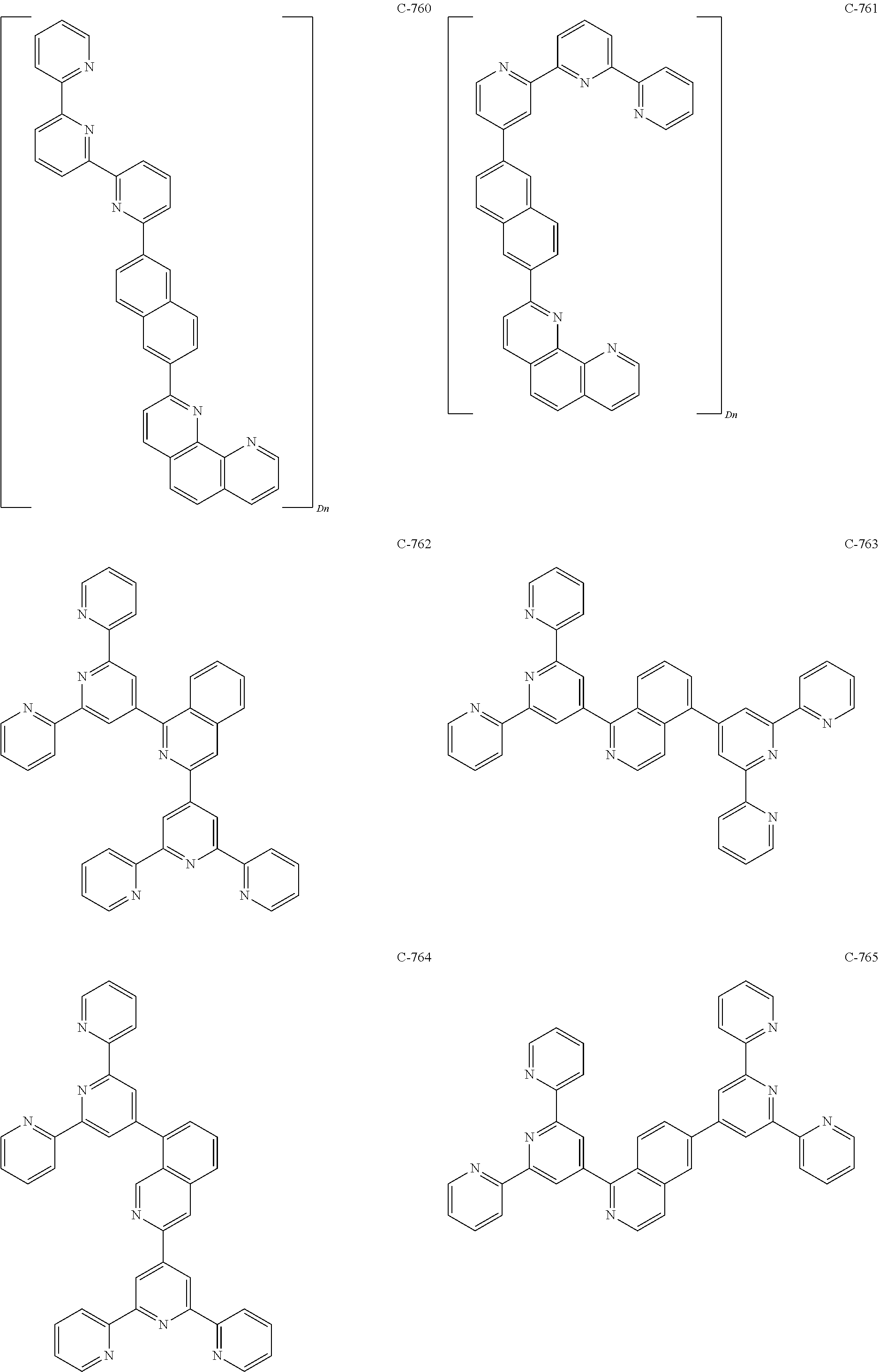

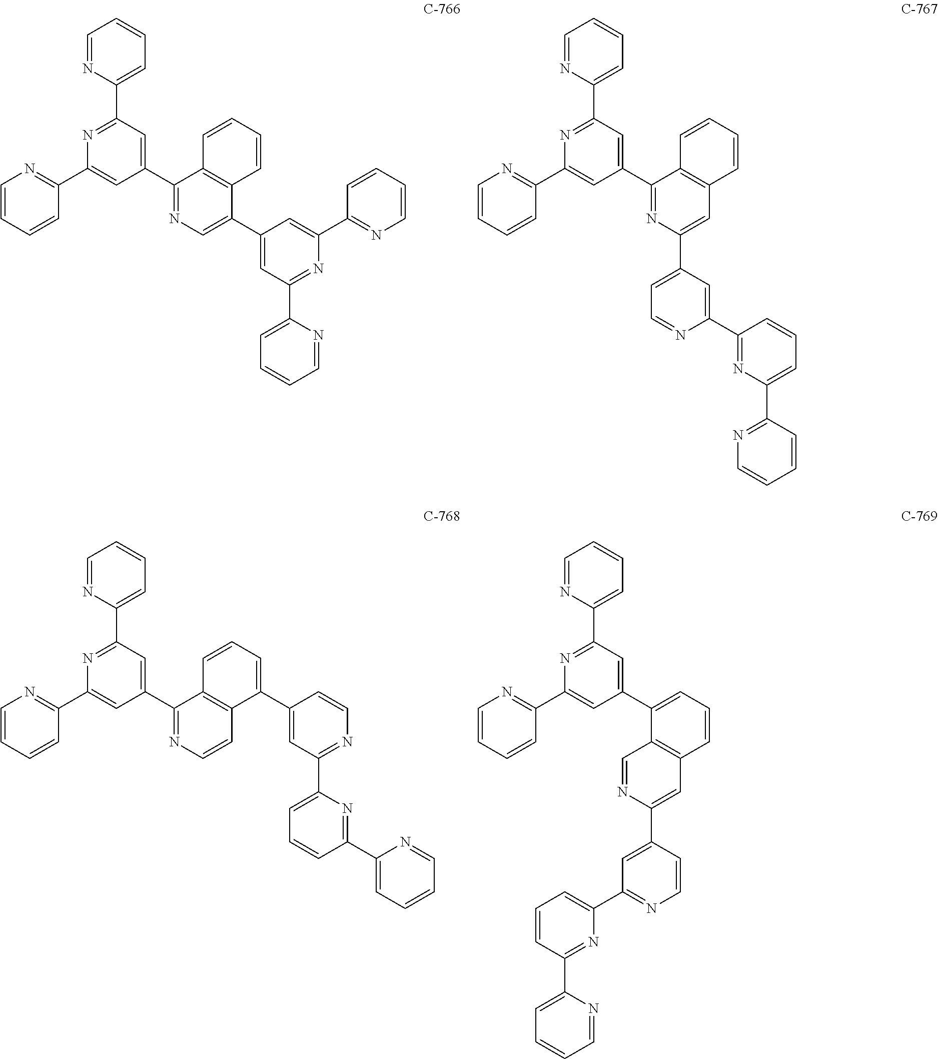 [SMILES: c1ccc(-c2cc(-c3cnc(-c4cc(-c5ccccn5)nc(-c5ccccn5)c4)c4ccccc34)cc(-c3ccccn3)n2)nc1 ; c1ccc(-c2cccc(-c3cc(-c4cc5ccccc5c(-c5cc(-c6ccccn6)nc(-c6ccccn6)c5)n4)ccn3)n2)nc1 ; c1ccc(-c2cccc(-c3cc(-c4cccc5c(-c6cc(-c7ccccn7)nc(-c7ccccn7)c6)nccc45)ccn3)n2)nc1 ; c1ccc(-c2cccc(-c3cc(-c4cc5cccc(-c6cc(-c7ccccn7)nc(-c7ccccn7)c6)c5cn4)ccn3)n2)nc1]

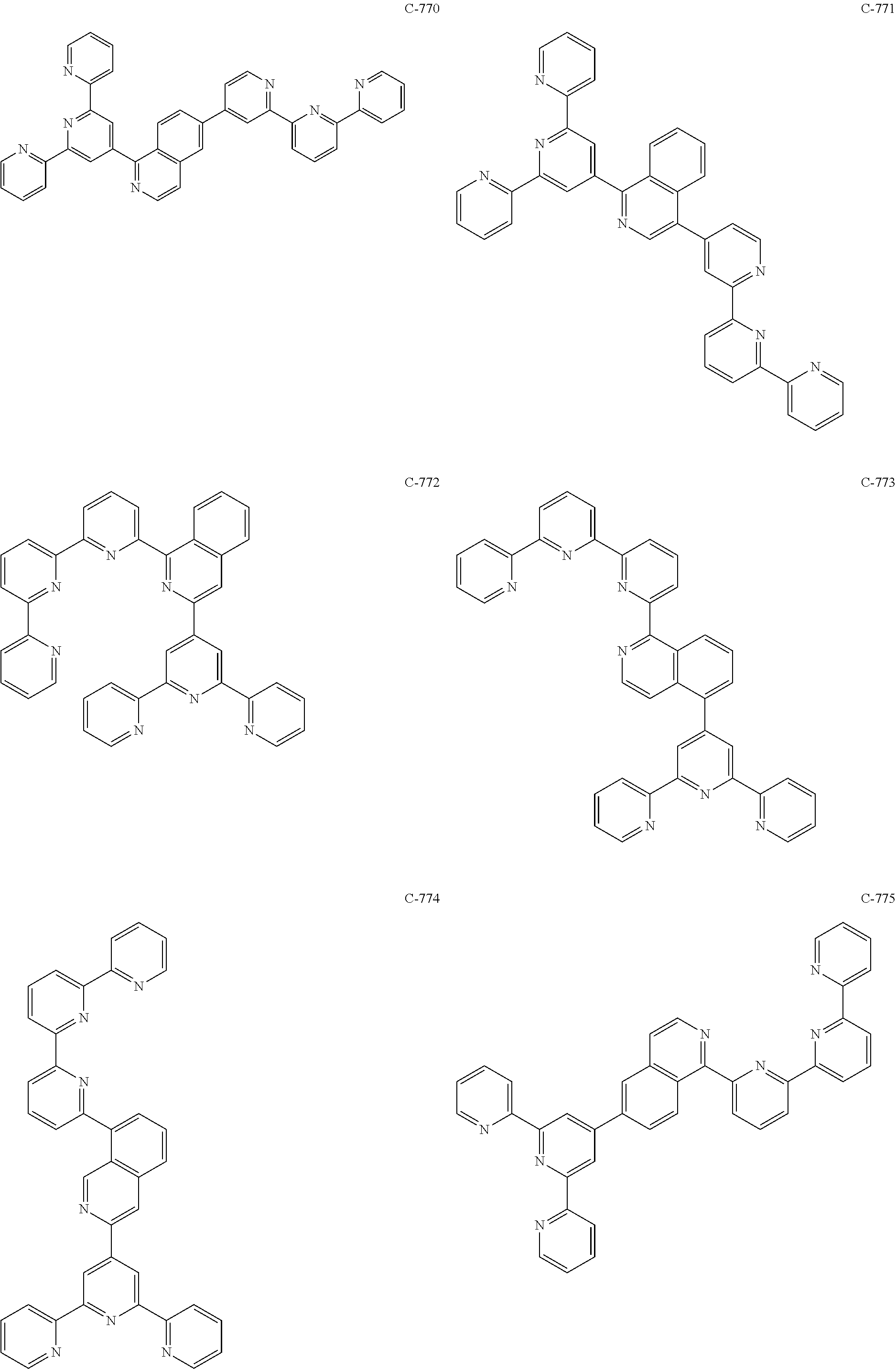 [SMILES: c1ccc(-c2cccc(-c3cc(-c4ccc5c(-c6cc(-c7ccccn7)nc(-c7ccccn7)c6)nccc5c4)ccn3)n2)nc1 ; c1ccc(-c2cccc(-c3cc(-c4cnc(-c5cc(-c6ccccn6)nc(-c6ccccn6)c5)c5ccccc45)ccn3)n2)nc1 ; c1ccc(-c2cc(-c3cc4ccccc4c(-c4cccc(-c5cccc(-c6ccccn6)n5)n4)n3)cc(-c3ccccn3)n2)nc1 ; c1ccc(-c2cc(-c3cccc4c(-c5cccc(-c6cccc(-c7ccccn7)n6)n5)nccc34)cc(-c3ccccn3)n2)nc1 ; c1ccc(-c2cc(-c3cc4cccc(-c5cccc(-c6cccc(-c7ccccn7)n6)n5)c4cn3)cc(-c3ccccn3)n2)nc1 ; c1ccc(-c2cc(-c3ccc4c(-c5cccc(-c6cccc(-c7ccccn7)n6)n5)nccc4c3)cc(-c3ccccn3)n2)nc1]

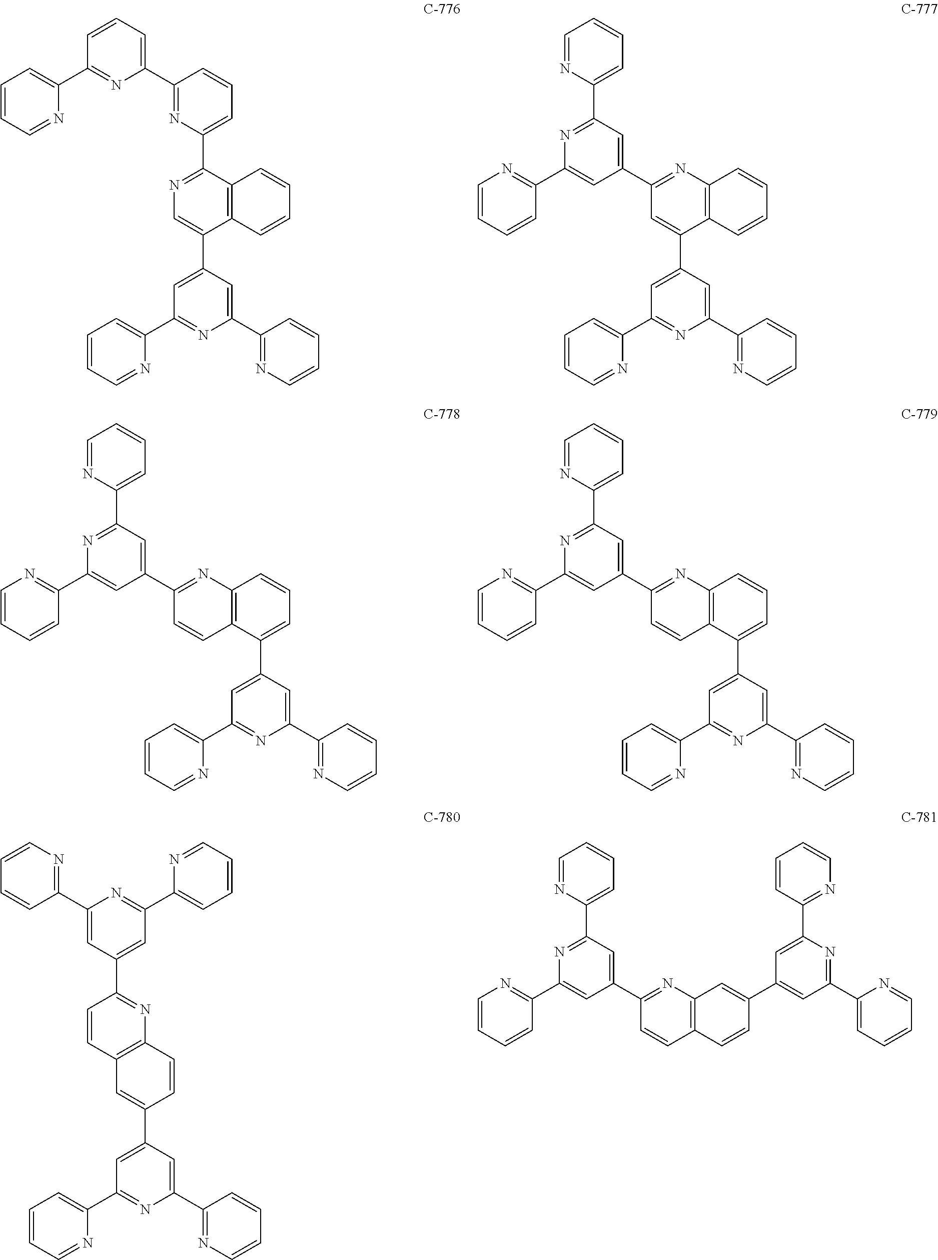

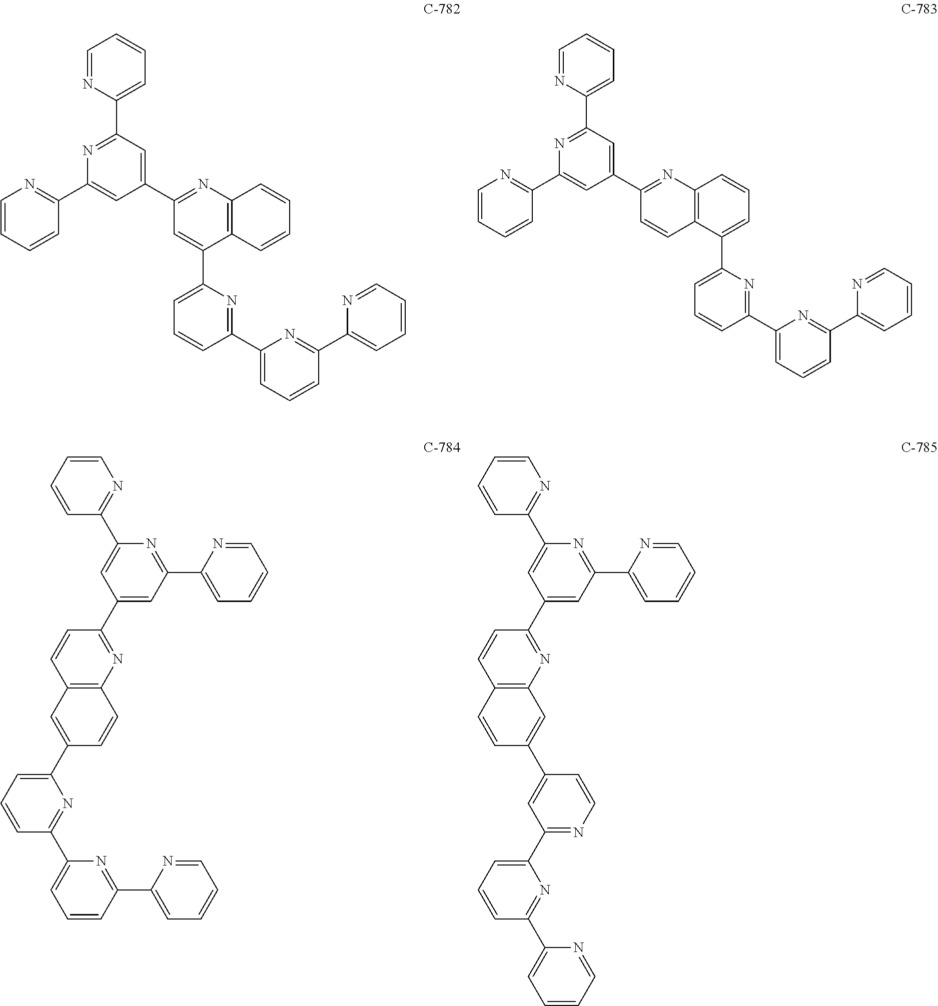 [SMILES: c1ccc(-c2cc(-c3cc(-c4cccc(-c5cccc(-c6ccccn6)n5)n4)c4ccccc4n3)cc(-c3ccccn3)n2)nc1 ; c1ccc(-c2cc(-c3ccc4c(-c5cccc(-c6cccc(-c7ccccn7)n6)n5)cccc4n3)cc(-c3ccccn3)n2)nc1 ; c1ccc(-c2cc(-c3ccc4cc(-c5cccc(-c6cccc(-c7ccccn7)n6)n5)ccc4n3)cc(-c3ccccn3)n2)nc1 ; c1ccc(-c2cccc(-c3cc(-c4ccc5ccc(-c6cc(-c7ccccn7)nc(-c7ccccn7)c6)nc5c4)ccn3)n2)nc1]

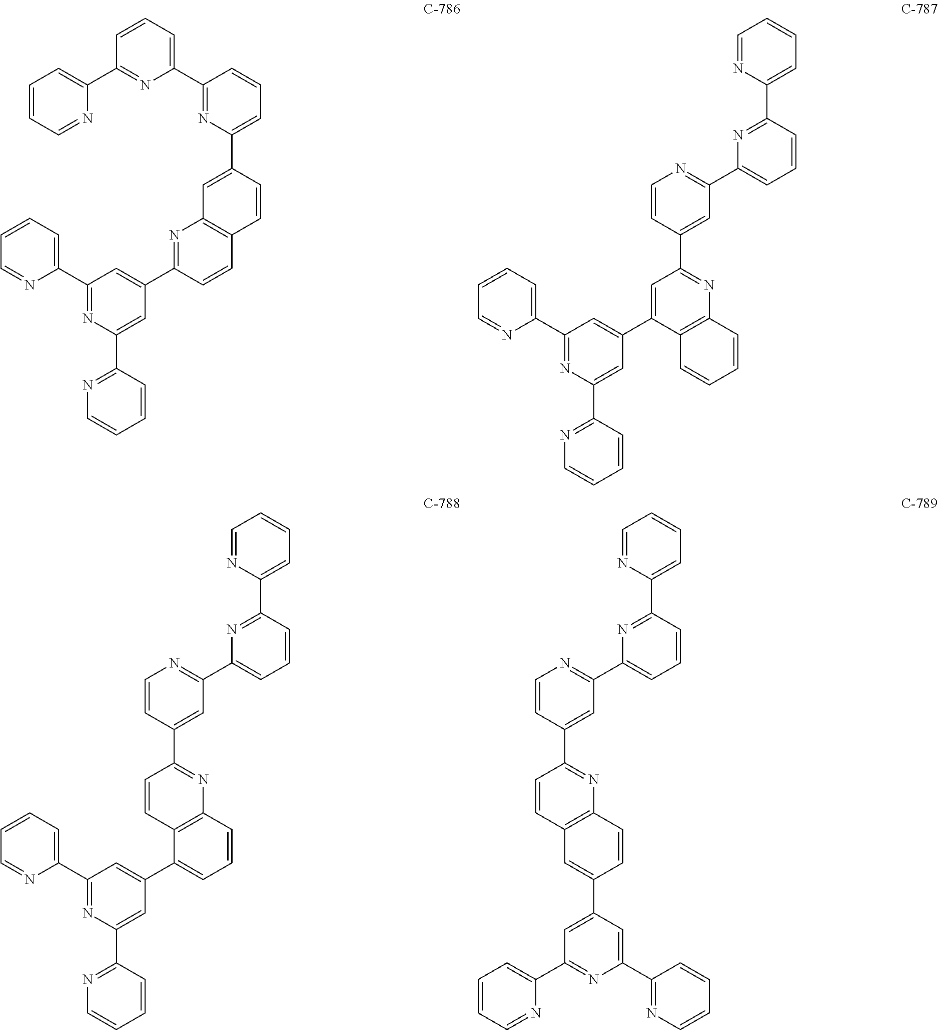 [SMILES: c1ccc(-c2cc(-c3ccc4ccc(-c5cccc(-c6cccc(-c7ccccn7)n6)n5)cc4n3)cc(-c3ccccn3)n2)nc1 ; c1ccc(-c2cccc(-c3cc(-c4cc(-c5cc(-c6ccccn6)nc(-c6ccccn6)c5)c5ccccc5n4)ccn3)n2)nc1 ; c1ccc(-c2cccc(-c3cc(-c4ccc5c(-c6cc(-c7ccccn7)nc(-c7ccccn7)c6)cccc5n4)ccn3)n2)nc1 ; c1ccc(-c2cccc(-c3cc(-c4ccc5cc(-c6cc(-c7ccccn7)nc(-c7ccccn7)c6)ccc5n4)ccn3)n2)nc1]

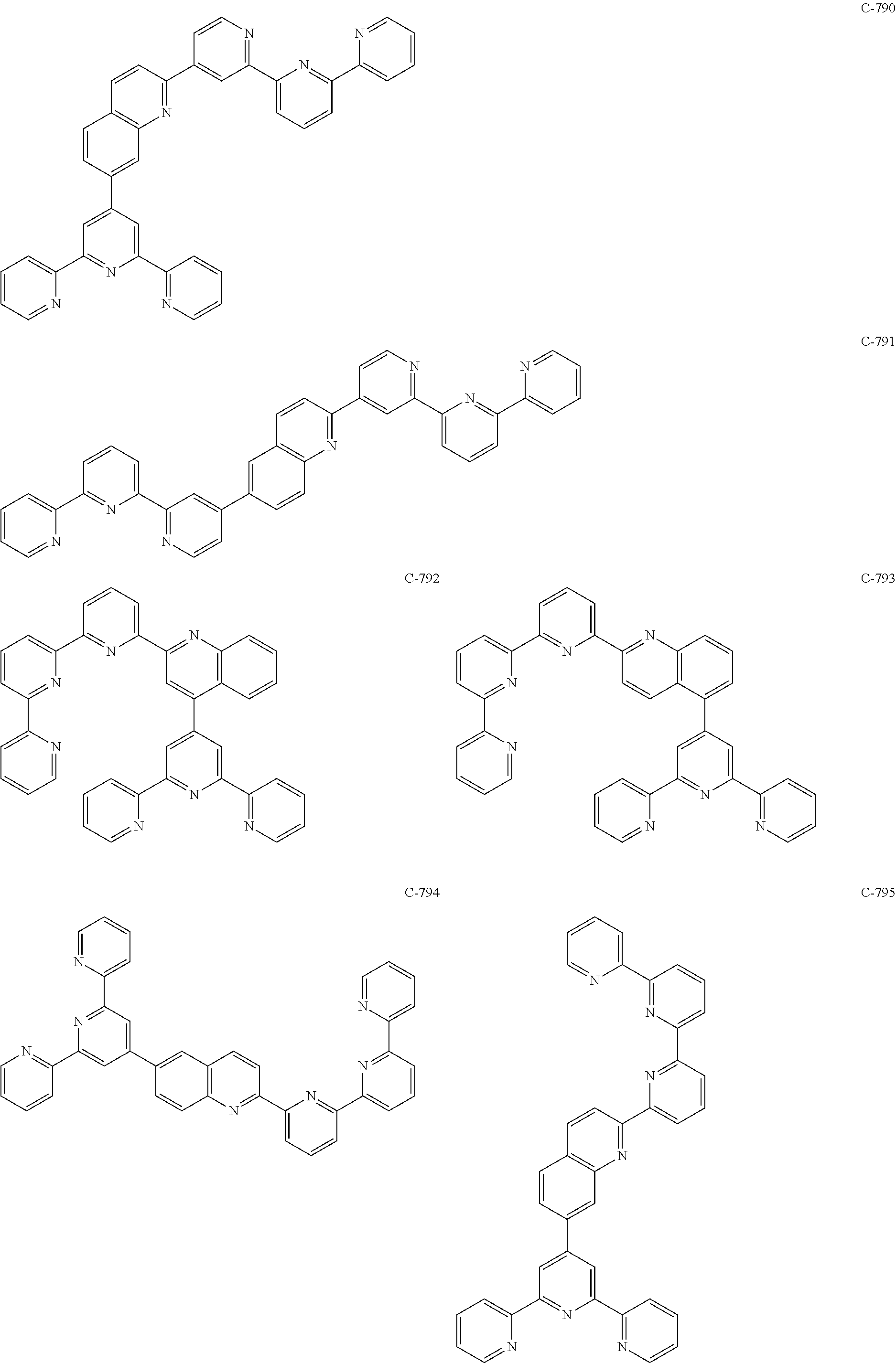 [SMILES: c1ccc(-c2cccc(-c3cc(-c4ccc5ccc(-c6cc(-c7ccccn7)nc(-c7ccccn7)c6)cc5n4)ccn3)n2)nc1 ; c1ccc(-c2cccc(-c3cc(-c4ccc5nc(-c6ccnc(-c7cccc(-c8ccccn8)n7)c6)ccc5c4)ccn3)n2)nc1 ; c1ccc(-c2cc(-c3cc(-c4cccc(-c5cccc(-c6ccccn6)n5)n4)nc4ccccc34)cc(-c3ccccn3)n2)nc1 ; c1ccc(-c2cc(-c3cccc4nc(-c5cccc(-c6cccc(-c7ccccn7)n6)n5)ccc34)cc(-c3ccccn3)n2)nc1 ; c1ccc(-c2cc(-c3ccc4nc(-c5cccc(-c6cccc(-c7ccccn7)n6)n5)ccc4c3)cc(-c3ccccn3)n2)nc1 ; c1ccc(-c2cc(-c3ccc4ccc(-c5cccc(-c6cccc(-c7ccccn7)n6)n5)nc4c3)cc(-c3ccccn3)n2)nc1]

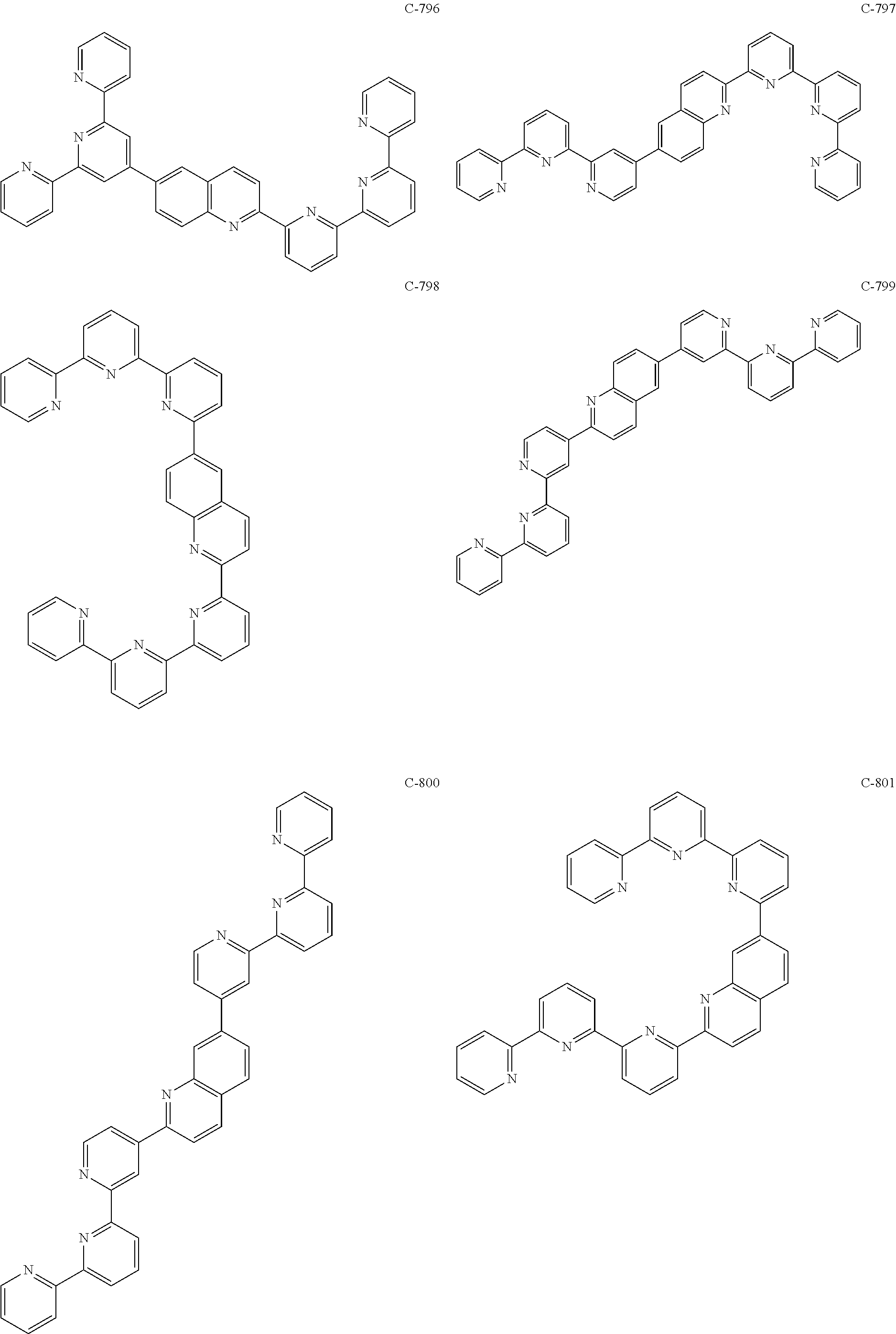

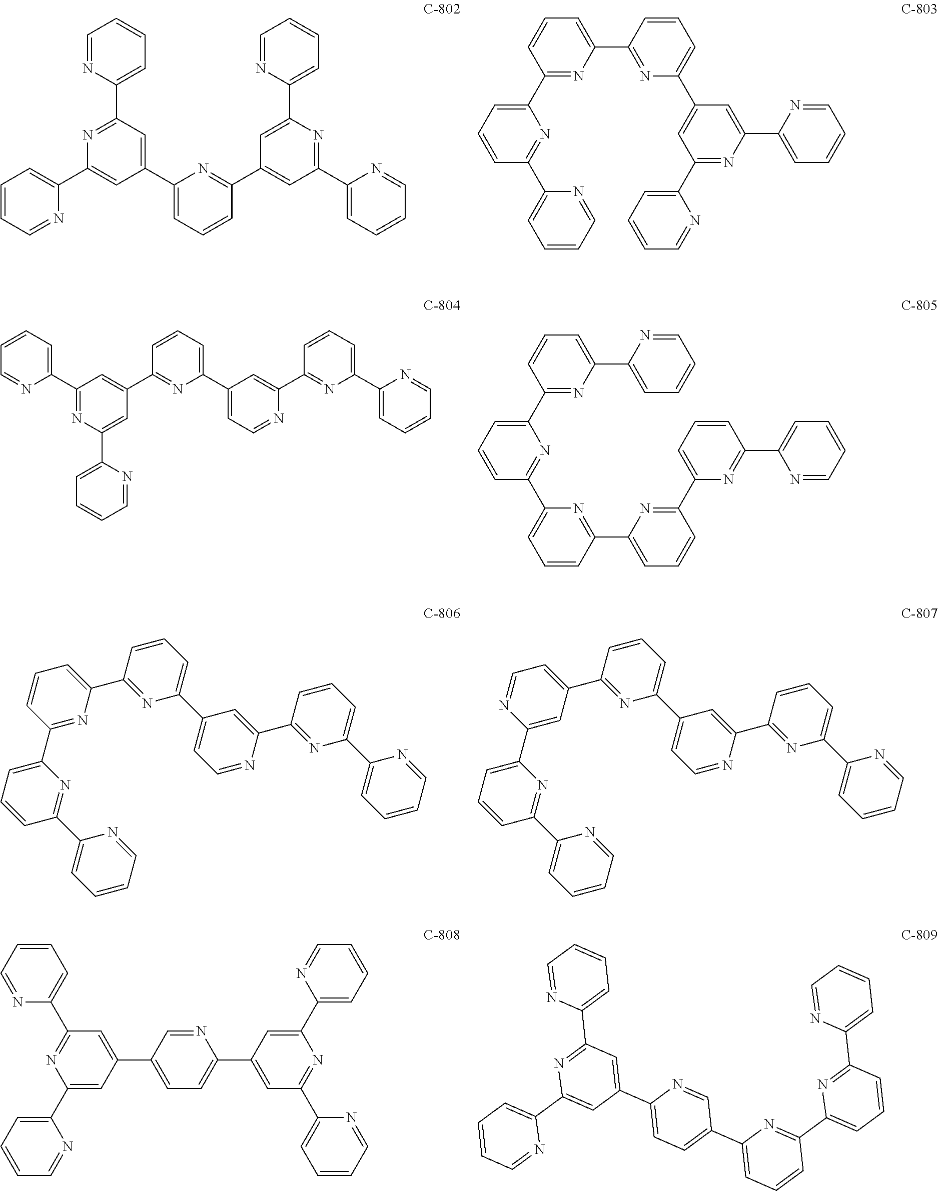

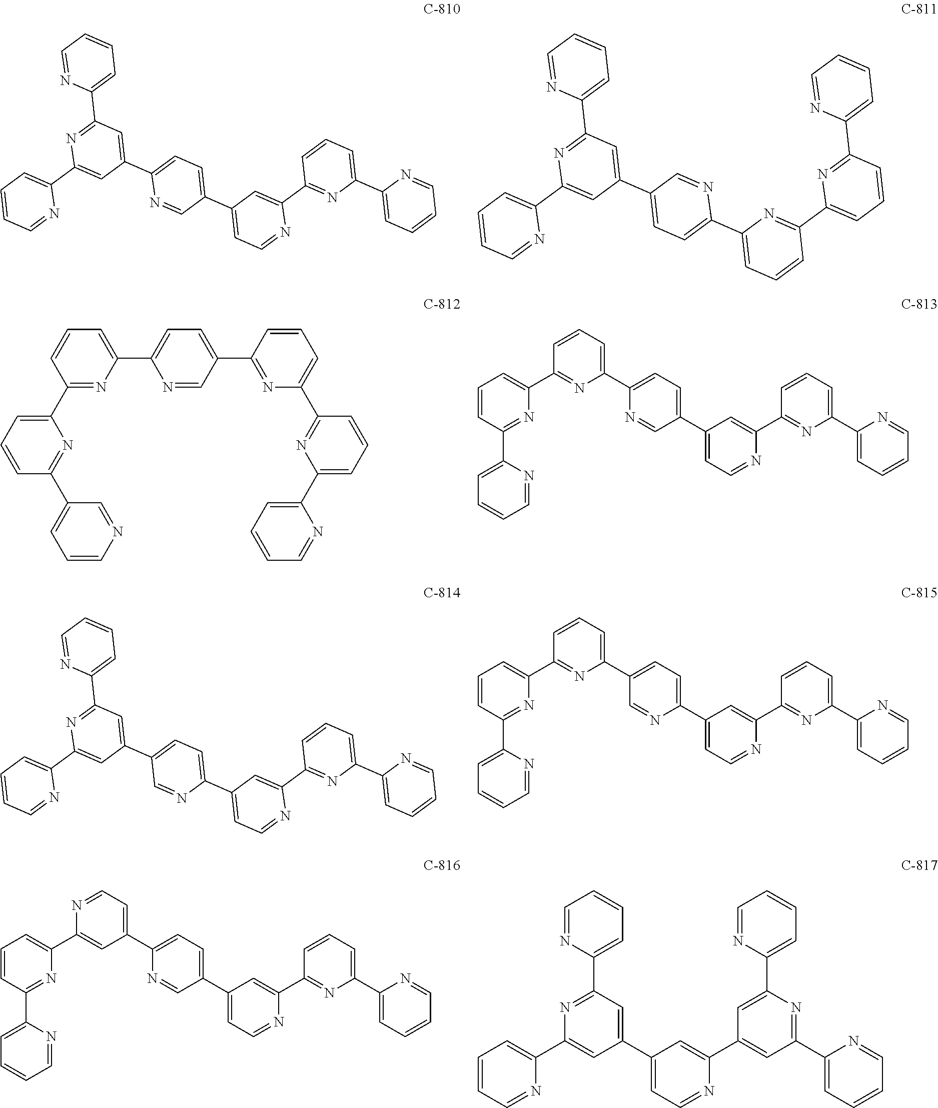

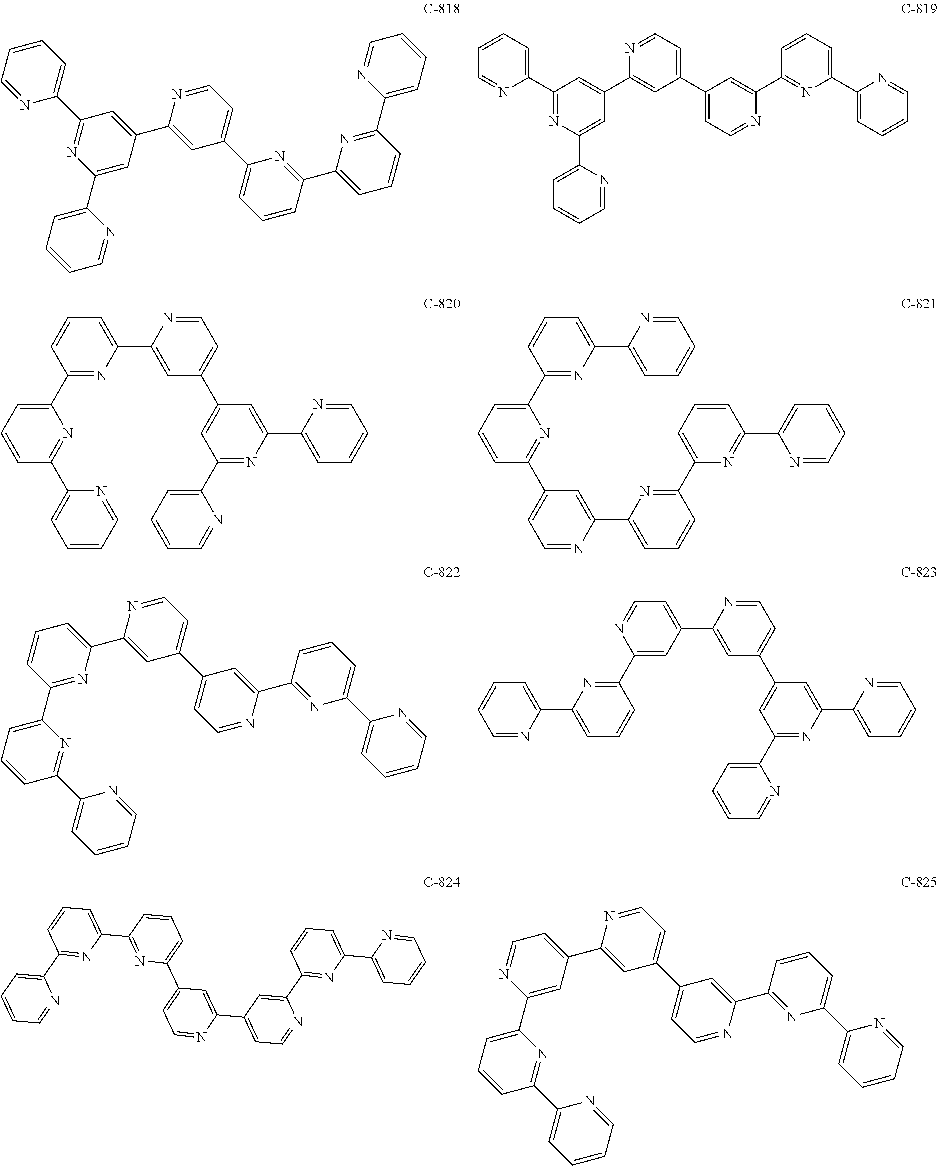

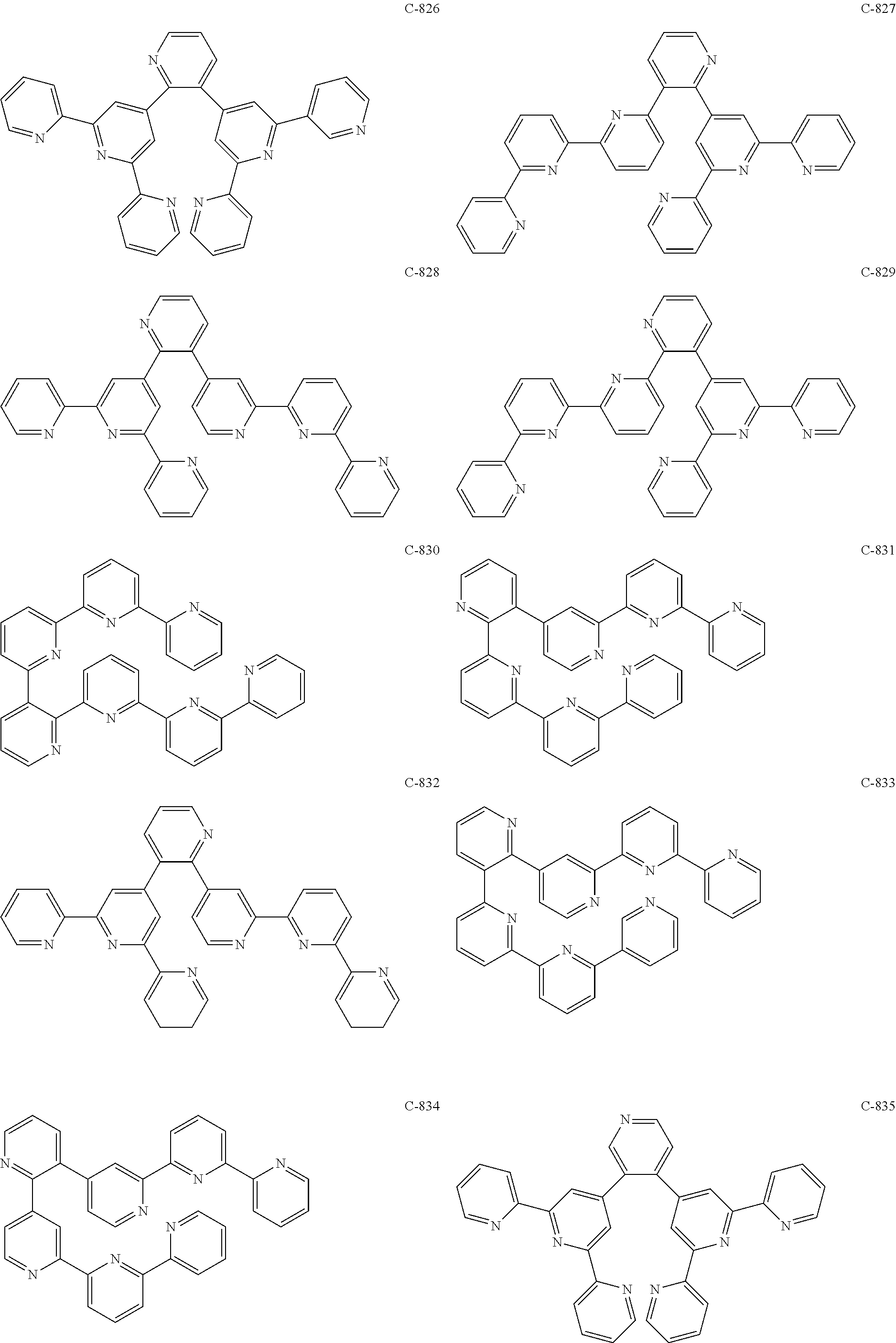

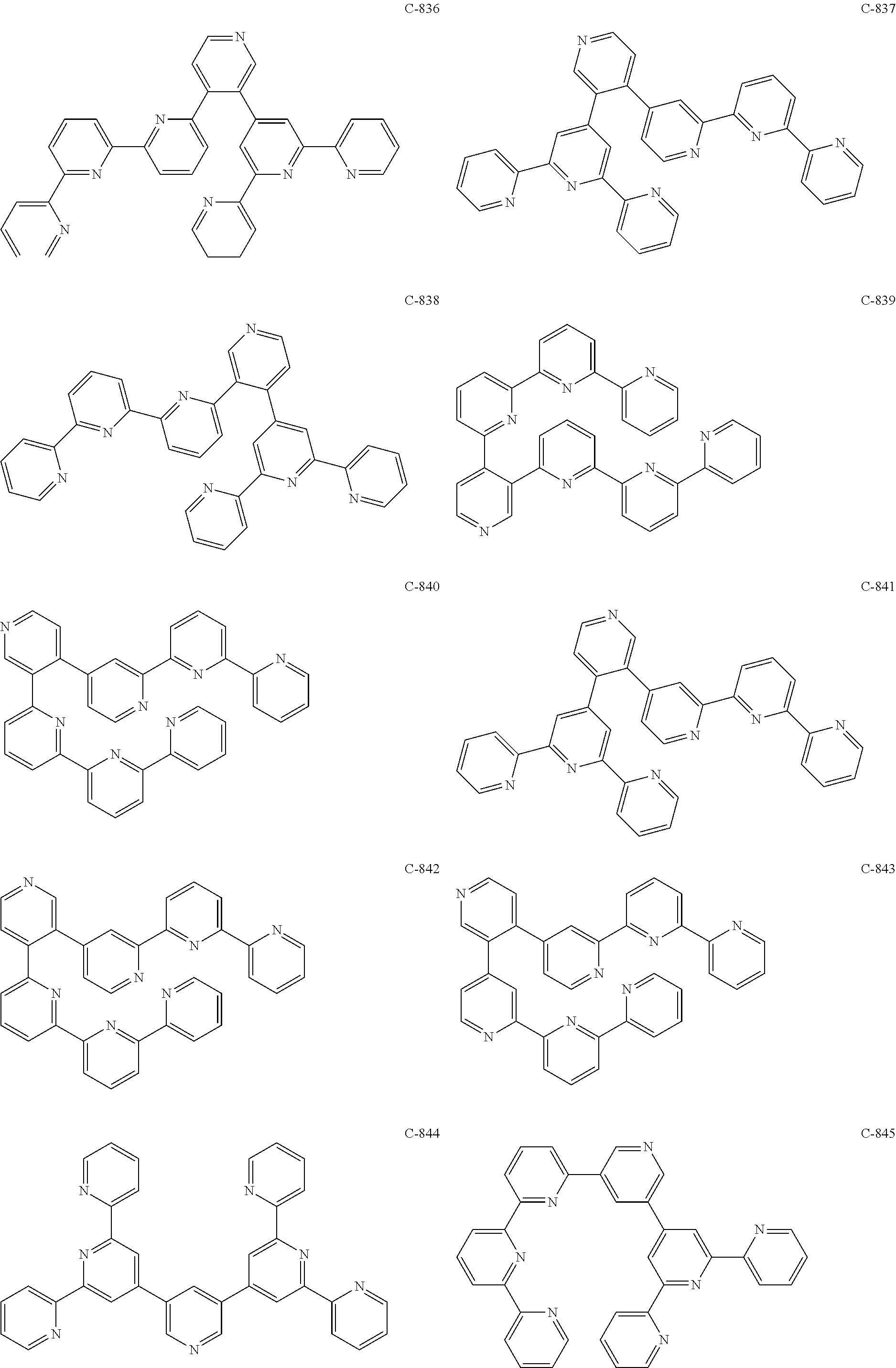

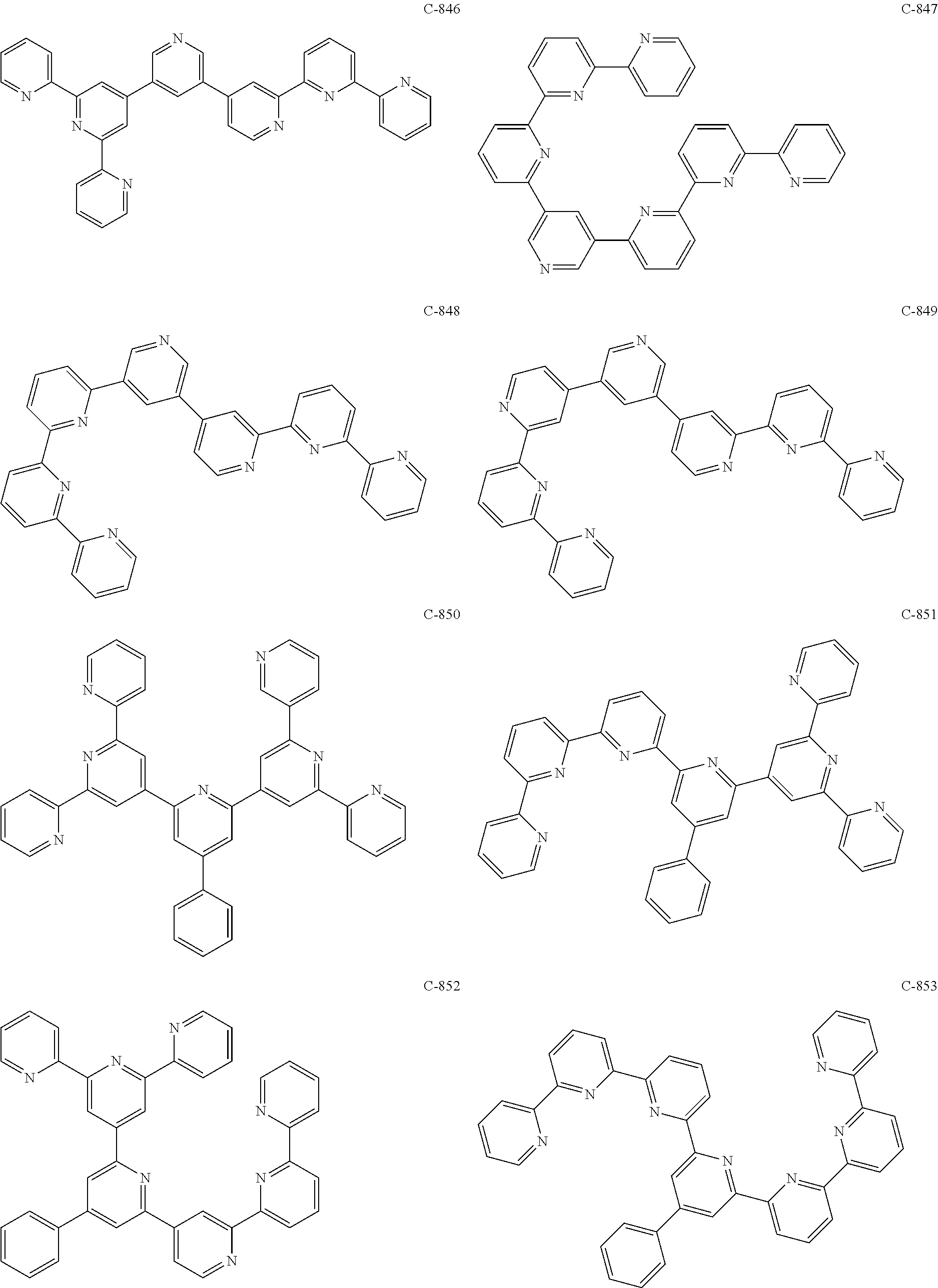

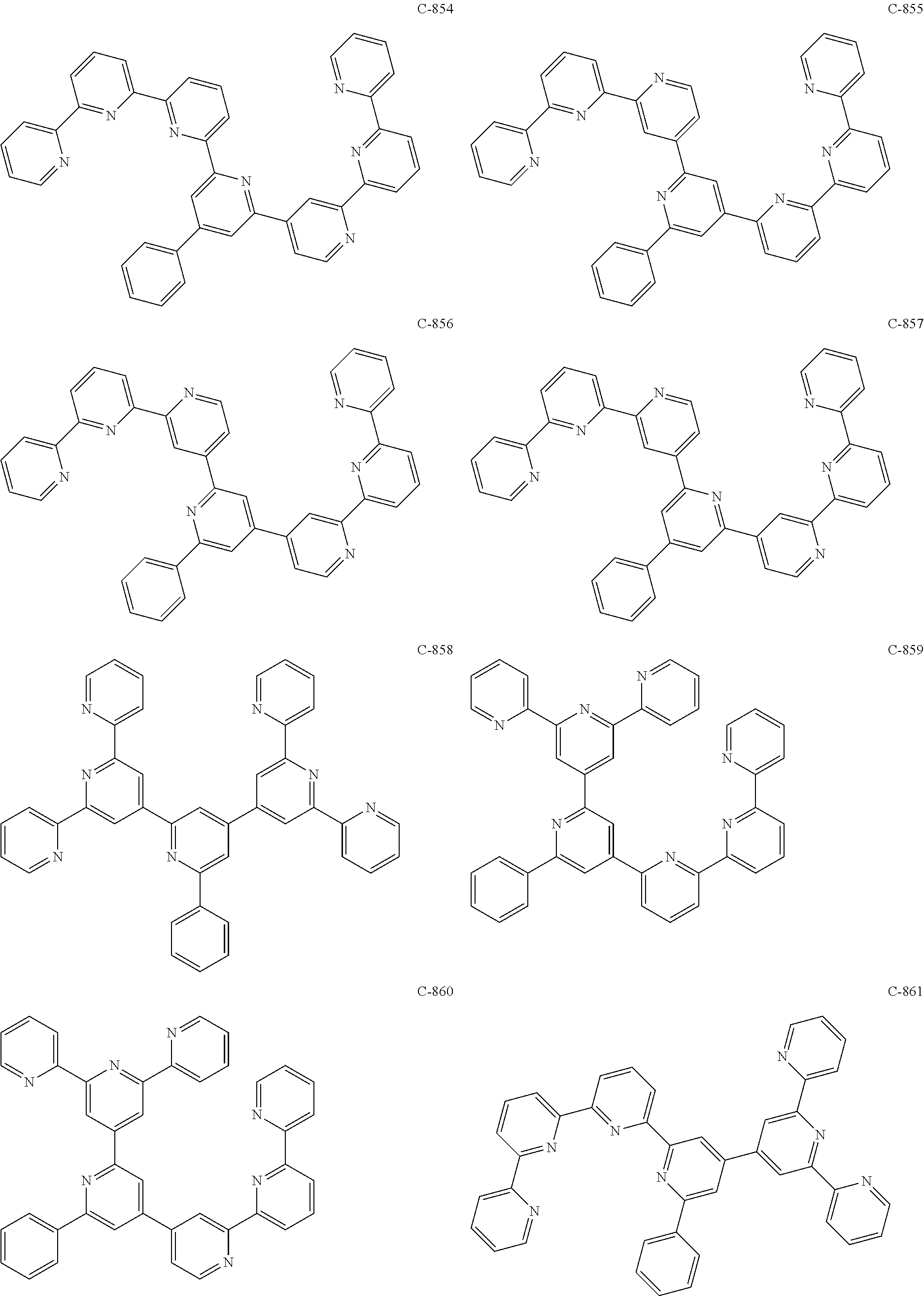

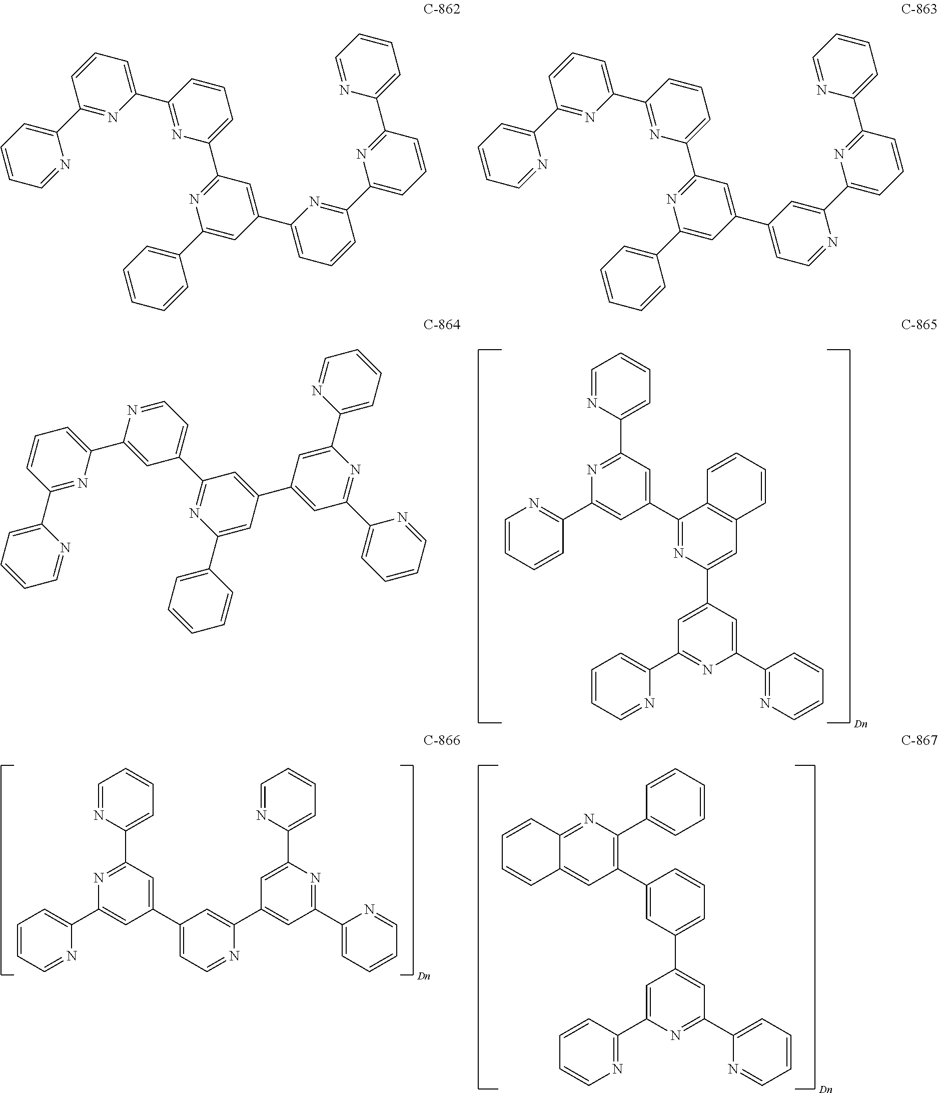

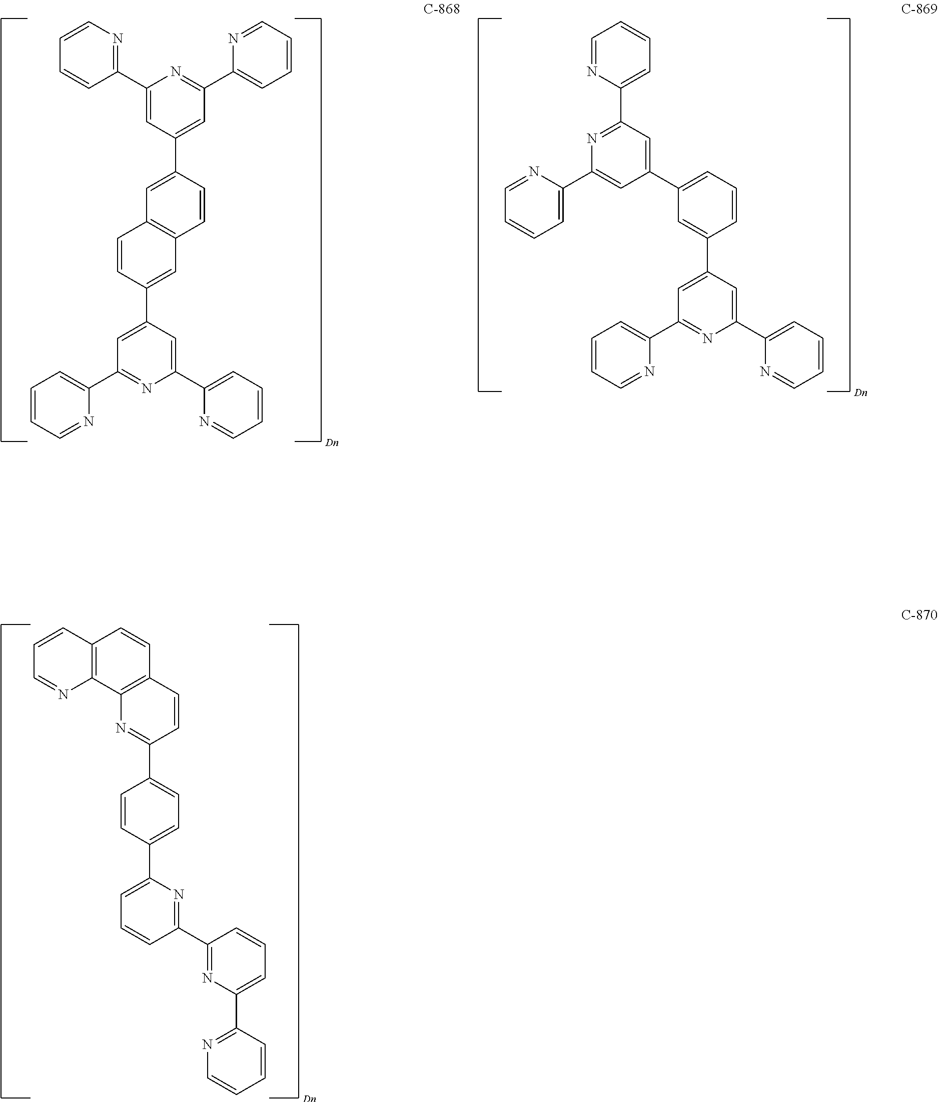

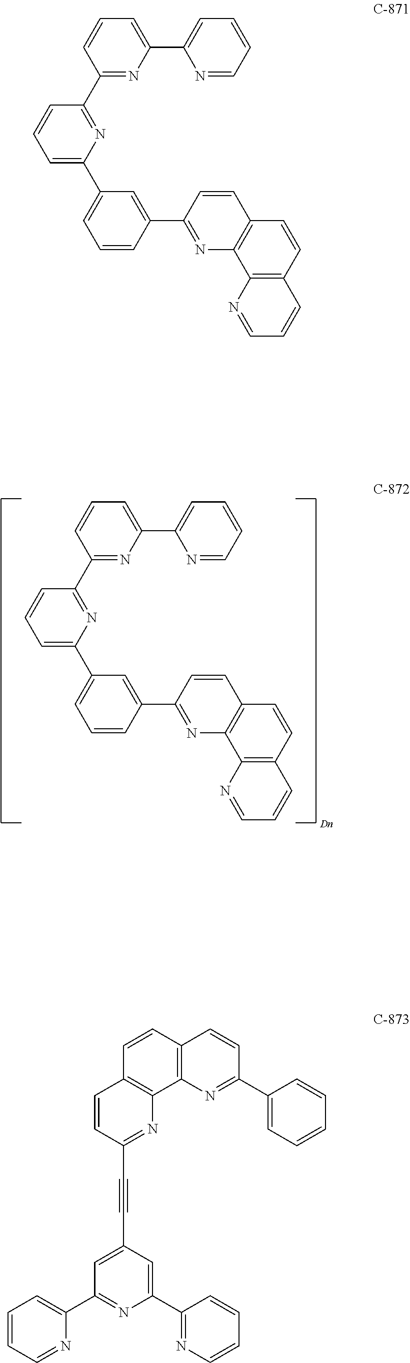

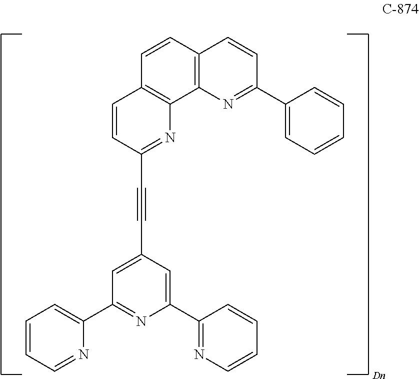

In the compounds above, Dₙ means that n hydrogens are replaced by deuterium, wherein n is an integer of 1 or more, and the maximum value of n is the total number of hydrogens that can be replaced in each compound.

The compound represented by Formula 1 according to the present disclosure may be prepared by synthetic methods known to those skilled in the art. For example, the compound of the present disclosure may be prepared with reference to the following Reaction Schemes 1 to 6, etc., but is not limited thereto.

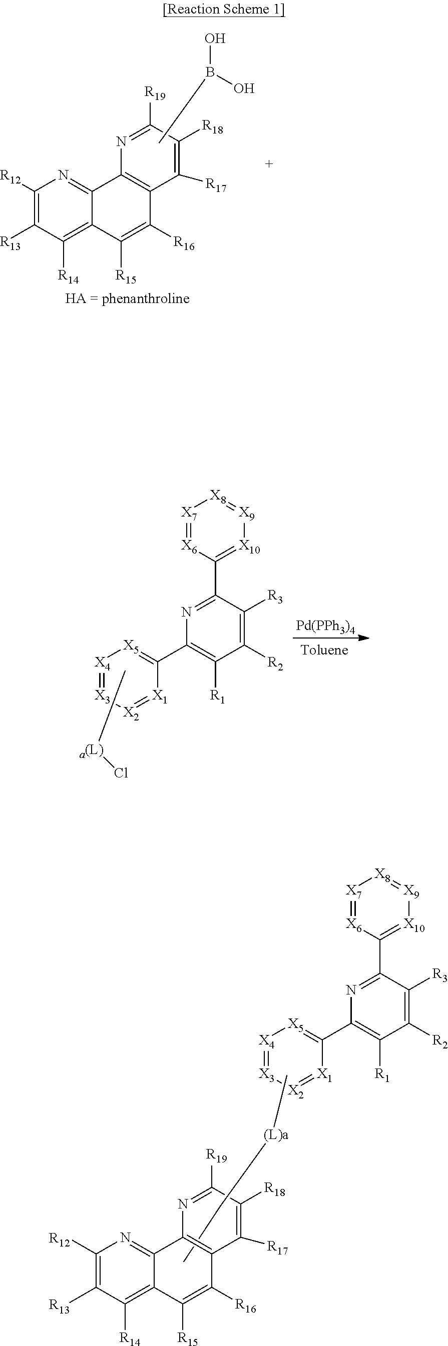

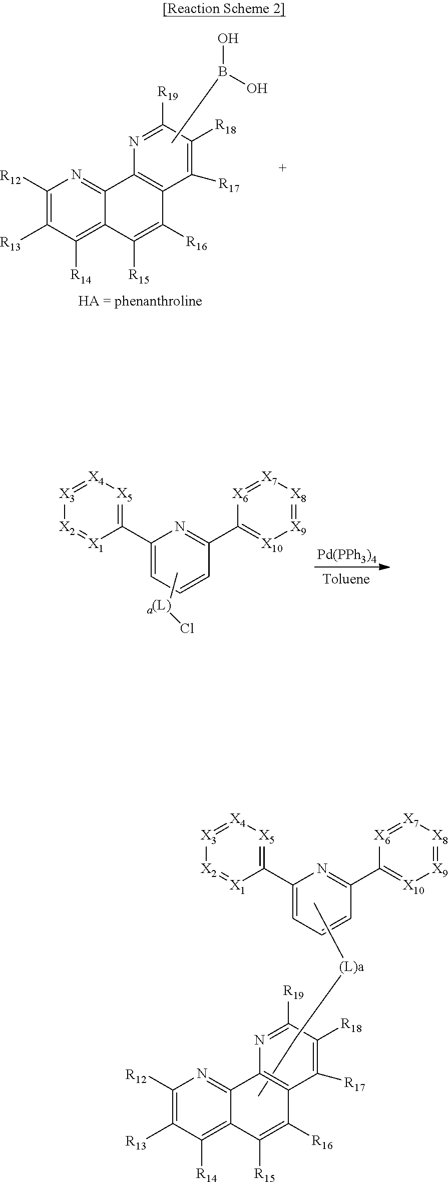

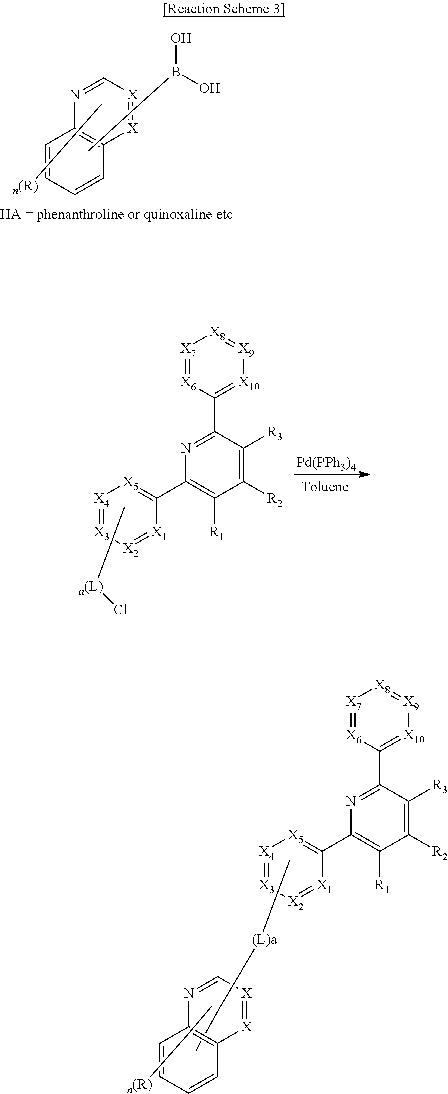

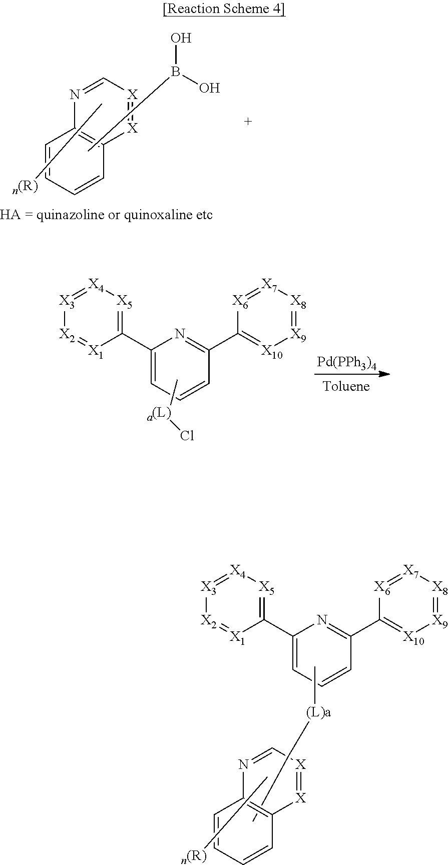

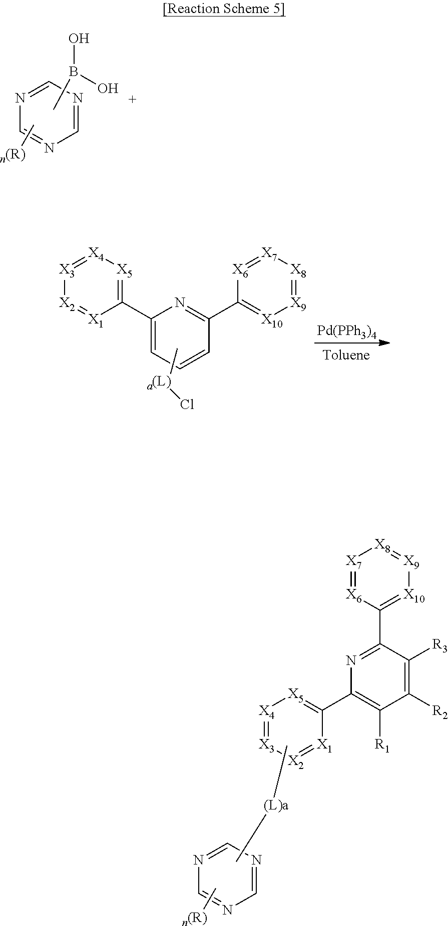

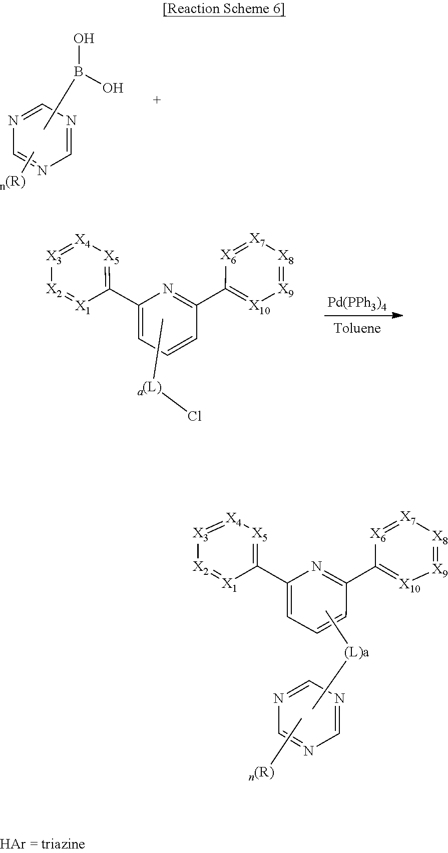

In the Reaction Schemes above, X₁ to X₁₀, R₁ to R₃, L, a, and R₁₂ to R₁₉ are as defined in Formulas 1 and 1-1, X is N or CR, each R is independently as defined for R₁₂, and n is an integer of 1 to 6. When n is an integer of 2 or more, each R may be the same as or different from one another.

The organic electroluminescent compound of the present disclosure may be used in an N-type charge generation layer of an organic electroluminescent device.

Hereinafter, an organic electroluminescent device using the aforementioned organic electroluminescent compound will be described.

An organic electroluminescent device according to one embodiment comprises the organic electroluminescent compound of the present disclosure. According to one embodiment of the present disclosure, the organic electroluminescent compound may be used in an N-type charge generation layer, and may be doped with an additional metal. According to one embodiment of the present disclosure, the metal includes Yb, Li, Cu, Ag, Au, Al, Mg, or any combination thereof.

An organic electroluminescent device according to one embodiment comprises a plurality of light-emitting units positioned between a first electrode and a second electrode; and at least one charge generation layer positioned between adjacent light-emitting units among the plurality of light-emitting units, wherein the charge generation layer comprises the organic electroluminescent compound of the present disclosure. According to one embodiment of the present disclosure, at least one of the plurality of light-emitting units comprises a first light-emitting layer and a second light-emitting layer adjacent to each other.

The organic electroluminescent compound according to one embodiment may be used as a light-emitting material for a white organic light-emitting device. The white organic light-emitting device has been suggested to have various structures such as a side-by-side structure or a stacking structure depending on the arrangement of R (red), G (green) or YG (yellow-green), and B (blue) light-emitting units, or a CCM (color conversion material) method, etc. In addition, the organic electroluminescent compound according to one embodiment may also be used in the organic electroluminescent device comprising a QD (quantum dot).

One of the first electrode and the second electrode may be an anode and the other may be a cathode. In this case, the first electrode and the second electrode may each be formed of a transparent conductive material, or a semi-transparent or reflective conductive material. Depending on the type of materials forming the first electrode and second electrode, the organic electroluminescent device may be a top emission type, a bottom emission type, or a dual-side emission type.

The organic electroluminescent device according to one embodiment of the present disclosure may be an organic electroluminescent device having a tandem structure. In the case of a tandem organic electroluminescent device according to one embodiment, a single light-emitting unit (light-emitting unit) may be formed in a structure in which two or more units are connected by a charge generation layer. The organic electroluminescent device may include a plurality of two or more light-emitting units, for example, a plurality of three or more light-emitting units, having first and second electrodes opposed to each other on a substrate and a light-emitting layer that is stacked between the first and second electrodes and emits light in a specific wavelength range. It may include a plurality of light-emitting units, and each of the light-emitting units may include a hole transport band, a light-emitting layer, and an electron transport band. The hole transport band may include a hole injection layer and a hole transport layer, and the electron transport band may include an electron transport layer and an electron injection layer. According to one embodiment, three or more light-emitting layers may be included in the light-emitting unit. A plurality of light-emitting units may emit the same color or different colors. Additionally, one light-emitting unit may include one or more light-emitting layers, and the plurality of light-emitting layers may be light-emitting layers of the same or different colors. This may include one or more charge generation layers located between each light-emitting unit. The charge generation layer refers to the layer in which holes and electrons are generated when voltage is applied. When there are three or more light-emitting units, a charge generation layer may be located between each light-emitting unit. In this case, the plurality of charge generation layers may be the same as or different from one another. By disposing the charge generation layer between light-emitting units, current efficiency is increased in each light-emitting unit, and charges can be smoothly distributed. Specifically, the charge generation layer is provided between two adjacent stacks and can serve to drive a tandem organic electroluminescent device using only a pair of an anode and a cathode without a separate internal electrode located between the stacks.

The charge generation layer may be composed of an N-type charge generation layer and a P-type charge generation layer, and the N-type charge generation layer may be doped with an alkali metal, an alkaline earth metal, or a compound of an alkali metal and an alkaline earth metal. The alkali metal may include one selected from the group consisting of Li, Na, K, Rb, Cs, Fr, Yb, and combinations thereof, and the alkaline earth metal may include one selected from the group consisting of Be, Mg, Ca, Sr, Ba, Ra, and combinations thereof. The P-type charge generation layer may be made of a metal or an organic material doped with a P-type dopant. For example, the metal may be made of one or two or more alloys selected from the group consisting of Al, Cu, Fe, Pb, Zn, Au, Pt, W, In, Mo, Ni, and Ti. Additionally, commonly used materials may be used as the P-type dopant and host materials used in the P-type doped organic material.

According to one embodiment, the present disclosure can provide a display device comprising an organic electroluminescent compound represented by Formula 1. In addition, it is possible to produce display devices such as smartphones, tablets, notebooks, PCs, TVs, or display devices for vehicles, or lighting devices such as outdoor or indoor lighting by using the organic electroluminescent device of the present disclosure.

Hereinafter, the preparation method and physical properties of the organic electroluminescent compound according to the present disclosure, as well as the driving voltage, current efficiency, and lifespan characteristics of OLEDs according to the present disclosure, will be explained. However, the following examples are provided solely to describe the characteristics of the compounds and OLEDs according to the present disclosure for a more detailed understanding of the present disclosure, and the present disclosure is not limited to the following examples.

### [Example 1] Synthesis of Compound C-42

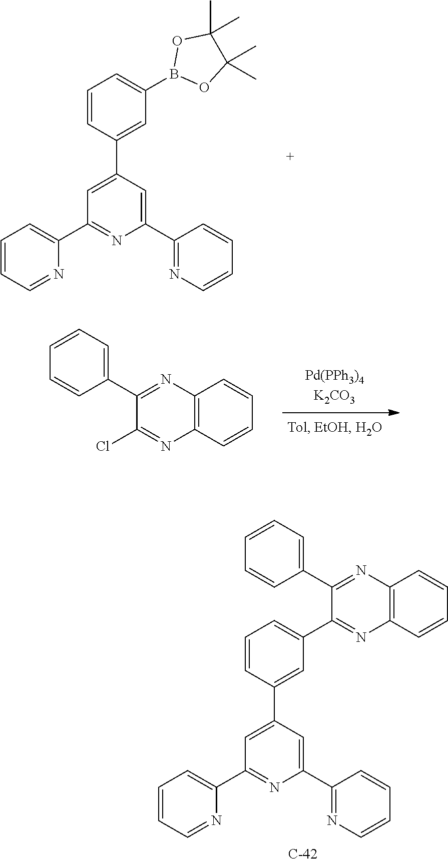

4′-(3-(4,4,5,5-Tetramethyl-1,3,2-dioxaborolan-2-yl)phenyl)-2,2′:6′,2″-terpyridine (10.0 g, 23.0 mmol), 2-chloro-3-phenylquinoxaline (6.08 g, 25.3 mmol), Pd(PPh₃)₄ (1.33 g, 1.15 mmol), and K₂CO₃ (6.35 g, 45.9 mmol) were added to toluene (115 mL), EtOH (23 mL), and distilled water (23 mL) and stirred under reflux at 100° C. After 17 hours, the mixture was cooled to room temperature, extracted with methylene chloride (MC), and then filtered through silica. Thereafter, the residue was recrystallized to obtain Compound C-42 (8.90 g, yield: 75.4%).

| | MW | M.P. |
|---|---|---|
| C-42 | 513.60 | 212° C. |

### [Example 2] Synthesis of Compound C-1

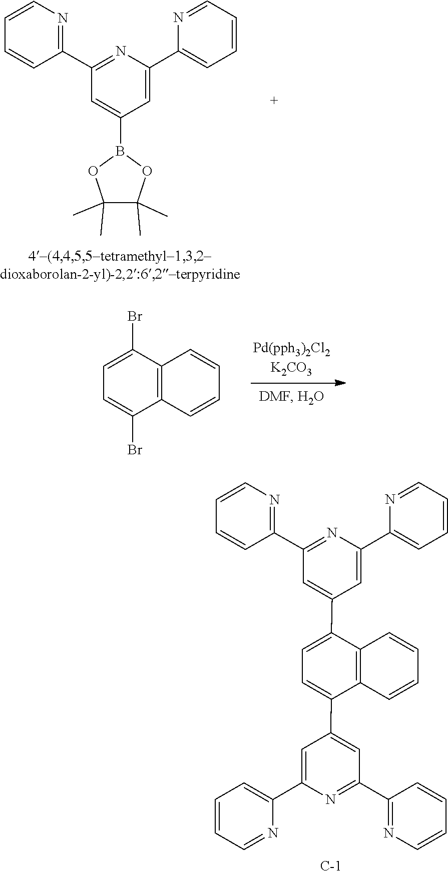

4′-(4,4,5,5-Tetramethyl-1,3,2-dioxaborolan-2-yl)-2,2′:6′,2″-terpyridine (14.8 g, 41.1 mmol), 1,4-dibromonaphthalene (5.6 g, 19.6 mmol), Pd(PPh₃)₂Cl₂ (1.38 g, 1.96 mmol), and K₂CO₃ (13.5 g, 97.9 mmol) were added to dimethylformamide (DMF, 112 mL) and distilled water (28 mL) and stirred under reflux at 110° C. After 16 hours, the mixture was cooled to room temperature, followed by addition of MeOH and stirring, and the resulting precipitated solid was filtered. Thereafter, the residue was filtered through silica and recrystallized to obtain Compound C-1 (7.7 g, yield: 66.6%).

| | MW | M.P. |
|---|---|---|
| C-1 | 590.69 | 355° C. |

### [Example 3] Synthesis of Compound C-873

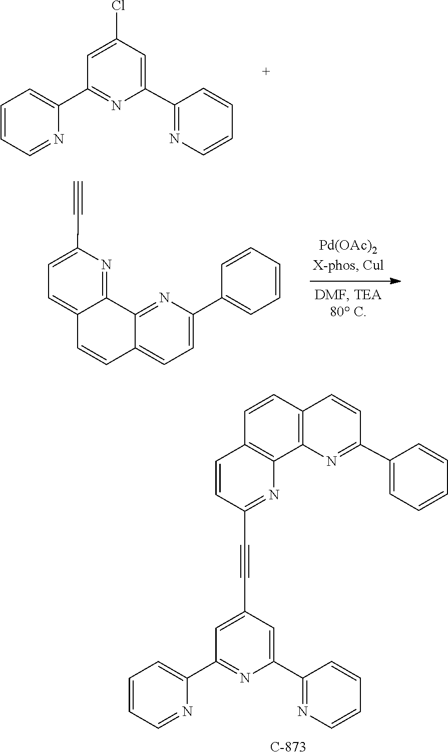

4′-Chloro-2,2′:6′,2″-terpyridine (3.0 g, 11.2 mmol), 2-ethynyl-9-phenyl-1,10-phenanthroline (3.46 g, 12.3 mmol), Pd(OAc)₂ (0.50 g, 2.24 mmol), XPhos (2.1 g, 4.48 mmol), and CuI (0.21 g, 1.12 mmol) were added to DMF (150 mL) and triethylamine (TEA, 30 mL) and stirred at 80° C. After 20 hours, the mixture was cooled to room temperature, filtered through silica, and then recrystallized to obtain Compound C-873 (1.5 g, yield: 26%).

| MW | M.P. |
|---|---|
| C-873 | 511.59 |

### [Example 4] Synthesis of Compound C-437

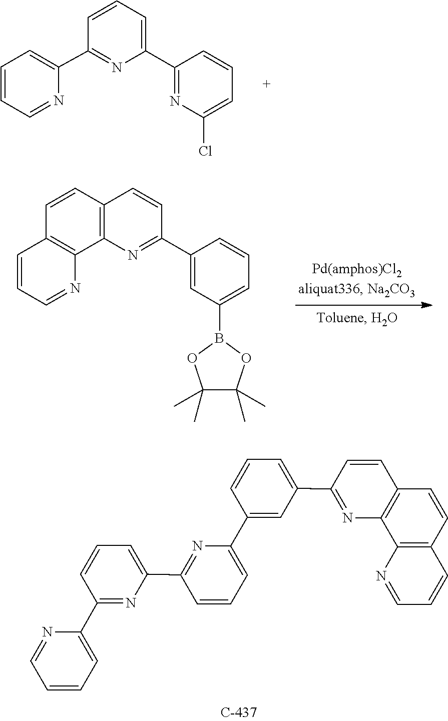

6-Chloro-2,2′:6′,2″-terpyridine (9.0 g, 33.6 mmol), 2-(3-(4,4,5,5-tetramethyl-1,3,2-dioxaborolan-2-yl)phenyl)-1,10-phenanthroline (11 g, 28.0 mmol), Pd(amphos)Cl₂ (1.38 g, 1.96 mmol), Aliquat 336 (1.1 g, 2.80 mmol), and Na₂CO₃ (5.9 g, 56.0 mmol) were added to toluene (140 mL) and distilled water (45 mL), and stirred under reflux at 120° C. After 16 hours, the mixture was cooled to room temperature, extracted with MC, and then filtered through silica. Thereafter, the residue was recrystallized to obtain Compound C-437 (3.3 g, yield: 24%).

| | MW | M.P |
|---|---|---|
| C-437 | 487.57 | 124° C. |

### [Example 5] Synthesis of Compound C-91

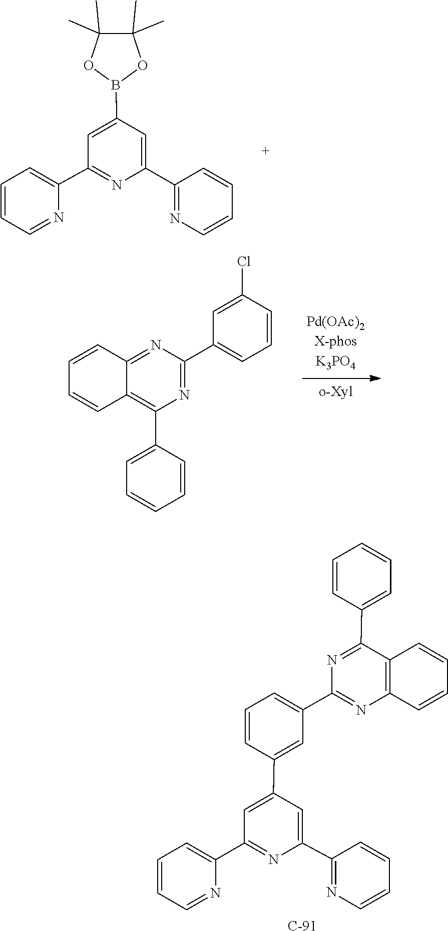

4′-(4,4,5,5-Tetramethyl-1,3,2-dioxaborolan-2-yl)-2,2′:6′,2″-terpyridine (7.5 g, 20.9 mmol), 2-(3-chlorophenyl)-4-phenylquinazoline (6.6 g, 20.9 mmol), Pd(OAc)₂ (0.24 g, 1.04 mmol), XPhos (1.0 g, 2.09 mmol), and K₃PO₄ (13 g, 62.6 mmol) were added to o-xylene (150 mL) and stirred at 120° C. After 6 hours, the mixture was cooled to room temperature, and the resulting solid was filtered. Thereafter, the residue was filtered through silica and recrystallized to obtain Compound C-91 (3.5 g, yield: 33%).

| | MW | M.P. |
|---|---|---|
| C-91 | 513.60 | 228° C. |

### [Example 6] Synthesis of Compound C-120

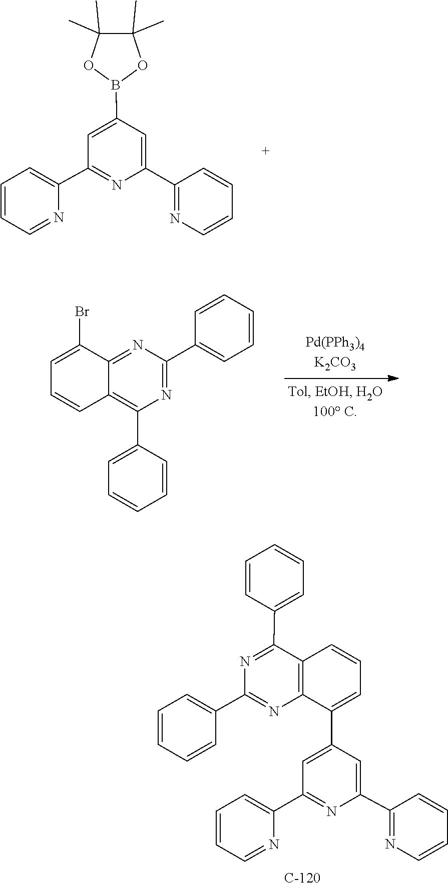

4′-(4,4,5,5-Tetramethyl-1,3,2-dioxaborolan-2-yl)-2,2′:6′,2″-terpyridine (5.5 g, 15.3 mmol), 8-bromo-2,4-diphenylquinazoline (6.1 g, 16.8 mmol), Pd(PPh₃)₄ (2.6 g, 2.30 mmol), and K₂CO₃ (6.3 g, 45.9 mmol) were added to toluene (150 mL), EtOH (30 mL), and distilled water (30 mL) and stirred under reflux at 100° C. After 17 hours, the mixture was cooled to room temperature, and the resulting solid was filtered. Thereafter, the residue was filtered through silica and recrystallized to obtain Compound C-120 (6.4 g, yield: 81%).

| | MW | M.P. |
|---|---|---|
| C-120 | 513.60 | 265° C. |

### [Example 7] Synthesis of Compound C-3

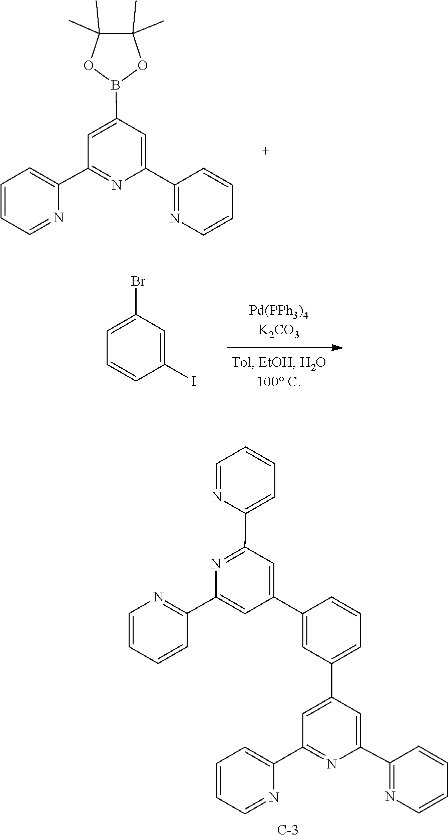

4′-(4,4,5,5-Tetramethyl-1,3,2-dioxaborolan-2-yl)-2,2′:6′,2″-terpyridine (14 g, 38.9 mmol), 3-bromo-iodobenzene (5.0 g, 17.7 mmol), Pd(PPh₃)₄ (1.6 g, 1.41 mmol), and K₂CO₃ (12 g, 88.4 mmol) were added to toluene (200 mL), EtOH (40 mL), and distilled water (40 mL) and stirred under reflux at 100° C. After 16 hours, the mixture was cooled to room temperature, filtered through silica, and then recrystallized to obtain Compound C-3 (6.7 g, yield: 70%).

| | MW | M.P. |
|---|---|---|
| C-3 | 540.63 | 254° C. |

### [Example 8] Synthesis of Compound C-294

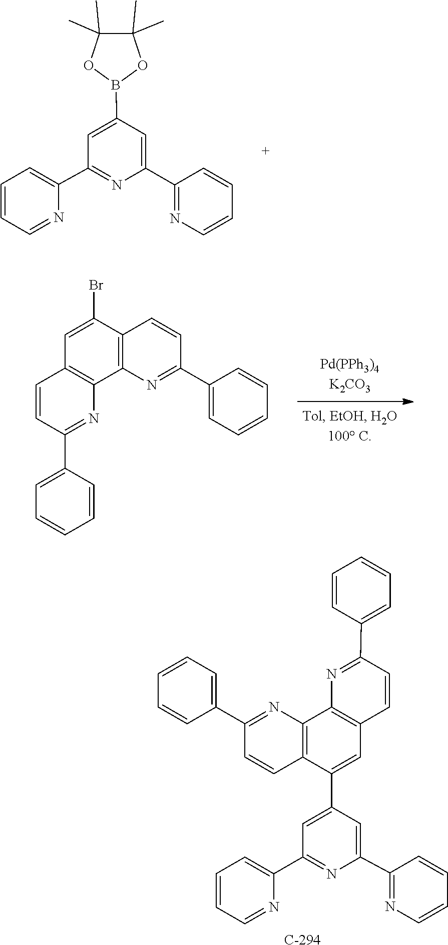

4′-(4,4,5,5-Tetramethyl-1,3,2-dioxaborolan-2-yl)-2,2′:6′,2″-terpyridine (5.5 g, 15.3 mmol), 5-bromo-2,9-diphenyl-1,10-phenanthroline (6.4 g, 15.6 mmol), Pd(PPh₃)₄ (0.89 g, 0.766 mmol), and K₂CO₃ (6.4 g, 45.9 mmol) were added to toluene (150 mL), EtOH (30 mL), and distilled water (30 mL) and stirred under reflux at 100° C. After 24 hours, the mixture was cooled to room temperature, filtered through silica, and then recrystallized to obtain Compound C-294 (6.2 g, yield: 72%).

| | MW | M.P. |
|---|---|---|
| C-294 | 563.66 | 301° C. |

### [Example 9] Synthesis of Compound C-762

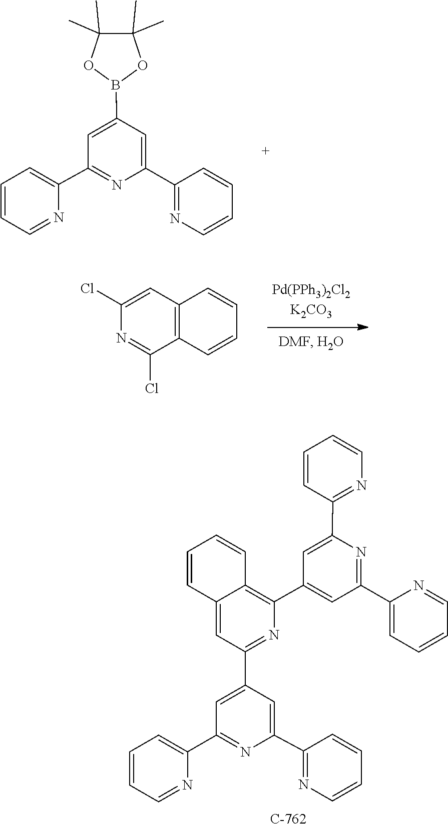

4′-(4,4,5,5-Tetramethyl-1,3,2-dioxaborolan-2-yl)-2,2′:6′,2″-terpyridine (21.8 g, 60.6 mmol), 1,3-dichloroisoquinoline (4.80 g, 24.2 mmol), Pd(PPh₃)₂Cl₂ (1.19 g, 1.70 mmol), and K₂CO₃ (13.4 g, 96.9 mmol) were added to DMF (250 mL) and distilled water (50 mL) and stirred under reflux at 120° C. After 6 hours, the mixture was cooled to room temperature, EtOH (350 mL) was added thereto, and the resulting solid was filtered. Thereafter, the residue was filtered through silica and recrystallized to obtain Compound C-762 (12.3 g, yield: 85.8%).

| | MW | M.P. |
|---|---|---|
| C-762 | 591.68 | 301° C. |

### [Example 10] Synthesis of Compound C-366

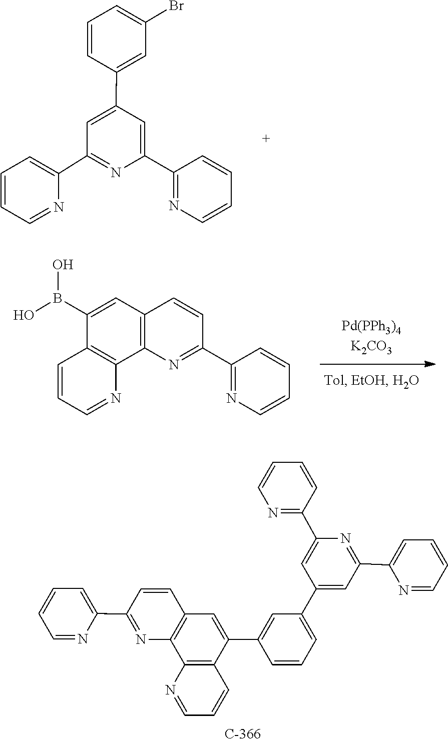 [SMILES: Brc1cccc(-c2cc(-c3ccccn3)nc(-c3ccccn3)c2)c1 ; OB(O)c1cc2ccc(-c3ccccn3)nc2c2ncccc12 ; c1ccc(-c2cc(-c3cccc(-c4cc5ccc(-c6ccccn6)nc5c5ncccc45)c3)cc(-c3ccccn3)n2)nc1]

4′-(3-Bromophenyl)-2,2′:6′,2″-terpyridine (7.00 g, 18.0 mmol), (9-(pyridin-2-yl)-1,10-phenanthrolin-5-yl)boronic acid (6.51 g, 21.6 mmol), Pd(PPh₃)₄ (1.04 g, 0.901 mmol), and K₂CO₃ (7.48 g, 54.1 mmol) were added to toluene (150 mL), EtOH (30 mL), and distilled water (30 mL) and stirred under reflux at 100° C. After 18 hours, the mixture was cooled to room temperature, extracted with dichloromethane (DCM), and then filtered through silica. Thereafter, the residue was crystallized to obtain Compound C-366 (4.5 g, yield: 44%).

| | MW | M.P. |
|---|---|---|
| C-366 | 564.65 | 203° C. |

### [Example 11] Synthesis of Compound C-368

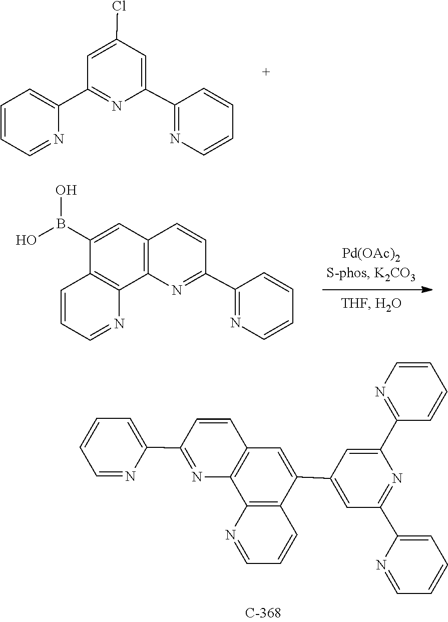

4′-Chloro-2,2′:6′,2″-terpyridine (4.00 g, 15.0 mmol), (9-(pyridin-2-yl)-1,10-phenanthrolin-5-yl)boronic acid (4.95 g, 16.4 mmol), Pd(OAc)₂ (0.168 g, 0.747 mmol), SPhos (0.613 g, 1.49 mmol), and K₂CO₃ (6.20 g, 44.8 mmol) were added to tetrahydrofuran (THF, 150 mL) and distilled water (15 mL) and stirred under reflux at 70° C. After 18 hours, the mixture was cooled to room temperature, and the resulting solid was filtered through silica. Thereafter, the residue was crystallized to obtain Compound C-368 (3.0 g, yield: 41%).

| | MW | M.P. |
|---|---|---|
| C-368 | 488.55 | 304° C. |

### [Example 12] Synthesis of Compound C-665

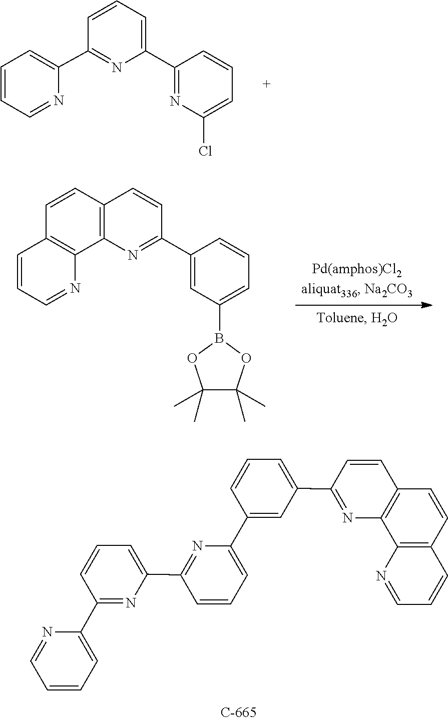

6-Chloro-2,2′:6′,2″-terpyridine (9.00 g, 33.6 mmol), 2-(3-(4,4,5,5-tetramethyl-1,3,2-dioxaborolan-2-yl)phenyl)-1,10-phenanthroline (10.8 g, 28.0 mmol), Pd(amphos)Cl₂ (1.40 g, 1.96 mmol), Aliquat 336 (1.10 g, 2.80 mmol), and Na₂CO₃ (5.90 g, 56.0 mmol) were added to toluene (140 mL) and distilled water (45 mL) and stirred under reflux at 130° C. After 4 hours, the mixture was cooled to room temperature, extracted with DCM, and then filtered through silica. Thereafter, the residue was crystallized to obtain Compound C-665 (3.0 g, yield: 22%).

| | MW | M.P. |
|---|---|---|
| C-665 | 487.57 | 124° C. |

### [Example 13] Synthesis of Compound C-8

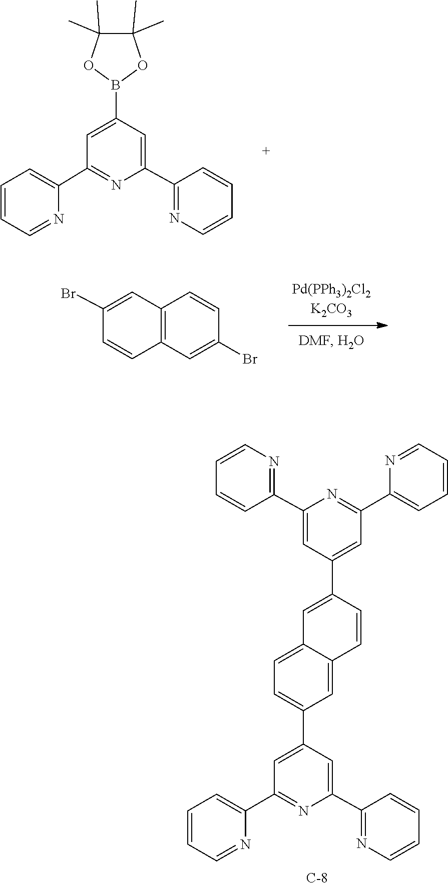

4′-(4,4,5,5-Tetramethyl-1,3,2-dioxaborolan-2-yl)-2,2′:6′,2″-terpyridine (15.5 g, 43.0 mmol), 2,6-dibromonaphthalene (6.0 g, 21.0 mmol), Pd(PPh₃)₂Cl₂ (0.736 g, 1.05 mmol), and K₂CO₃ (8.70 g, 62.9 mmol) were added to DMF (150 mL) and distilled water (30 mL) and stirred under reflux at 120° C. After 18 hours, the mixture was cooled to room temperature, and the resulting solid was filtered. Thereafter, the residue was filtered through silica and crystallized to obtain Compound C-8 (6.6 g, yield: 53%).

| | MW | M.P. |
|---|---|---|
| C-8 | 590.69 | 350° C. |

### [Example 14] Synthesis of Compound C-817

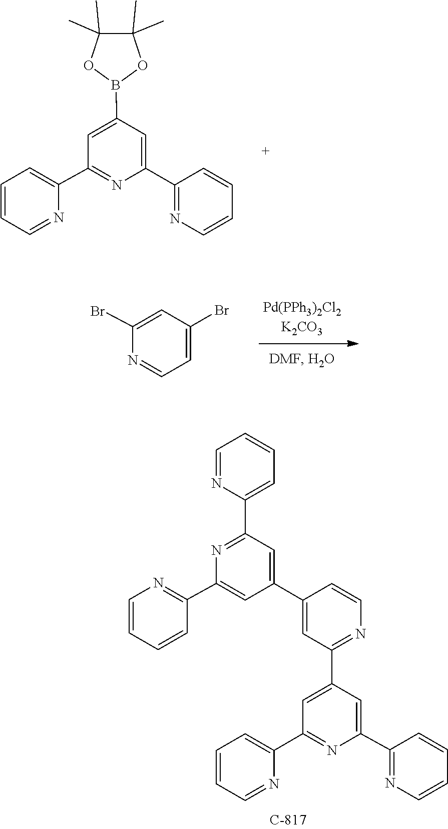 [SMILES: CC1(C)OB(c2cc(-c3ccccn3)nc(-c3ccccn3)c2)OC1(C)C ; Brc1ccnc(Br)c1 ; c1ccc(-c2cc(-c3ccnc(-c4cc(-c5ccccn5)nc(-c5ccccn5)c4)c3)cc(-c3ccccn3)n2)nc1]

4′-(4,4,5,5-Tetramethyl-1,3,2-dioxaborolan-2-yl)-2,2′:6′,2″-terpyridine (15.0 g, 41.9 mmol), 2,4-dibromopyridine (4.4 g, 18.4 mmol), Pd(PPh₃)₂Cl₂ (0.773 g, 1.11 mmol), and K₂CO₃ (12.7 g, 91.8 mmol) were added to DMF (200 mL) and distilled water (40 mL) and stirred under reflux at 120° C. After 18 hours, the mixture was cooled to room temperature, and the resulting solid was filtered. Thereafter, the residue was filtered through silica and crystallized to obtain Compound C-817 (6.4 g, yield: 64%).

| | MW | M.P. |
|---|---|---|
| C-817 | 541.62 | 291° C. |

### [Device Examples 1 to 5] Preparation of Organic Electroluminescent Devices by Depositing the Compounds According to the Present Disclosure as N-Type Charge Generation Layers

OLEDs according to the present disclosure were produced. First, a transparent electrode indium tin oxide (ITO) thin film (10Ω/□) on a glass substrate for an OLED (GEOMATEC CO., LTD.) was subjected to ultrasonic washing with acetone and isopropyl alcohol, sequentially, and was thereafter stored in isopropyl alcohol and then used. The ITO substrate was then mounted on a substrate holder of a vacuum vapor deposition apparatus. Next, Compound HI-1 was introduced into a cell of the vacuum vapor deposition apparatus, and Compound HT-3 was introduced into another cell. The two materials were evaporated at different rates, and Compound HI-1 was deposited in a doping amount of 3 wt % based on the total amount of Compounds HI-1 and HT-3 to form a hole injection layer having a thickness of 5 nm. Compound HT-3 was then deposited on the hole injection layer to form a first hole transport layer having a thickness of 30 nm. Next, Compound HT-4 was introduced into another cell of the vacuum vapor deposition apparatus and was evaporated by applying an electric current to the cell, thereby forming a second hole transport layer having a thickness of 5 nm on the first hole transport layer. After formation of the hole injection layer and the hole transport layers, a first light-emitting layer was deposited thereon as follows: Compound H-1 was introduced into a cell of the vacuum vapor deposition apparatus as a host, and Compound D-1 was introduced into another cell as a dopant. The two materials were evaporated at different rates, and the dopant was deposited in a doping amount of 2 wt % based on the total amount of the host and the dopant to form a first light-emitting layer having a thickness of 20 nm on the second hole transport layer. Next, Compound ET-1 was deposited as a first hole-blocking layer material to a thickness of 5 nm on the first light-emitting layer. Thereafter, Compound ET-2 was deposited as an electron transport layer material to a thickness of 10 nm to form a first electron transport layer on the first hole-blocking layer. Subsequently, lithium (Li) was deposited in an amount of 0.5 wt % on the compounds shown in Tables 1 and 2 below to form an N-type charge generation layer having a thickness of 4 nm on the first electron transport layer. Next, Compound HI-1 was deposited in a doping amount of 6 wt % based on the total amount of Compounds HI-1 and HT-3 to form a P-type charge generation layer having a thickness of 10 nm on the N-type charge generation layer. After deposition of Compound HT-3 to a thickness of 30 nm to form a third hole transport layer, Compound HT-4 was deposited to a thickness of 5 nm to form a fourth hole transport layer. Next, a second light-emitting layer was deposited thereon as follows: Compound H-1 was introduced into a cell of the vacuum vapor deposition apparatus as a host, and Compound D-1 was introduced into another cell as a dopant. The two materials were evaporated at different rates, and the dopant was deposited in a doping amount of 2 wt % based on the total amount of the host and the dopant to form a second light-emitting layer having a thickness of 20 nm on the fourth hole transport layer. Compound ET-1 was deposited as a second hole-blocking layer material to a thickness of 5 nm on the second light-emitting layer. Thereafter, Compound ET-2 and Compound EI-1 were respectively introduced into two cells of the vacuum vapor deposition apparatus as second electron transport layer materials, and the two materials were deposited at a weight ratio of 2:1 to form a second electron transport layer having a thickness of 25 nm. After deposition of ytterbium (Yb) as an electron injection layer to a thickness of 1 nm on the second electron transport layer, an Al cathode having a thickness of 80 nm was deposited on the electron injection layer by another vacuum vapor deposition apparatus. Thus, OLEDs were produced. Each compound used for all of the materials was purified by vacuum sublimation at 10⁻⁶ Torr.

### [Comparative Examples 1 and 2] Preparation of Organic Electroluminescent Devices by Depositing a Conventional Compound as an N-Type Charge Generation Layer

OLEDs were produced in the same manner as in Device Example 1, except that the compound shown in Tables 1 and 2 below was used in the N-type charge generation layer.

The driving voltage, current efficiency, the time taken for luminance to decrease from 100% to 95% (lifespan; T₉₅) under 2×acceleration, and progressive driving voltage changes (ΔV) for 10 hours under 2×acceleration conditions at a luminance of 1,000 nit of the organic electroluminescent devices produced in Device Examples 1 to 5 and Comparative Examples 1 and 2 were measured, and the results thereof are provided in Tables 1 and 2 below.

TABLE 1

| | N-Type Charge | Driving | Current | Lifespan |
|---|---|---|---|---|
| | Generation | Voltage | Efficiency | T₉₅ |
| | Layer | (V) | [cd/A] | [hr] |
| Device | C-42 | 6.4 | 7.9 | 193.2 |
| Example 1 | | | | |
| Device | C-406 | 6.2 | 7.5 | 169.0 |
| Example 2 | | | | |
| Comparative | T-1 | 6.4 | 7.5 | 167.0 |
| Example 1 | | | | |

TABLE 2

| | | | | Progressive |
|---|---|---|---|---|
| | N-Type Charge | Driving | Current | Driving Voltage |
| | Generation | Voltage | Efficiency | ΔV |
| | Layer | [V] | [cd/A] | [%] |
| Device | C-1 | 6.4 | 7.6 | 102.3 |
| Example 3 | | | | |
| Device | C-366 | 6.4 | 7.5 | 101.8 |
| Example 4 | | | | |
| Device | C-8 | 6.4 | 7.5 | 100.8 |
| Example 5 | | | | |
| Comparative | T-1 | 6.4 | 7.5 | 102.7 |
| Example 2 | | | | |

From Tables 1 and 2 above, it can be confirmed that the organic electroluminescent devices in which the organic electroluminescent compounds according to the present disclosure are used in the N-type charge generation layers exhibit lower progressive driving voltage and/or higher current efficiency and/or longer lifespan characteristics compared to a case where the conventional compound is used.

### [Device Example 6] Preparation of Devices for Measuring Lateral Resistivity by Depositing the Compound According to the Present Disclosure as an N-Type Charge Generation Layer

Devices according to the present disclosure were produced. First, a transparent electrode indium tin oxide (ITO) thin film on a glass substrate for an OLED (GEOMATEC CO., LTD.) was subjected to ultrasonic washing with acetone and isopropyl alcohol, sequentially, and was thereafter stored in isopropyl alcohol and then used. The ITO substrate was then mounted on a substrate holder of a vacuum vapor deposition apparatus. Next, Li was respectively deposited in an amount of 0.5 wt %, 1 wt %, or 2 wt % on the compound shown in Table 3 below to form an N-type charge generation layer having a thickness of 100 nm. Thus, devices for measuring lateral resistivity were produced.

### [Comparative Example 3] Preparation of Devices for Measuring Lateral Resistivity by Depositing a Conventional Compound as an N-Type Charge Generation Layer

Devices for measuring lateral resistivity were produced in the same manner as in Device Example 6, except that the compound shown in Table 3 below was used in the N-type charge generation layer.

The lateral resistivity of the devices produced in Device Example 6 and Comparative Example 3 were measured, and the results thereof are provided in Table 3 below.

The lateral resistivity was measured using a separately manufactured four-terminal resistance meter on a device in which two unconnected electrodes were connected by co-deposition of an N-charge generating material and Li. Current was supplied to one electrode, and the resistance generated by the current flowing from the opposite electrode was measured, thereby securing a high-precision resistivity.

TABLE 3

| | N-Type Charge | Lateral Resistivity (kΩ) | | |
|---|---|---|---|---|
| | Generation Layer | Li 0.5% | Li 1% | Li 2% |
| Device | C-42 | ∞ | 67,500 | 6,350 |
| Example 6 | | | | |
| Comparative | T-1 | 861 | 341 | 33 |
| Example 3 | | | | |

The lateral resistivity is a numerical value representing the resistance to leakage current in the N-type charge generation layer. As the lateral resistivity increases, the lateral leakage current decreases. From Table 3 above, it can be confirmed that the organic electroluminescent devices in which the organic electroluminescent compound according to the present disclosure is used in the N-type charge generation layer exhibit higher lateral resistivity compared to a case where the conventional compound is used. Consequently, it can be seen to improve the efficiency of the device, such as current efficiency, lifespan, etc., by relative limitation of the current that flows unnecessarily laterally in the device.

### [Device Examples 7 and 8] Preparation of Organic Electroluminescent Devices by Depositing the Compounds According to the Present Disclosure as N-Type Charge Generation Layers

OLEDs were produced in the same manner as in Device Example 1, except that first electron transport layers were deposited to a thickness of 12 nm, ytterbium (Yb) was deposited in an amount of 2 wt % on the compounds shown in Table 4 below to form N-type charge generation layers having a thickness of 9 nm on the first electron transport layers, and P-type charge generation layers were deposited to a thickness of 6 nm on the N-type charge generation layers.

### [Comparative Example 4] Preparation of an Organic Electroluminescent Device by Depositing a Conventional Compound as an N-Type Charge Generation Layer

An OLED was produced in the same manner as in Device Example 7, except that the compound shown in Table 4 below was used in an N-type charge generation layer.

The respective progressive driving voltage changes (ΔV) for 10 hours under 2×acceleration conditions of the organic electroluminescent devices produced in Device Examples 7 and 8 and Comparative Example 4 were measured, and the results thereof are provided in Table 4 below.

TABLE 4

| | | Progressive |
|---|---|---|
| | | Driving Voltage |
| | N-Type Charge | ΔV |
| | Generation Layer | [%] |
| Device Example 7 | C-324 | 102.5 |
| Device Example 8 | C-3 | 102.5 |
| Comparative Example 4 | T-1 | 103.6 |

From Table 4 above, it can be confirmed that the organic electroluminescent devices in which the organic electroluminescent compounds according to the present disclosure are used in the N-type charge generation layers exhibit lower progressive driving voltage characteristics compared to a case where the conventional compound is used. Consequently, it can be expected that the organic electroluminescent devices according to the present disclosure will exhibit good voltage stability, reduced power consumption, and long lifespan characteristics.

### [Device Example 9] Preparation of an Organic Electroluminescent Device by Depositing the Compound According to the Present Disclosure as an N-Type Charge Generation Layer

An OLED was produced in the same manner as in Device Example 7, except that Compound ET-3 was used as a first electron transport layer material, Compound H-2-D17 was used as a host of a light-emitting layer, and ytterbium (Yb) was deposited on the compound shown in Table 5 below to form an N-type charge generation layer.

### [Comparative Example 5] Preparation of an Organic Electroluminescent Device by Depositing a Conventional Compound as an N-Type Charge Generation Layer

An OLED was produced in the same manner as in Device Example 9, except that the compound shown in Table 5 below was used in the N-type charge generation layer.

The respective progressive driving voltage changes (ΔV) for 10 hours under 2×acceleration conditions of the organic electroluminescent devices produced in Device Example 9 and Comparative Example 5 were measured, and the results thereof are provided in Table 5 below.

TABLE 5

| | | Progressive |
|---|---|---|
| | | Driving Voltage |
| | N-Type Charge | ΔV |
| | Generation Layer | [%] |
| Device Example 9 | C-762 | 102.3 |
| Comparative Example 5 | T-2 | 105.1 |

From Table 5 above, it can be confirmed that the organic electroluminescent device in which the organic electroluminescent compound according to the present disclosure is used in the N-type charge generation layer exhibits lower progressive driving voltage characteristics compared to a case where the conventional compound is used.

### [Device Example 10] Preparation of Electron-Driving Devices by Depositing the Compound According to the Present Disclosure as an N-Type Charge Generation Layer

An electron-driving device according to the present disclosure was produced. First, a transparent electrode indium tin oxide (ITO) thin film (10Ω/□) on a glass substrate for an OLED (GEOMATEC CO., LTD.) was subjected to ultrasonic washing with acetone and isopropyl alcohol, sequentially, and was thereafter stored in isopropyl alcohol and then used. The ITO substrate was then mounted on a substrate holder of a vacuum vapor deposition apparatus. Next, Compound EI-1 was introduced into a cell of the vacuum vapor deposition apparatus and evaporated to deposit a hole-blocking layer. Thereafter, the compound shown in Table 6 below was deposited alone to form an N-type charge generation layer having a thickness of 30 nm on the hole-blocking layer. After deposition of ytterbium (Yb) as an electron injection layer to a thickness of 2 nm on the N-type charge generation layer, an Al cathode having a thickness of 80 nm was deposited on the electron injection layer by another vacuum vapor deposition apparatus. Thus, an electron-driving device was produced. Each compound used for all of the materials was purified by vacuum sublimation at 10⁻⁶ Torr.

Separately, another electron-driving device was produced in the same manner as described above, except that ytterbium (Yb) was deposited in an amount of 2 wt % on the compound shown in Table 6 below to form an N-type charge generation layer.

### [Comparative Example 6] Preparation of Electron-Driving Devices by Depositing a Conventional Compound as an N-Type Charge Generation Layer

Electron-driving devices were respectively produced in the same manner as in Device Example 10, except that the compound shown in Table 6 below was used in the N-type charge generation layer.

The respective driving voltages at 100 mA/cm² of the electron-driving devices comprising a metal-undoped N-type charge generation layer and the electron-driving devices comprising an ytterbium-doped N-type charge generation layer produced in Device Example 10 and Comparative Example 6, and the difference in driving voltages (ΔV), were measured and the results thereof are provided in Table 6 below.

TABLE 6

| | | Driving | Driving | |
|---|---|---|---|---|
| | | Voltage | Voltage | |
| | | (Metal- | (Ytterbium- | |
| | | Undoped N- | Doped N- | |
| | N-Type | Type Charge | Type Charge | Δ |
| | Charge | Generation | Generation | Driving |
| | Generation | Layer) | Layer) | Voltage |
| | Layer | [V] | [V] | [V] |
| Device | C-871 | 3.7 | 2.4 | 1.3 |
| Example 10 | | | | |
| Comparative | T-2 | 3.8 | 3.1 | 0.7 |
| Example 6 | | | | |

From Table 6 above, it can be confirmed that the electron-driving devices in which the organic electroluminescent compound according to the present disclosure used in the N-type charge generation layer exhibit a large difference in driving voltages depending on metal doping compared to a case where the conventional compound is used. In addition, it can be confirmed that as the driving voltage difference increases, the organic molecule binds more firmly to the metal.

The compounds used in Device Examples 1 to 10 and Comparative Examples 1 to 6 above are shown in the following Table 7.

TABLE 7

| | |
|---|---|
| Hole Injection Layer/Hole Transport Layer/P- Type Charge Generation Layer | 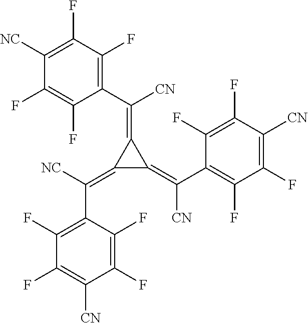 |
| | HI-1 |
| | 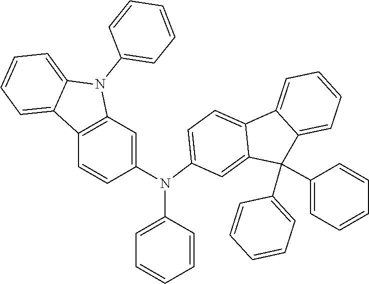 [SMILES: c1ccc(N(c2ccc3c(c2)C(c2ccccc2)(c2ccccc2)c2ccccc2-3)c2ccc3c4ccccc4n(-c4ccccc4)c3c2)cc1] |
| | HT-3 |
| | 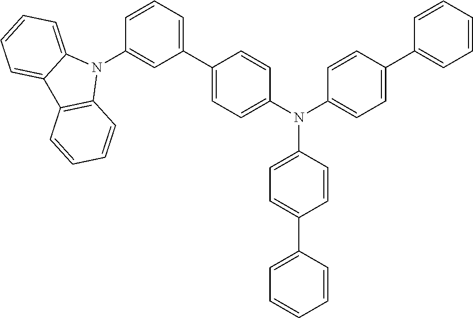 [SMILES: c1ccc(-c2ccc(N(c3ccc(-c4ccccc4)cc3)c3ccc(-c4cccc(-n5c6ccccc6c6ccccc65)c4)cc3)cc2)cc1] |
| | HT-4 |
| Light- Emitting Layer | 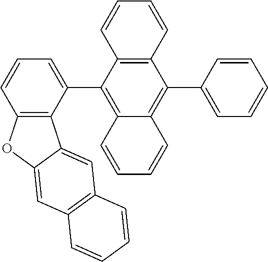 [SMILES: c1ccc(-c2c3ccccc3c(-c3cccc4oc5cc6ccccc6cc5c34)c3ccccc23)cc1] |
| | H-1 |
| | 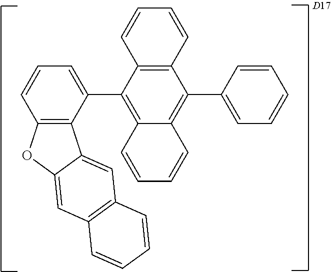 |
| | H-2-D17 |
| | 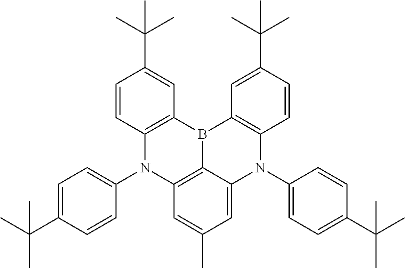 [SMILES: Cc1cc2c3c(c1)N(c1ccc(C(C)(C)C)cc1)c1ccc(C(C)(C)C)cc1B3c1cc(C(C)(C)C)ccc1N2c1ccc(C(C)(C)C)cc1] |
| | D-1 |
| Electron Transport Layer/Hole- Blocking Layer/ | 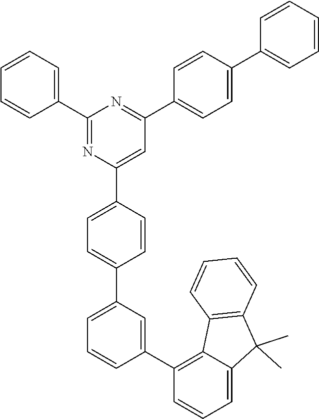 [SMILES: CC1(C)c2ccccc2-c2c(-c3cccc(-c4ccc(-c5cc(-c6ccc(-c7ccccc7)cc6)nc(-c6ccccc6)n5)cc4)c3)cccc21] |
| | ET-1 |
| | 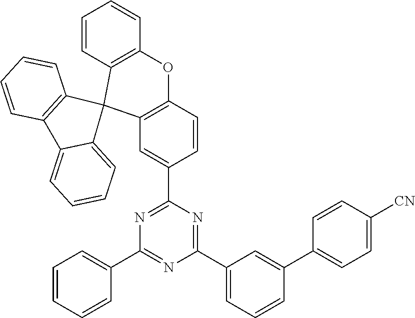 [SMILES: N#Cc1ccc(-c2cccc(-c3nc(-c4ccccc4)nc(-c4ccc5c(c4)C4(c6ccccc6O5)c5ccccc5-c5ccccc54)n3)c2)cc1] |
| | ET-2 |
| | 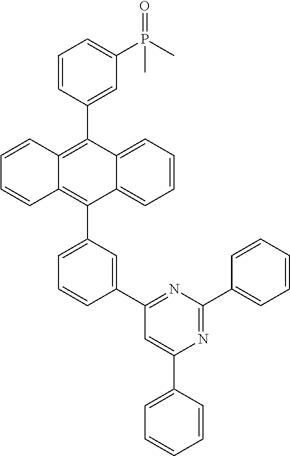 [SMILES: CP(C)(=O)c1cccc(-c2c3ccccc3c(-c3cccc(-c4cc(-c5ccccc5)nc(-c5ccccc5)n4)c3)c3ccccc23)c1] |
| | ET-3 |
| | 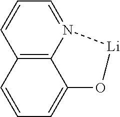 |
| | EI-1 |
| N-Type Charge Generation Layer | 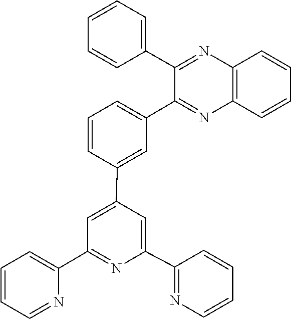 [SMILES: c1ccc(-c2nc3ccccc3nc2-c2cccc(-c3cc(-c4ccccn4)nc(-c4ccccn4)c3)c2)cc1] |
| | C-42 |
| | 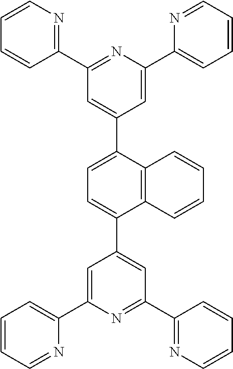 [SMILES: c1ccc(-c2cc(-c3ccc(-c4cc(-c5ccccn5)nc(-c5ccccn5)c4)c4ccccc34)cc(-c3ccccn3)n2)nc1] |
| | C-1 |
| | 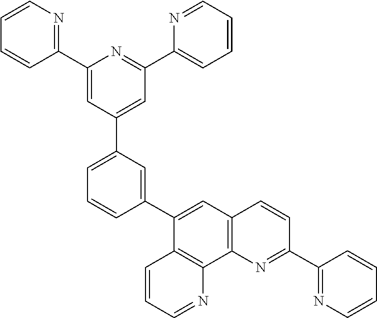 [SMILES: c1ccc(-c2cc(-c3cccc(-c4cc5ccc(-c6ccccn6)nc5c5ncccc45)c3)cc(-c3ccccn3)n2)nc1] |
| | C-366 |
| | 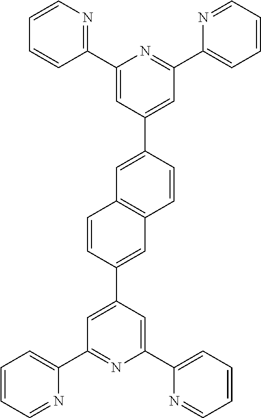 [SMILES: c1ccc(-c2cc(-c3ccc4cc(-c5cc(-c6ccccn6)nc(-c6ccccn6)c5)ccc4c3)cc(-c3ccccn3)n2)nc1] |
| | C-8 |
| | 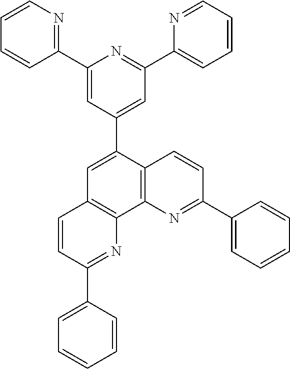 [SMILES: c1ccc(-c2ccc3cc(-c4cc(-c5ccccn5)nc(-c5ccccn5)c4)c4ccc(-c5ccccc5)nc4c3n2)cc1] |
| | C-324 |
| | 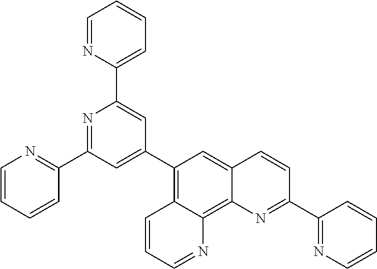 [SMILES: c1ccc(-c2cc(-c3cc4ccc(-c5ccccn5)nc4c4ncccc34)cc(-c3ccccn3)n2)nc1] |
| | C-406 |
| | 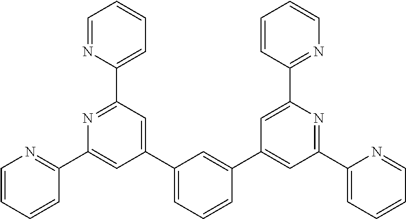 [SMILES: c1ccc(-c2cc(-c3cccc(-c4cc(-c5ccccn5)nc(-c5ccccn5)c4)c3)cc(-c3ccccn3)n2)nc1] |
| | C-3 |
| | 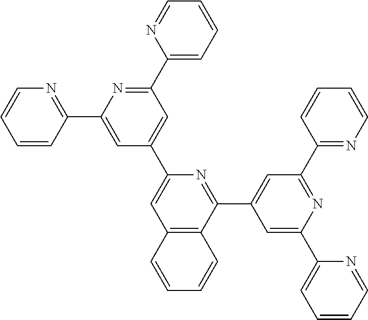 [SMILES: c1ccc(-c2cc(-c3cc4ccccc4c(-c4cc(-c5ccccn5)nc(-c5ccccn5)c4)n3)cc(-c3ccccn3)n2)nc1] |
| | C-762 |
| | 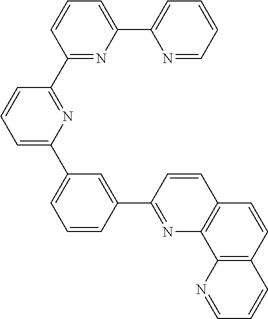 [SMILES: c1ccc(-c2cccc(-c3cccc(-c4cccc(-c5ccc6ccc7cccnc7c6n5)c4)n3)n2)nc1] |
| | C-871 |
| | 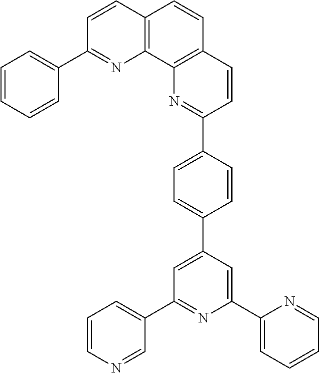 [SMILES: c1ccc(-c2ccc3ccc4ccc(-c5ccc(-c6cc(-c7cccnc7)nc(-c7ccccn7)c6)cc5)nc4c3n2)cc1] |
| | T-1 |
| | 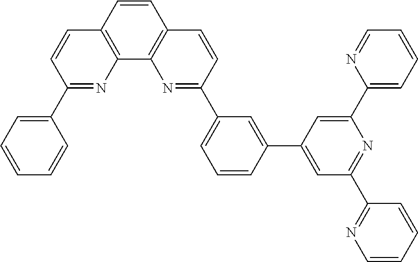 [SMILES: c1ccc(-c2ccc3ccc4ccc(-c5cccc(-c6cc(-c7ccccn7)nc(-c7ccccn7)c6)c5)nc4c3n2)cc1] |
| | T-2 |

1. An organic electroluminescent compound represented by the following Formula 1: 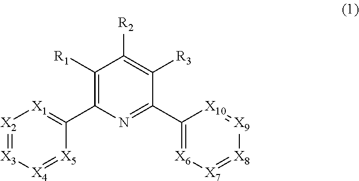
wherein,
X₁ to X₁₀ each independently represent CR₄ or N;
R₁ to R₄ each independently represent hydrogen, deuterium, a halogen, a cyano, a substituted or unsubstituted (C₁-C₃₀)alkyl, a substituted or unsubstituted (C₂-C₃₀)alkenyl, a substituted or unsubstituted (C₃-C₃₀)cycloalkyl, a substituted or unsubstituted (C₃-C₃₀)cycloalkenyl, a substituted or unsubstituted (3- to 7-membered)heterocycloalkyl, or -(L)ₐ-HAr;
at least one of R₁ to R₄ is -(L)ₐ-HAr;
when R₂ is -(L)ₐ-HAr, each R₄ independently represents hydrogen, deuterium, or a cyano group;
each L independently represents a single bond, a substituted or unsubstituted (C₆-C₃₀)arylene, a substituted or unsubstituted (C₂-C₃₀)alkenylene, a substituted or unsubstituted (C₂-C₃₀)alkynylene, or a substituted or unsubstituted (3- to 30-membered)heteroarylene;
a is an integer of 1 or 2, and when a is 2, L may be the same as or different from the other; and
HAr represents a substituted or unsubstituted (3- to 30-membered)heteroaryl comprising at least one nitrogen atom;
provided that where R₂ is -(L)ₐ-HAr and HAr is a substituted or unsubstituted phenanthrolinyl, L is linked to any one of positions 3 to 8 of the phenanthrolinyl when L is not a substituted or unsubstituted (C₂-C₃₀)alkenylene or a substituted or unsubstituted (C₂-C₃₀)alkynylene.

2. The organic electroluminescent compound according to claim 1, wherein the substituted alkyl, the substituted alkenyl(ene), the substituted alkynyl(ene), the substituted aryl(ene), the substituted heteroaryl(ene), the substituted cycloalkyl, the substituted cycloalkenyl, and the substituted heterocycloalkyl are each independently substituted with at least one selected from the group consisting of deuterium, a halogen, a cyano, carboxyl, nitro, hydroxyl, (C₁-C₃₀)alkyl, halo(C₁-C₃₀)alkyl, (C₂-C₃₀)alkenyl, (C₂-C₃₀)alkynyl, (C₁-C₃₀)alkoxy, (C₁-C₃₀)alkylthio, (C₃-C₃₀)cycloalkyl, (C₃-C₃₀)cycloalkenyl, (3- to 7-membered)heterocycloalkyl, (C₆-C₃₀)aryloxy, (C₆-C₃₀)arylthio, (3- to 30-membered)heteroaryl, (C₆-C₃₀)aryl, tri(C₁-C₃₀)alkylsilyl, tri(C₆-C₃₀)arylsilyl, di(C₁-C₃₀)alkyl(C₆-C₃₀)arylsilyl, (C₁-C₃₀)alkyldi(C₆-C₃₀)arylsilyl, a fused ring group of a (C₃-C₃₀)aliphatic ring(s) and a (C₆-C₃₀)aromatic ring(s), amino, mono- or di(C₁-C₃₀)alkylamino, mono- or di(C₆-C₃₀)arylamino, (C₁-C₃₀)alkyl(C₆-C₃₀)arylamino, mono- or di(3- to 30-membered)heteroarylamino, (C₁-C₃₀)alkyl(3- to 30-membered)heteroarylamino, (C₆-C₃₀)aryl(3- to 30-membered)heteroarylamino, (C₁-C₃₀)alkylcarbonyl, (C₁-C₃₀)alkoxycarbonyl, (C₆-C₃₀)arylcarbonyl, (C₆-C₃₀)arylphosphinyl, di(C₆-C₃₀)arylboronyl, di(C₁-C₃₀)alkylboronyl, (C₁-C₃₀)alkyl(C₆-C₃₀)arylboronyl, (C₆-C₃₀)ar(C₁-C₃₀)alkyl, (C₁-C₃₀)alkyl(C₆-C₃₀)aryl, and a combination thereof.

3. The organic electroluminescent compound according to claim 1, wherein the HAr is represented by any one of the following Formulas 1-1 to 1-5: 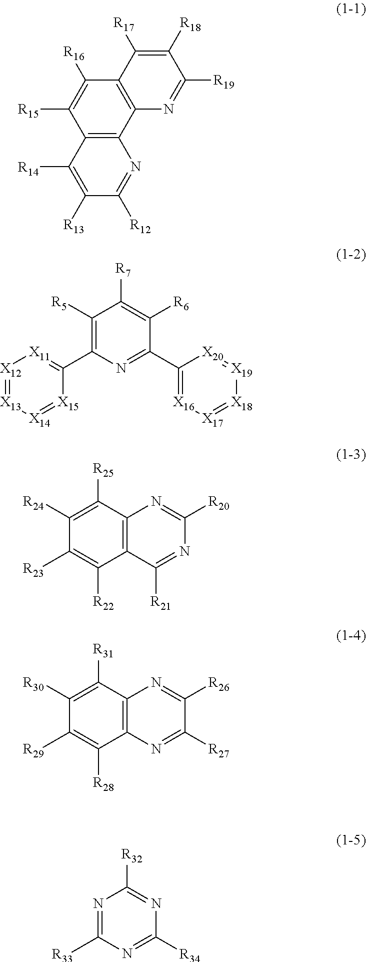
wherein,
X₁₁ to X₂₀ each independently represent CR₈ or N;
R₁₂ and R₁₉ each independently represent hydrogen, deuterium, a halogen, a cyano, a substituted or unsubstituted (C₁-C₃₀)alkyl, a substituted or unsubstituted (C₃-C₃₀)cycloalkyl, a substituted or unsubstituted (C₆-C₃₀)aryl, or a substituted or unsubstituted (3- to 30-membered)heteroaryl;
R₅ to R₈, R₁₃ to R₁₈, and R₂₀ to R₃₄ each independently represent hydrogen, deuterium, a halogen, a cyano, a substituted or unsubstituted (C₁-C₃₀)alkyl, a substituted or unsubstituted (C₃-C₃₀)cycloalkyl, a substituted or unsubstituted (C₆-C₃₀)aryl, a substituted or unsubstituted (3- to 30-membered)heteroaryl, or L;
any one of R₅ to R₈ is L, any one of R₁₃ to R₁₈ is L, any one of R₂₀ to R₂₅ is L, any one of R₂₆ to R₃₁ is L, and any one of R₃₂ to R₃₄ is L; and
L is as defined in claim 1.

4. The organic electroluminescent compound according to claim 1, wherein the -(L)ₐ-HAr is represented by any one of the following Formulas 1-6 to 1-25: 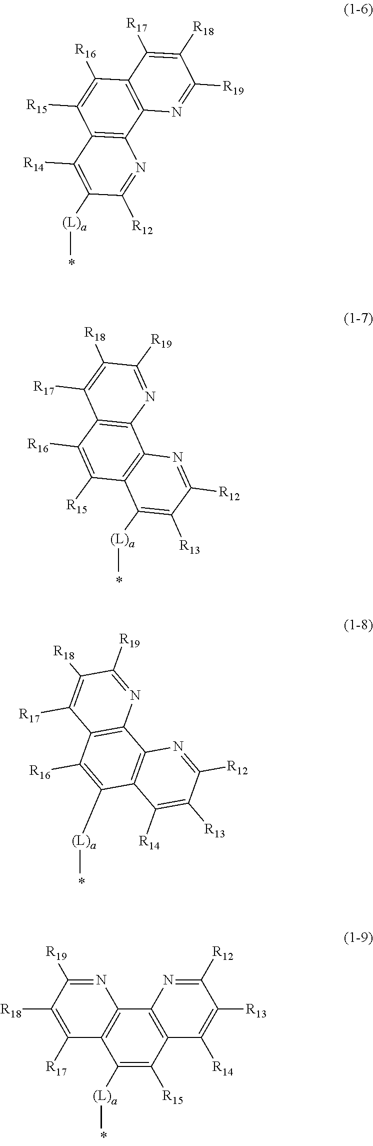 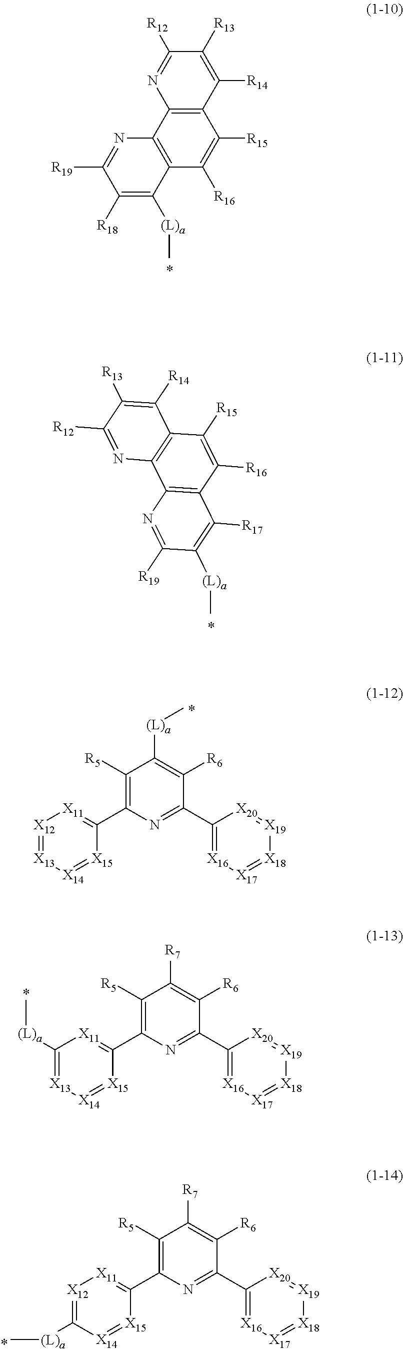 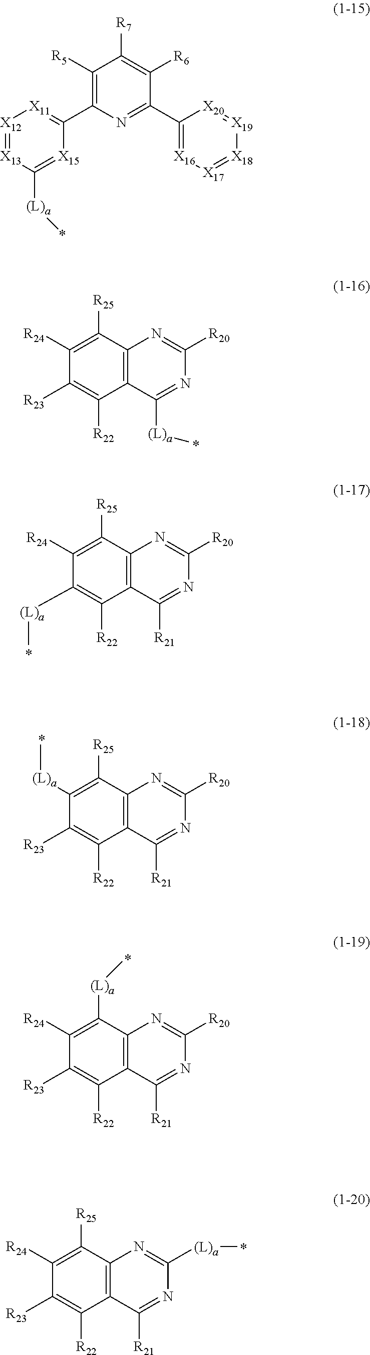 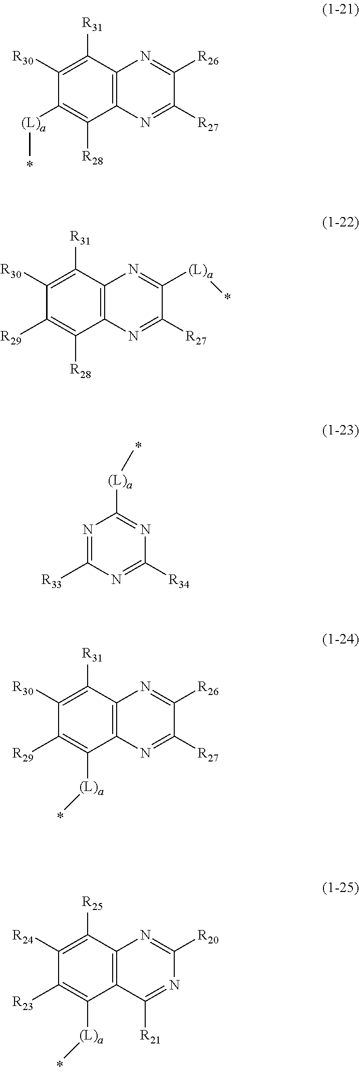
wherein,
X₁₁ to X₂₀ each independently represent CR₈ or N;
R₅ to R₈ and R₁₂ to R₃₄ each independently represent hydrogen, deuterium, a halogen, a cyano, a substituted or unsubstituted (C₁-C₃₀)alkyl, a substituted or unsubstituted (C₃-C₃₀)cycloalkyl, a substituted or unsubstituted (C₆-C₃₀)aryl, or a substituted or unsubstituted (3- to 30-membered)heteroaryl; and
L and a are as defined in claim 1.

5. The organic electroluminescent compound according to claim 1, wherein the compound represented by Formula 1 is selected from the following compounds: 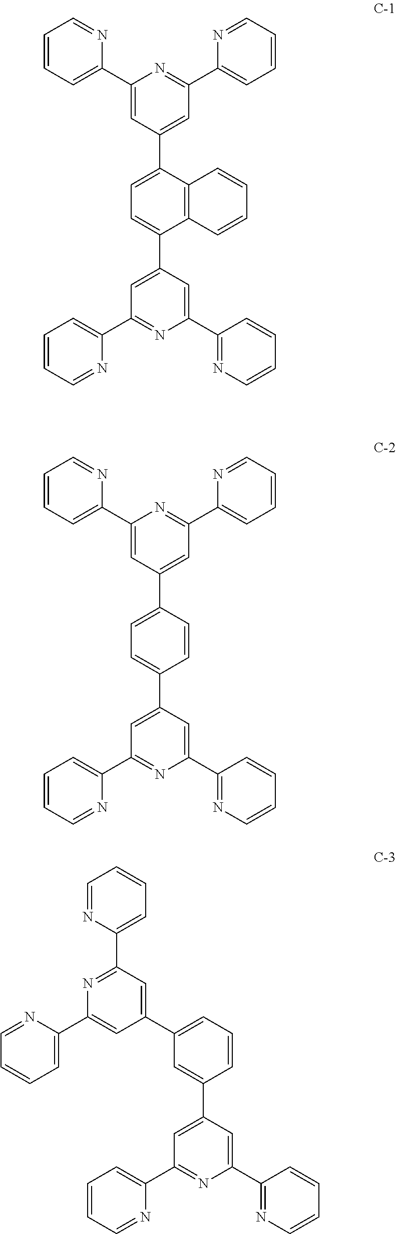 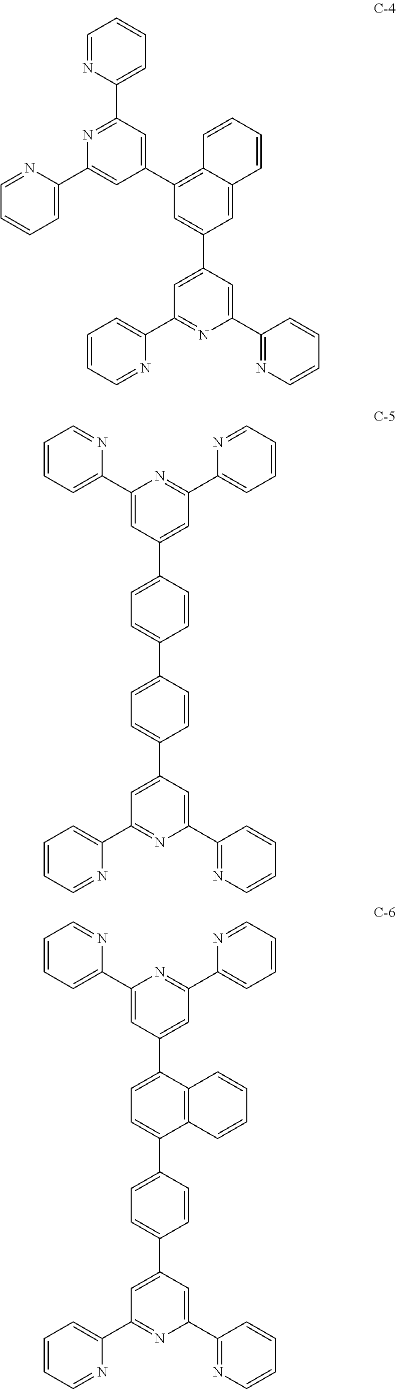 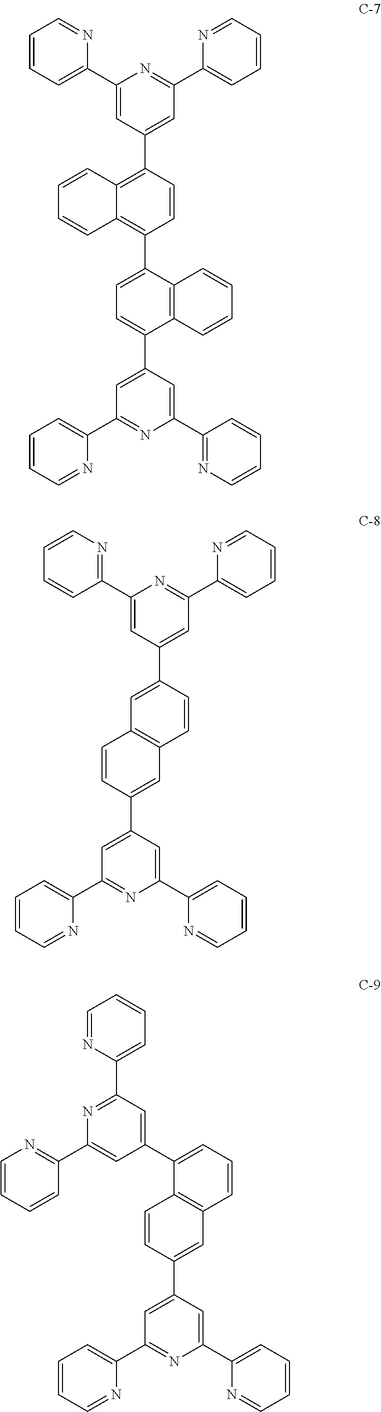 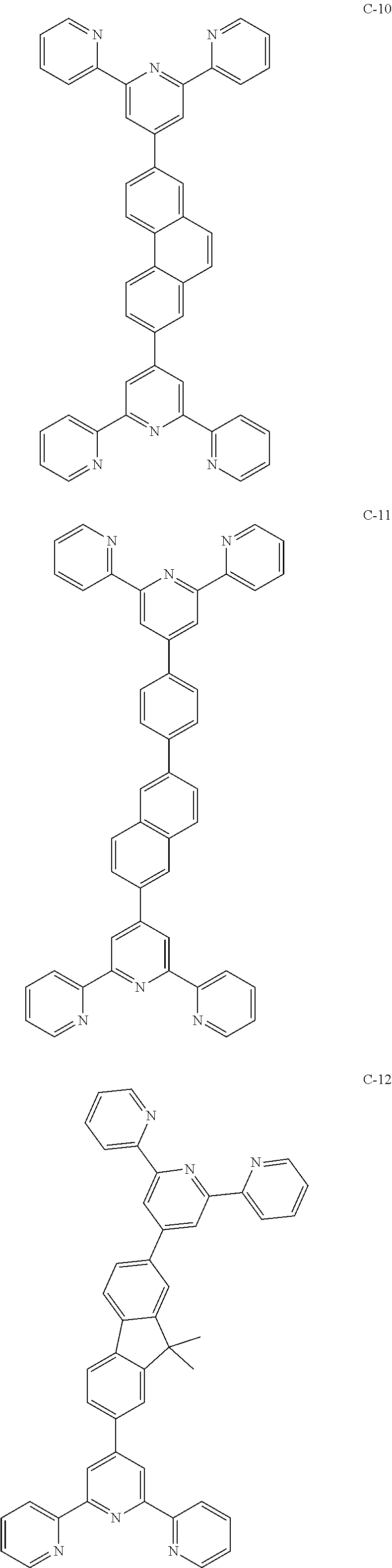 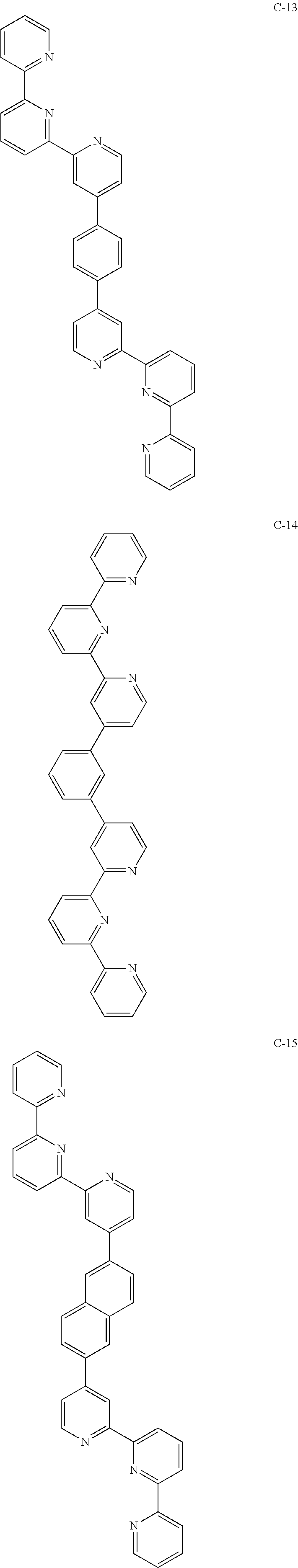 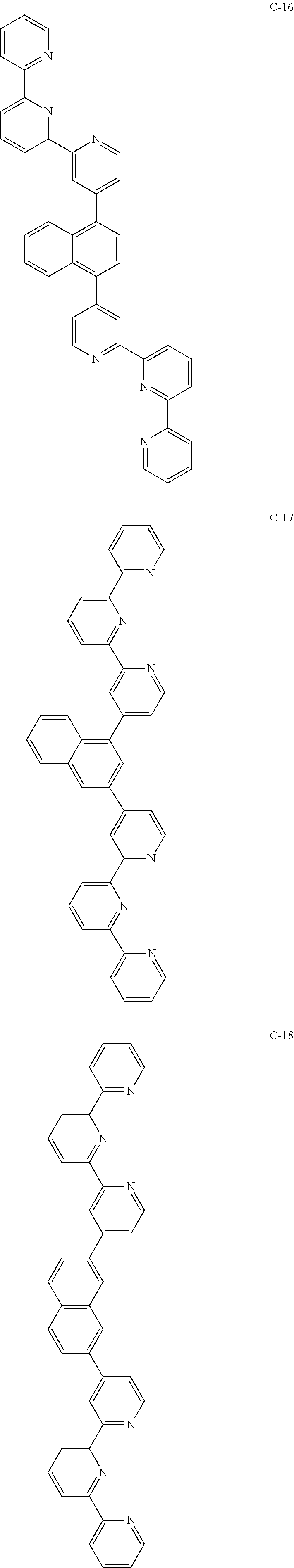 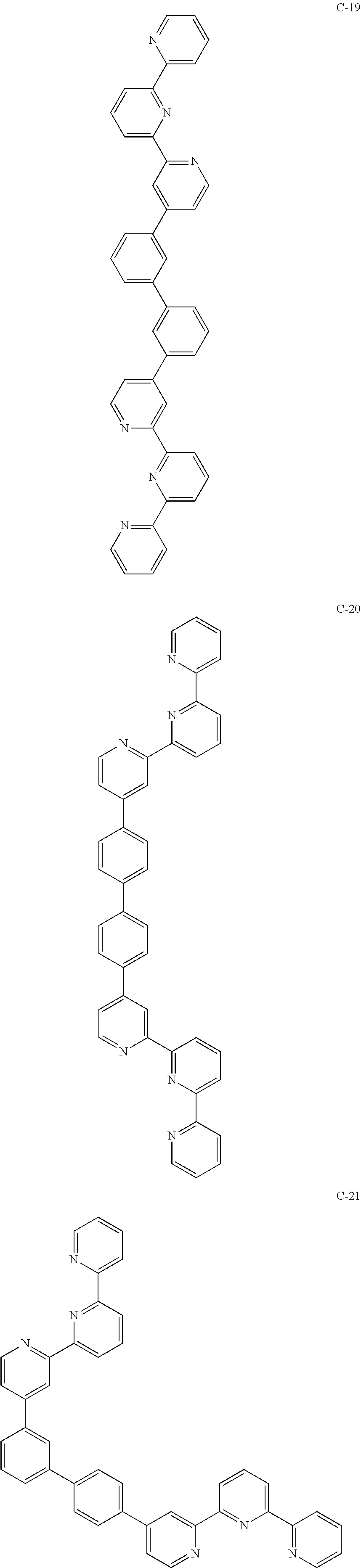 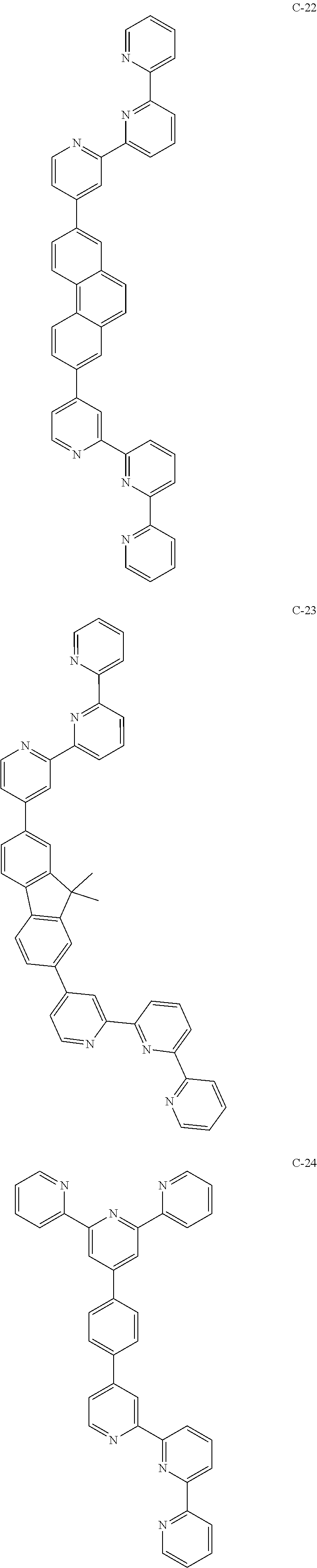 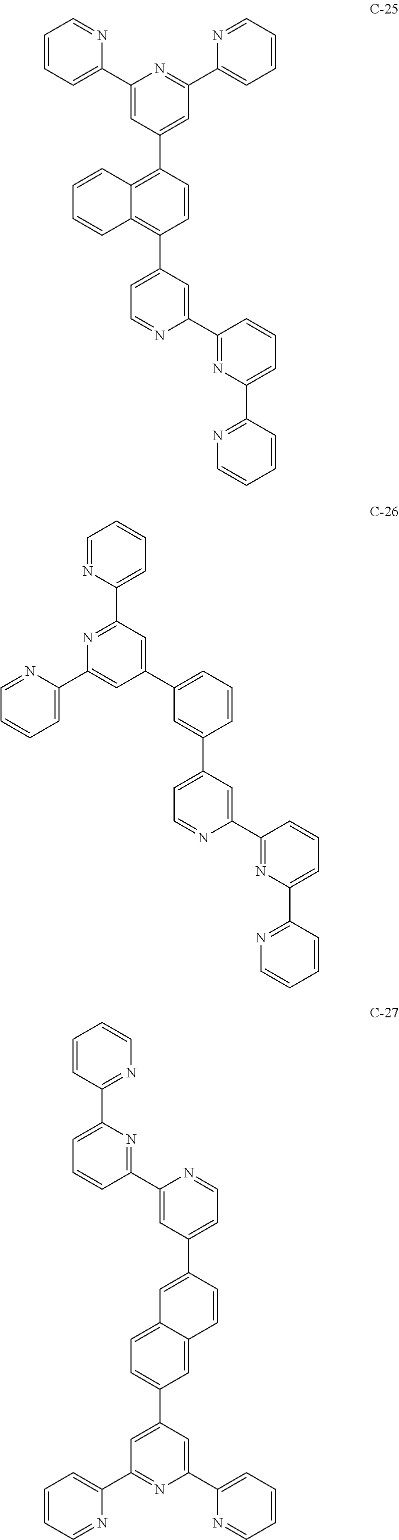 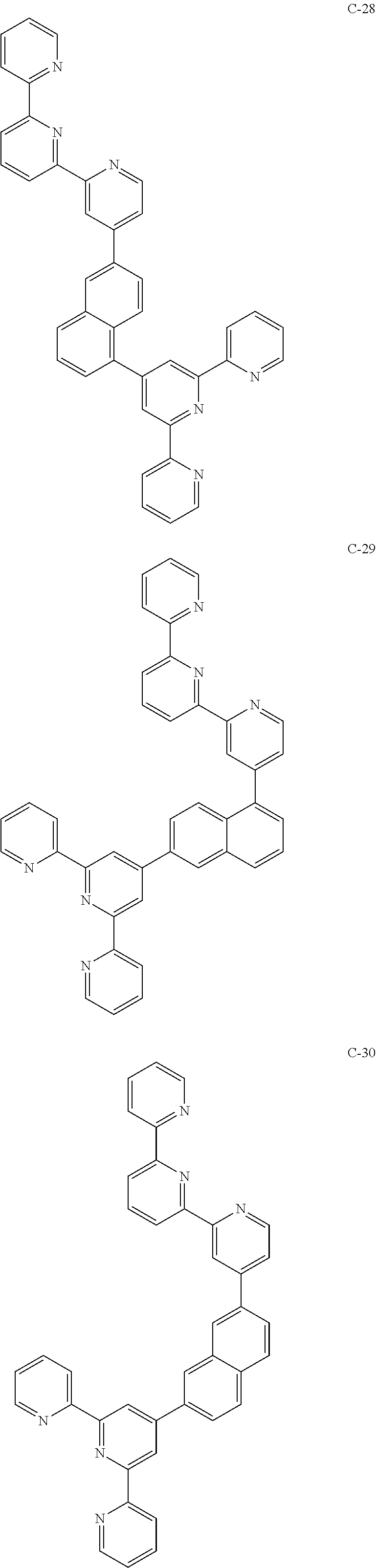 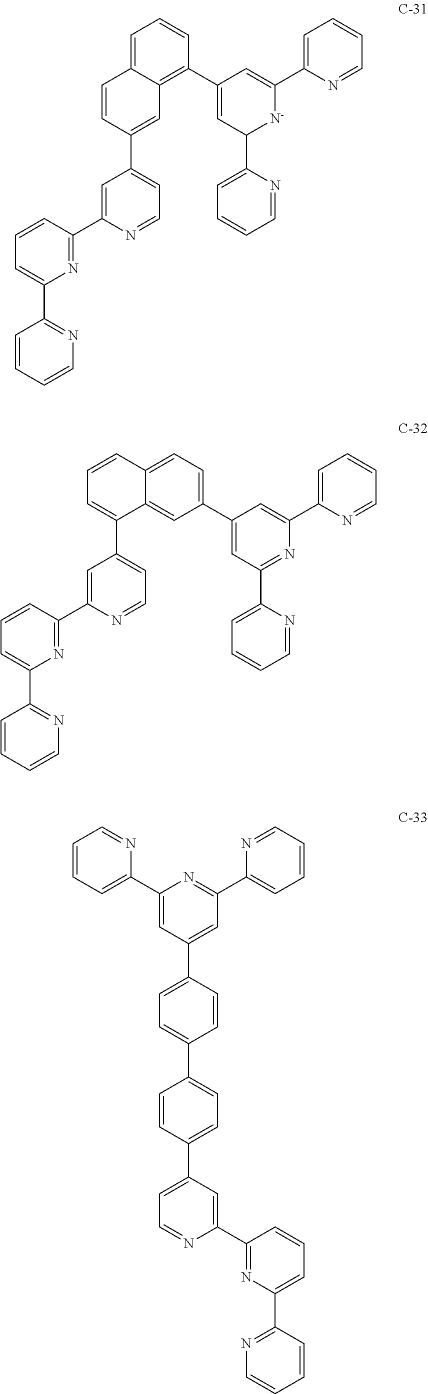 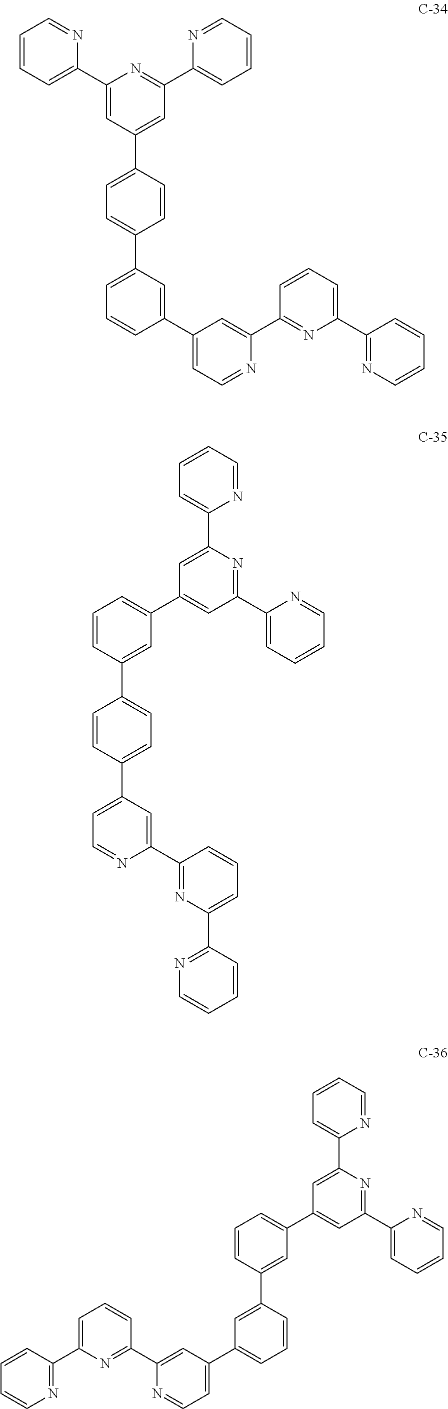 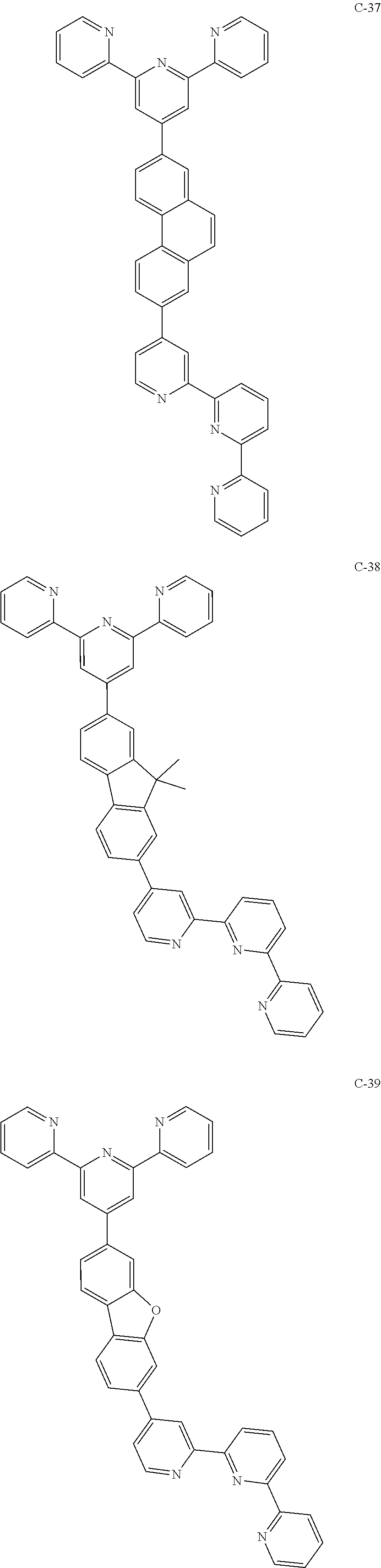 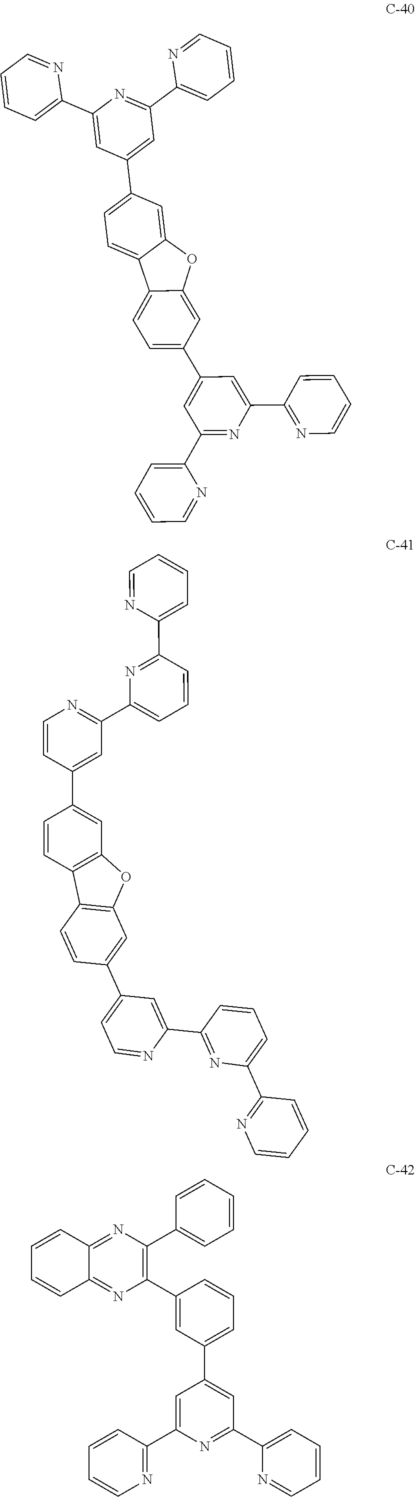 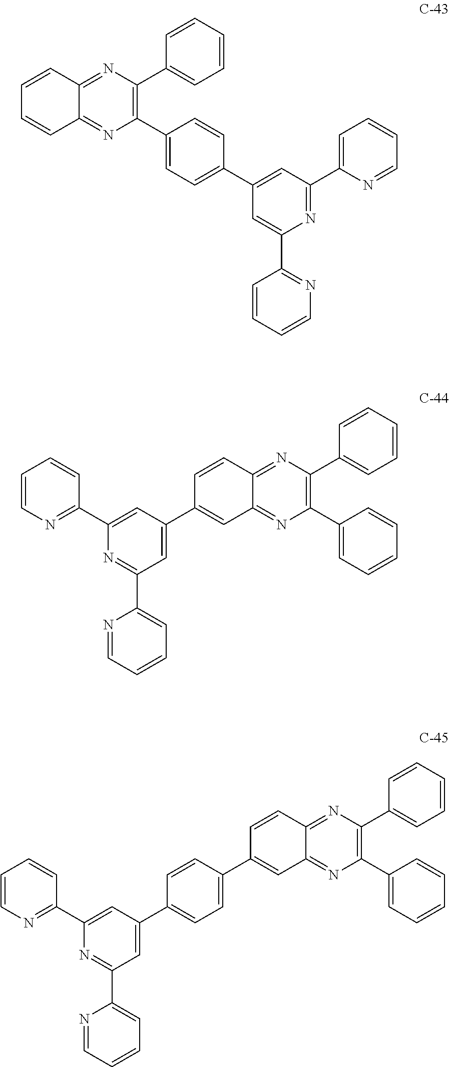 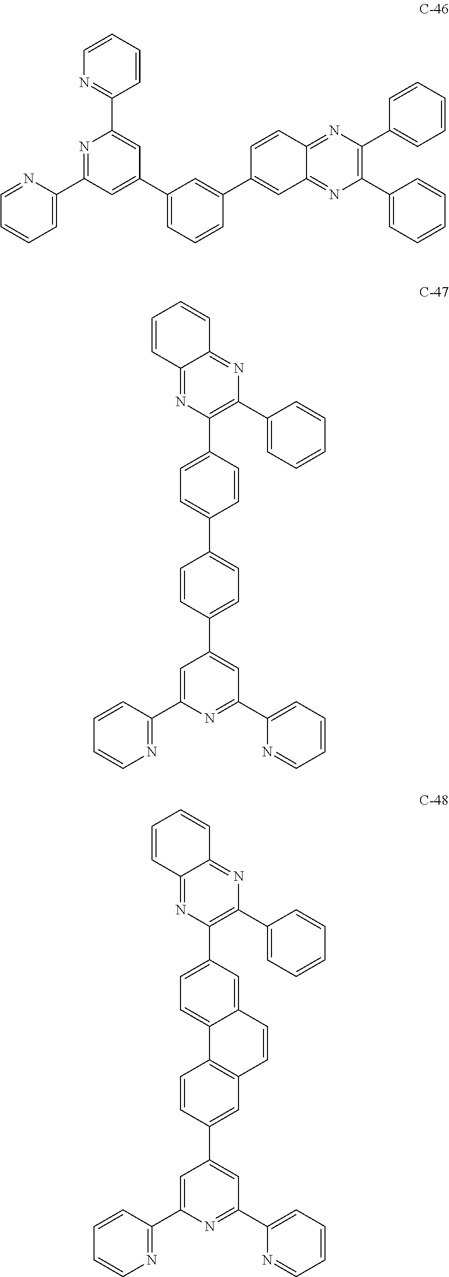 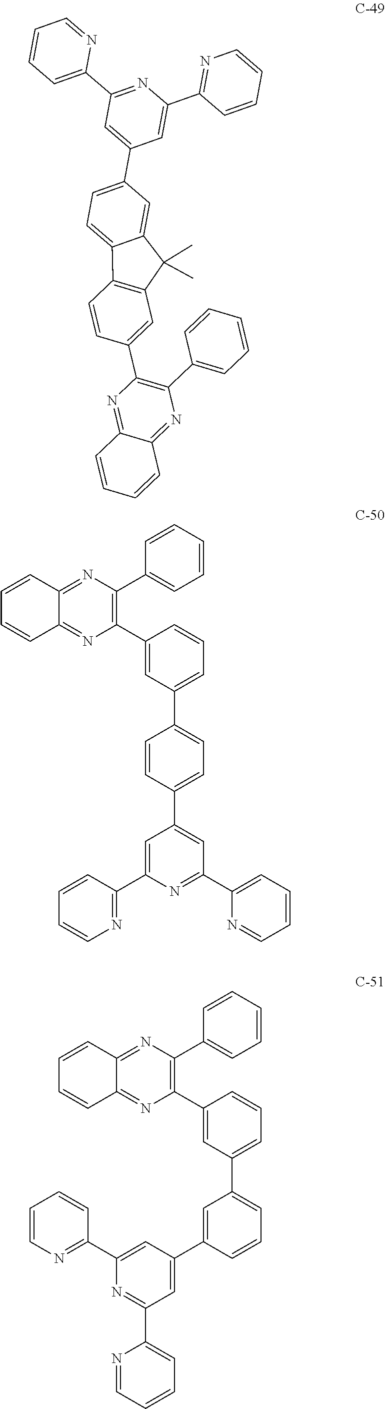 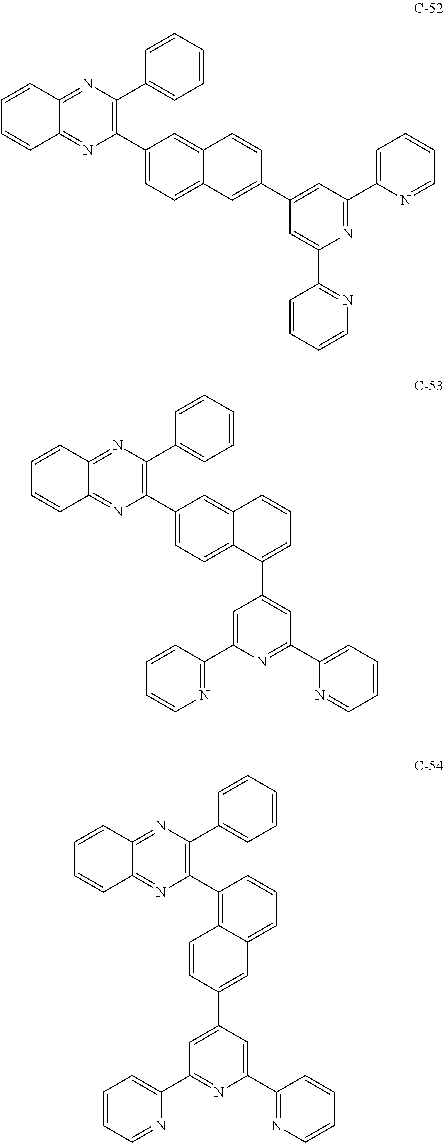 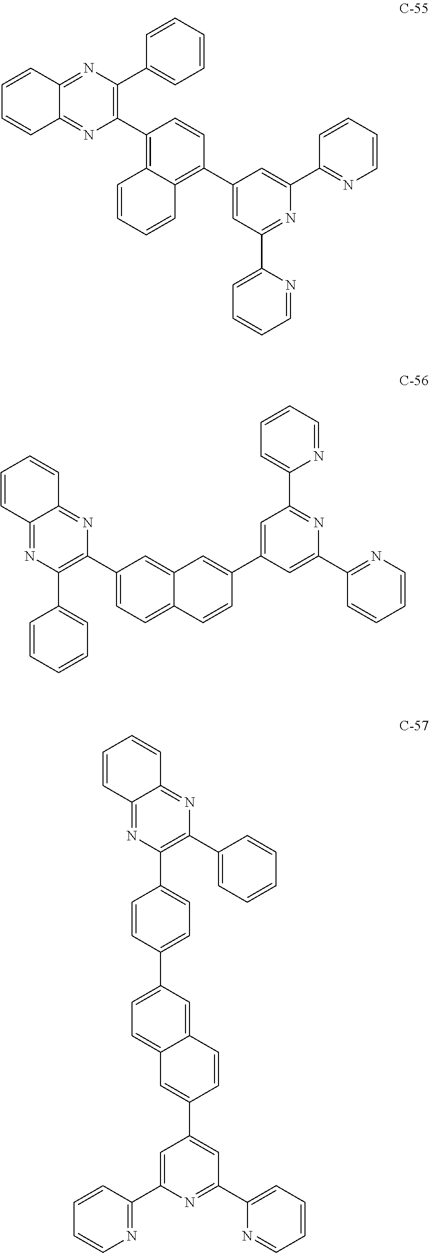 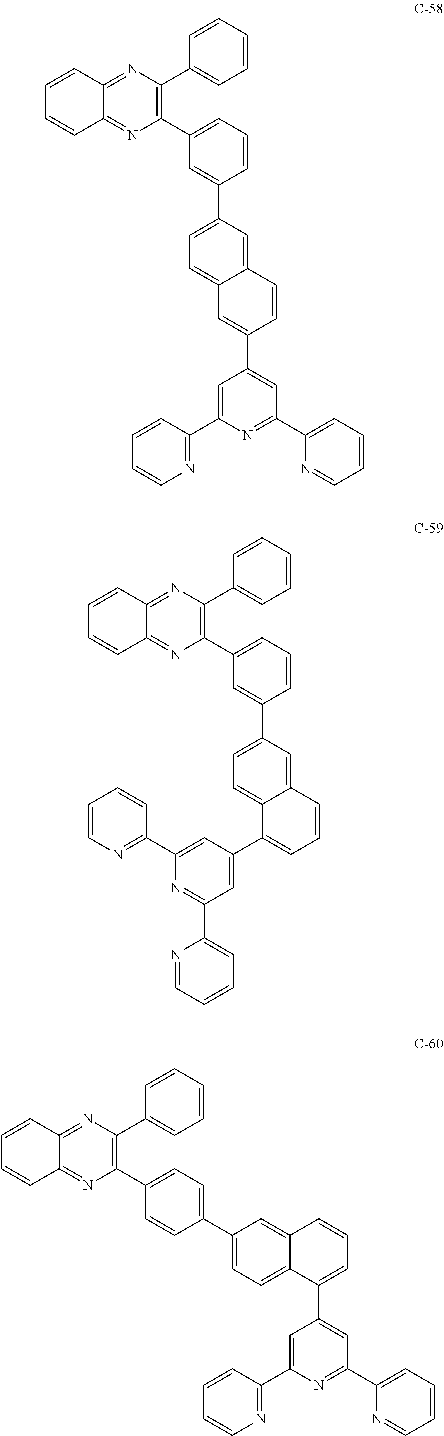 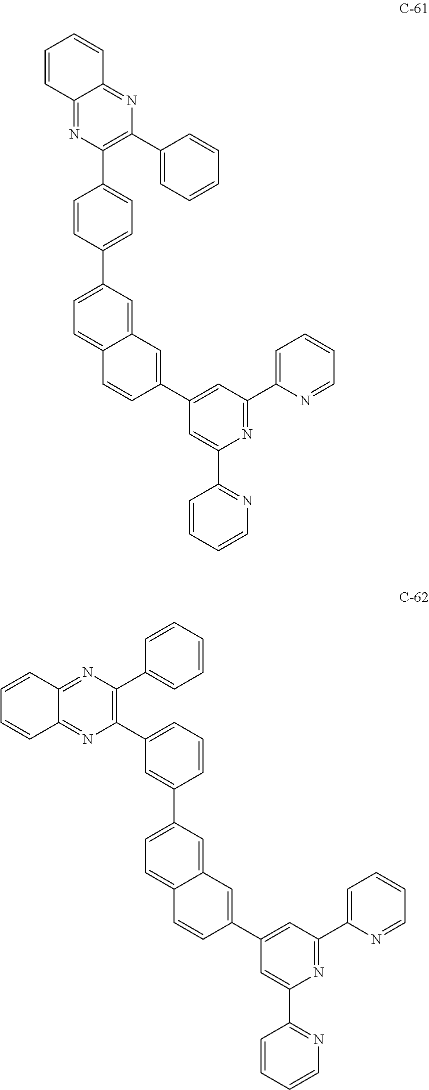 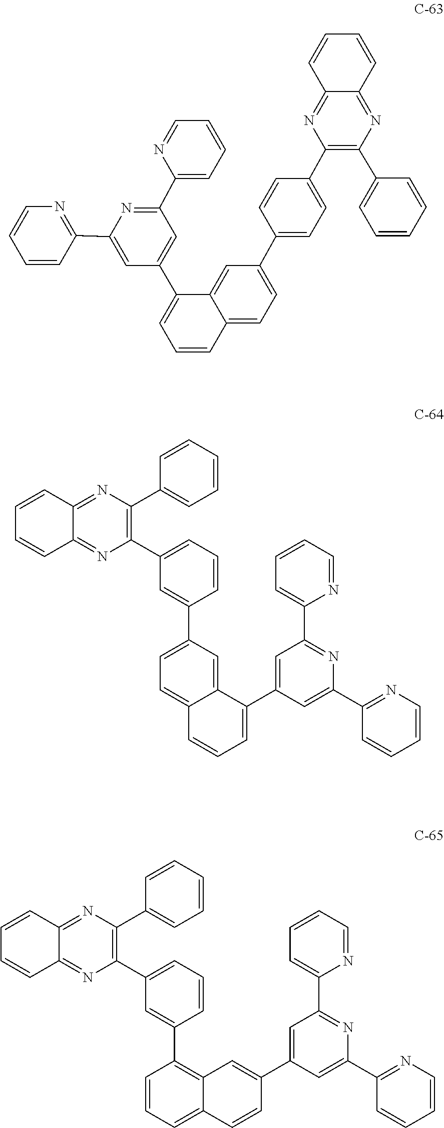 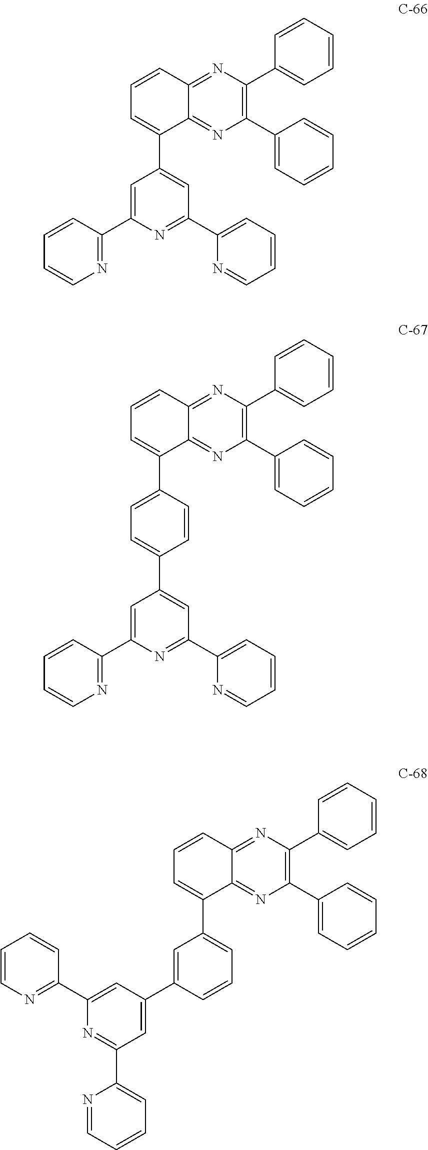 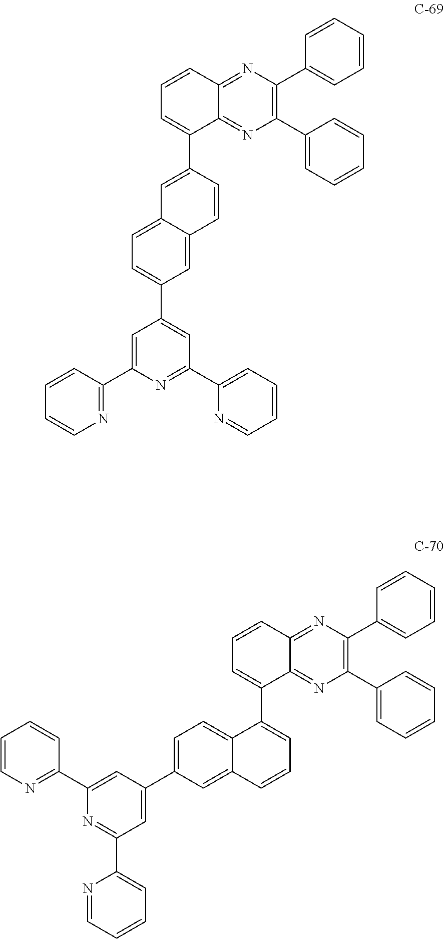 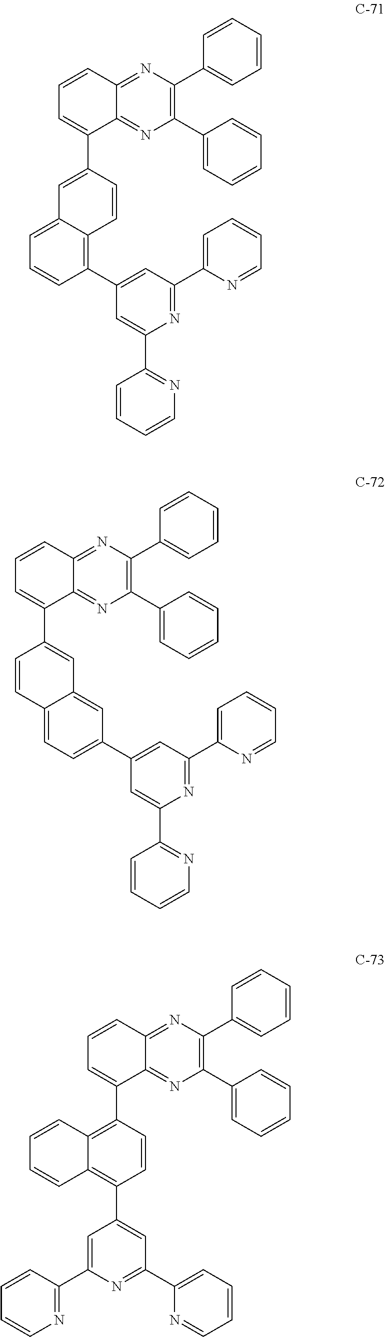 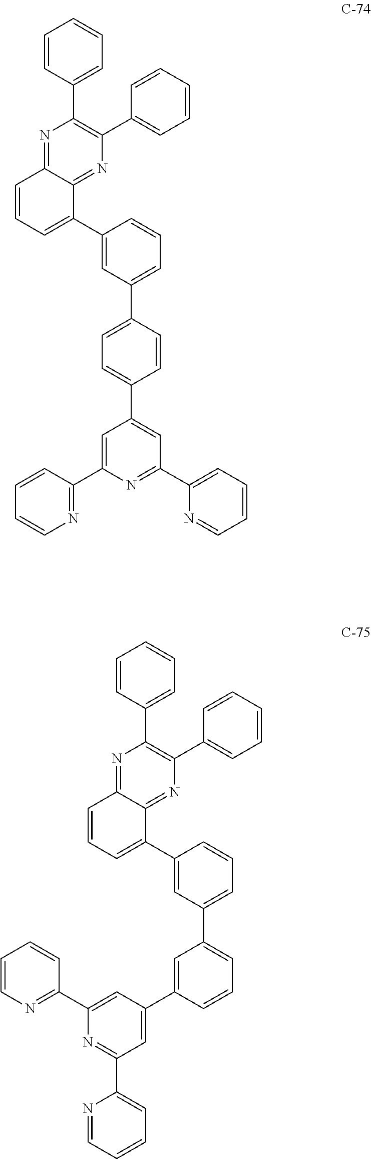 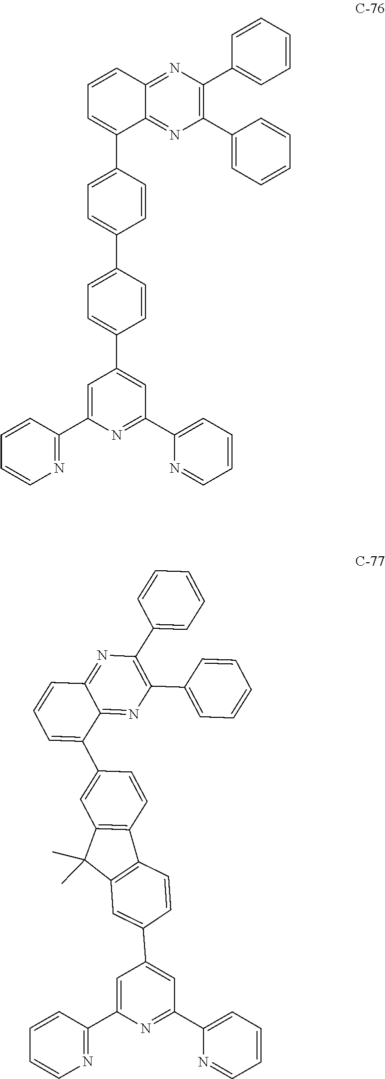 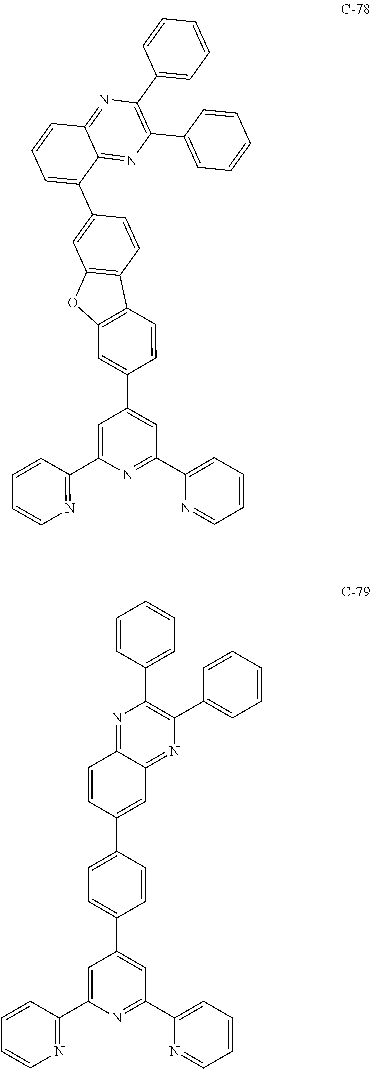 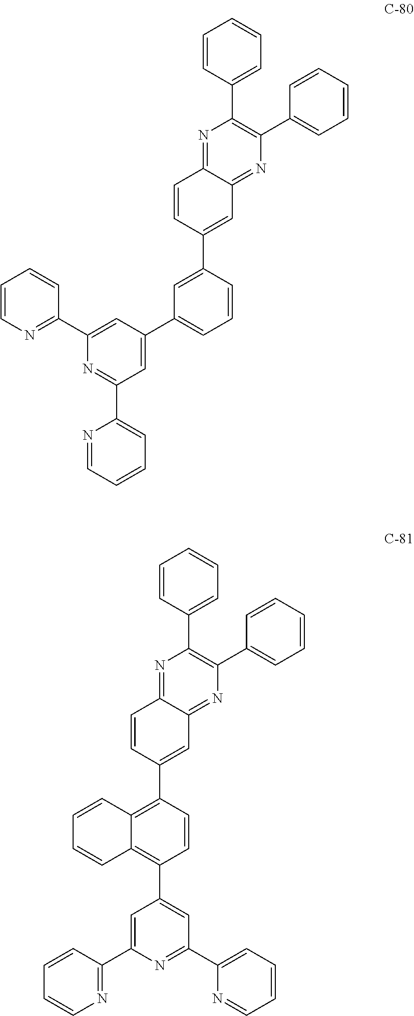 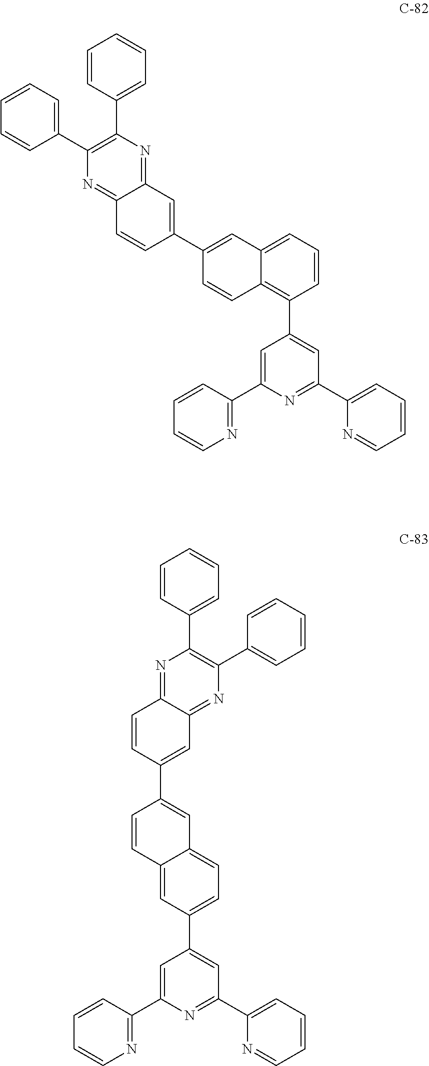 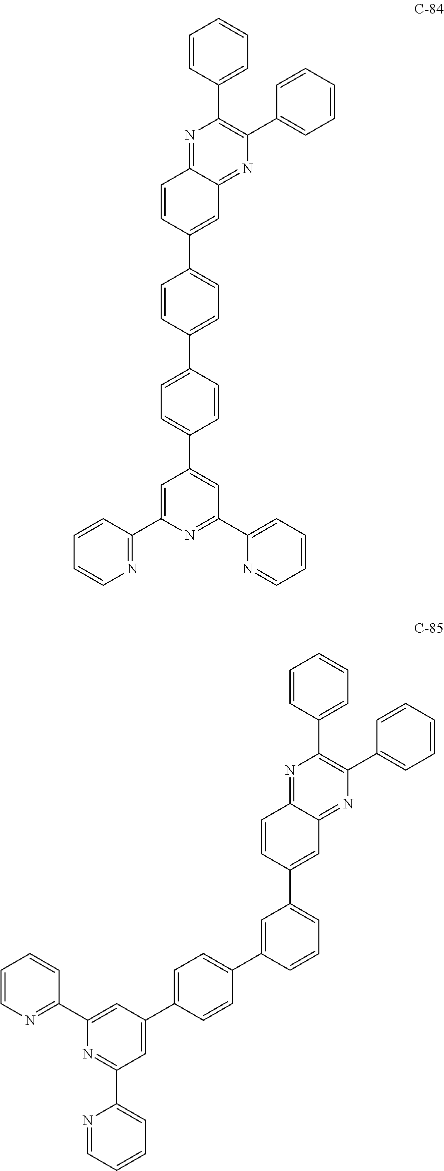 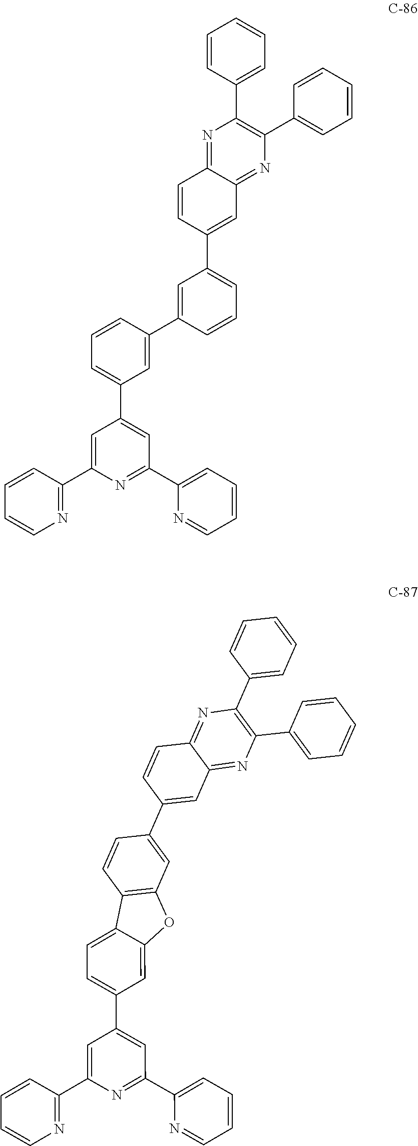 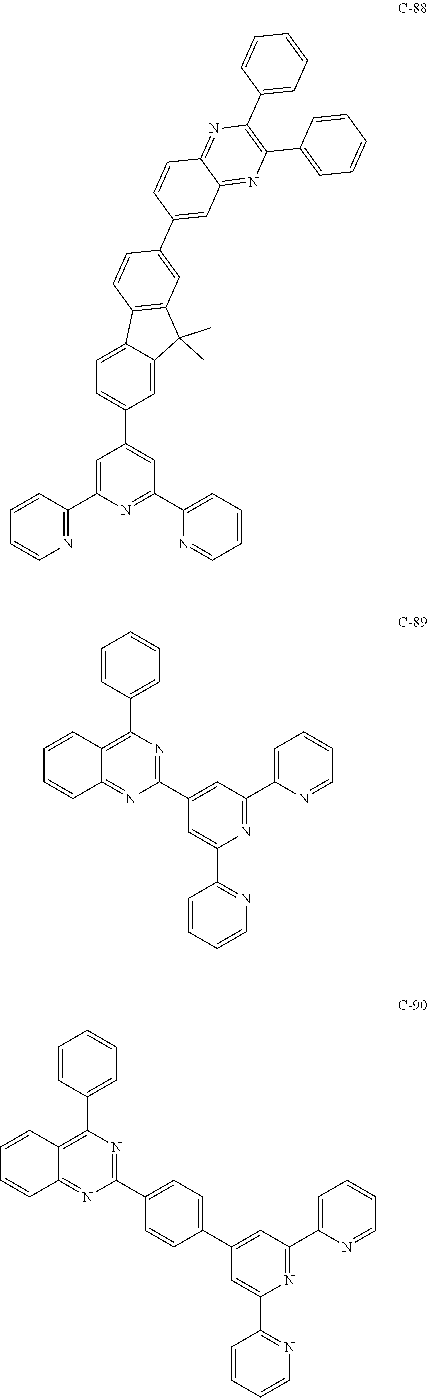 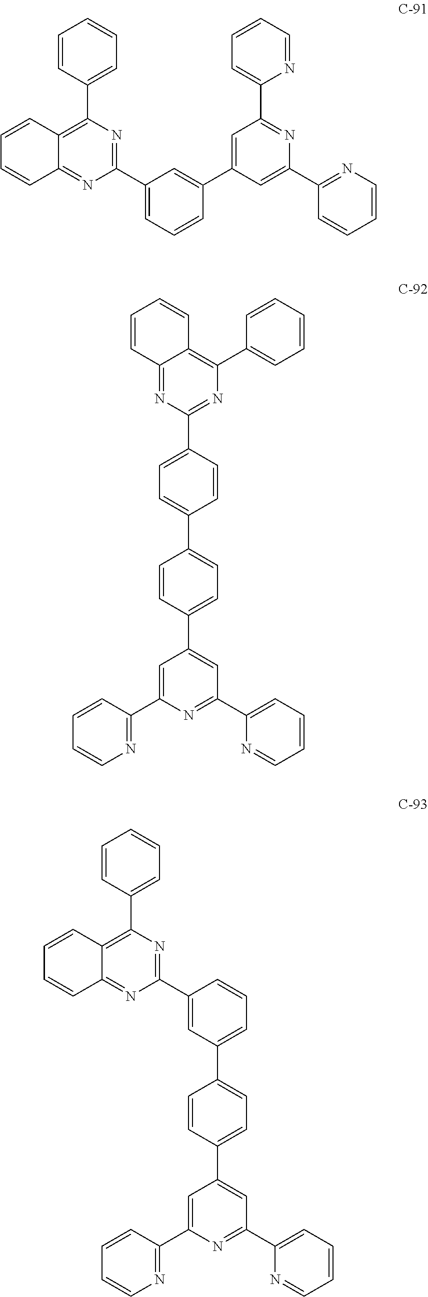 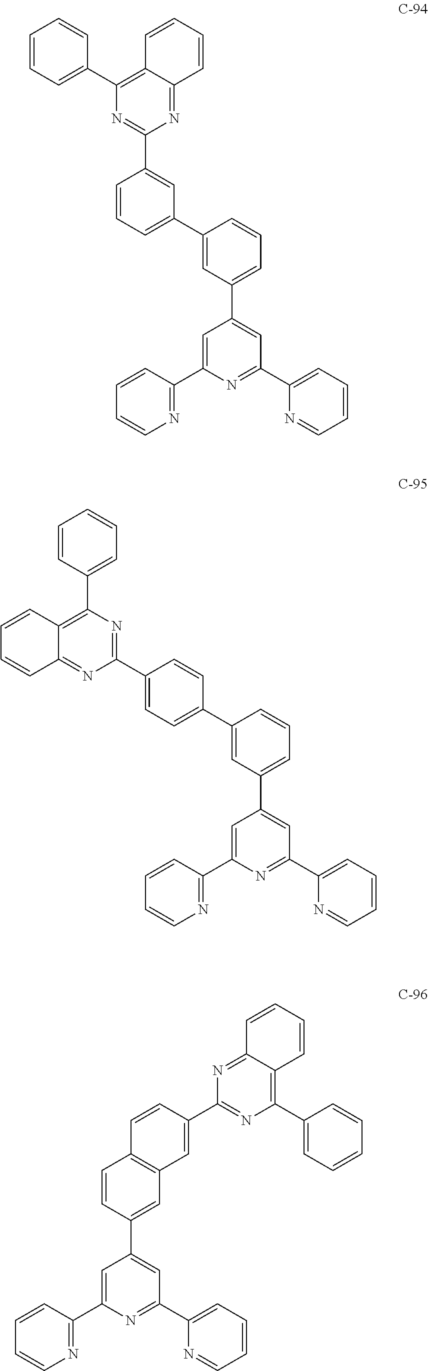 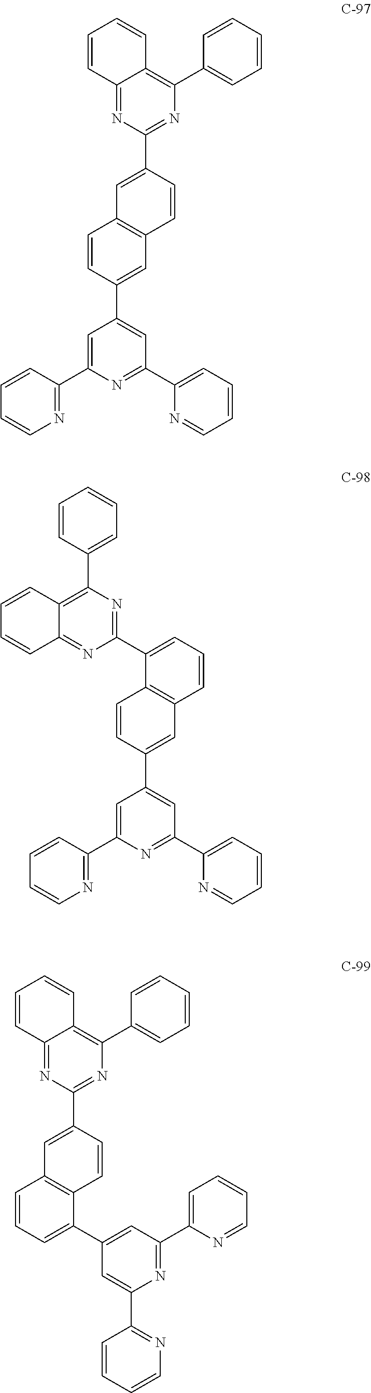 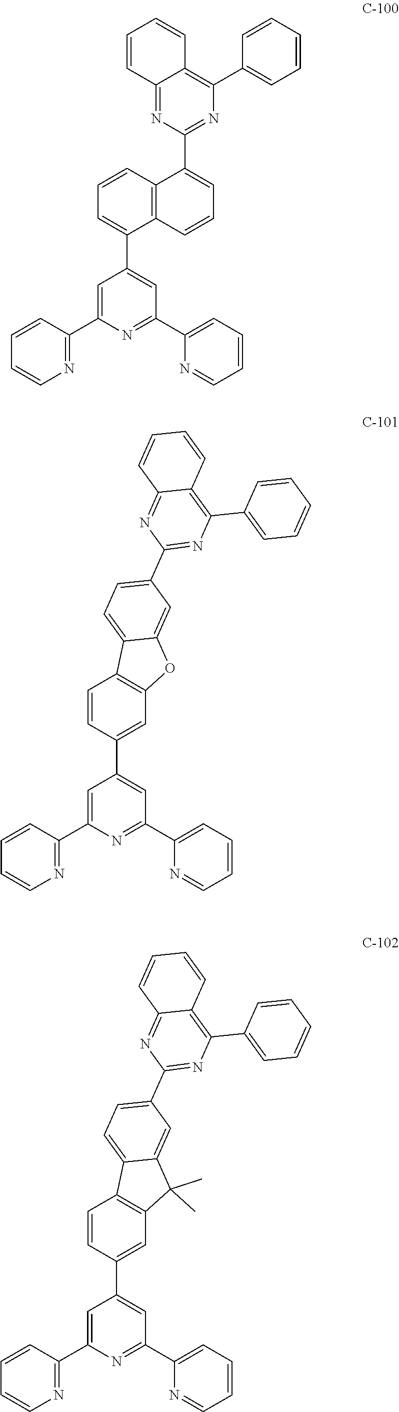 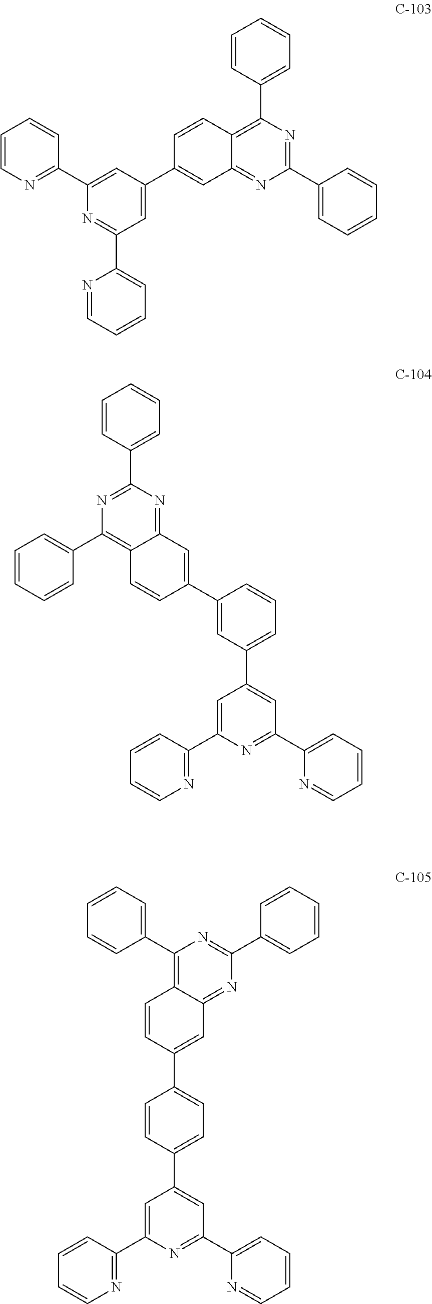 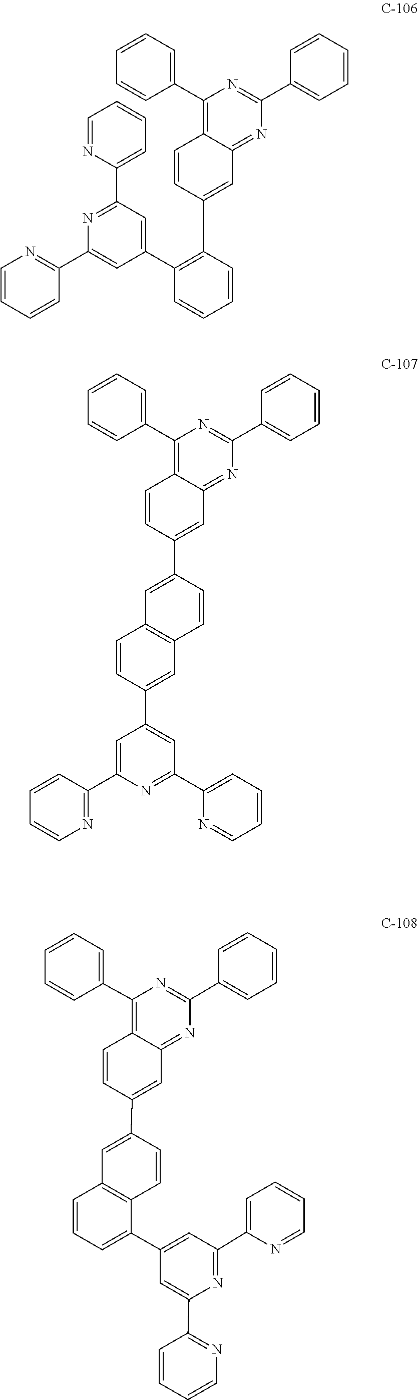 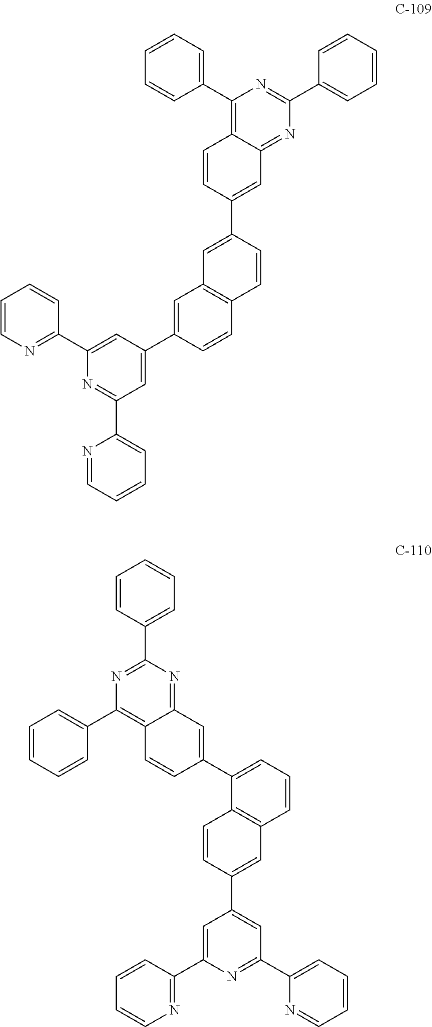 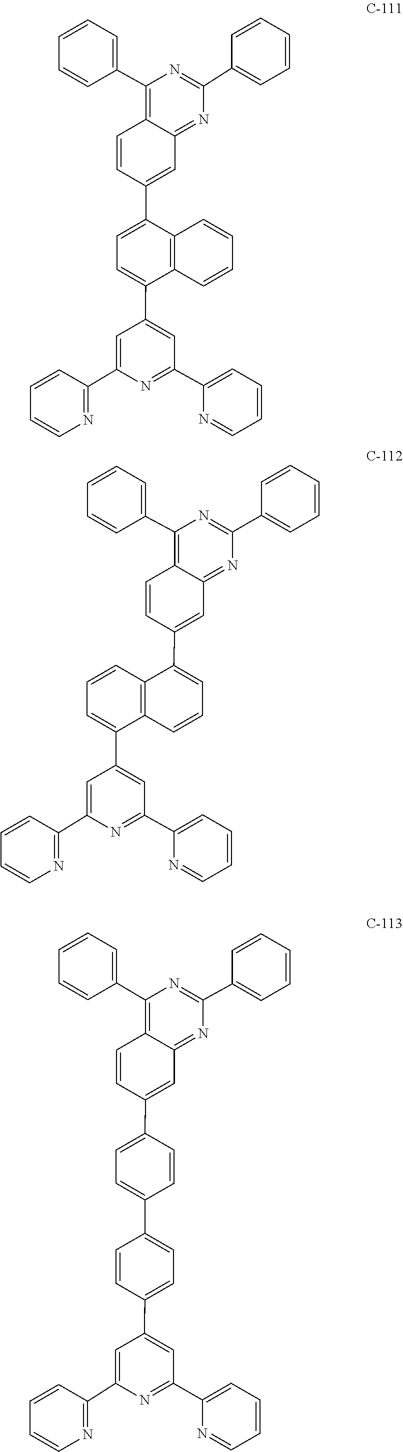 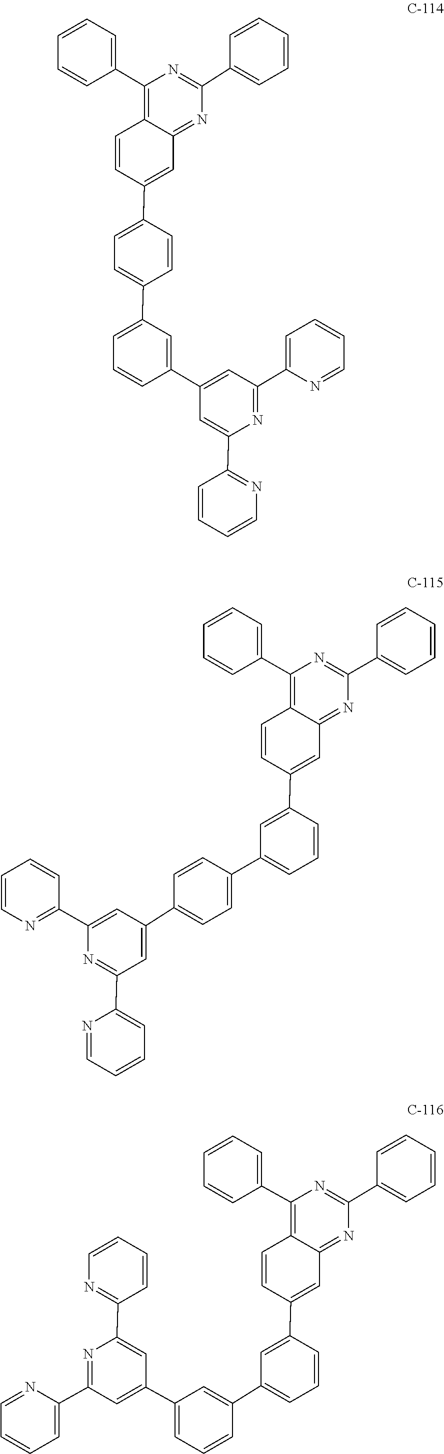 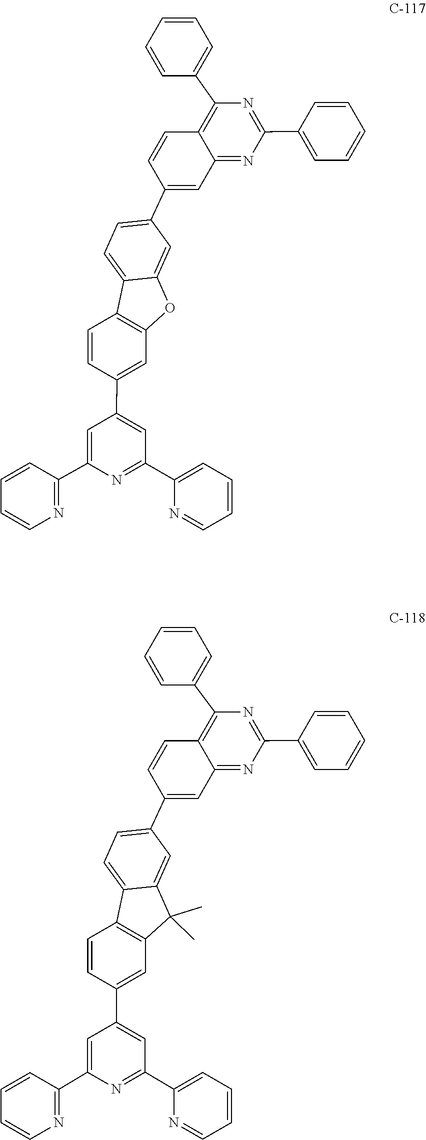 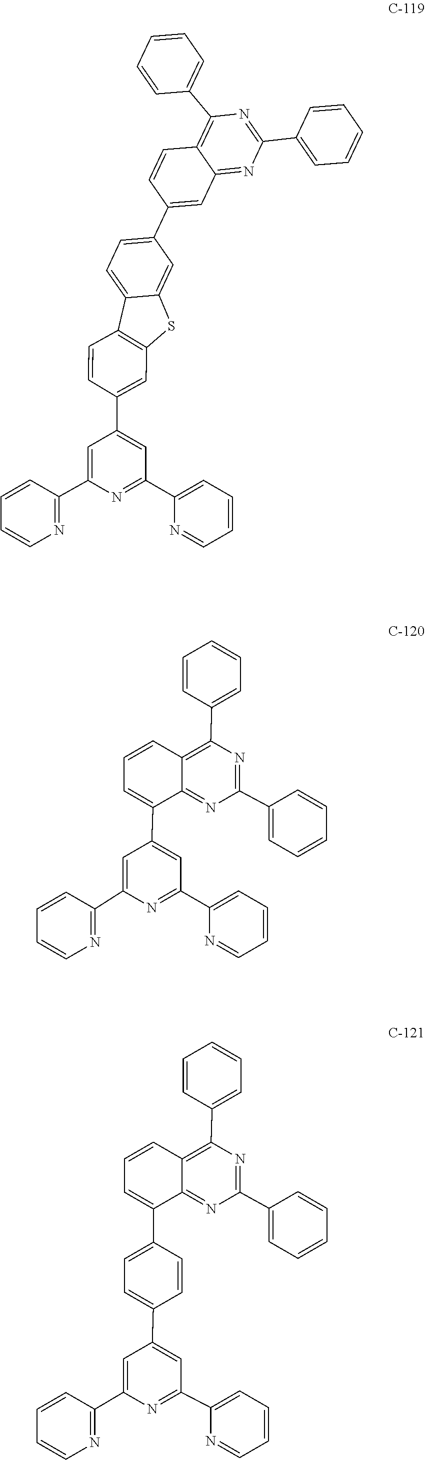 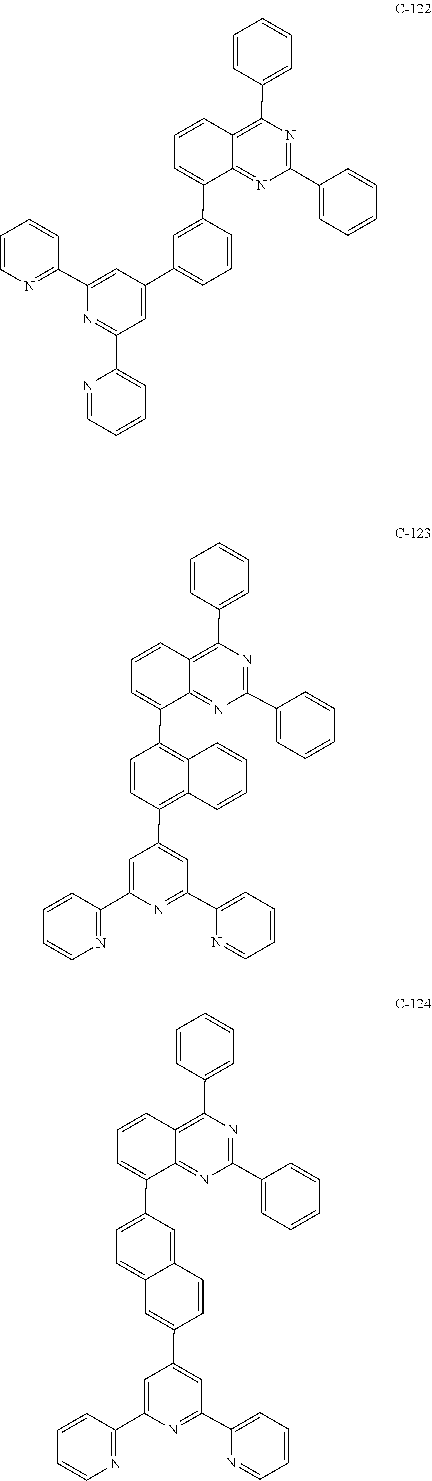 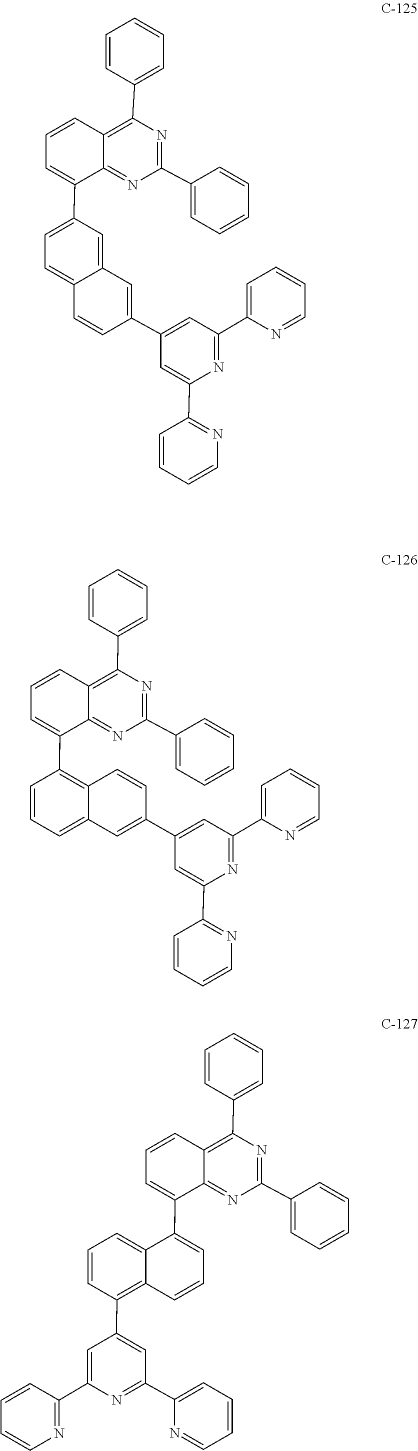 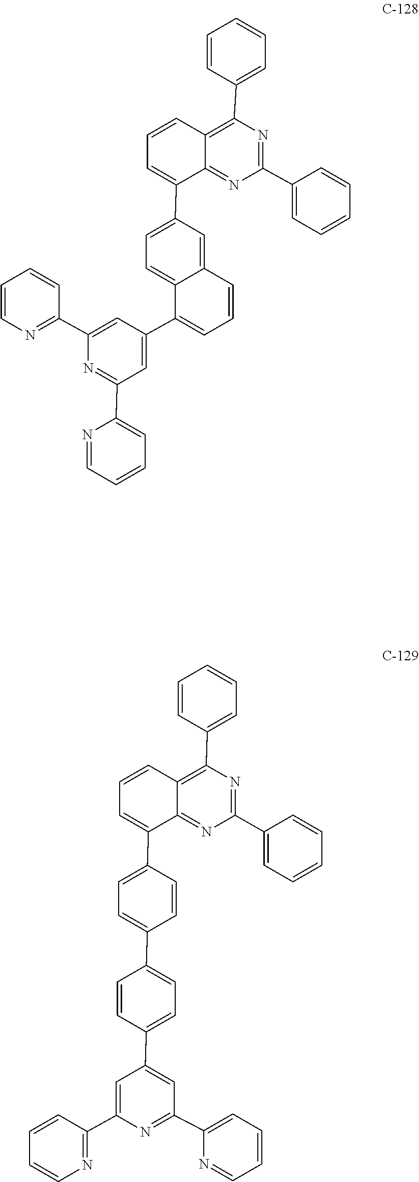 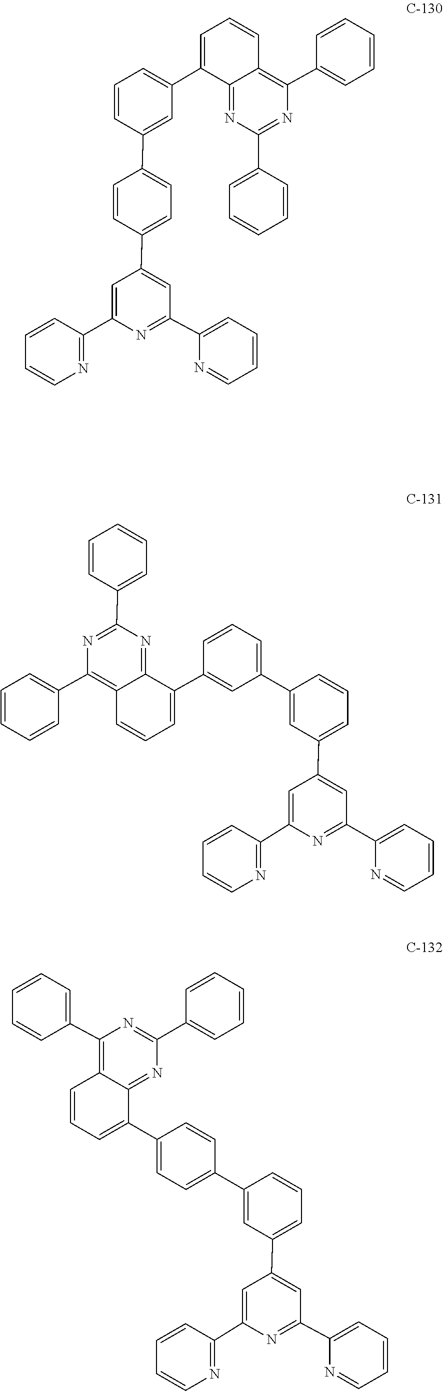 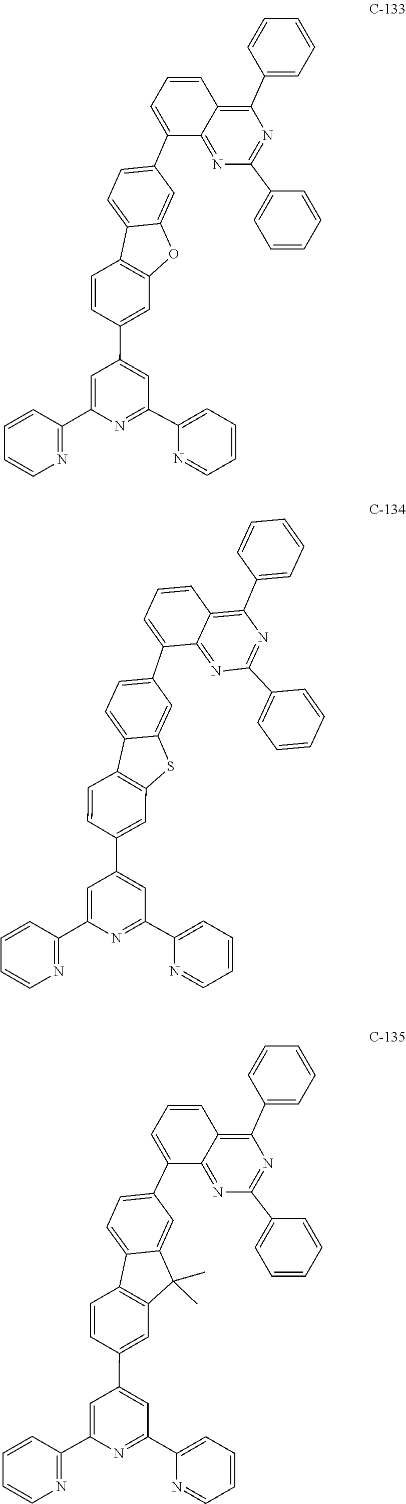 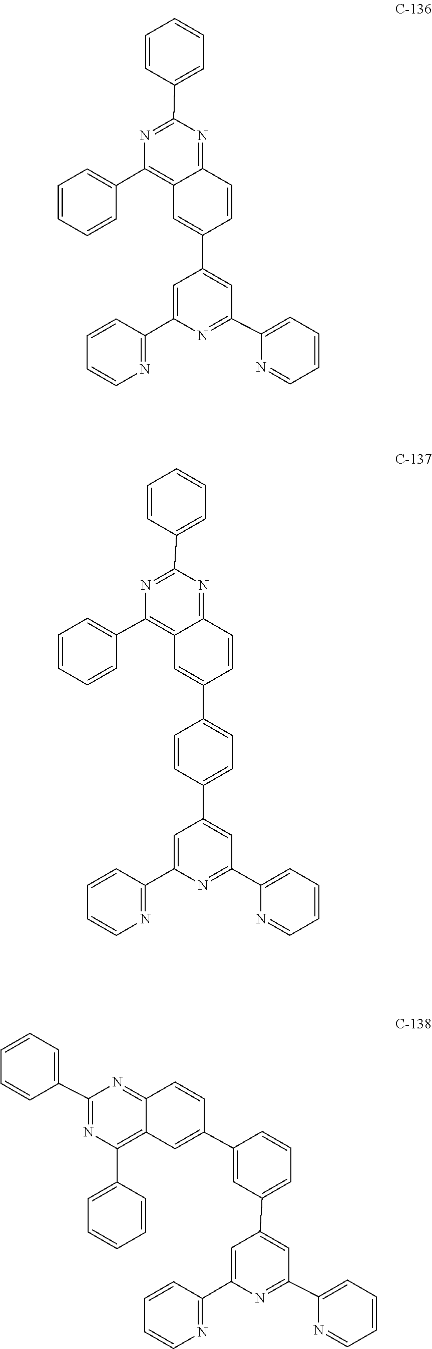 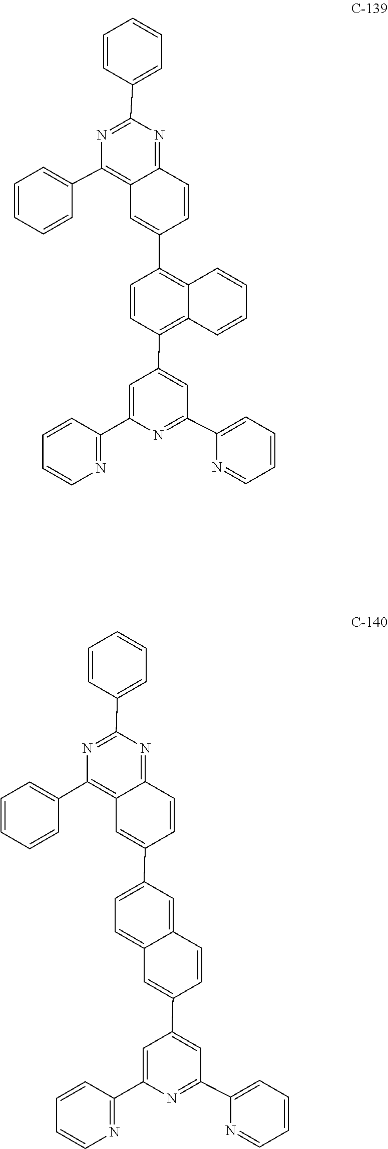 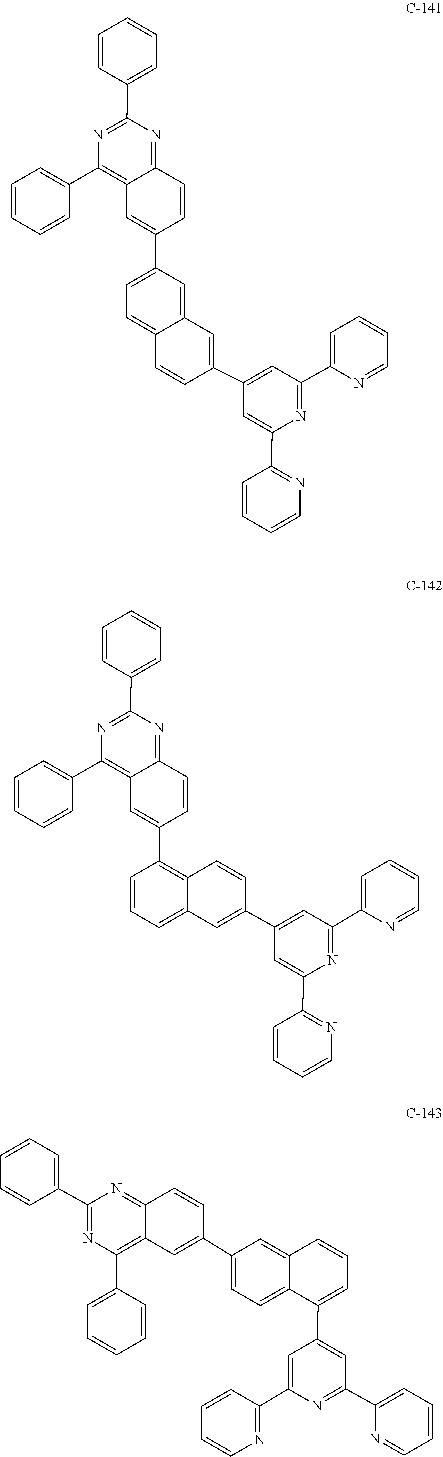 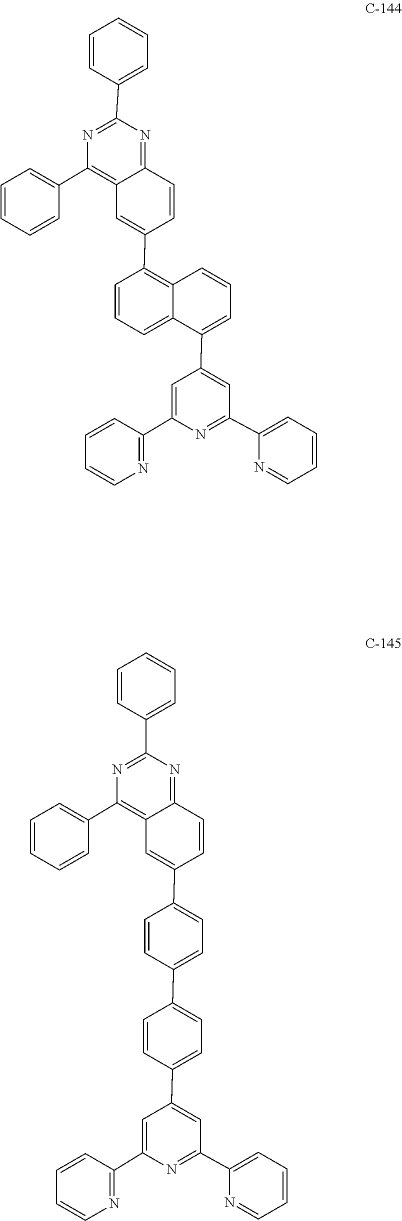 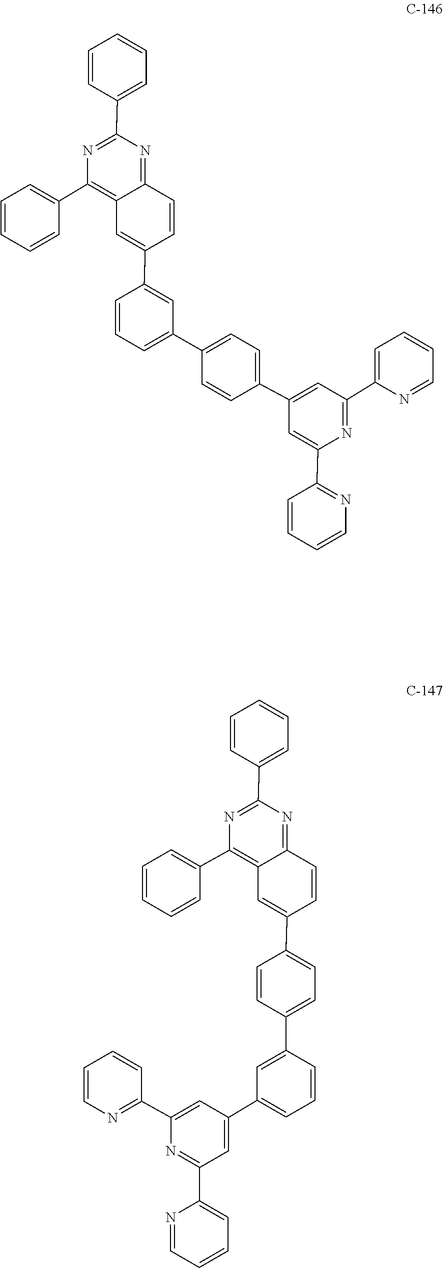 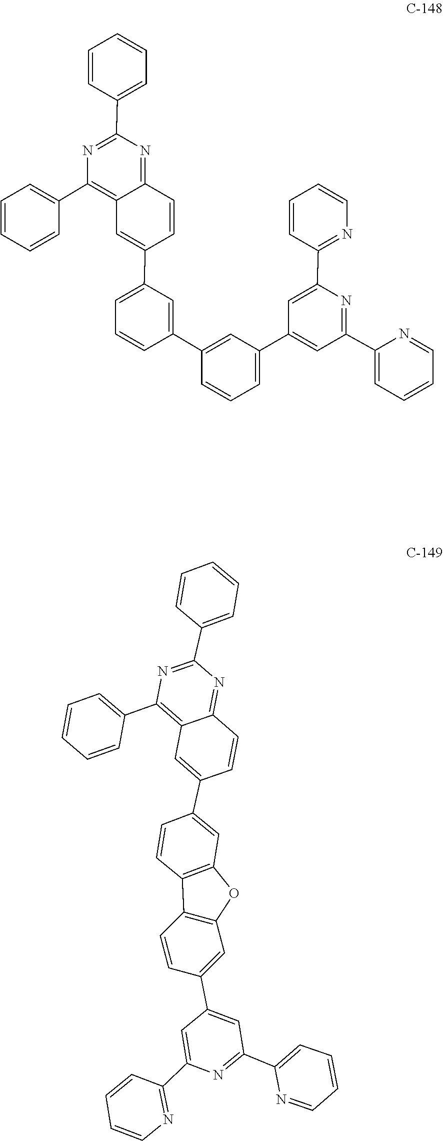 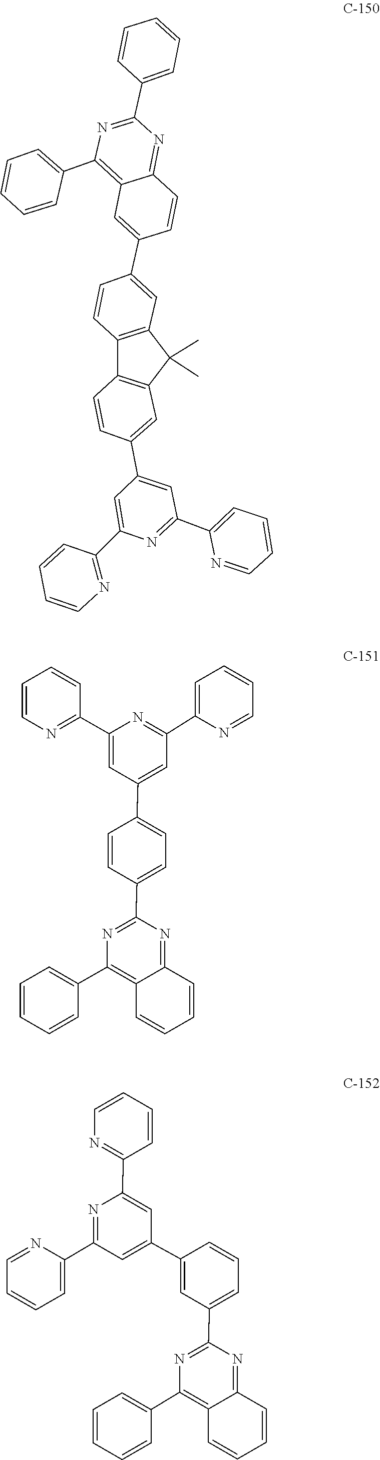 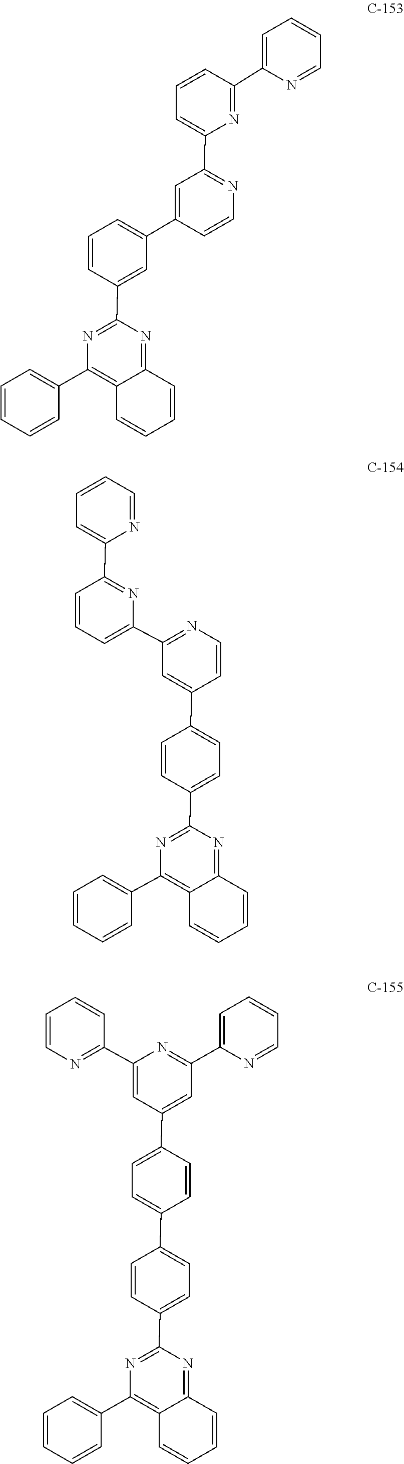 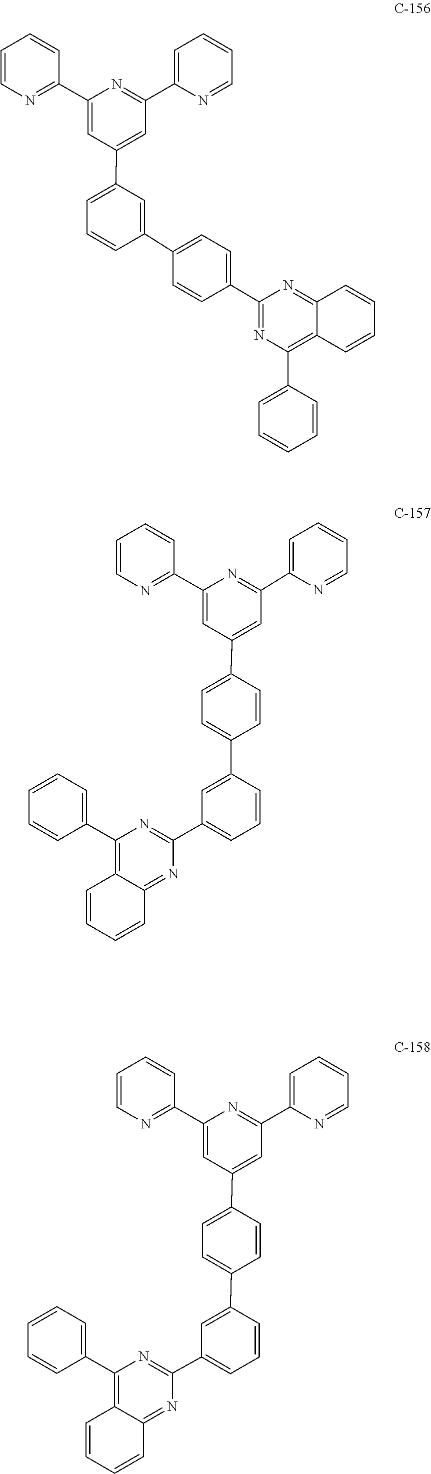 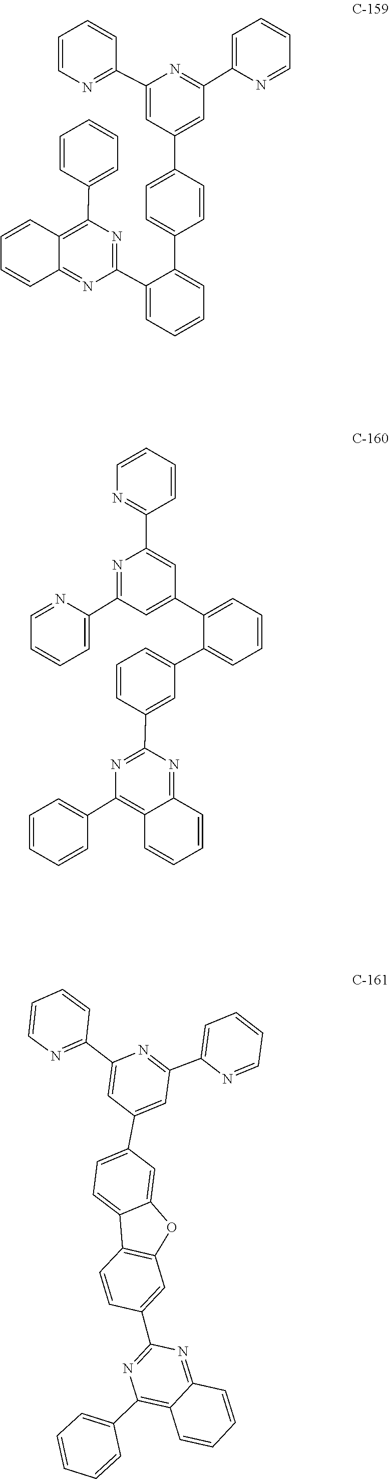 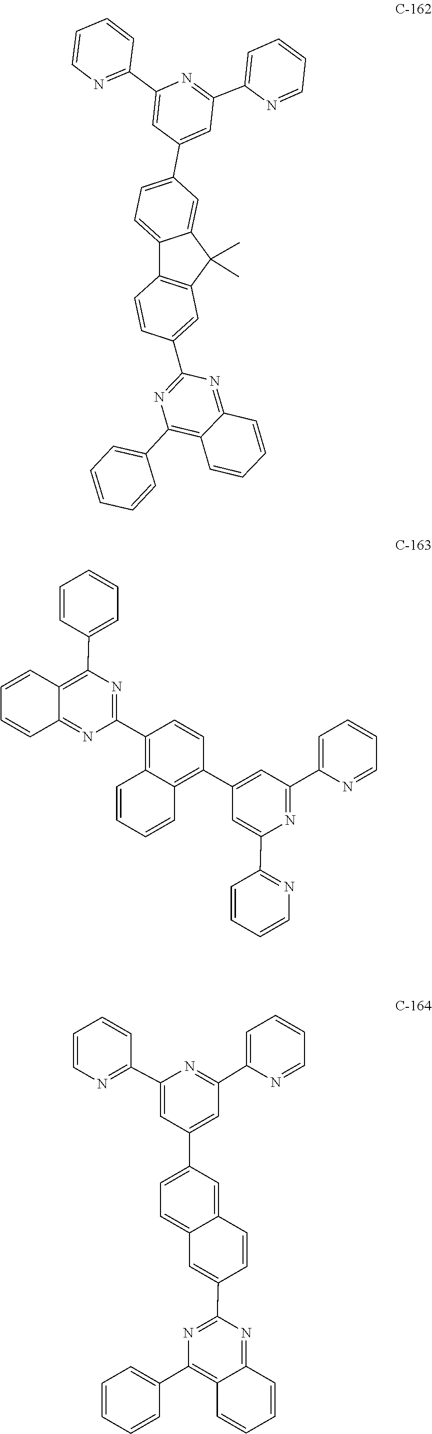 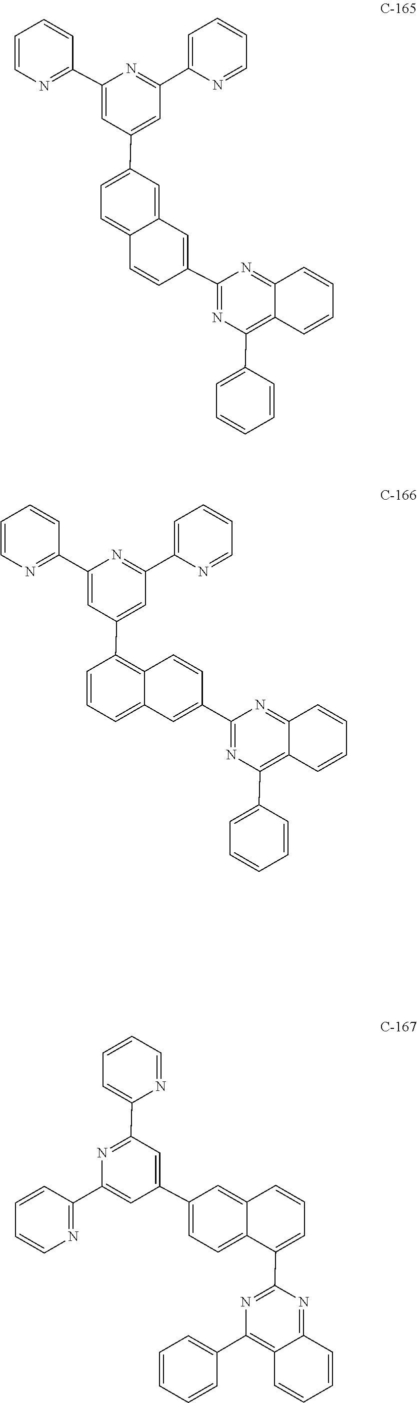 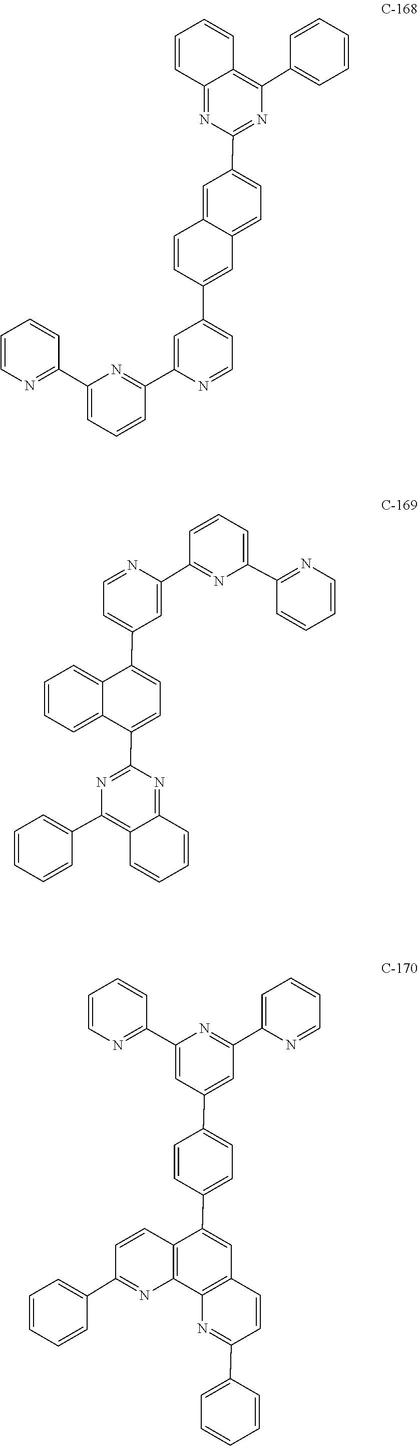 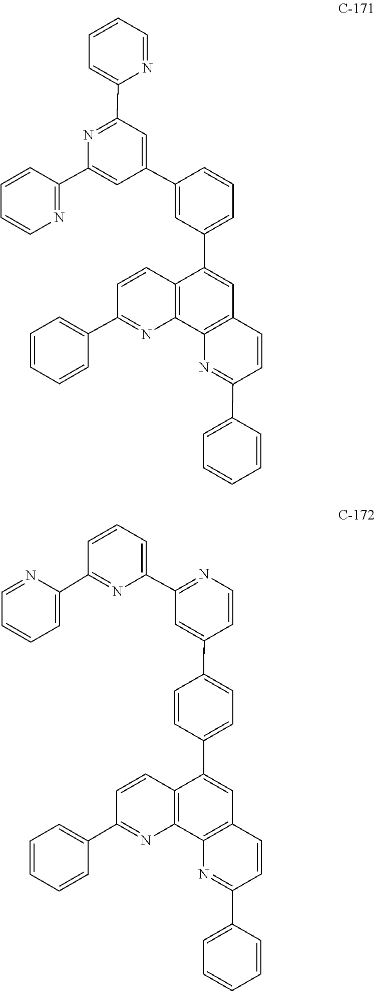 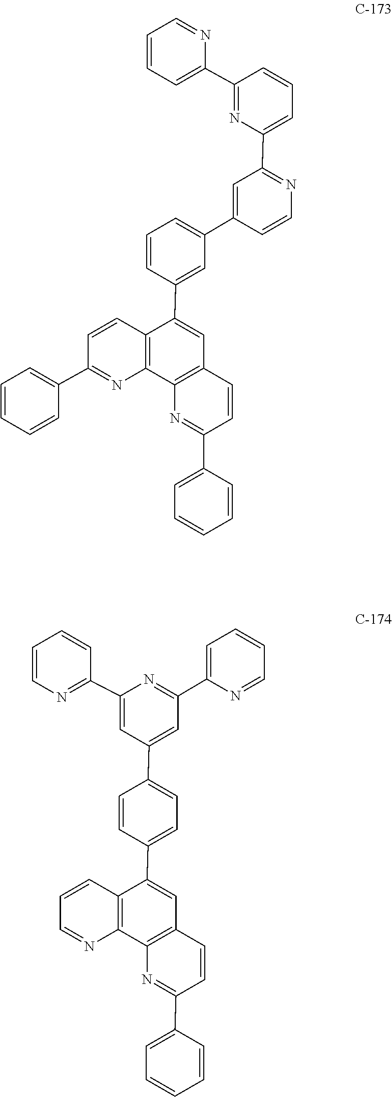 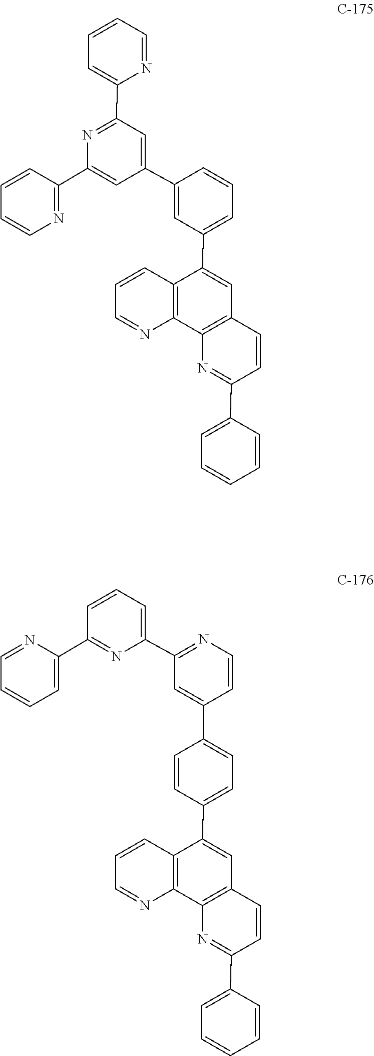 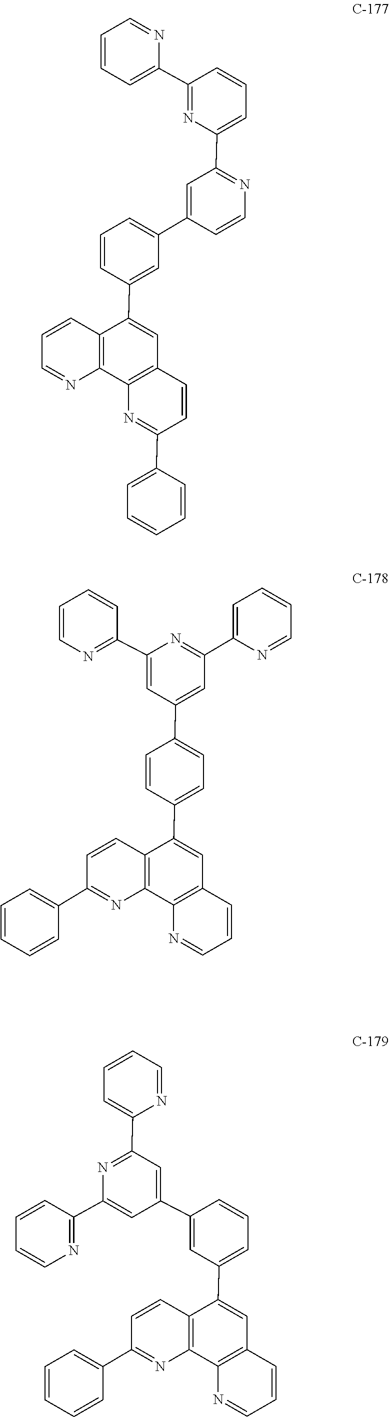 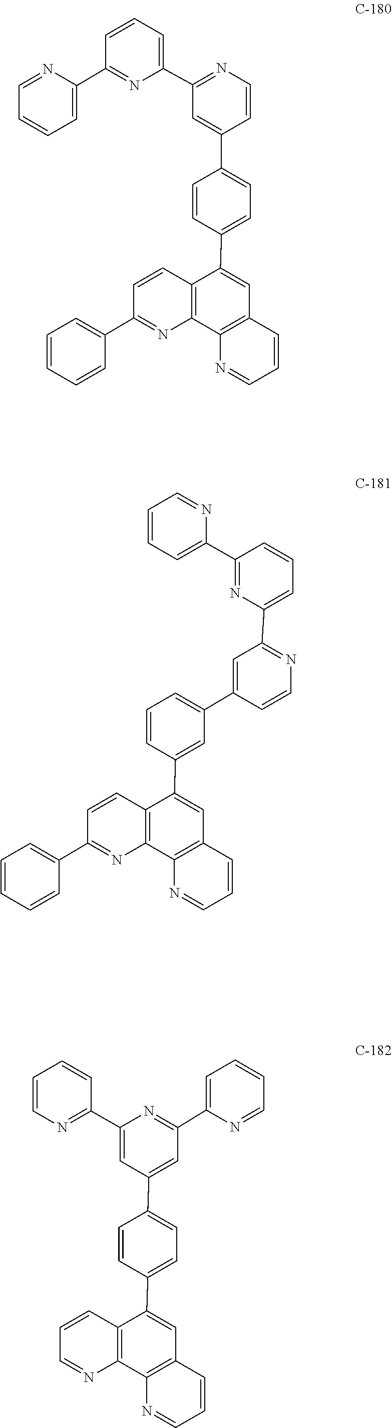 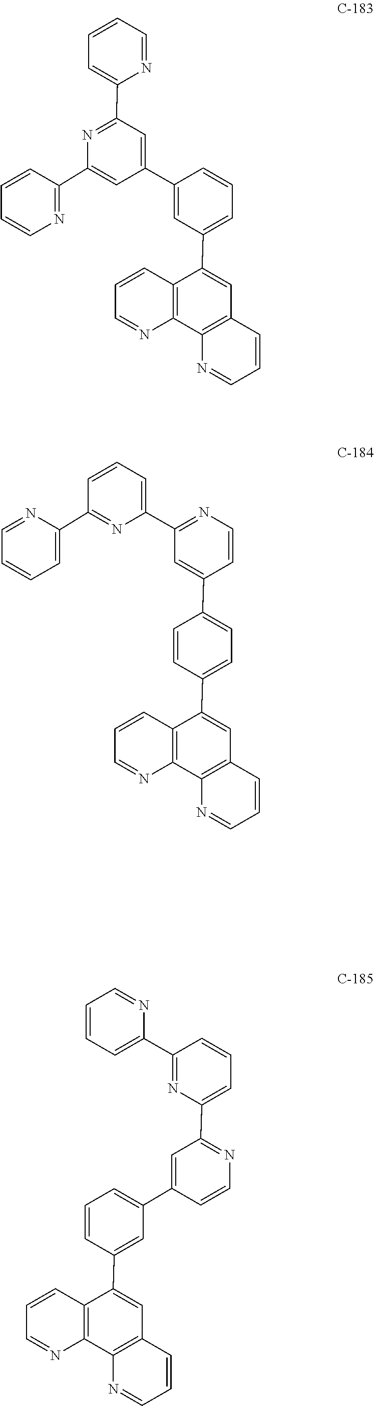 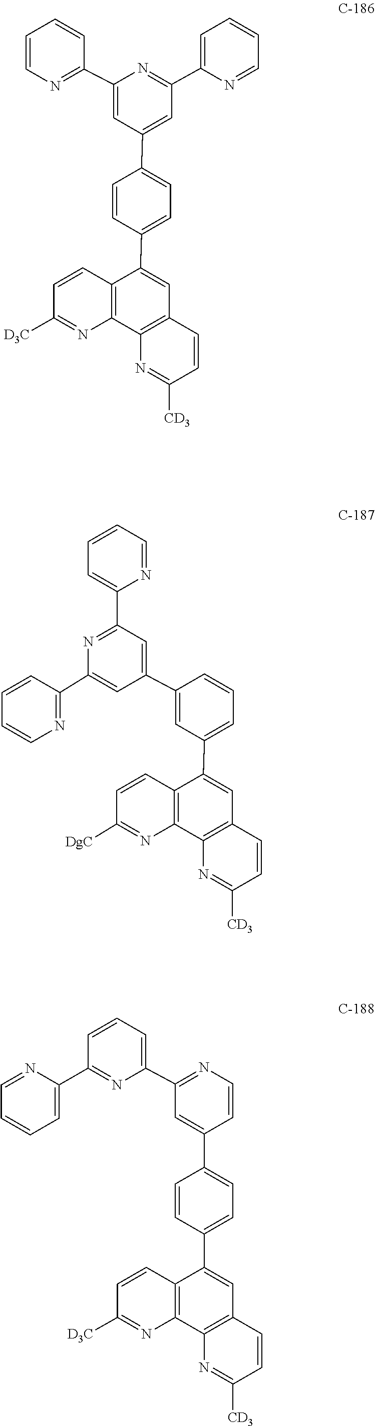 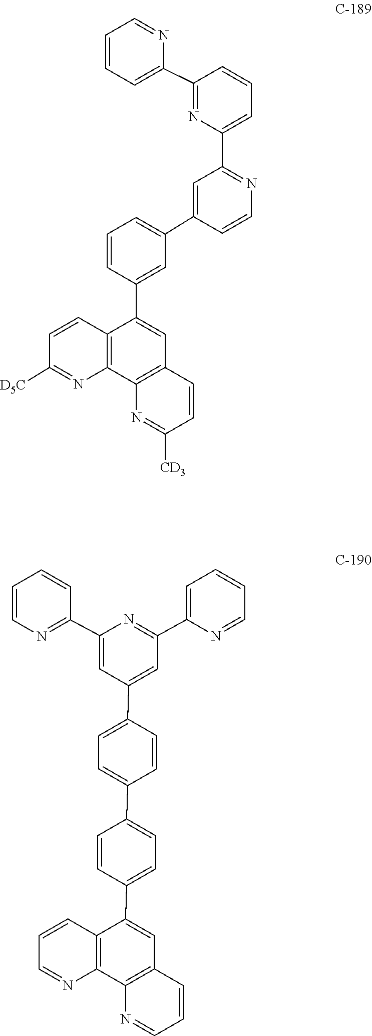 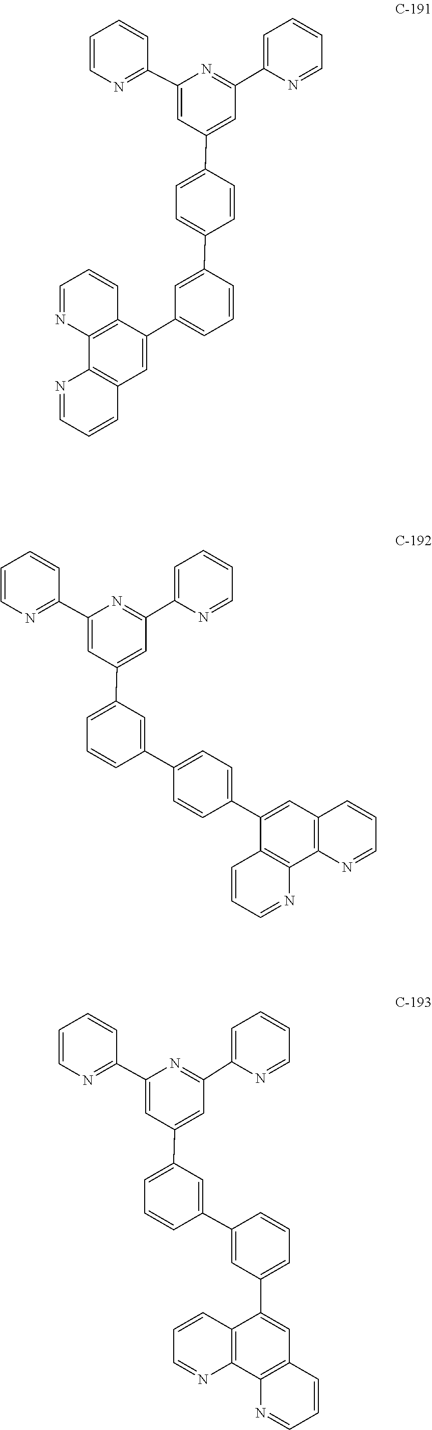 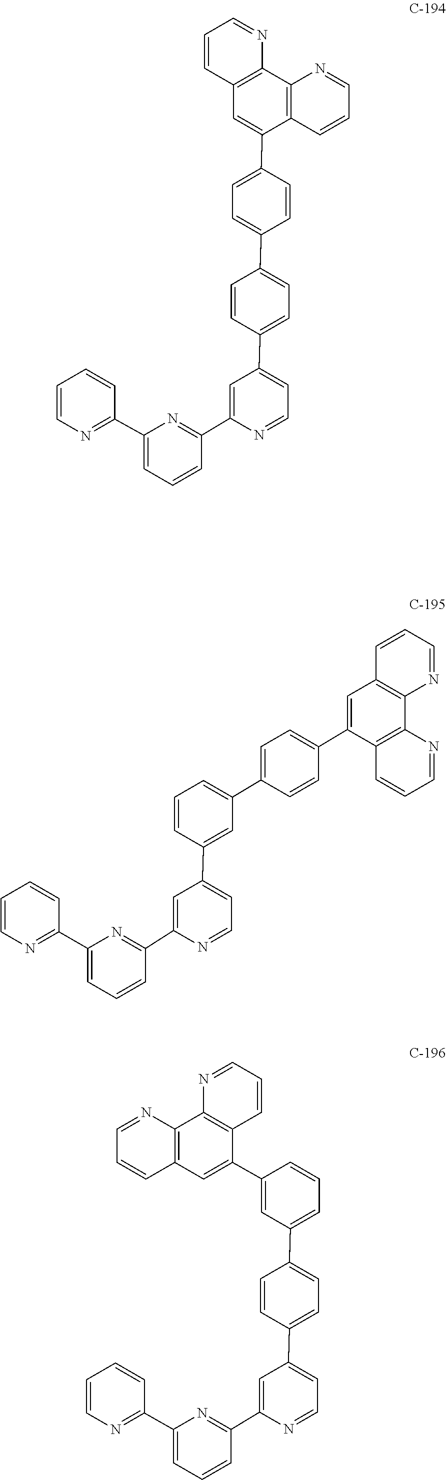 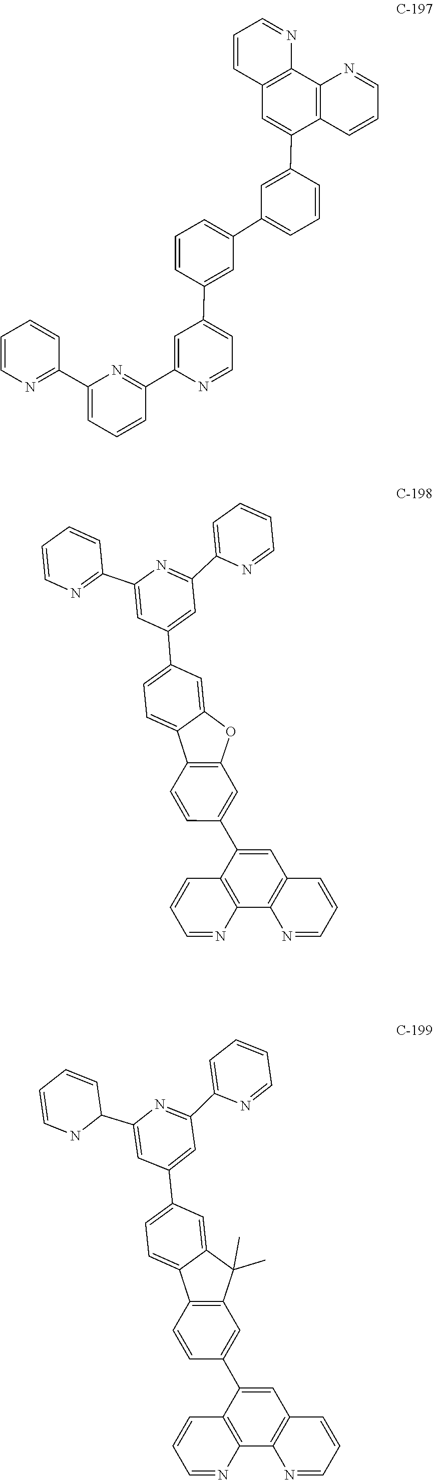 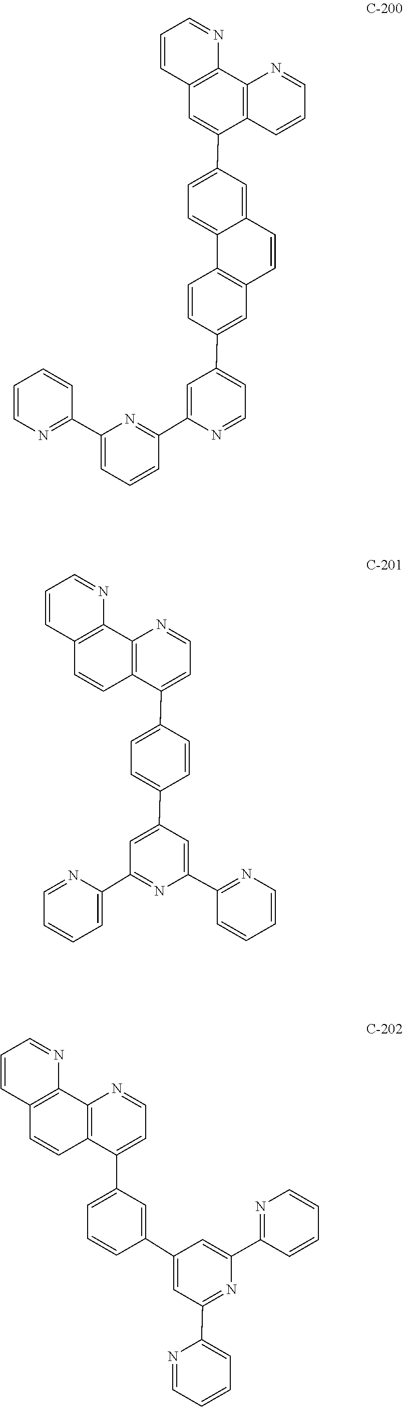 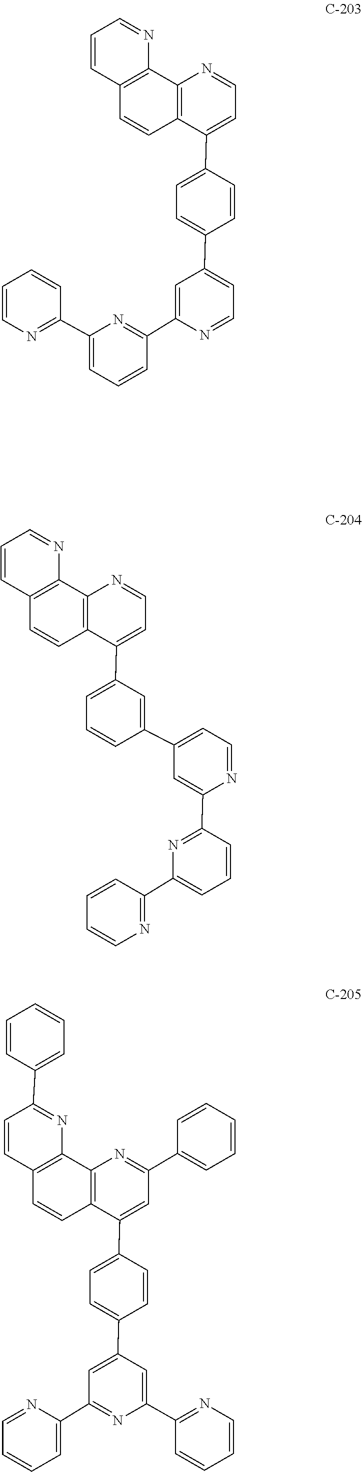 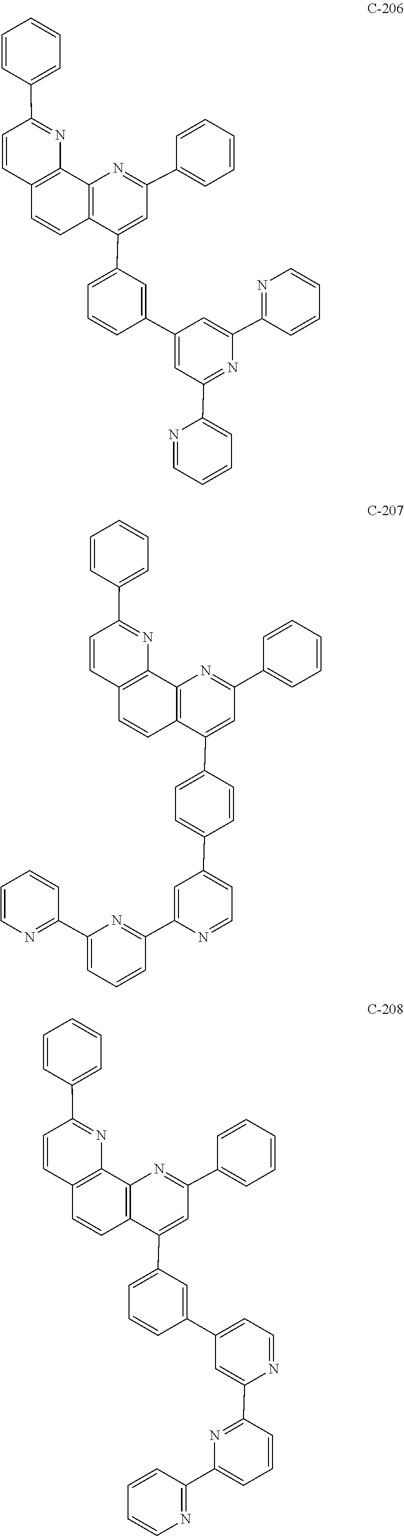 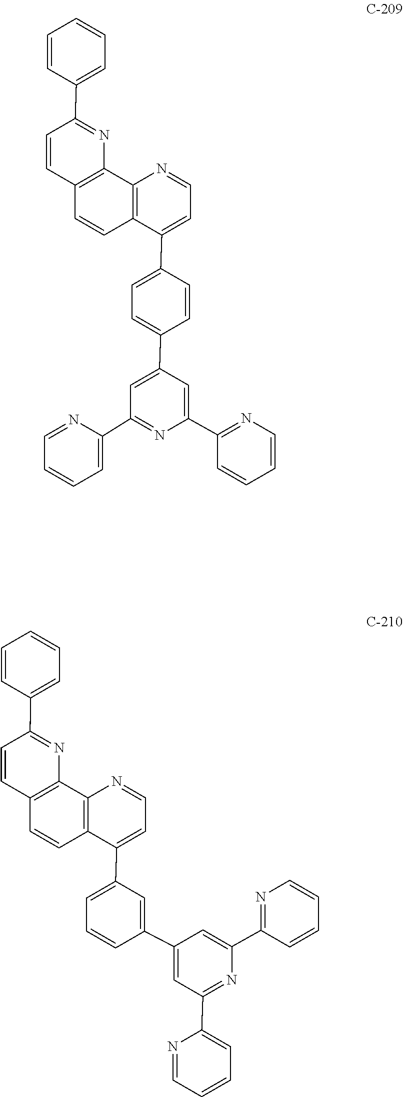 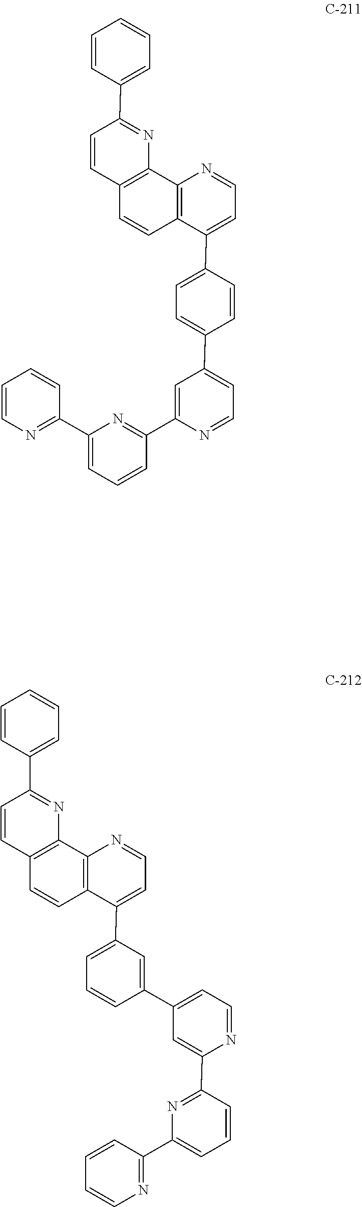 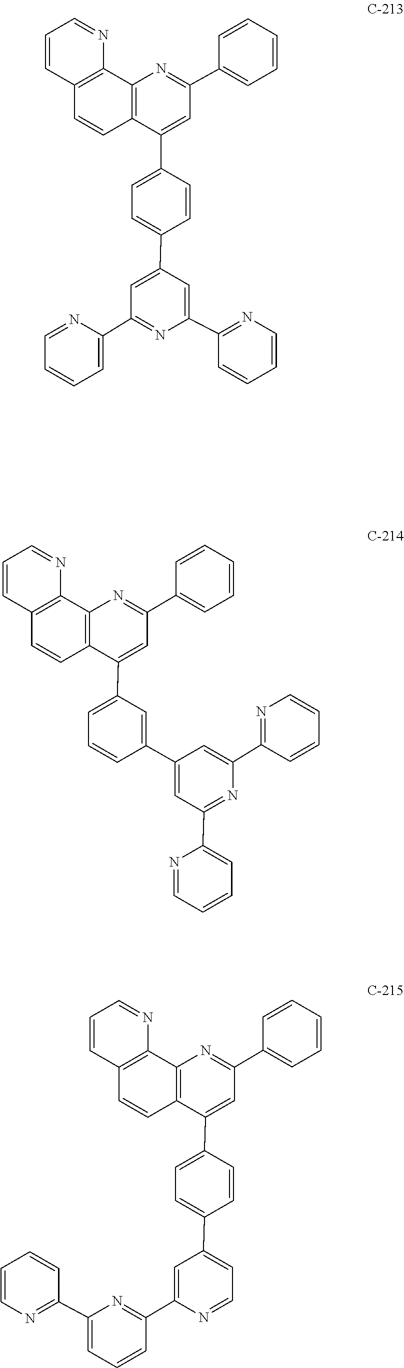 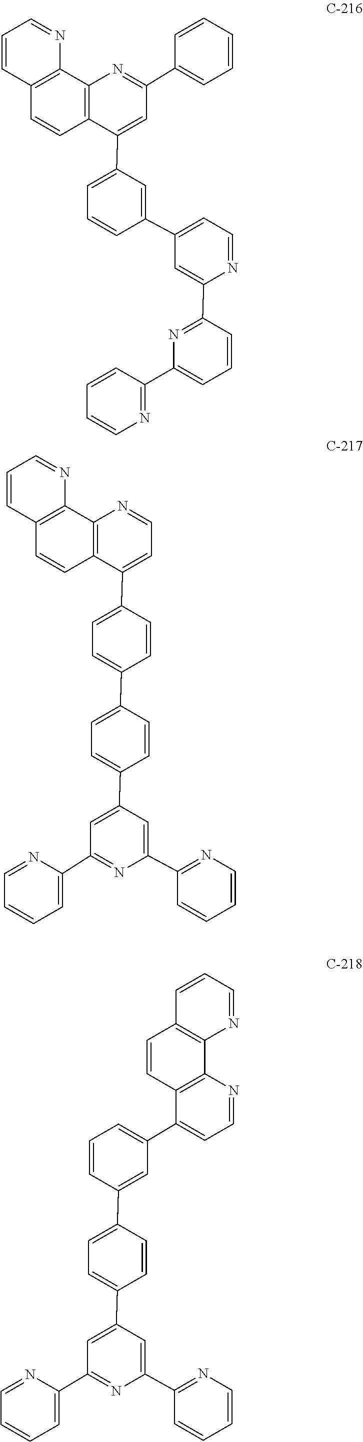 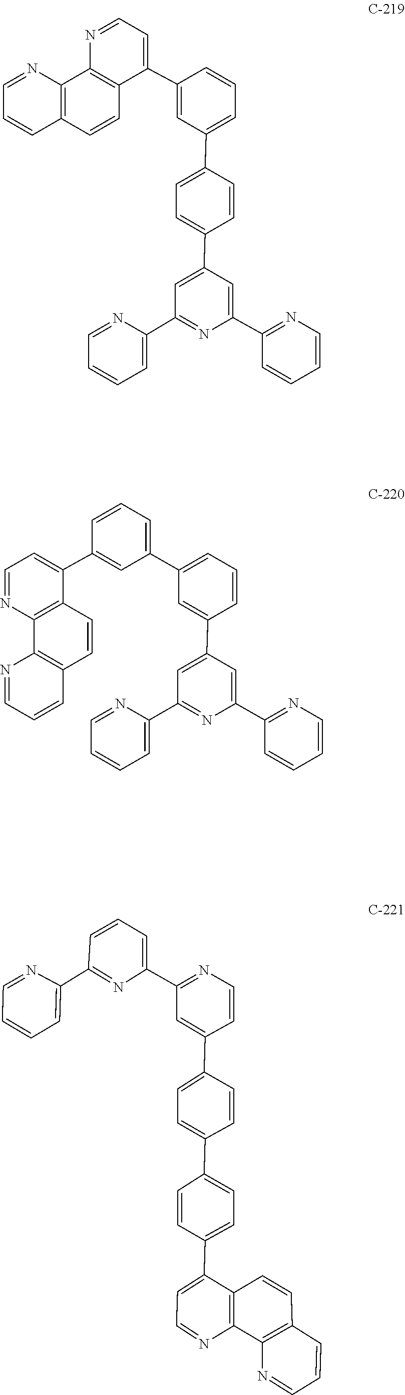 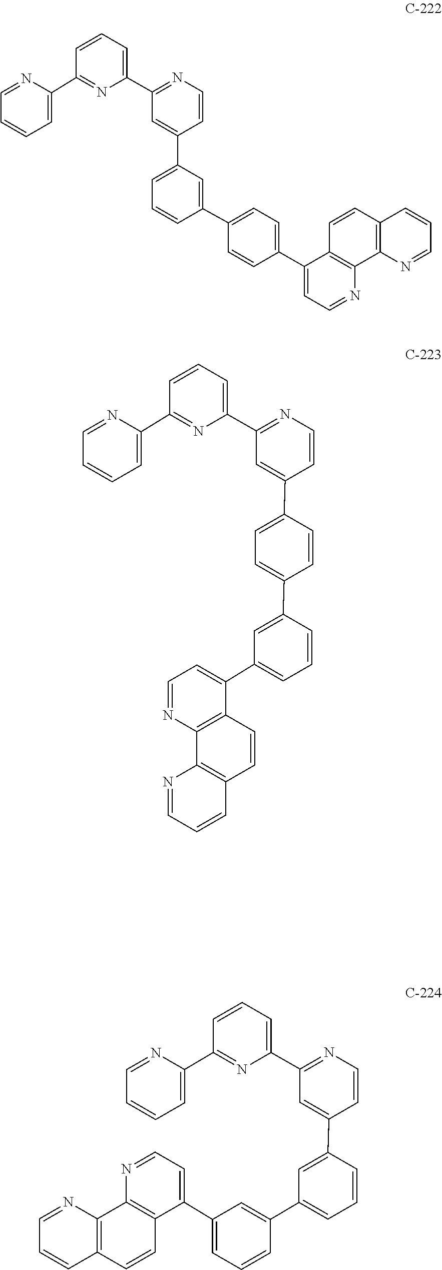 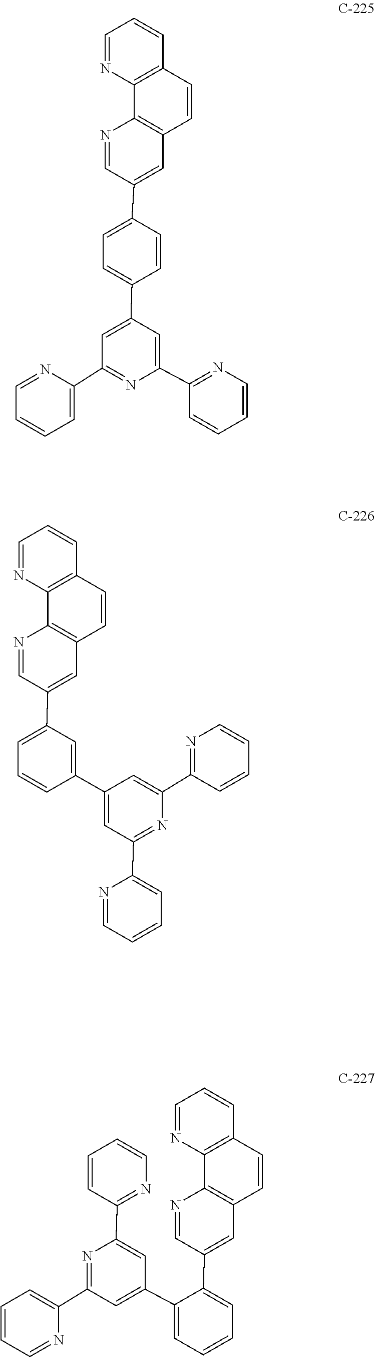 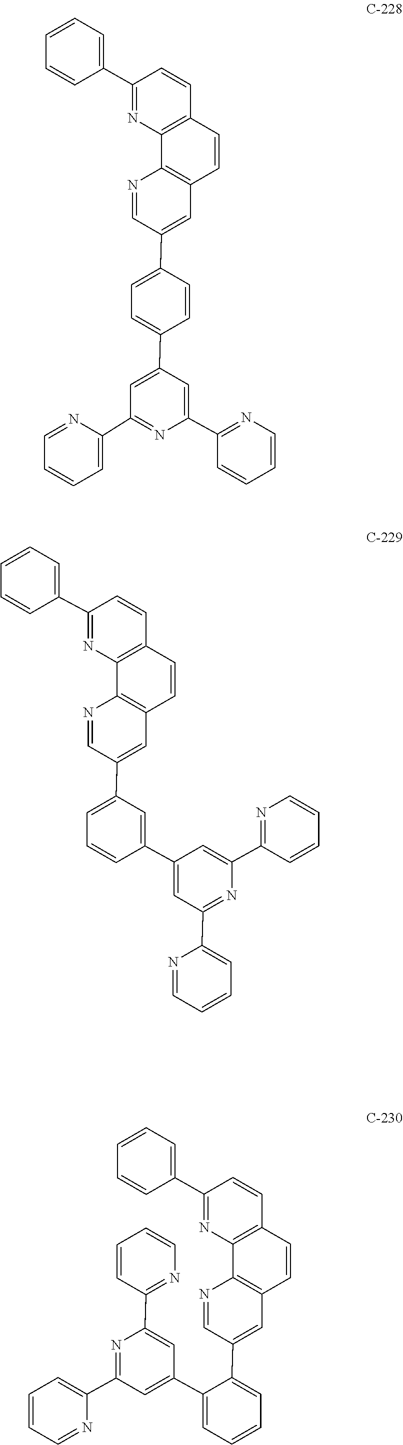 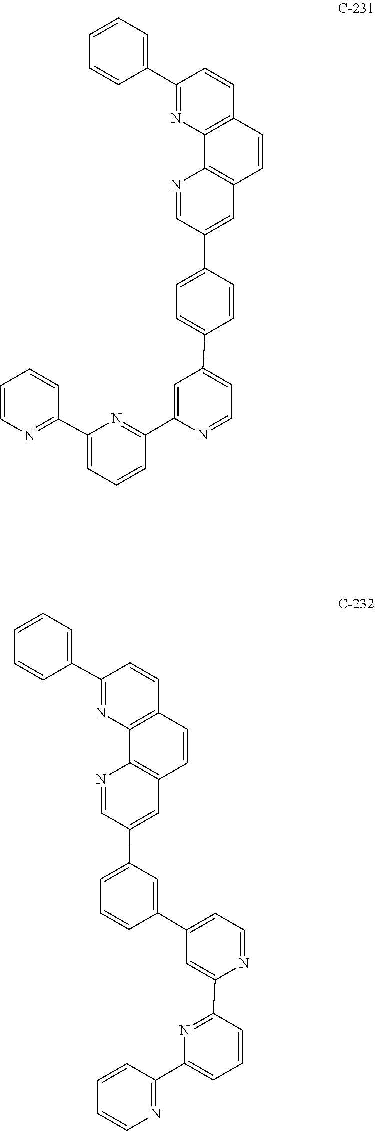 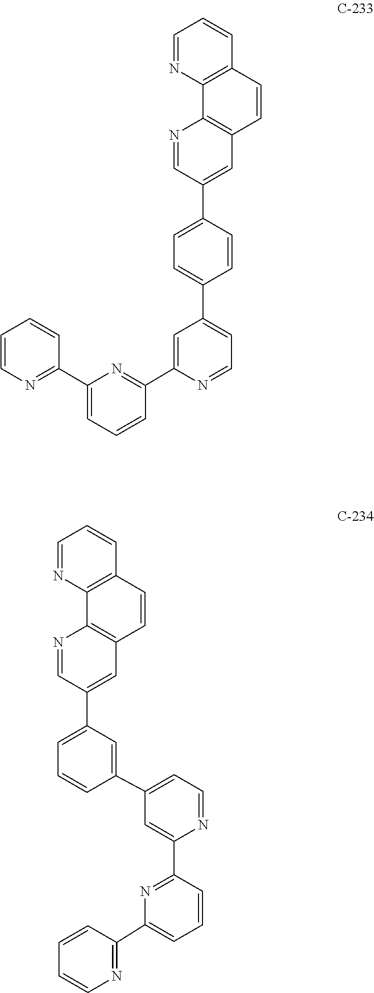 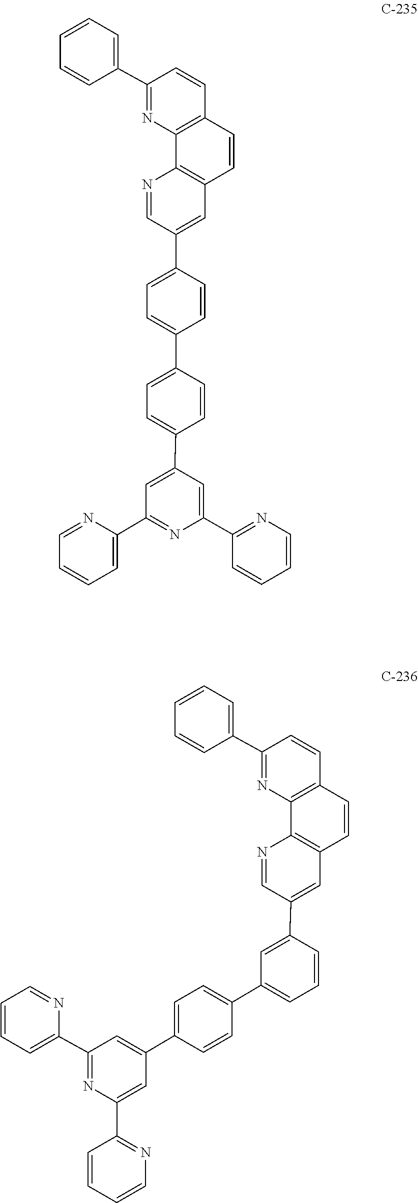 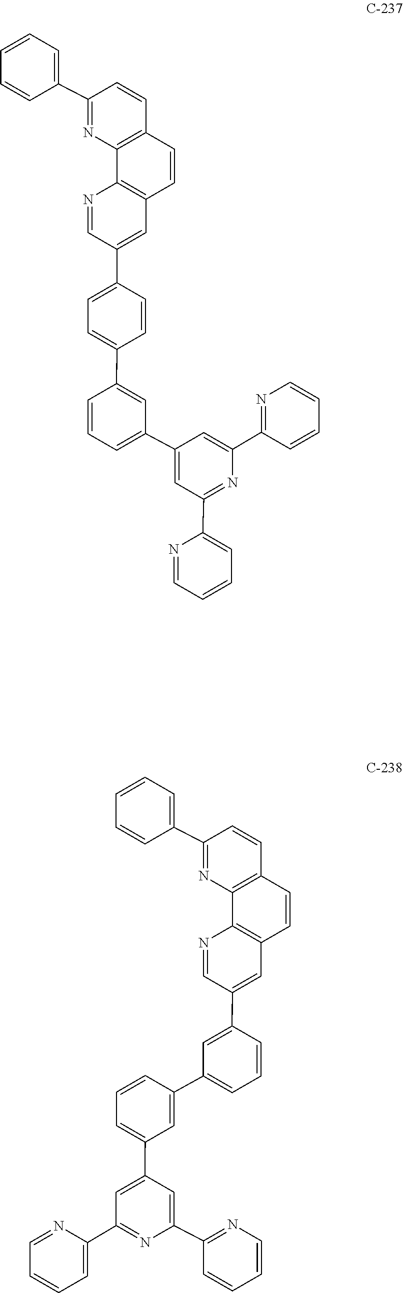 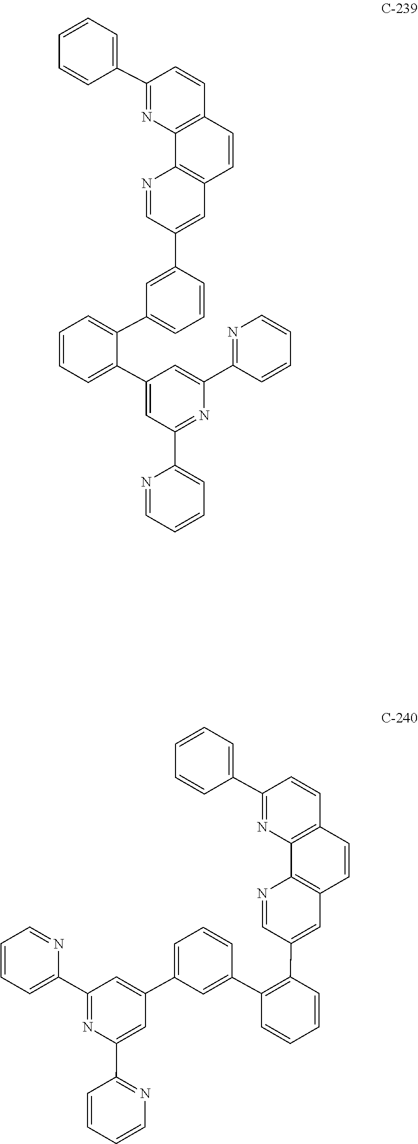 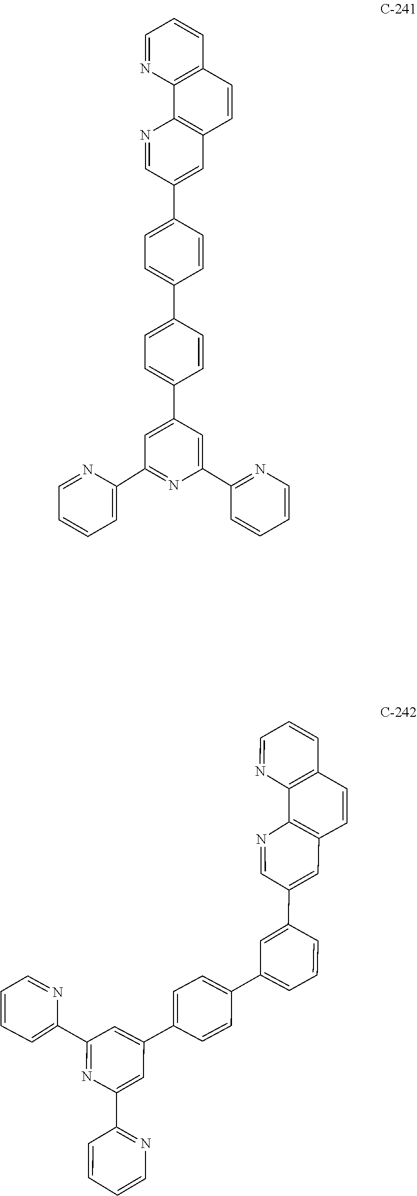 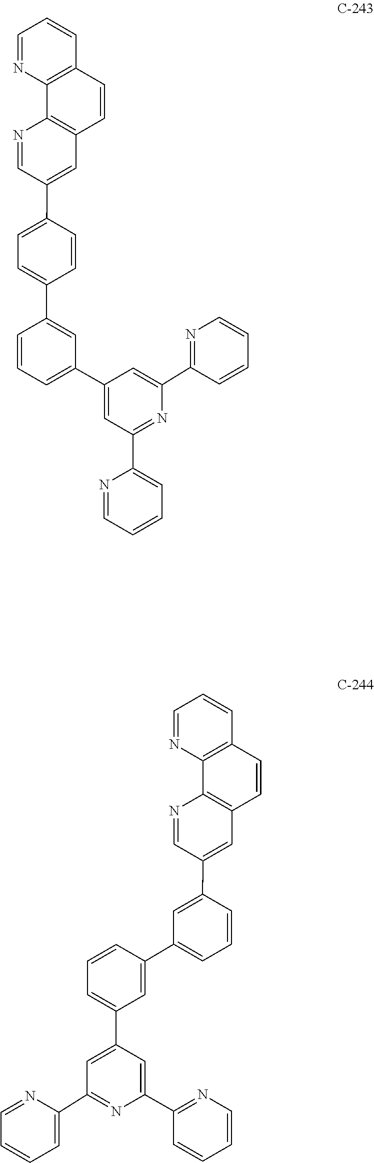 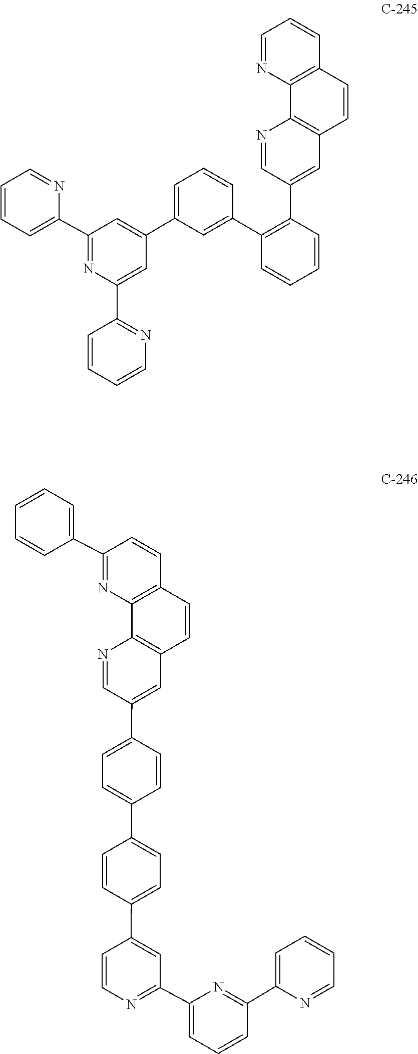 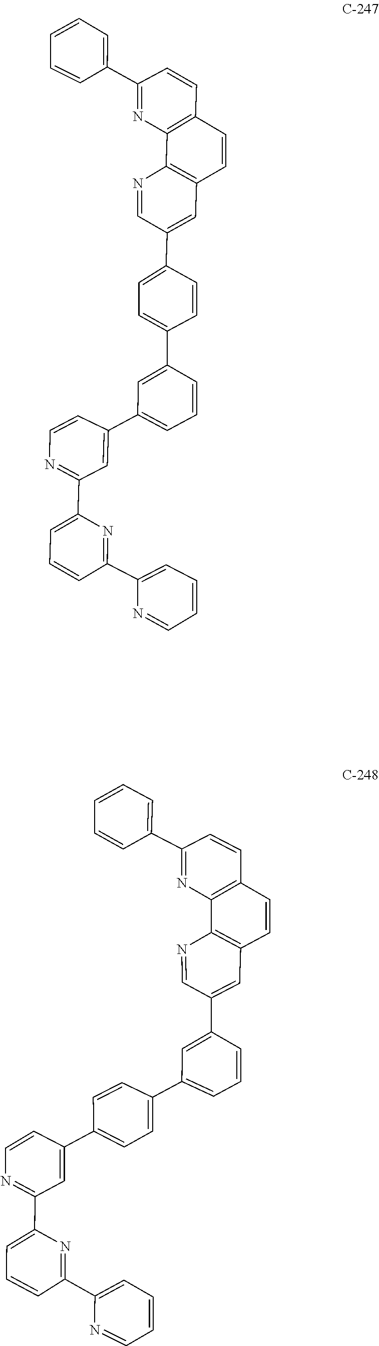 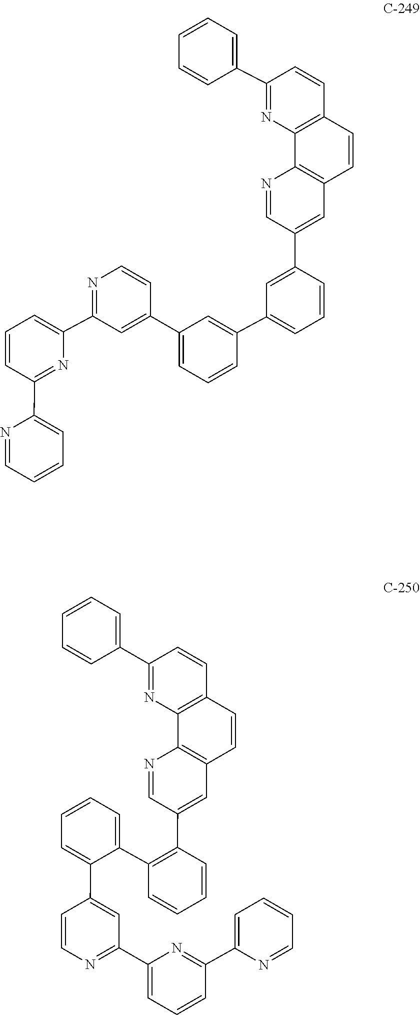 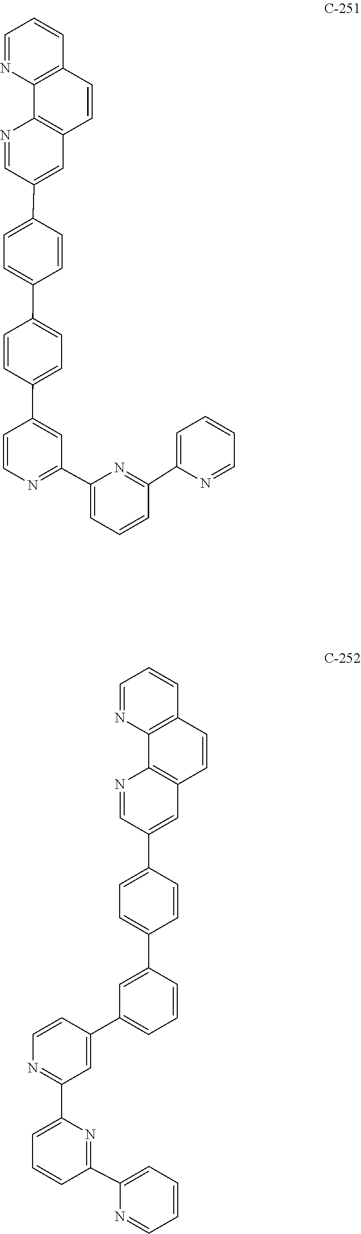 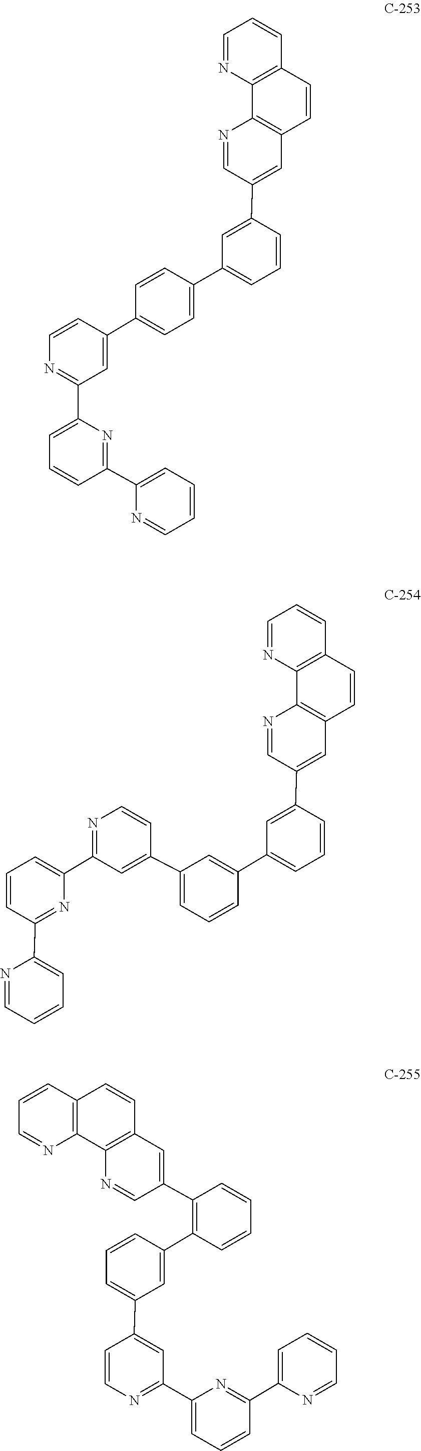 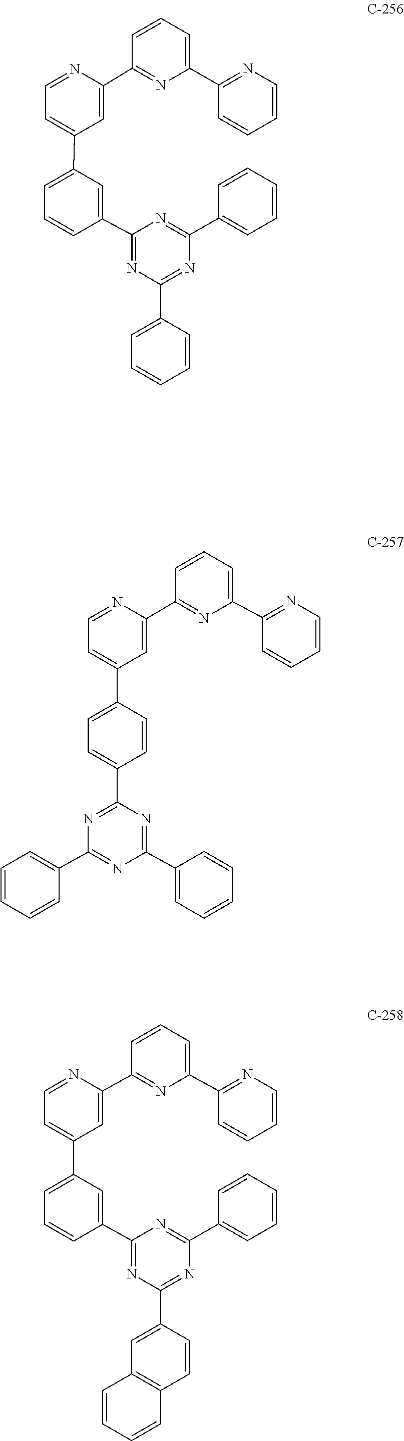 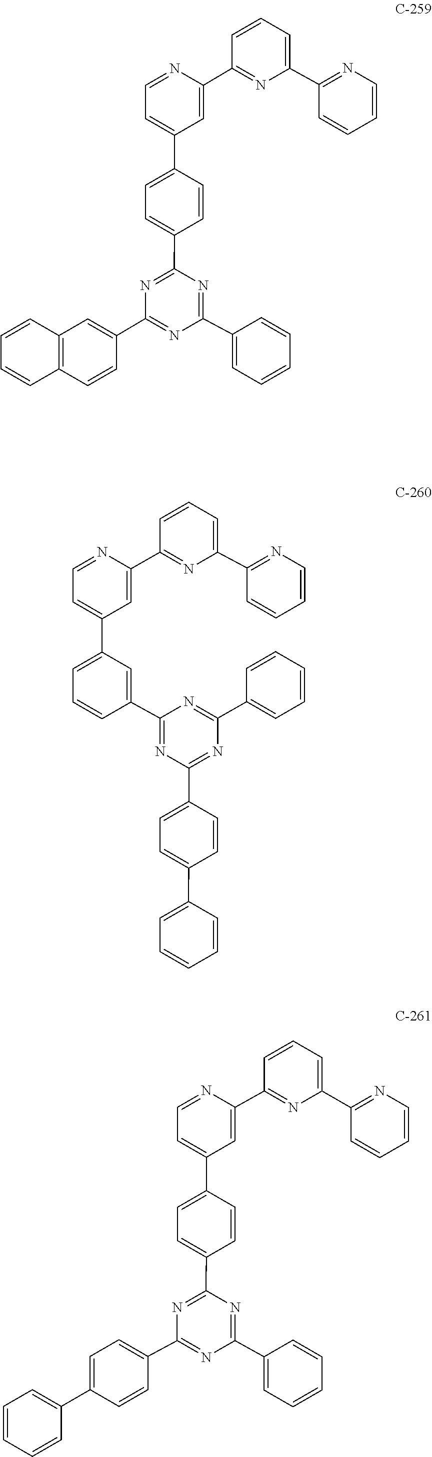 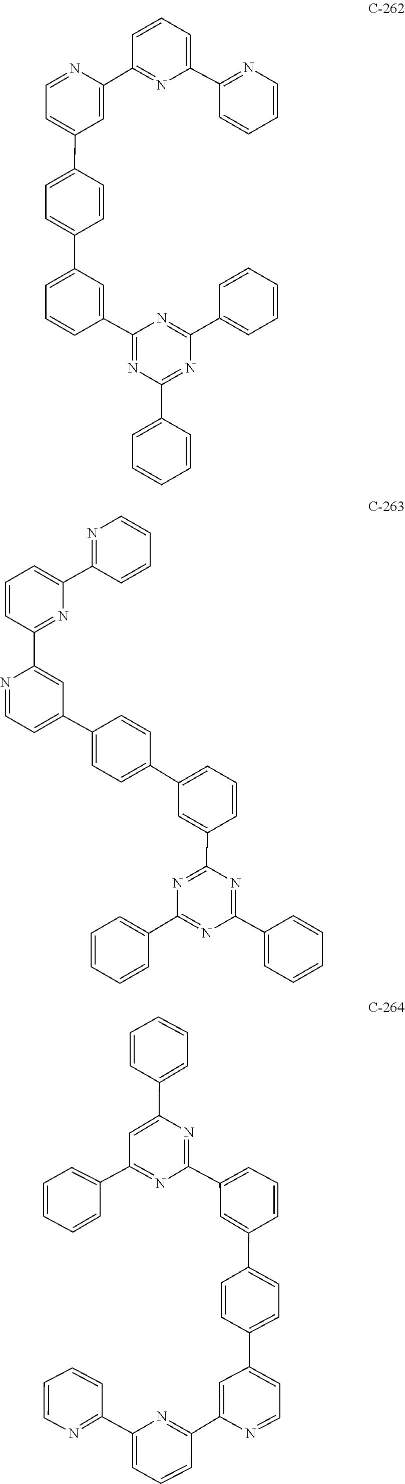 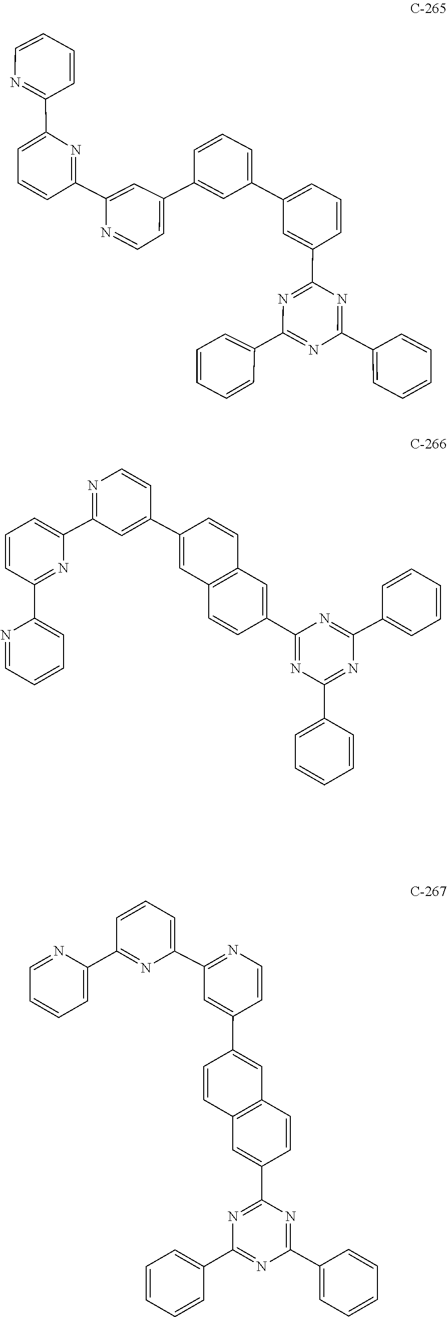 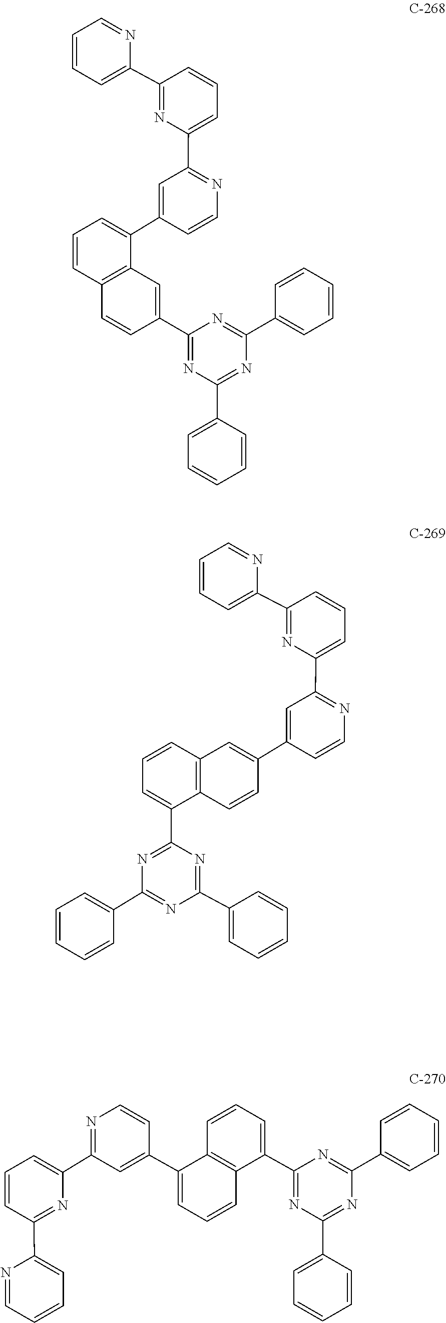 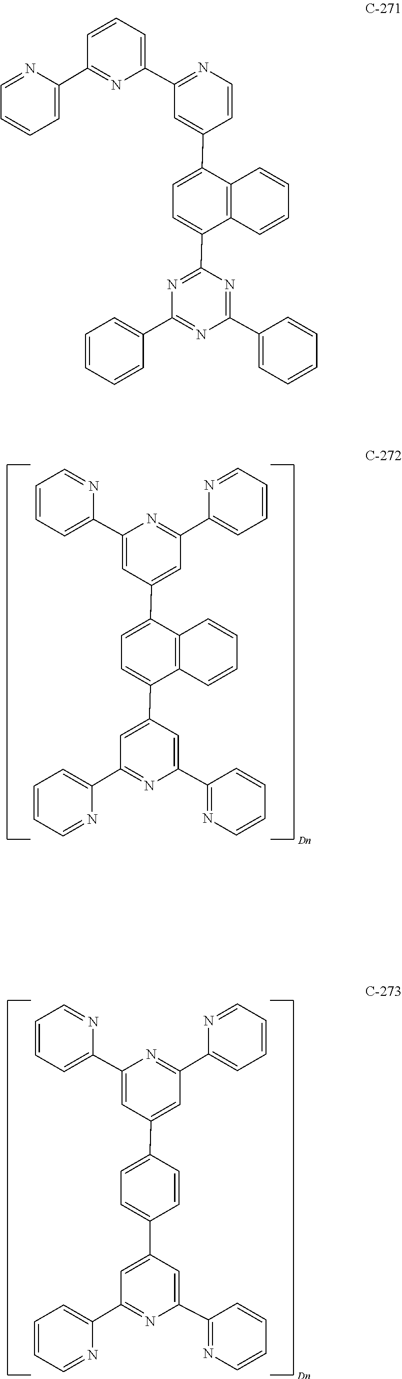 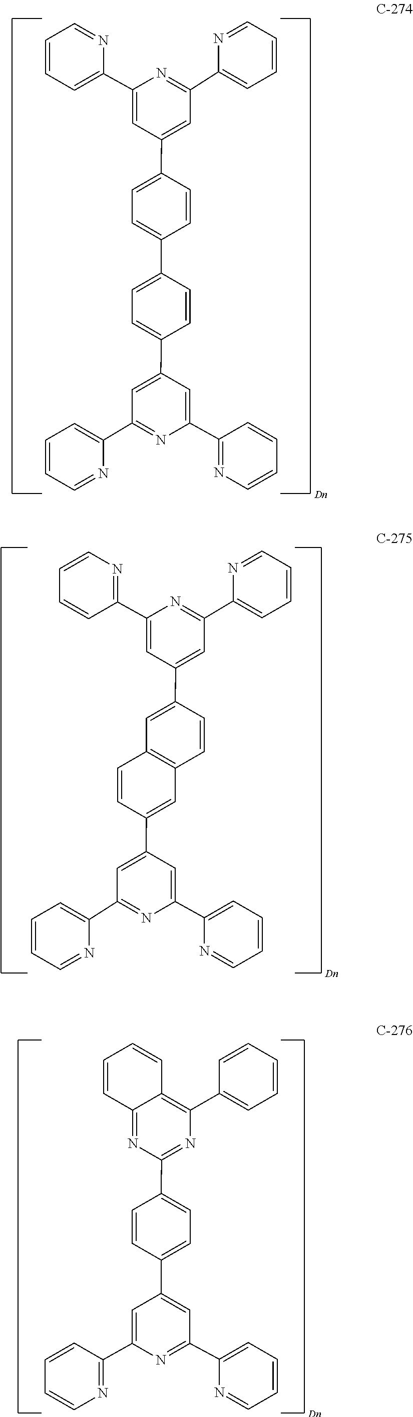 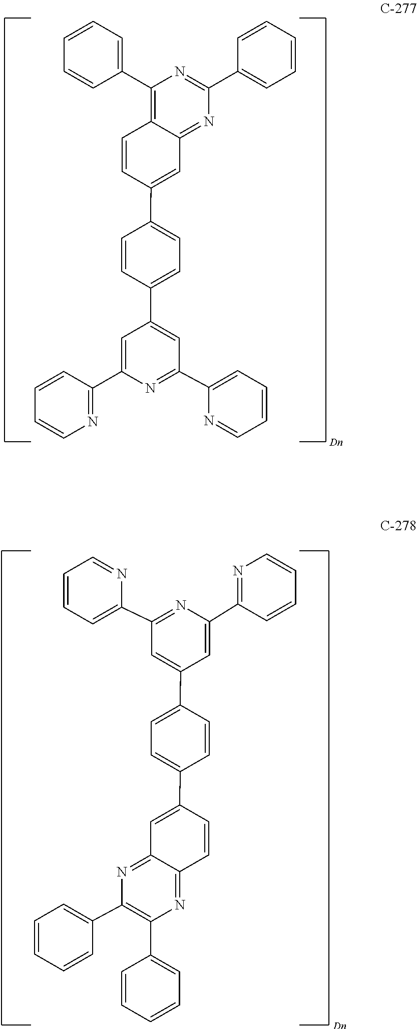 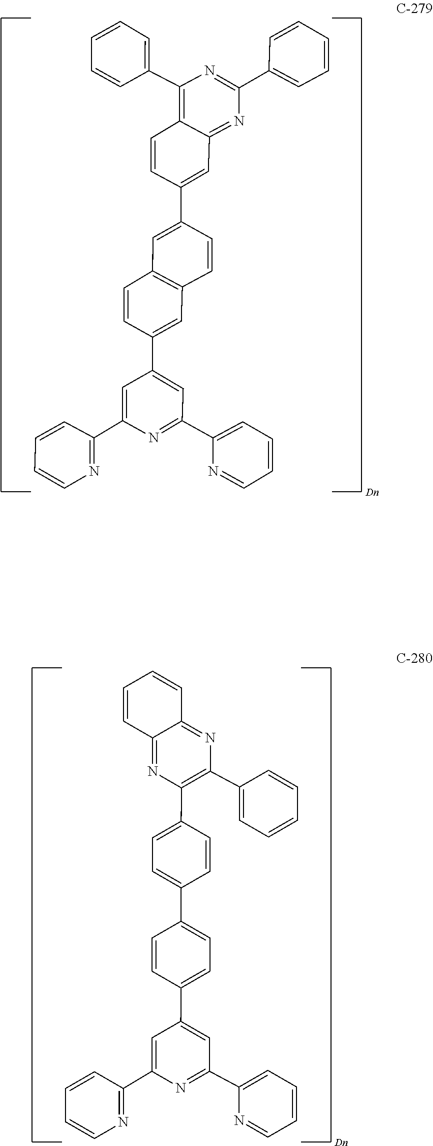 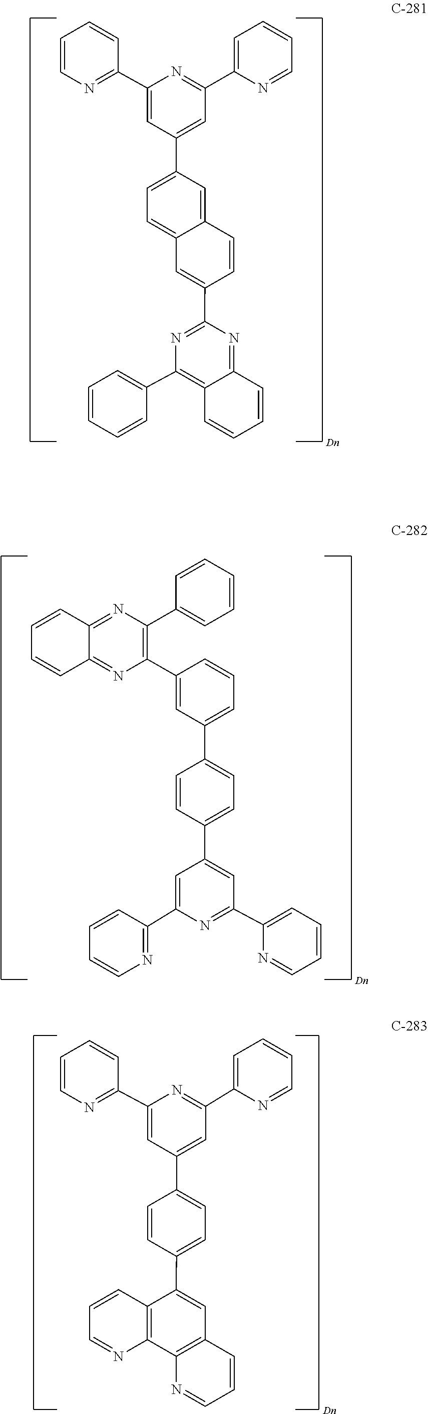 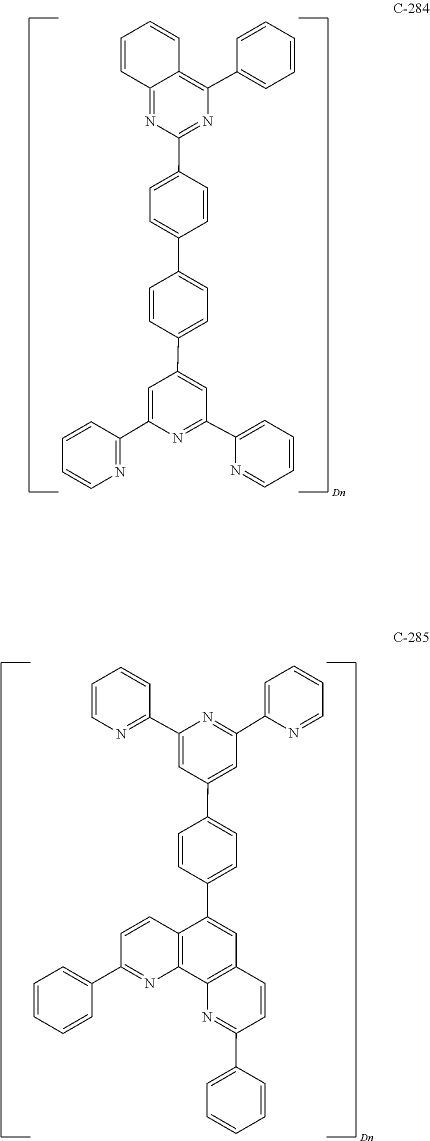 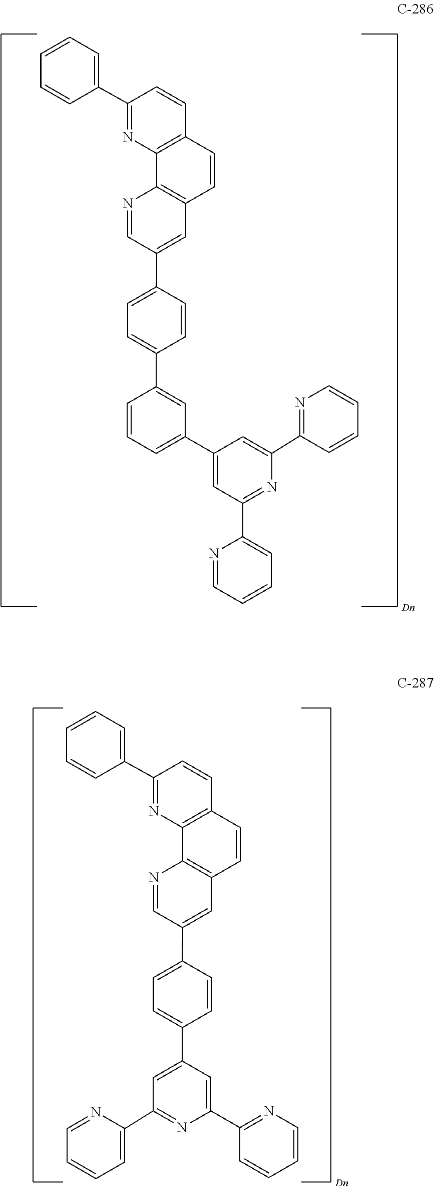 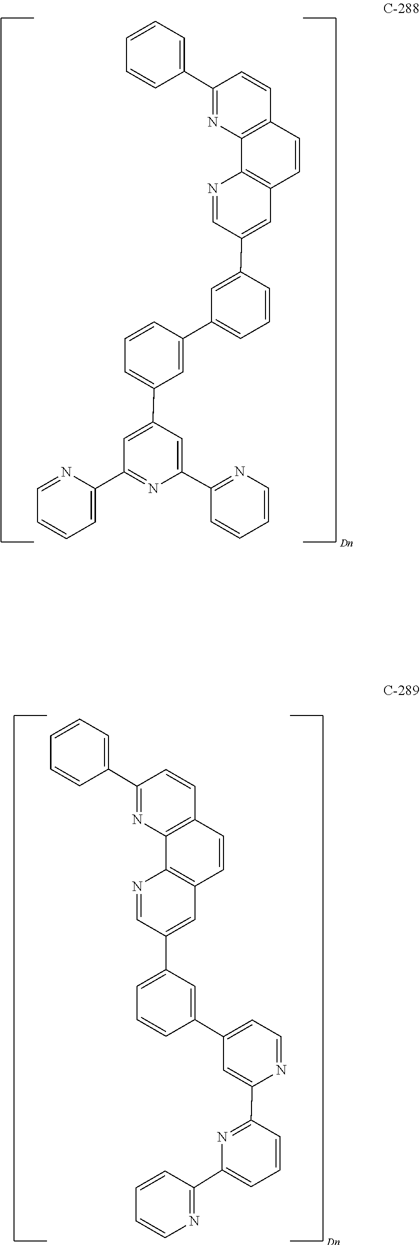 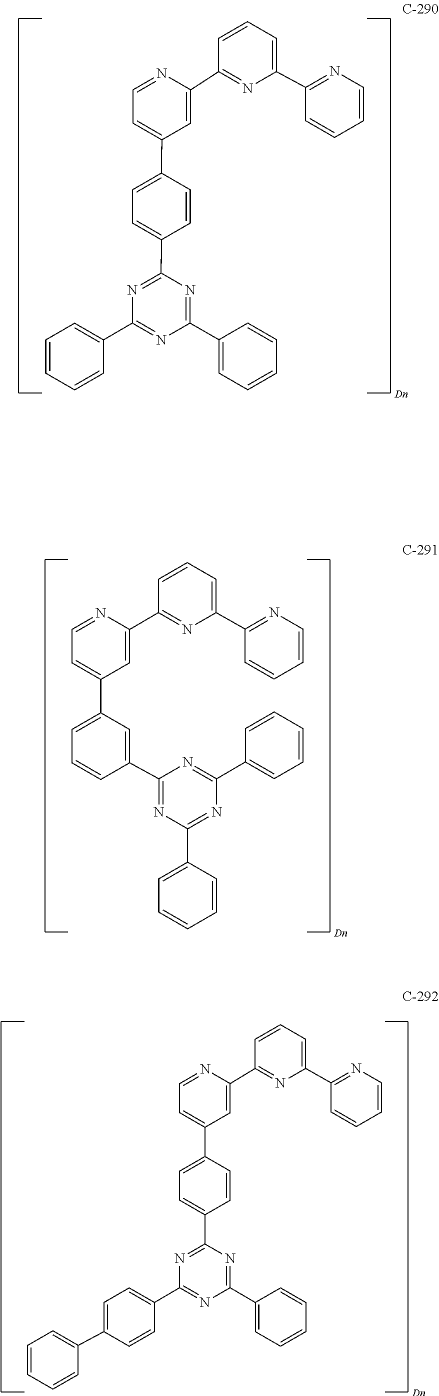 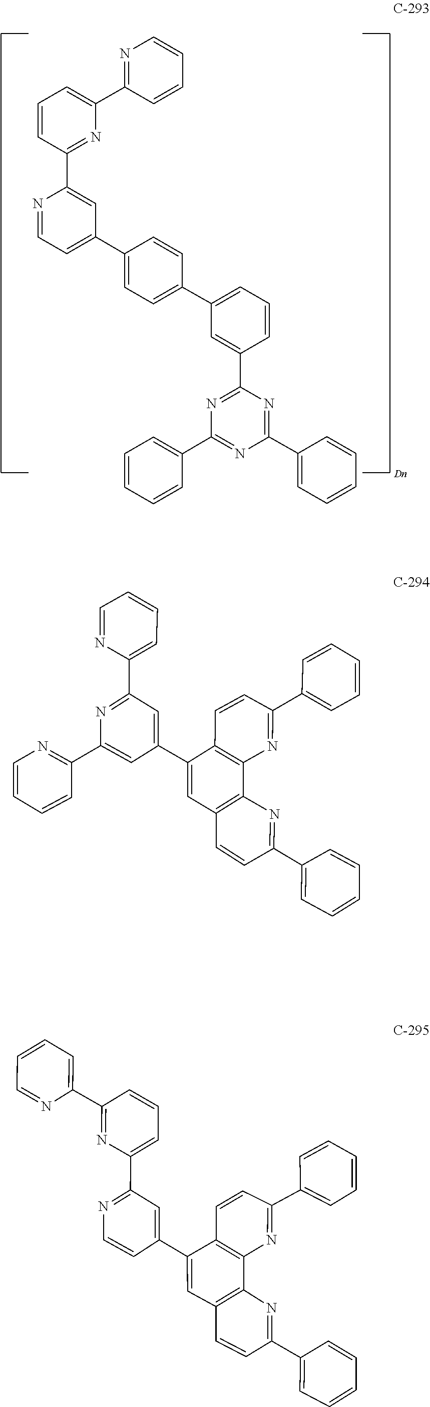 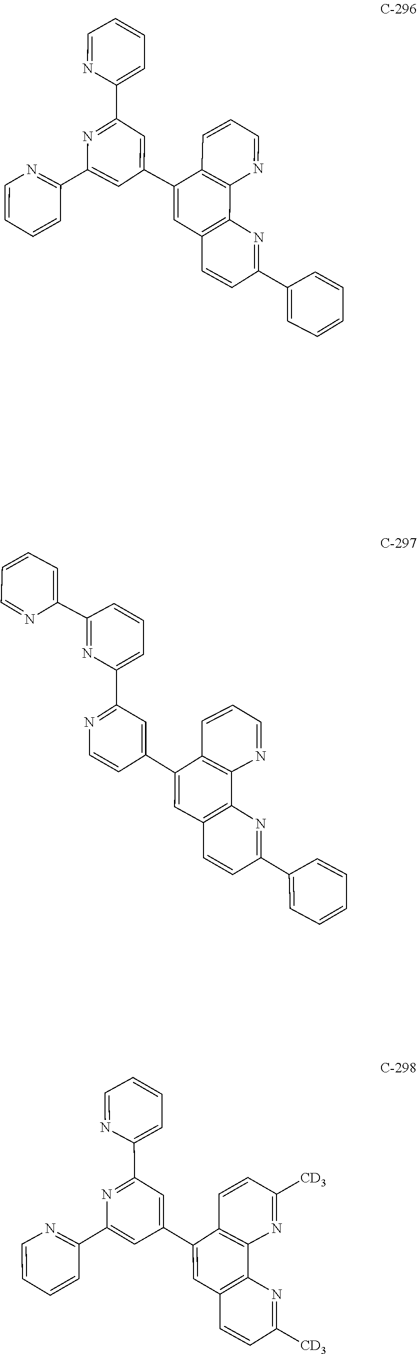 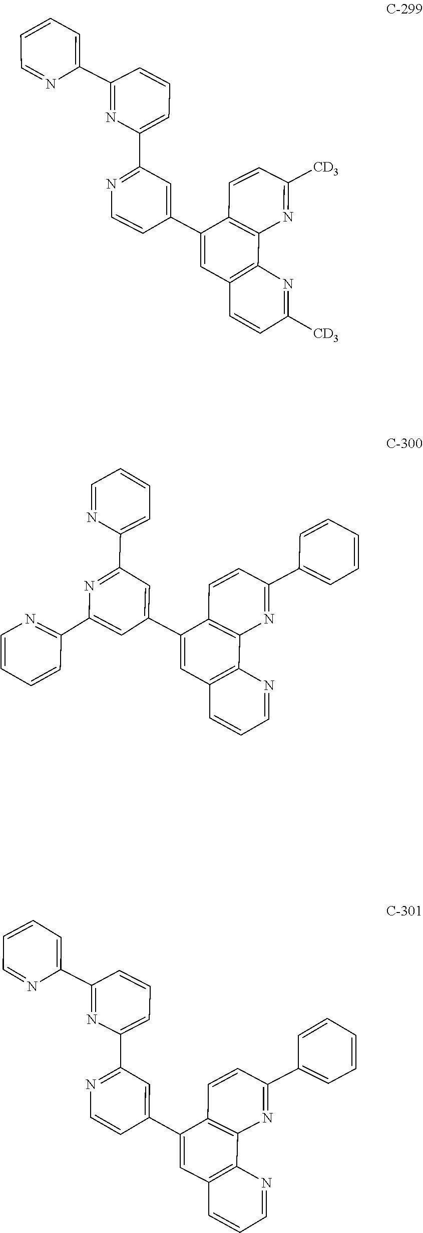 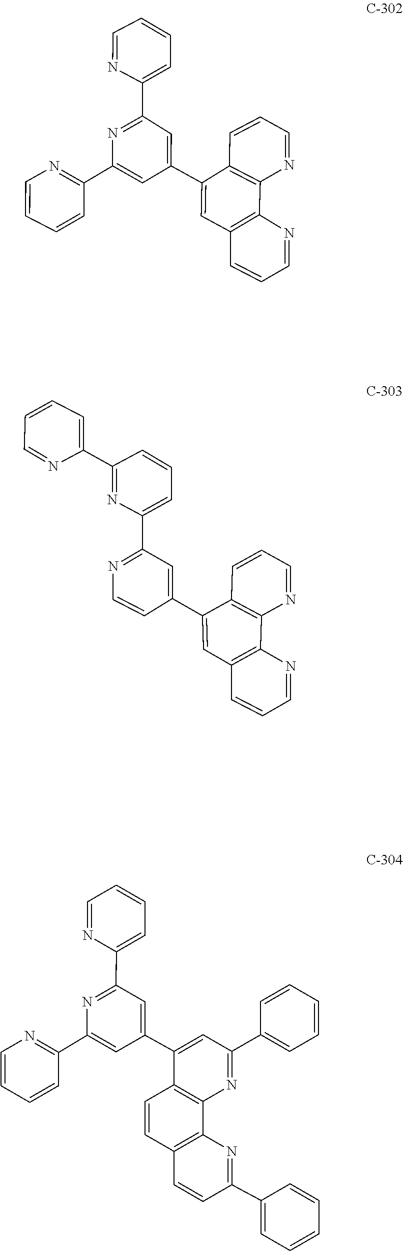 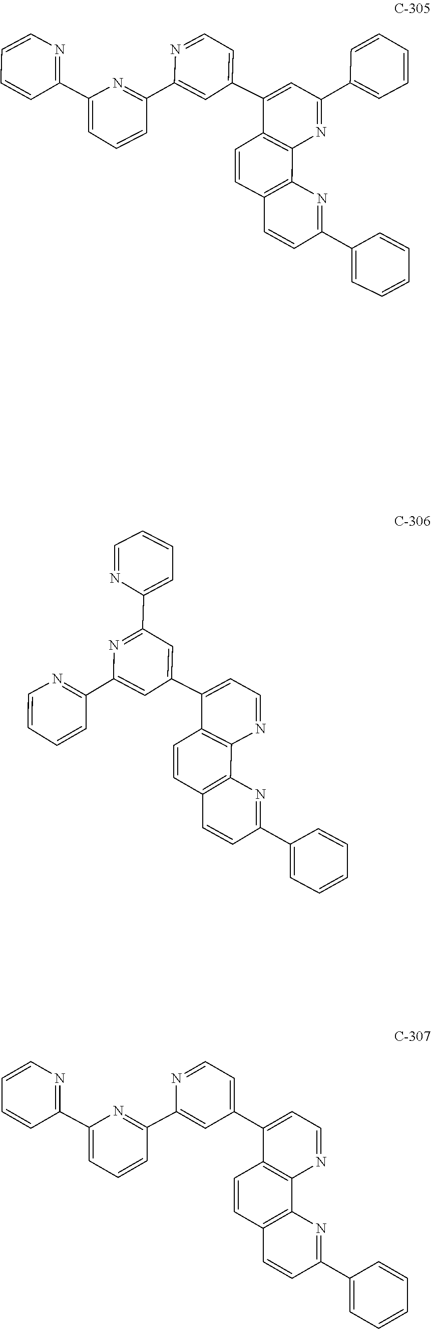 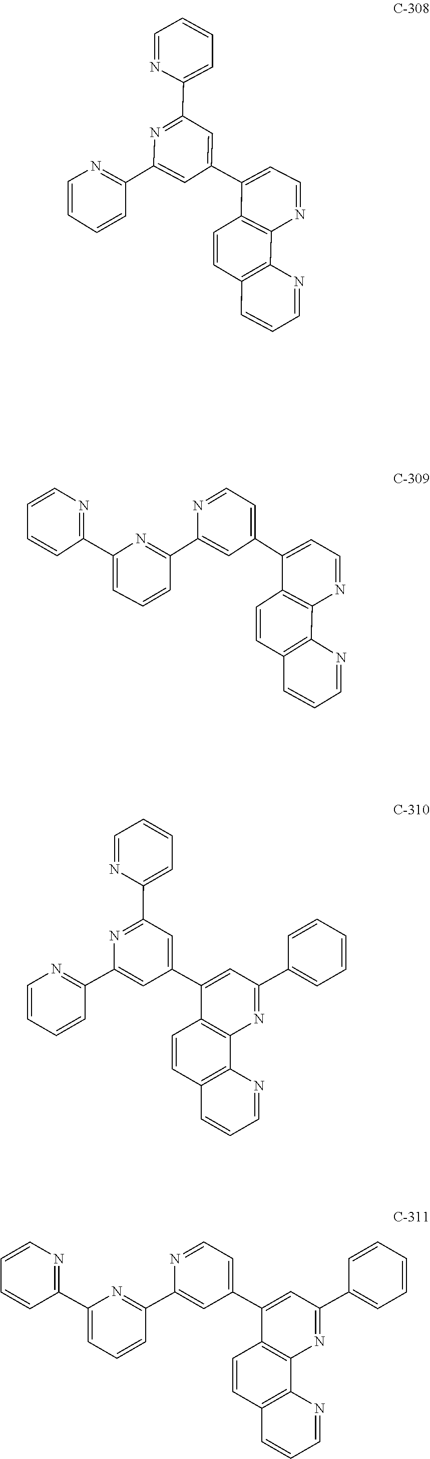 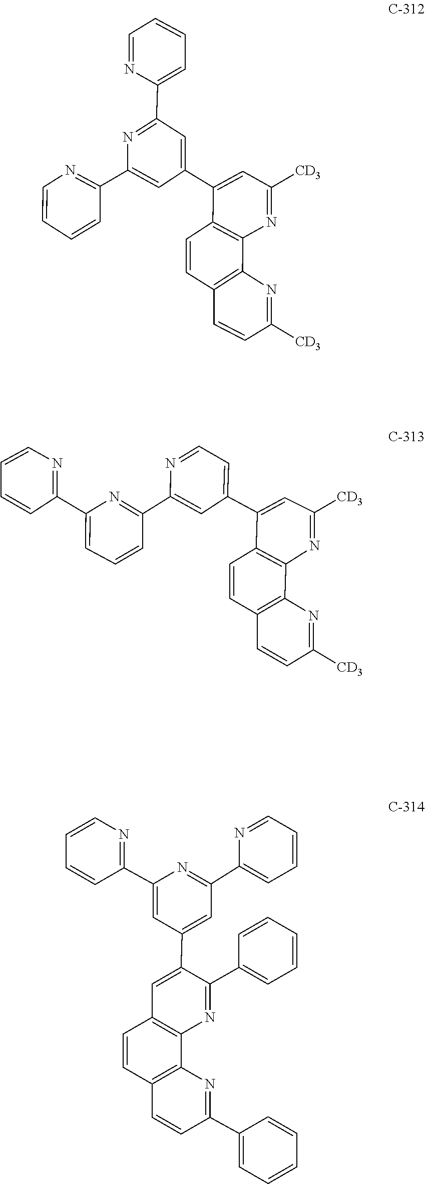 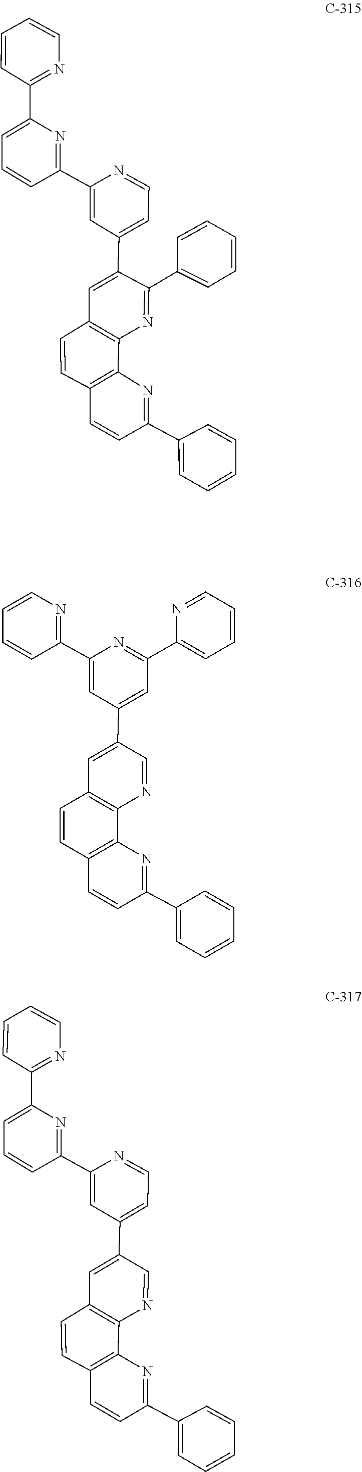 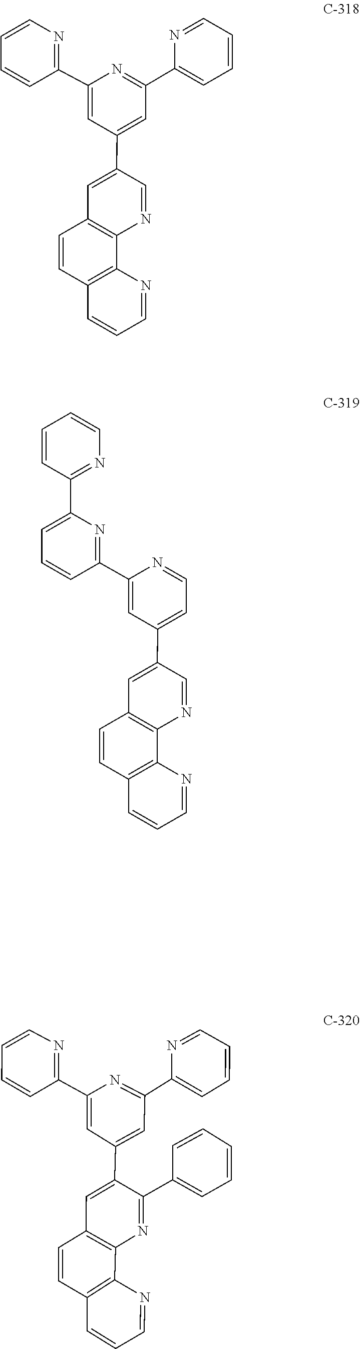 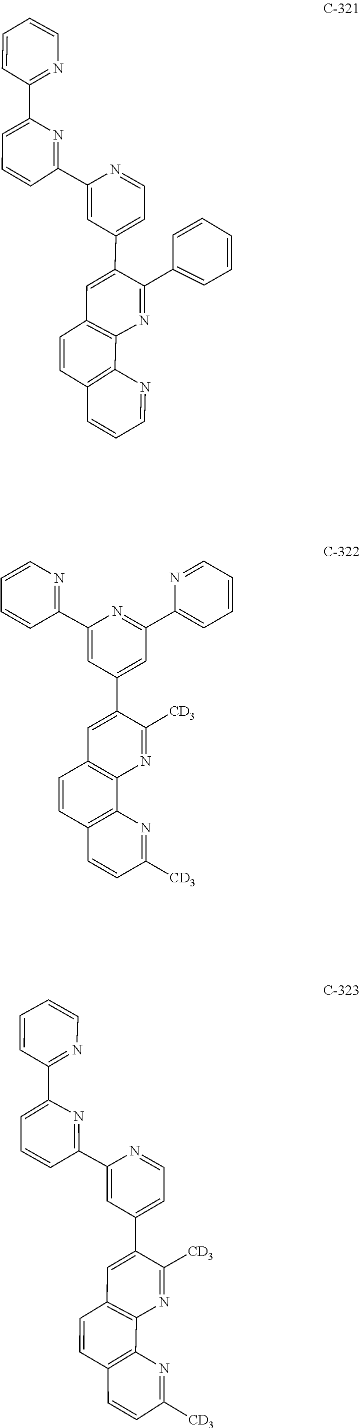 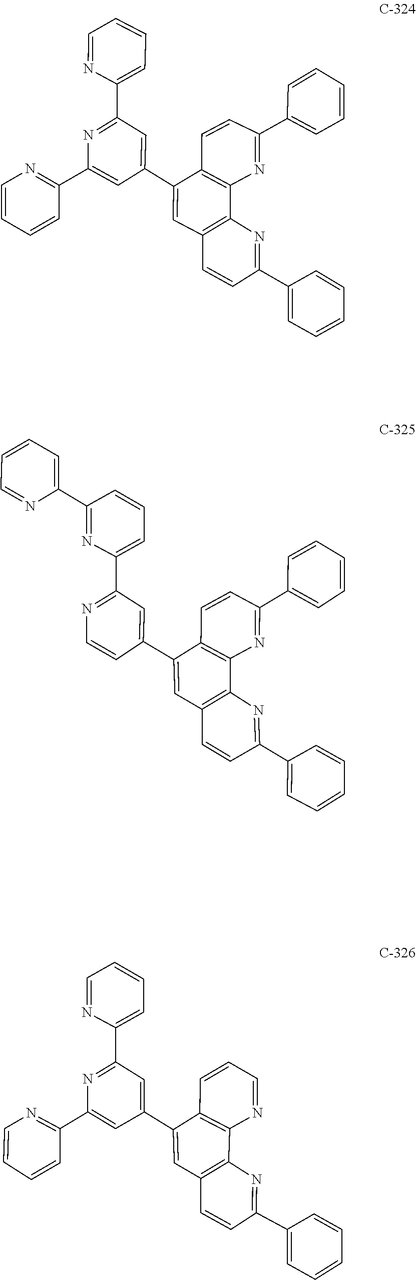 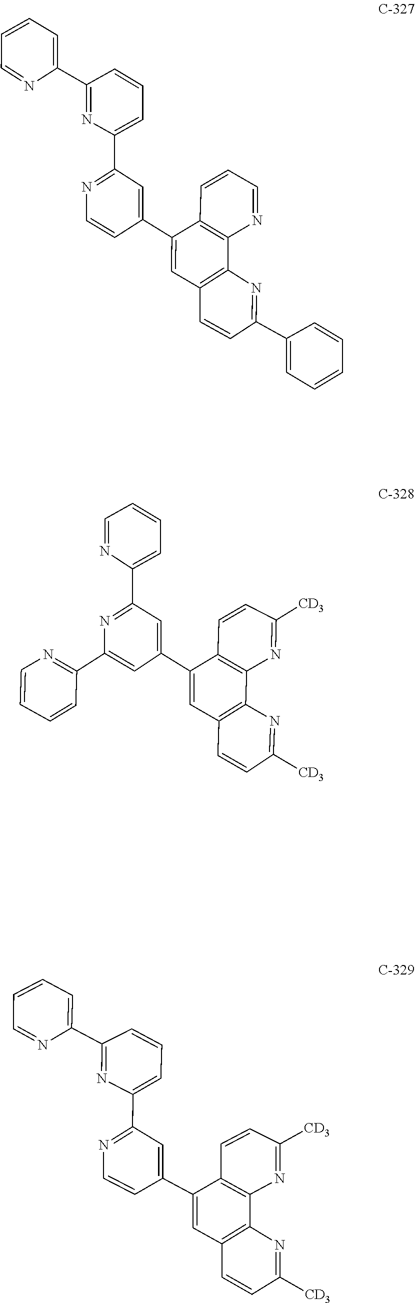 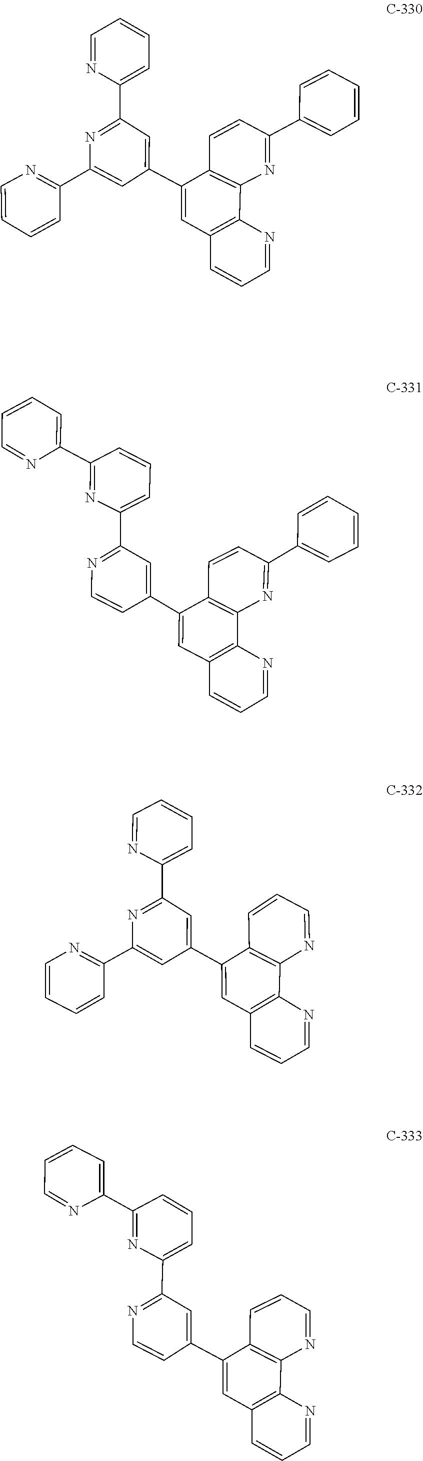 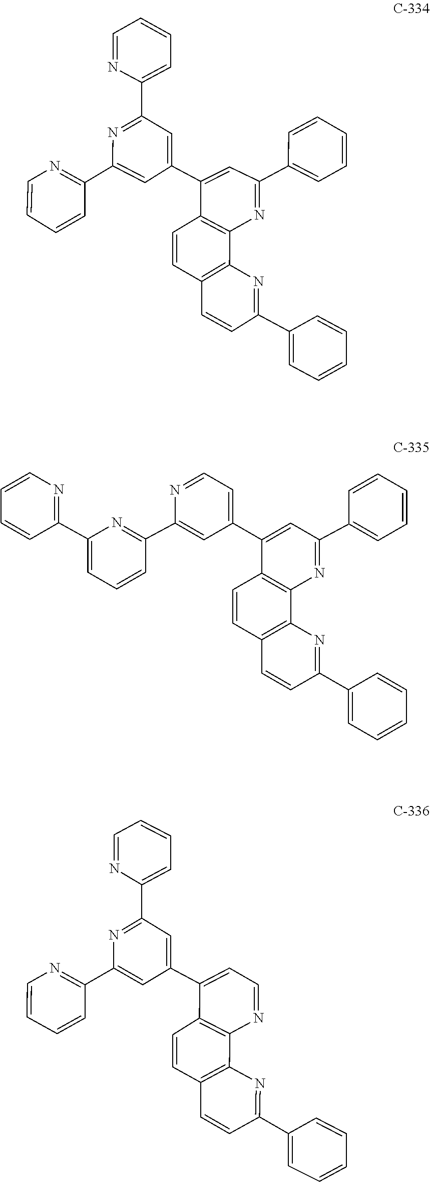 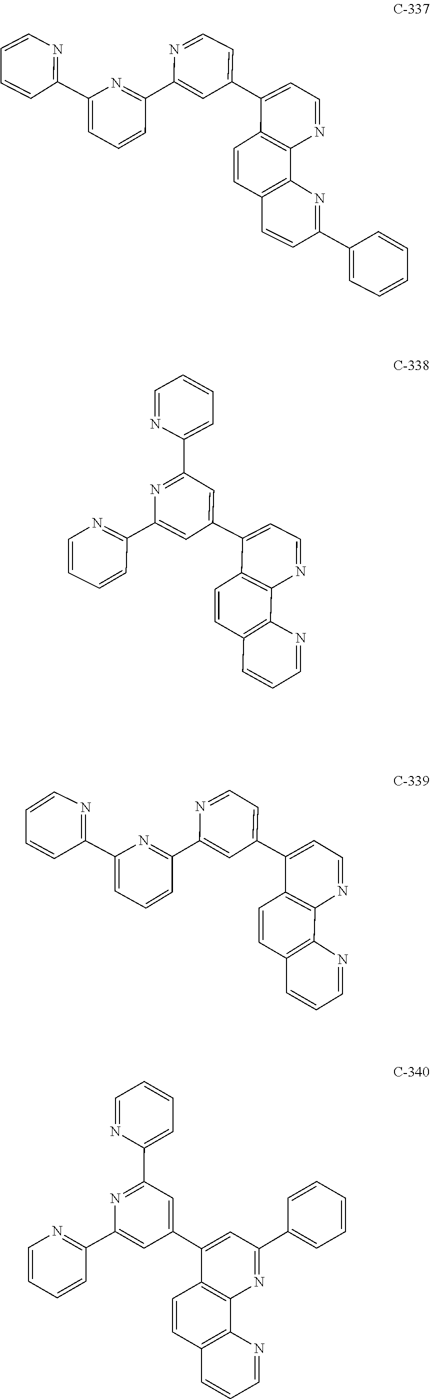 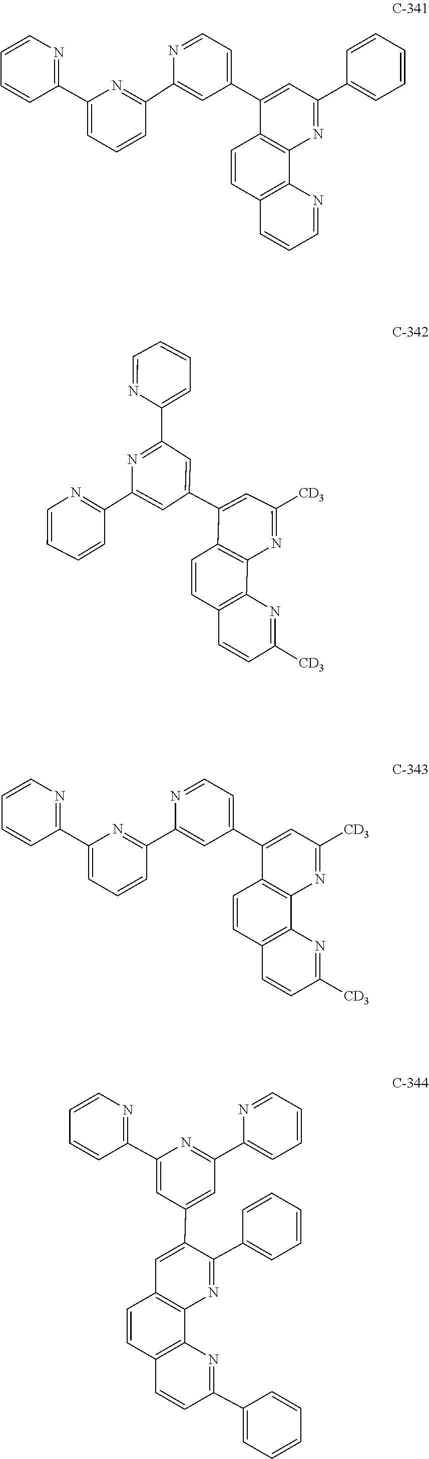 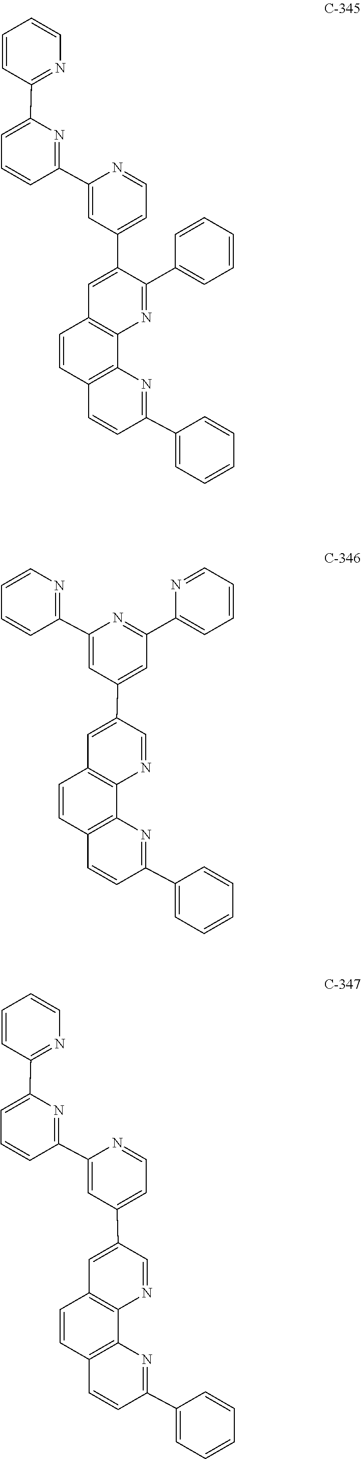 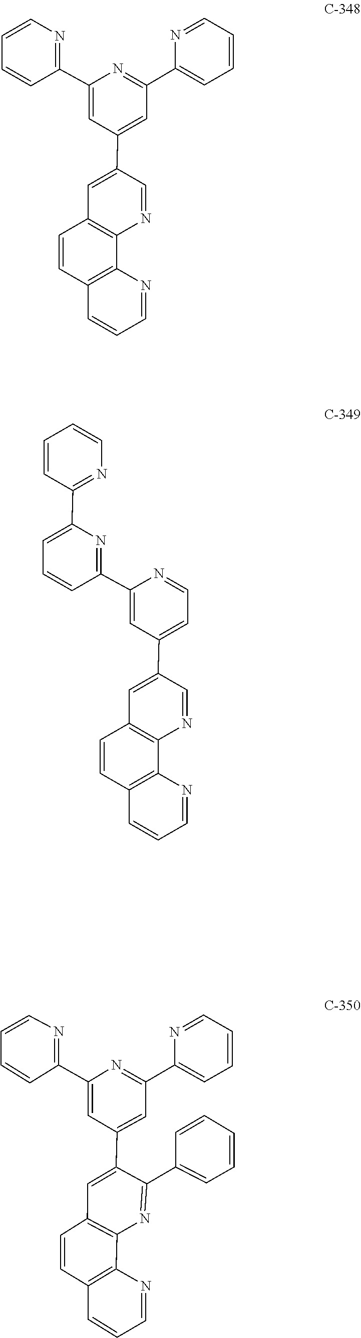 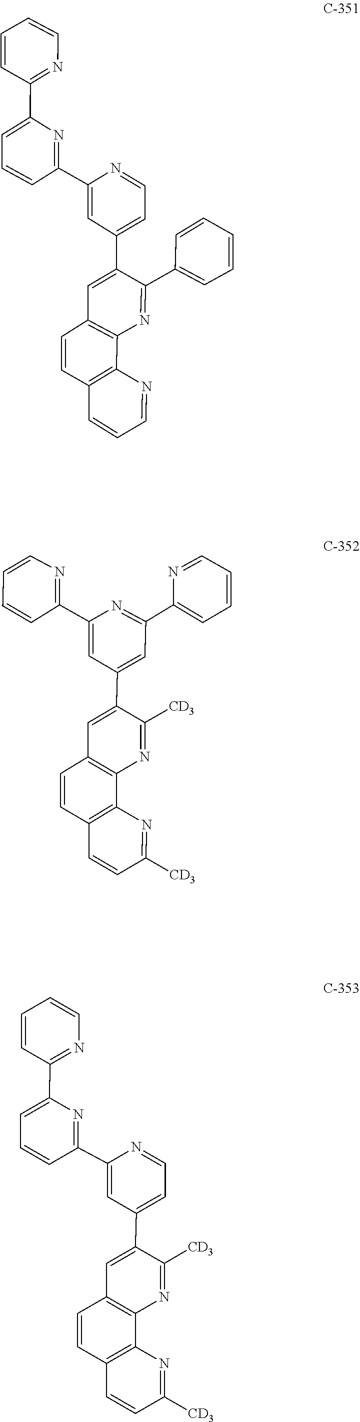 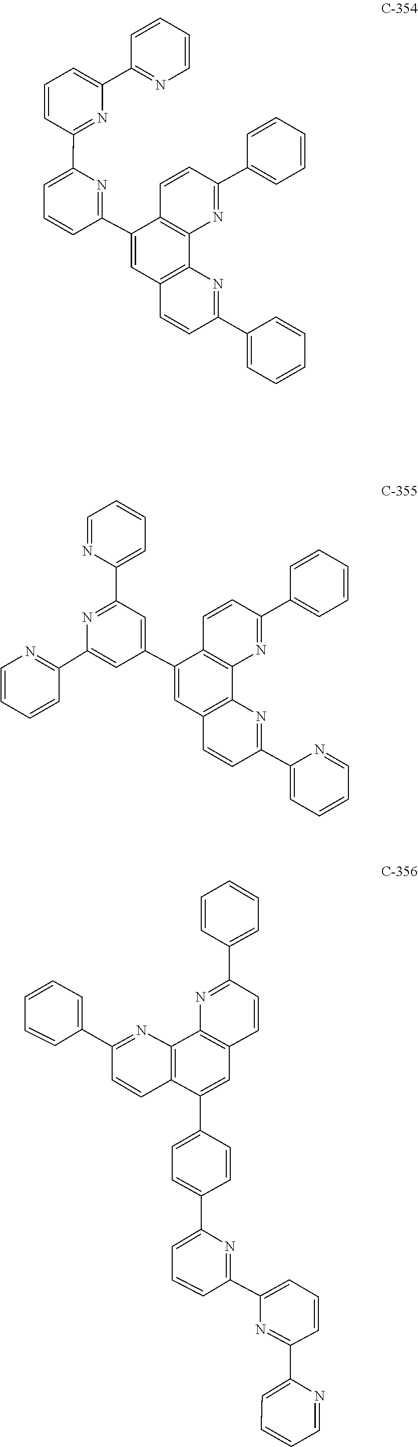 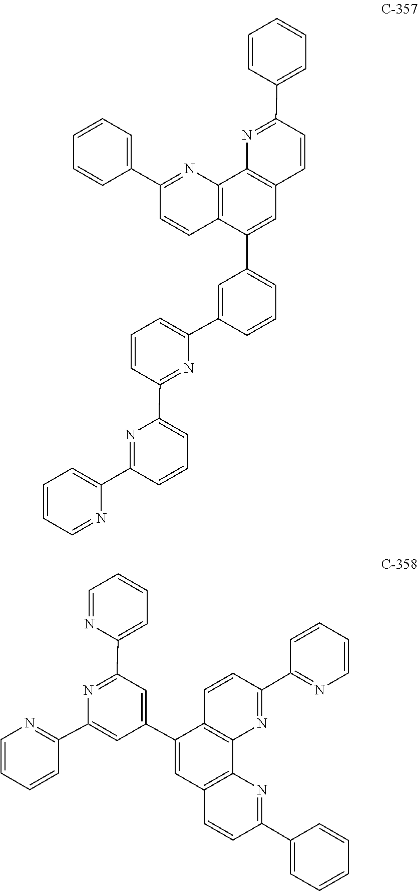 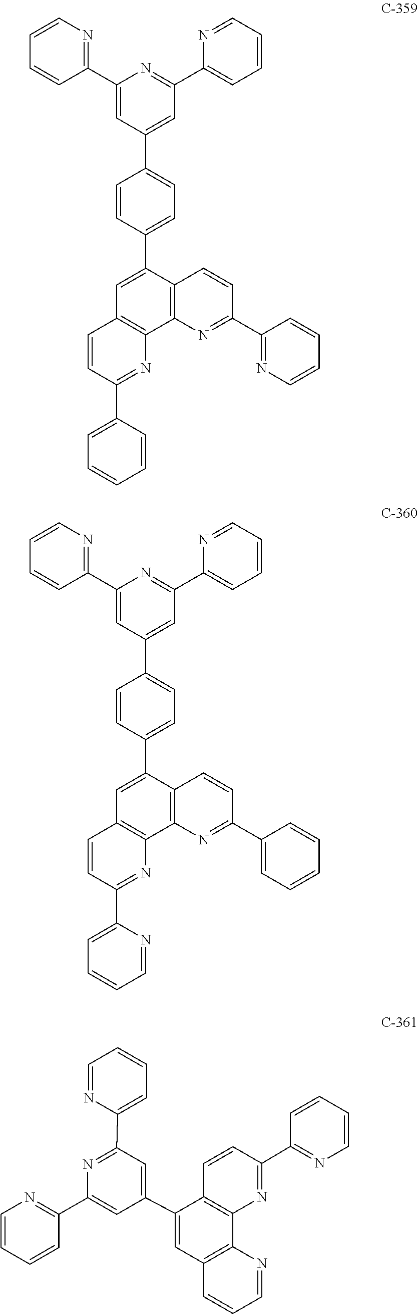 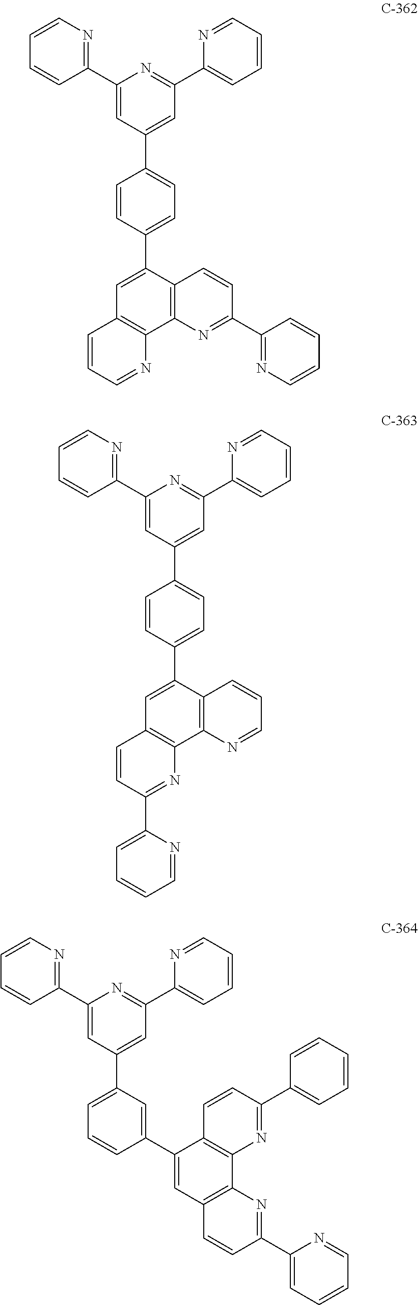 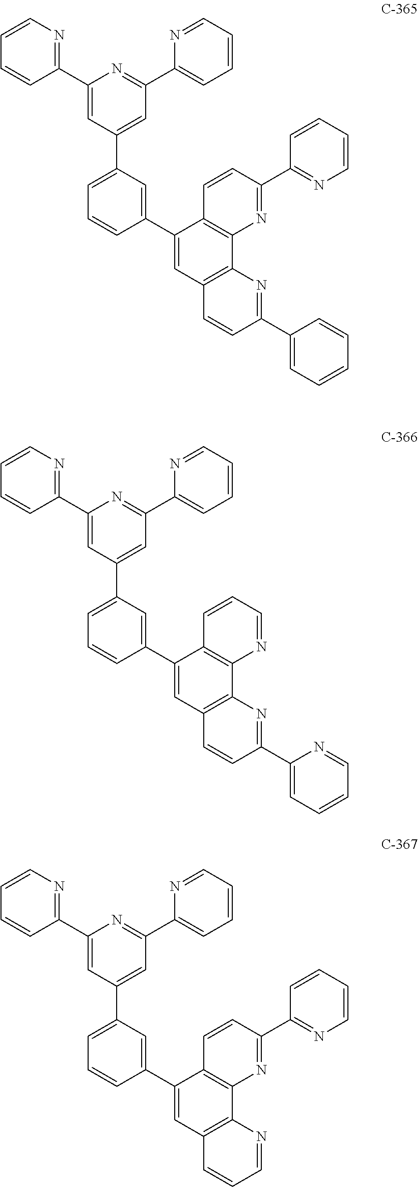 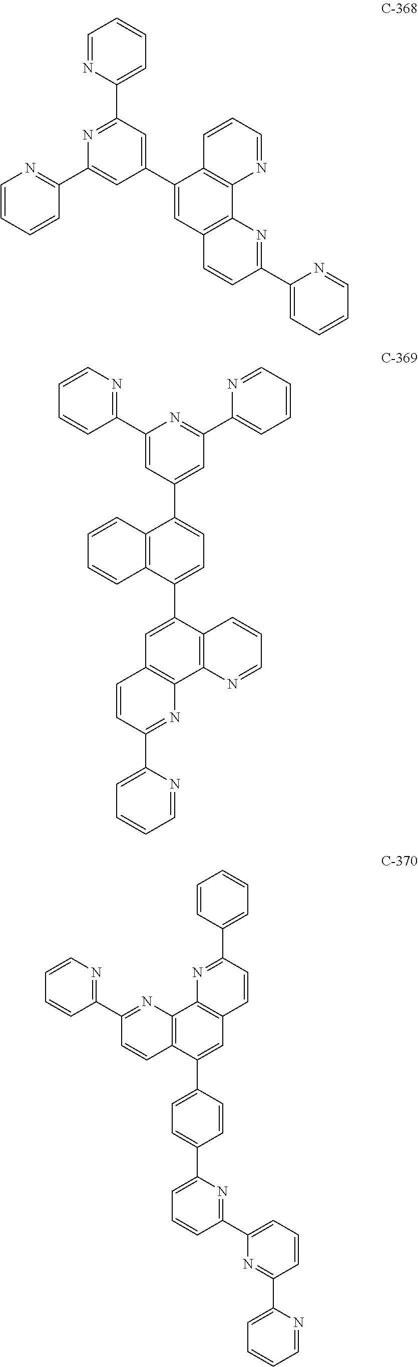 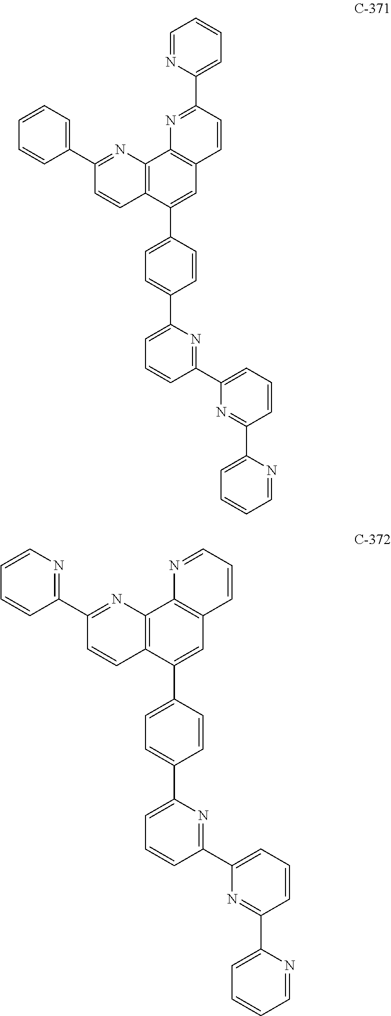 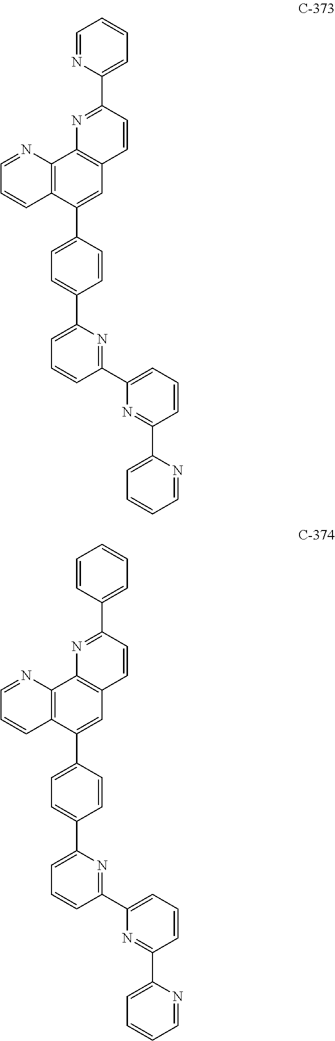 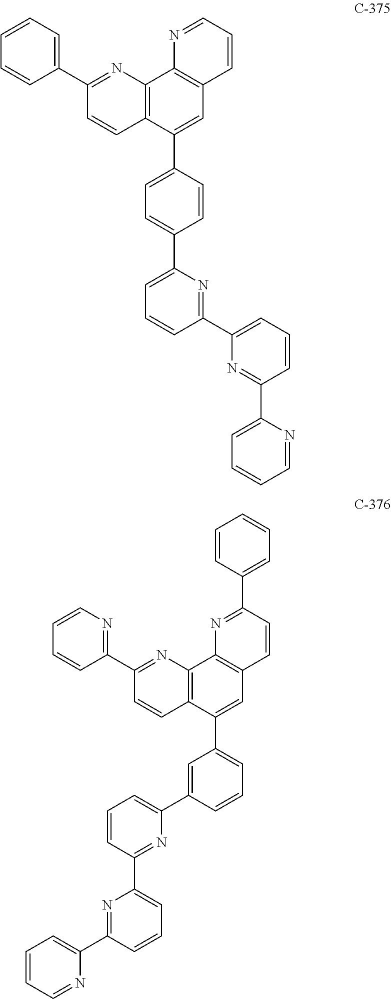 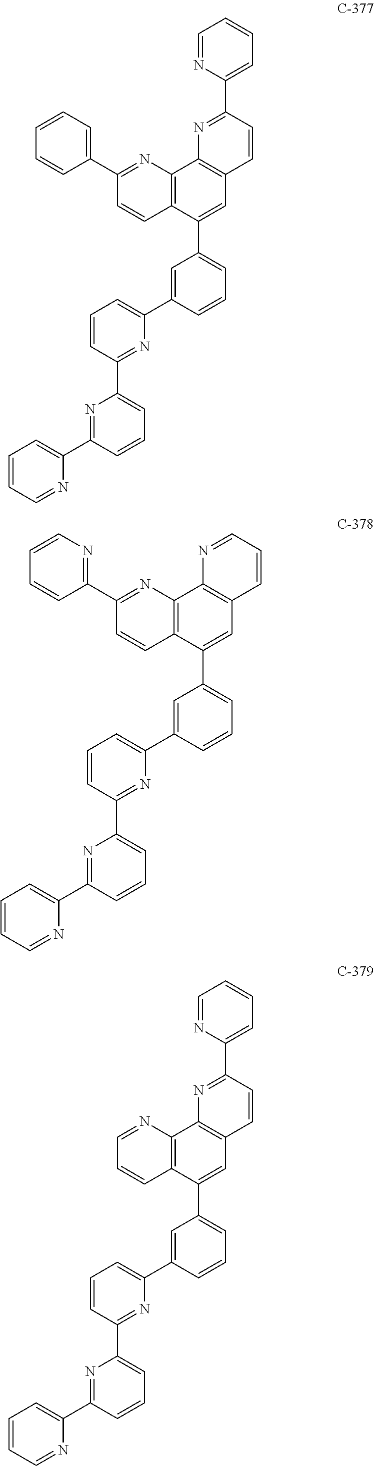 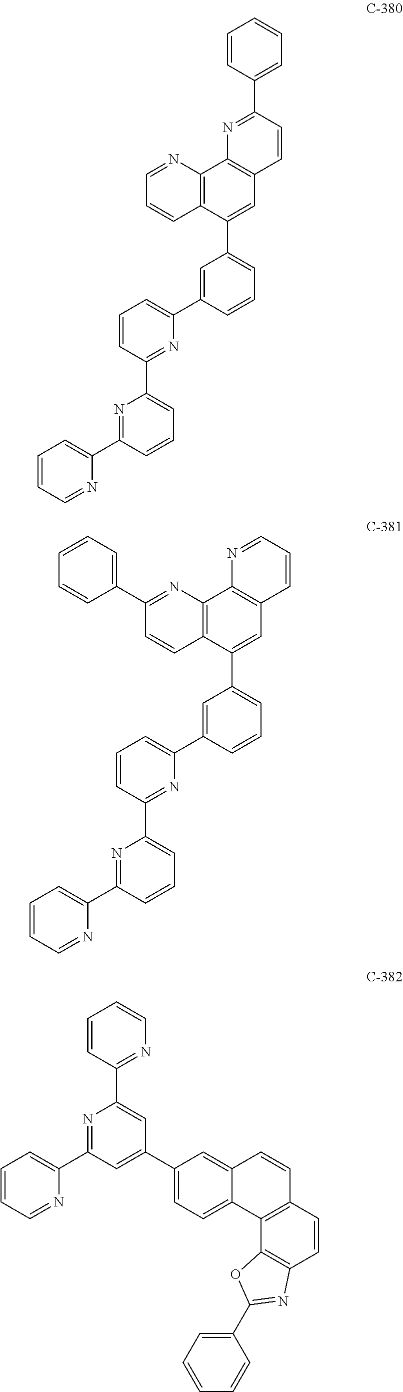 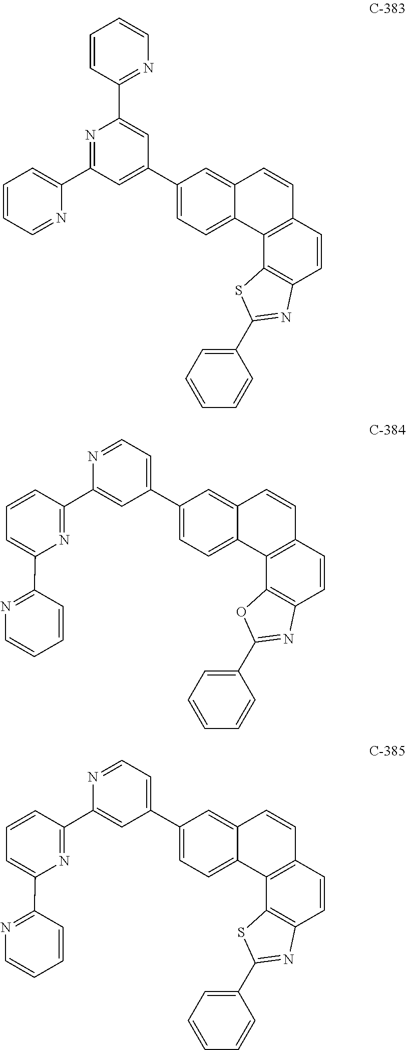 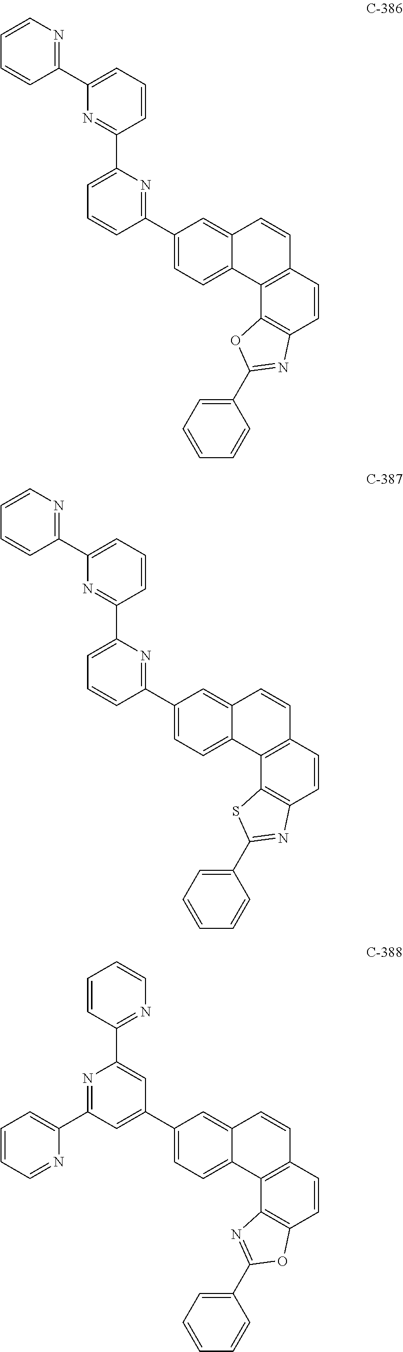 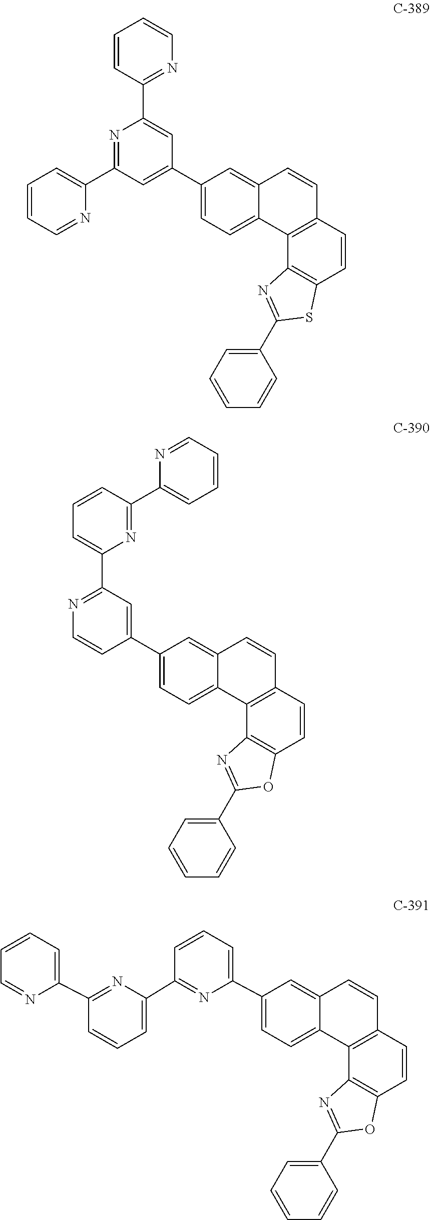 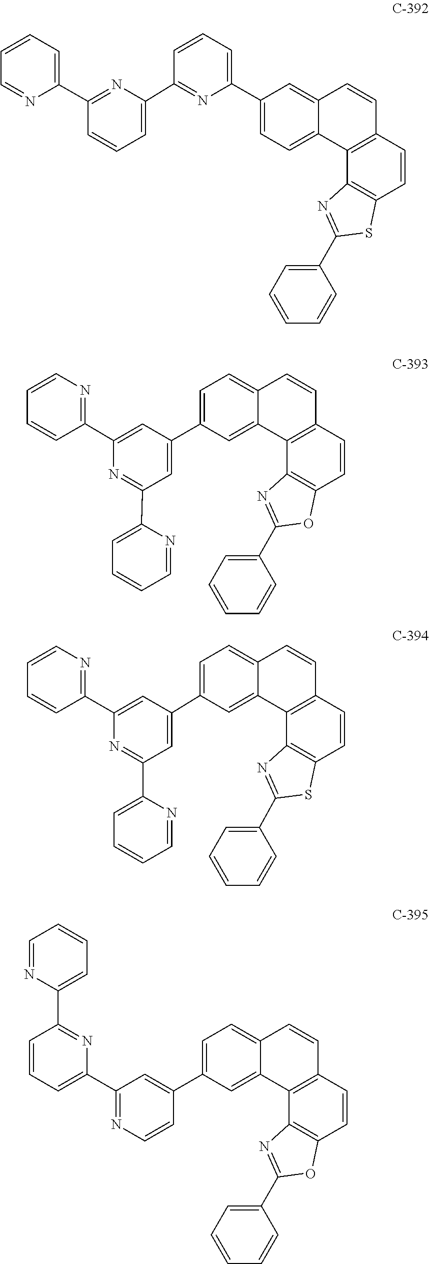 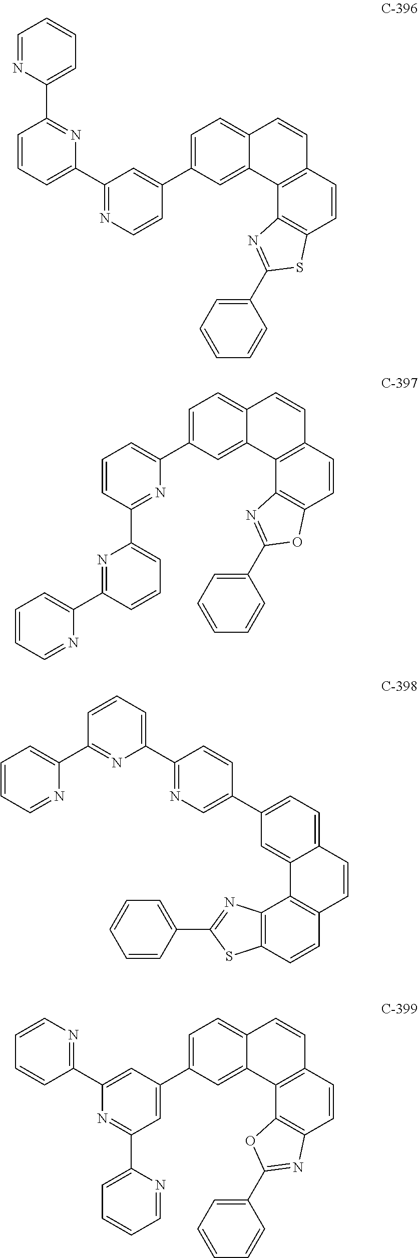 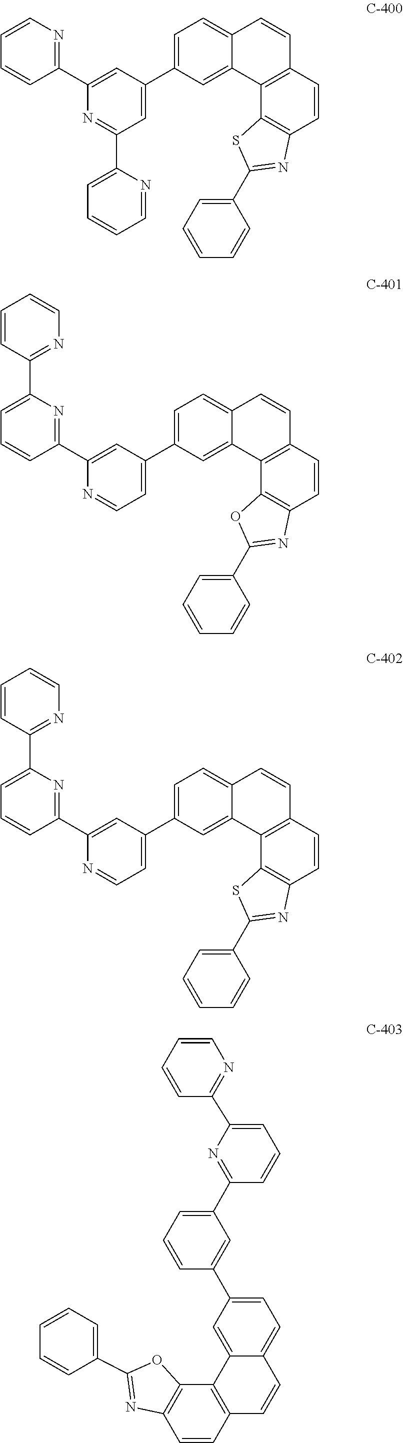 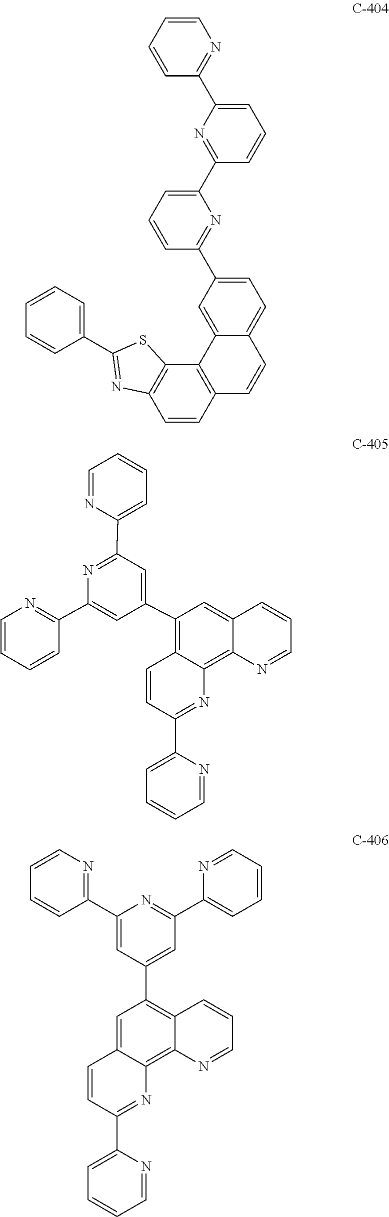 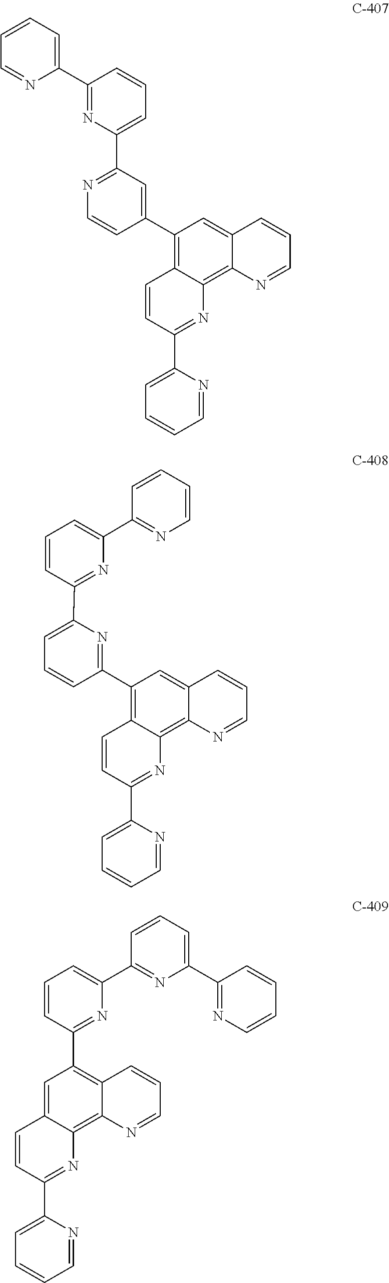 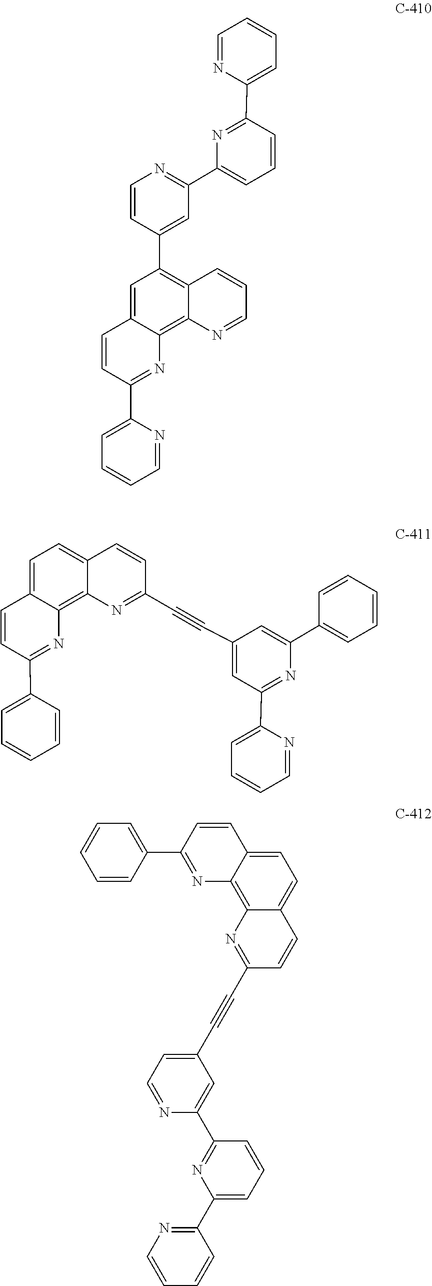 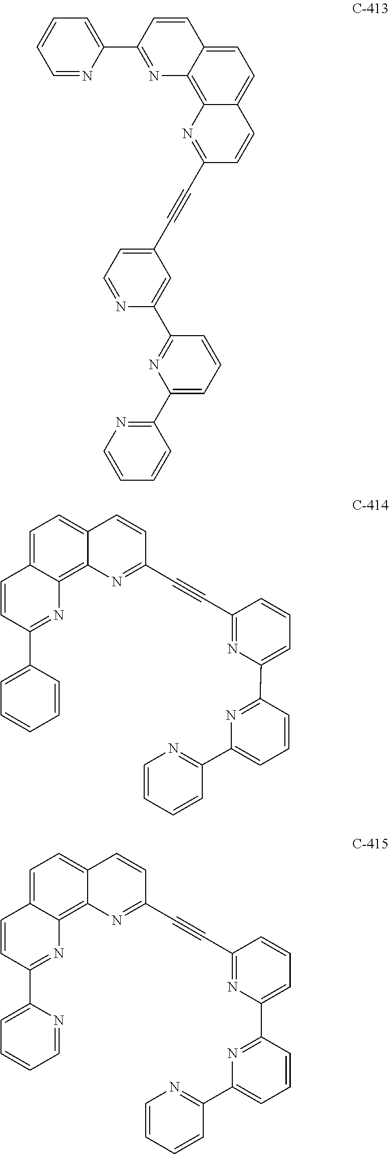 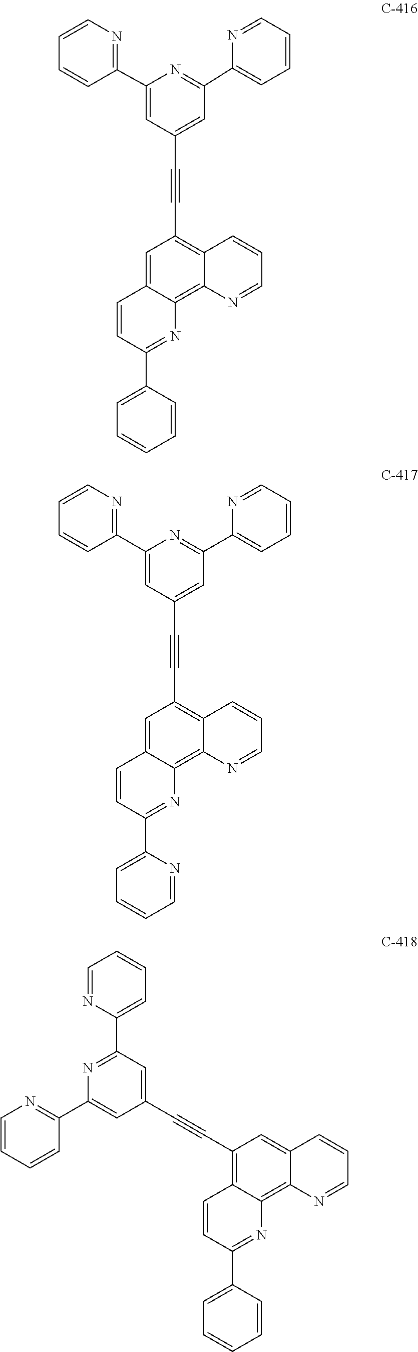 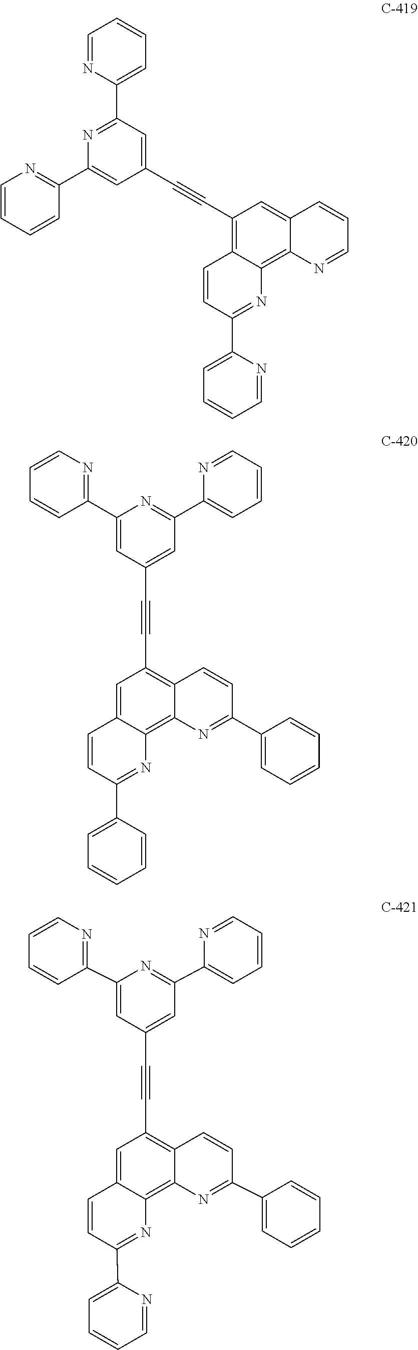 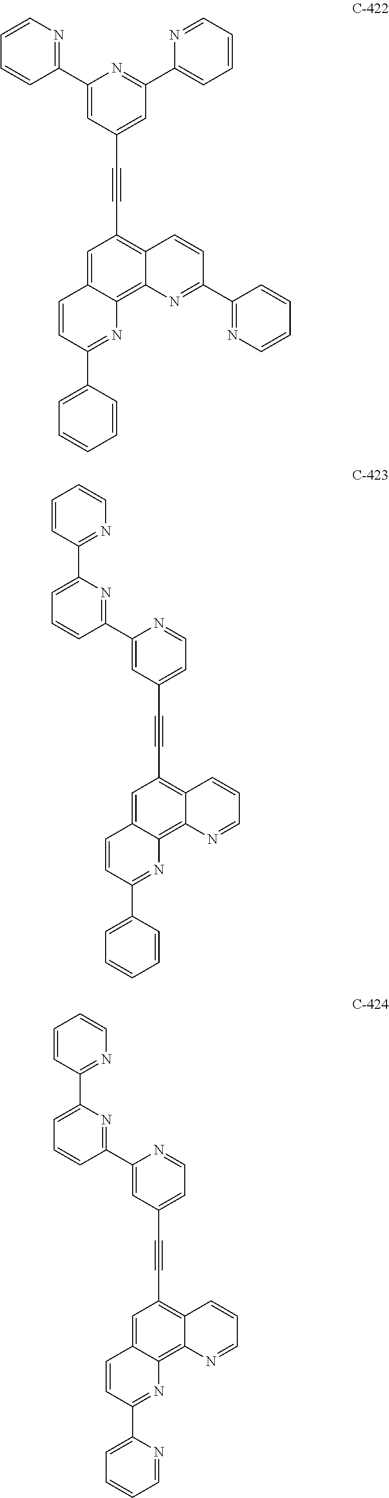 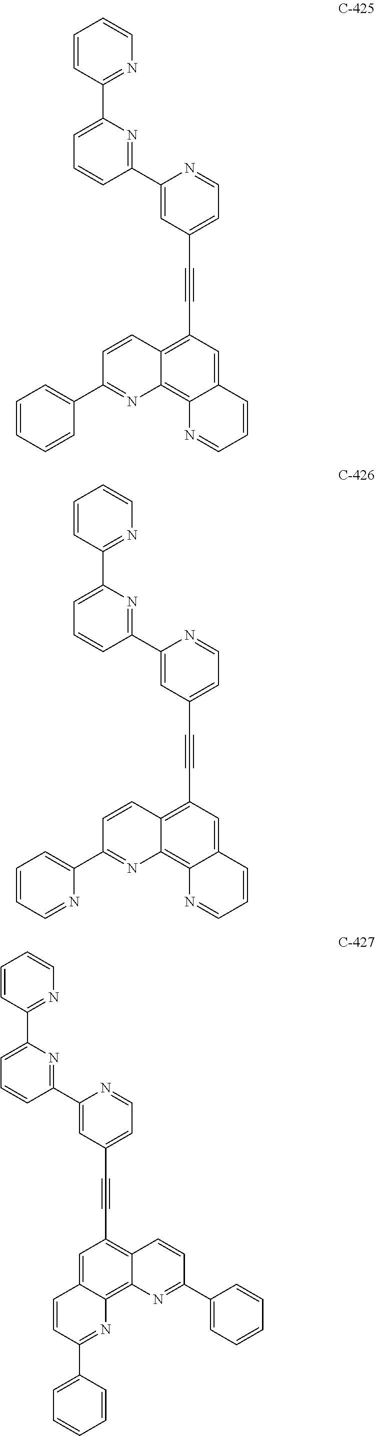 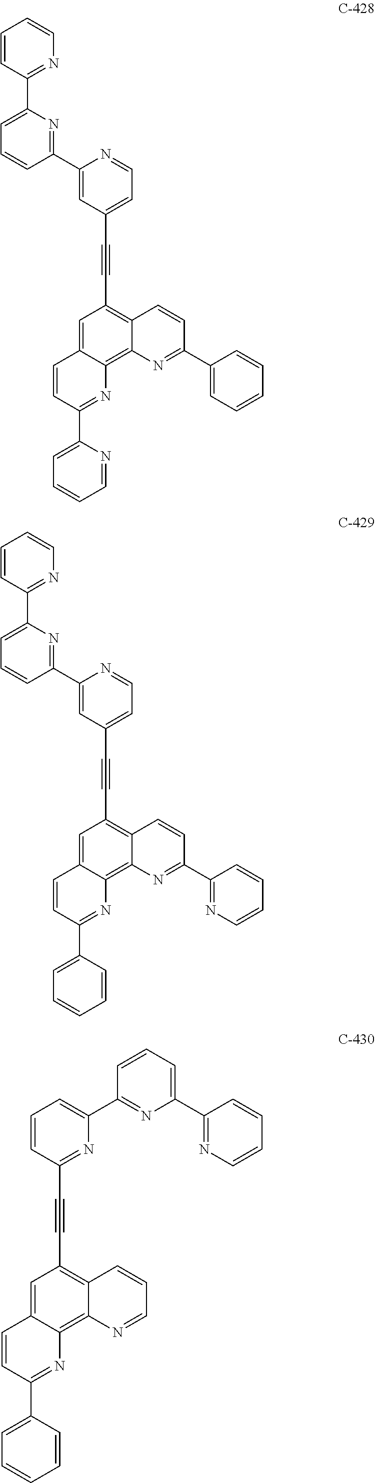 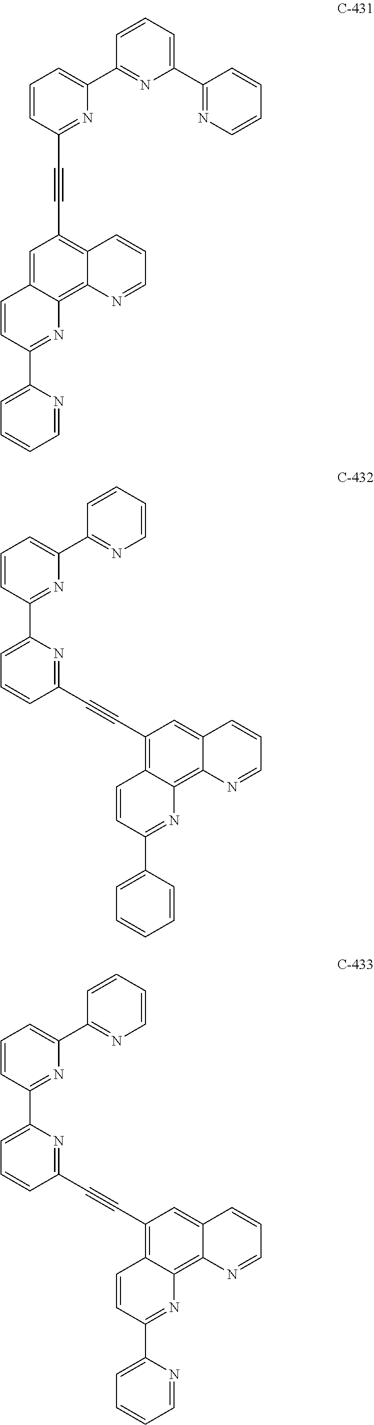 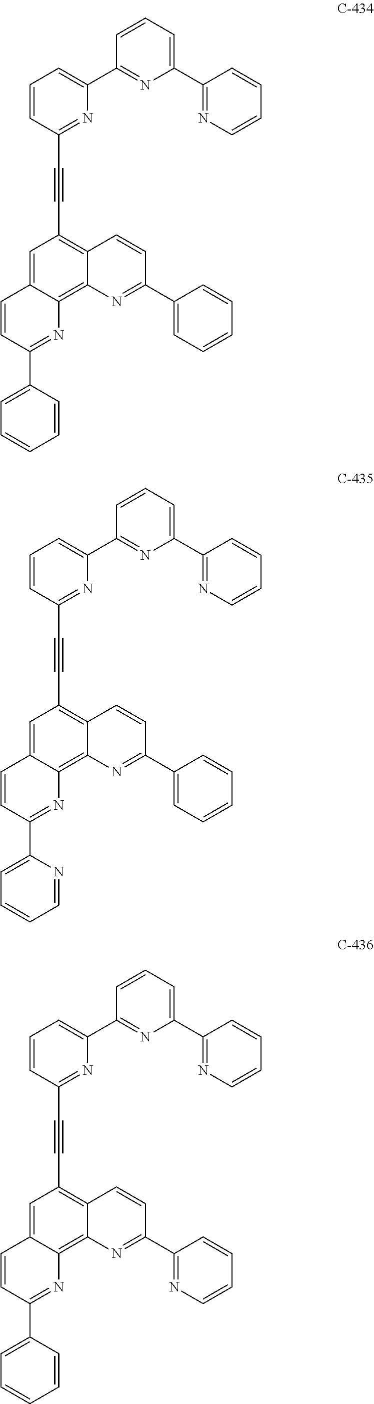 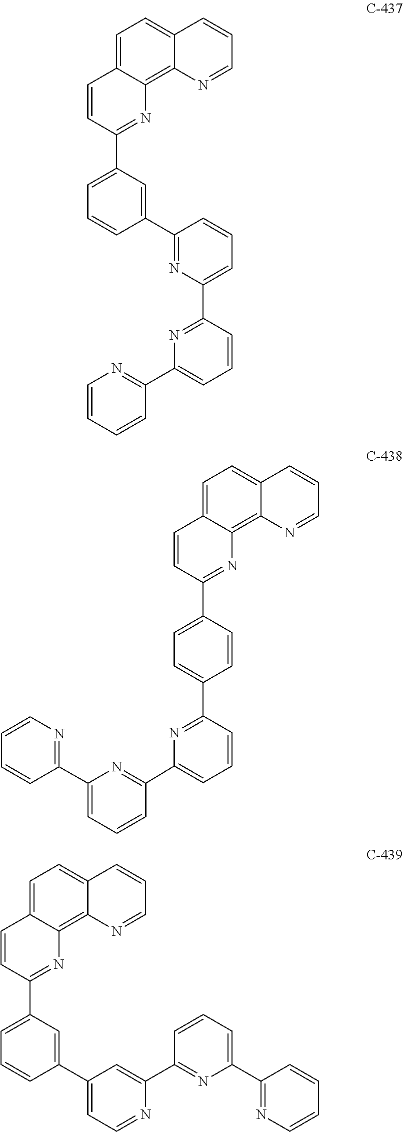 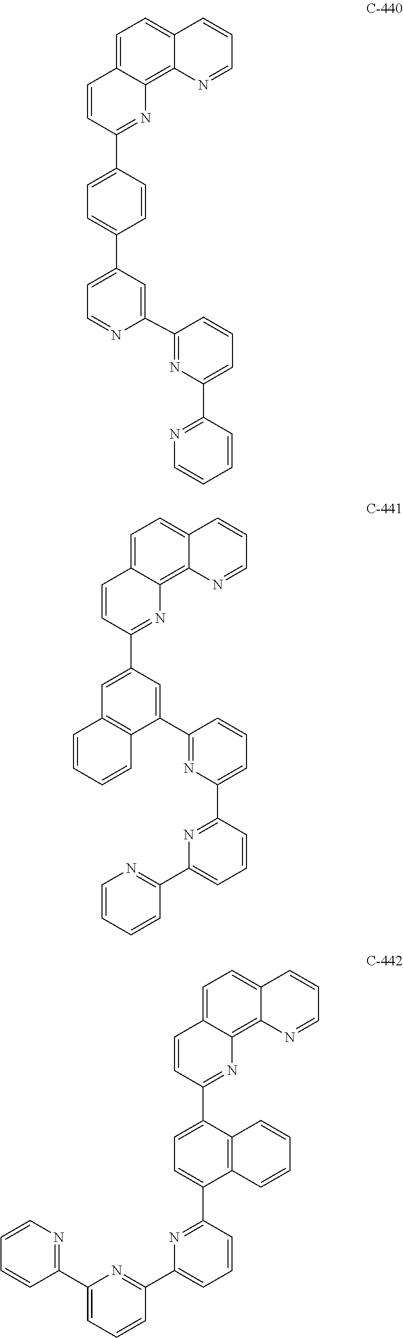 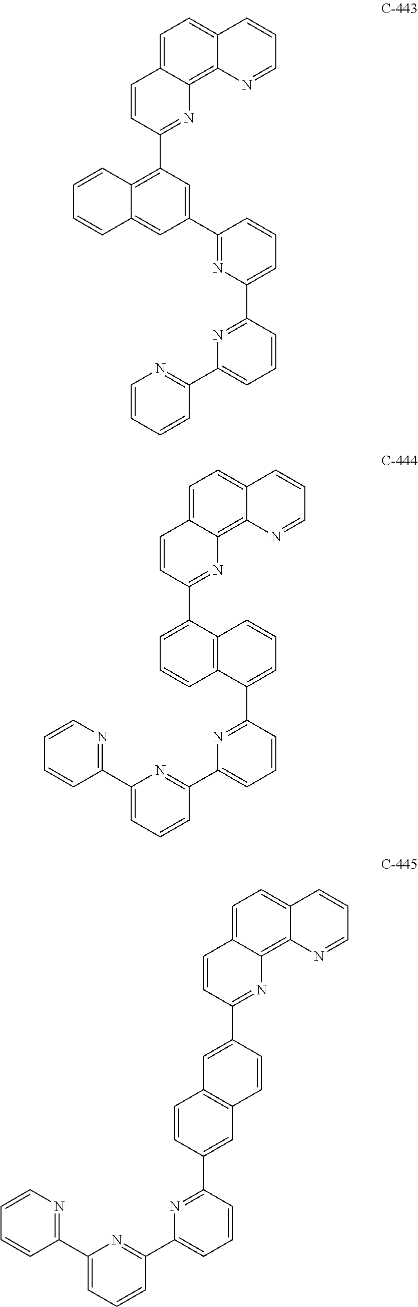 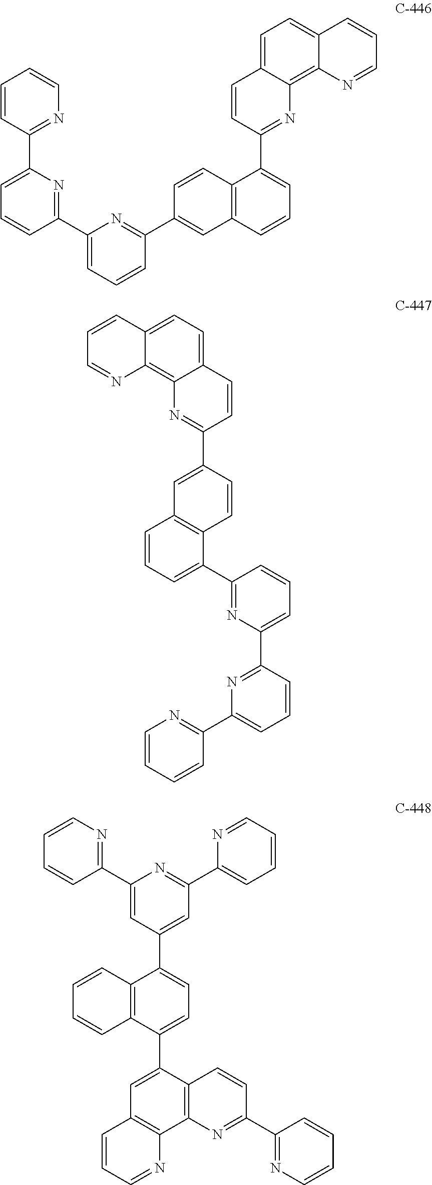 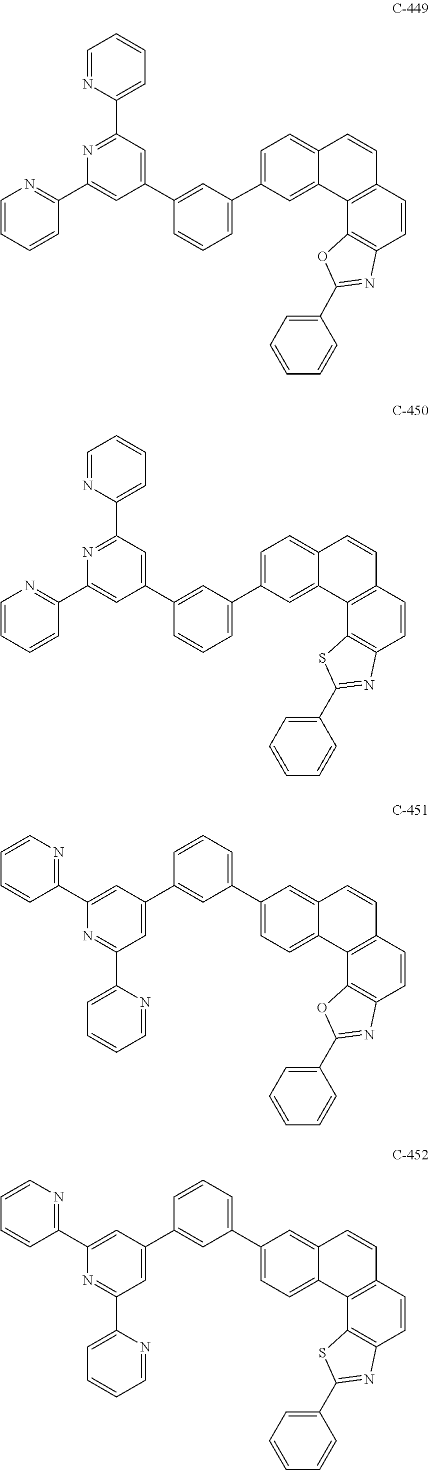 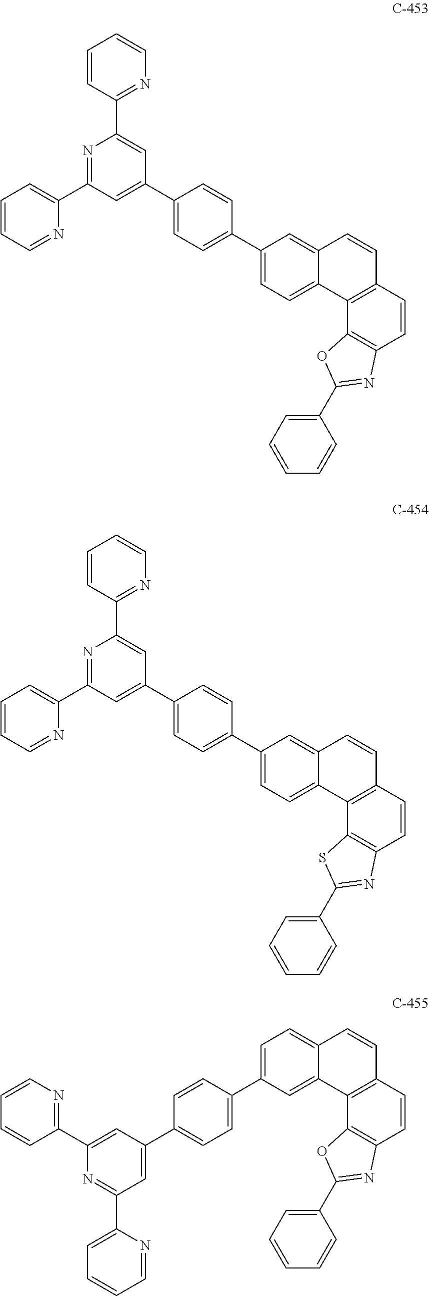 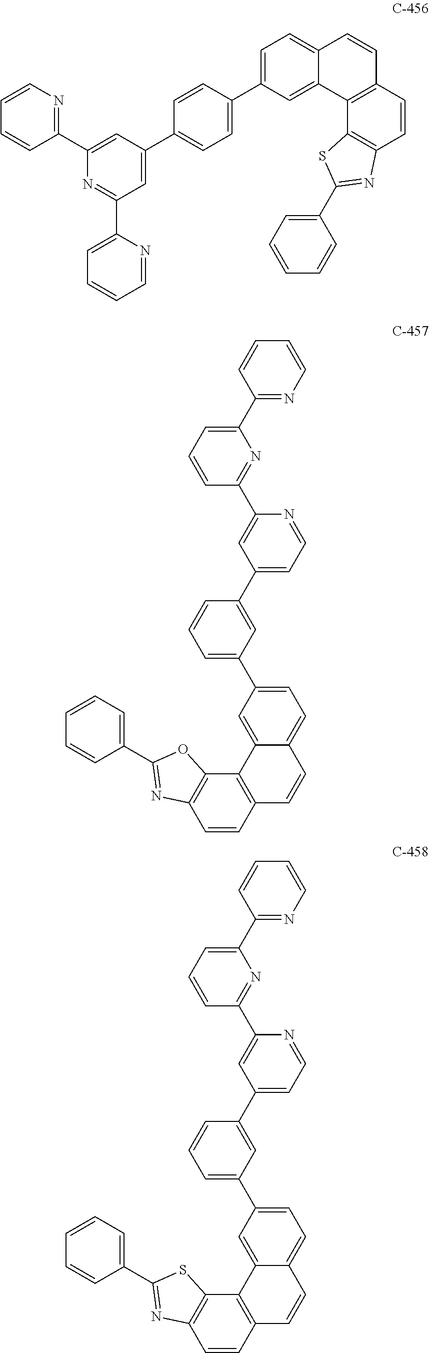 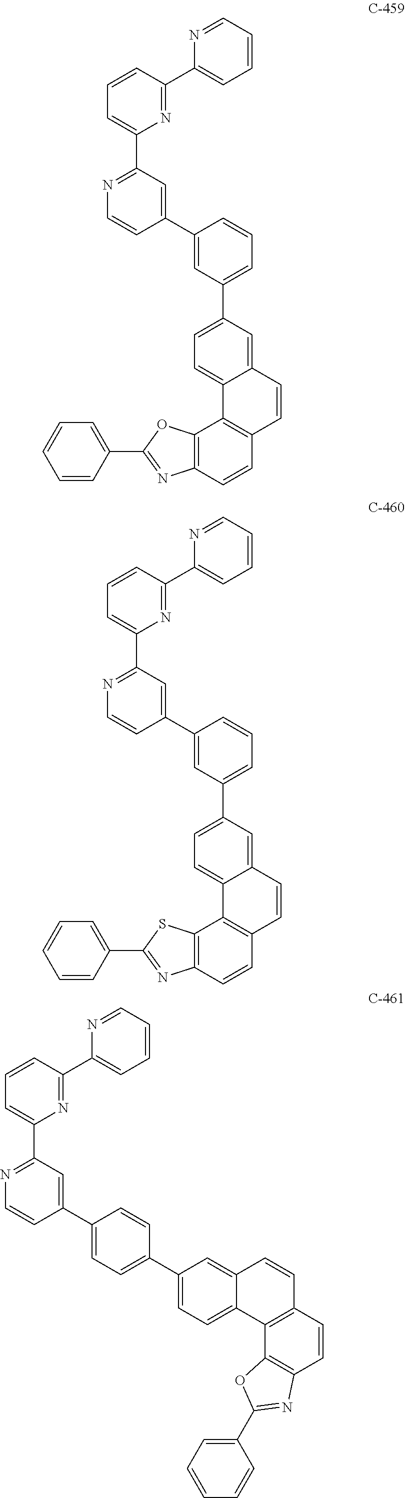 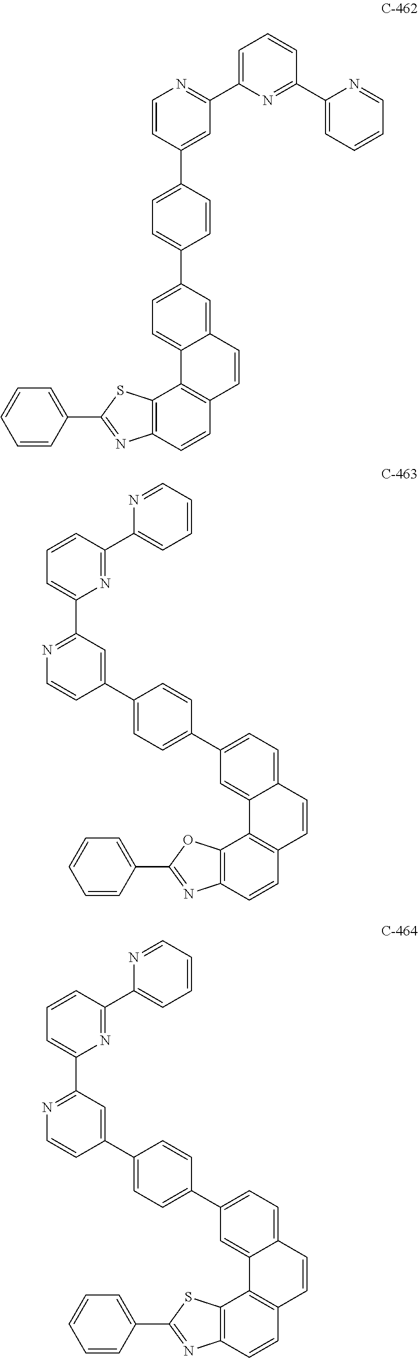 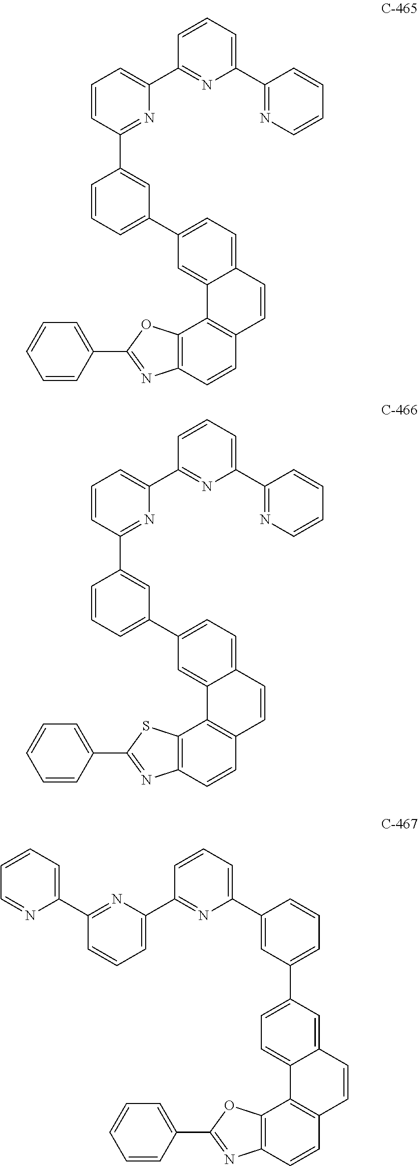 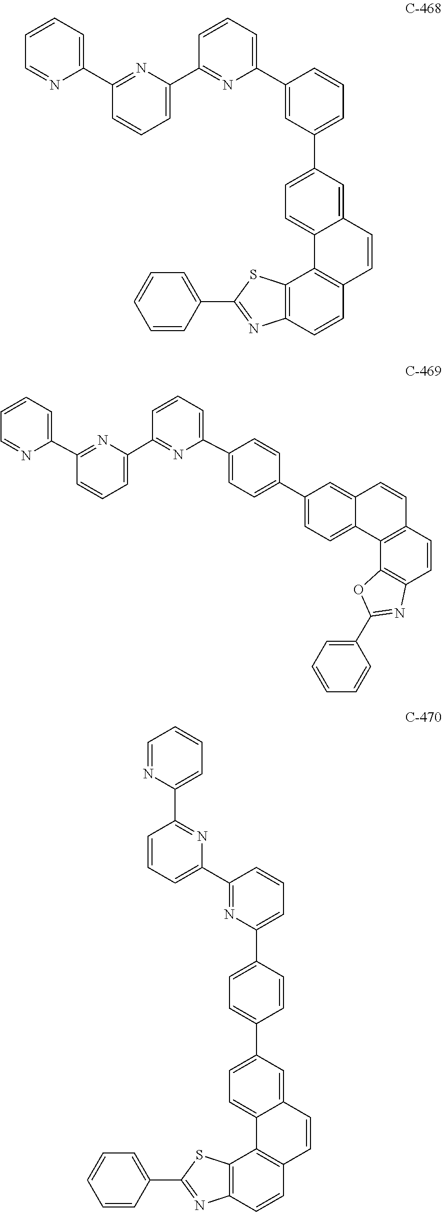 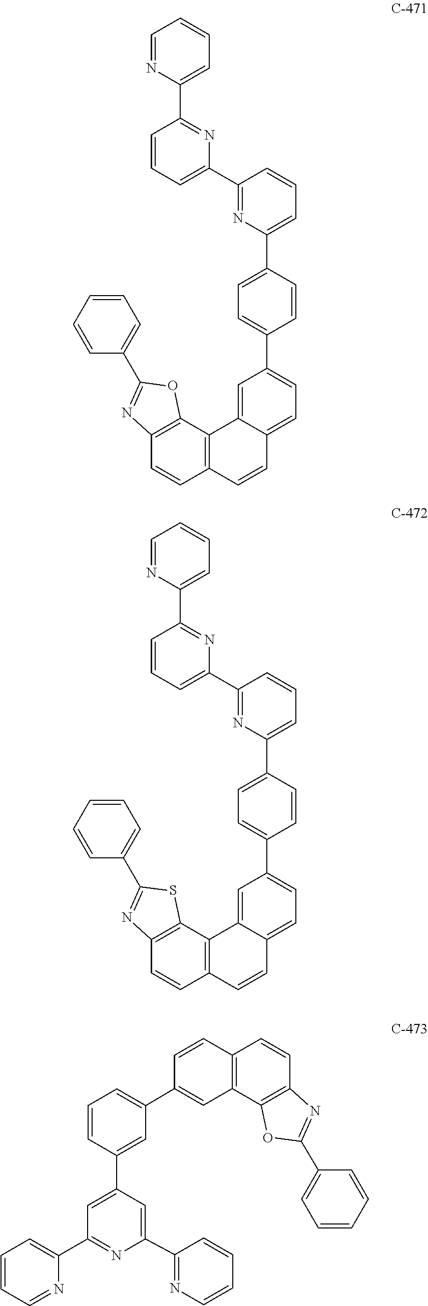 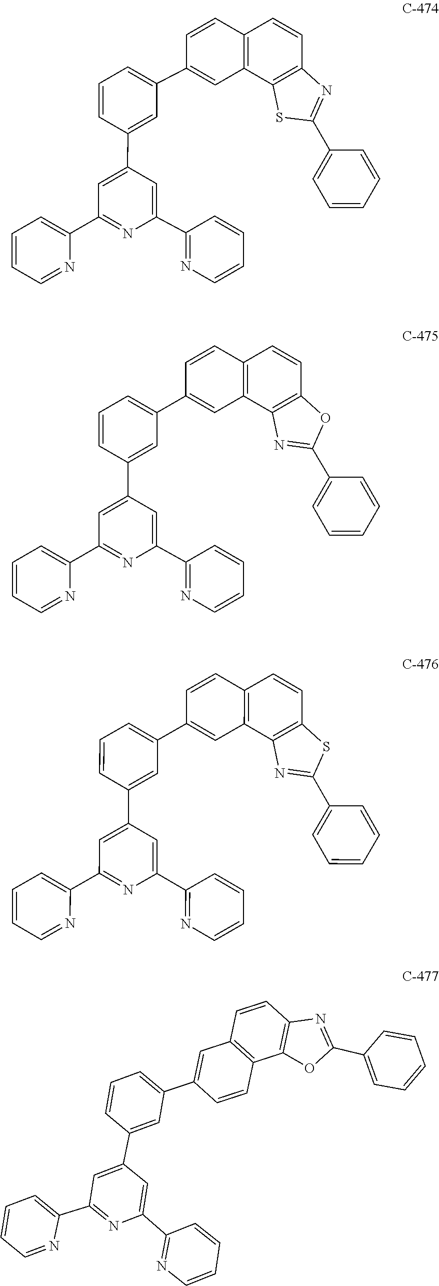 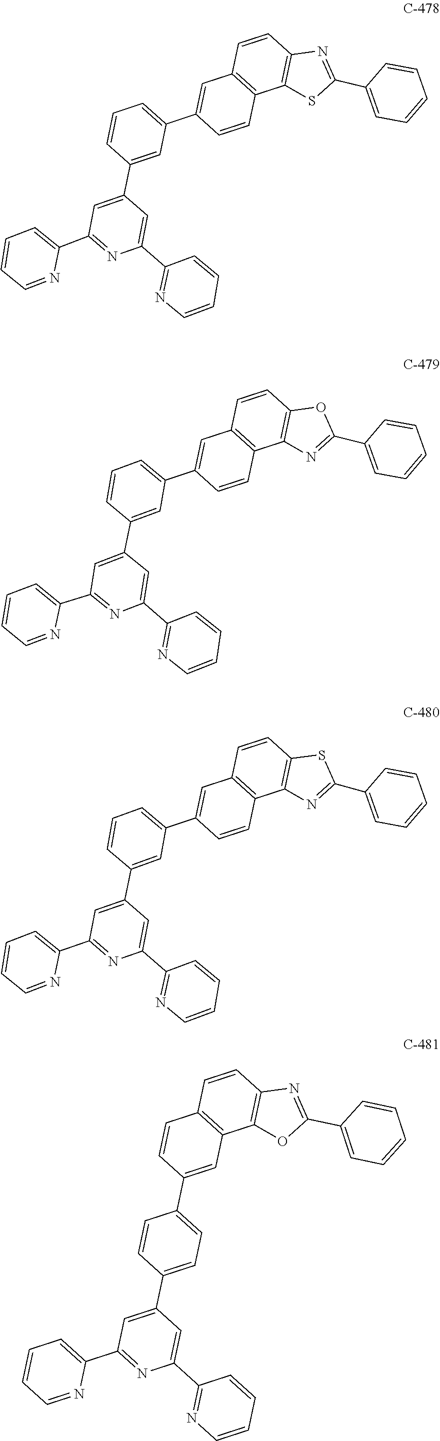 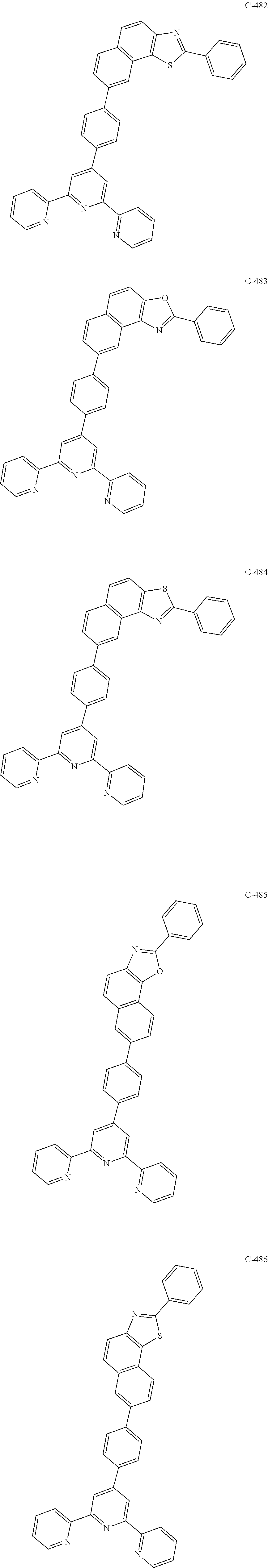 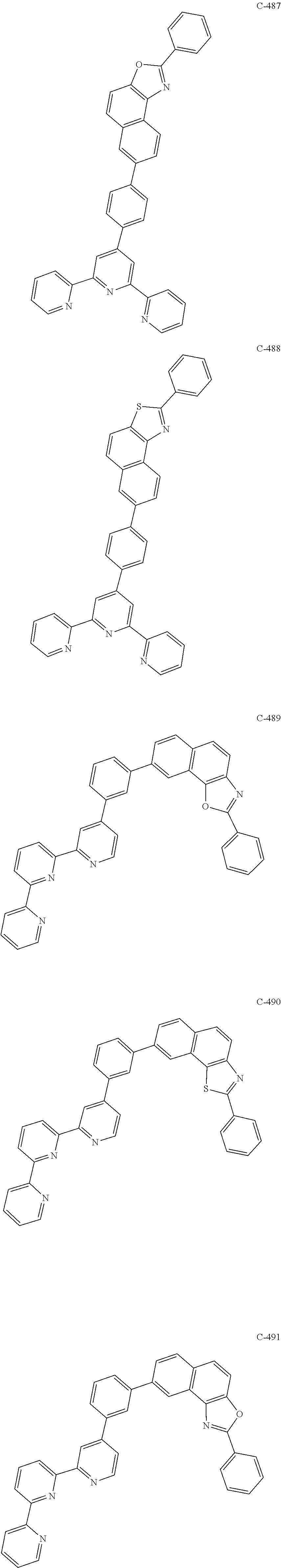 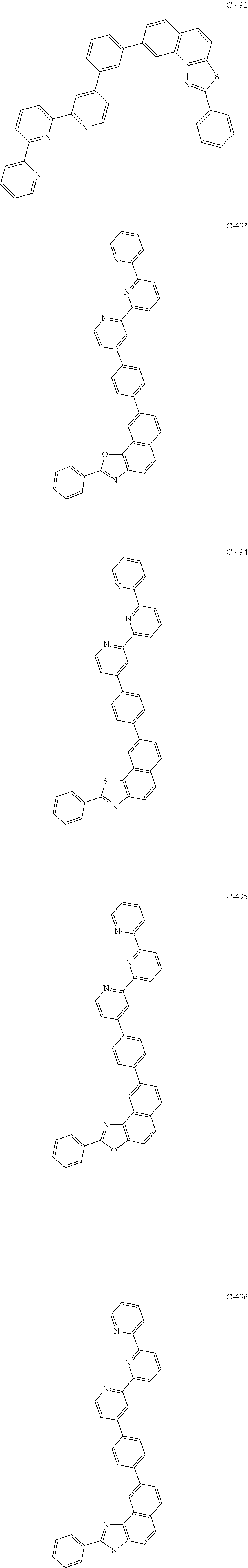 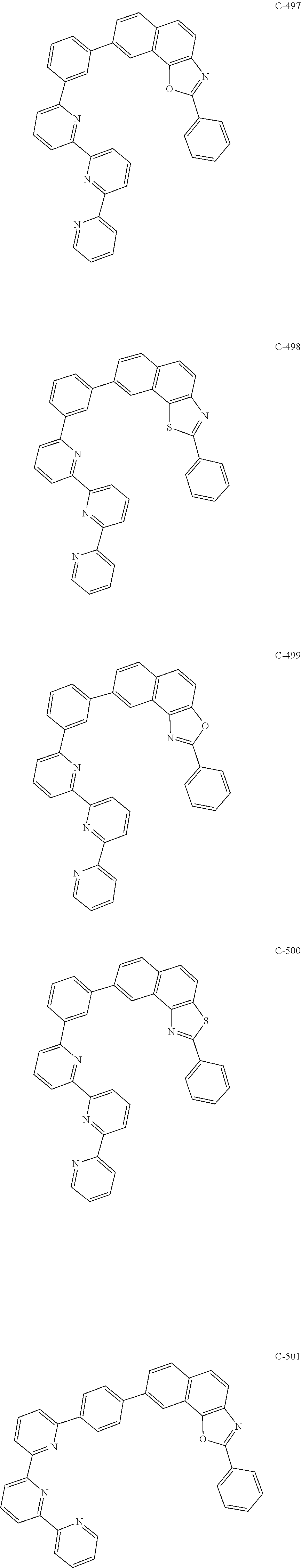 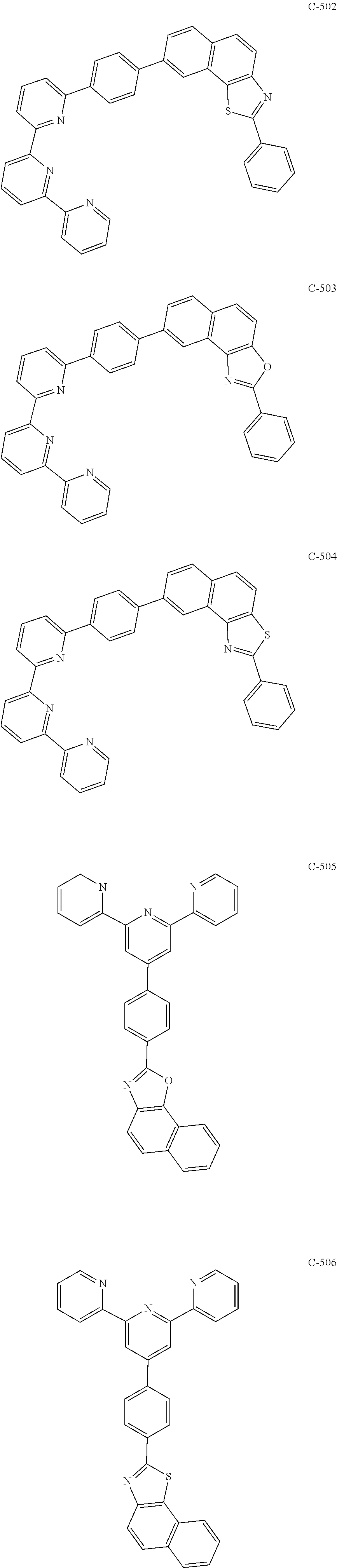 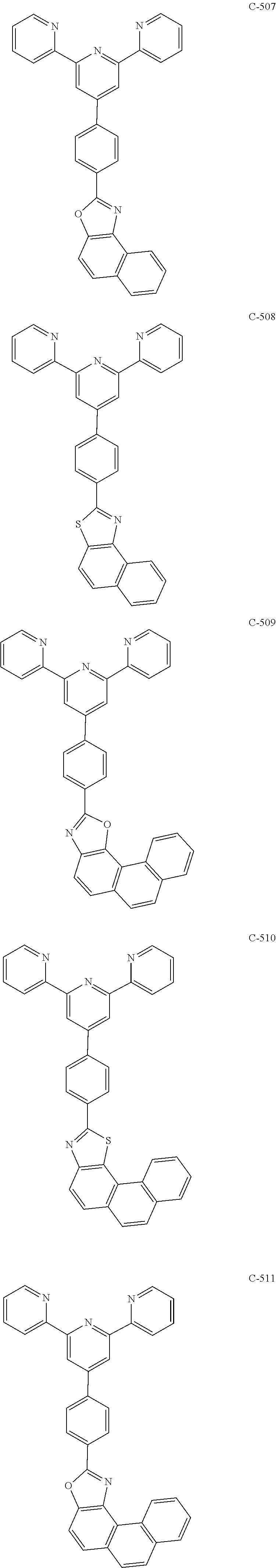 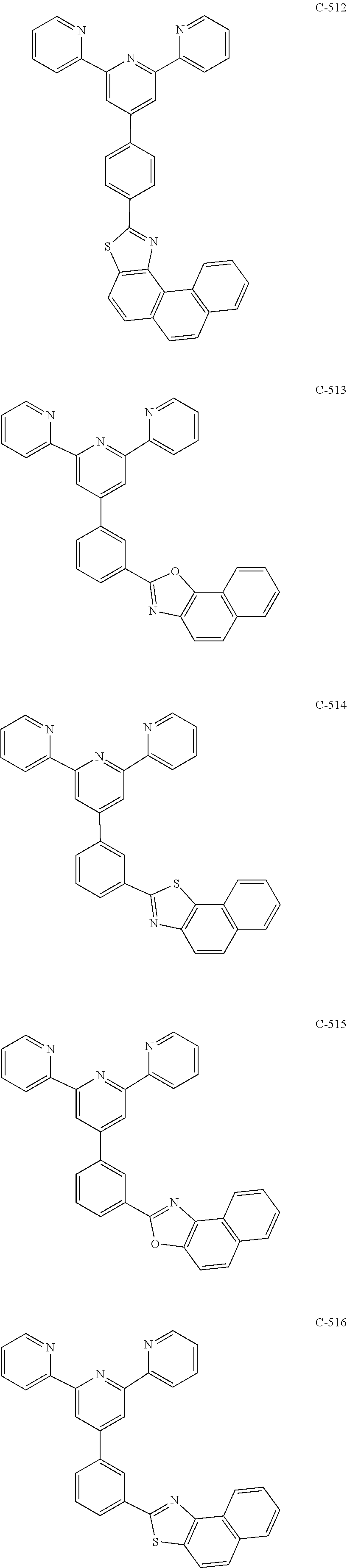 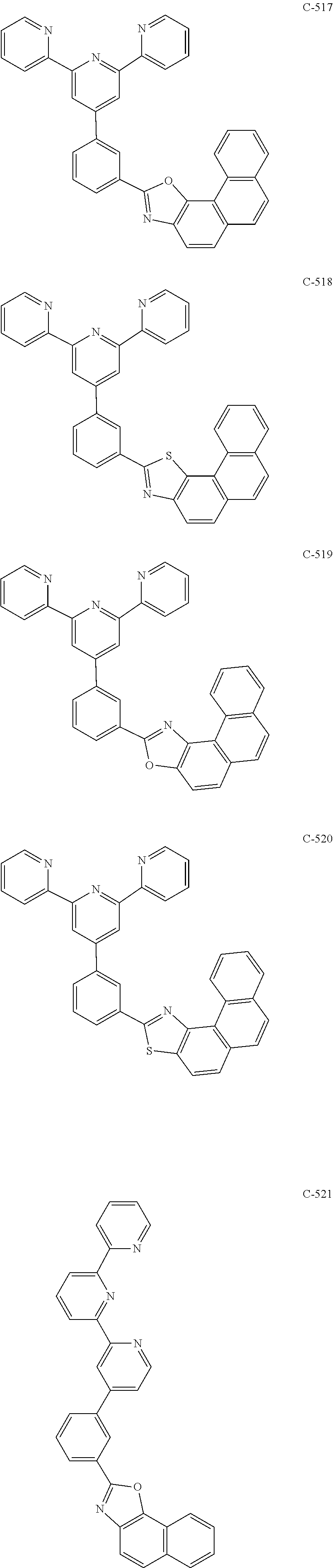 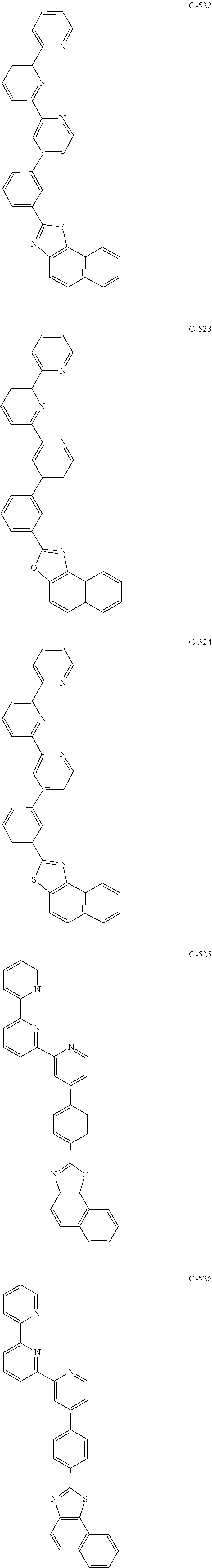 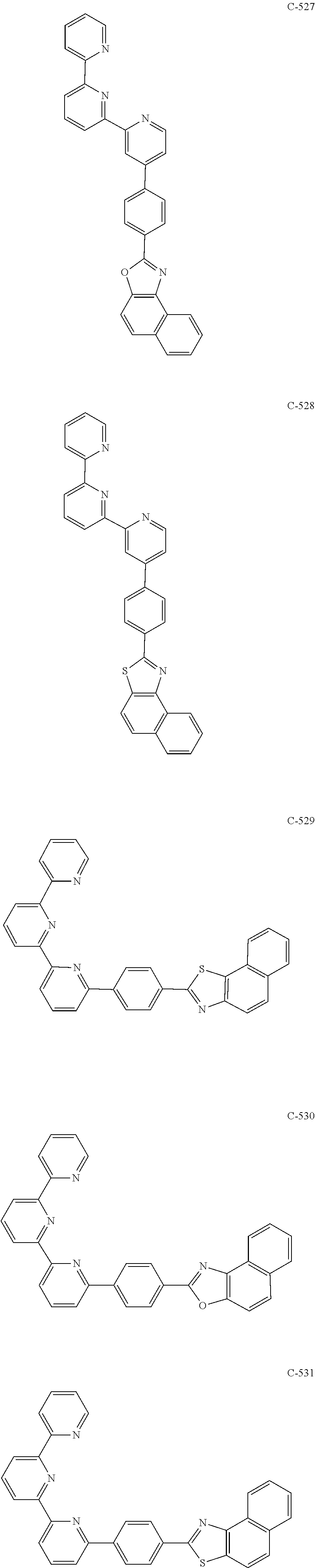 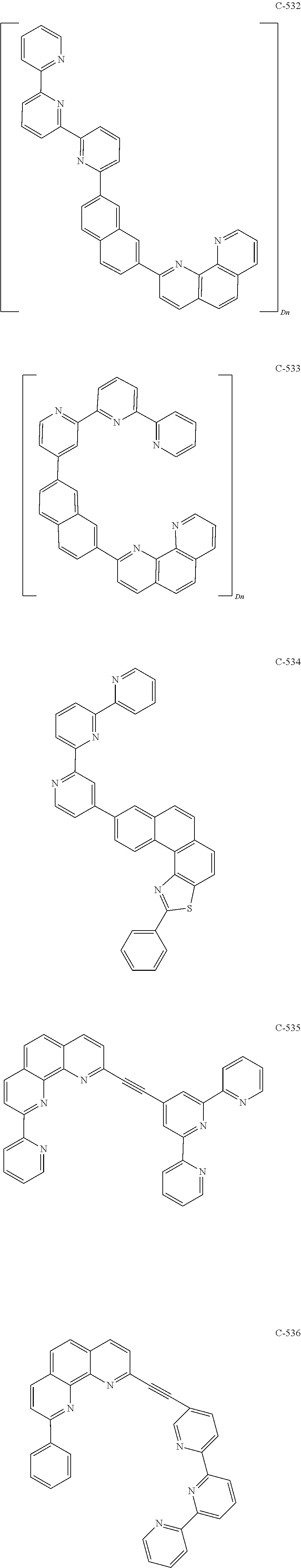 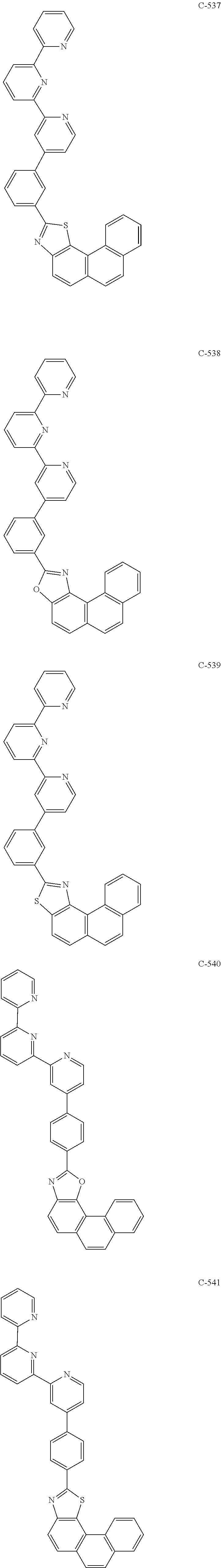 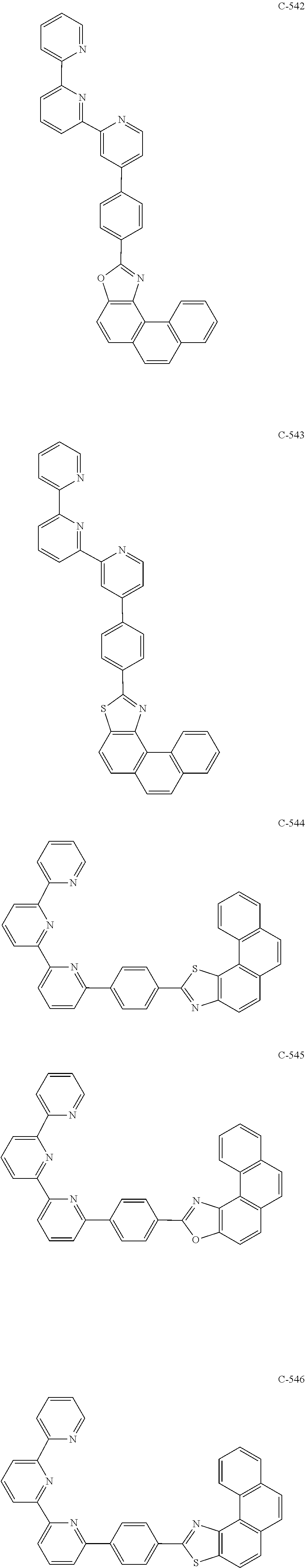 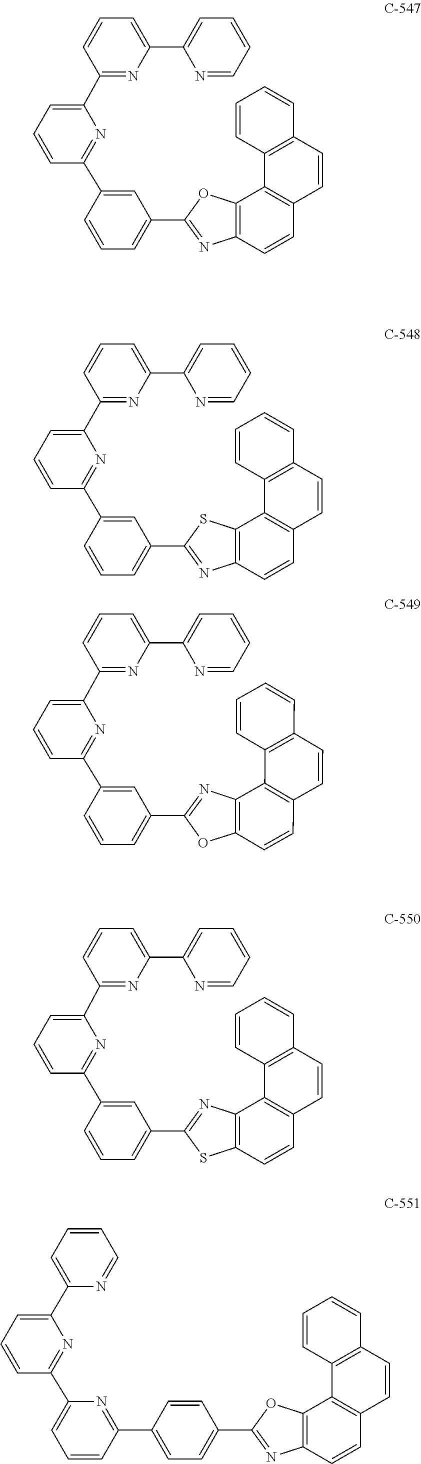 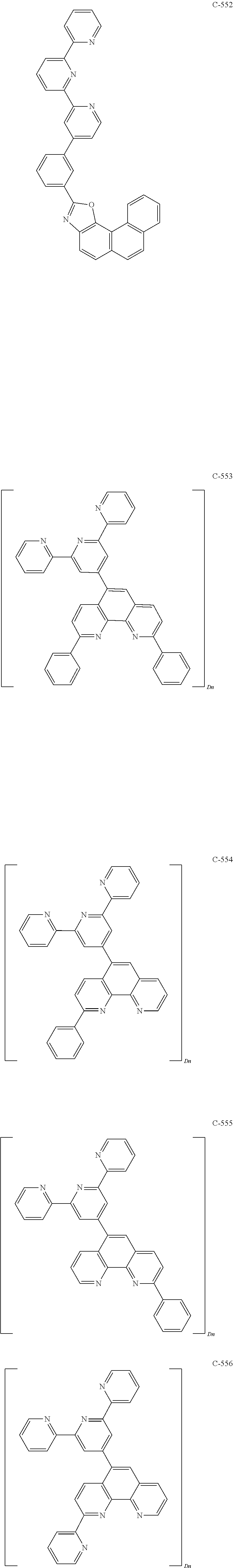 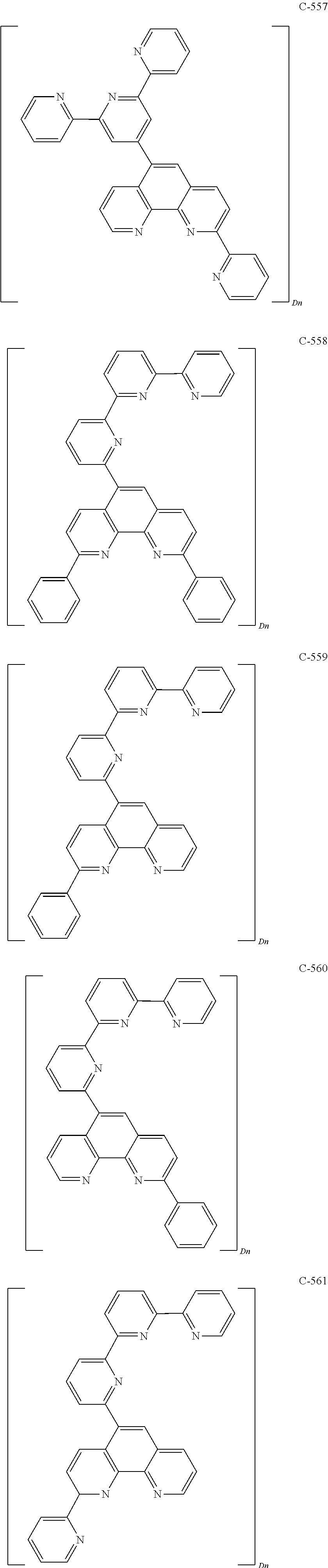 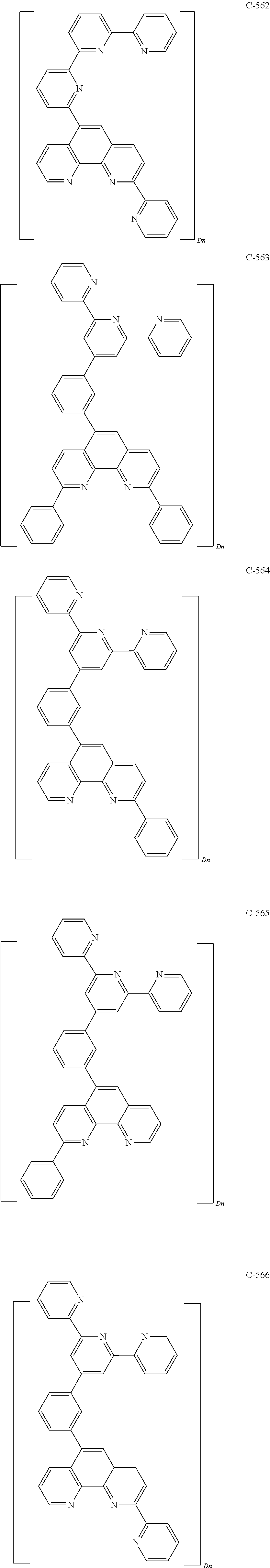 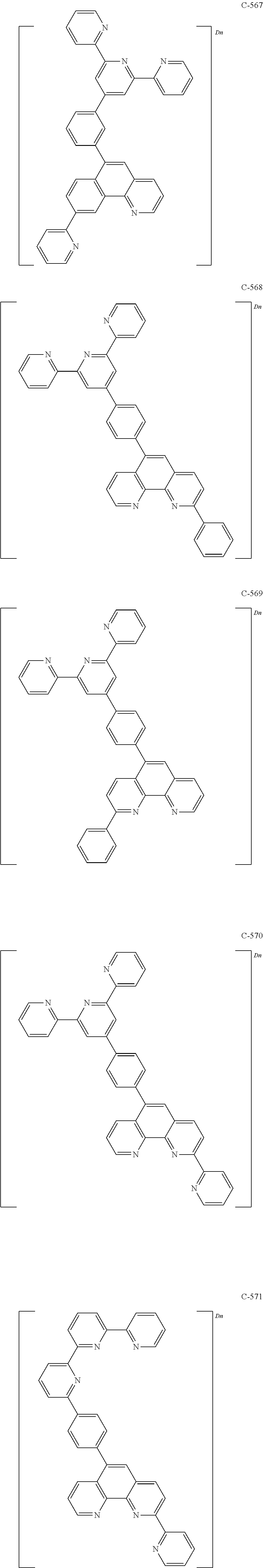 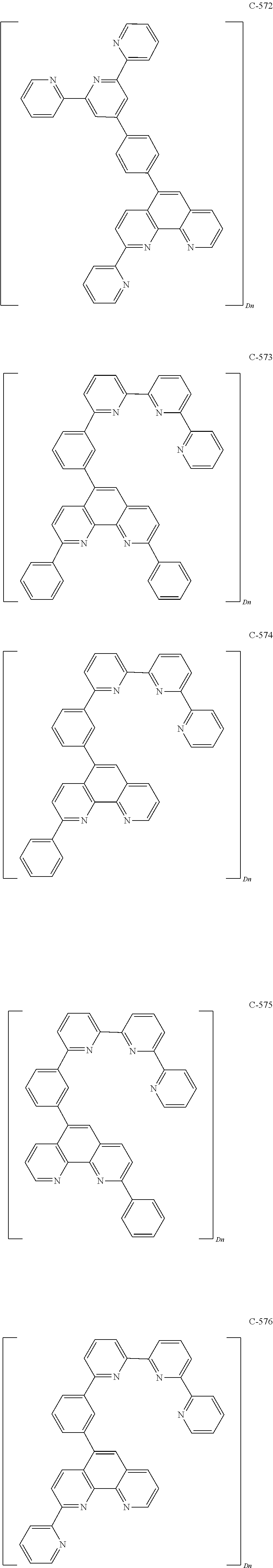 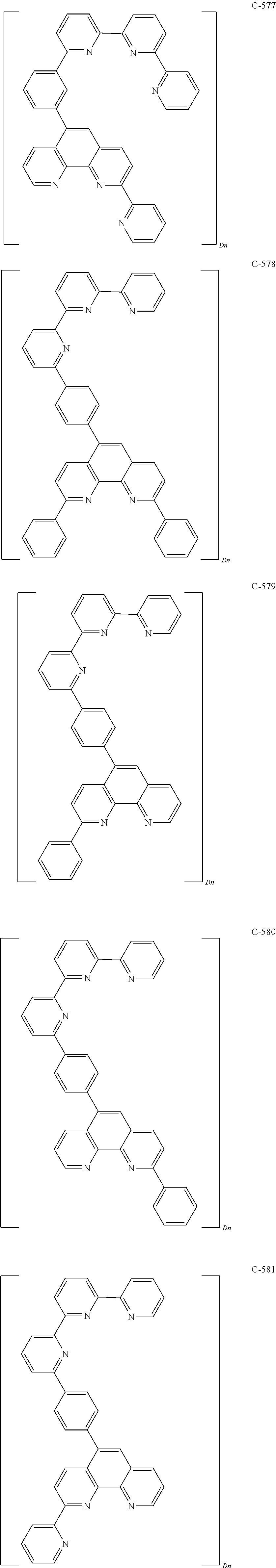 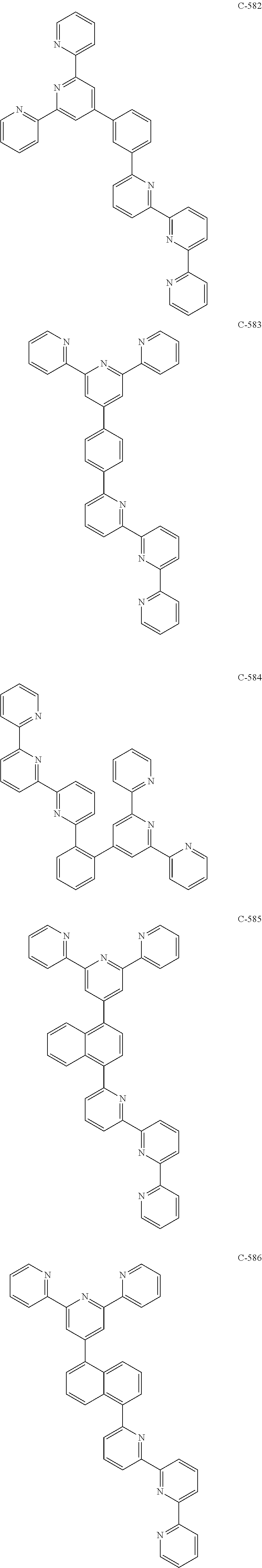 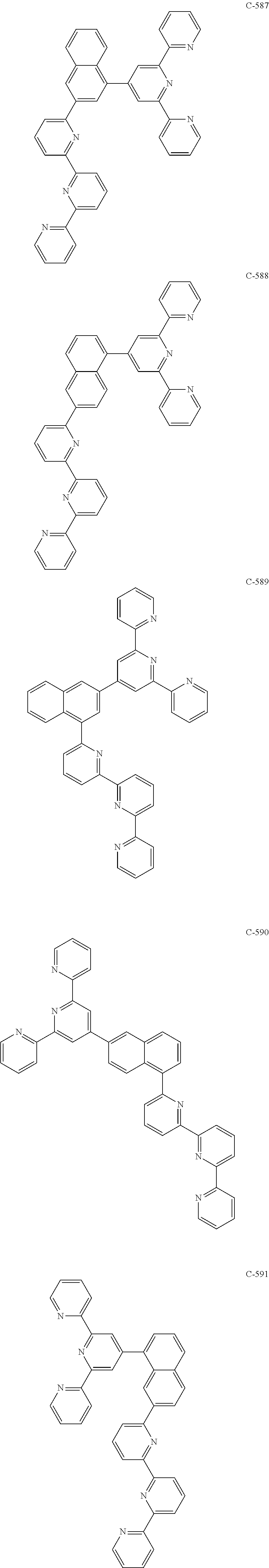 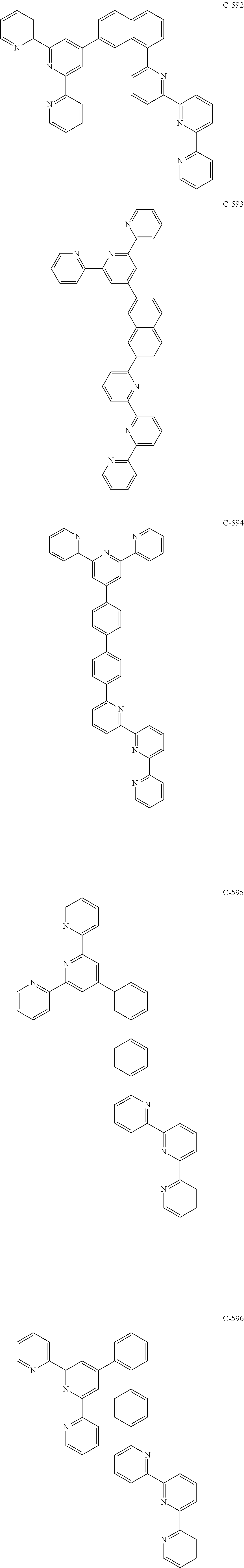 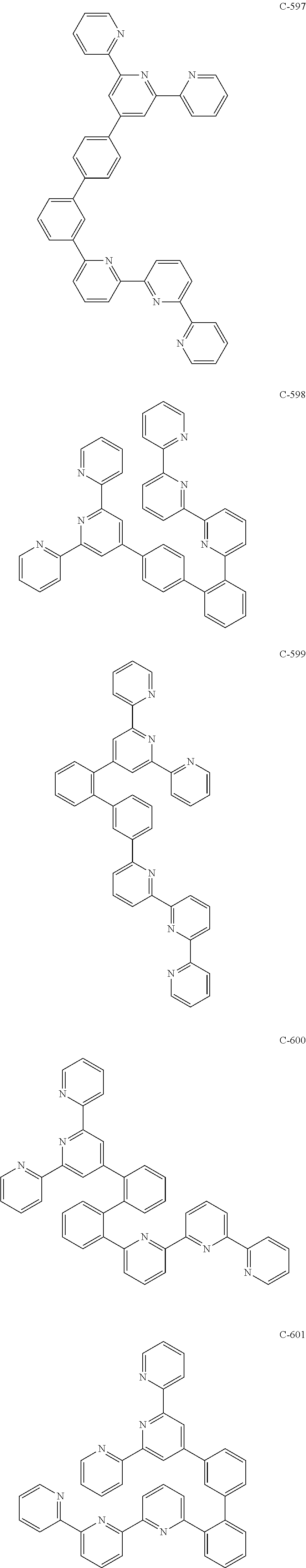 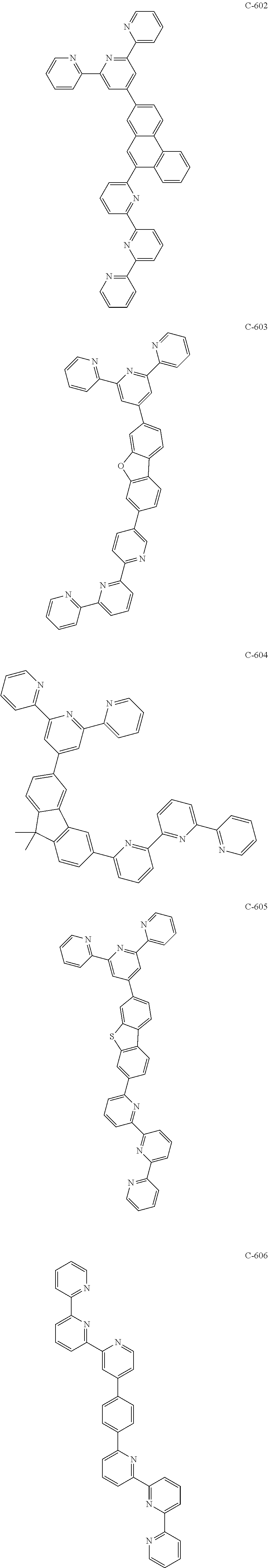 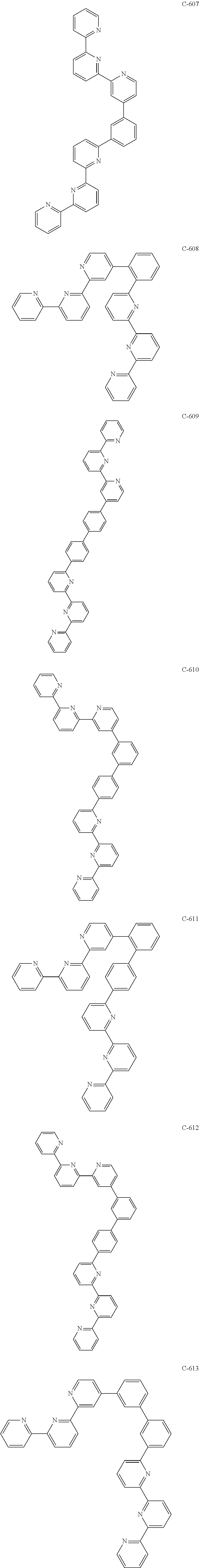 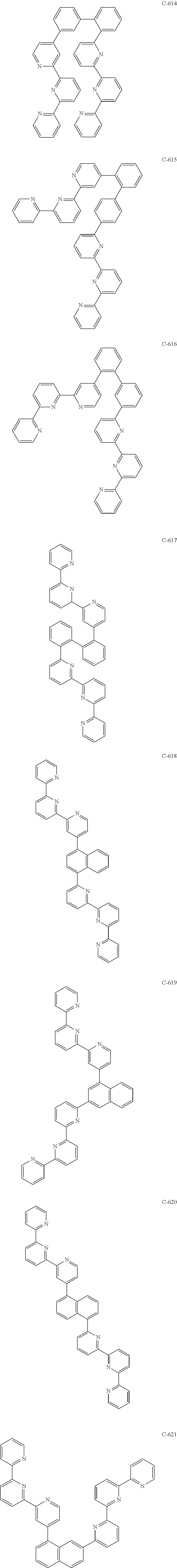 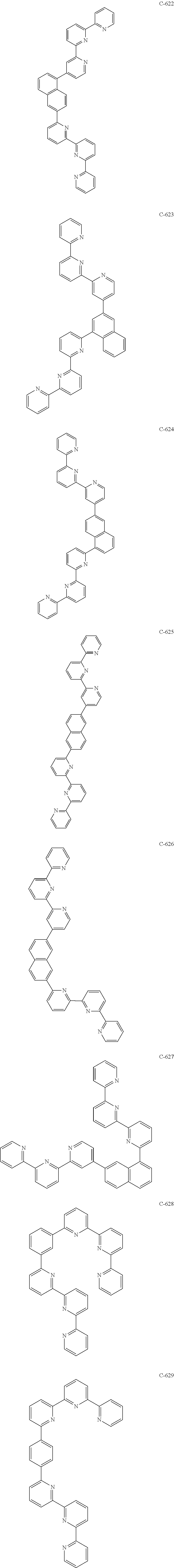 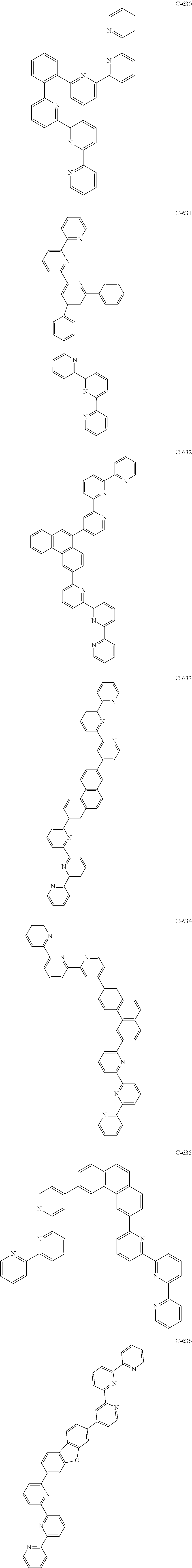 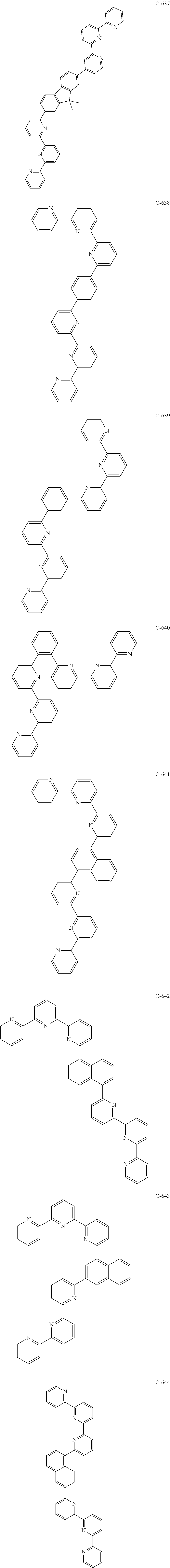 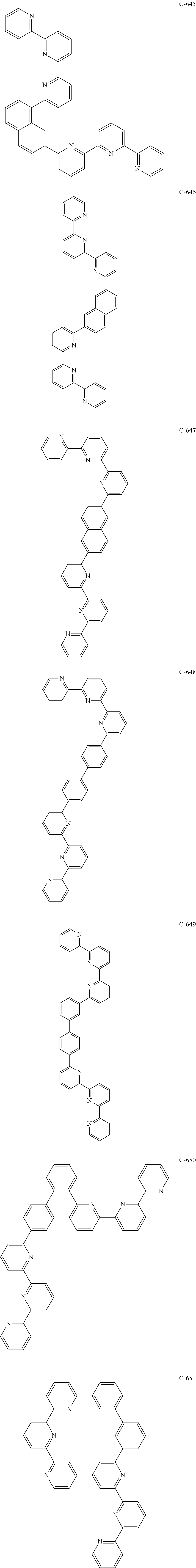 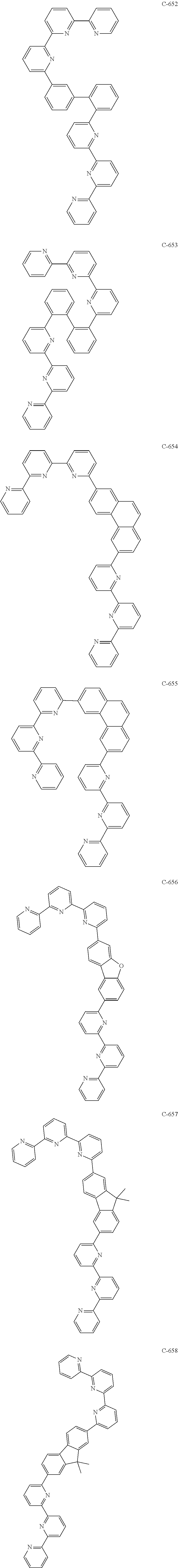 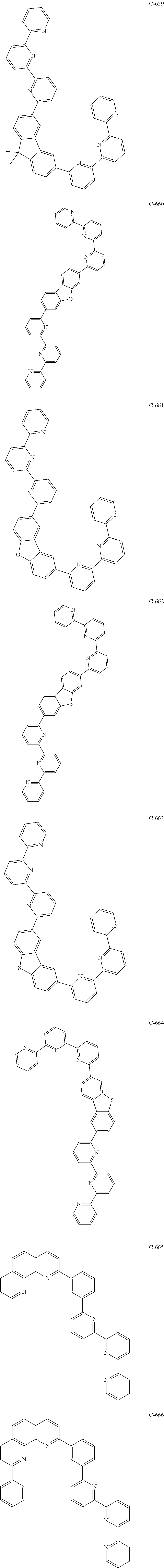 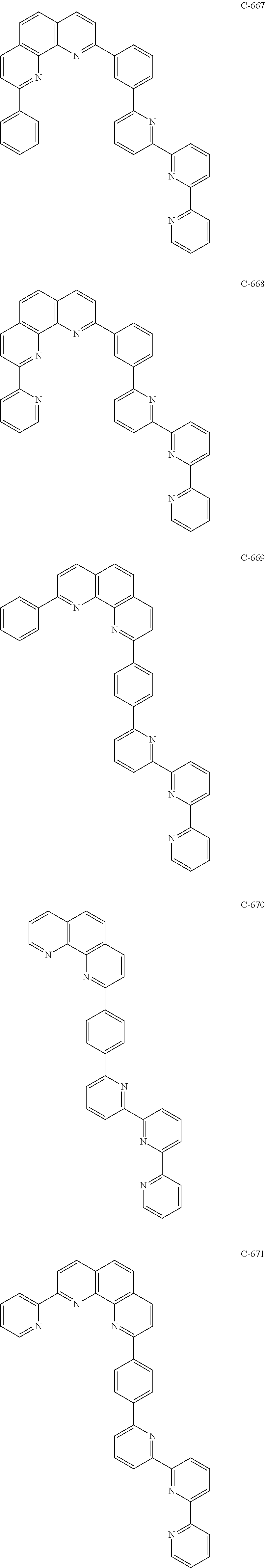 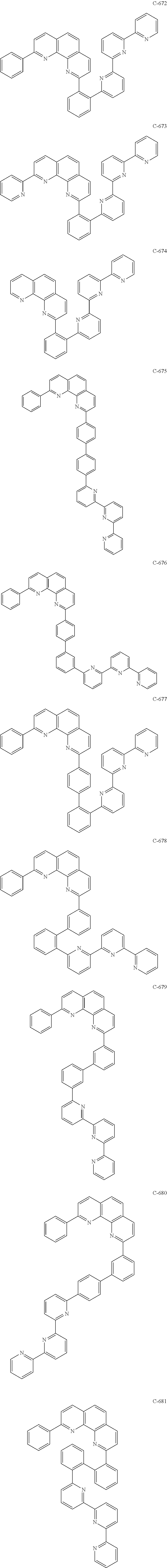 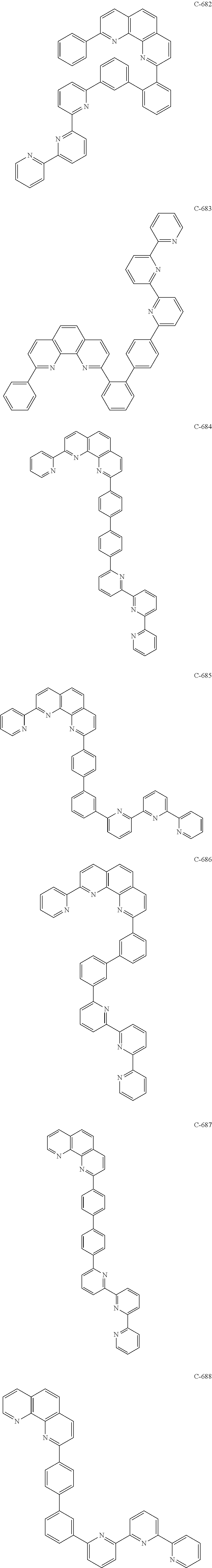 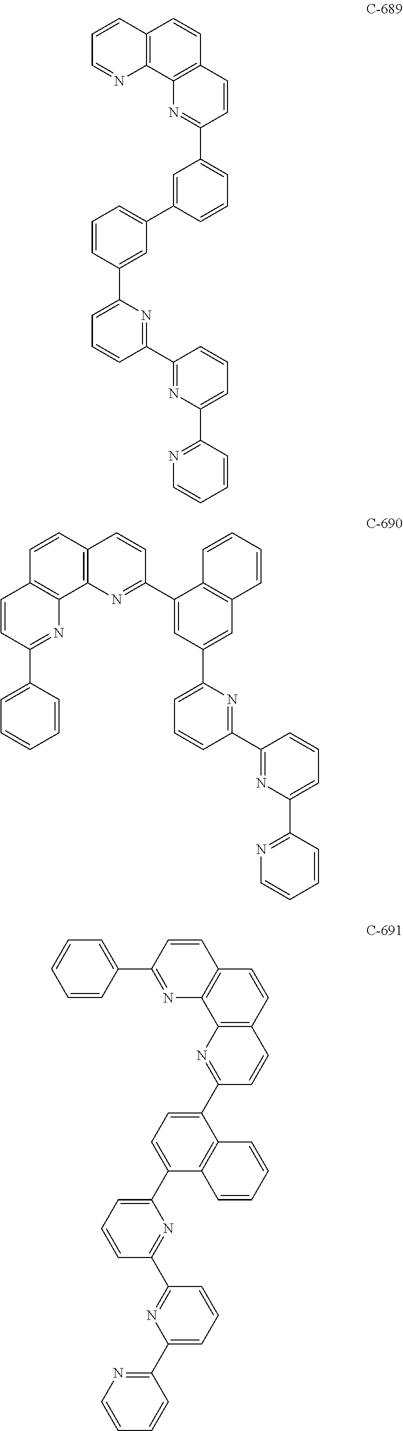 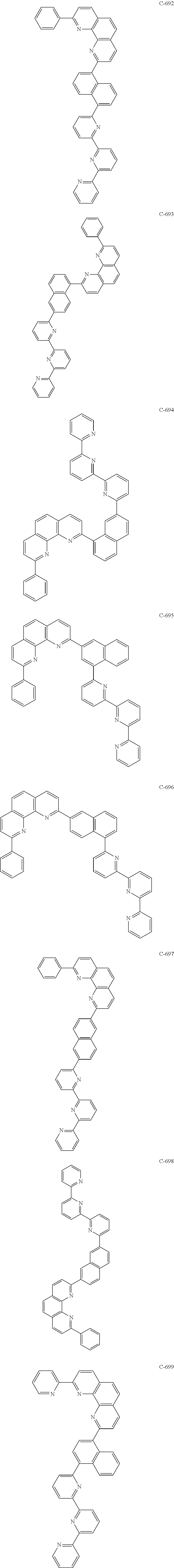 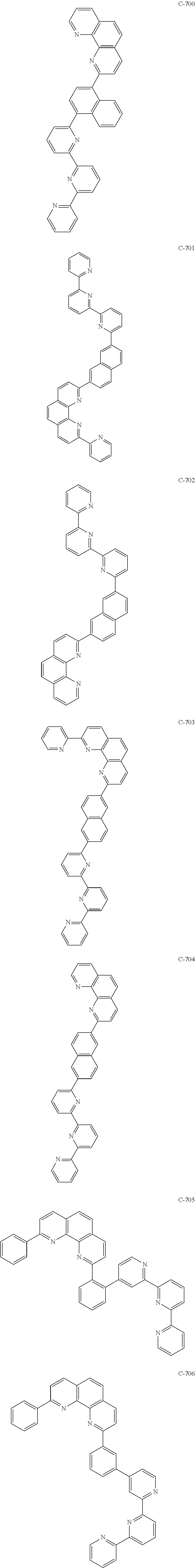 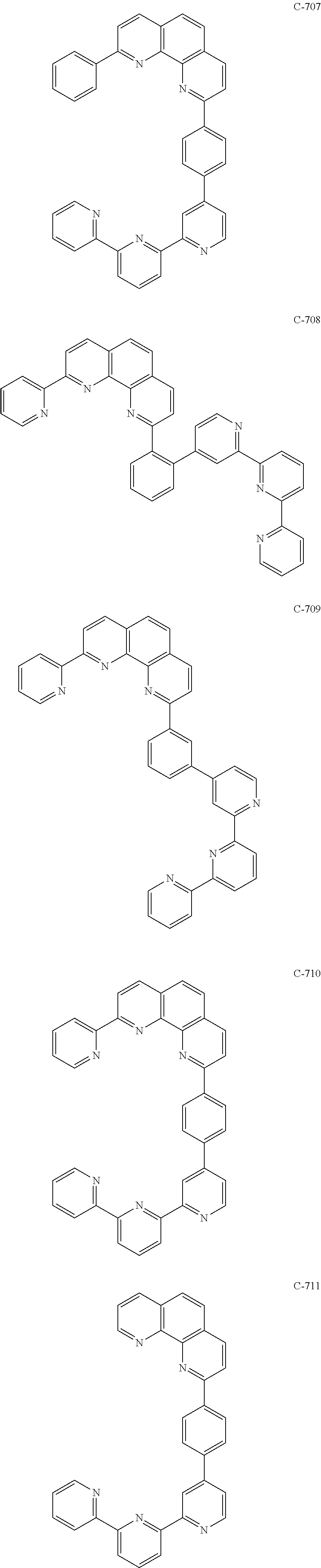 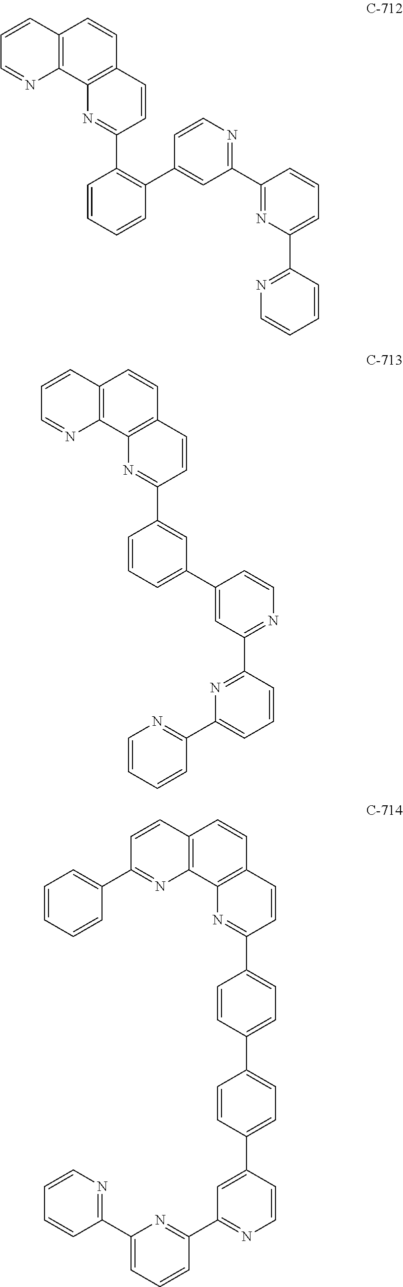 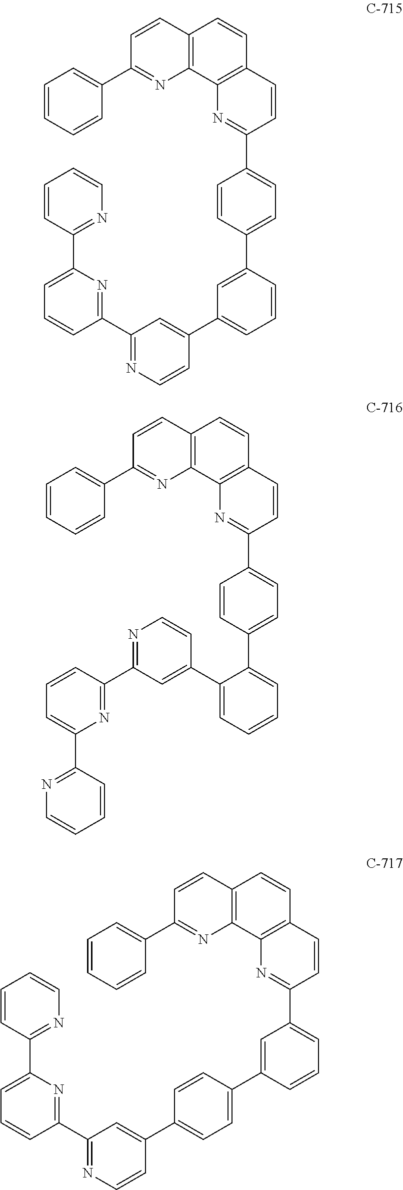 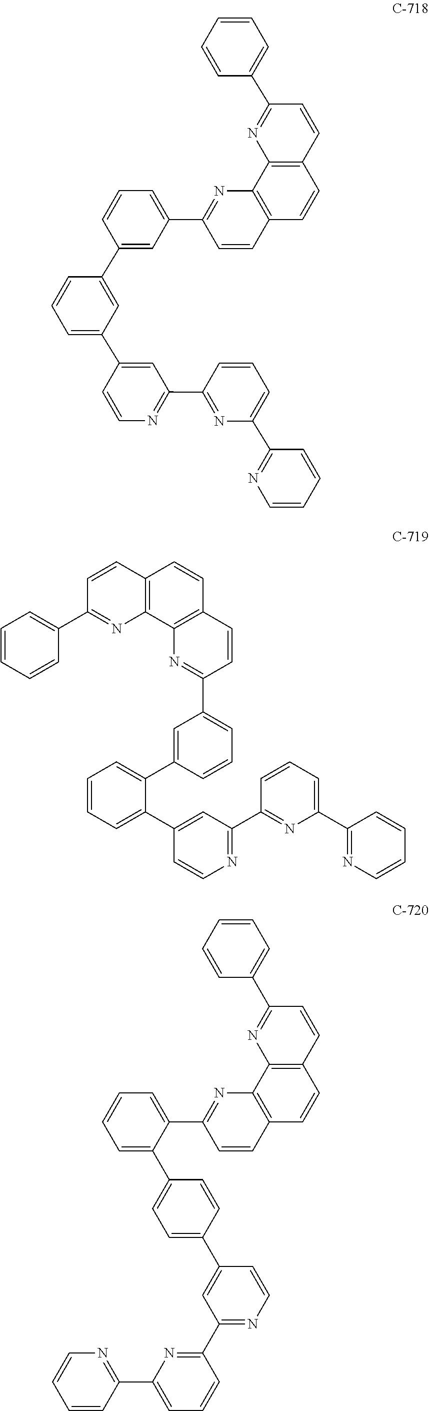 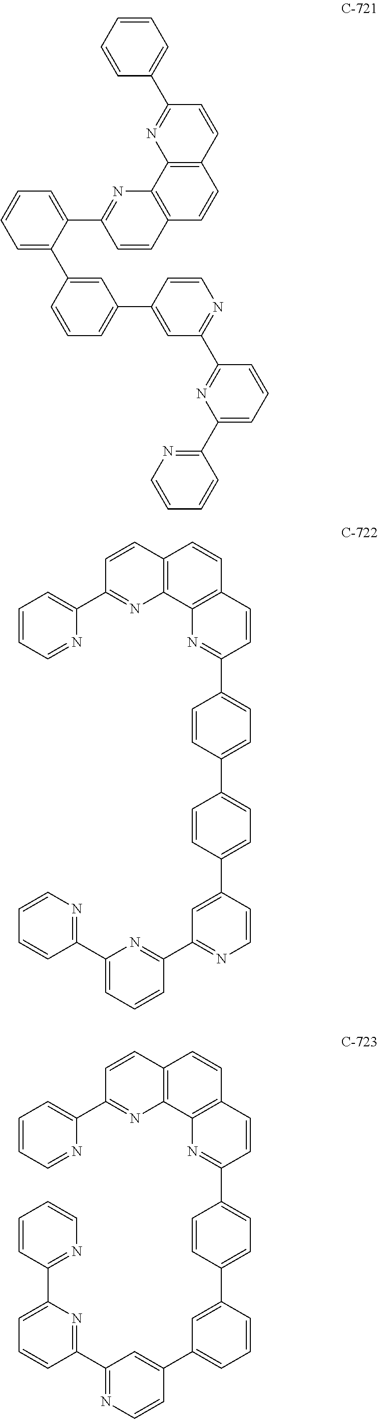 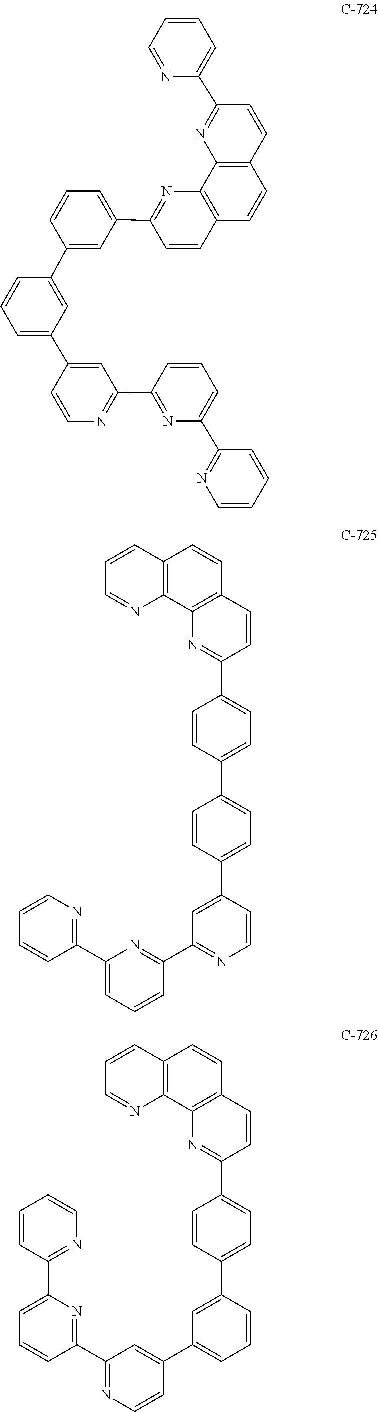 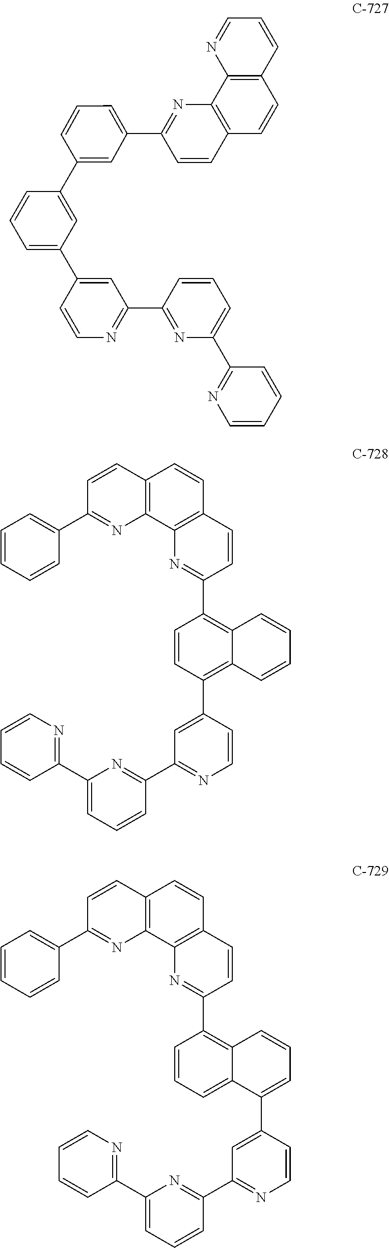 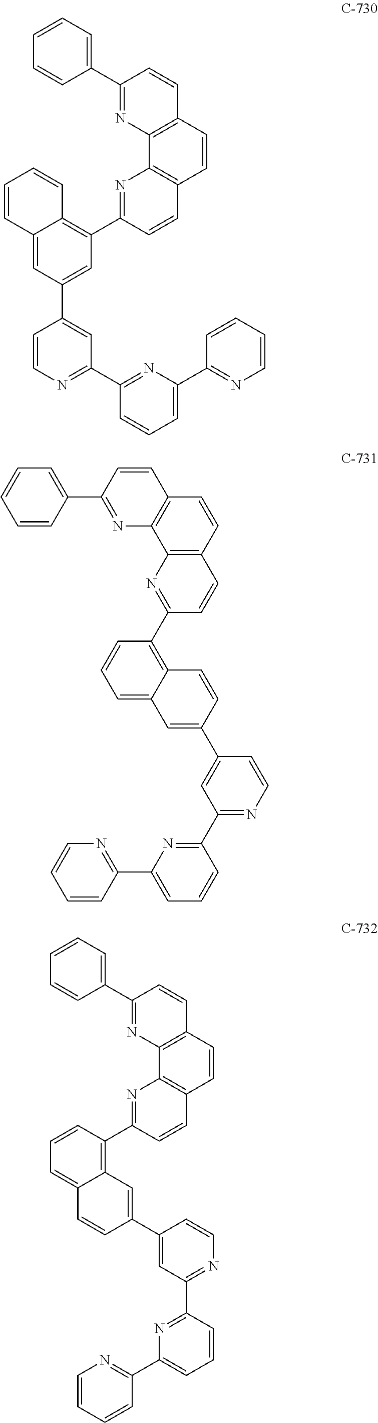 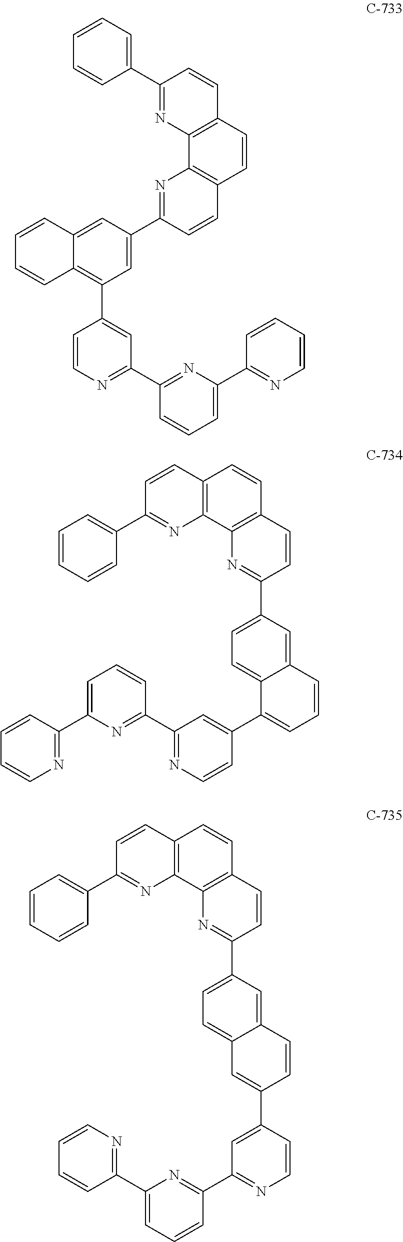 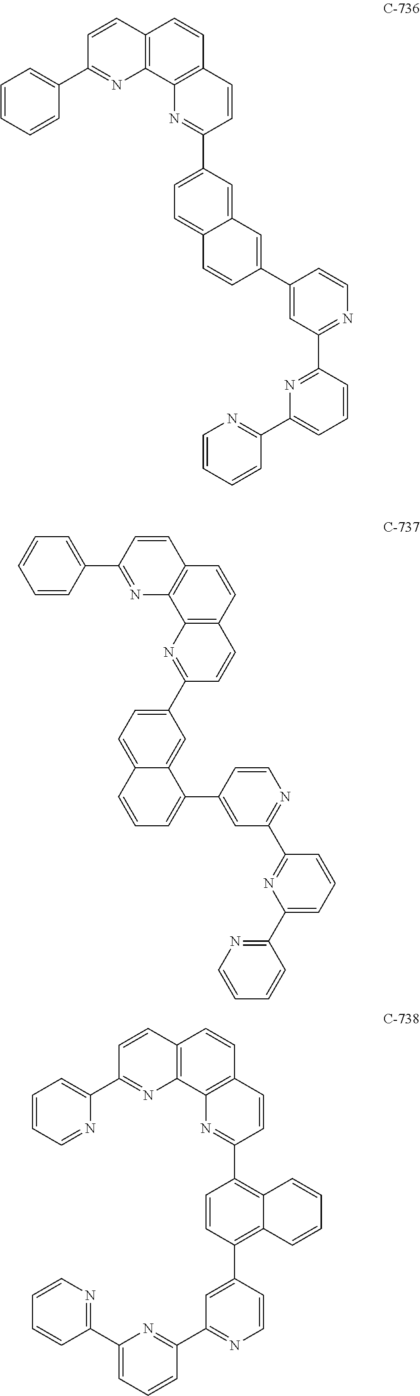 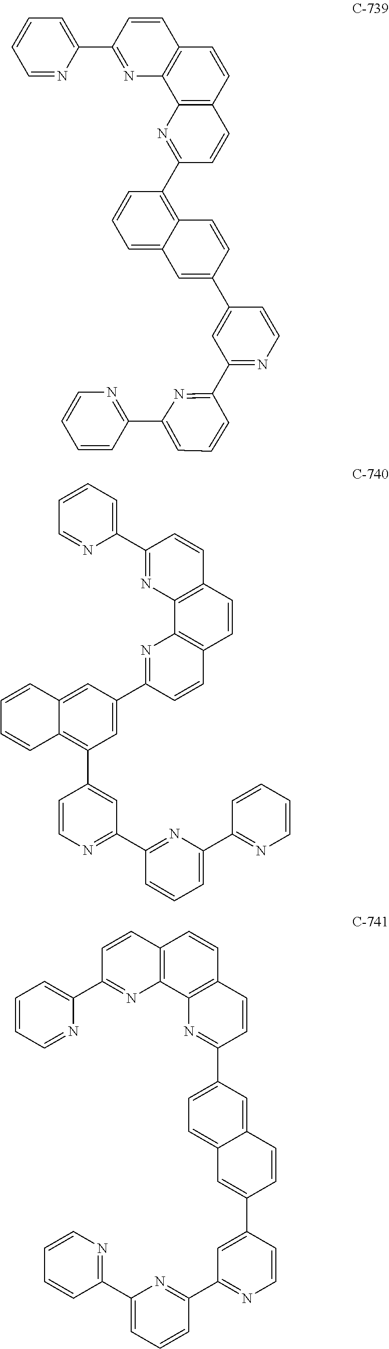 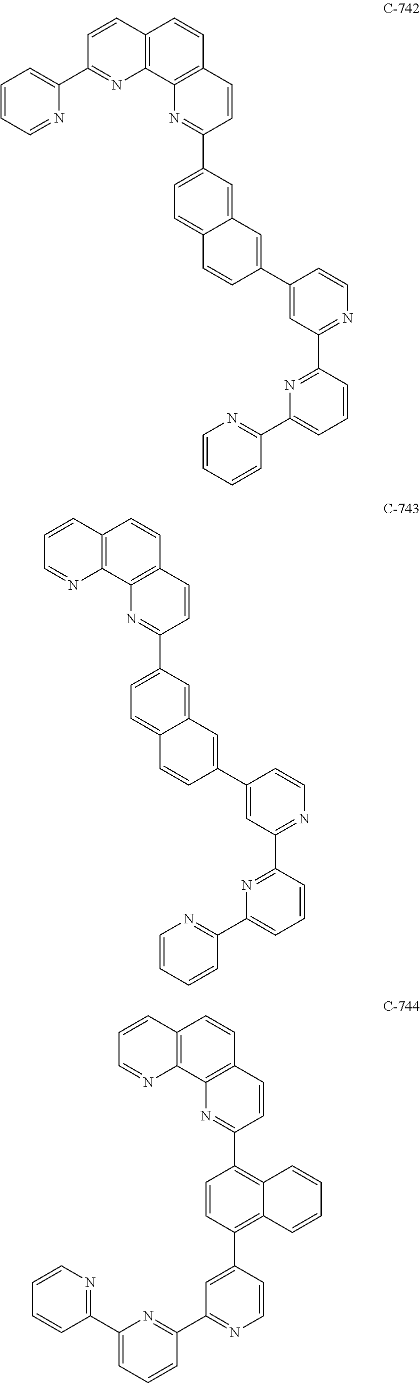 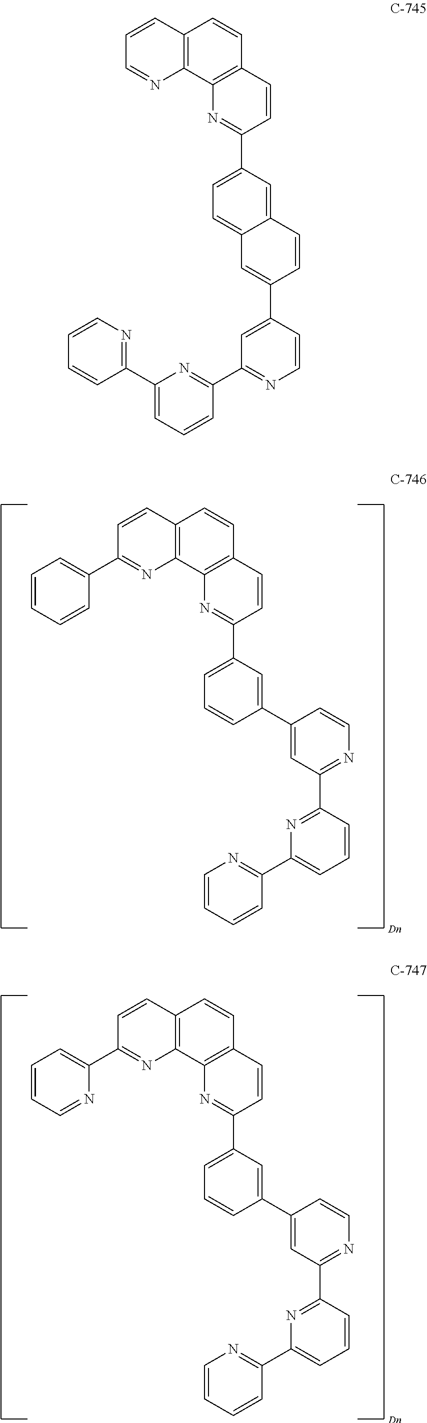 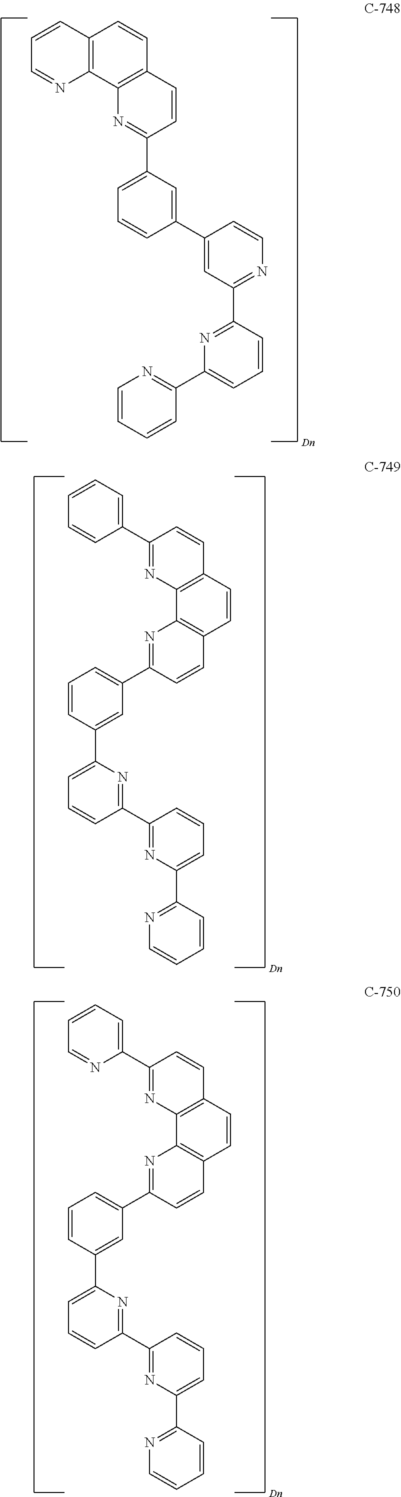 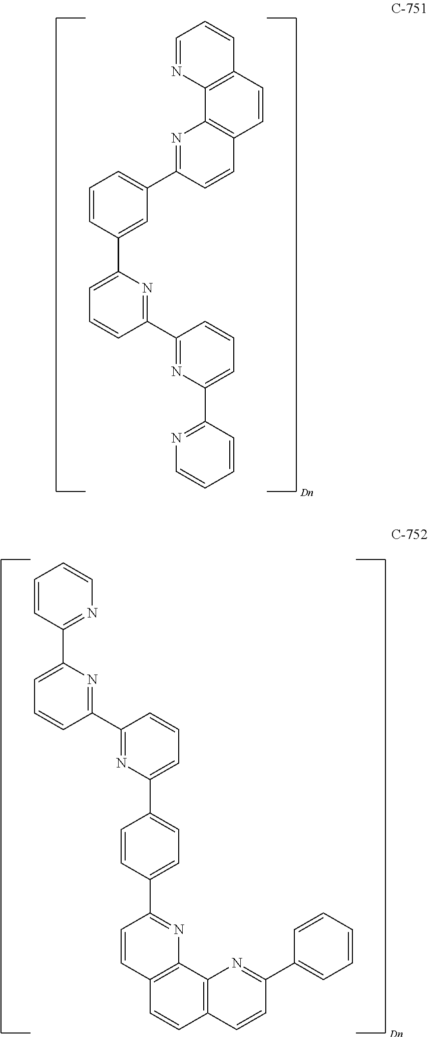 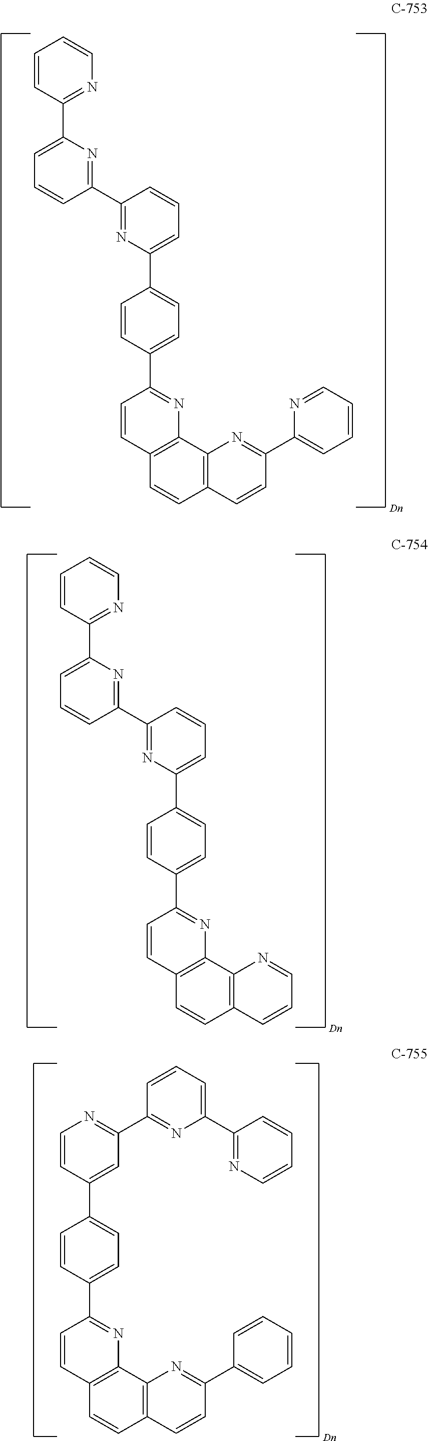 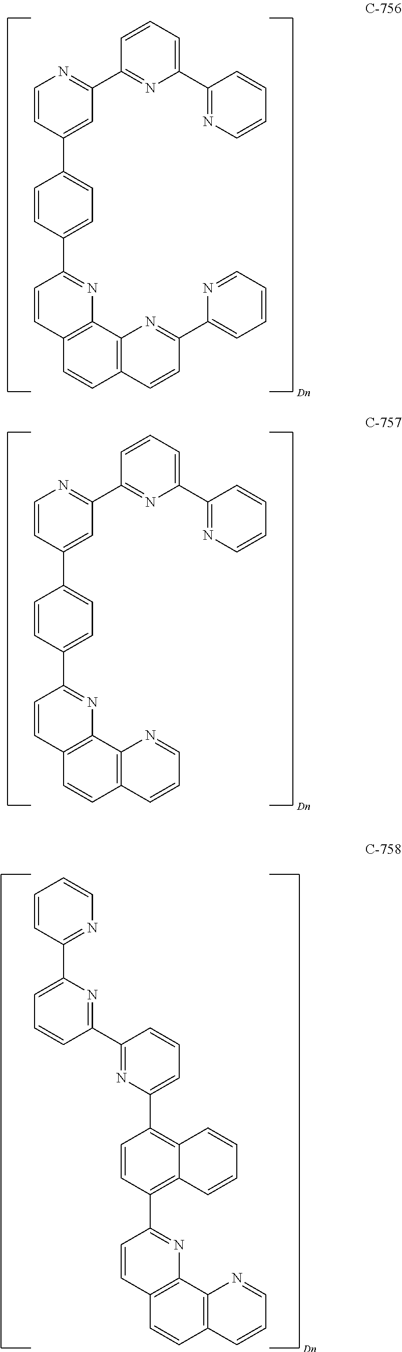 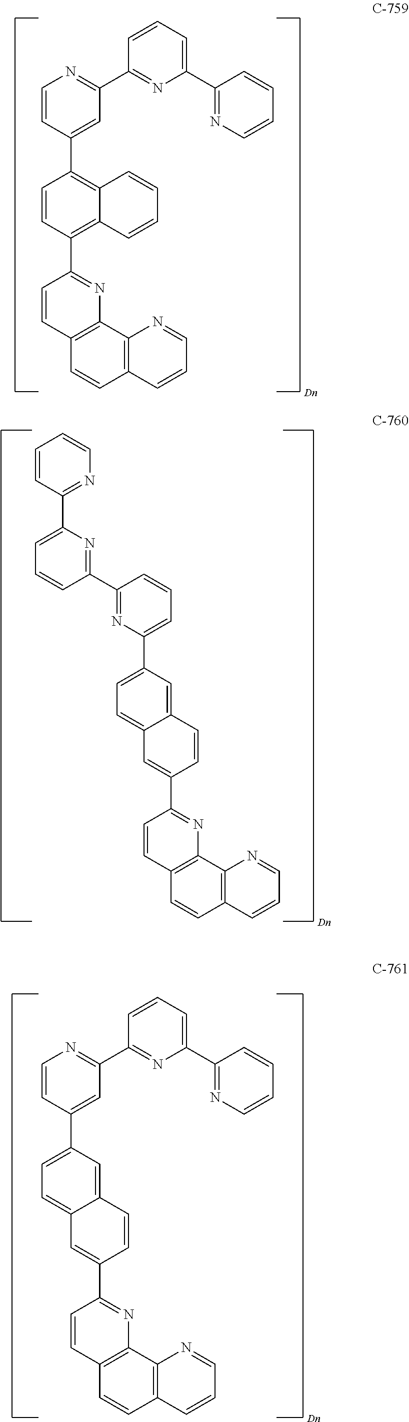 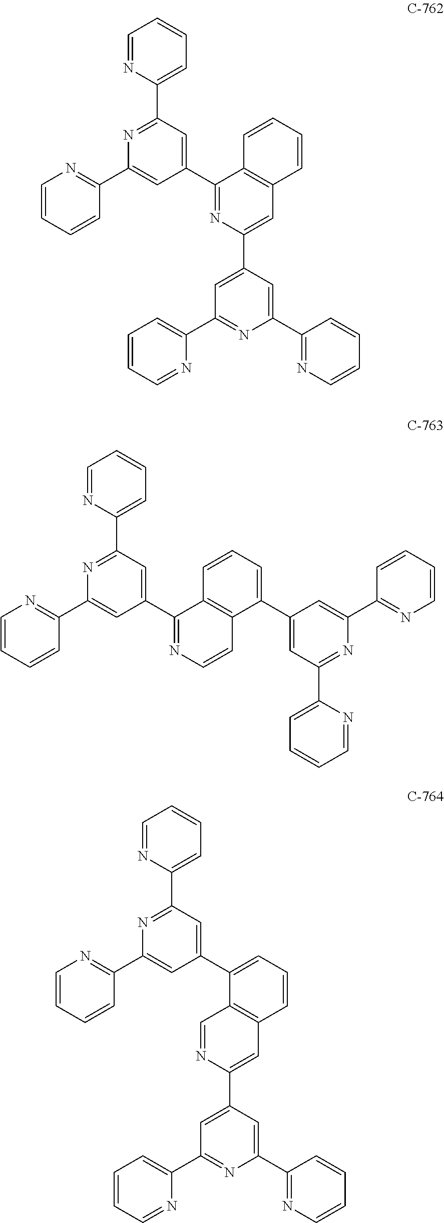 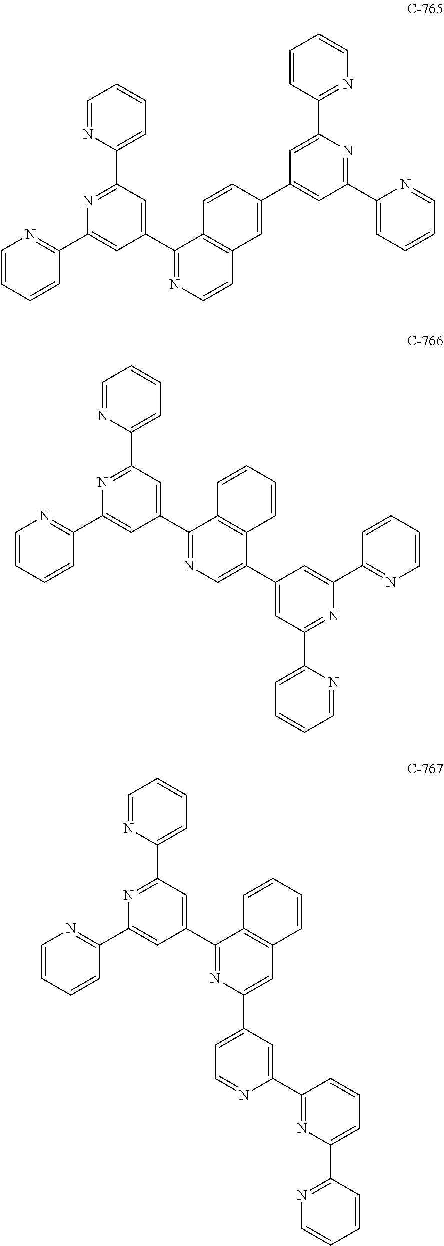 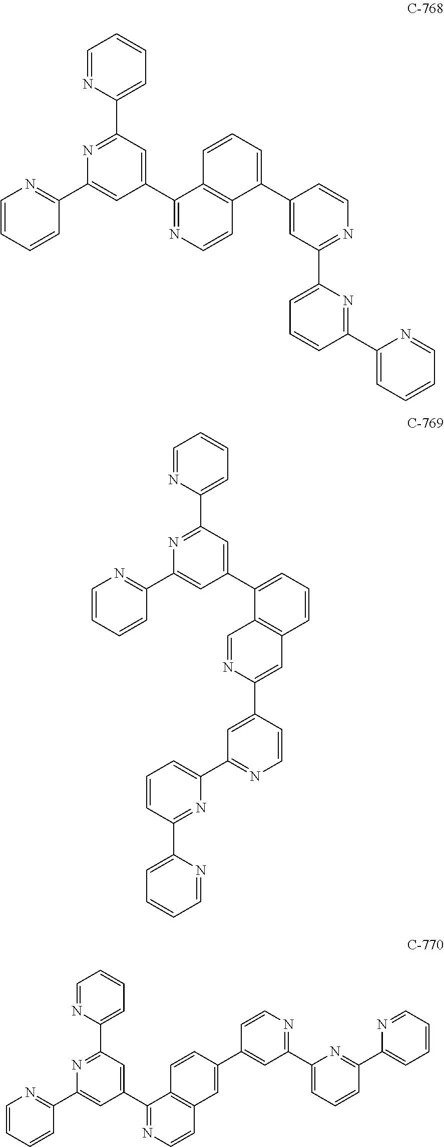 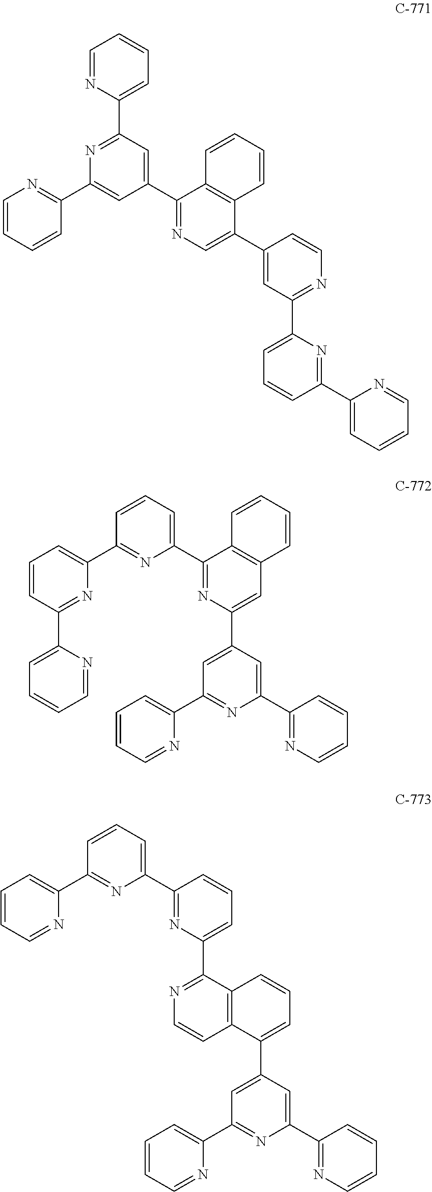 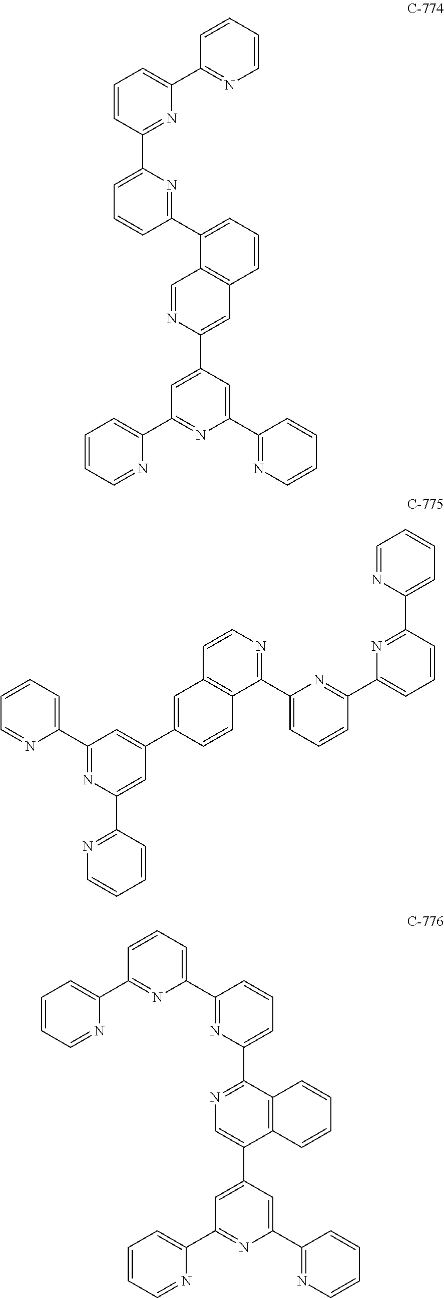 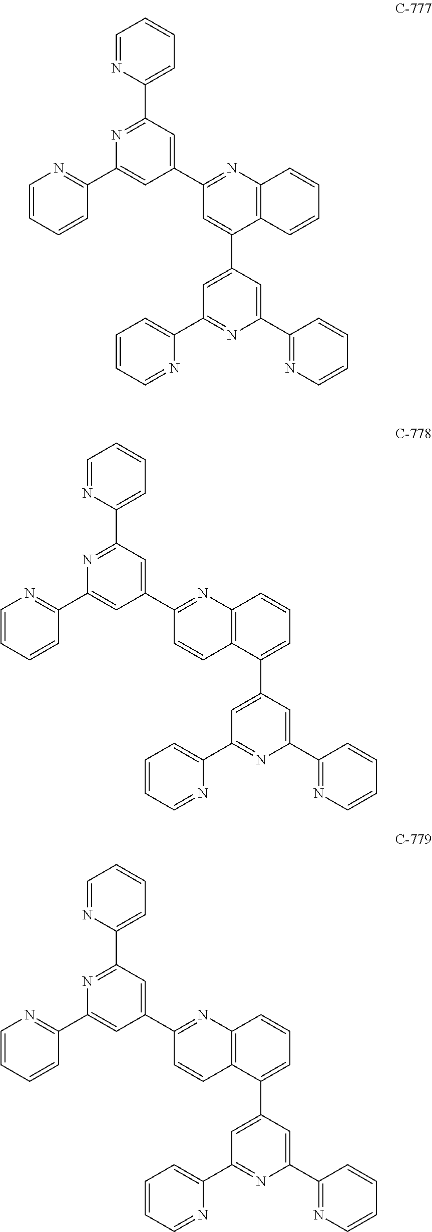 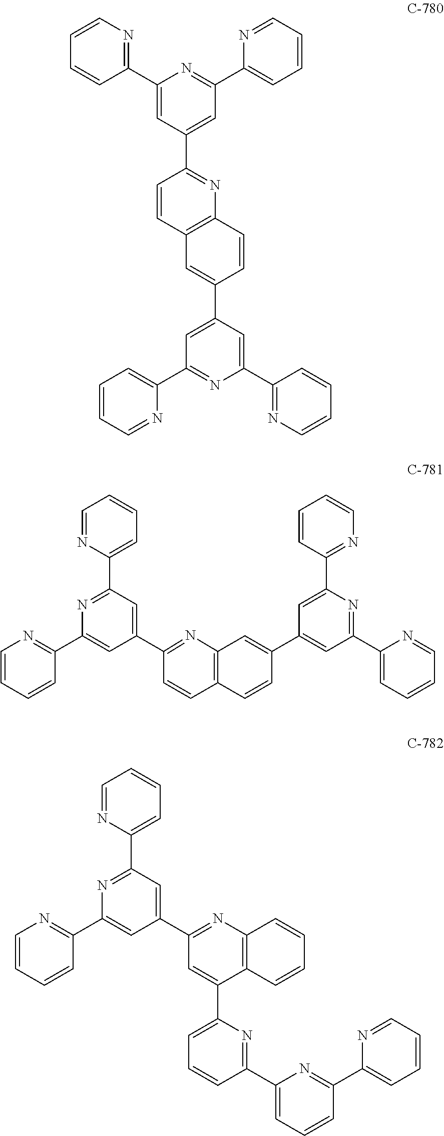 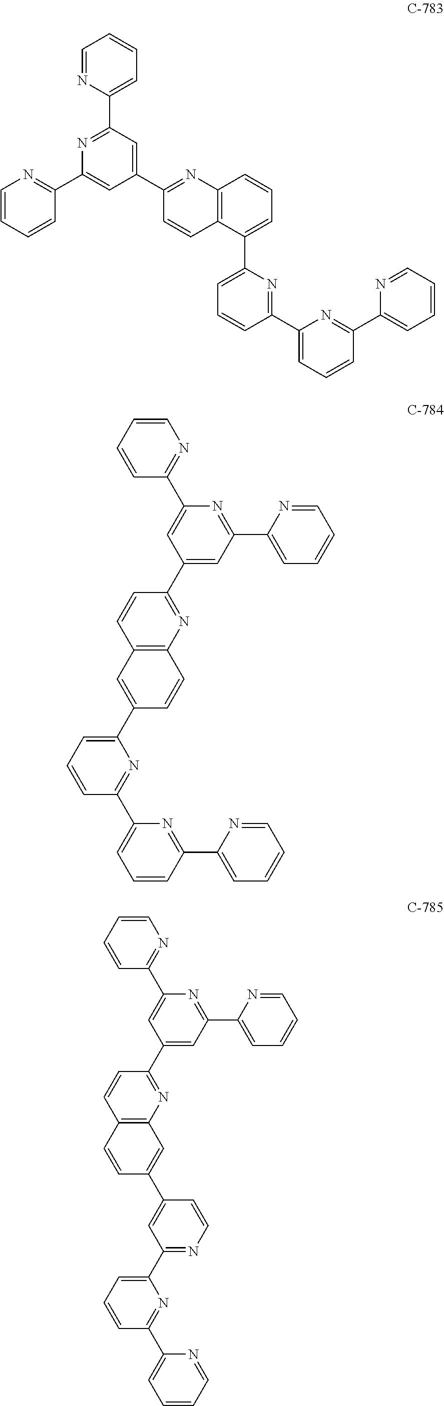 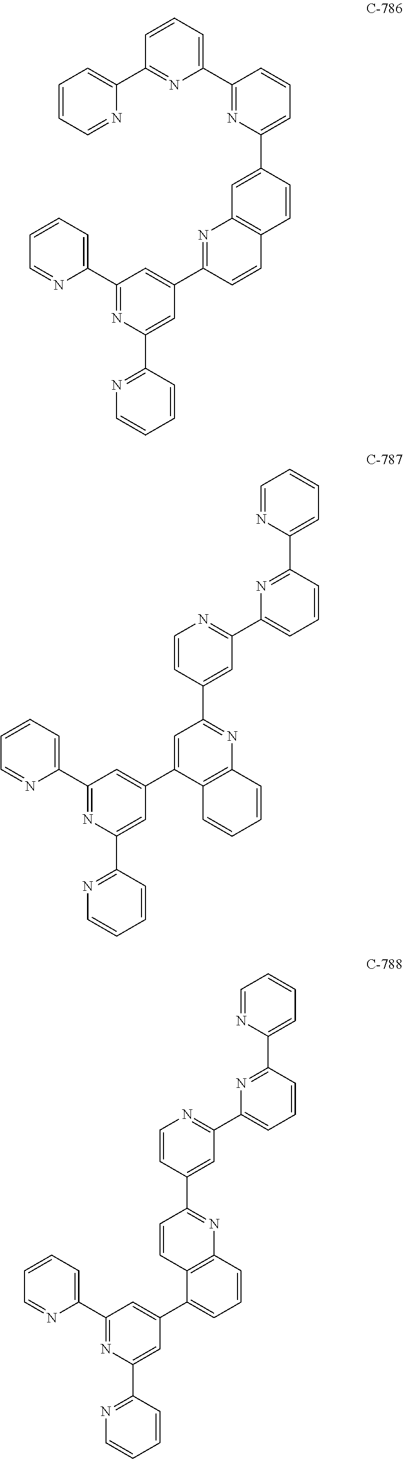 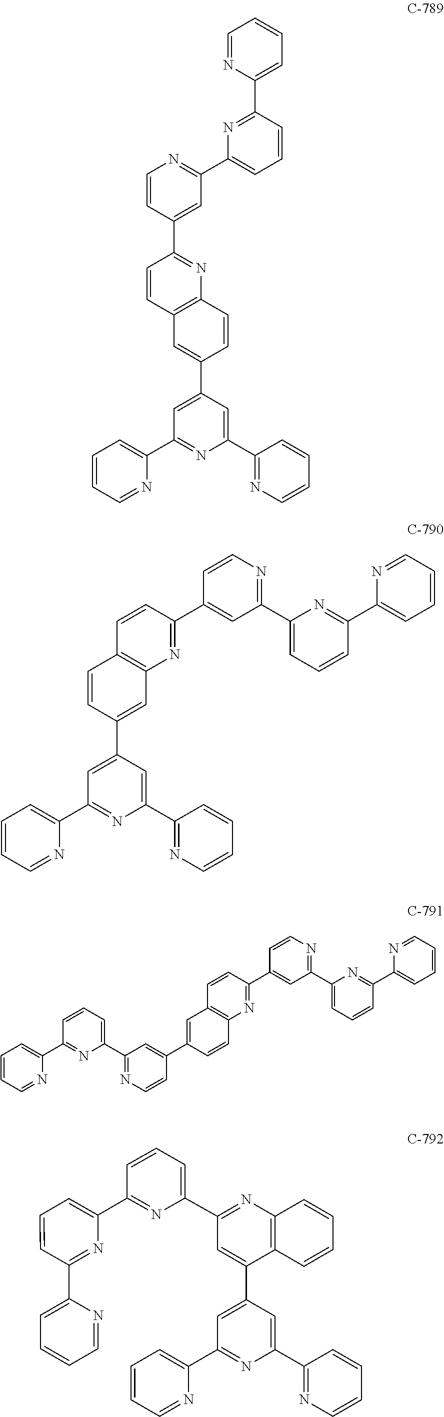 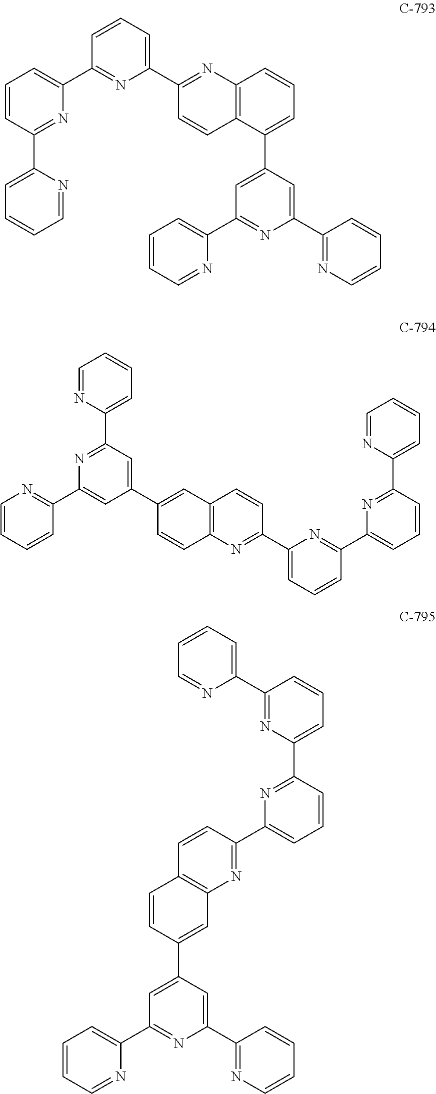 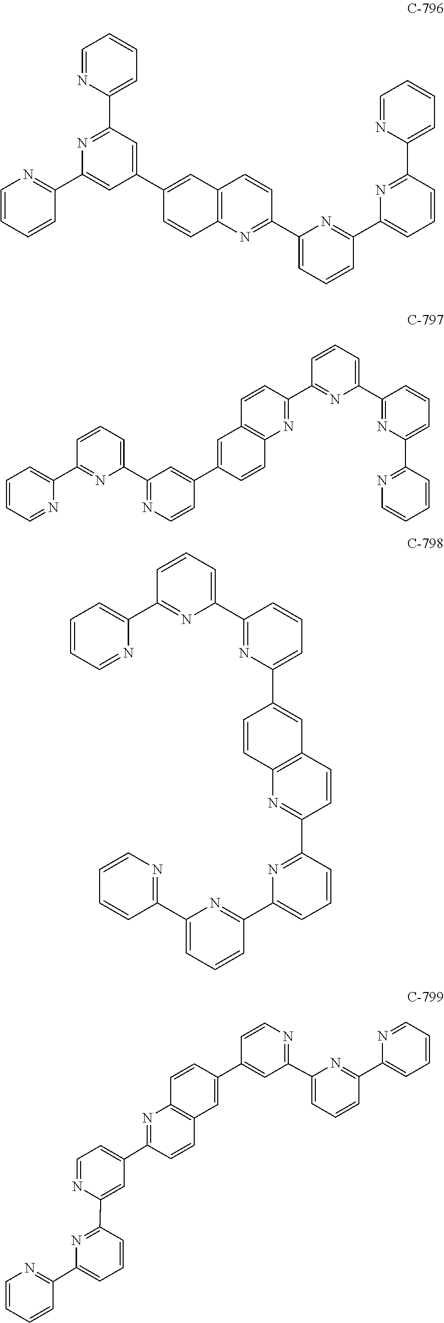 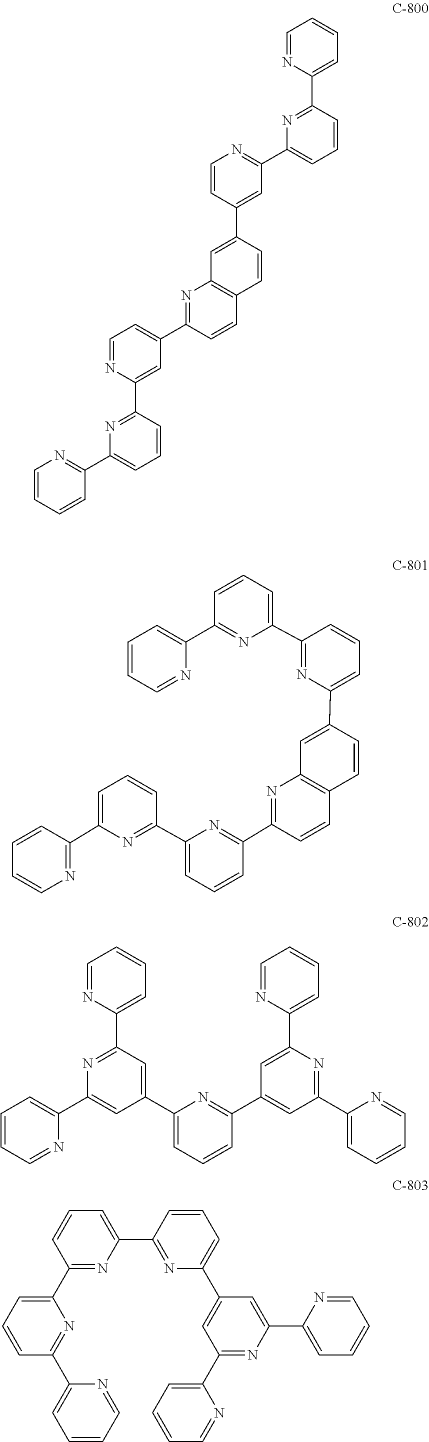 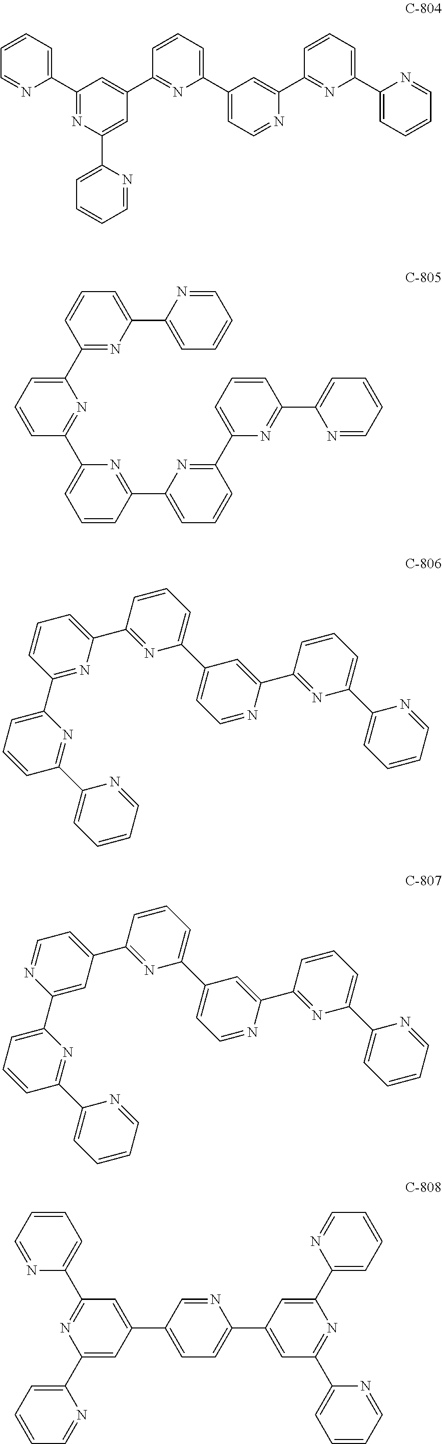 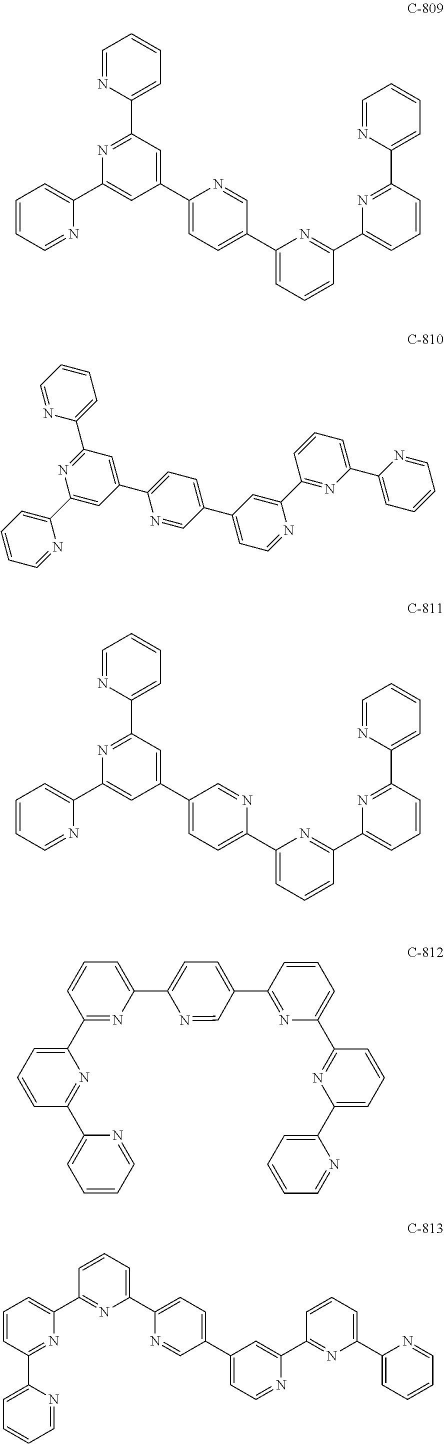 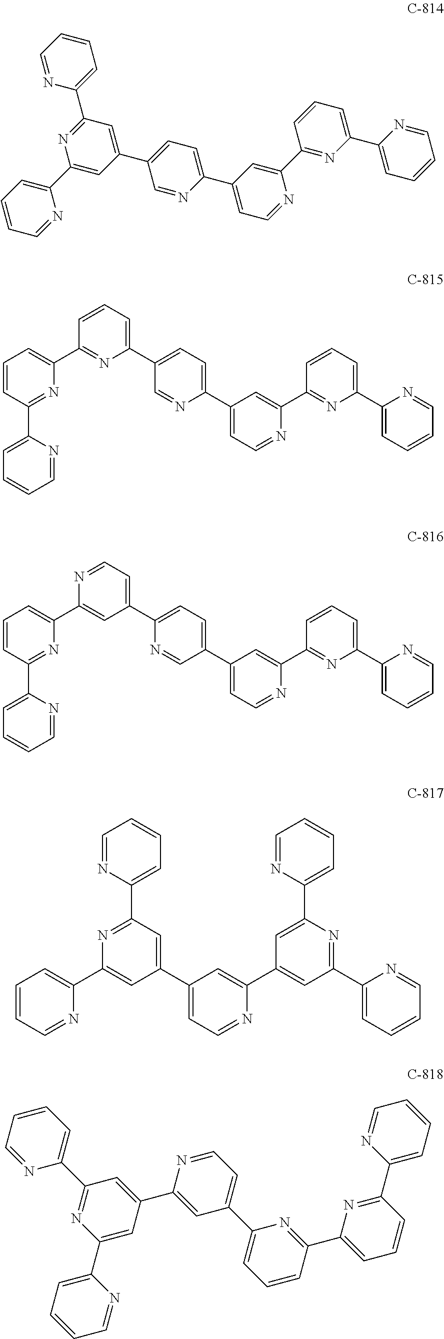 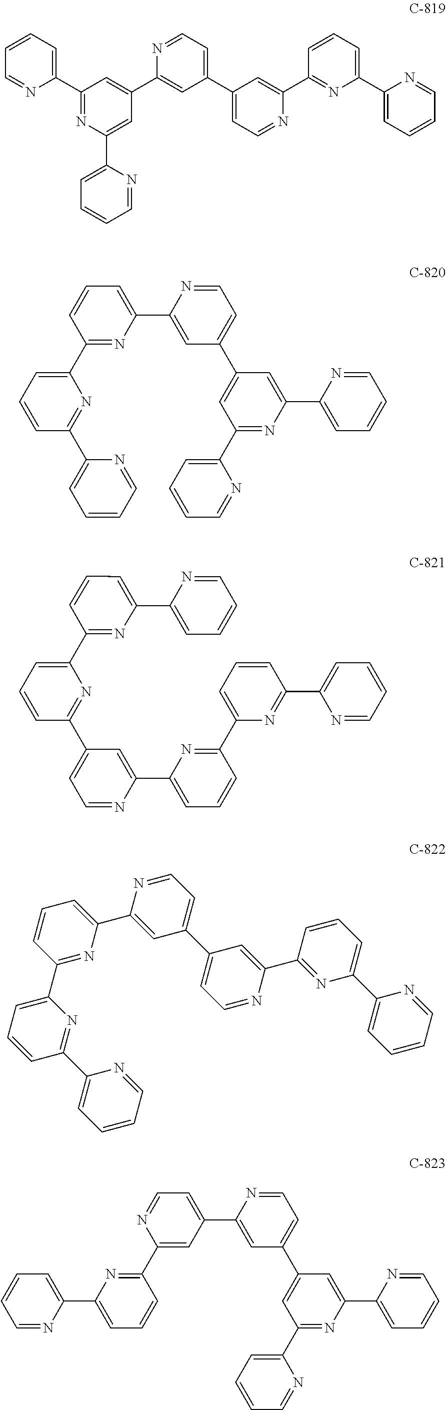 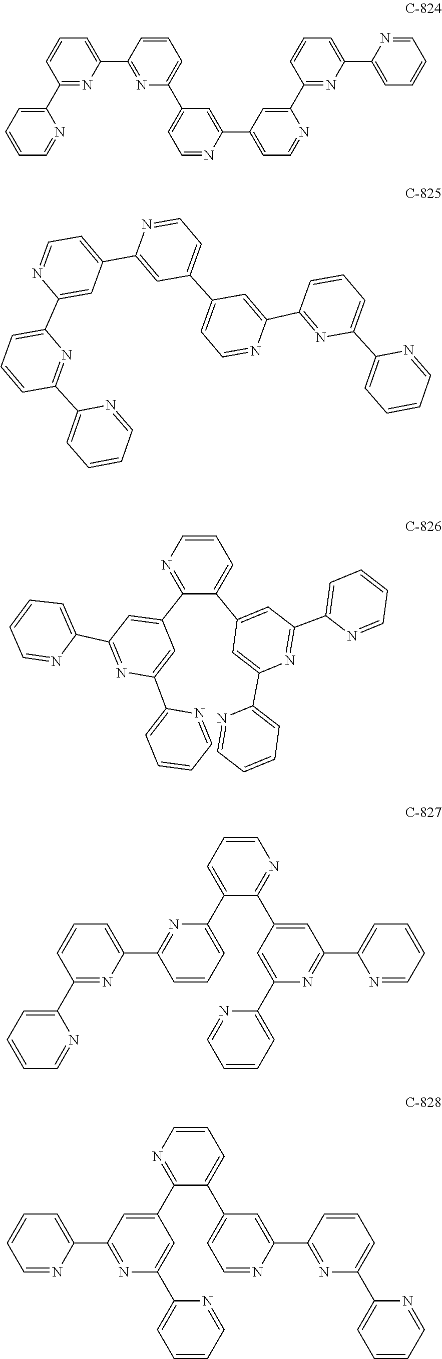 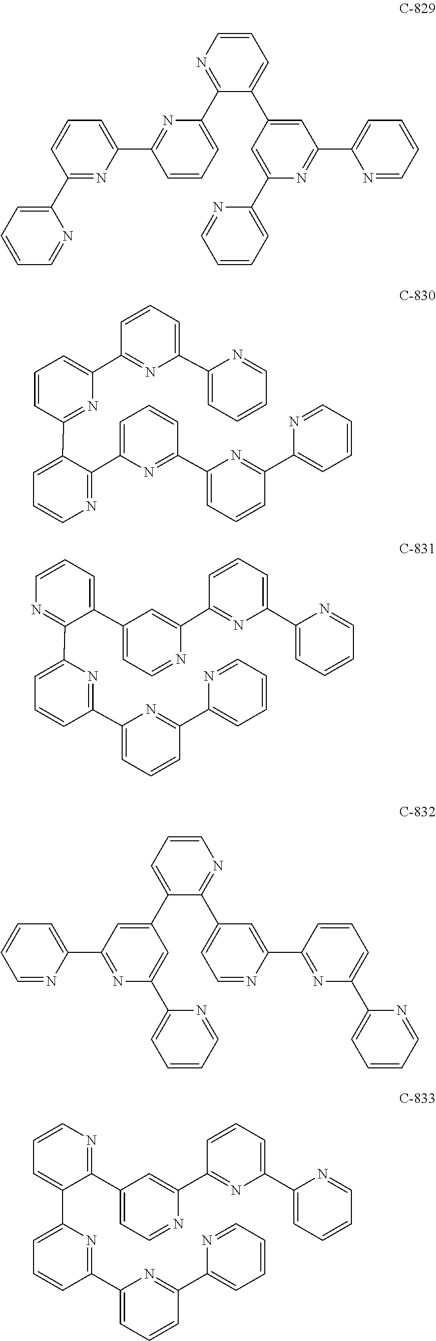 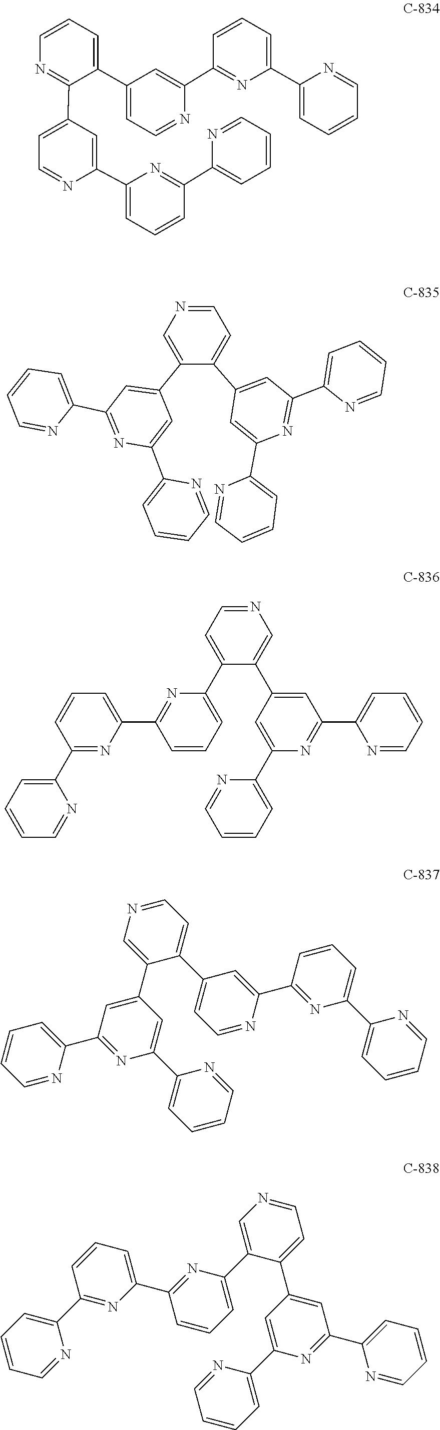 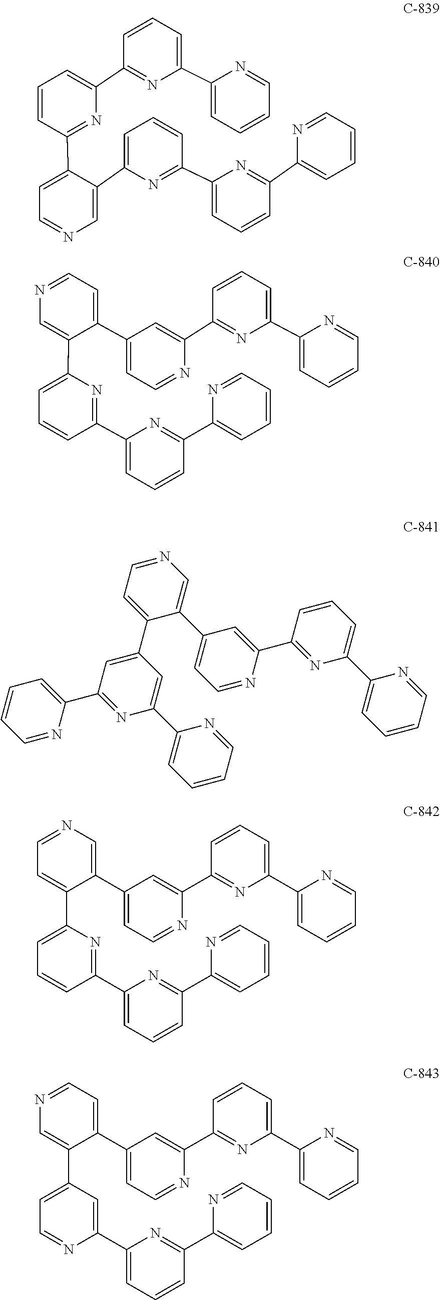 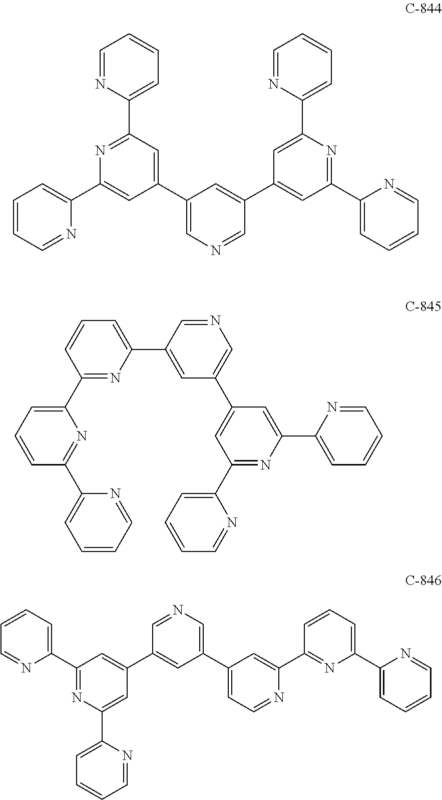 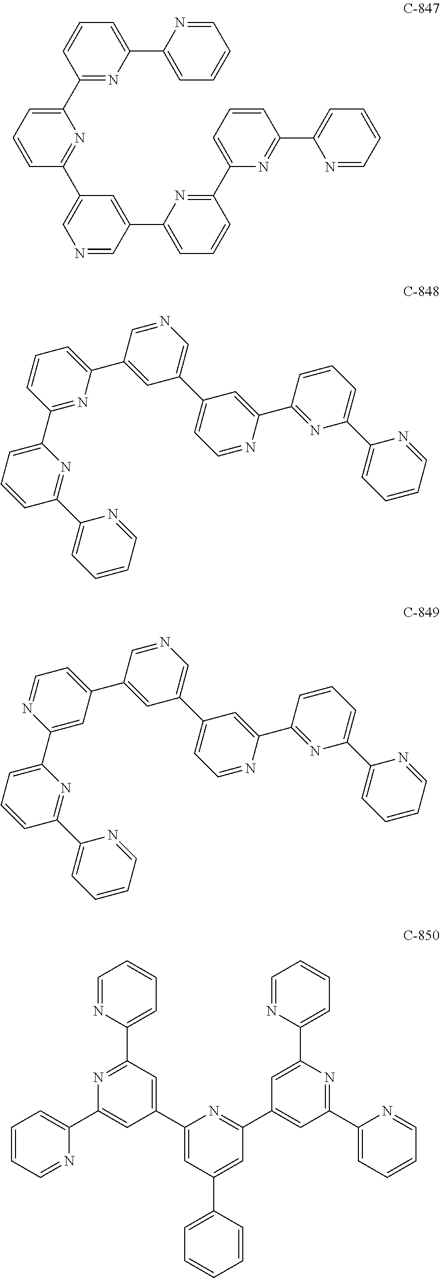 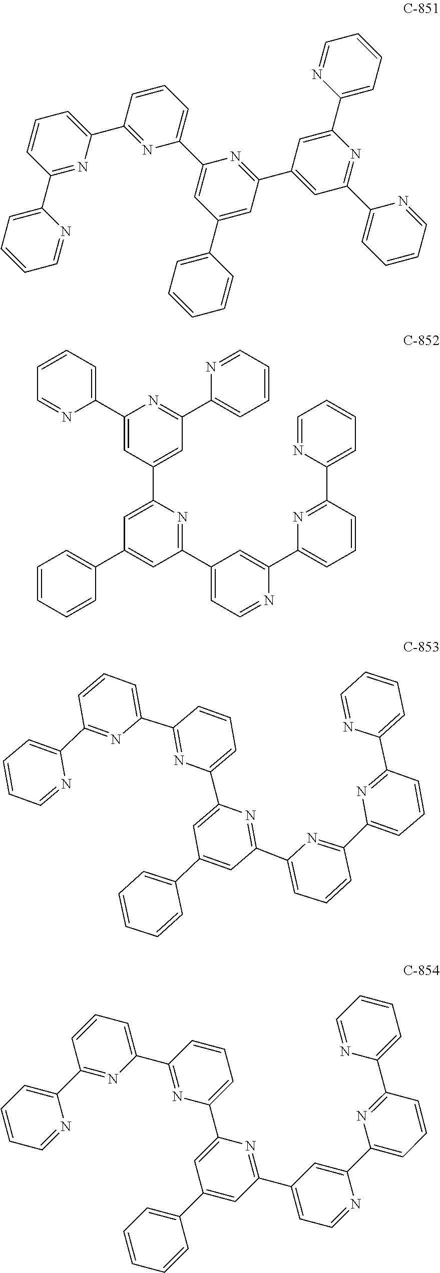 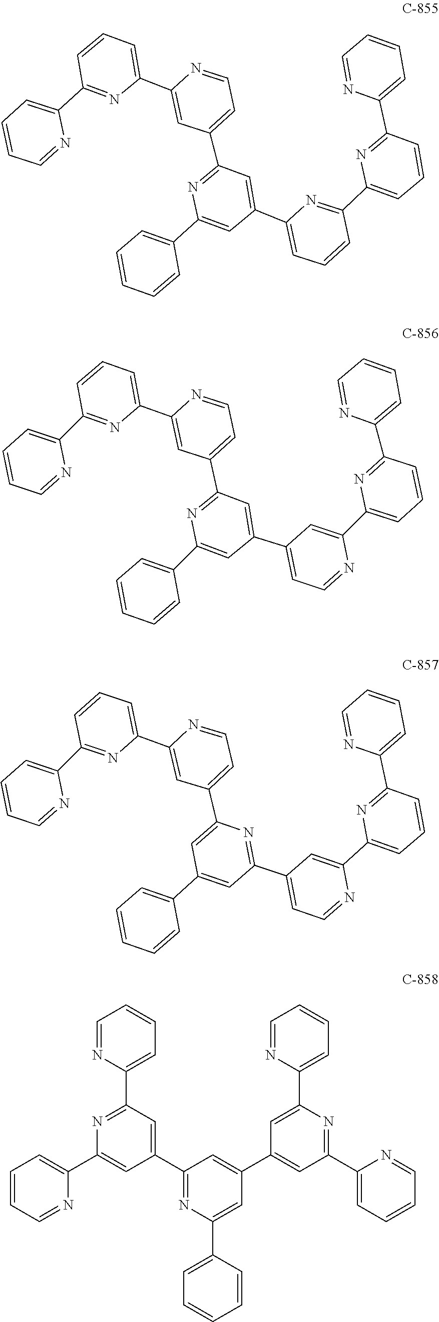 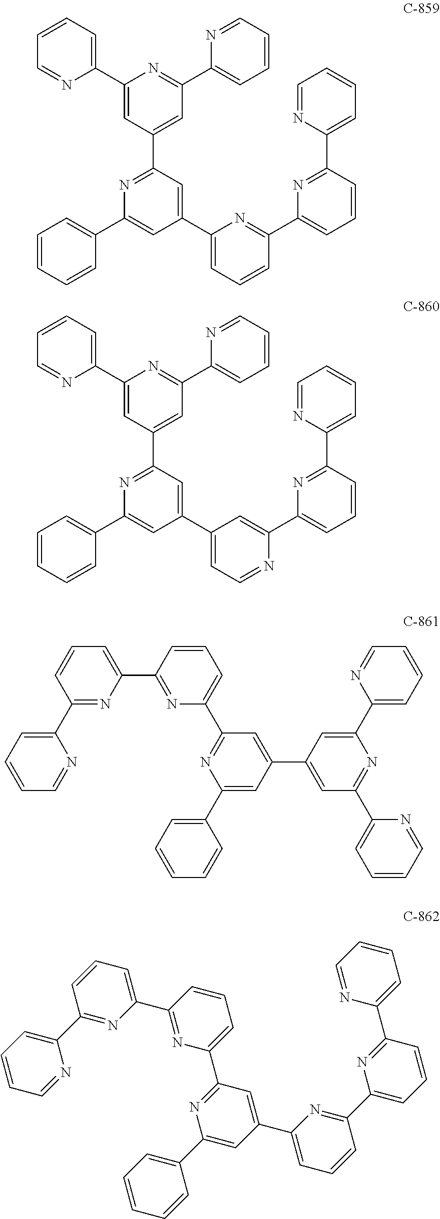 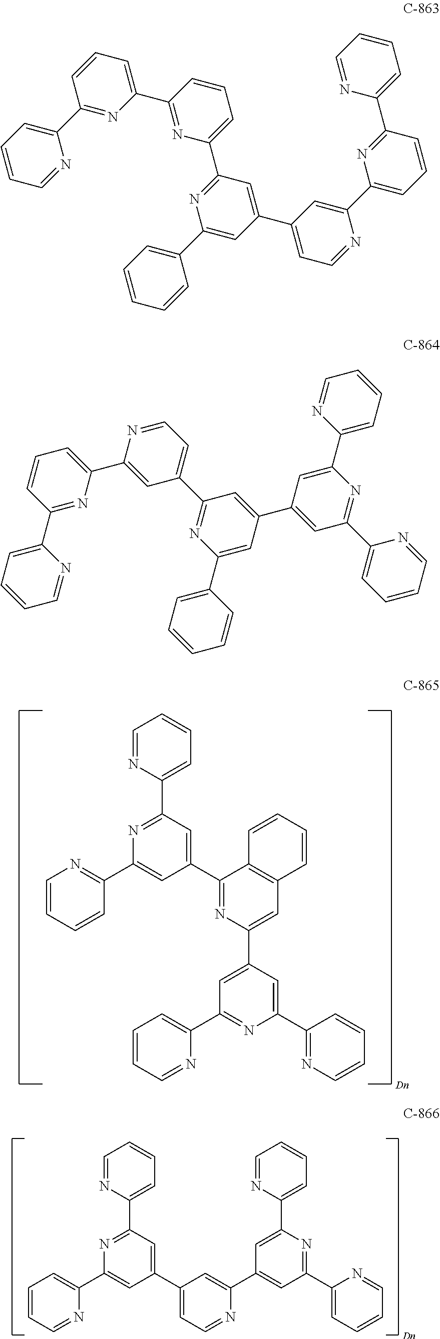 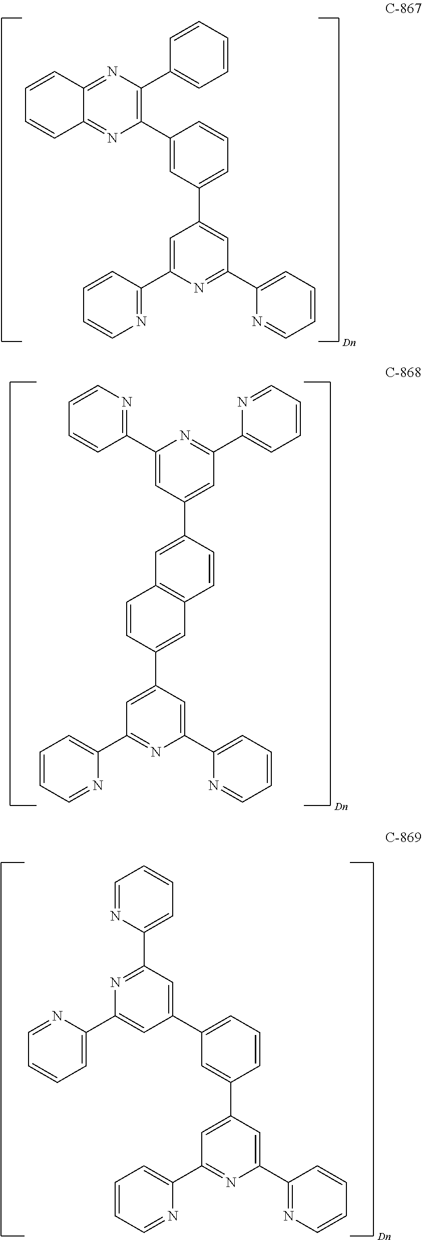 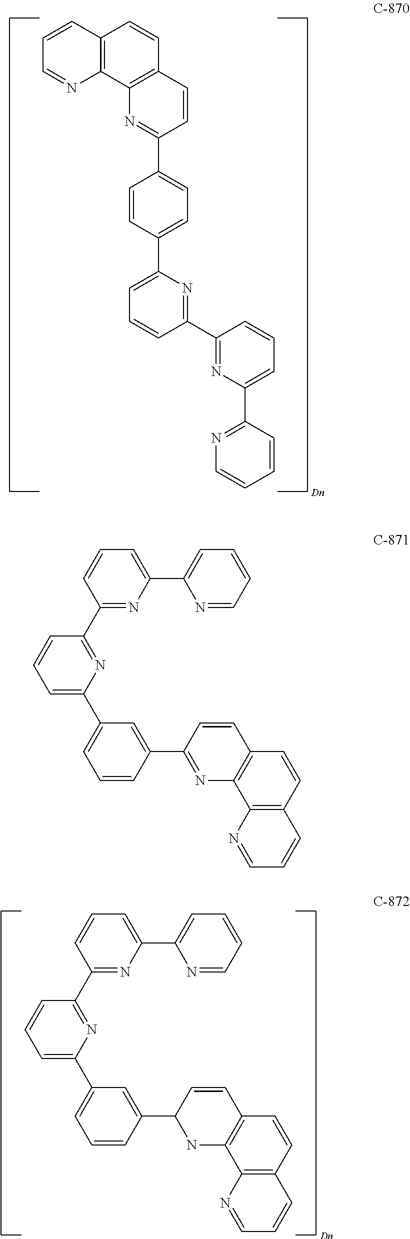 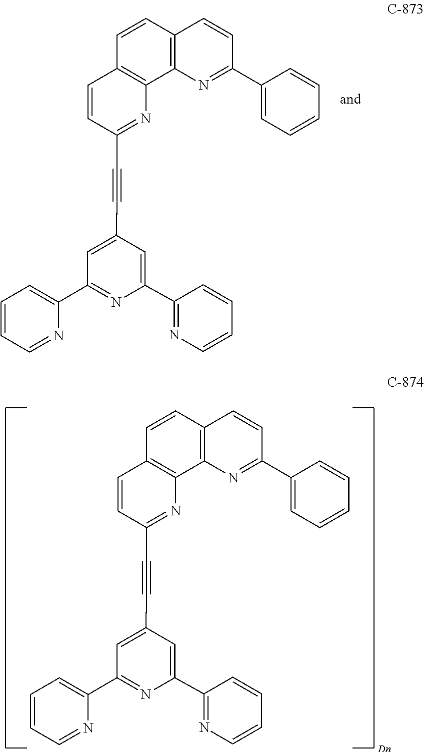
wherein Dₙ means that n hydrogens are replaced by deuterium, n is an integer of 1 or more, and the maximum value of n is the total number of hydrogens that can be replaced in each compound.

6. The organic electroluminescent compound according to claim 1, wherein the organic electroluminescent compound is used in an N-type charge generation layer.

7. An organic electroluminescent device comprising an organic electroluminescent compound according to claim 1.

8. The organic electroluminescent device according to claim 7, wherein the organic electroluminescent compound is used in an N-type charge generation layer and is doped with an additional metal.

9. The organic electroluminescent device according to claim 8, wherein the metal comprises Yb, Li, Cu, Ag, Au, Al, Mg, or any combination thereof.

10. An organic electroluminescent device comprising a plurality of light-emitting units positioned between a first electrode and a second electrode, and at least one charge generation layer positioned between adjacent light-emitting units among the plurality of light-emitting units, wherein the charge generation layer comprises the organic electroluminescent compound according to claim 1.

11. An organic electroluminescent device according to claim 10, wherein at least one of the plurality of light-emitting units comprises a first light-emitting layer and a second light-emitting layer adjacent to each other.
